(19) United States
(12) Patent Application Publication
(10) Pub. No.: US 2026/0272899 A1
(43) Pub. Date: Sep. 17, 2026

(54) METHODS OF TREATMENT WITH FINERENONE

(71) Applicants: Bayer Aktiengesellschaft, Leverkusen (DE); Mt. Stephen LLC, Brookline, MA (US)

(72) Inventors: Thomas EISSING, Leverkusen (DE); Patrick SCHLOEMER, Leverkusen (DE); Achim FRITSCH, Leverkusen (DE); Thomas WENDL, Leverkusen (DE)

(21) Appl. No.: 19/664,780

(22) Filed: Apr. 30, 2026

Related U.S. Application Data
(63) Continuation-in-part of application No. PCT/EP2025/050103, filed on Jan. 3, 2025.
(60) Provisional application No. 63/617,840, filed on Jan. 5, 2024.
(60) Provisional application No. 63/562,028, filed on Mar. 6, 2024.
(60) Provisional application No. 63/567,145, filed on Mar. 19, 2024.
(60) Provisional application No. 63/568,194, filed on Mar. 21, 2024.
(60) Provisional application No. 63/573,850, filed on Apr. 3, 2024.
(60) Provisional application No. 63/632,257, filed on Apr. 10, 2024.
(60) Provisional application No. 63/635,146, filed on Apr. 17, 2024.
(60) Provisional application No. 63/643,281, filed on May 6, 2024.
(60) Provisional application No. 63/644,084, filed on May 8, 2024.
(60) Provisional application No. 63/651,559, filed on May 24, 2024.
(60) Provisional application No. 63/673,693, filed on Jul. 20, 2024.
(60) Provisional application No. 63/677,471, filed on Jul. 31, 2024.
(60) Provisional application No. 63/683,454, filed on Aug. 15, 2024.
(60) Provisional application No. 63/688,970, filed on Aug. 30, 2024.

(30) Foreign Application Priority Data
Aug. 5, 2024 (EP) 24192813.4

(51) Int. Cl. A61K 31/4375; A61K 9/20; A61K 9/28; A61K 45/06; A61K 47/10; A61P 13/12

(52) U.S. Cl. CPC A61K 31/4375; A61K 9/2013; A61K 9/2054; A61K 9/2095; A61K 9/2893; A61K 45/06; A61K 47/10; A61P 13/12

(57) ABSTRACT

The disclosure refers to a method of preventing or treating heart failure in a patient using (4S)-4-(4-cyano-2-methoxyphenyl)-5-ethoxy-2,8-dimethyl-1,4-dihydro-1,6-naphthyridine-3-carboxamide (INN: Finerenone)

### CROSS-REFERENCE TO RELATED APPLICATIONS

This application is a Continuation in Part of International Patent Application No. PCT/EP2025/064444, filed May 26, 2025, which claims the benefit of and priority to U.S. Provisional Patent Application No. 63/653,291, filed May 30, 2024, the contents of each of which are incorporated herein by reference in their entirety.

### FIELD

The disclosure provides methods for preventing or treating heart failure in a patient using (4S)-4-(4-cyano-2-methoxyphenyl)-5-ethoxy-2,8-dimethyl-1,4-dihydro-1,6-naphthyridine-3-carboxamide (INN: finerenone).

### BACKGROUND

Steroidal Mineralocorticoid Receptor Antagonists (MRAs), spironolactone and eplerenone, are recommended in the treatment of patients with heart failure with reduced ejection fraction (HFrEF) and especially those with ejection fraction below 30%, but their role at higher ejection fractions has not been well established. Broad use of steroidal MRAs has further been limited in part due to safety concerns around risks of hyperkalemia and renal dysfunction. These risks may be reduced by the unique pharmacological properties of the non-steroidal MRA finerenone. Heart failure with preserved ejection fraction (HFpEF) which affects around 50% of the overall heart failure population with increasing prevalence has one of the greatest unmet needs in cardiovascular medicine [Butler et al. Developing therapies for heart failure with preserved ejection fraction: current state and future directions, JACC Heart Fail. 2 (2) (2014) 97-112, https://doi.org/10.1016/j.jchf.2013.10.006]. More recently, the steroidal mineralocorticoid receptor antagonist spironolactone was investigated in patients with HFpEF in a trial called TOPCAT [Pitt et al. Spironolactone for heart failure with preserved ejection fraction, N. Engl. J. Med. 370 (15) (2014) 1383-1392, https://doi.org/10.1056/NEJMoa1313731.]. Although spironolactone did not demonstrate benefit with respect to the primary composite endpoint in this global trial, there was a nominal benefit in patients recruited in North and South America. Subsequent analyses of TOPCAT have revealed significant regional variation of event rates and drug adherence with different treatment effects [Patel et al., Designing future clinical trials in heart failure with preserved ejection fraction: lessons from TOPCAT, Curr. Heart Fail. Rep. 14 (4) (2017) 217-222, https://doi.org/10.1007/s11897-017-0336-x; Pfeffer M A, Claggett B, Assmann S F, Boineau R, Anand I S, Clausell N, Desai A S, Diaz R, Fleg J L, Gordeev I, Heitner J F, Lewis E F, O'Meara E, Rouleau J L, Probstfield J L, Shaburishvili T, Shah S J, Solomon S D, Sweitzer N K, Mckinlay S M, Pitt B. Regional variation in patients and outcomes in the Treatment of Preserved Cardiac Function Heart Failure With an Aldosterone Antagonist (TOPCAT) trial. Circulation. 2015 Jan. 6; 131 (1): 34-42. doi: 10.1161/CIRCULATIONAHA.114.013255. Epub 2014 Nov. 18. PMID: 25406305.].

Finerenone is a novel potent and selective non-steroidal MRA. Finerenone's unique binding mode determines potency, selectivity, and blockade of cofactor association with the MR in the nucleus, while its specific physicochemical properties determine tissue penetration and distribution [Kolkhof et al. Nonsteroidal antagonists of the mineralocorticoid receptor, Curr. Opin. Nephrol. Hypertens. 24 (5) (2015) 417-424, https://doi.org/10.1097/MNH.0000000000000147.]. In addition, pharmacokinetic and drug metabolism properties of finerenone yield a short half-life, no active metabolites, and a renal elimination of less than 1% [R. Heinig et al. Pharmacokinetics of the novel nonsteroidal mineralocorticoid receptor antagonist finerenone (BAY 94-8862) in individuals with renal impairment, Clin. Pharmacol. Drug Dev. 5 (6) (2016) 488-501, https://doi.org/10.1002/cpdd.263., M. Gerisch, et al. Biotransformation of finerenone, a novel nonsteroidal mineralocorticoid receptor antagonist, in dogs, rats, and humans, in vivo and in vitro, Drug Metab. Dispos. 46 (11) (2018) 1546-1555, https://doi.org/10.1124/dmd.118.083337.]. The combination of these major parameters translates into a downstream cardiovascular and renal gene expression profile with a deduced MRA pharmacology that clearly differentiates from steroidal MRAs.

Finerenone's mode of action counteracts multiple components which are also key drivers of the HFpEF pathophysiology [Kolkhof, Lawatscheck, Filippatos and Bakris. Int. J. Mol. Sci. 2022; 23:9243, Sweeney et al. EMBO Mol. Med. 2020; 12: e1086]. Finerenone has been characterized in several preclinical cardiorenal models which develop heart failure and was shown to demonstrate improvement of systolic and diastolic function [Kolkhof et al. Finerenone, a novel selective nonsteroidal mineralocorticoid receptor antagonist protects from rat cardiorenal injury. J. Cardiovasc. Pharmacol. 2014 July; 64 (1): 69-78. https://doi.org/10.1097/FJC.0000000000000091., Grune et al. Selective Mineralocorticoid Receptor Cofactor Modulation as Molecular Basis for Finerenone's Antifibrotic Activity. Hypertension. 2018 April; 71 (4): 599-608. https://doi.org/10.1161/HYPERTENSIONAHA.117.10360., Lachaux et al. Short- and long-term administration of the non-steroidal mineralocorticoid receptor antagonist finerenone opposes metabolic syndrome-related cardio-renal dysfunction. Diabetes Obes. Metab. 2018 October; 20 (10): 2399-2407. https://doi.org/10.1111/dom.13393., Lima Posada et al. Benefits of the non-steroidal mineralocorticoid receptor antagonist finerenone in metabolic syndrome-related heart failure with preserved ejection fraction. Int. J. Mol. Sci. 2023 Jan. 28; 24 (3): 2536.].

However, rodent heart failure models usually develop a mixture of systolic and diastolic dysfunction. There is no rodent animal model which completely recapitulates all aspects of human HFpEF with all aspects [Withaar et al. European Heart Journal (2021) 42, 4420-4430, https://doi.org/10.1093/eurheartj/ehab389]. Therefore, it is currently unknown whether finerenone is able to improve outcomes in human heart failure with preserved ejection fraction (HFpEF), heart failure with mid-range ejection fraction (HFmrEF), heart failure with reduced ejection fraction (HFrEF) or human heart failure (HF) ≥40%. The term heart failure (HF) is sometimes called “cardiac insufficiency” and sometimes HF is still called “cardiac failure.” Sometimes HFrEF is referred to as systolic heart failure (and HFpEF as diastolic HF).

Steroidal mineralocorticoid receptor antagonists reduce morbidity and mortality in patients with heart failure and reduced ejection fraction and in myocardial infarction complicated by left ventricular systolic dysfunction, but their efficacy in those patients with heart failure with mildly reduced or preserved ejection has not been established. According to TOPCAT trial [Already abbreviated above.] (Treatment of Preserved Cardiac Function Heart Failure With an Aldosterone Antagonist), spironolactone did not reduce the primary endpoint in the trial. There remains a high unmet need in patients with heart failure and mildly reduced or preserved ejection fraction despite the recent availability of therapeutic options, including sodium-glucose co-transporter-2 inhibitors.

In contrast to the steroidal MRAs spironolactone and eplerenone, finerenone is a highly selective non-steroidal mineralocorticoid receptor antagonist with distinct physiochemical properties, resulting in a more balanced tissue distribution between the heart and kidney compared with steroidal mineralocorticoid receptor antagonists. In two large outcomes trials in participants with chronic kidney disease and Type-2 diabetes, finerenone was well tolerated and reduced kidney disease progression and cardiovascular events, including heart failure hospitalizations, and is approved for the treatment of these patients in many countries. We designed the Study to Evaluate the Efficacy and Safety of Finerenone on Morbidity and Mortality in Participants With Heart Failure and Left Ventricular Ejection Fraction ≥40% (FINEARTS-HF) trial to test the hypothesis that finerenone, in addition to usual therapy, would reduce the composite of cardiovascular death and total heart failure events in patients with heart failure with mildly reduced or preserved ejection fraction. The FINEARTS-HF trial (see example 2) and its outcomes are described below.

### SUMMARY OF THE INVENTION

An object of the present disclosure is to provide a new method of preventing or treating heart failure.

A further object is to provide a new and/or improved method of treating or preventing symptomatic heart failure.

Another object is to provide a new and/or improved method for reducing the risk of cardiovascular death in patients with heart failure.

A further object is to provide a new and/or improved method of reducing the risk of hospitalization for heart failure in patients.

Another object is to provide a new and/or improved method for reducing the risk of urgent heart failure visits in patients.

A further object is improving symptoms or preventing worsening of symptoms of heart failure. No prior trial of HFmrEF/HFpEF have specifically targeted those with recent worsening heart failure event.

This problem is solved by the method or methods as described below:

The disclosure refers to a method of preventing or treating heart failure in a patient, comprising administering to the patient a therapeutically effective amount of the compound (I)

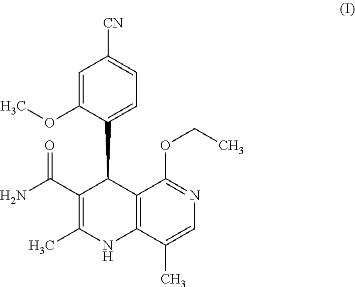 [SMILES: CCOc1ncc(C)c2c1[C@H](c1ccc(C#N)cc1OC)C(C(N)=O)=C(C)N2]

or a hydrate, solvate, pharmaceutically acceptable salt thereof, or a polymorph thereof.

The non-steroidal mineralocorticoid receptor antagonist (MRA) finerenone lessens the effects of mineralocorticoid receptor overactivation by ligands such as aldosterone and cortisol. Finerenone has the chemical name (4S)-4-(4-cyano-2-methoxyphenyl)-5-ethoxy-2,8-dimethyl-1,4-dihydro-1,6-naphthyridine-3-carboxamide, and has the chemical structure of formula (I):

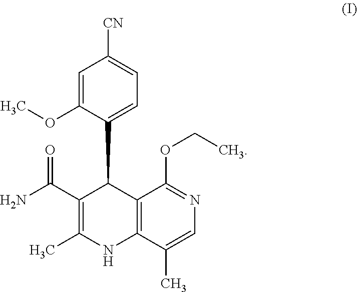 [SMILES: CCOc1ncc(C)c2c1[C@H](c1ccc(C#N)cc1OC)C(C(N)=O)=C(C)N2]

The synthesis, pharmacological properties, and pharmaceutical formulations/dosage forms of finerenone are described in U.S. Pat. No. 8,436,180, which is hereby incorporated by reference herein in its entirety. The use described herein refers to finerenone, a polymorph thereof, a solvate thereof, a hydrate thereof, a salt thereof and/or a pharmaceutically acceptable salt thereof.

### BRIEF DESCRIPTION OF THE DRAWINGS

FIG. 1 provides Schematic overview: Example 2 designed to evaluate the efficacy and safety of finerenone in patients with HF and LVEF ≥40%, with or without diabetes, and across a broad range of renal function.

FIG. 2 provides Example 2 trial, participants with HFmref or HFpEF Cardo-Renal-Metabolic-Overlap.

FIG. 3 provides Eligibility Flowchart.

FIG. 4 provides Cardio-Kidney-Metabolic Overlap FINE-HEART (Example 9).

FIG. 5 provides Recency of Worsening HF Events Before Randomization in Example 2 (FINEARTS-HF): During worsening HF event: n=749 (12%) 7 days or less: n=470 (8%); >7 days to 3 months: n=2028 (34%); >3 months: n=936 (16%); No worsening HF event: n=1,818 (30%). A total of 1,219 patients (20%) had a worsening HF event within 7 days before randomization, with 749 (12%) patients were enrolled during the index event. Of those enrolled during a worsening HF event, 652 (87%) were enrolled during a HF hospitalization and 97 (13%) were enrolled during an outpatient HF visit. 2,028 (34%) patients had a worsening HF event between 7 days and 3 months before randomization, 936 (16%) patients had had a worsening HF event more than 3 months before randomization, and 1,818 (30%) patients had never had a prior worsening HF event. There were notable baseline differences between patients based on the recency of a HF event before randomization. Patients randomized within 7 days of a worsening HF event had higher NYHA functional class, lower KCCQ-TSS, higher baseline NT-proBNP levels, and lower baseline eGFR. The use of SGLT2 inhibitors and ARNIs was also notably higher in those within ≤3 months of a worsening HF event compared with the residual randomized population.

FIG. 6 is a graphical abstract of characteristics of Example 2 (FINEARTS-HF) Participants. Between Sep. 14, 2020 and Jan. 10, 2023, 7,463 patients were screened from 653 sites across 37 countries for enrollment. The primary reason (90%) for screen failure was not meeting ≥1 inclusion or exclusion criterion; 53% had sub-threshold natriuretic peptide levels, 9% had a serum potassium level >5.0 mEq/L, and 28% did not meet other inclusion or exclusion criteria. Overall, 6,016 randomizations to receive finerenone or placebo occurred. Two of these randomizations were identified in connection to the same patient, and both entries were excluded. In addition, thirteen patients from a single site were excluded from the primary analysis due to significant Good Clinical Practice (GCP) violations. In total, 6,001 patients were validly randomized and included in subsequent analyses.

FIG. 7 is a graphical display of the distributions of KDIGO kidney risk among Example 2 participants with available eGFR and UACR data (n=5,797) at baseline. $Graphical display of the distributions of KDIGO kidney risk among weighted NHANES (2015 to March 2020) sample of US adults (weighted n=5,189,186) with HF, age ≥40 years, eGFR ≥25 mL/min/1.73 m², and serum potassium ≥5 mmol/L. § Sankey diagram showing reclassification of KDIGO kidney risk after incorporation of eGFR and UACR, compared with eGFR stages alone. eGFR, estimated glomerular filtration rate; HF, heart failure; KDIGO, Kidney Disease Improving Global Outcomes; NHANES, National Health and Nutrition Examination Surveys; UACR, urine albumin-to-creatinine ratio).

FIG. 8 provides Distribution of KDIGO kidney risk among Example 2 participants by diabetes status: In Example 2, participants without diabetes, 40.7%, 30.4%, 18.4%, and 10.5% were classified as KDIGO low, moderate, high, and very high risk, respectively. In Example 2 participants with diabetes, 26.3%, 27.2%, 22.9%, and 23.6% were classified as KDIGO low, moderate, high, and very high risk, respectively.

FIG. 9 provides a schematic of patient flow of Example 13.

FIG. 10A provides a cumulative incidence plot for the primary endpoint of total cardiovascular deaths and heart failure events. FIG. 10B provides a cumulative incidence plot for total heart failure events.

FIG. 10C provides a Kaplan-Meier plot for death from cardiovascular causes. FIG. 10D provides a Kaplan-Meier plot for first occurrence of cardiovascular death or heart failure event.

FIG. 11 provides a Forest plot of primary treatment effect for prespecified subgroups.

FIGS. 12A-12C provide a Forest plot for prespecified subgroups for time to first cardiovascular death or heart failure event.

FIG. 13 shows a systematic Search of the Literature to Identify Eligible Trials for Inclusion in FINE-HEART, as described in Example 14.

FIG. 14 provides KDIGO Risk Distribution at Baseline in FINE-HEART; Example 14.

FIG. 15 provides Baseline Cardio-Kidney-Metabolic Overlap in FINE-HEART; Example 14.

FIG. 16A provides Key Efficacy Outcomes (CV Death, HF hospitalization, kidney composite endpoint, and all-cause mortality) for each trial. FIG. 16B provides additional efficacy outcomes. FIG. 16C.1 shows the incidence of CV death and composite kidney endpoint. FIG. 16C.2 provides the incidence of HF Hospitalization and All Cause Death. FIG. 16D.1 and FIG. 16D.2 provide a subgroup Forest Plot for the primary endpoint (CV death). see Example 14.

FIGS. 17A-17L provide Kaplan Meier curves illustrating the cumulative incidence of cardiovascular death or hospitalization for heart failure, heart failure hospitalization, cardiovascular death and all-cause death and Nelson-Aalen curves showing the cumulative incidence of total (first and repeat) heart failure hospitalizations and composite of total heart failure hospitalizations and cardiovascular death excluding RALES as time to events was not available; Example 15.

FIGS. 18A-18B provide Event rates in RALES, EMPHASIS-HF, TOPCAT and FINEARTS-HF for each outcome by randomized therapy; Example 15.

FIG. 19A-19B provide Event rates in HFrEF and HFmrEF/HFpEF trials combined and for all trials for each outcome by randomized therapy; Example 15.

FIGS. 20A-20B provide effect of mineralocorticoid receptor antagonist treatment on the pre-specified efficacy outcomes in each of the trials; Example 15.

FIGS. 21A-21C provide effect estimates from the individual patient level meta-analysis of mineralocorticoid receptor antagonists and of cardiovascular death or hospitalization for heart failure, heart failure hospitalization, cardiovascular death, total (first and repeat) heart failure hospitalizations and composite of total heart failure hospitalizations and cardiovascular death, and all-cause death. FINEARTS-HF includes urgent visits for worsening heart failure as a hospitalization equivalent. Estimates from the models in all 4 trials and split by HFrEF and HFmrEF/HFpEF trials with treatment by trial interaction p values are displayed.: Example 15.

FIGS. 22A-22D provide a two stage meta-analysis of cardiovascular death or heart failure hospitalizations, heart failure hospitalizations, cardiovascular death and all cause deaths.

FIGS. 23A-B shows effect of MRAs in each of the trials on pre specified efficacy outcomes including and excluding undetermined deaths from the definition of cardiovascular death; Example 15.

FIG. 24 shows total heart failure hospitalizations and total heart failure hospitalization and cardiovascular deaths analyzed in semi-parametric proportional rates model excluding RALES and including EMPHASIS-HF, TOPCAT and FINEARTS-HF; Example 15.

FIGS. 25A-25B shows sensitivity analysis of the effect of MRAs in each of the trials on pre specified efficacy outcomes using all patients included in RALES, EMPHASIS-HF, FINEARTS-HF and only patients randomized in the Americas in TOPCAT; Example 15.

FIGS. 26A-26D providex the effect of mineralocorticoid receptor antagonist treatment on the composite of cardiovascular death or hospitalization for heart failure (time-to-first event analysis) in key subgroups. FIGS. 26A-26B (Panel A) show the combined RALES and EMPAHASIS-HF trials, and FIG. 26C-26D (Panel B) show the combined TOPCAT and FINEARTS-HF trials. FINEARTS-HF includes urgent visits for worsening heart failure as a hospitalization equivalent; Example 15.

FIG. 27 provides interaction between left ventricular ejection fraction and the effect of randomized treatment on the composite of cardiovascular death or hospitalization for heart failure (left ventricular ejection fraction modelled as a restricted cubic spline). The p-values are p-values for interaction between left ventricular ejection fraction and effect of mineralocorticoid receptor antagonist treatment in the trials including patients with heart failure and reduced ejection fraction (HFrEF) and in the trials including patients with heart failure and mildly reduced and preserved ejection fraction (HFmrEF/HFpEF). FINEARTS-HF includes urgent visits for worsening heart failure as a hospitalization equivalent; Example 15.

FIGS. 28A-28B provide subgroup analysis of the outcome of cardiovascular death or first hospitalization for heart failure in all four trials; Example 15.

FIGS. 29A-29B provide the effect of mineralocorticoid receptor antagonist treatment on additional pre-specified safety outcomes in each trial; Example 15.

FIGS. 30A-30C provide Table 9 Safety outcomes in HFrEF and HFmrEF/HFpEF trials combined and all 4 trials combined; Example 15.

FIGS. 31A-31B provide effect of mineralocorticoid receptor antagonist treatment on the pre-specified safety outcomes in each trial eGFR<45; Example 15.

FIGS. 32A-32B provide effect of mineralocorticoid receptor antagonist treatment on the pre-specified safety outcomes in each trial eGFR≥45; Example 15.

FIGS. 33A-33B provide effect of mineralocorticoid receptor antagonist treatment on the pre-specified safety outcomes in each trial TOPCAT Americas; Example 15.

FIG. 34 provides effects of finerenone compared with placebo on outcomes according to BMI category; Example 16.

FIG. 35 provides effect of finerenone compared with placebo on cardiovascular death and total worsening HF events according to continuous BMI; Example 16.

FIGS. 36A-36B provide key trial outcomes according to sex and treatment in FINEARTS-HF, Example 17. FIG. 36A provides the primary endpoint, and FIG. 36B provides total HF events.

FIG. 37 shows the effect of finerenone according to sex in FINEARTS-HF, Example 17.

FIG. 38 shows the primary outcome after 1 month (landmark analysis) according to initial decline (“dip”) in eGFR ≥15% or not and randomized treatment assignment (finerenone or placebo), Example 18.

FIG. 39 shows the effect of finerenone on the prespecified hierarchical composite endpoint, Example 19.

FIGS. 40A-40C provide win statistics over time, Example 19. FIG. 40A provides win ratio, and FIG. 40B provides win odds. FIG. 40C provides the percentage of wins or losses.

FIG. 41 provides Baseline potassium levels, Example 20.

FIG. 42 provides Spectrum of potassium related changes, Example 20.

FIG. 43 provides Risk of total worsening HF Events and CV death across a range of potassium levels after 1 month of treatment, Example 20.

FIGS. 44A-44B provides Treatment Effects of Finerenone on Systolic Blood Pressure in the FINEARTS-HF Trial. FIG. 44A provides profiles of Systolic Blood Pressure in FINEARTS-HF, by Treatment Arm. FIG. 44B provides treatment effects of Finerenone on Systolic Blood Pressure Between Baseline and Month 1, by Baseline BP Category. FIGS. 44A-44B display mean values of systolic blood pressure by treatment arm at baseline and each post-baseline visit (FIG. 44A) and treatment effects of finerenone on systolic blood pressure at 1 month, according to blood pressure category at baseline (FIG. 44B). Abbreviations: aTRH=apparent treatment-resistant hypertension; BP=blood pressure; HTN=hypertension; SBP=systolic BP.

FIGS. 45A-45B provide the probability of death following a worsening heart failure (HF) event (FIG. 45A) and the effect of finerenone on outpatient oral diuretic intensification (FIG. 45B). FIG. 45A shows the cumulative incidence of all-cause death following each type of worsening heart failure (HF) event, or from randomization for patients who did not experience worsening HF. FIG. 45B shows the cumulative incidence of outpatient oral diuretic intensification in patients randomized to finerenone compared to placebo. Hazard ratio (HR) compares finerenone to placebo in a Cox regression model stratified by region and ejection fraction dichotomized at 60%. See Example 22.

FIGS. 46A-46B provide baseline characteristics (FIG. 46A) and treatment response of participants with history of LVEF <40% and those with LVEF consistently ≥40% (FIG. 46B), Example 23.

FIG. 47 provides shows finerenone, bilirubin, and heart failure with mildy reduced or preserved ejection fraction (an analysis from FINEARTS-HF), Continuous bilirubin, Models were adjusted for geographic region and baseline LVEF, Example 24.

FIG. 48 provides Distribution of KCCQ-TSS. Kansas City Cardiomyopathy Questionnaire total symptom score. Example 25.

FIGS. 49A-49D provide mean cumulative incidence of clinical outcomes according to baseline KCCQ-TSS category divided by tertile. (FIG. 49A) CV death and total HF events; (FIG. 49B) HF events; (FIG. 49C) CV death; and (FIG. 49D) all-cause death. Example 25.

FIGS. 50A-50D provide incidence rates of outcomes across baseline KCCQ-TSS. (FIG. 50A) CV death and total HF events; (FIG. 50B) HF events; (FIG. 50C) CV death; (FIG. 50D) all-cause death. HF, heart failure. Placebo: red (upper line); finerenone: blue (lower line). KCCQ-TSS, Kansas City Cardiomyopathy Questionnaire Total Symptom Score. Example 25.

FIGS. 51A-51C provide adjusted mean difference in KCCQ-CSS, -TSS, -OSS, and subdomains by randomized treatment allocation at 6 (FIG. 51A), 9 (FIG. 51B), and 12 months (FIG. 51C). KCCQ, Kansas City Cardiomyopathy Questionnaire; CSS, Clinical Summary Score; OSS, Overall Summary Score; TSS, Total Symptom Score. See Example 25.

FIG. 52 provides Overall average mean difference in KCCQ-CSS, TSS, OSS, and subdomains attributable to finerenone over the follow-up period (across 0, 6, 9, and 12 months). KCCQ, Kansas City Cardiomyopathy Questionnaire; CSS, Clinical Summary Score; OSS, Overall Summary Score; TSS, Total Symptom Score. See Example 52.

FIG. 53 provides treatment Effects of Finerenone on Primary Endpoint by Concomitant SGLT2 Inhibitor Use. Abbreviations: ARR=absolute risk reduction; CI=confidence interval; CV=cardiovascular; HF=heart failure; py=patient-years; SGLT2i=sodium-glucose-co-transporter-2 inhibitor; RR=rate ratio. See Example 27.

FIGS. 54A-54B provide New Initiations (FIG. 54B) and Discontinuations (FIG. 54A) of SGLT2i During Trial Follow-up. Abbreviations: CI=confidence interval; HR=hazard ratio; SGLT2i=sodium-glucose-co-transporter-2 inhibitor. See Example 27.

FIG. 55 provides CYP3A4 inhibitor updates.

FIG. 56 provides Subgroup analysis of FINEARTS-HF and FIDELITY.

FIG. 57 show hyperkalemia and use of CYP3A4 inhibitors at baseline in clinical Phase 3 trials FINEARTS-HF and FIDELITY.

FIGS. 58A-58B show hperkalemia by extent of co-medication use of CYP3A4 inhibitors.

FIG. 59 provides Potassium >5.5 mmol/l and use of CYP3A4 inhibitors at baseline in in clinical Phase 3 trials FINEARTS-HF and FIDELITY.

FIGS. 60A-60B provide Model-Informed Drug Development (MIDD) “subgroup” analysis-Serum potassium levels. [R. Madabushi et al.; Pharmaceutical Research (2022) 39:1669-1680. doi: 10.1007/s11095-022-03288-w.]

FIG. 61 provides MIDD cardiac safety assessment-Concentration-QTc analysis of TQT study data (upper panel) and estimated FINEARTS-HF maximal concentrations.

FIG. 62A provides finerenone plasma concentration-time profiles of PBPK population simulations of 20 mg finerenone concomitant with 200 mg BID itraconazole (red) and 40 mg finerenone concomitant with 500 mg TID erythromycin (blue); time corresponds to time after the finerenone dose. FIG. 62B provides Finerenone plasma concentration-time profiles of PBPK population simulations of 10 mg finerenone concomitant with 200 mg BID itraconazole (red) and 20 mg finerenone concomitant with 500 mg TID erythromycin (blue); time corresponds to time after the finerenone dose. FIG. 62C provides Finerenone plasma concentration-time profiles of PBPK population simulations of 10 mg finerenone concomitant with 200 mg BID itraconazole (red) and 40 mg finerenone concomitant with 500 mg TID erythromycin (blue); time corresponds to time after the finerenone dose.

FIG. 63A is a forest plot of PBPK population simulated AUC values of finerenone in doses of 10, 20 and 40 mg under concomitant administration of strong (itraconazole and clarithromycin), moderate (erythromycin and verapamil), moderate to weak (fluvoxamine, classified as weak CYP3A4 inhibitor until 2019, afterwards as moderate) and weak (cimetidine) CYP3A4 inhibitors, dots represent the median and the whiskers represent the 5th to 95th percentiles. FIG. 63B is a forest plot of PBPK population simulated Cmax values of finerenone in doses of 10, 20 and 40 mg under concomitant administration of strong (itraconazole and clarithromycin), moderate (erythromycin and verapamil), moderate to weak (fluvoxamine, classified as weak CYP3A4 inhibitor until 2019, afterwards as moderate) and weak (cimetidine) CYP3A4 inhibitors, dots represent the median and the whiskers represent the 5th to 95th percentiles.

FIG. 64 shows plasma concentration-time profiles of population simulations of 10 mg finerenone concomitant with 200 mg BID itraconazole (red) and 40 mg finerenone concomitant with 500 mg TID erythromycin (blue), time corresponds to time after the finerenone dose.

FIG. 65 shows plasma concentration-time profiles of population simulations of 20 mg finerenone concomitant with 200 mg BID itraconazole (red) and 40 mg finerenone concomitant with 500 mg TID erythromycin (blue), time corresponds to time after the finerenone dose.

FIG. 66 shows plasma concentration-time profiles of population simulations of 10 mg finerenone concomitant with 200 mg BID itraconazole (red) and 20 mg finerenone concomitant with 500 mg TID erythromycin (blue), time corresponds to time after the finerenone dose.

FIG. 67 shows plasma concentration-time profiles of population simulations of 10 mg finerenone concomitant with 200 mg BID itraconazole (red) and 40 mg finerenone alone (control).

FIG. 68 is a forest plot of simulated AUC values of finerenone in doses of 2.5, 5, 10, 20 and 40 mg under concomitant administration of strong (itraconazole and clarithromycin), moderate (erythromycin and verapamil), moderate to weak (fluvoxamine, classified as weak CYP3A4 inhibitor until 2019, afterwards as moderate) and weak (cimetidine) CYP3A4 inhibitors, dots represent the median and the whiskers represent the 5th to 95th percentiles.

FIG. 69 is a forest plot of simulated Cmax values of finerenone in doses of 2.5, 5, 10, 20 and 40 mg under concomitant administration of strong (itraconazole and clarithromycin), moderate (erythromycin and verapamil), moderate to weak (fluvoxamine, classified as weak CYP3A4 inhibitor until 2019, afterwards as moderate) and weak (cimetidine) CYP3A4 inhibitors, dots represent the median and the whiskers represent the 5th to 95th percentiles.

FIG. 70 shows time profiles of finerenone for finerenone 5 mg and verapamil compared to simulated finerenone for finerenone 5 mg control. Simulation time range 78-102 h for finerenone-verapamil interaction. Time profiles are plotted in logarithmic scale. For all simulations: n=1000. For all observed data: n=13.

### DETAILED DESCRIPTION

### Definitions

For interpreting this specification, the following definitions will apply. If any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

Whenever appropriate, terms used in the singular will include the plural and vice versa. The use of “a” herein means “one or more” unless stated otherwise or where the use of “one or more” is clearly inappropriate. The use of “or” means “and/or” unless stated otherwise. The use of “comprise”, “comprises” “comprising”, “include”, “Includes”, and “including” are interchangeable and are not limiting. The terms “such as,” “for example,” and “e.g.” are not intended to be limiting. For example, the term “including” shall mean “including, but not limited to”.

The “dose of finerenone recommended for an otherwise similar patient who is not co-administered the strong CYP3A4 inhibitor” refers to the dosage of finerenone recommended for the patient based on the patient's indication and characteristics as indicated on the Prescribing Information for KERENDIA (finerenone) as revised July 2025 (referred to as the “FDA label,” “FDA-approved label” or the “KERENDIA label”). As shown on this label, finerenone is approved for different indications. The indication and certain relevant characteristics of the patient, such as estimated glomerular filtration rate (eGFR) and current serum potassium levels, influence the recommended dosage of finerenone. The July 2025 label contraindicates coadministration of finerenone with strong CYP3A4 inhibitors. The FDA-approved label also recommends starting doses for initiating treatment (referred to as “starting daily doses”) and maintenance doses (referred to as “target daily doses”) for effective treatment. As shown in, e.g., Section 2.3, of the KERENDIA label, the target daily dose may be adjusted based on the patient's current serum potassium and/or eGFR, and this dose is referred to as the “current dose” or “current KERENEDIA dose” on the FDA-approved label. As used herein, the “target daily doses” encompasses the “current doses” recommended for a patient based on the patient's current serum potassium, which are found in Tables 2 and 3 of Section 2.3 of the July 2025 label. Thus, “recommended dose,” as used herein, is selected based on the patient's condition and any relevant characteristics (e.g., eGFR and/or current serum potassium levels), and the phase of treatment (starting dose, current dose or target dose) as described on the FDA-approved label.

All references to “finerenone” include its pharmaceutically acceptable salts, solvates, hydrates, and polymorphs thereof (e.g., the known polymorphs described herein), unless otherwise stated.

As used herein, “dose” refers to the total amount of finerenone administered in a dosing event (i.e., to achieve the once daily doses described in the KERENDIA label or described herein). To provide the dose, one or more oral dosage forms (e.g., tablets) may be administered. In embodiments, one, two, three, or four oral dosage forms (e.g., tablets) are administered to provide the scheduled dose. In embodiments, one or two oral dosage forms (e.g., tablets) are administered to provide the scheduled dose. The oral dosage form can be whole tablet or it can be a divided tablet, e.g., half a tablet or a quarter of a tablet. In some embodiments, the oral dosage form (e.g., tablet) may be crushed and mixed with water or soft food (e.g., apple sauce). The oral dosage form may be an oral granule for suspension in a liquid (e.g., water) or mixed with a soft food (e.g., apple sauce).

“Solvates” for the purposes of the disclosure are those forms of the compounds or their salts where solvent molecules form a stoichiometric complex in the solid state and include, but are not limited to for example water, ethanol and methanol.

“Hydrates” are a specific form of solvates, where the solvent molecule is water. Hydrates of the compounds of the disclosure or their salts are stoichiometric compositions of the compounds or salts with water, such as, for example, monohydrate, dihydrates, trihydrate, hemihydrate, sesquihydrate.

“Salts” for the purposes of the present disclosure are preferably “pharmaceutically acceptable salts” of finerenone. Suitable pharmaceutically acceptable salts that can be used in the combination according to the disclosure are well known to those skilled in the art and include salts of inorganic acids, organic acids, inorganic bases, alkaline cations, alkaline earth cations and organic bases. In one embodiment the pharmaceutically acceptable salt can be selected from mandelic acid acetate, benzoate, besylate, bromide, camsylate, carbonate, citrate, edisylate, estolate, fumarate, gluceptate, gluconate, glucuronate, hippurate, iodide, isethionate, lactate, lactobionate, malate, maleate, mesylate, methylsulfate, napsylate, nitrate, oxalate, pamoate, phosphate, stearate, succinate, sulfate, tartrate, bitartrate, tosylate, calcium, diolamine, lithium, lysine, magnesium, meglumine, N-methylglucamine, olamine, potassium, tromethamine, tris (hydroxymethyl)aminomethane, benzenesulfonate, ethanesulfonate and zinc. Salts and/complexes can also be formed with at least one of the following acids: hydrochloric acid, hydrobromic acid, sulfuric acid, phosphoric acid, methane sulphonic acid, trifluoromethanesulfonic acid, benzenesulfonic acid, p-toluene sulfonic acid, 1-naphthalenesulfonic acid, 2-naphthalenesulfonic acid, acetic acid, trifluoroacetic acid, malic acid, tartaric acid, citric acid, lactic acid, oxalic acid, succinic acid, fumaric acid, maleic acid, benzoic acid, salicylic acid, and/or phenylacetic acid. Furthermore, the compound according to formula (I) can also form co-crystals with the aforementioned acids.

“Pharmaceutically acceptable” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The phrase “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound disclosed herein with acids. Pharmaceutically acceptable salts are also obtained by reacting a compound disclosed herein with a base to form a salt.

In one embodiment the pharmaceutically acceptable salt can be selected from hydrochloride, sulfate, mesylate, tosylate, tartrate, citrate, benzenesulfonate, ethanesulfonate, maleate, and phosphate.

Polymorphic form of finerenone is disclosed in U.S. Pat. No. 10,399,977, which is hereby incorporated herein by reference in its entirety. In the methods of the present disclosure, the compound to be administered may be finerenone of the formula (I) in crystalline form of polymorph I characterized in that the x-ray diffractogram of the compound exhibits peak maxima of the 2-theta angle at 8.5, 14.1, 17.2, 19.0, 20.5, 25.6, 26.5 degrees. In the methods of the present disclosure, the compound to be administered may be finerenone of the formula (I) in crystalline form of polymorph I characterized in that the IR spectrum (IR-ATR) of the compound exhibits band maxima at 3475, 2230, 1681, 1658, 1606, 1572, 1485, 1255, 1136 and 1031 cm−1. In the methods of the present disclosure, the compound to be administered may be finerenone of the formula (I) in crystalline form of polymorph I characterized in that the Raman spectrum of the compound exhibits band maxima at 3074, 2920, 2231, 1601, 1577, 1443, 1327, 1267, 827 and 155 cm−1. Experimental conditions for the measurement of these crystalline form parameters are found in the examples.

In one embodiment, finerenone of the formula (I) in crystalline form of polymorph I is used. In one embodiment, finerenone is the compound of the formula (I) in crystalline form of polymorph I

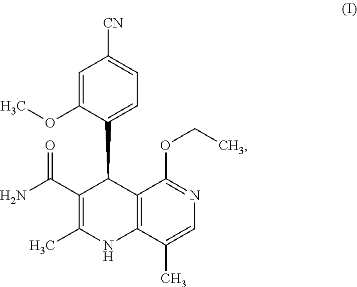 [SMILES: CCOc1ncc(C)c2c1[C@H](c1ccc(C#N)cc1OC)C(C(N)=O)=C(C)N2]

wherein the x-ray diffractogram of the compound exhibits peak maxima of the 2-theta angle at 8.5, 14.1, and 19.0 degrees. In one embodiment, finerenone is the compound of the formula (I) in crystalline form of polymorph I, wherein the x-ray diffractogram of the compound further exhibits any one f-peak maxima of the 2-theta angle at 17.2, 20.5, 25.6, and 26.5. In one embodiment, finerenone is the compound of the formula (I) in crystalline form of polymorph I, wherein the IR spectrum of the compound exhibits any one of band maxima at 3475, 2230, 1681, 1658, 1606, 1572, 1485, 1255, 1136 and 1031 cm−1. In one embodiment, finerenone is the compound of the formula (I) in crystalline form of polymorph I, wherein the IR spectrum of the compound exhibits band maxima at 3475, 2230, 1681, 1658, 1606, 1572, 1485, 1255, 1136 and 1031 cm−1. In one embodiment, finerenone is the compound of the formula (I) in crystalline form of polymorph I, wherein the Raman spectrum of the compound exhibits any one of band maxima at 3074, 2920, 2231, 1601, 1577, 1443, 1327, 1267, 827 and 155 cm−1. In one embodiment, finerenone is the compound of the formula (I) in crystalline form of polymorph I, wherein the Raman spectrum of the compound exhibits band maxima at 3074, 2920, 2231, 1601, 1577, 1443, 1327, 1267, 827 and 155 cm−1. In one embodiment, finerenone is the compound of the formula (I) in crystalline form of polymorph I, wherein the compound has a melting point of 252° C.

In one embodiment the method according to the disclosure is used for the treatment and/or prevention of heart failure.

In one embodiment, the heart failure is selected from the group consisting of symptomatic heart failure, heart failure with improved ejection fraction (HFimpEF), heart failure with mid-range ejection fraction (HFmrEF), heart failure preserved ejection fraction (HFpEF), heart failure with reduced ejection fraction (HFrEF), chronic heart failure (CHF), congestive heart failure, acute heart failure, chronic heart failure, worsening chronic heart failure (WCHF), and hospitalization for heart failure.

In one embodiment, the heart failure is symptomatic heart failure. In one embodiment, the heart failure is heart failure with improved ejection fraction (HFimpEF). In one embodiment, the heart failure is heart failure with mid-range ejection fraction (HFmrEF). In one embodiment, the heart failure is preserved ejection fraction (HFpEF). In one embodiment, the heart failure is heart failure with reduced ejection fraction (HFrEF). In one embodiment, the heart failure is chronic heart failure (CHF). In one embodiment, the heart failure is congestive heart failure. In one embodiment, the heart failure is acute heart failure. In one embodiment, the heart failure is chronic heart failure. In one embodiment, the heart failure is worsening chronic heart failure (WCHF). In one embodiment, the heart failure is hospitalization for heart failure.

In one embodiment, the method according to the disclosure improves symptoms of heart failure. In this embodiment the heart failure can be selected from the group consisting of symptomatic heart failure, heart failure with improved ejection fraction (HFimpEF), heart failure with mid-range ejection fraction (HFmrEF), heart failure preserved ejection fraction (HFpEF), heart failure with reduced ejection fraction (HFrEF), chronic heart failure (CHF), congestive heart failure, acute heart failure, chronic heart failure, worsening chronic heart failure (WCHF), and hospitalization for heart failure.

In one embodiment, the method according to the disclosure prevents worsening of symptoms of heart failure. In this embodiment the heart failure can be selected from the group consisting of symptomatic heart failure, heart failure with improved ejection fraction (HFimpEF), heart failure with mid-range ejection fraction (HFmrEF), heart failure preserved ejection fraction (HFpEF), heart failure with reduced ejection fraction (HFrEF), chronic heart failure (CHF), congestive heart failure, acute heart failure, chronic heart failure, worsening chronic heart failure (WCHF), and hospitalization for heart failure.

In one embodiment, the hospitalization duration for heart failure is equal to or below 6 months. In one embodiment, the hospitalization duration for heart failure was equal to or above 6 months. In one embodiment, the hospitalization duration for heart failure is equal to or below 12 months. In one embodiment, the hospitalization duration for heart failure is equal to or greater than 12 months. In one embodiment, the hospitalization duration for heart failure is any prior heart failure hospitalization of any duration.

In one embodiment, the hospitalization duration for heart failure is equal to or below 6 months prior to randomization. In one embodiment, the hospitalization duration for heart failure was equal to or above 6 months prior to randomization. In one embodiment, the hospitalization duration for heart failure is equal to or below 12 months prior to randomization. In one embodiment, the hospitalization duration for heart failure is equal to or above 12 months prior to randomization. In one embodiment, the hospitalization duration for heart failure is any prior heart failure hospitalization of any duration prior to randomization.

In one embodiment, the hospitalization duration for heart failure is equal to or below 6 months prior to treatment with finerenone. In one embodiment, the hospitalization duration for heart failure was equal to or above 6 months prior to treatment with finerenone. In one embodiment, the hospitalization duration for heart failure is equal to or below 12 months prior to treatment with finerenone. In one embodiment, the hospitalization duration for heart failure is equal to or above 12 months prior to treatment with finerenone. In one embodiment, the hospitalization for heart failure is any prior heart failure hospitalization of any duration prior to treatment with finerenone.

Unless otherwise stated, the terms used herein are used in the manner customary to those skilled in the art. The chemical definitions are used according to IUPAC.

The term “equal to or below” or “S” is used in the sense of two alternatives. These alternatives are, on the one hand, that a number and/or range is either less than a certain number and/or a specific range, or on the other hand, that a number and/or range is both equal to and less than a certain number and/or a specific range, optionally only less than or either equal to or less than a certain number and/or a specific range.

The term “equal to or above” or “≥” is used in the sense of two alternatives. These alternatives are, on the one hand, that a number and/or range is either greater than a certain number and/or a specific range, or on the other hand, that a number and/or range is both equal to and greater than a certain number and/or a specific range, optionally only greater than or either equal to or greater than a certain number and/or a specific range. In each instance, the two alternatives are utilized as interchangeable options, defined as two or more options that can be substituted for one another in the context of synonyms

In one embodiment, the “heart failure” is classified by the definition of the New York Heart Association (NYHA) Functional Classification (The Criteria Committee of the New York Heart Association. (1994). Nomenclature and Criteria for Diagnosis of Diseases of the Heart and Great Vessels (9th ed.). Boston: Little, Brown & Co. pp. 253-256.). This definition is considered a way of describing the extent of functional limitation due to symptoms caused by HF. It places patients in one of four categories based on how much they are limited during physical activity; the limitations/symptoms are in regard to normal breathing and varying degrees in shortness of breath and/or angina. In practice, sometimes only two symptoms are considered relevant.

NYHA Class I (Symptoms): Presence of cardiac disease. No limitation of physical activity. Ordinary physical activity does not cause undue fatigue, palpitation, dyspnea (shortness of breath). NYHA Class II (Symptoms): Slight limitation of physical activity. Comfortable at rest. Ordinary physical activity results in fatigue, palpitation, dyspnea. NYHA Class III (Symptoms): Marked limitation of physical activity. Comfortable at rest. Less than ordinary activity causes fatigue, palpitation, or dyspnea. NYHA Class IV (Symptoms): Unable to carry on any physical activity without discomfort. Symptoms of heart failure at rest. If any physical activity is undertaken, discomfort increases.

In one embodiment, the heart failure is selected from New York Heart Association (NYHA) class I, II, III and IV. In one embodiment, the heart failure is selected from NYHA class II, III and IV. In one embodiment, the heart failure is NYHA class II. In one embodiment, the heart failure is NYHA class III. In one embodiment, the heart failure is NYHA class IV.

In one embodiment, method for treating symptomatic heart failure in a patient, comprising administering to the patient a therapeutically effective amount of the compound (I)

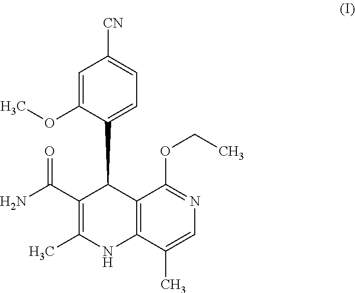 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for preventing symptomatic heart failure in a patient, comprising administering to the patient a therapeutically effective amount of the compound (I)

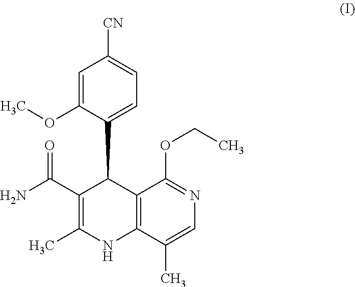 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for lowering the risk of symptomatic heart failure in a patient, comprising administering to the patient a therapeutically effective amount of the compound (I)

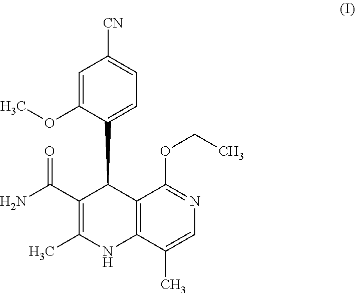 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for treating symptomatic heart failure with left ventricular ejection fraction (LVEF) equal to or above 30% in a patient, comprising administering to the patient a therapeutically effective amount of the compound (I)

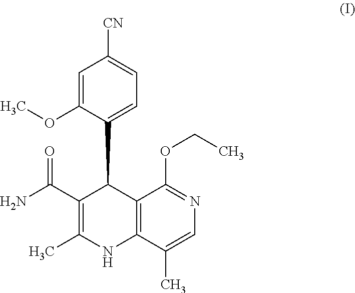 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for preventing symptomatic heart failure with left ventricular ejection fraction (LVEF) equal to or above 30% in a patient, comprising administering to the patient a therapeutically effective amount of the compound (I)

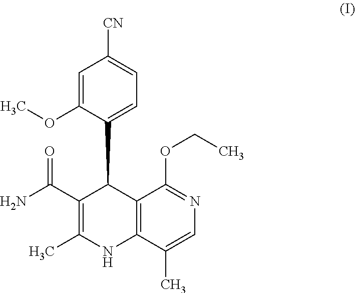 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for lowering the risk of symptomatic heart failure with left ventricular ejection fraction (LVEF) equal to or above 30% in a patient, comprising administering to the patient a therapeutically effective amount of the compound (I)

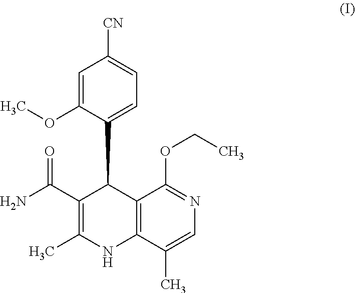 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for treating symptomatic heart failure with left ventricular ejection fraction (LVEF) of equal to or above 40% in a patient, comprising administering to the patient a therapeutically effective amount of the compound (I)

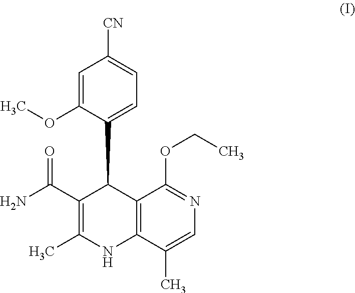 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for preventing symptomatic heart failure with left ventricular ejection fraction (LVEF) equal to or above 40% in a patient, comprising administering to the patient a therapeutically effective amount of the compound (I)

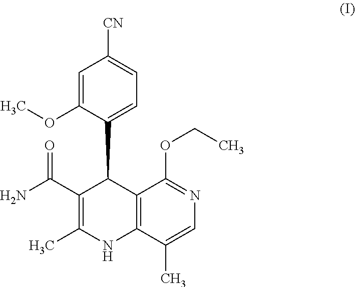 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for lowering the risk of symptomatic heart failure with left ventricular ejection fraction (LVEF) equal to or above 40% in a patient, comprising administering to the patient a therapeutically effective amount of the compound (I)

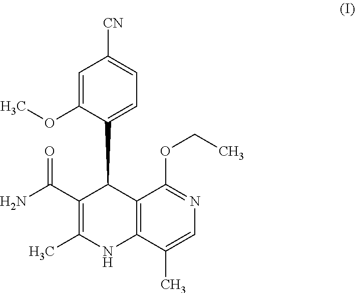 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for reducing the risk of cardiovascular death, hospitalization for heart failure, and/or urgent heart failure visits in patients with heart failure, comprising administering to the patient a therapeutically effective amount of the compound (I)

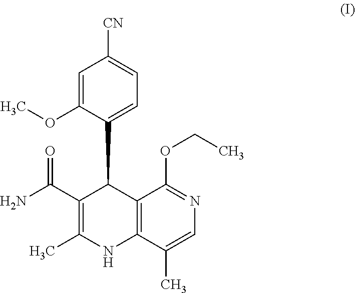 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for reducing the risk of cardiovascular death, hospitalization for heart failure, and/or urgent heart failure visits in patients with heart failure with left ventricular ejection fraction (LVEF) equal to or above 30%, comprising administering to the patient a therapeutically effective amount of the compound (I)

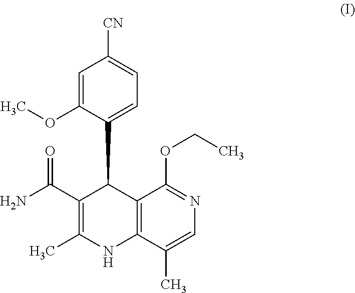 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for reducing the risk of cardiovascular death, hospitalization for heart failure, and/or urgent heart failure visits in patients with heart failure with left ventricular ejection fraction (LVEF) equal to or above 40%, comprising administering to the patient a therapeutically effective amount of the compound (I)

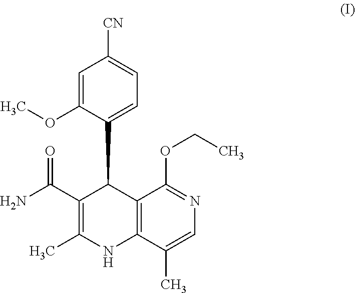 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for reducing the risk of cardiovascular death in patients with heart failure, comprising administering to the patient a therapeutically effective amount of the compound (I)

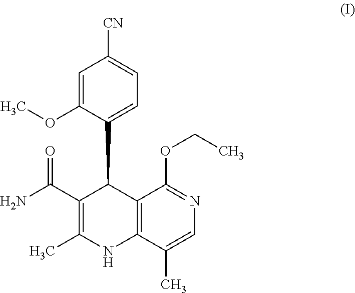 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for reducing the risk of cardiovascular death in patients with heart failure with left ventricular ejection fraction (LVEF) equal to or above 30%, comprising administering to the patient a therapeutically effective amount of the compound (I)

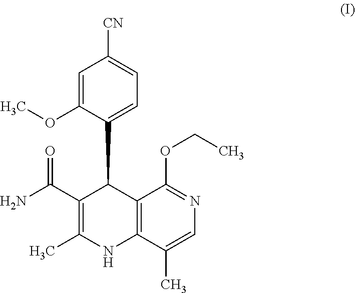 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for reducing the risk of cardiovascular death in patients with heart failure with left ventricular ejection fraction (LVEF) equal to or above 40%, comprising administering to the patient a therapeutically effective amount of the compound (I)

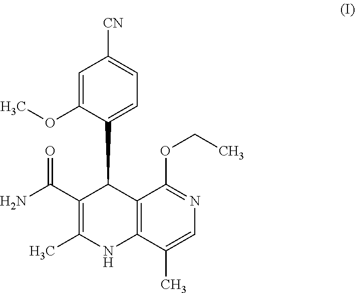 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for reducing the risk of hospitalization for heart failure in patients with heart failure, comprising administering to the patient a therapeutically effective amount of the compound (I)

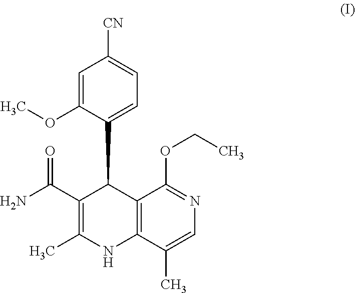 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for reducing the risk of hospitalization for heart failure in patients with heart failure with left ventricular ejection fraction (LVEF) equal to or above 30%, comprising administering to the patient a therapeutically effective amount of the compound (I)

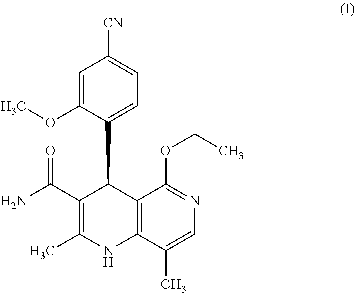 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for reducing the risk of hospitalization for heart failure in patients with heart failure with left ventricular ejection fraction (LVEF) equal to or above 40%, comprising administering to the patient a therapeutically effective amount of the compound (I)

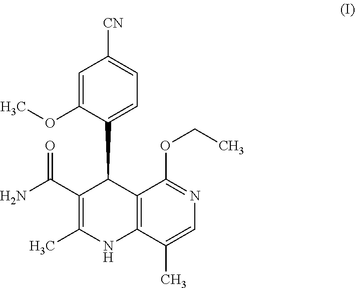 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for reducing the risk of urgent heart failure visits in patients with heart failure, comprising administering to the patient a therapeutically effective amount of the compound (I)

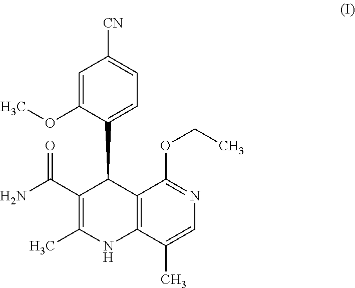 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for reducing the risk of urgent heart failure visits in patients with heart failure with left ventricular ejection fraction (LVEF) of equal to or above 30%, comprising administering to the patient a therapeutically effective amount of the compound (I)

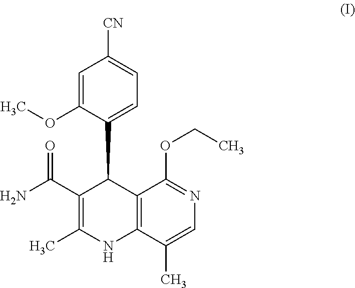 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method for reducing the risk of urgent heart failure visits in patients with heart failure with left ventricular ejection fraction (LVEF) of equal to or above 40%, comprising administering to the patient a therapeutically effective amount of the compound (I)

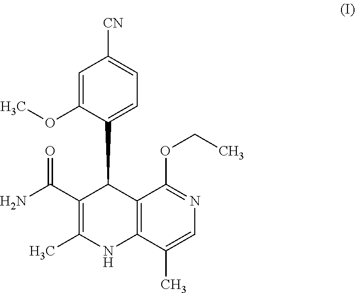 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

In one embodiment, the present disclosure provides a method that improves symptoms of heart failure with left ventricular ejection fraction (LVEF) equal to or above 40%, comprising administering to the patient a therapeutically effective amount of the compound (I)

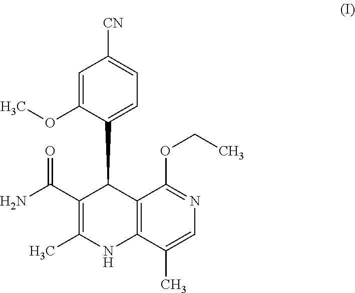 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof. The heart failure is selected from the group consisting of symptomatic heart failure, heart failure with improved ejection fraction (HFimpEF), heart failure with mid-range ejection fraction (HFmrEF), heart failure preserved ejection fraction (HFpEF), heart failure with reduced ejection fraction (HFrEF), chronic heart failure (CHF), congestive heart failure, acute heart failure, chronic heart failure, worsening chronic heart failure (WCHF), and hospitalization for heart failure.

In one embodiment, the present disclosure provides a method that prevents worsening of symptoms of heart failure with left ventricular ejection fraction (LVEF) equal to or above 40%, comprising administering to the patient a therapeutically effective amount of the compound (I)

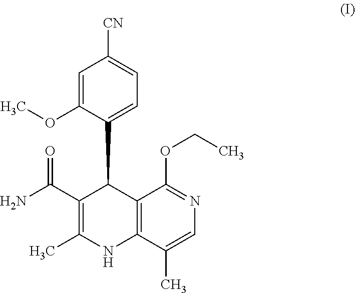 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof. The heart failure is selected from the group consisting of symptomatic heart failure, heart failure with improved ejection fraction (HFimpEF), heart failure with mid-range ejection fraction (HFmrEF), heart failure preserved ejection fraction (HFpEF), heart failure with reduced ejection fraction (HFrEF), chronic heart failure (CHF), congestive heart failure, acute heart failure, chronic heart failure, worsening chronic heart failure (WCHF), and hospitalization for heart failure.

In one embodiment, the present disclosure provides a method for reducing the risk of cardiovascular death, hospitalization for heart failure, and/or urgent heart failure visits in adults with heart failure with left ventricular ejection fraction (LVEF) of ≥40%

In one embodiment, the patient has heart failure with left ventricular ejection fraction (LVEF) of equal to or below 30%.

Left ventricular ejection fraction (LVEF) is the central measure of left ventricular function. This indication of how well one's heart is pumping out blood can help to diagnose and track heart failure. LVEF is the fraction of chamber volume ejected in systole (stroke volume) in relation to the volume of the blood in the ventricle at the end of diastole (end-diastolic volume). It is a measurement, expressed as a percentage (%), of how much blood the left ventricle pumps out with each contraction. An LVEF of 60% means that 60% of the total amount of blood in the left ventricle is pushed out with each heartbeat. A normal heart's LVEF is between 55 and 70%. The simplest classification as per the American College of Cardiology (ACC) that is used clinically as follows:
Hyperdynamic=LVEF greater than 70%
Normal=LVEF 50% to 70% (midpoint 60%)
Mild dysfunction=LVEF 40% to 49% (midpoint 45%)
Moderate dysfunction=LVEF 30% to 39% (midpoint 35%)
Severe dysfunction=LVEF less than 30%

LVEF may be measured using a non-invasive method or an invasive method. Non-invasive methods for measuring LVEF include echocardiography, MRI, CT, gated equilibrium radionuclide angiography (MUGA scan), and gated myocardial perfusion imaging with either single-photon emission computed tomography (SPECT) or positron emission tomography (PET). There are also invasive methods. Ejection fraction is then calculated from volume estimates based on this imaging. The following publications describe how to measure LVEF and are incorporated by reference herein in their entirety: Foley et al., Measuring Left Ventricular Ejection Fraction-Techniques and Potential Pitfalls, European Cardiology 2012; 8 (2): 108-14; Lang et al. Guidelines and Standards; Recommendations for Cardiac Chamber Quantification by Echocardiography in Adults: An Update from the American Section of Echocardiography and the European Association of Cardiovascular Imaging 2015; 28 (1): 1-39.

In one embodiment, the patient has a LVEF selected from the group consisting of less than 30%, 30% to 39%, 40% to 49%, 45%, 50% to 70%, 60%, and greater than 70%. In one embodiment, the patient has a LVEF of selected from equal to or above 70%, equal to or above 50% to equal to or below 70%, equal to or above 60%, equal to or below 60%, equal to or above 50% to equal to or below 60%, equal to or above 45%, equal to or below 45%, and equal to or above 40% to equal to or below 49%.

In one embodiment, the present disclosure includes treatment of a patient who has a LVEF of equal to or above 30%, 35%, 39%, 40%, 45%, 49%, 50%, 55%, 65% or 70%. In one embodiment, the patient has a LVEF equal to or above 30%. In one embodiment, the patient has a LVEF equal to or above 35%. In one embodiment, the patient has a LVEF equal to or above 39%. In one embodiment, the patient has a LVEF equal to or above 40%. In one embodiment, the patient has a LVEF equal to or above 45%. In one embodiment, the patient has a LVEF equal to or above 49%. In one embodiment, the patient has a LVEF equal to or above 50%. In one embodiment, the patient has a LVEF equal to or above 55%. In one embodiment, the patient has a LVEF equal to or above 59%. In one embodiment, the patient has a LVEF equal to or above 60%. In one embodiment, the patient has a LVEF equal to or above 65%. In one embodiment, the patient has a LVEF equal to or above 70%. In one embodiment, the patient has a LVEF equal to or above 75%.

In one embodiment, the patient has a LVEF equal to or below 30%, 35%, 39%, 40%, 45%, 49%, 50%, 55%, 65% or 70%. In one embodiment, the patient has a LVEF equal to or below 30%. In one embodiment, the patient has a LVEF equal to or below 35%. In one embodiment, the patient has a LVEF equal to or below 39%. In one embodiment, the patient has a LVEF equal to or below 40%. In one embodiment, the patient has a LVEF equal to or below 45%. In one embodiment, the patient has a LVEF equal to or below 49%. In one embodiment, the patient has a LVEF equal to or below 50%. In one embodiment, the patient has a LVEF equal to or below 55%. In one embodiment, the patient has a LVEF equal to or below 59%. In one embodiment, the patient has a LVEF equal to or below 60%. In one embodiment, the patient has a LVEF equal to or below 65%. In one embodiment, the patient has a LVEF equal to or below 70%. In one embodiment, the patient has a LVEF equal to or below 75%.

In one embodiment, the patient has a LVEF selected from 44±3%, equal to or above 41 to equal to or below 47%, equal to or above 41 to equal to or below 44%, equal to or above 44 to equal to or below 47%, 54±3%, equal to or above 51 to equal to or below 57%, equal to or above 51 to equal to or below 54%, equal to or above 54 to equal to or below 57%, 64±5%, equal to or above 59 to equal to or below 69%, equal to or above 59 to equal to or below 64%, equal to or above 64 to equal to or below 69%, 53 ±8%, equal to or above 45 to equal to or below 61%, equal to or above 45 to equal to or below 53%, and equal to or above 53 to equal to or below 61%.

In one embodiment, the patient has heart failure according to NYHA class II, III or IV, and a LVEF of equal to or above 30%, 35%, 39%, 40%, 45%, 49%, 50%, 55%, 65% or 70%. In one embodiment, the patient has heart failure according to NYHA class II, III or IV, and a LVEF of equal to or below 30%, 35%, 39%, 40%, 45%, 49%, 50%, 55%, 65% or 70%.

In one embodiment, the patient has heart failure according to NYHA class II, and a LVEF of equal to or above 30%, 35%, 39%, 40%, 45%, 49%, 50%, 55%, 60, 65% or 70%. In one embodiment, the patient has heart failure according to NYHA class II, and a LVEF of equal to or above 30%. In one embodiment, the patient has heart failure according to NYHA class II, and a LVEF equal to or above 35%. In one embodiment, the patient has heart failure according to NYHA class II, and a LVEF equal to or above 39%. In one embodiment, the patient has heart failure according to NYHA class II, and a LVEF of equal to or above 40%. In one embodiment, the patient has heart failure according to NYHA class II, and a LVEF of equal to or above 45%. In one embodiment, the patient has heart failure according to NYHA class II, and a LVEF of equal to or above 49%. In one embodiment, the patient has heart failure according to NYHA class II, and a LVEF of equal to or above 50%. In one embodiment, the patient has heart failure according to NYHA class II, and a LVEF of equal to or above 55%. In one embodiment, the patient has heart failure according to NYHA class II, and a LVEF of equal to or above 60%. In one embodiment, the patient has heart failure according to NYHA class II, and a LVEF of equal to or above 65%. In one embodiment, the patient has heart failure according to NYHA class II, and a LVEF of equal to or above 70%.

In one embodiment, the patient has heart failure according to NYHA class II, and a LVEF of equal to or below 30%, 35%, 39%, 40%, 45%, 49%, 50%, 55%, 60, 65% or 70%. In one embodiment, the patient has heart failure according to NYHA class II, and a LVEF of equal to or below 30%. In one embodiment, the patient has heart failure according to NYHA class II, and a LVEF of equal to or below 35%. In one embodiment, the patient has heart failure according to NYHA class II, and a LVEF of equal to or below 39%. In one embodiment, the patient has heart failure according to NYHA class II, and a LVEF of equal to or below 40%. In one embodiment, the patient has heart failure according to NYHA class II, and a LVEF of equal to or below 45%. In one embodiment, the patient has heart failure according to NYHA class II, and a LVEF of equal to or below 49%. In one embodiment, the patient has heart failure according to NYHA class II, and a LVEF of equal to or below 50%. In one embodiment, the patient has heart failure according to NYHA class II, and a LVEF of equal to or below 55%. In one embodiment, the patient has heart failure according to NYHA class II, and a LVEF of equal to or below 60%. In one embodiment, the patient has heart failure according to NYHA class II, and a LVEF of equal to or below 65%. In one embodiment, the patient has heart failure according to NYHA class II, and a LVEF of equal to or below 70%.

In one embodiment, the patient has heart failure according to NYHA class III, and a LVEF of equal to or above 30%, 35%, 39%, 40%, 45%, 49%, 50%, 55%, 60, 65% or 70%. In one embodiment, the patient has heart failure according to NYHA class III, and a LVEF of equal to or above 30%. In one embodiment, the patient has heart failure according to NYHA class III, and a LVEF of equal to or above 35%. In one embodiment, the patient has heart failure according to NYHA class III, and a LVEF of equal to or above 39%. In one embodiment, the patient has heart failure according to NYHA class III, and a LVEF of equal to or above 40%. In one embodiment, the patient has heart failure according to NYHA class III, and a LVEF of equal to or above 45%. In one embodiment, the patient has heart failure according to NYHA class III, and a LVEF of equal to or above 49%. In one embodiment, the patient has heart failure according to NYHA class III, and a LVEF of equal to or above 50%. In one embodiment, the patient has heart failure according to NYHA class III, and a LVEF of equal to or above 55%. In one embodiment, the patient has heart failure according to NYHA class III, and a LVEF of equal to or above 60%. In one embodiment, the patient has heart failure according to NYHA class III, and a LVEF of equal to or above 65%. In one embodiment, the patient has heart failure according to NYHA class III, and a LVEF of equal to or above 70%.

In one embodiment, the patient has heart failure according to NYHA class III, and a LVEF of equal to or below 30%, 35%, 39%, 40%, 45%, 49%, 50%, 55%, 60, 65% or 70%. In one embodiment, the patient has heart failure according to NYHA class III, and a LVEF of equal to or below 30%. In one embodiment, the patient has heart failure according to NYHA class III, and a LVEF of equal to or below 35%. In one embodiment, the patient has heart failure according to NYHA class III, and a LVEF of equal to or below 39%. In one embodiment, the patient has heart failure according to NYHA class III, and a LVEF of equal to or below 40%. In one embodiment, the patient has heart failure according to NYHA class III, and a LVEF of equal to or below 45%. In one embodiment, the patient has heart failure according to NYHA class III, and a LVEF of equal to or below 49%. In one embodiment, the patient has heart failure according to NYHA class III, and a LVEF of equal to or below 50%. In one embodiment, the patient has heart failure according to NYHA class III, and a LVEF of equal to or below 55%. In one embodiment, the patient has heart failure according to NYHA class III, and a LVEF of equal to or below 60%. In one embodiment, the patient has heart failure according to NYHA class III, and a LVEF of equal to or below 65%. In one embodiment, the patient has heart failure according to NYHA class III, and a LVEF of equal to or below 70%.

In one embodiment, the patient has heart failure according to NYHA class IV, and a LVEF of equal to or above 30%, 35%, 39%, 40%, 45%, 49%, 50%, 55%, 60, 65% or 70%. In one embodiment, the patient has heart failure according to NYHA class IV, and a LVEF of equal to or above 30%. In one embodiment, the patient has heart failure according to NYHA class IV, and a LVEF of equal to or above 35%. In one embodiment, the patient has heart failure according to NYHA class IV, and a LVEF of equal to or above 39%. In one embodiment, the patient has heart failure according to NYHA class IV, and a LVEF of equal to or above 40%. In one embodiment, the patient has heart failure according to NYHA class IV, and a LVEF of equal to or above 45%. In one embodiment, the patient has heart failure according to NYHA class IV, and a LVEF of equal to or above 49%. In one embodiment, the patient has heart failure according to NYHA class IV, and a LVEF of equal to or above 50%. In one embodiment, the patient has heart failure according to NYHA class IV, and a LVEF of equal to or above 55%. In one embodiment, the patient has heart failure according to NYHA class IV, and a LVEF of equal to or above 60%. In one embodiment, the patient has heart failure according to NYHA class IV, and a LVEF of equal to or above 65%. In one embodiment, the patient has heart failure according to NYHA class IV, and a LVEF of equal to or above 70%.

In one embodiment, the patient has heart failure according to NYHA class IV, and a LVEF of equal to or below 30%, 35%, 39%, 40%, 45%, 49%, 50%, 55%, 60, 65% or 70%. In one embodiment, the patient has heart failure according to NYHA class IV, and a LVEF of equal to or below 30%. In one embodiment, the patient has heart failure according to NYHA class IV, and a LVEF of equal to or below 35%. In one embodiment, the patient has heart failure according to NYHA class IV, and a LVEF of equal to or below 39%. In one embodiment, the patient has heart failure according to NYHA class IV, and a LVEF of equal to or below 40%. In one embodiment, the patient has heart failure according to NYHA class IV, and a LVEF of equal to or below 45%. In one embodiment, the patient has heart failure according to NYHA class IV, and a LVEF of equal to or below 49%. In one embodiment, the patient has heart failure according to NYHA class IV, and a LVEF of equal to or below 50%. In one embodiment, the patient has heart failure according to NYHA class IV, and a LVEF of equal to or below 55%. In one embodiment, the patient has heart failure according to NYHA class IV, and a LVEF of equal to or below 60%. In one embodiment, the patient has heart failure according to NYHA class IV, and a LVEF of equal to or below 65%. In one embodiment, the patient has heart failure according to NYHA class IV, and a LVEF of equal to or below 70%.

In one embodiment, the patient has heart failure according to
NYHA II and LVEF equal to or above 50% to equal to or below 60%
NYHA II and LVEF equal to or above 40% to equal to or below 60%,
NYHA II and LVEF equal to or above 40% to equal to or below 50%,
NYHA II and LVEF 50% to 70%,
NYHA II and LVEF 40% to 49%,
NYHA II and LVEF 30% to 39%,
NYHA III and LVEF equal to or above 50% to equal to or below 60%
NYHA III and LVEF equal to or above 40% to equal to or below 60%,
NYHA III and LVEF equal to or above 40% to equal to or below 50%,
NYHA III and LVEF 50% to 70%,
NYHA III and LVEF 40% to 49%,
NYHA III and LVEF 30% to 39%,
NYHA IV and LVEF equal to or above 50% to equal to or below 60%
NYHA IV and LVEF equal to or above 40% to equal to or below 60%,
NYHA IV and LVEF equal to or above 40% to equal to or below 50%,
NYHA IV and LVEF 50% to 70%,
NYHA IV and LVEF 40% to 49%, or
NYHA IV and LVEF 30% to 39%.

In one embodiment, the patient has an estimated glomerular filtration rate (eGFR) equal to or above 15 mL/min/1.73 m².

Glomerular filtration rate (GFR) is the volume of fluid filtered from the renal (kidney) glomerular capillaries into the Bowman's capsule per unit time. The GFR is typically recorded in units of volume per time, e.g., milliliters per minute (mL/min). eGFR is estimated GFR (eGFR) and is a mathematically derived entity based on a patient's serum creatinine level, age, sex and race. This is usually calculated by the laboratory analyzing the blood sample and reported along with the serum creatinine result. “Normal” GFR is approximately 100 but is often reported as >90 (“greater than 90”) or >60 (“greater than 60”). It is for this reason that patients (and some doctors) sometimes quote the eGFR as a percentage of normal kidney function. eGFR is expressed as mL/min/1.73 m².

In one embodiment, the patient has an eGFR of selected from equal to or above 15, 20, 25, 30, 35, 40, 45, 50, 55, 60, 65, 70, 75, 80, 85, and 90 mL/min/1.73 m². In one embodiment, the patient has an eGFR of equal to or above 15 mL/min/1.73 m². In one embodiment, the patient has an eGFR of equal to or above 20 mL/min/1.73 m². In one embodiment, the patient has an eGFR of equal to or above 25 mL/min/1.73 m². In one embodiment, the patient has an eGFR of equal to or above 30 mL/min/1.73 m². In one embodiment, the patient has an eGFR of equal to or above 35 mL/min/1.73 m². In one embodiment, the patient has an eGFR of equal to or above 40 mL/min/1.73 m². In one embodiment, the patient has an eGFR of equal to or above 45 mL/min/1.73 m². In one embodiment, the patient has an eGFR of equal to or above 50 mL/min/1.73 m². In one embodiment, the patient has an eGFR of equal to or above 55 mL/min/1.73 m². In one embodiment, the patient has an eGFR of equal to or above 60 mL/min/1.73 m². In one embodiment, the patient has an eGFR of equal to or above 65 mL/min/1.73 m². In one embodiment, the patient has an eGFR of equal to or above 70 mL/min/1.73 m². In one embodiment, the patient has an eGFR of equal to or above 75 mL/min/1.73 m². In one embodiment, the patient has an eGFR of equal to or above 80 mL/min/1.73 m². In one embodiment, the patient has an eGFR of equal to or above 85 mL/min/1.73 m². In one embodiment, the patient has an eGFR of equal to or above 90 mL/min/1.73 m².

In one embodiment, the patient has an eGFR of selected from equal to or below 15, 20, 25, 30, 35, 40, 45, 50, 55, 60, 65, 70, 75, 80, 85, and 90 mL/min/1.73 m². In one embodiment, the patient has an eGFR of equal to or below 15 mL/min/1.73 m². In one embodiment, the patient has an eGFR of equal to or below 20 mL/min/1.73 m². In one embodiment, the patient has an eGFR of equal to or below 25 mL/min/1.73 m². In one embodiment, the patient has an eGFR of equal to or below 30 mL/min/1.73 m². In one embodiment, the patient has an eGFR of equal to or below 35 mL/min/1.73 m². In one embodiment, the patient has an eGFR of equal to or below 40 mL/min/1.73 m². In one embodiment, the patient has an eGFR of equal to or below 45 mL/min/1.73 m². In one embodiment, the patient has an eGFR of equal to or below 50 mL/min/1.73 m². In one embodiment, the patient has an eGFR of equal to or below 55 mL/min/1.73 m². In one embodiment, the patient has an eGFR of equal to or below 60 mL/min/1.73 m². In one embodiment, the patient has an eGFR of equal to or below 65 mL/min/1.73 m². In one embodiment, the patient has an eGFR of equal to or below 70 mL/min/1.73 m². In one embodiment, the patient has an eGFR of equal to or below 75 mL/min/1.73 m². In one embodiment, the patient has an eGFR of equal to or below 80 mL/min/1.73 m². In one embodiment, the patient has an eGFR of equal to or below 85 mL/min/1.73 m². In one embodiment, the patient has an eGFR of equal to or below 90 mL/min/1.73 m².

In one embodiment, the patient has an eGFR of equal to or above 15 to equal to or below 90 mL/min/1.73 m². In one embodiment, the patient has an eGFR of equal to or above 15 to equal to or below 60 mL/min/1.73 m². In one embodiment, the patient has an eGFR of equal to or above 15 to equal to or below 45 mL/min/1.73 m². In one embodiment, the patient has an eGFR of equal to or above 15 to equal to or below 30 mL/min/1.73 m². In one embodiment, the patient has an eGFR of equal to or above 25 to equal to or below 90 mL/min/1.73 m². In one embodiment, the patient has an eGFR of equal to or above 25 to equal to or below 60 mL/min/1.73 m². In one embodiment, the patient has an eGFR of equal to or above 25 to equal to or below 45 mL/min/1.73 m². In one embodiment, the patient has an eGFR of equal to or above 25 to equal to or below 30 mL/min/1.73 m². In one embodiment, the patient has an eGFR of equal to or above 30 to equal to or below 90 mL/min/1.73 m². In one embodiment, the patient has an eGFR of equal to or above 30 to equal to or below 60 mL/min/1.73 m². In one embodiment, the patient has an eGFR of equal to or above 30 to equal to or below 45 mL/min/1.73 m². In one embodiment, the patient has an eGFR of equal to or above 45 to equal to or below 90 mL/min/1.73 m². In one embodiment, the patient has an eGFR of equal to or above 45 to equal to or below 60 mL/min/1.73 m². In one embodiment, the patient has an eGFR of equal to or above 60 to equal to or below 90 mL/min/1.73 m².

In one embodiment, the patient has an Urine Albumin to Creatinine Ratio (UACR) of equal to or above 5 mg/g.

The Urine Albumin to Creatinine Ratio is a result of measuring albumin in urine. The albumin concentration is not related to the urine volume, but to the creatinine concentration in the urine. The unit is “mg/g”. The usual ranges are as follows: “normal”: below 30 mg/g; “Microalbuminuria”: 30 to 300 mg/g; “Macroalbuminuria”: 300 to 3000 mg/g; “Major proteinuria”: above 3,000 mg/g. Albuminuria, as assessed via UACR estimations, serves as an early marker of cardiovascular and kidney disease as well as an important marker in predicting the risk for development and progression of HF.

In one embodiment, the patient has an UACR of equal to or above 10 mg/g. In one embodiment, the patient has an UACR of equal to or above 15 mg/g. In one embodiment, the patient has an UACR of equal to or above 20 mg/g. In one embodiment, the patient has an UACR of equal to or above 25 mg/g. In one embodiment, the patient has an UACR of equal to or above 30 mg/g. In one embodiment, the patient has an UACR of equal to or above 35 mg/g. In one embodiment, the patient has an UACR of equal to or above 40 mg/g. In one embodiment, the patient has an UACR of equal to or above 45 mg/g. In one embodiment, the patient has an UACR of equal to or above 50 mg/g. In one embodiment, the patient has an UACR of equal to or above 55 mg/g. In one embodiment, the patient has an UACR of equal to or above 60 mg/g. In one embodiment, the patient has an UACR of equal to or above 65 mg/g. In one embodiment, the patient has an UACR of equal to or above 70 mg/g. In one embodiment, the patient has an UACR of equal to or above 75 mg/g. In one embodiment, the patient has an UACR of equal to or above 80 mg/g. In one embodiment, the patient has an UACR of equal to or above 90 mg/g. In one embodiment, the patient has an UACR of equal to or above 95 mg/g. In one embodiment, the patient has an UACR of equal to or above 100 mg/g. In one embodiment, the patient has an UACR of equal to or above 105 mg/g. In one embodiment, the patient has an UACR of equal to or above 110 mg/g. In one embodiment, the patient has an UACR of equal to or above 125 mg/g. In one embodiment, the patient has an UACR of equal to or above 130 mg/g. In one embodiment, the patient has an UACR of equal to or above 135 mg/g. In one embodiment, the patient has an UACR of equal to or above 140 mg/g. In one embodiment, the patient has an UACR of equal to or above 145 mg/g. In one embodiment, the patient has an UACR of equal to or above 150 mg/g. In one embodiment, the patient has an UACR of equal to or above 155 mg/g. In one embodiment, the patient has an UACR of equal to or above 160 mg/g. In one embodiment, the patient has an UACR of equal to or above 165 mg/g. In one embodiment, the patient has an UACR of equal to or above 170 mg/g. In one embodiment, the patient has an UACR of equal to or above 175 mg/g. In one embodiment, the patient has an UACR of equal to or above 180 mg/g. In one embodiment, the patient has an UACR of equal to or above 185 mg/g. In one embodiment, the patient has an UACR of equal to or above 190 mg/g. In one embodiment, the patient has an UACR of equal to or above 195 mg/g. In one embodiment, the patient has an UACR of equal to or above 200 mg/g. In one embodiment, the patient has an UACR of equal to or above 205 mg/g. In one embodiment, the patient has an UACR of equal to or above 210 mg/g. In one embodiment, the patient has an UACR of equal to or above 225 mg/g. In one embodiment, the patient has an UACR of equal to or above 230 mg/g. In one embodiment, the patient has an UACR of equal to or above 235 mg/g. In one embodiment, the patient has an UACR of equal to or above 240 mg/g. In one embodiment, the patient has an UACR of equal to or above 245 mg/g. In one embodiment, the patient has an UACR of equal to or above 250 mg/g. In one embodiment, the patient has an UACR of equal to or above 255 mg/g. In one embodiment, the patient has an UACR of equal to or above 260 mg/g. In one embodiment, the patient has an UACR of equal to or above 265 mg/g. In one embodiment, the patient has an UACR of equal to or above 270 mg/g. In one embodiment, the patient has an UACR of equal to or above 275 mg/g. In one embodiment, the patient has an UACR of equal to or above 280 mg/g. In one embodiment, the patient has an UACR of equal to or above 285 mg/g. In one embodiment, the patient has an UACR of equal to or above 290 mg/g. In one embodiment, the patient has an UACR of equal to or above 295 mg/g. In one embodiment, the patient has an UACR of equal to or above 300 mg/g. In one embodiment, the patient has an UACR of equal to or above 305 mg/g. In one embodiment, the patient has an UACR of equal to or above 310 mg/g. In one embodiment, the patient has an UACR of equal to or above 325 mg/g. In one embodiment, the patient has an UACR of equal to or above 330 mg/g. In one embodiment, the patient has an UACR of equal to or above 335 mg/g. In one embodiment, the patient has an UACR of equal to or above 340 mg/g. In one embodiment, the patient has an UACR of equal to or above 345 mg/g. In one embodiment, the patient has an UACR of equal to or above 350 mg/g. In one embodiment, the patient has an UACR of equal to or above 355 mg/g. In one embodiment, the patient has an UACR 1of equal to or above 360 mg/g. In one embodiment, the patient has an UACR of equal to or above 365 mg/g. In one embodiment, the patient has an UACR of equal to or above 370 mg/g. In one embodiment, the patient has an UACR of equal to or above 375 mg/g. In one embodiment, the patient has an UACR of equal to or above 380 mg/g. In one embodiment, the patient has an UACR of equal to or above 385 mg/g. In one embodiment, the patient has an UACR of equal to or above 390 mg/g. In one embodiment, the patient has an UACR of equal to or above 395 mg/g. In one embodiment, the patient has an UACR of equal to or above 400 mg/g. In one embodiment, the patient has an UACR of equal to or above 405 mg/g. In one embodiment, the patient has an UACR of equal to or above 410 mg/g. In one embodiment, the patient has an UACR of equal to or above 415 mg/g. In one embodiment, the patient has an UACR of equal to or above 420 mg/g. In one embodiment, the patient has an UACR of equal to or above 425 mg/g. In one embodiment, the patient has an UACR of equal to or above 430 mg/g. In one embodiment, the patient has an UACR of equal to or above 435 mg/g. In one embodiment, the patient has an UACR of equal to or above 440 mg/g. In one embodiment, the patient has an UACR of equal to or above 445 mg/g. In one embodiment, the patient has an UACR of equal to or above 450 mg/g. In one embodiment, the patient has an UACR of equal to or above 455 mg/g. In one embodiment, the patient has an UACR of equal to or above 460 mg/g. In one embodiment, the patient has an UACR of equal to or above 465 mg/g. In one embodiment, the patient has an UACR of equal to or above 470 mg/g. In one embodiment, the patient has an UACR of equal to or above 475 mg/g. In one embodiment, the patient has an UACR of equal to or above 480 mg/g. In one embodiment, the patient has an UACR of equal to or above 485 mg/g. In one embodiment, the patient has an UACR of equal to or above 490 mg/g. In one embodiment, the patient has an UACR of equal to or above 495 mg/g. In one embodiment, the patient has an UACR of equal to or above 500 mg/g. In one embodiment, the patient has an UACR of equal to or above 505 mg/g. In one embodiment, the patient has an UACR of equal to or above 510 mg/g. In one embodiment, the patient has an UACR of equal to or above 515 mg/g. In one embodiment, the patient has an UACR of equal to or above 520 mg/g. In one embodiment, the patient has an UACR of equal to or above 525 mg/g. In one embodiment, the patient has an UACR of equal to or above 530 mg/g. In one embodiment, the patient has an UACR of equal to or above 535 mg/g. In one embodiment, the patient has an UACR of equal to or above 540 mg/g. In one embodiment, the patient has an UACR of equal to or above 550 mg/g. In one embodiment, the patient has an UACR of equal to or above 555 mg/g. In one embodiment, the patient has an UACR of equal to or above 560 mg/g. In one embodiment, the patient has an UACR of equal to or above 565 mg/g. In one embodiment, the patient has an UACR of equal to or above 570 mg/g. In one embodiment, the patient has an UACR of equal to or above 575 mg/g. In one embodiment, the patient has an UACR of equal to or above 580 mg/g. In one embodiment, the patient has an UACR of equal to or above 585 mg/g. In one embodiment, the patient has an UACR of equal to or above 590 mg/g. In one embodiment, the patient has an UACR of equal to or above 595 mg/g. In one embodiment, the patient has an UACR of equal to or above 600 mg/g. In one embodiment, the patient has an UACR of equal to or above 700 mg/g.

In one embodiment, the patient has an UACR of equal to or below 5 mg/g. In one embodiment, the patient has an UACR of equal to or below 10 mg/g. In one embodiment, the patient has an UACR of equal to or below 15 mg/g. In one embodiment, the patient has an UACR of equal to or below 20 mg/g. In one embodiment, the patient has an UACR of equal to or below 25 mg/g. In one embodiment, the patient has an UACR of equal to or below 30 mg/g. In one embodiment, the patient has an UACR of equal to or below 35 mg/g. In one embodiment, the patient has an UACR of equal to or below 40 mg/g. In one embodiment, the patient has an UACR of equal to or below 45 mg/g. In one embodiment, the patient has an UACR of equal to or below 50 mg/g. In one embodiment, the patient has an UACR of equal to or below 55 mg/g. In one embodiment, the patient has an UACR of equal to or below 60 mg/g. In one embodiment, the patient has an UACR of equal to or below 65 mg/g. In one embodiment, the patient has an UACR of equal to or below 70 mg/g. In one embodiment, the patient has an UACR of equal to or below 75 mg/g. In one embodiment, the patient has an UACR of equal to or below 80 mg/g. In one embodiment, the patient has an UACR of equal to or below 90 mg/g. In one embodiment, the patient has an UACR of equal to or below 95 mg/g. In one embodiment, the patient has an UACR of equal to or below 100 mg/g. In one embodiment, the patient has an UACR of equal to or below 105 mg/g. In one embodiment, the patient has an UACR of equal to or below 110 mg/g. In one embodiment, the patient has an UACR of equal to or below 125 mg/g. In one embodiment, the patient has an UACR of equal to or below 130 mg/g. In one embodiment, the patient has an UACR of equal to or below 135 mg/g. In one embodiment, the patient has an UACR of equal to or below 140 mg/g. In one embodiment, the patient has an UACR of equal to or below 145 mg/g. In one embodiment, the patient has an UACR of equal to or below 150 mg/g. In one embodiment, the patient has an UACR of equal to or below 155 mg/g. In one embodiment, the patient has an UACR of equal to or below 160 mg/g. In one embodiment, the patient has an UACR of equal to or below 165 mg/g. In one embodiment, the patient has an UACR of equal to or below 170 mg/g. In one embodiment, the patient has an UACR of equal to or below 175 mg/g. In one embodiment, the patient has an UACR of equal to or below 180 mg/g. In one embodiment, the patient has an UACR of equal to or below 185 mg/g. In one embodiment, the patient has an UACR of equal to or below 190 mg/g. In one embodiment, the patient has an UACR of equal to or below 195 mg/g. In one embodiment, the patient has an UACR of equal to or below 200 mg/g. In one embodiment, the patient has an UACR of equal to or below 205 mg/g. In one embodiment, the patient has an UACR of equal to or below 210 mg/g. In one embodiment, the patient has an UACR of equal to or below 225 mg/g. In one embodiment, the patient has an UACR of equal to or below 230 mg/g. In one embodiment, the patient has an UACR of equal to or below 235 mg/g. In one embodiment, the patient has an UACR of equal to or below 240 mg/g. In one embodiment, the patient has an UACR of equal to or below 245 mg/g. In one embodiment, the patient has an UACR of equal to or below 250 mg/g. In one embodiment, the patient has an UACR of equal to or below 255 mg/g. In one embodiment, the patient has an UACR of equal to or below 260 mg/g. In one embodiment, the patient has an UACR of equal to or below 265 mg/g. In one embodiment, the patient has an UACR of equal to or below 270 mg/g. In one embodiment, the patient has an UACR of equal to or below 275 mg/g. In one embodiment, the patient has an UACR of equal to or below 280 mg/g. In one embodiment, the patient has an UACR of equal to or below 285 mg/g. In one embodiment, the patient has an UACR of equal to or below 290 mg/g. In one embodiment, the patient has an UACR of equal to or below 295 mg/g. In one embodiment, the patient has an UACR of equal to or below 300 mg/g. In one embodiment, the patient has an UACR of equal to or below 305 mg/g. In one embodiment, the patient has an UACR of equal to or below 310 mg/g. In one embodiment, the patient has an UACR of equal to or below 315 mg/g. In one embodiment, the patient has an UACR of equal to or below 320 mg/g. In one embodiment, the patient has an UACR of equal to or below 325 mg/g. In one embodiment, the patient has an UACR of equal to or below 330 mg/g. In one embodiment, the patient has an UACR of equal to or below 335 mg/g. In one embodiment, the patient has an UACR of equal to or below 340 mg/g. In one embodiment, the patient has an UACR of equal to or below 345 mg/g. In one embodiment, the patient has an UACR of equal to or below 350 mg/g. In one embodiment, the patient has an UACR of equal to or below 355 mg/g. In one embodiment, the patient has an UACR of equal to or below 360 mg/g. In one embodiment, the patient has an UACR of equal to or below 365 mg/g. In one embodiment, the patient has an UACR of equal to or below 370 mg/g. In one embodiment, the patient has an UACR of equal to or below 375 mg/g. In one embodiment, the patient has an UACR of equal to or below 380 mg/g. In one embodiment, the patient has an UACR of equal to or below 385 mg/g. In one embodiment, the patient has an UACR of equal to or below 390 mg/g. In one embodiment, the patient has an UACR of equal to or below 395 mg/g. In one embodiment, the patient has an UACR of equal to or below 400 mg/g. In one embodiment, the patient has an UACR of equal to or below 405 mg/g. In one embodiment, the patient has an UACR of equal to or below 410 mg/g. In one embodiment, the patient has an UACR of equal to or below 425 mg/g. In one embodiment, the patient has an UACR of equal to or below 430 mg/g. In one embodiment, the patient has an UACR of equal to or below 435 mg/g. In one embodiment, the patient has an UACR of equal to or below 440 mg/g. In one embodiment, the patient has an UACR of equal to or below 445 mg/g. In one embodiment, the patient has an UACR of equal to or below 450 mg/g. In one embodiment, the patient has an UACR of equal to or below 455 mg/g. In one embodiment, the patient has an UACR of equal to or below 460 mg/g. In one embodiment, the patient has an UACR of equal to or below 465 mg/g. In one embodiment, the patient has an UACR of equal to or below 470 mg/g. In one embodiment, the patient has an UACR of equal to or below 475 mg/g. In one embodiment, the patient has an UACR of equal to or below 480 mg/g. In one embodiment, the patient has an UACR of equal to or below 485 mg/g. In one embodiment, the patient has an UACR of equal to or below 490 mg/g. In one embodiment, the patient has an UACR of equal to or below 495 mg/g. In one embodiment, the patient has an UACR of equal to or below 500 mg/g. In one embodiment, the patient has an UACR of equal to or below 505 mg/g. In one embodiment, the patient has an UACR of equal to or below 510 mg/g. In one embodiment, the patient has an UACR of equal to or below 515 mg/g. In one embodiment, the patient has an UACR of equal to or below 520 mg/g. In one embodiment, the patient has an UACR of equal to or below 525 mg/g. In one embodiment, the patient has an UACR of equal to or below 530 mg/g.

In one embodiment, the patient has an UACR of equal to or below 535 mg/g. In one embodiment, the patient has an UACR of equal to or below 540 mg/g. In one embodiment, the patient has an UACR of equal to or below 550 mg/g. In one embodiment, the patient has an UACR of equal to or below 555 mg/g. In one embodiment, the patient has an UACR of equal to or below 560 mg/g. In one embodiment, the patient has an UACR of equal to or below 565 mg/g. In one embodiment, the patient has an UACR of equal to or below 570 mg/g. In one embodiment, the patient has an UACR of equal to or below 575 mg/g. In one embodiment, the patient has an UACR of equal to or below 580 mg/g. In one embodiment, the patient has an UACR of equal to or below 585 mg/g. In one embodiment, the patient has an UACR of equal to or below 590 mg/g. In one embodiment, the patient has an UACR of equal to or below 595 mg/g. In one embodiment, the patient has an UACR of equal to or below 600 mg/g. In one embodiment, the patient has an UACR of equal to or below 700 mg/g. In one embodiment, the patient has an UACR of equal to or below 800 mg/g. In one embodiment, the patient has an UACR of equal to or below 900 mg/g. In one embodiment, the patient has an UACR of equal to or below 1000 mg/g. In one embodiment, the patient has an UACR of equal to or below 2000 mg/g. In one embodiment, the patient has an UACR of equal to or below 3000 mg/g. In one embodiment, the patient has an UACR of equal to or below 4000 mg/g. In one embodiment, the patient has an UACR of equal to or below 5000 mg/g.

In one embodiment, the patient has an UACR of equal to or above 5 to equal to or below 5000 mg/g. In one embodiment, the patient has an UACR of equal to or above 5 to equal to or below 4000 mg/g. In one embodiment, the patient has an UACR of equal to or above 5 to equal to or below 3000 mg/g. In one embodiment, the patient has an UACR of equal to or above 5 to equal to or below 2000 mg/g. In one embodiment, the patient has an UACR of equal to or above 5 to equal to or below 1000 mg/g. In one embodiment, the patient has an UACR of equal to or above 5 to equal to or below 500 mg/g. In one embodiment, the patient has an UACR of equal to or above 5 to equal to or below 400 mg/g. In one embodiment, the patient has an UACR of equal to or above 5 to equal to or below 300 mg/g. In one embodiment, the patient has an UACR of equal to or above 5 to equal to or below 200 mg/g. In one embodiment, the patient has an UACR of equal to or above 5 to equal to or below 100 mg/g. In one embodiment, the patient has an UACR of equal to or above 10 to equal to or below 5000 mg/g. In one embodiment, the patient has an UACR of equal to or above 10 to equal to or below 4000 mg/g. In one embodiment, the patient has an UACR of equal to or above 10 to equal to or below 3000 mg/g. In one embodiment, the patient has an UACR of equal to or above 10 to equal to or below 2000 mg/g. In one embodiment, the patient has an UACR of equal to or above 10 to equal to or below 1000 mg/g. In one embodiment, the patient has an UACR of equal to or above 10 to equal to or below 500 mg/g. In one embodiment, the patient has an UACR of equal to or above 10 to equal to or below 400 mg/g. In one embodiment, the patient has an UACR of equal to or above 10 to equal to or below 300 mg/g. In one embodiment, the patient has an UACR of equal to or above 10 to equal to or below 200 mg/g. In one embodiment, the patient has an UACR of equal to or above 10 to equal to or below 100 mg/g. In one embodiment, the patient has an UACR of equal to or above 100 to equal to or below 5000 mg/g. In one embodiment, the patient has an UACR of equal to or above 100 to equal to or below 4000 mg/g. In one embodiment, the patient has an UACR of equal to or above 100 to equal to or below 3000 mg/g. In one embodiment, the patient has an UACR of equal to or above 100 to equal to or below 2000 mg/g. In one embodiment, the patient has an UACR of equal to or above 100 to equal to or below 1000 mg/g. In one embodiment, the patient has an UACR of equal to or above 100 to equal to or below 500 mg/g. In one embodiment, the patient has an UACR of equal to or above 100 to equal to or below 400 mg/g. In one embodiment, the patient has an UACR of equal to or above 100 to equal to or below 300 mg/g. In one embodiment, the patient has an UACR of equal to or above 100 to equal to or below 200 mg/g. In one embodiment, the patient has an UACR of equal to or above 200 to equal to or below 5000 mg/g. In one embodiment, the patient has an UACR of equal to or above 200 to equal to or below 4000 mg/g. In one embodiment, the patient has an UACR of equal to or above 200 to equal to or below 3000 mg/g. In one embodiment, the patient has an UACR of equal to or above 200 to equal to or below 2000 mg/g. In one embodiment, the patient has an UACR of equal to or above 200 to equal to or below 1000 mg/g. In one embodiment, the patient has an UACR of equal to or above 200 to equal to or below 500 mg/g. In one embodiment, the patient has an UACR of equal to or above 200 to equal to or below 400 mg/g. In one embodiment, the patient has an UACR of equal to or above 200 to equal to or below 300 mg/g. In one embodiment, the patient has an UACR of equal to or above 300 to equal to or below 5000 mg/g. In one embodiment, the patient has an UACR of equal to or above 300 to equal to or below 4000 mg/g. In one embodiment, the patient has an UACR of equal to or above 300 to equal to or below 3000 mg/g. In one embodiment, the patient has an UACR of equal to or above 300 to equal to or below 2000 mg/g. In one embodiment, the patient has an UACR of equal to or above 300 to equal to or below 1000 mg/g. In one embodiment, the patient has an UACR of equal to or above 300 to equal to or below 500 mg/g. In one embodiment, the patient has an UACR of equal to or above 300 to equal to or below 400 mg/g. In one embodiment, the patient has an UACR of equal to or above 400 to equal to or below 5000 mg/g. In one embodiment, the patient has an UACR of equal to or above 400 to equal to or below 4000 mg/g. In one embodiment, the patient has an UACR of equal to or above 400 to equal to or below 3000 mg/g. In one embodiment, the patient has an UACR of equal to or above 400 to equal to or below 2000 mg/g. In one embodiment, the patient has an UACR of equal to or above 400 to equal to or below 1000 mg/g. In one embodiment, the patient has an UACR of equal to or above 400 to equal to or below 500 mg/g. In one embodiment, the patient has an UACR of equal to or above 500 to equal to or below 5000 mg/g. In one embodiment, the patient has an UACR of equal to or above 500 to equal to or below 4000 mg/g. In one embodiment, the patient has an UACR of equal to or above 500 to equal to or below 3000 mg/g. In one embodiment, the patient has an UACR of equal to or above 500 to equal to or below 2000 mg/g. In one embodiment, the patient has an UACR of equal to or above 500 to equal to or below 1000 mg/g. In one embodiment, the patient has an UACR of equal to or above 600 to equal to or below 5000 mg/g. In one embodiment, the patient has an UACR of equal to or above 600 to equal to or below 4000 mg/g. In one embodiment, the patient has an UACR of equal to or above 600 to equal to or below 3000 mg/g. In one embodiment, the patient has an UACR of equal to or above 600 to equal to or below 2000 mg/g. In one embodiment, the patient has an UACR of equal to or above 600 to equal to or below 1000 mg/g. In one embodiment, the patient has an UACR of equal to or above 700 to equal to or below 5000 mg/g. In one embodiment, the patient has an UACR of equal to or above 700 to equal to or below 4000 mg/g. In one embodiment, the patient has an UACR of equal to or above 700 to equal to or below 3000 mg/g. In one embodiment, the patient has an UACR of equal to or above 700 to equal to or below 2000 mg/g. In one embodiment, the patient has an UACR of equal to or above 700 to equal to or below 1000 mg/g. In one embodiment, the patient has an UACR of equal to or above 800 to equal to or below 5000 mg/g. In one embodiment, the patient has an UACR of equal to or above 800 to equal to or below 4000 mg/g. In one embodiment, the patient has an UACR of equal to or above 800 to equal to or below 3000 mg/g. In one embodiment, the patient has an UACR of equal to or above 800 to equal to or below 2000 mg/g. In one embodiment, the patient has an UACR of equal to or above 800 to equal to or below 1000 mg/g. In one embodiment, the patient has an UACR of equal to or above 900 to equal to or below 5000 mg/g. In one embodiment, the patient has an UACR of equal to or above 900 to equal to or below 4000 mg/g. In one embodiment, the patient has an UACR of equal to or above 900 to equal to or below 3000 mg/g. In one embodiment, the patient has an UACR of equal to or above 900 to equal to or below 2000 mg/g. In one embodiment, the patient has an UACR of equal to or above 900 to equal to or below 1000 mg/g. In one embodiment, the patient has an UACR of equal to or above 1000 to equal to or below 5000 mg/g. In one embodiment, the patient has an UACR of equal to or above 1000 to equal to or below 4000 mg/g. In one embodiment, the patient has an UACR of equal to or above 1000 to equal to or below 3000 mg/g. In one embodiment, the patient has an UACR of equal to or above 1000 to equal to or below 2000 mg/g. In one embodiment, the patient has an UACR of equal to or above 2000 to equal to or below 5000 mg/g. In one embodiment, the patient has an UACR of equal to or above 2000 to equal to or below 4000 mg/g. In one embodiment, the patient has an UACR of equal to or above 2000 to equal to or below 3000 mg/g. In one embodiment, the patient has an UACR of equal to or above 3000 to equal to or below 4000 mg/g. In one embodiment, the patient has an UACR of equal to or above 4000 to equal to or below 5000 mg/g.

In one embodiment, the patient has an UACR selected from of equal to or above 5, 10, 15, 20, 25, 30, 35, 40, 45, 50, 55, 60, 65, 70, 75, 80, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 550, 555, 560, 565, 570, 575, 580, 585, 590, 595 and 600 mg/g.

In one embodiment, the patient has an UACR selected from of equal to or below 5, 10, 15, 20, 25, 30, 35, 40, 45, 50, 55, 60, 65, 70, 75, 80, 90, 95, 100, 105, 110, 115, 120,125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320,325, 330, 335, 340, 345, 350, 355, 360, 365, 370, 375, 380, 385, 390, 395, 400, 405, 410, 415, 420,425, 430, 435, 440, 445, 450, 455, 460, 465, 470, 475, 480, 485, 490, 495, 500, 505, 510, 515, 520, 525, 530, 535, 540, 550, 555, 560, 565, 570, 575, 580, 585, 590, 595 and 600 mg/g.

In one embodiment, the patient has an UACR of equal to or above 5 to equal to or below 500 mg/g. In one embodiment, the patient has an UACR of equal to or above 5 to equal to or below 300 mg/g. In one embodiment, the patient has an UACR of equal to or above 5 to equal to or below 300 mg/g. In one embodiment, the patient has an UACR of equal to or above 5 to equal to or below 250 mg/g. In one embodiment, the patient has an UACR of equal to or above 5 to equal to or below 200 mg/g. In one embodiment, the patient has an UACR of equal to or above 5 to equal to or below 150 mg/g. In one embodiment, the patient has an UACR of equal to or above 5 to equal to or below 100 mg/g. In one embodiment, the patient has an UACR of equal to or above 5 to equal to or below 90 mg/g. In one embodiment, the patient has an UACR of equal to or above 5 to equal to or below 80 mg/g. In one embodiment, the patient has an UACR of equal to or above 5 to equal to or below 75 mg/g. In one embodiment, the patient has an UACR of equal to or above 5 to equal to or below 70 mg/g. [Ina: 65 mg/g?] In one embodiment, the patient has an UACR of equal to or above 5 to equal to or below 60 mg/g. In one embodiment, the patient has an UACR of equal to or above 5 to equal to or below 55 mg/g. In one embodiment, the patient has an UACR of equal to or above 10 to equal to or below 500 mg/g. In one embodiment, the patient has an UACR of equal to or above 10 to equal to or below 300 mg/g. In one embodiment, the patient has an UACR of equal to or above 10 to equal to or below 300 mg/g. In one embodiment, the patient has an UACR of equal to or above 10 to equal to or below 250 mg/g. In one embodiment, the patient has an UACR of equal to or above 10 to equal to or below 200 mg/g. In one embodiment, the patient has an UACR of equal to or above 10 to equal to or below 150 mg/g. In one embodiment, the patient has an UACR of equal to or above 10 to equal to or below 100 mg/g. In one embodiment, the patient has an UACR of equal to or above 10 to equal to or below 90 mg/g. In one embodiment, the patient has an UACR of equal to or above 10 to equal to or below 80 mg/g. In one embodiment, the patient has an UACR of equal to or above 10 to equal to or below 75 mg/g. In one embodiment, the patient has an UACR of equal to or above 10 to equal to or below 70 mg/g. [Ina: 65 mg/g?] In one embodiment, the patient has an UACR of equal to or above 10 to equal to or below 60 mg/g. In one embodiment, the patient has an UACR of equal to or above 10 to equal to or below 55 mg/g.

The patient can be characterized by KDIGO Risk Categories as described in The Kidney Disease: Improving Global Outcomes (KDIGO) Clinical Practice Guideline for the Management of Glomerular Diseases. KDIGO is the global nonprofit organization developing and implementing evidence-based clinical practice guidelines in kidney disease. Chronic Kidney Disease (CKD) is defined as abnormalities of kidney structure or function, present for more than 3 months, with implications for health. CKD is classified based on Cause, GFR category (G1: Normal or high; G2: Mildly decreased; G3a: Mildly to moderately decreased; G3b: Moderately to severely decreased; G4: Severely decreased; G5: Kidney failure), and Albuminuria category (A1: normal; A2: mildly; A3: Severely increased).

In one embodiment, the patient has an eGFR of equal to or below 15 mL/min/1.73 m² and a UACR of equal to or below 30 mg/g. In one embodiment, the patient has an eGFR of equal to or below 15 mL/min/1.73 m² and a UACR of equal to or above 30 to equal to or below 300 mg/g. In one embodiment, the patient has an eGFR of equal to or below 15 mL/min/1.73 m² and a UACR of equal to or above 300 mg/g. In one embodiment, the patient has an eGFR of equal to or above 15 to equal to or below 29 mL/min/1.73 m² and a UACR of equal to or below 30 mg/g. In one embodiment, the patient has an eGFR of equal to or above 15 to equal to or below 29 mL/min/1.73 m² and a UACR of equal to or above 30 to equal to or below 300 mg/g. In one embodiment, the patient has an eGFR equal to or above 15 to equal to or below 29 mL/min/1.73 m² and a UACR of equal to or below 300 mg/g. In one embodiment, the patient has an eGFR of equal to or above 15 to equal to or below 30 mL/min/1.73 m² and a UACR of equal to or below 30 mg/g. In one embodiment, the patient has an eGFR of equal to or above 15 to equal to or below 30 mL/min/1.73 m² and a UACR of equal to or above 30 to equal to or below 300 mg/g. In one embodiment, the patient has an eGFR equal to or above 15 to equal to or below 30 mL/min/1.73 m² and a UACR of equal to or below 300 mg/g. In one embodiment, the patient has an eGFR of equal to or above 30 to equal to or below 44 mL/min/1.73 m² and a UACR of equal to or below 30 mg/g. In one embodiment, the patient has an eGFR of equal to or above 30 to equal to or below 44 mL/min/1.73 m² and a UACR of equal to or above 30 to equal to or below 300 mg/g. In one embodiment, the patient has an eGFR of equal to or above 30 to equal to or below 44 mL/min/1.73 m² and a UACR of equal to or below 300 mg/g. In one embodiment, the patient has an eGFR of equal to or above 30 to equal to or below 45 mL/min/1.73 m² and a UACR of equal to or below 30 mg/g. In one embodiment, the patient has an eGFR of equal to or above 30 to equal to or below 45 mL/min/1.73 m² and a UACR of equal to or above 30 to equal to or below 300 mg/g. In one embodiment, the patient has an eGFR of equal to or above 30 to equal to or below 45 mL/min/1.73 m² and a UACR of equal to or below 300 mg/g. In one embodiment, the patient has an eGFR of equal to or above 45 to equal to or below 59 mL/min/1.73 m² and a UACR of equal to or below 30 mg/g. In one embodiment, the patient has an eGFR of equal to or above 45 to equal to or below 59 mL/min/1.73 m² and a UACR of equal to or above 30 to equal to or below 300 mg/g. In one embodiment, the patient has an eGFR of equal to or above 45 to equal to or below 59 mL/min/1.73 m² and a UACR of equal to or below 300 mg/g. In one embodiment, the patient has an eGFR of equal to or above 45 to equal to or below 60 mL/min/1.73 m² and a UACR of equal to or below 30 mg/g. In one embodiment, the patient has an eGFR of equal to or above 45 to equal to or below 60 mL/min/1.73 m² and a UACR of equal to or above 30 to equal to or below 300 mg/g. In one embodiment, the patient has an eGFR of equal to or above 45 to equal to or below 60 mL/min/1.73 m² and a UACR of equal to or below 300 mg/g. In one embodiment, the patient has an eGFR of equal to or above 60 to equal to or below 89 mL/min/1.73 m² and a UACR of equal to or below 30 mg/g. In one embodiment, the patient has an eGFR of equal to or above 60 to equal to or below 89 mL/min/1.73 m² and a UACR of equal to or above 30 to equal to or below 300 mg/g. In one embodiment, the patient has an eGFR of equal to or above 60 to equal to or below 89 mL/min/1.73 m² and a UACR of equal to or below 300 mg/g. In one embodiment, the patient has an eGFR of equal to or above 60 to equal to or below 90 mL/min/1.73 m² and a UACR of equal to or below 30 mg/g. In one embodiment, the patient has an eGFR of equal to or above 60 to equal to or below 90 mL/min/1.73 m² and a UACR of equal to or above 30 to equal to or below 300 mg/g. In one embodiment, the patient has an eGFR of equal to or above 60 to equal to or below 90 mL/min/1.73 m² and a UACR of equal to or below 300 mg/g. In one embodiment, the patient has an eGFR of equal to or above 90 mL/min/1.73 m² and a UACR of equal to or below 30 mg/g. In one embodiment, the patient has an eGFR of equal to or above 90 mL/min/1.73 m² and a UACR of equal to or above 30 to equal to or below 300 mg/g. In one embodiment, the patient has an eGFR of equal to or above 90 mL/min/1.73 m² and a UACR of equal to or below 300 mg/g.

The patient can also be characterized by potassium level expressed in “mmol/L”. The potassium level and distribution among the body compartments depend on a complex interplay of multiple factors including renal and gastrointestinal function; diet, medications, and supplements; neurohormonal status; and acid-base balance. Under normal conditions, the kidneys are responsible for up to 90-95% of potassium elimination, with the colon being responsible for the remainder. Because cardiac repolarization relies on potassium influx, hypokalemia lengthens the cardiac action potential and increases QT dispersion. Hyperkalemia leads to a shortening of the repolarization time, which may lead to QT interval shortening. Both hypo- and hyperkalemia may be life-threatening conditions by increasing the risk of ventricular arrhythmia and sudden cardiac death. Dyskalemia in HF has important prognostic implications. Critical comorbidities include CKD, diabetes mellitus (DM), frailty, and aging. In HF, as in other conditions, for example, myocardial infarction, hypertension, kidney disease, or in the general population, the relationship between diabetes mellitus concentrations and adverse outcomes appears to be U-shaped, where both low- and high-potassium levels are associated with adverse outcomes, although it remains unclear to what extent dyskalemia is a risk factor itself versus a risk marker representing the patients' overall clinical status, other comorbidities, and/or use or nonuse of HF medications. Correction of both hypokalemia and hyperkalemia offsets their associated risks.

The skilled artisan understands that the potassium test measures the amount of potassium in blood serum. He also knows how to measure serum potassium levels in both patients (especially those with conditions like heart failure and kidney disease) and healthy individuals according to the following steps and methods typically used (R. A. Garcia et al., J Clin Lab Anal. 2018 Jun. 19; 32 (9): e22594. doi: 10.1002/jcla.22594):
Sample Collection:
   Venipuncture: Blood is drawn from a vein, usually in the arm, using sterile techniques to prevent contamination.
   Sample Type: Serum is typically used for potassium measurement. After collection, the blood sample is allowed to clot, and then it is centrifuged to separate the serum from the blood cells.
Laboratory Methods:
   Ion-Selective Electrode (ISE): This is the most common method for measuring serum potassium. It uses a specific electrode that responds to potassium ions in the sample, providing a direct measurement.
   Flame Photometry: This method measures the concentration of potassium by passing the sample through a flame and detecting the emitted light at a specific wavelength.
   Colorimetric Methods: These involve chemical reactions that produce a color change proportional to the potassium concentration, which can be measured spectrophotometrically.

Normal serum potassium levels typically range from 3.5 to 5.0 mmol/L. Values outside this range may indicate hyperkalemia (high potassium) or hypokalemia (low potassium), which can have significant clinical implications, especially in patients with underlying conditions. For patients with conditions such as heart failure or kidney disease, regular monitoring is crucial to manage and adjust treatments effectively.

In one embodiment, the patient has a potassium level of above 3 mmol/L. In one embodiment, the patient has a potassium level of above 3.5 mmol/L. In one embodiment, the patient has a potassium level of above 4 mmol/L. In one embodiment, the patient has a potassium level of above 4.5 mmol/L. In one embodiment, the patient has a potassium level of above 4.8 mmol/L. In one embodiment, the patient has a potassium level of above 5 mmol/L. In one embodiment, the patient has a potassium level of above 5.5 mmol/L. In one embodiment, the patient has a potassium level of above 6 mmol/L. In one embodiment, the patient has a potassium level of above 6.5 mmol/L. In one embodiment, the patient has a potassium level of above 7 mmol/L. In one embodiment, the patient has a potassium level of above 7.5 mmol/L. In one embodiment, the patient has a potassium level equal to or above 3 mmol/L. In one embodiment, the patient has a potassium level equal to or above 3.5 mmol/L. In one embodiment, the patient has a potassium level equal to or above 4 mmol/L. In one embodiment, the patient has a potassium level equal to or above 4.5 mmol/L. In one embodiment, the patient has a potassium level equal to or above 4.8 mmol/L. In one embodiment, the patient has a potassium level equal to or above 5 mmol/L. In one embodiment, the patient has a potassium level equal to or above 5.5 mmol/L. In one embodiment, the patient has a potassium level equal to or above 6 mmol/L. In one embodiment, the patient has a potassium level equal to or above 6.5 mmol/L. In one embodiment, the patient has a potassium level equal to or above 7 mmol/L. In one embodiment, the patient has a potassium level equal to or above 7.5 mmol/L. In one embodiment, the patient has a potassium level below 3 mmol/L. In one embodiment, the patient has a potassium level below 3.5 mmol/L. In one embodiment, the patient has a potassium level below 4 mmol/L. In one embodiment, the patient has a potassium level below 4.5 mmol/L. In one embodiment, the patient has a potassium level below 4.8 mmol/L. In one embodiment, the patient has a potassium level below 5 mmol/L. In one embodiment, the patient has a potassium level below 5.5 mmol/L. In one embodiment, the patient has a potassium level below 6 mmol/L. In one embodiment, the patient has a potassium level below 6.5 mmol/L. In one embodiment, the patient has a potassium level below 7 mmol/L. In one embodiment, the patient has a potassium level below 7.5 mmol/L. In one embodiment, the patient has a potassium level equal to or below 3 mmol/L. In one embodiment, the patient has a potassium level equal to or below 3.5 mmol/L. In one embodiment, the patient has a potassium level equal to or below 4 mmol/L. In one embodiment, the patient has a potassium level equal to or below 4.5 mmol/L. In one embodiment, the patient has a potassium level equal to or below 4.8 mmol/L. In one embodiment, the patient has a potassium level equal to or below 5 mmol/L. In one embodiment, the patient has a potassium level equal to or below 5.5 mmol/L. In one embodiment, the patient has a potassium level equal to or below 6 mmol/L. In one embodiment, the patient has a potassium level equal to or below 6.5 mmol/L. In one embodiment, the patient has a potassium level equal to or below 7 mmol/L. In one embodiment, the patient has a potassium level equal to or below 7.5 mmol/L.

In one embodiment, the patient has a potassium level selected from of above 3, 3.5, 4, 4.5, 4.8, 5, 5.5, 6, 6.5, 7 or 7.5 mmol/L. In one embodiment, the patient has a potassium level selected from of equal to or above 3, 3.5, 4, 4.5, 4.8, 5, 5.5, 6, 6.5, 7 or 7.5 mmol/L. In one embodiment, the patient has a potassium level selected from of below 3, 3.5, 4, 4.5, 4.8, 5, 5.5, 6, 6.5, 7 or 7.5 mmol/L. In one embodiment, the patient has a potassium level selected from of equal to or below 3, 3.5, 4, 4.5, 4.8, 5, 5.5, 6, 6.5, 7 or 7.5 mmol/L.

In one embodiment, the patient has a potassium level of equal to or above 3 to equal to or below 3.5 mmol/L. In one embodiment, the patient has a potassium level of equal to or above 3 to equal to or below 4 mmol/L. In one embodiment, the patient has a potassium level of equal to or above 3 to equal to or below 4.5 mmol/L. In one embodiment, the patient has a potassium level of equal to or above 3 to equal to or below 4.8 mmol/L. In one embodiment, the patient has a potassium level of equal to or above 3 to equal to or below 5 mmol/L. In one embodiment, the patient has a potassium level of equal to or above 3 to equal to or below 5.5 mmol/L. In one embodiment, the patient has a potassium level of equal to or above 3 to equal to or below 6 mmol/L. In one embodiment, the patient has a potassium level of equal to or above 3 to equal to or below 6.5 mmol/L. In one embodiment, the patient has a potassium level of equal to or above 3 to equal to or below 7 mmol/L. In one embodiment, the patient has a potassium level of equal to or above 3 to equal to or below 7.5 mmol/L. In one embodiment, the patient has a potassium level of equal to or above 3.5 to equal to or below 4 mmol/L. In one embodiment, the patient has a potassium level of equal to or above 3.5 to equal to or below 4.5 mmol/L. In one embodiment, the patient has a potassium level of equal to or above 3.5 to equal to or below 4.8 mmol/L. In one embodiment, the patient has a potassium level of equal to or above 3.5 to equal to or below 5 mmol/L. In one embodiment, the patient has a potassium level of equal to or above 3.5 to equal to or below 5.5 mmol/L. In one embodiment, the patient has a potassium level of equal to or above 3.5 to equal to or below 6 mmol/L. In one embodiment, the patient has a potassium level of equal to or above 3.5 to equal to or below 6.5 mmol/L. In one embodiment, the patient has a potassium level of equal to or above 3.5 to equal to or below 7 mmol/L. In one embodiment, the patient has a potassium level of equal to or above 3.5 to equal to or below 7.5 mmol/L. In one embodiment, the patient has a potassium level of equal to or above 4 to equal to or below 4.5 mmol/L. In one embodiment, the patient has a potassium level of equal to or above 4 to equal to or below 4.8 mmol/L. In one embodiment, the patient has a potassium level of equal to or above 4 to equal to or below 5 mmol/L. In one embodiment, the patient has a potassium level of equal to or above 4 to equal to or below 5.5 mmol/L. In one embodiment, the patient has a potassium level of equal to or above 4 to equal to or below 6 mmol/L. In one embodiment, the patient has a potassium level of equal to or above 4 to equal to or below 6.5 mmol/L. In one embodiment, the patient has a potassium level of equal to or above 4 to equal to or below 7 mmol/L. In one embodiment, the patient has a potassium level of equal to or above 4 to equal to or below 7.5 mmol/L. In one embodiment, the patient has a potassium level of equal to or above 4.5 to equal to or below 4.8 mmol/L. In one embodiment, the patient has a potassium level of equal to or above 4.5 to equal to or below 5 mmol/L. In one embodiment, the patient has a potassium level of equal to or above 4.5 to equal to or below 5.5 mmol/L. In one embodiment, the patient has a potassium level of equal to or above 4.5 to equal to or below 6 mmol/L. In one embodiment, the patient has a potassium level of equal to or above 4.5 to equal to or below 6.5 mmol/L. In one embodiment, the patient has a potassium level of equal to or above 4.5 to equal to or below 7 mmol/L. In one embodiment, the patient has a potassium level of equal to or above 4.5 to equal to or below 7.5 mmol/L. In one embodiment, the patient has a potassium level of equal to or above 4.8 to equal to or below 5 mmol/L. In one embodiment, the patient has a potassium level of equal to or above 4.8 to equal to or below 5.5 mmol/L. In one embodiment, the patient has a potassium level of equal to or above 4.8 to equal to or below 6 mmol/L. In one embodiment, the patient has a potassium level of equal to or above 4.8 to equal to or below 6.5 mmol/L. In one embodiment, the patient has a potassium level of equal to or above 4.8 to equal to or below 7 mmol/L. In one embodiment, the patient has a potassium level of equal to or above 4.8 to equal to or below 7.5 mmol/L. In one embodiment, the patient has a potassium level of equal to or above 5 to equal to or below 5.5 mmol/L. In one embodiment, the patient has a potassium level of equal to or above 5 to equal to or below 6 mmol/L. In one embodiment, the patient has a potassium level of equal to or above 5 to equal to or below 6.5 mmol/L. In one embodiment, the patient has a potassium level of equal to or above 5 to equal to or below 7 mmol/L. In one embodiment, the patient has a potassium level of equal to or above 5 to equal to or below 7.5 mmol/L. In one embodiment, the patient has a potassium level of equal to or above 5.5 to equal to or below 6 mmol/L. In one embodiment, the patient has a potassium level of equal to or above 5.5 to equal to or below 6.5 mmol/L. In one embodiment, the patient has a potassium level of equal to or above 5.5 to equal to or below 7 mmol/L. In one embodiment, the patient has a potassium level of equal to or above 5.5 to equal to or below 7.5 mmol/L. In one embodiment, the patient has a potassium level of equal to or above 6 to equal to or below 6.5 mmol/L. In one embodiment, the patient has a potassium level of equal to or above 6 to equal to or below 7 mmol/L. In one embodiment, the patient has a potassium level of equal to or above 6 to equal to or below 7.5 mmol/L. In one embodiment, the patient has a potassium level of equal to or above 6.5 to equal to or below 7 mmol/L. In one embodiment, the patient has a potassium level of equal to or above 6.5 to equal to or below 7.5 mmol/L. In one embodiment, the patient has a potassium level of equal to or above 7 to equal to or below 7.5 mmol/L.

Measurement of serum sodium is routine in assessing electrolyte, acid-base, and water balance, as well as renal function. The sodium level is expressed in mmol/L. The reference range for serum sodium is 135-147 mmol/L, although different assays establish their own reference ranges, which may differ slightly.

Sodium excretion via the urine (‘natriuresis’) is a particular therapeutic target for cardiovascular diseases. For example, neurohumoral stimulation by the renin-angiotensin-aldosterone system (RAAS) contributes to permanent sodium retention and an associated extracellular volume load in the development of chronic heart and kidney failure. In particular, neurohumoral stimulation by the renin-angiotensin-aldosterone system (RAAS) or elevation of sodium-glucose-transporter-2 (SGLT2) expression in the proximal tubule contributes to permanent sodium retention and an associated extracellular volume load in the development of chronic heart and kidney failure. The body is no longer able to perform effective natriuresis, especially when there is impaired kidney function, which is often associated with heart failure. Therefore, maintaining an effective natriuresis would be useful in treatment and/or prevention of cardiovascular and/or renal diseases.

The patient can be characterized by the sodium level.

In one embodiment, the patient has a sodium level selected from of equal to or above 100, 110, 115, 117, 120, 125, 130, 135, 138, 140, 141, 144, 145, 150, 154 and 155 mmol/L. In one embodiment, the patient has a sodium level selected from of equal to or below 100, 110, 115, 117, 120, 125, 130, 135, 138, 140, 144, 145, 150, 154 and 155 mmol/L.

In one embodiment, the patient has a sodium level of equal to or above 100 mmol/L. In one embodiment, the patient has a sodium level of equal to or above 110 mmol/L. In one embodiment, the patient has a sodium level of equal to or above 115 mmol/L. In one embodiment, the patient has a sodium level of equal to or above 120 mmol/L. In one embodiment, the patient has a sodium level of equal to or above 125 mmol/L. In one embodiment, the patient has a sodium level of equal to or above 130 mmol/L. In one embodiment, the patient has a sodium level of equal to or above 135 mmol/L. In one embodiment, the patient has a sodium level of equal to or above 138 mmol/L. In one embodiment, the patient has a sodium level of equal to or above 140 mmol/L. In one embodiment, the patient has a sodium level of equal to or above 144 mmol/L. In one embodiment, the patient has a sodium level of equal to or above 145 mmol/L. In one embodiment, the patient has a sodium level of equal to or above 150 mmol/L. In one embodiment, the patient has a sodium level of equal to or below 154 mmol/L. In one embodiment, the patient has a sodium level of equal to or above 155 mmol/L. In one embodiment, the patient has a sodium level of equal to or below 100 mmol/L. In one embodiment, the patient has a sodium level of equal to or below 110 mmol/L. In one embodiment, the patient has a sodium level of equal to or below 115 mmol/L. In one embodiment, the patient has a sodium level of equal to or below 120 mmol/L. In one embodiment, the patient has a sodium level of equal to or below 125 mmol/L. In one embodiment, the patient has a sodium level of equal to or below 130 mmol/L. In one embodiment, the patient has a sodium level of equal to or below 135 mmol/L. In one embodiment, the patient has a sodium level of equal to or below 138 mmol/L. In one embodiment, the patient has a sodium level of equal to or below 140 mmol/L. In one embodiment, the patient has a sodium level of equal to or below 144 mmol/L. In one embodiment, the patient has a sodium level of equal to or below 145 mmol/L. In one embodiment, the patient has a sodium level of equal to or below 150 mmol/L. In one embodiment, the patient has a sodium level of equal to or below 154 mmol/L. In one embodiment, the patient has a sodium level of equal to or below 155 mmol/L.

In one embodiment, the patient has a sodium level of equal to or above 100 to equal to or below 110 mmol/L. In one embodiment, the patient has a sodium level of equal to or above 100 to equal to or below 115 mmol/L. In one embodiment, the patient has a sodium level of equal to or above 100 to equal to or below 117 mmol/L. In one embodiment, the patient has a sodium level of equal to or above 100 to equal to or below 120 mmol/L. In one embodiment, the patient has a sodium level of equal to or above 100 to equal to or below 125 mmol/L. In one embodiment, the patient has a sodium level of equal to or above 100 to equal to or below 130 mmol/L. In one embodiment, the patient has a sodium level of equal to or above 100 to equal to or below 135 mmol/L. In one embodiment, the patient has a sodium level of equal to or above 100 to equal to or below 138 mmol/L. In one embodiment, the patient has a sodium level of equal to or above 100 to equal to or below 140 mmol/L. In one embodiment, the patient has a sodium level of equal to or above 100 to equal to or below 141 mmol/L. In one embodiment, the patient has a sodium level of equal to or above 100 to equal to or below 144 mmol/L. In one embodiment, the patient has a sodium level of equal to or above 100 to equal to or below 145 mmol/L. In one embodiment, the patient has a sodium level of equal to or above 100 to equal to or below 150 mmol/L. In one embodiment, the patient has a sodium level of equal to or above 100 to equal to or below 154 mmol/L. In one embodiment, the patient has a sodium level of equal to or above 100 to equal to or below 155 mmol/L. In one embodiment, the patient has a sodium level of equal to or above 110 to equal to or below 115 mmol/L. In one embodiment, the patient has a sodium level of equal to or above 110 to equal to or below 117 mmol/L. In one embodiment, the patient has a sodium level of equal to or above 110 to equal to or below 120 mmol/L. In one embodiment, the patient has a sodium level of equal to or above 110 to equal to or below 125 mmol/L. In one embodiment, the patient has a sodium level of equal to or above 110 to equal to or below 130 mmol/L. In one embodiment, the patient has a sodium level of equal to or above 110 to equal to or below 135 mmol/L. In one embodiment, the patient has a sodium level of equal to or above 110 to equal to or below 138 mmol/L. In one embodiment, the patient has a sodium level of equal to or above 110 to equal to or below 140 mmol/L. In one embodiment, the patient has a sodium level of equal to or above 110 to equal to or below 141 mmol/L. In one embodiment, the patient has a sodium level of equal to or above 110 to equal to or below 144 mmol/L. In one embodiment, the patient has a sodium level of equal to or above 110 to equal to or below 145 mmol/L. In one embodiment, the patient has a sodium level of equal to or above 110 to equal to or below 150 mmol/L. In one embodiment, the patient has a sodium level of equal to or above 110 to equal to or below 154 mmol/L. In one embodiment, the patient has a sodium level of equal to or above 110 to equal to or below 155 mmol/L. In one embodiment, the patient has a sodium level of equal to or above 117 to equal to or below 120 mmol/L. In one embodiment, the patient has a sodium level of equal to or above 117 to equal to or below 125 mmol/L. In one embodiment, the patient has a sodium level of equal to or above 117 to equal to or below 130 mmol/L. In one embodiment, the patient has a sodium level of equal to or above 117 to equal to or below 135 mmol/L. In one embodiment, the patient has a sodium level of equal to or above 117 to equal to or below 138 mmol/L. In one embodiment, the patient has a sodium level of equal to or above 117 to equal to or below 140 mmol/L. In one embodiment, the patient has a sodium level of equal to or above 117 to equal to or below 141 mmol/L. In one embodiment, the patient has a sodium level of equal to or above 117 to equal to or below 144 mmol/L. In one embodiment, the patient has a sodium level of equal to or above 117 to equal to or below 145 mmol/L. In one embodiment, the patient has a sodium level of equal to or above 117 to equal to or below 150 mmol/L. In one embodiment, the patient has a sodium level of equal to or above 117 to equal to or below 154 mmol/L. In one embodiment, the patient has a sodium level of equal to or above 117 to equal to or below 155 mmol/L. In one embodiment, the patient has a sodium level of equal to or above 120 to equal to or below 125 mmol/L. In one embodiment, the patient has a sodium level of equal to or above 120 to equal to or below 130 mmol/L. In one embodiment, the patient has a sodium level of equal to or above 120 to equal to or below 135 mmol/L. In one embodiment, the patient has a sodium level of equal to or above 120 to equal to or below 138 mmol/L. In one embodiment, the patient has a sodium level of equal to or above 120 to equal to or below 140 mmol/L. In one embodiment, the patient has a sodium level of equal to or above 120 to equal to or below 141 mmol/L. In one embodiment, the patient has a sodium level of equal to or above 120 to equal to or below 144 mmol/L. In one embodiment, the patient has a sodium level of equal to or above 120 to equal to or below 145 mmol/L. In one embodiment, the patient has a sodium level of equal to or above 120 to equal to or below 150 mmol/L. In one embodiment, the patient has a sodium level of equal to or above 120 to equal to or below 154 mmol/L. In one embodiment, the patient has a sodium level of equal to or above 120 to equal to or below 155 mmol/L. In one embodiment, the patient has a sodium level of equal to or above 125 to equal to or below 130 mmol/L. In one embodiment, the patient has a sodium level of equal to or above 125 to equal to or below 135 mmol/L. In one embodiment, the patient has a sodium level of equal to or above 125 to equal to or below 138 mmol/L. In one embodiment, the patient has a sodium level of equal to or above 125 to equal to or below 140 mmol/L. In one embodiment, the patient has a sodium level of equal to or above 125 to equal to or below 141 mmol/L. In one embodiment, the patient has a sodium level of equal to or above 125 to equal to or below 144 mmol/L. In one embodiment, the patient has a sodium level of equal to or above 125 to equal to or below 145 mmol/L. In one embodiment, the patient has a sodium level of equal to or above 125 to equal to or below 150 mmol/L. In one embodiment, the patient has a sodium level of equal to or above 125 to equal to or below 154 mmol/L. In one embodiment, the patient has a sodium level of equal to or above 125 to equal to or below 155 mmol/L. In one embodiment, the patient has a sodium level of equal to or above 130 to equal to or below 135 mmol/L. In one embodiment, the patient has a sodium level of equal to or above 130 to equal to or below 138 mmol/L. In one embodiment, the patient has a sodium level of equal to or above 130 to equal to or below 140 mmol/L. In one embodiment, the patient has a sodium level of equal to or above 130 to equal to or below 141 mmol/L. In one embodiment, the patient has a sodium level of equal to or above 130 to equal to or below 144 mmol/L. In one embodiment, the patient has a sodium level of equal to or above 130 to equal to or below 145 mmol/L. In one embodiment, the patient has a sodium level of equal to or above 130 to equal to or below 150 mmol/L. In one embodiment, the patient has a sodium level of equal to or above 130 to equal to or below 154 mmol/L. In one embodiment, the patient has a sodium level of equal to or above 130 to equal to or below 155 mmol/L. In one embodiment, the patient has a sodium level of equal to or above 135 to equal to or below 138 mmol/L. In one embodiment, the patient has a sodium level of equal to or above 135 to equal to or below 140 mmol/L. In one embodiment, the patient has a sodium level of equal to or above 135 to equal to or below 141 mmol/L. In one embodiment, the patient has a sodium level of equal to or above 135 to equal to or below 144 mmol/L. In one embodiment, the patient has a sodium level of equal to or above 135 to equal to or below 145 mmol/L. In one embodiment, the patient has a sodium level of equal to or above 135 to equal to or below 150 mmol/L. In one embodiment, the patient has a sodium level of equal to or above 135 to equal to or below 154 mmol/L. In one embodiment, the patient has a sodium level of equal to or above 135 to equal to or below 155 mmol/L. In one embodiment, the patient has a sodium level of equal to or above 138 to equal to or below 140 mmol/L. In one embodiment, the patient has a sodium level of equal to or above 138 to equal to or below 141 mmol/L. In one embodiment, the patient has a sodium level of equal to or above 138 to equal to or below 144 mmol/L. In one embodiment, the patient has a sodium level of equal to or above 138 to equal to or below 145 mmol/L. In one embodiment, the patient has a sodium level of equal to or above 138 to equal to or below 150 mmol/L. In one embodiment, the patient has a sodium level of equal to or above 138 to equal to or below 154 mmol/L. In one embodiment, the patient has a sodium level of equal to or above 138 to equal to or below 155 mmol/L. In one embodiment, the patient has a sodium level of equal to or above 140 to equal to or below 141 mmol/L. In one embodiment, the patient has a sodium level of equal to or above 140 to equal to or below 144 mmol/L. In one embodiment, the patient has a sodium level of equal to or above 140 to equal to or below 145 mmol/L. In one embodiment, the patient has a sodium level of equal to or above 140 to equal to or below 150 mmol/L. In one embodiment, the patient has a sodium level of equal to or above 140 to equal to or below 154 mmol/L. In one embodiment, the patient has a sodium level of equal to or above 140 to equal to or below 155 mmol/L. In one embodiment, the patient has a sodium level of equal to or above 141 to equal to or below 144 mmol/L. In one embodiment, the patient has a sodium level of equal to or above 141 to equal to or below 145 mmol/L. In one embodiment, the patient has a sodium level of equal to or above 141 to equal to or below 150 mmol/L. In one embodiment, the patient has a sodium level of equal to or above 141 to equal to or below 154 mmol/L. In one embodiment, the patient has a sodium level of equal to or above 141 to equal to or below 155 mmol/L. In one embodiment, the patient has a sodium level of equal to or above 144 to equal to or below 145 mmol/L. In one embodiment, the patient has a sodium level of equal to or above 144 to equal to or below 150 mmol/L. In one embodiment, the patient has a sodium level of equal to or above 144 to equal to or below 154 mmol/L. In one embodiment, the patient has a sodium level of equal to or above 144 to equal to or below 155 mmol/L. In one embodiment, the patient has a sodium level of equal to or above 145 to equal to or below 150 mmol/L. In one embodiment, the patient has a sodium level of equal to or above 145 to equal to or below 154 mmol/L. In one embodiment, the patient has a sodium level of equal to or above 145 to equal to or below 155 mmol/L. In one embodiment, the patient has a sodium level of equal to or above 150 to equal to or below 154 mmol/L. In one embodiment, the patient has a sodium level of equal to or above 150 to equal to or below 155 mmol/L. In one embodiment, the patient has a sodium level of equal to or above 154 to equal to or below 155 mmol/L.

The chloride level can be used as a prognostic marker in HF. Chloride is among the major electrolytes that play a unique role in fluid homeostasis and is associated with cardiorenal and neurohormonal systems. Hypochloremia (low serum chloride level) can be an independent predictor of adverse outcomes in acute or chronic HF. Various HF therapies may cause hypochloremia, and hypochloremia itself can initiate and exacerbate diuretic resistance in HF. The chloride concentration is also used to calculate the anion gap, the difference between the main cation sodium and the two main anions chloride and bicarbonate. The chloride level is expressed in mmol/L.

Lower chloride levels are associated with a worse prognosis in HF. Further, low chloride levels may have a prominent contribution to the pathophysiology. Chloride, rather than sodium, modulates the renin secretion and tubuloglomerular feedback in the kidney and regulates sodium transport pathway in the Loop of Henle and distal convoluted tube. The relationship between chloride levels and prognosis was observed in both HF with reduced ejection fraction (HFrEF) and HF with mildly reduced or preserved ejection fraction (HFmrEF/HFpEF), and cam have a contribution to the pathophysiology and prognosis in HF. Higher chloride levels may potentially alleviate hypochloremia-related HF symptoms and prognosis. In one embodiment, the patient has a chloride level selected from of equal to or above 90, 95, 100, 105, 110 mmol/L. In one embodiment, the patient has a chloride level selected from of equal to or below 90, 95, 100, 105, 110 mmol/L.

In one embodiment, the patient has a chloride level of equal to or below 90 mmol/L. In one embodiment, the patient has a chloride level of equal to or below 95 mmol/L. In one embodiment, the patient has a chloride level of equal to or below 100 mmol/L. In one embodiment, the patient has a chloride level of equal to or below 105 mmol/L. In one embodiment, the patient has a chloride level of equal to or below 110 mmol/L. In one embodiment, the patient has a chloride level of equal to or above 90 mmol/L. In one embodiment, the patient has a chloride level of equal to or above 95 mmol/L. In one embodiment, the patient has a chloride level of equal to or above 100 mmol/L. In one embodiment, the patient has a chloride level of equal to or above 105 mmol/L. In one embodiment, the patient has a chloride level of equal to or above 110 mmol/L.

In one embodiment, the patient has a chloride level of equal to or above 90 to equal to or below 110 mmol/L. In one embodiment, the patient has a chloride level of equal to or above 90 to equal to or below 105 mmol/L. In one embodiment, the patient has a chloride level of equal to or above 90 to equal to or below 100 mmol/L. In one embodiment, the patient has a chloride level of equal to or above 90 to equal to or below 95 mmol/L.

Blood urea refers to the concentration of urea, a waste product produced when proteins break down in the bloodstream. It is a frequently measured parameter in clinical settings to evaluate the overall health and function of the kidneys. The kidneys remove urea from the blood after it is created in the liver as a byproduct of protein breakdown. Blood urea levels offer important information about kidney function. The ranges can vary slightly depending on age, gender, and health status or depending on the lab performing the test. The urea level in serum is expressed in mg/dL.

In one embodiment the patient has a urea level of equal to or above 6 to equal to or below 24 mg/dL. In one embodiment the patient has a urea level of equal to or above 6 to equal to or below 21 mg/dL. In one embodiment the patient has a urea level of equal to or above 6 mg/dL. In one embodiment the patient has a urea level of equal to or below 6 mg/dL. In one embodiment the patient has a urea level of equal to or above 21 mg/dL. In one embodiment the patient has a urea level of equal to or below 21 mg/dL. In one embodiment the patient has a urea level of equal to or above 24 mg/dL. In one embodiment the patient has a urea level of equal to or below 24 mg/dL.

In one embodiment, the patient has a urea level selected from of equal to or below 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 48, 49, 50, 55, 60, 65, 70, 75, 80, 85, 90, 94, 95 and 100 mg/dL. In one embodiment, the patient has a urea level selected from of equal to or above 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 48, 49, 50, 55, 60, 65, 70, 75, 80, 85, 90, 94, 95 and 100 mg/dL. In one embodiment the patient has a urea level of equal to or above 4 to equal to or below 94 mg/dL.

In one embodiment the patient has a urea level of equal to or above 4 mg/dL. In one embodiment the patient has a urea level of equal to or above 6 mg/dL. In one embodiment the patient has a urea level of equal to or above 8 mg/dL. In one embodiment the patient has a urea level of equal to or above 13 mg/dL. In one embodiment the patient has a urea level of equal to or above 16 mg/dL. In one embodiment the patient has a urea level of equal to or above 20 mg/dL. In one embodiment the patient has a urea level of equal to or above 23 mg/dL. In one embodiment the patient has a urea level of equal to or above 27 mg/dL. In one embodiment the patient has a urea level of equal to or above 33 mg/dL. In one embodiment the patient has a urea level of equal to or above 35 mg/dL. In one embodiment the patient has a urea level of equal to or above 40 mg/dL. In one embodiment the patient has a urea level of equal to or above 45 mg/dL. In one embodiment the patient has a urea level of equal to or above 50 mg/dL. In one embodiment the patient has a urea level of equal to or above 55 mg/dL. In one embodiment the patient has a urea level of equal to or above 60 mg/dL. In one embodiment the patient has a urea level of equal to or above 65 mg/dL. In one embodiment the patient has a urea level of equal to or above 70 mg/dL. In one embodiment the patient has a urea level of equal to or above 75 mg/dL. In one embodiment the patient has a urea level of equal to or above 80 mg/dL. In one embodiment the patient has a urea level of equal to or above 85 mg/dL. In one embodiment the patient has a urea level of equal to or above 90 mg/dL. In one embodiment the patient has a urea level of equal to or above 94 mg/dL. In one embodiment the patient has a urea level of equal to or above 95 mg/dL.

In one embodiment the patient has a urea level of equal to or below 4 mg/dL. In one embodiment the patient has a urea level of equal to or below 6 mg/dL. In one embodiment the patient has a urea level of equal to or below 8 mg/dL. In one embodiment the patient has a urea level of equal to or below 13 mg/dL. In one embodiment the patient has a urea level of equal to or below 16 mg/dL. In one embodiment the patient has a urea level of equal to or below 20 mg/dL. In one embodiment the patient has a urea level of equal to or below 23 mg/dL. In one embodiment the patient has a urea level of equal to or below 27 mg/dL. In one embodiment the patient has a urea level of equal to or below 33 mg/dL. In one embodiment the patient has a urea level of equal to or below 35 mg/dL. In one embodiment the patient has a urea level of equal to or below 40 mg/dL. In one embodiment the patient has a urea level of equal to or below 45 mg/dL. In one embodiment the patient has a urea level of equal to or below 50 mg/dL. In one embodiment the patient has a urea level of equal to or below 55 mg/dL. In one embodiment the patient has a urea level of equal to or below 60 mg/dL. In one embodiment the patient has a urea level of equal to or below 65 mg/dL. In one embodiment the patient has a urea level of equal to or below 70 mg/dL. In one embodiment the patient has a urea level of equal to or below 75 mg/dL. In one embodiment the patient has a urea level of equal to or below 80 mg/dL. In one embodiment the patient has a urea level of equal to or below 85 mg/dL. In one embodiment the patient has a urea level of equal to or below 90 mg/dL. In one embodiment the patient has a urea level of equal to or below 94 mg/dL. In one embodiment the patient has a urea level of equal to or below 95 mg/dL.

Blood urea nitrogen (BUN) is a medical test that measures the amount of urea nitrogen found in blood. The test for measuring urea in the blood is similar worldwide, but the results are reported in two different ways depending on the country doing the lab testing. In some instances, the urea level in plasma or serum is measured as nitrogen and is called “Blood Urea Nitrogen” or BUN. The unit used for BUN is mg/dL. In other instances, the whole urea molecule is measured, not just the nitrogen, and is reported in Standard International units of mmol/L. This measurement is about twice as high as the BUN measurement because BUN only measures the nitrogen part of the molecule (having a Molecular Weight of 28), while urea measures the whole molecule (having a Molecular Weight of 60). Thus, urea is approximately twice that of BUN (60/28=2.14). For example, a BUN result of 10 mg/dL is equal to a urea result of 21.4 mg/dL. Converting BUN (mg/dl) to Urea (mmol/L). The liver produces urea in the urea cycle as a waste product of the digestion of protein. Normal human adult blood should contain 6 to 20 mg/dl (2.1 to 7.1 mmol/L) of urea nitrogen. Individual laboratories will have different reference ranges, as the assay used can vary between laboratories. The test is used to detect renal problems. It is not considered as reliable as creatinine or BUN/creatinine ratio blood studies.

Troponin is a protein found in the body, specifically in heart muscle cells. The three main types of cardiac troponin proteins are I, T, and C. During, e.g., a heart attack, troponin spills into the bloodstream and it is a biomarker that can indicate cardiac injury. The high-sensitivity cardiac troponin test allows for detection of very low levels of troponin T, helping to diagnose, e.g., heart attacks, diagnose other heart-related conditions, Obstructive coronary artery disease (CAD), stable angina, congestive heart failure (CHF), cardiomyopathy and the like. Since the first use of troponin testing, several generations of more refined and more reliable tests have been developed and used to help diagnose heart attack more rapidly and accurately.

Assessed values of hs-Troponin T (high-sensitivity Cardiac Troponin T) in patients with chronic heart failure have recently emerged as an independent predictor of all cause mortality, cardiovascular mortality as well as hospitalization for cardiovascular causes. The prognostic value of hs-Troponin T is unaffected by heart failure etiology. (Aimo et al. Prognostic value of high-sensitivity Troponin-T in chronic heart failure: an individual patient data meta-analysis. Circulation. 2018; 137:286-297. doi: 10.1161/CIRCULATIONAHA.117.031560.). High-sensitivity Troponin T is expressed in ng/L.

In one embodiment, the patient has a High-sensitivity Troponin T level from equal to or above 5 to equal to or below 95000 ng/L. In one embodiment, the patient has a High-sensitivity Troponin T level from equal to or above 5 to equal to or below 9500 ng/L. In one embodiment, the patient has a High-sensitivity Troponin T level from equal to or above 5 to equal to or below 8000 ng/L. In one embodiment, the patient has a High-sensitivity Troponin T level from equal to or above 5 to equal to or below 7000 ng/L. In one embodiment, the patient has a High-sensitivity Troponin T level from equal to or above 5 to equal to or below 6000 ng/L. In one embodiment, the patient has a High-sensitivity Troponin T level from equal to or above 5 to equal to or below 5000 ng/L. In one embodiment, the patient has a High-sensitivity Troponin T level from equal to or above 5 to equal to or below 4000 ng/L. In one embodiment, the patient has a High-sensitivity Troponin T level from equal to or above 5 to equal to or below 3000 ng/L. In one embodiment, the patient has a High-sensitivity Troponin T level from equal to or above 5 to equal to or below 2000 ng/L. In one embodiment, the patient has a High-sensitivity Troponin T level from equal to or above 5 to equal to or below 1000 ng/L. In one embodiment, the patient has a High-sensitivity Troponin T level from equal to or above 5 to equal to or below 900 ng/L. In one embodiment, the patient has a High-sensitivity Troponin T level from equal to or above 5 to equal to or below 800 ng/L. In one embodiment, the patient has a High-sensitivity Troponin T level from equal to or above 5 to equal to or below 700 ng/L. In one embodiment, the patient has a High-sensitivity Troponin T level from equal to or above 5 to equal to or below 600 ng/L. In one embodiment, the patient has a High-sensitivity Troponin T level from equal to or above 5 to equal to or below 500 ng/L. In one embodiment, the patient has a High-sensitivity Troponin T level from equal to or above 5 to equal to or below 400 ng/L. In one embodiment, the patient has a High-sensitivity Troponin T level from equal to or above 5 to equal to or below 300 ng/L. In one embodiment, the patient has a High-sensitivity Troponin T level from equal to or above 5 to equal to or below 200 ng/L. In one embodiment, the patient has a High-sensitivity Troponin T level from equal to or above 5 to equal to or below 100 ng/L. In one embodiment, the patient has a High-sensitivity Troponin T level from equal to or above 5 to equal to or below 50 ng/L. In one embodiment, the patient has a High-sensitivity Troponin T level from equal to or above 5 to equal to or below 40 ng/L. In one embodiment, the patient has a High-sensitivity Troponin T level from equal to or above 5 to equal to or below 30 ng/L. In one embodiment, the patient has a High-sensitivity Troponin T level from equal to or above 5 to equal to or below 20 ng/L. In one embodiment, the patient has a High-sensitivity Troponin T level from equal to or above 5 to equal to or below 10 ng/L.

In one embodiment, the patient has a High-sensitivity Troponin T level from equal to or above 100 to equal to or below 95000 ng/L. In one embodiment, the patient has a High-sensitivity Troponin T level from equal to or above 100 to equal to or below 9500 ng/L. In one embodiment, the patient has a High-sensitivity Troponin T level from equal to or above 100 to equal to or below 8000 ng/L. In one embodiment, the patient has a High-sensitivity Troponin T level from equal to or above 100 to equal to or below 7000 ng/L. In one embodiment, the patient has a High-sensitivity Troponin T level from equal to or above 100 to equal to or below 6000 ng/L. In one embodiment, the patient has a High-sensitivity Troponin T level from equal to or above 100 to equal to or below 5000 ng/L. In one embodiment, the patient has a High-sensitivity Troponin T level from equal to or above 100 to equal to or below 4000 ng/L. In one embodiment, the patient has a High-sensitivity Troponin T level from equal to or above 100 to equal to or below 3000 ng/L. In one embodiment, the patient has a High-sensitivity Troponin T level from equal to or above 100 to equal to or below 2000 ng/L. In one embodiment, the patient has a High-sensitivity Troponin T level from equal to or above 100 to equal to or below 1000 ng/L. In one embodiment, the patient has a High-sensitivity Troponin T level from equal to or above 100 to equal to or below 900 ng/L. In one embodiment, the patient has a High-sensitivity Troponin T level from equal to or above 100 to equal to or below 800 ng/L. In one embodiment, the patient has a High-sensitivity Troponin T level from equal to or above 100 to equal to or below 700 ng/L. In one embodiment, the patient has a High-sensitivity Troponin T level from equal to or above 100 to equal to or below 600 ng/L. In one embodiment, the patient has a High-sensitivity Troponin T level from equal to or above 100 to equal to or below 500 ng/L. In one embodiment, the patient has a High-sensitivity Troponin T level from equal to or above 100 to equal to or below 400 ng/L. In one embodiment, the patient has a High-sensitivity Troponin T level from equal to or above 100 to equal to or below 300 ng/L. In one embodiment, the patient has a High-sensitivity Troponin T level from equal to or above 100 to equal to or below 200 ng/L.

In one embodiment, the patient has a High-sensitivity Troponin T level from equal to or above 1000 to equal to or below 95000 ng/L. In one embodiment, the patient has a High-sensitivity Troponin T level from equal to or above 1000 to equal to or below 9500 ng/L In one embodiment, the patient has a High-sensitivity Troponin T level from equal to or above 1000 to equal to or below 8000 ng/L. In one embodiment, the patient has a High-sensitivity Troponin T level from equal to or above 1000 to equal to or below 7000 ng/L. In one embodiment, the patient has a High-sensitivity Troponin T level from equal to or above 1000 to equal to or below 6000 ng/L. In one embodiment, the patient has a High-sensitivity Troponin T level from equal to or above 1000 to equal to or below 5000 ng/L. In one embodiment, the patient has a High-sensitivity Troponin T level from equal to or above 1000 to equal to or below 4000 ng/L. In one embodiment, the patient has a High-sensitivity Troponin T level from equal to or above 1000 to equal to or below 3000 ng/L. In one embodiment, the patient has a High-sensitivity Troponin T level from equal to or above 1000 to equal to or below 2000 ng/L.

In one embodiment, the patient has a High-sensitivity Troponin T level from equal to or above 6 to equal to or below 30 ng/L. In one embodiment, the patient has a High-sensitivity Troponin T level from equal to or above 6.5 to equal to or below 30 ng/L.

In one embodiment, the patient has a High-sensitivity Troponin T level selected from of equal to or above 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 20, 300, 400, 500, 600, 700, 900, 1000, 1500, 2000, 2500, 3000, 3500, 4000, 4500, 5000, 5500, 6000, 6500, 7000, 7500, 8000, 8500, 9000 and 9500 ng/L. In one embodiment, the patient has a High-sensitivity Troponin T level selected from of equal to or above 5, 6, 7, 8, 9, 10, 11, 12, 13, 14, 15, 16, 17, 18, 19, 20, 21, 22, 23, 24, 25, 26, 27, 28, 29, and 30 ng/L

In one embodiment, the patient has a High-sensitivity Troponin T level selected from the group consisting of 6.5 ng/L, 17.7 ng/L, 26.1 ng/L, 28.2 ng/L, and 7610.0 ng/L

The N-terminal prohormone of brain natriuretic peptide (NT-proBNP) is a prohormone with a 76 amino acid N-terminal inactive protein that is cleaved from the molecule to release brain natriuretic peptide 32 (BNP, also known as B-type natriuretic peptide). Both BNP and NT-proBNP levels in the blood are used for screening, diagnosis and prognosis of CHF in both acute and non-acute settings. Elevated plasma concentrations of NT-proBNP correlate with the onset of heart failure hospitalization and cardiovascular death (Mueller et al. “Heart Failure association of the European Society of cardiology practical guidance on the use of natriuretic peptide concentrations. European Journal of Heart Failure 2019; 21, 715-731 doi:10.1002/ejhf.1494).

NT-proBNP is expressed in pg/mL.

In one embodiment, the patient has a N-terminal prohormone of brain natriuretic peptide (NT-proBNP) level of equal to or above 300 pg/mL.

In one embodiment, the patient has a NT-proBNP level selected from of equal to or above 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 400, 4100, 4200, 4300, 4400 or 4500 pg/mL.

In one embodiment, the patient has a NT-proBNP level selected from of equal to or below 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 400, 4100, 4200, 4300, 4400 or 4500 pg/mL.

In one embodiment, the patient has a NT-proBNP level of equal to or above 300 to equal to or below 4300 pg/mL. In one embodiment, the patient has a NT-proBNP level of equal to or above 300 to equal to or below 2200 pg/mL. In one embodiment, the patient has a NT-proBNP level of equal to or above 300 to equal to or below 2000 pg/mL. In one embodiment, the patient has a NT-proBNP level of equal to or above 300 to equal to or below 1900 pg/mL. In one embodiment, the patient has a NT-proBNP level of equal to or above 300 to equal to or below 1600 pg/mL. In one embodiment, the patient has a NT-proBNP level of equal to or above 500 to equal to or below 3000 pg/mL.

In one embodiment, the patient has a Brain natriuretic Peptide (BNP) of equal to or above 100 pg/mL. In one embodiment, the patient has a Brain natriuretic Peptide (BNP) of equal to or above 300 pg/mL.

Blood pressure (BP) is the pressure of circulating blood against the walls of blood vessels. Most of this pressure results from the heart pumping blood through the circulatory system. When used without qualification, the term “blood pressure” refers to the pressure in a brachial artery, where it is most commonly measured. Blood pressure is usually expressed in terms of the systolic pressure (maximum pressure during one heartbeat) over diastolic pressure (minimum pressure between two heartbeats) in the cardiac cycle. It is expressed in millimeters of mercury (mmHg) above the surrounding atmospheric pressure, or in kilopascals (kPa). The difference between the systolic and diastolic pressures is known as pulse pressure, while the average pressure during a cardiac cycle is known as mean arterial pressure.

In one embodiment, the patient has a systolic blood pressure selected from equal to or above 40, 45, 50, 55, 60, 65, 70, 75, 80, 85, 90, 95, 100, 105, 110, 114, 115, 120, 125, 129, 130, 135, 140, 144, 145, 150, 155, 160, 165, 170, 175, 180, 185, and 190 mmHg. In one embodiment, the patient has a systolic blood pressure selected from equal to or below 40, 45, 50, 55, 60, 65, 70, 75, 80, 85, 90, 95, 100, 105, 110, 114, 115, 120, 125, 129, 130, 135, 140, 144, 145, 150, 155, 160, 165, 170, 175, 180, 185, and 190 mmHg.

In one embodiment, the patient has a systolic blood pressure of equal to or above 40 mmHg. In one embodiment, the patient has a systolic blood pressure of equal to or above 45 mmHg. In one embodiment, the patient has a systolic blood pressure of equal to or above 50 mmHg. In one embodiment, the patient has a systolic blood pressure of equal to or above 55 mmHg. In one embodiment, the patient has a systolic blood pressure of equal to or above 60 mmHg. In one embodiment, the patient has a systolic blood pressure of equal to or above 65 mmHg. In one embodiment, the patient has a systolic blood pressure of equal to or above 70 mmHg. In one embodiment, the patient has a systolic blood pressure of equal to or above 75 mmHg. In one embodiment, the patient has a systolic blood pressure of equal to or above 80 mmHg. In one embodiment, the patient has a systolic blood pressure of equal to or above 85 mmHg. In one embodiment, the patient has a systolic blood pressure of equal to or above 90 mmHg. In one embodiment, the patient has a systolic blood pressure of equal to or above 100 mmHg. In one embodiment, the patient has a systolic blood pressure of equal to or above 105 mmHg. In one embodiment, the patient has a systolic blood pressure of equal to or above 110 mmHg. In one embodiment, the patient has a systolic blood pressure of equal to or above 114 mmHg. In one embodiment, the patient has a systolic blood pressure of equal to or above 115 mmHg. In one embodiment, the patient has a systolic blood pressure of equal to or above 120 mmHg. In one embodiment, the patient has a systolic blood pressure of equal to or above 125 mmHg. In one embodiment, the patient has a systolic blood pressure of equal to or above 129 mmHg. In one embodiment, the patient has a systolic blood pressure of equal to or above 130 mmHg. In one embodiment, the patient has a systolic blood pressure of equal to or above 135 mmHg. In one embodiment, the patient has a systolic blood pressure of equal to or above 140 mmHg. In one embodiment, the patient has a systolic blood pressure of equal to or above 144 mmHg. In one embodiment, the patient has a systolic blood pressure of equal to or above 145 mmHg. In one embodiment, the patient has a systolic blood pressure of equal to or above 150 mmHg. In one embodiment, the patient has a systolic blood pressure of equal to or above 155 mmHg. In one embodiment, the patient has a systolic blood pressure of equal to or above 160 mmHg. In one embodiment, the patient has a systolic blood pressure of equal to or above 165 mmHg. In one embodiment, the patient has a systolic blood pressure of equal to or above 170 mmHg. In one embodiment, the patient has a systolic blood pressure of equal to or above 175 mmHg. In one embodiment, the patient has a systolic blood pressure of equal to or above 180 mmHg. In one embodiment, the patient has a systolic blood pressure of equal to or above 185 mmHg. In one embodiment, the patient has a systolic blood pressure of equal to or above 190 mmHg.

In one embodiment, the patient has a systolic blood pressure of equal to or below 40 mmHg. In one embodiment, the patient has a systolic blood pressure of equal to or below 45 mmHg. In one embodiment, the patient has a systolic blood pressure of equal to or below 50 mmHg. In one embodiment, the patient has a systolic blood pressure of equal to or below 55 mmHg. In one embodiment, the patient has a systolic blood pressure of equal to or below 60 mmHg. In one embodiment, the patient has a systolic blood pressure of equal to or below 65 mmHg. In one embodiment, the patient has a systolic blood pressure of equal to or below 70 mmHg. In one embodiment, the patient has a systolic blood pressure of equal to or below 75 mmHg. In one embodiment, the patient has a systolic blood pressure of equal to or below 80 mmHg. In one embodiment, the patient has a systolic blood pressure of equal to or below 85 mmHg. In one embodiment, the patient has a systolic blood pressure of equal to or below 90 mmHg. In one embodiment, the patient has a systolic blood pressure of equal to or below 100 mmHg. In one embodiment, the patient has a systolic blood pressure of equal to or below 105 mmHg. In one embodiment, the patient has a systolic blood pressure of equal to or below 110 mmHg. In one embodiment, the patient has a systolic blood pressure of equal to or below 114 mmHg. In one embodiment, the patient has a systolic blood pressure of equal to or below 115 mmHg. In one embodiment, the patient has a systolic blood pressure of equal to or below 120 mmHg. In one embodiment, the patient has a systolic blood pressure of equal to or below 125 mmHg. In one embodiment, the patient has a systolic blood pressure of equal to or below 129 mmHg. In one embodiment, the patient has a systolic blood pressure of equal to or below 130 mmHg. In one embodiment, the patient has a systolic blood pressure of equal to or below 135 mmHg. In one embodiment, the patient has a systolic blood pressure of equal to or below 140 mmHg. In one embodiment, the patient has a systolic blood pressure of equal to or below 144 mmHg. In one embodiment, the patient has a systolic blood pressure of equal to or below 145 mmHg. In one embodiment, the patient has a systolic blood pressure of equal to or below 150 mmHg. In one embodiment, the patient has a systolic blood pressure of equal to or below 155 mmHg. In one embodiment, the patient has a systolic blood pressure of equal to or below 160 mmHg. In one embodiment, the patient has a systolic blood pressure of equal to or below 165 mmHg. In one embodiment, the patient has a systolic blood pressure of equal to or below 170 mmHg. In one embodiment, the patient has a systolic blood pressure of equal to or below 175 mmHg. In one embodiment, the patient has a systolic blood pressure of equal to or below 180 mmHg. In one embodiment, the patient has a systolic blood pressure of equal to or below 185 mmHg. In one embodiment, the patient has a systolic blood pressure of equal to or below 190 mmHg.

In one embodiment, the patient has a systolic blood pressure of 129±15 mmHg. In one embodiment, the patient has a systolic blood pressure of equal to or above 114 to equal to or below 144 mmHg. In one embodiment, the patient has a systolic blood pressure of equal to or above 114 to equal to or below 129 mmHg. In one embodiment, the patient has a systolic blood pressure of equal to or above 129 to equal to or below 144 mmHg. In one embodiment, the patient has a systolic blood pressure of equal to or above 110 to equal to or below 114 mmHg. In one embodiment, the patient has a systolic blood pressure of equal to or above 100 to equal to or below 110 mmHg. In one embodiment, the patient has a systolic blood pressure of equal to or above 90 to equal to or below 100 mmHg.

As such, finerenone presents a new opportunity to modify risk of adverse events in patients with HF, including among those with cardio-kidney-metabolic multimorbidity. The patient to be treated may be multimorbid, i.e. the patient may have one or more other diseases. The patient may have a medical history or various medical histories. The medical terms used herein are known to those skilled in the art and are used as common in the art.

In one embodiment, the patient has a history of prior MRA use. In one embodiment, the patient has a history of atrial flutter. In one embodiment, the patient has a history of atrial fibrillation. In one embodiment, the patient has a history of coronary artery disease (CAD). In one embodiment, the patient has a history of stroke. In one embodiment, the patient has a history of transient ischemic attack (TIA). In one embodiment, the patient has a history of chronic obstructive pulmonary disease (COPD). In one embodiment, the patient has a history of metabolic syndrome. In one embodiment, the patient has a history of frailty. In one embodiment, the patient has a history of peripheral artery disease (PAD). In one embodiment, the patient has a history of obstructive sleep apnea (OSA). In one embodiment, the patient has a history of cancer. In one embodiment, the patient has a history of chronic obstructive pulmonary disease (COPD). In one embodiment, the patient has a history of resistant hypertension. In one embodiment, the patient has a history of diabetes. In one embodiment, the patient has a history of atrial flutter. In one embodiment, the patient has a history of atrial fibrillation. In one embodiment, the patient has a history of hypertension. In one embodiment, the patient has a history of myocardial infarction. In one embodiment, the patient has a history of stroke. In one embodiment, the patient has a history of LVEF equal to or below 40%. In one embodiment, the patient was hospitalized prior to treatment or prevention. In one embodiment, the patient was hospitalized for hearth failure (HHF). In one embodiment, the patient has atherosclerotic cardiovascular disease (ASCVD). In one embodiment, the patient has a history of diabetes. In one embodiment, the patient has type-2 diabetes (T2D). In one embodiment, the patient has chronic kidney disease. In one embodiment, the patient has a history of chronic kidney disease (CKD). In one embodiment, the patient has chronic kidney disease (CKD) with an eGFR equal to or above 60 mL/min/1.73 m². In one embodiment, the patient a combination of atherosclerotic cardiovascular disease (ASCVD), type 2 diabetes (T2D), and/or chronic kidney disease (CKD; defined as eGFR <60 mL/min/1.73 m²). In one embodiment, the patient has sinus rhythm or normal heart rhythm. In one embodiment, the patient has a history of structural heart disease. The structural heart disease can be indicated by increased left atrial size or left ventricular hypertrophy.

Heart rate (or pulse rate) is the frequency of the heartbeat measured by the number of contractions of the heart per minute (beats per minute, beats/min or bpm). The American Heart Association states the normal resting adult human heart rate is 60-100 bpm. Tachycardia is a high heart rate, defined as above 100 bpm at rest. Bradycardia is a low heart rate, defined as below 60 bpm at rest. When the heart is not beating in a regular pattern, this is referred to as an arrhythmia. Abnormalities of heart rate sometimes indicate disease.

In one embodiment, the patient has an average heart rate (resting) selected from of equal to or above 40, 45, 50, 55, 59, 60, 65, 70, 71, 75, 80, 83, 85, 90, 95, 100, 105, 110, 115, and 120 beats/min.

The average heart rate is a resting heart rate.

In one embodiment, the patient has an average (resting) heart rate of 71+12 beats/min. In one embodiment, the patient has an average (resting) heart rate of equal to or above 45 to equal to or below 59 beats/min. In one embodiment, the patient has an average (resting) heart rate of equal to or above 59 to equal to or below 83 beats/min. In one embodiment, the patient has an average (resting) heart rate of equal to or above 59 to equal to or below 71 beats/min. In one embodiment, the patient has an average (resting) heart rate of equal to or above 71 to equal to or below 83 beats/min. In one embodiment, the patient has an average (resting) heart rate of equal to or above 83 beats/min to equal to or below 110 beats/min. In one embodiment, the patient has an average (resting) heart rate of equal to or above 90 beats/min to equal to or below 110 beats/min. In one embodiment, the patient has an average (resting) heart rate of equal to or above 90 beats/min to equal to or below 100 beats/min.

The patient can receive further medication or comedication. This can be, e.g., the case if the patients is multimorbid.

In one embodiment, the patient further receives any one of the medicaments selected from potassium-sparing diuretic, osmotic diuretic, carbonic anhydrase inhibitor, digoxin, nitrates, loop diuretics, thiazide diuretics, beta-blocker, ACE inhibitor (ACEi), angiotensin-receptor-blocker (ARB), Angiotensin Receptor-Neprilysin Inhibitor (ARNI), Calcium Channel Blockers, Sodium-glucose Cotransporter-2 Inhibitor (SGLT-2i), Sodium-glucose Cotransporter-1 Inhibitor (SGLT-li), potassium supplements, potassium lowering agents, potassium binders, centrally acting antihypertensives, cytochrome P450 isoenzyme 3A4 (CYP3A4) inhibitors, CYP3A4 inducers, aspirin, statins, organic anion transporting polypeptides (OATP) substrates, anti-diabetic drugs, insulin, insulin analogues, dipeptidyl peptidase 4 inhibitors, glucagon-like peptide-1 (GLP-1) agonists, biguanides, sulfonylureas, alpha glucosidase inhibitors, metiglinides, thiazolidinediones, CYP3A4 inducer, CYP3A4 inhibitor and mixtures thereof.

In one embodiment, the patient further receives a Sodium-glucose Cotransporter-2 Inhibitor (SGLT-2i) and a glucagon-like peptide-1 (GLP-1) agonist. In one embodiment thereof, the SGLT-2i is selected from is selected from the group consisting of canagliflozin, dapagliflozin, empagliflozin, ertigliflozin, ipragliflozin, luseogliflozin, remogliflozin, tofogliflozin, the pharmaceutical acceptable salts, solvates, hydrates, and polymorphs thereof, and combinations thereof. In one embodiment thereof, the SGLT-2i is dapagliflozin. In one embodiment thereof, the SGLT-2i is empagliflozin. In one embodiment thereof, the SGLT-2i is canagliflozin.

In one embodiment thereof, the GLP-1 is selected from dulaglutide, exenatide, liraglutide, lixisenatine, semaglutine, tirzepatide, the pharmaceutical acceptable salts, solvates, hydrates, and polymorphs thereof and combinations thereof. In one embodiment thereof, the SGLT-2i is empagliflozin and the GLP-1 is selected from dulaglutide, exenatide, liraglutide, lixisenatine, semaglutine, tirzepatide, the pharmaceutical acceptable salts, solvates, hydrates, and polymorphs thereof, and combinations thereof. In one embodiment thereof, the SGLT-2i is canagliflozin and the GLP-1 is selected from dulaglutide, exenatide, liraglutide, lixisenatine, semaglutine, tirzepatide, the pharmaceutical acceptable salts thereof and combinations thereof. In one embodiment thereof, the SGLT-2i is Dapagliflozin and the GLP-1 is selected from dulaglutide, exenatide, liraglutide, lixisenatine, semaglutine, tirzepatide, the pharmaceutical acceptable salts thereof and combinations thereof.

In one embodiment, the patient further receives a potassium-sparing diuretic. In one embodiment, the patient further receives an osmotic diuretic. In one embodiment, the patient further receives a carbonic anhydrase inhibitor. In one embodiment, the patient further receives digoxin. In one embodiment, the patient further receives a nitrate. In one embodiment, the patient further receives a loop diuretic. In one embodiment, the patient further receives a thiazide diuretic. In one embodiment, the patient further receives a beta-blocker. In one embodiment, the patient further receives an ACE inhibitor (ACEi). In one embodiment, the patient further receives an angiotensin-receptor-blocker (ARB). In one embodiment, the patient further receives an Angiotensin Receptor-Neprilysin Inhibitor (ARNI). In one embodiment, the patient further receives a Calcium Channel Blocker. In one embodiment, the patient further receives a Sodium-glucose Cotransporter-2 Inhibitor (SGLT-2i). In one embodiment, the patient further receives a Sodium-glucose Cotransporter-1 Inhibitor (SGLT-li). In one embodiment, the patient further receives a potassium supplement. In one embodiment, the patient further receives a potassium lowering agent. In one embodiment, the patient further receives a potassium binder. In one embodiment, the patient further receives an antihypertensive agent. In one embodiment, the patient further receives a centrally acting antihypertensive. In one embodiment, the patient further receives a cytochrome P450 isoenzyme 3A4 (CYP3A4) inhibitor. In one embodiment, the patient further receives a CYP3A4 inducer. In one embodiment, the patient further receives aspirin. In one embodiment, the patient further receives a statin. In one embodiment, the patient further receives an organic anion transporting polypeptides (OATP) substrate. In one embodiment, the patient further receives an anti-diabetic drug. In one embodiment, the patient further receives insulin. In one embodiment, the patient further receives an insulin analogue. In one embodiment, the patient further receives a dipeptidyl peptidase 4 inhibitor. In one embodiment, the patient further receives a glucagon-like peptide-1 agonists. In one embodiment, the patient further receives a biguanide. In one embodiment, the patient further receives a sulfonylurea. In one embodiment, the patient further receives an alpha glucosidase inhibitor. In one embodiment, the patient further receives a metiglinide. In one embodiment, the patient further receives a thiazolidinedione.

In one embodiment, the patient further receives an alpha blocker. In one embodiment, the patient further receives a beta blocker. In one embodiment the alpha blocker and/or beta blocker is selected from acebutolol, amlodipine, bisoprolol, arotinolol, atenolol, betaxolol, bimatoprost, timolol, bisoprolol, perindopril, bromonidine, brinzolamide, carteolol, lantanoprost, carvediol, ivabradine, dorzolamide, esmolol, hydrochlorothiazide, metroprolol, nebivolol, labetalol, landiolol, nadolol, nebiviolol, pilocarpine, propranolol, sotalol, tafluprost, timolol, travoprost, the pharmaceutical acceptable salts thereof and combinations thereof.

In one embodiment, the patient further receives an angiotensin-converting enzyme (ACE) inhibitor. In one embodiment the ACE inhibitor is selected from ramipril, alacepril, amlodipine, perindopril, benazepril, captopril, enalapril, fosinopril, lisinopril, moexipril, perindopril, ramipril, zofenopril, clizapril, imidapril, quinapril, trandolapril, the pharmaceutical acceptable salts thereof and combinations thereof.

In one embodiment, the patient further receives an angiotensin receptor blockers (ARBs) selected from allisartan, azilsartan, candesartan, olmesartan, valsartan, irbesartan, losartan, telmisartan, candesartan, eprosartan, fimasartan, the pharmaceutical acceptable salts thereof and combinations thereof.

In one embodiment, the patient further receives an angiotensin receptor neprilysin inhibitors (ARNIs). In one embodiment the ARNI is sacubitril or the pharmaceutical acceptable salts thereof. In one embodiment the patient receives sacubitril, valsartan or the pharmaceutical acceptable salts thereof.

In one embodiment, the patient further receives atorvastatin the pharmaceutical acceptable salts thereof. In one embodiment the patient receives a further medication selected from acetylsalicylic acid, valsartan, clopidogrel, ramipril, perindopril, ezetimibe, the pharmaceutical acceptable salts thereof and combinations thereof.

In one embodiment, the patient further receives a biguanide. In one embodiment the patient receives a medication selected from alogliptin, metformin, canagliflozin, dapagliflozin, empagliflozin, ertugliflozin, gemigliptin, glibenclamide, gliciclazide, linagliptide, pioglitazone, sitagliptin, teneligliptin, vildagliptin, vogliibose, pioglitazin, the pharmaceutical acceptable salts thereof and combinations thereof.

In one embodiment, the patient further receives a calcium channel blocker. In one embodiment, the patient further receives a calcium channel blocker selected from amlodipine, azelnidipine, barnidipine, benidipine, bepridil, cilnidipine, diltiazem, efonidipine, lacidinipine, lercanidipine, levamlodipine, manidipine, nicardipine, nifedipine, nilvadepine, nimodipine, nisoldipine, nitrendipine, perhexiline, verapamil, the pharmaceutical acceptable salts thereof and combinations thereof. In one embodiment, the patient further receives a medication selected from amlodipine, atorvastatin, candesartan, hydrochlorotiazide (HCT), perindopril, azilsartan, benazepril, ramipril, olmesartan, valsartan, indapipamide, perindopril, irbesartan, lisinopril, lorsartan, azelnidipine, barnidipine, benidipine, bepridil, cilnidipine, diltiazem, efonidipine, lacidinipine, lercanidipine, levamlodipine, manidipine, nicardipine, nifedipine, nilvadepine, nimodipine, nisoldipine, nitrendipine, perhexiline, verapamil, perindopril, ramipril, rosuvastatin, telmisartan, atorvastatin, benazepril, the pharmaceutical acceptable salts thereof and combinations thereof.

In one embodiment, the patient further receives a dipeptidyl peptidase-4 (DDP4) inhibitor. In one embodiment, the DDP4 inhibitor is selected from alogliptin, anagliptin, tenegliptin, saxagliptin, linagliptin, gemigliptin, gosogliptin, sitagliptin, tenegliptin, vildagliptin, the pharmaceutical acceptable salts thereof and combinations thereof. In one embodiment, the patient receives a medication selected from alogliptin, anagliptin, tenegliptin, saxagliptin, linagliptin, gemigliptin, gosogliptin, sitagliptin, tenegliptin, vildagliptin, metformin, pioglitazone, cangliflozin, dapagliflozin, empagliflozin, ipragliflozin, the pharmaceutical acceptable salts thereof and combinations thereof.

In one embodiment, the patient further receives a diuretic. In one embodiment, the diuretic is selected from tropolone, acetazolamide, altizide, spironolactone, amiloride, hydrochlorothiazide, clopamide, chlorthalidone, indapamide, furosemide, azosemide, glycerol, cendroflumethiazide, brimonidine, bumetanide, butizide, chlorothiazide, isosorbide, triamterene, dorzolamide, metolazone, protheobromide, torasemide, tolvaptan, trochloromethiazid, xipamide, theobromide, hydrocortisone, pharmaceutical acceptable salts thereof and combinations thereof. In one embodiment, the patient further receives a medicationelected from tropolone, acetazolamide, altizide, spironolactone, amiloride, hydrochlorothiazide, chlorthalidone, indapamide, furosemide, azosemide, glycerol, cendroflumethiazide, brimonidine, bumetanide, butizide, chlorothiazide, isosorbide, triamterene, dorzolamide, metolazone, protheobromide, torasemide, tolvaptan, trichloromethiazide, xipamide, theobromide, hydrocortisone, tolvaptan, torasemide, trichloromethiazide, urea, xipamide, amlodipine, olmesatran, perindopril, atenolol, azosemide, benazepril, potassium chloride, bisoprolol, timolol, candesartan, captopril, cetomacrogel, cilapril, clobetasol, clopamide, clotrimazole, timolol, enalapril, etacrynic acid, irbesartan, losartan, telmisartan, nebivolol, zofenopril, rosuvastatin, the pharmaceutical acceptable salts thereof and combinations thereof.

In one embodiment, the patient further receives a statin. In one embodiment, the statin is selected from fluvastatin, atorvastatin, lovastatin, pitavastatin, pravastatin, rosuvastatin, simvastatin, astaxanthin, the pharmaceutical acceptable salts thereof and combinations thereof.

In one embodiment, the patient further receives a glucagon-like peptide 1 (GLP-1) agonist. In one embodiment, the GLP-1 agonist is selected from dulaglutide, exenatide, liraglutide, lixisenatide, semaglutide, tirzepatide, the pharmaceutical acceptable salts thereof and combinations thereof.

In one embodiment, the patient further receives a sodium-glucose cotransporter-2 (SGLT2) inhibitor. In one embodiment the SGLT2 inhibitor is selected from canagliflozin, dapagliflozin, empagliflozin, ertigliflozin, ipragliflozin, luseogliflozin, remogliflozin, tofogliflozin, the pharmaceutical acceptable salts thereof and combinations thereof.

In one embodiment, the patient further receives a sufonamide. In one embodiment the sulfonamide is selected from glibenclamide, gliclazide, glimepiride, glipizide, gliquidone, tolbutamide, the pharmaceutical acceptable salts thereof and combinations thereof.

In one embodiment, the patient further receives a potassium binder. In one embodiment, the potassium binder is selected from potassium polystytrene, calcium polystyrene sulfonate, patiromer calcium, patiromer, sorbitex calcium, sodium polystyrene sulfonate, sodium zirconium cyclosilicate, the pharmaceutical acceptable salts thereof and combinations thereof.

In one embodiment, the patient further receives a mineralocorticoid receptor antagonists (MRAs). In one embodiment the MRA is selected from spironolactone, canrenone, eplerenone, potassium canrenoate, the pharmaceutical acceptable salts thereof and combinations thereof.

In one embodiment, the patient further receives ivabradine or the pharmaceutical acceptable salts thereof.

In one embodiment, the patient further receives an insulin. In one embodiment, the insulin is selected from a natural, synthetic, modified, porcine, bovine, insulin derivative, insulin analogoue, long acting, short acting, a pharmaceutical acceptable salt thereof and combinations thereof. In one embodiment, the insulin is selected from insuline aspart, aspart protamine, protamine, dediglec, bovine, porcine, glargine, glargine biosimiliar, glusine, human, lispro, lispro biosimilar, lispro protamine, human, zinc suspension, analogues, the pharmaceutical acceptable salts thereof and combinations thereof.

In one embodiment, the patient further receives an oral antidiabetic. In one embodiment, the oral antidiabetic is selected from alpha-glucosidase inhibitors, amylin analogs, dipeptidyl peptidase 4 inhibitors, incretin mimetics, insulin, meglitinides, non-sulfonylureas, SGLT-2, sulfonylureas, and thiazolidinediones, the pharmaceutical acceptable salts thereof and combinations thereof. In one embodiment, the oral antidiabetic is selected from acarbose, miglitol, pramlintide, alogliptan, linagliptan, saxagliptin, sitagliptin, albiglutide, dulaglutide, exenatide, liraglutide, lixisenatide, insulins, nateglinide, repaglinide, metformin, canagliflozin, dapagliflozin, empagliflozin, chlorpropamide, glimepiride, glipizide, glyburide, tolazamide, tolbutamide, rosiglitazone, pioglitazone, the pharmaceutical acceptable salts thereof and combinations thereof. In one embodiment, the further medication is selected from (RS)-3-METHYL-2-OXOVALERIANIC ACID CALCIUM; (RS)-3-METHYL-2-OXOBUTYRIC ACID CALCIUM; CALCIUM (RS)-4-METHYL-2-OXOVALERIANAT; CALCIUM 2-OXO-3-PHENYLPROPIONATE, DESMENINOL CALCIUM, HISTIDINE, LYSINE ACETATE, THREONINE, TRYPTOPHAN, TYROSINE, ACARBOSE, ACETYLCYSTEINE, ALOGLIPTIN, ALOGLIPTIN BENZOATE, ALOGLIPTIN BENZOATE, METFORMIN HYDROCHLORIDE, ALOGLIPTIN BENZOATE, PIOGLITAZONE HYDROCHLORIDE, ANAGLIPTIN, ATORVASTATIN, ATORVASTATIN CALCIUM, BIGUANIDE, CALCIUM CARBONATE, MAGNESIUM CARBONATE, POTASSIUM CARBONATE; SODIUM BICARBONATE, SODIUM PHOSPHATE, CALCIUM DOBESILATE, CANAGLIFLOZIN, CANAGLIFLOZIN HEMIHYDRATE, CANAGLIFLOZIN HEMIHYDRATE, METFORMIN, CANAGLIFLOZIN HEMIHYDRATE, TENELIGLIPTIN HYDROBROMIDE, CHLORPHENAMINE MALEATE, PHENYLEPHRINE HYDROCHLORIDE, PHENYLPROPANOLAMINE HYDROCHLORIDE; PHENYLTOLOXAMINE CITRATE, CYSTEINE, GLYCINE, GLYCYRRHIZIC ACID, AMMONIUM SALT, DAPAGLIFLOZIN, SAXAGLIPTIN, DEXAMETHASONE, DIGOXIN, DULAGLUTIDE, EMPAGLIFLOZIN, ENOXAPARIN, ERTUGLIFLOZIN, EXENATIDE, GEMIGLIPTIN TARTRATE, GLIBENCLAMIDE, GLICLAZIDE, GLIMEPIRIDE, GLIPIZIDE, GLIQUIDONE, GLUCAGON, GLUCOSE, GOSOGLIPTIN, HYDROCHLOROTHIAZIDE, IMEGLIMIN, INSULIN ASPART, INSULIN ASPART PROTAMINE (CRYSTALLINE), INSULIN DEGLUDEC, INSULIN GLARGINE, INSULIN HUMAN, INSULIN HUMAN INJECTION, ISOPHANE, INSULIN HUMAN, INSULIN LISPRO PROTAMINE SUSPENSION, INSULIN LISPRO, IPRAGLIFLOZIN L-PROLINE, IPRAGLIFLOZIN L-PROLINE; SITAGLIPTIN PHOSPHATE MONOHYDRATE, KALLIDINOGENASE, LEVOTHYROXINE SODIUM, LINAGLIPTIN, LIRAGLUTIDE, LIXISENATIDE, LUSEOGLIFLOZIN, MACROGOL, MECOBALAMIN, MELILOTUS OFFICINALIS EXTRACT, METFORMIN, MIGLITOL, MITIGLINIDE, NATEGLINIDE, PIOGLITAZONE, PITAVASTATIN, QUETIAPINE FUMARATE, REMOGLIFLOZIN ETABONATE, REPAGLINIDE, ROSUVASTATIN, SAXAGLIPTIN, SEMAGLUTIDE, SITAGLIPTIN, SULTAMICILLIN, TELMISARTAN, THIOCTIC ACID, TIRZEPATIDE, TOFOGLIFLOZIN, TOLBUTAMIDE, VILDAGLIPTIN, VOGLIBOSE, the pharmaceutical acceptable salts thereof and combinations thereof.

In one embodiment, the patient further receives a CYP3A4 inducer. The patient can receive the CYP3A4 inducer prior and/or during the treatment. In one embodiment, the patient further receives a weak CYP3A4 inducer. In one embodiment, the patient further receives a moderate CYP3A4 inducer. In one embodiment, the patient further receives a strong CYP3A4 inducer. In one embodiment, the patient further receives a CYP3A4 inducer of unspecified potency. Strong and moderate CYP3A4 inducers are drugs that decrease the AUC of sensitive (index) substrates of a given pathway where CYP3A4 is involved by ≥80 percent and ≥50 to <80 percent, respectively. Weak inducers decrease the AUC by ≥20 to <50 percent.

The above definitions of strong and moderate CYP3A4 inducers come from FDA Guidance. See Clinical Drug Interaction Studies—Cytochrome P450 Enzyme- and Transporter-Mediated Drug Interactions Guidance for Industry, U.S. Department of Health and Human Services, Food and Drug Administration, Center for Drug Evaluation and Research (CDER), January 2020, (https://www.fda.gov/Drugs/GuidanceComplianceRegulatoryInformation/Guidances/default.htm) accessed Nov. 1, 2023 and incorporated herein by reference. Other sources may use a different classification system for strong and moderate inducers resulting in some agents being classified differently.

Examples, but not limited to, strong CYP3A4 inducers can be carbamazepine, antiandrogens (enzalutamide, apalutamide), primidone, phenytoin (anticonvulsant), rifampin, rifampicin, rifapentine, rifabutin, alogliptin, pioglitazone, aprepitant, carbamazepine, clobazam, eleutherocococcus senticosus, hypericum perforatum, St John's wort, enzalutamide, apalutamide, methformin, oxcarbazepine, prednisone, quercetin, avasimibe, fosphenytoin, ivosidenib, lumacaftor, and/or mitotane.

In one embodiment, the strong CYP3A4 inducer and/or a combination that comprises at least one strong CYP3A4 inducer is selected from:
CARBAMAZEPINE
ELEUTHEROCOCCUS SENTICOSUS ROOT WITH RHIZOME; HYPERICUM
PERFORATUM HERB
ENZALUTAMIDE
HYPERICUM PERFORATUM EXTRACT
PHENYTOIN
PHENYTOIN SODIUM
RIFAMPICIN
RIFAMPICIN SODIUM
   and mixtures thereof. In one embodiment thereof, the patient receives an amount of the compound according to formula (I) of 5 to 80 mg. In one embodiment thereof, the patient receives an amount of the compound according to formula (I) of 5 mg. In one embodiment thereof, the patient receives an amount of the compound according to formula (I) of 10 mg. In one embodiment thereof, the patient receives an amount of the compound according to formula (I) of 20 mg. In one embodiment thereof, the patient receives an amount of the compound according to formula (I) of 30 mg. In one embodiment thereof, the patient receives an amount of the compound according to formula (I) of 40 mg. In one embodiment thereof, the patient receives an amount of the compound according to formula (I) of 60 mg. In one embodiment thereof, the patient receives an amount of the compound according to formula (I) of 80 mg. The amount of the compound according to formula (I) in these embodiments can be adjusted based on the CYP3A4 inducer. The adjustment can be up-titration, down-titration, maintenance or interruption of the treatment with the compound according to formula (I). Multiple embodiments of dose adjustments are described in detail below. These can be applied based on the co-medication with a CYP3A4 inducer. These can also be applied based on the amount and/or type of the CYP3A4 inducer the patient receives.

The terms “up-titration” and “down-titration” can be used as synonyms for “increasing” and “decreasing,” respectively, in a method of treating comprising administering to a patient in need thereof a compound of formula (I).

The terms “up-titration” and “down-titration” can be used as synonyms for “increasing” and “decreasing,” respectively, in a method of preventing or treating heart failure of treating comprising administering to a patient in need thereof a compound of formula (I).

The terms “up-titration” and “down-titration” can be used as synonyms for “increasing” and “decreasing,” respectively, in a method of preventing or treating heart failure comprising administering to a patient in need thereof a compound of formula (I).

The terms “up-titration” and “down-titration” can be used as synonyms for “increasing” and “decreasing,” respectively, in a method of preventing or treating heart failure comprising administering to a patient in need thereof a compound of formula (I), wherein the heart failure is selected from symptomatic heart failure, heart failure with improved ejection fraction (HFimpEF), heart failure with mid-range ejection fraction (HFmrEF), heart failure preserved ejection fraction (HFpEF), heart failure with reduced ejection fraction (HFrEF), chronic heart failure (CHF), congestive heart failure, acute heart failure, chronic heart failure, worsening chronic heart failure (WCHF), hospitalization for heart failure.

In one embodiment, the strong CYP3A4 inducer and/or a combination that comprises at least one strong CYP3A4 inducer is selected from:
CARBAMAZEPINE
PHENYTOIN SODIUM
   and mixtures thereof, wherein the patient receive an amount of the compound according to formula (I) of 10 mg.

In one embodiment, the strong CYP3A4 inducer and/or a combination that comprises at least one strong CYP3A4 inducer is selected from:
CARBAMAZEPINE
ENZALUTAMIDE
HYPERICUM PERFORATUM EXTRACT
PHENYTOIN
PHENYTOIN SODIUM
RIFAMPICIN
   and mixtures thereof, wherein the patient receive an amount of the compound according to formula (I) of 20 mg.

In one embodiment, the strong CYP3A4 inducer and/or a combination that comprises at least one strong CYP3A4 inducer is selected from:
CARBAMAZEPINE
PHENYTOIN
RIFAMPICIN
   and mixtures thereof, wherein the patient receive an amount of the compound according to formula (I) of 40 mg.

Non-limiting examples of moderate CYP3A4 inducers include upadacitinib, benzalkomium chloride, dexamethasone, phenylmercuric nitrate, modafinil, nafcillin, asunaprevir, beclabuvir, daclatasvir, bosentan, cenobamate, dabrafenib, efavirenz, elagolix, etravirine, lersivirine, lesinurad, lopinavir, lorlatinib, metamizole (dipyrone), mitapivat, pexidartinib, phenobarbital, primidone, repotrectinib, rifabutin, semagacestat, sotorasib, talviraline, telotristat ethyl, thioridazine.

In one embodiment, the moderate CYP3A4 inducer and/or a combination that comprises at least one moderate CYP3A4 inducer is selected from:
BENZALKONIUM CHLORIDE; DEXAMETHASONE; PHENYLMERCURIC NITRATE
BENZALKONIUM CHLORIDE; DEXAMETHASONE; RESORCINOL; TETRACAINE HYDROCHLORIDE
BENZETHONIUM CHLORIDE; CETYLPYRIDINIUM CHLORIDE; DEXAMETHASONE; HEXYLRESORCINOL
BORIC ACID; DEXAMETHASONE SODIUM PHOSPHATE; SODIUM BORATE; THIOMERSAL
CARMELLOSE; DEXAMETHASONE SODIUM METASULFOBENZOATE
CHLORAMPHENICOL; DEXAMETHASONE
CIPROFLOXACIN HYDROCHLORIDE; DEXAMETHASONE
CYANOCOBALAMIN; DEXAMETHASONE SODIUM PHOSPHATE; PYRIDOXINE HYDROCHLORIDE; THIAMINE HYDROCHLORIDE
DEXAMETHASONE
DEXAMETHASONE ACETATE
DEXAMETHASONE DIPROPIONATE
DEXAMETHASONE PHOSPHATE
DEXAMETHASONE PHOSPHATE; POLYMYXIN B SULFATE; TRIMETHOPRIM
DEXAMETHASONE PHOSPHATE; TOBRAMYCIN
DEXAMETHASONE SODIUM METASULFOBENZOATE
DEXAMETHASONE SODIUM PHOSPHATE
DEXAMETHASONE SODIUM PHOSPHATE; LEVOFLOXACIN HEMIHYDRATE
DEXAMETHASONE SODIUM PHOSPHATE; NEOMYCIN SULFATE
DEXAMETHASONE SODIUM PHOSPHATE; NEOMYCIN SULFATE; POLYMYXIN B SULFATE
DEXAMETHASONE SODIUM PHOSPHATE; NETILMICIN SULFATE
DEXAMETHASONE SODIUM PHOSPHATE; TOBRAMYCIN
DEXAMETHASONE SODIUM SUCCINATE
DEXAMETHASONE VALERATE
DEXAMETHASONE; FRAMYCETIN SULFATE; GRAMICIDIN
DEXAMETHASONE; GENTAMICIN
DEXAMETHASONE; MOXIFLOXACIN
DEXAMETHASONE; MOXIFLOXACIN HYDROCHLORIDE
DEXAMETHASONE; NEOMYCIN SULFATE; POLYMYXIN B SULFATE
DEXAMETHASONE; NEOMYCIN; POLYMYXIN B
DEXAMETHASONE; TOBRAMYCIN
MODAFINIL
NAFCILLIN
   and mixtures thereof. In one embodiment thereof, the patient receives an amount of the compound according to formula (I) of 5 to 80 mg. In one embodiment thereof, the patient receives an amount of the compound according to formula (I) of 5 mg. In one embodiment thereof, the patient receives an amount of the compound according to formula (I) of 10 mg. In one embodiment thereof, the patient receives an amount of the compound according to formula (I) of 20 mg. In one embodiment thereof, the patient receives an amount of the compound according to formula (I) of 30 mg. In one embodiment thereof, the patient receives an amount of the compound according to formula (I) of 40 mg. In one embodiment thereof, the patient receives an amount of the compound according to formula (I) of 60 mg. In one embodiment thereof, the patient receives an amount of the compound according to formula (I) of 80 mg. The amount of the compound according to formula (I) in these embodiments can be adjusted based on the CYP3A4 inducer. The adjustment can be up-titration, down-titration, maintenance or interruption of the treatment with the compound according to formula (I). Multiple embodiments of dose adjustments are described in detail below. These can be applied based on the co-medication with a CYP3A4 inducer. These can also be applied based on the amount and/or type of the CYP3A4 inducer the patient receives.

In one embodiment, the moderate CYP3A4 inducer and/or a combination that comprises at least one moderate CYP3A4 inducer is selected from:
BENZALKONIUM CHLORIDE; DEXAMETHASONE; RESORCINOL; TETRACAINE HYDROCHLORIDE
DEXAMETHASONE
DEXAMETHASONE ACETATE
DEXAMETHASONE DIPROPIONATE
DEXAMETHASONE PHOSPHATE
DEXAMETHASONE SODIUM PHOSPHATE
DEXAMETHASONE SODIUM PHOSPHATE; NEOMYCIN SULFATE
DEXAMETHASONE SODIUM PHOSPHATE; NETILMICIN SULFATE
DEXAMETHASONE SODIUM PHOSPHATE; TOBRAMYCIN
DEXAMETHASONE; NEOMYCIN SULFATE; POLYMYXIN B SULFATE
DEXAMETHASONE; TOBRAMYCIN
MODAFINIL
   and mixtures thereof, wherein the patient receive an amount of the compound according to formula (I) of 10 mg.

In one embodiment, the moderate CYP3A4 inducer and/or a combination that comprises at least one moderate CYP3A4 inducer is selected from:
BENZETHONIUM CHLORIDE; CETYLPYRIDINIUM
CHLORIDE; DEXAMETHASONE; HEXYLRESORCINOL
CHLORAMPHENICOL; DEXAMETHASONE
CYANOCOBALAMIN; DEXAMETHASONE SODIUM PHOSPHATE; PYRIDOXINE HYDROCHLORIDE; THIAMINE HYDROCHLORIDE
DEXAMETHASONE
DEXAMETHASONE ACETATE
DEXAMETHASONE DIPROPIONATE
DEXAMETHASONE PHOSPHATE
DEXAMETHASONE PHOSPHATE; TOBRAMYCIN
DEXAMETHASONE SODIUM PHOSPHATE
DEXAMETHASONE SODIUM PHOSPHATE; NEOMYCIN SULFATE
DEXAMETHASONE SODIUM PHOSPHATE; NEOMYCIN SULFATE; POLYMYXIN B SULFATE
DEXAMETHASONE SODIUM PHOSPHATE; NETILMICIN SULFATE
DEXAMETHASONE SODIUM SUCCINATE
DEXAMETHASONE VALERATE
DEXAMETHASONE; FRAMYCETIN SULFATE; GRAMICIDIN
DEXAMETHASONE; GENTAMICIN
DEXAMETHASONE; MOXIFLOXACIN
DEXAMETHASONE; MOXIFLOXACIN HYDROCHLORIDE
DEXAMETHASONE; NEOMYCIN SULFATE; POLYMYXIN B SULFATE
DEXAMETHASONE; NEOMYCIN; POLYMYXIN B
DEXAMETHASONE; TOBRAMYCIN
MODAFINIL
   and mixtures thereof, wherein the patient receive an amount of the compound according to formula (I) of 20 mg.

In one embodiment, the moderate CYP3A4 inducer and/or a combination that comprises at least one moderate CYP3A4 inducer is selected from:
BENZALKONIUM CHLORIDE; DEXAMETHASONE; PHENYLMERCURIC NITRATE
CARMELLOSE; DEXAMETHASONE SODIUM METASULFOBENZOATE
CIPROFLOXACIN HYDROCHLORIDE; DEXAMETHASONE
DEXAMETHASONE
DEXAMETHASONE ACETATE
DEXAMETHASONE DIPROPIONATE
DEXAMETHASONE PHOSPHATE
DEXAMETHASONE PHOSPHATE; POLYMYXIN B SULFATE; TRIMETHOPRIM
DEXAMETHASONE SODIUM METASULFOBENZOATE
DEXAMETHASONE SODIUM PHOSPHATE
DEXAMETHASONE SODIUM PHOSPHATE; NEOMYCIN SULFATE
DEXAMETHASONE VALERATE
DEXAMETHASONE; FRAMYCETIN SULFATE; GRAMICIDIN
DEXAMETHASONE; MOXIFLOXACIN HYDROCHLORIDE
DEXAMETHASONE; NEOMYCIN SULFATE; POLYMYXIN B SULFATE
DEXAMETHASONE; TOBRAMYCIN
   and mixtures thereof, wherein the patient receive an amount of the compound according to formula (I) of 40 mg.

In one embodiment, the moderate CYP3A4 inducer and/or a combination that comprises at least one moderate CYP3A4 inducer is selected from:
DEXAMETHASONE; TOBRAMYCIN
   and mixtures thereof, wherein the patient receive an amount of the compound according to formula (I) of 80 mg.

Examples, but not limited to, CYP3A4 inducers of unspecified potency can be anticonvulsants, mood stabilizers (oxcarbazepine, topiramate), barbiturates (phenobarbital, butalbital), bactericidals (rifampicin, rifabutin), non-nucleoside reverse-transcriptase inhibitors (efavirenz, nevirapine), troglitazone (hypoglycemic), glucocorticoids (blood glucose increase, immunosuppressive), capsaicin, brigatinib, clobazam, dabrafenib, elagolix, eslicarbazepine, letermovir, lorlatinib, oritavancin, perampanel, and/or telotristat.

In one embodiment, the CYP3A4 inducer of unspecified potency and/or a combination that comprises at least one CYP3A4 inducer of unspecified potency is selected from:
AMOBARBITAL; CAFFEINE; CODEINE PHOSPHATE; OCTIBENZONIUM BROMIDE; PARACETAMOL
CLOTRIMAZOLE; HEXAMIDINE ISETIONATE; PREDNISOLONE ACETATE
CYPROTERONE
GINKGO BILOBA LEAF; LIGUSTRAZINE PHOSPHATE
LIGUSTRAZINE HYDROCHLORIDE
LIGUSTRAZINE HYDROCHLORIDE; SALVIA MILTIORRHIZA
LIGUSTRAZINE HYDROCHLORIDE; SALVIA MILTIORRHIZA ROOT WITH RHIZOME
LIGUSTRAZINE PHOSPHATE
PHENYLBUTAZONE; PREDNISOLONE
PREDNISOLONE
PREDNISOLONE ACETATE
PREDNISOLONE ACETATE; SULFACETAMIDE SODIUM
PREDNISOLONE PHOSPHATE
PREDNISOLONE SODIUM PHOSPHATE
PREDNISOLONE SODIUM SUCCINATE
PREDNISOLONE STEAGLATE
PREDNISOLONE VALEROACETATE
PREDNISOLONE; TETRYZOLINE HYDROCHLORIDE
RELUGOLIX
RIFAMYCIN SODIUM
   and mixtures thereof. In one embodiment thereof, the patient receives an amount of the compound according to formula (I) of 5 to 80 mg. In one embodiment thereof, the patient receives an amount of the compound according to formula (I) of 5 mg. In one embodiment thereof, the patient receives an amount of the compound according to formula (I) of 10 mg. In one embodiment thereof, the patient receives an amount of the compound according to formula (I) of 20 mg. In one embodiment thereof, the patient receives an amount of the compound according to formula (I) of 30 mg. In one embodiment thereof, the patient receives an amount of the compound according to formula (I) of 40 mg. In one embodiment thereof, the patient receives an amount of the compound according to formula (I) of 60 mg. In one embodiment thereof, the patient receives an amount of the compound according to formula (I) of 80 mg. The amount of the compound according to formula (I) in these embodiments can be adjusted based on the CYP3A4 inducer. The adjustment can be up-titration, down-titration, maintenance or interruption of the treatment with the compound according to formula (I). Multiple embodiments of dose adjustments are described in detail below. These can be applied based on the co-medication with a CYP3A4 inducer. These can also be applied based on the amount and/or type of the CYP3A4 inducer the patient receives.

In one embodiment, the CYP3A4 inducer of unspecified potency and/or a combination that comprises at least one CYP3A4 inducer of unspecified potency is selected from:
LIGUSTRAZINE HYDROCHLORIDE; SALVIA MILTIORRHIZA
LIGUSTRAZINE HYDROCHLORIDE; SALVIA MILTIORRHIZA ROOT WITH RHIZOME
PREDNISOLONE
PREDNISOLONE ACETATE
PREDNISOLONE SODIUM PHOSPHATE
PREDNISOLONE SODIUM SUCCINATE
PREDNISOLONE VALEROACETATE
PREDNISOLONE; TETRYZOLINE HYDROCHLORIDE
   and mixtures thereof, wherein the patient receive an amount of the compound according to formula (I) of 10 mg.

In one embodiment, the CYP3A4 inducer of unspecified potency and/or a combination that comprises at least one CYP3A4 inducer of unspecified potency is selected from:
CYPROTERONE
PHENYLBUTAZONE; PREDNISOLONE
PREDNISOLONE
PREDNISOLONE ACETATE
PREDNISOLONE SODIUM PHOSPHATE
PREDNISOLONE VALEROACETATE
PREDNISOLONE; TETRYZOLINE HYDROCHLORIDE
   and mixtures thereof, wherein the patient receive an amount of the compound according to formula (I) of 20 mg.

In one embodiment, the CYP3A4 inducer of unspecified potency and/or a combination that comprises at least one CYP3A4 inducer of unspecified potency is selected from:
CYPROTERONE
LIGUSTRAZINE HYDROCHLORIDE
LIGUSTRAZINE HYDROCHLORIDE; SALVIA MILTIORRHIZA ROOT WITH RHIZOME
LIGUSTRAZINE PHOSPHATE
PREDNISOLONE
PREDNISOLONE ACETATE
PREDNISOLONE SODIUM PHOSPHATE
PREDNISOLONE STEAGLATE
PREDNISOLONE VALEROACETATE
   and mixtures thereof, wherein the patient receive an amount of the compound according to formula (I) of 40 mg.

Examples, but not limited to, weak CYP3A4 inducers can be alogliptin, pioglitazone, aprepitant, clobazam, metformin, oxcarbazepine, prednisone, quercetin.

In one embodiment, the weak CYP3A4 inducer and/or a combination that comprises at least one weak CYP3A4 inducer is selected from:
ALOGLIPTIN BENZOATE; PIOGLITAZONE HYDROCHLORIDE
APREPITANT
CLOBAZAM
METFORMIN HYDROCHLORIDE; PIOGLITAZONE HYDROCHLORIDE
METFORMIN; PIOGLITAZONE
OXCARBAZEPINE
PIOGLITAZONE
PIOGLITAZONE HYDROCHLORIDE
PREDNISONE
PREDNISONE ACETATE
QUERCETIN
   and mixtures thereof. In one embodiment thereof, the patient receives an amount of the compound according to formula (I) of 5 to 80 mg. In one embodiment thereof, the patient receives an amount of the compound according to formula (I) of 5 mg. In one embodiment thereof, the patient receives an amount of the compound according to formula (I) of 10 mg. In one embodiment thereof, the patient receives an amount of the compound according to formula (I) of 20 mg. In one embodiment thereof, the patient receives an amount of the compound according to formula (I) of 30 mg. In one embodiment thereof, the patient receives an amount of the compound according to formula (I) of 40 mg. In one embodiment thereof, the patient receives an amount of the compound according to formula (I) of 60 mg. In one embodiment thereof, the patient receives an amount of the compound according to formula (I) of 80 mg. The amount of the compound according to formula (I) in these embodiments can be adjusted based on the CYP3A4 inducer. The adjustment can be up-titration, down-titration, maintenance or interruption of the treatment with the compound according to formula (I). Multiple embodiments of dose adjustments are described in detail below. These can be applied based on the co-medication with a CYP3A4 inducer. These can also be applied based on the amount and/or type of the CYP3A4 inducer the patient receives.

In one embodiment, the weak CYP3A4 inducer and/or a combination that comprises at least one weak CYP3A4 inducer is selected from:
PREDNISONE
   wherein the patient receives an amount of the compound according to formula (I) of 5 mg.

In one embodiment, the weak CYP3A4 inducer and/or a combination that comprises at least one weak CYP3A4 inducer is selected from:
ALOGLIPTIN BENZOATE; PIOGLITAZONE HYDROCHLORIDE
METFORMIN HYDROCHLORIDE; PIOGLITAZONE HYDROCHLORIDE
OXCARBAZEPINE
PIOGLITAZONE
PIOGLITAZONE HYDROCHLORIDE
PREDNISONE
PREDNISONE ACETATE
QUERCETIN
   and mixtures thereof, wherein the patient receives an amount of the compound according to formula (I) of 10 mg.

In one embodiment, the weak CYP3A4 inducer and/or a combination that comprises at least one weak CYP3A4 inducer is selected from:
ALOGLIPTIN BENZOATE; PIOGLITAZONE HYDROCHLORIDE
APREPITANT
CLOBAZAM
METFORMIN HYDROCHLORIDE; PIOGLITAZONE HYDROCHLORIDE
OXCARBAZEPINE
PIOGLITAZONE
PIOGLITAZONE HYDROCHLORIDE
PREDNISONE
PREDNISONE ACETATE
QUERCETIN
   and mixtures thereof, wherein the patient receives an amount of the compound according to formula (I) of 20 mg.

In one embodiment, the weak CYP3A4 inducer and/or a combination that comprises at least one weak CYP3A4 inducer is selected from:
METFORMIN HYDROCHLORIDE; PIOGLITAZONE HYDROCHLORIDE
METFORMIN; PIOGLITAZONE
OXCARBAZEPINE
PIOGLITAZONE
PIOGLITAZONE HYDROCHLORIDE
PREDNISONE
PREDNISONE ACETATE
QUERCETIN
   and mixtures thereof, wherein the patient receives an amount of the compound according to formula (I) of 40 mg.

In one embodiment, the patient further receives a CYP3A4 inhibitor. The patient can receive the CYP3A4 inhibitor prior and/or during the treatment. In one embodiment, the patient further receives a weak CYP3A4 inhibitor. In one embodiment, the patient further receives a moderate CYP3A4 inhibitor. In one embodiment, the patient further receives a strong CYP3A4 inhibitor. Inhibitors of CYP3A4 are classified by potency. A strong inhibitor causes at least a 5-fold increase in the plasma AUC values, or equal to or corresponding to more than 80% decrease in clearance, of CYP3A4 sensitive (index) substrate of a given metabolic pathway. A moderate inhibitor causes at least a 2-fold but less than 5-fold increase in the plasma AUC values of sensitive index substrates of a given metabolic pathway or corresponding to 50-80% decrease in clearance. A weak inhibitor causes at least a 1.25-fold but less than 2-fold increase in the plasma AUC values or corresponding to 20-50% decrease in clearance.

The above definitions of strong and moderate CYP3A4 inhibitors come from FDA Guidance. See Clinical Drug Interaction Studies—Cytochrome P450 Enzyme- and Transporter-Mediated Drug Interactions Guidance for Industry, U.S. Department of Health and Human Services, Food and Drug Administration, Center for Drug Evaluation and Research (CDER), January 2020, (https://www.fda.gov/Drugs/GuidanceComplianceRegulatoryInformation/Guidances/default.htm) accessed Jan. 4, 2024 and incorporated herein by reference. Other sources may use a different classification system for strong and moderate inducers resulting in some agents being classified differently.

Examples, but not limited to, strong CYP3A4 inhibitors can be boceprevir, protease inhibitors (ritonavir [and combination drugs thereof], indinavir, nelfinavir, saquinavir, amprenavir, atazanavir, darunavir), cobicistat, clarithromycin, telithromycin, nefazodone, ceritinib, mibefradil, ribociclib, tucatinib, chloramphenicol (antibiotic), some azole antifungals (ketoconazole, itraconazole, posaconazole, voriconazole), green tea extract, grape seed extract, dillapiole, apigenin, Artemisia annua, aluminium chlorhydrate, micoconazol, hydrocortisone, josamycin, mazipredone, nirmatrelvir, adagrasib, conivaptan, delavirdine, ensitrelvir, grapefruit, idelalisib, indinavir, levoketoconazole, lonafarnib, lopinavir, mifepristone, nelfinavir, saquinavir, telaprevir, tipranavir, and/or troleandomycin.

In one embodiment, the strong CYP3A4 inhibitor and/or a combination that comprises at least one strong CYP3A4 inhibitor is selected from:
ALUMINIUM CHLOROHYDRATE; MICONAZOLE
CLARITHROMYCIN
CLARITHROMYCIN LACTOBIONATE
HYDROCORTISONE; MICONAZOLE
HYDROCORTISONE; MICONAZOLE NITRATE
ITRACONAZOLE
JOSAMYCIN
KETOCONAZOLE
MAZIPREDONE HYDROCHLORIDE; MICONAZOLE NITRATE
MICONAZOLE
MICONAZOLE NITRATE
NIRMATRELVIR; RITONAVIR
RITONAVIR
VORICONAZOLE
   and mixtures thereof. In one embodiment thereof, the patient receives an amount of the compound according to formula (I) of 0.5 to 80 mg. In one embodiment thereof, the patient receives an amount of the compound according to formula (I) of 0.5 mg. In one embodiment thereof, the patient receives an amount of the compound according to formula (I) of 1.25 mg. In one embodiment thereof, the patient receives an amount of the compound according to formula (I) of 2.5 mg. In one embodiment thereof, the patient receives an amount of the compound according to formula (I) of 5 mg. In one embodiment thereof, the patient receives an amount of the compound according to formula (I) of 10 mg. In one embodiment thereof, the patient receives an amount of the compound according to formula (I) of 20 mg. In one embodiment thereof, the patient receives an amount of the compound according to formula (I) of 30 mg. In one embodiment thereof, the patient receives an amount of the compound according to formula (I) of 40 mg. In one embodiment thereof, the patient receives an amount of the compound according to formula (I) of 60 mg. In one embodiment thereof, the patient receives an amount of the compound according to formula (I) of 80 mg. The amount of the compound according to formula (I) in these embodiments can be adjusted based on the CYP3A4 inhibitor. The adjustment can be up-titration, down-titration, maintenance or interruption of the treatment with the compound according to formula (I). Multiple embodiments of dose adjustments are described in detail below. These can be applied based on the co-medication with a CYP3A4 inhibitor. These can also be applied based on the amount and/or type of the CYP3A4 inhibitor the patient receives.

In one embodiment, the strong CYP3A4 inhibitor and/or a combination that comprises at least one strong CYP3A4 inhibitor is selected from:
ALUMINIUM CHLOROHYDRATE; MICONAZOLE
CLARITHROMYCIN
ITRACONAZOLE
KETOCONAZOLE
MICONAZOLE NITRATE
RITONAVIR
VORICONAZOLE
   and mixtures thereof, wherein the patient receives an amount of the compound according to formula (I) of 10 mg.

In one embodiment, the strong CYP3A4 inhibitor and/or a combination that comprises at least one strong CYP3A4 inhibitor is selected from:
ALUMINIUM CHLOROHYDRATE; MICONAZOLE
CLARITHROMYCIN
CLARITHROMYCIN LACTOBIONATE
HYDROCORTISONE; MICONAZOLE
HYDROCORTISONE; MICONAZOLE NITRATE
ITRACONAZOLE
KETOCONAZOLE
MAZIPREDONE HYDROCHLORIDE; MICONAZOLE NITRATE
MICONAZOLE
MICONAZOLE NITRATE
NIRMATRELVIR; RITONAVIR
RITONAVIR
   and mixtures thereof, wherein the patients receive an amount of the compound according to formula (I) of 20 mg.

In one embodiment, the strong CYP3A4 inhibitor and/or a combination that comprises at least one strong CYP3A4 inhibitor is selected from:
CLARITHROMYCIN
HYDROCORTISONE; MICONAZOLE
HYDROCORTISONE; MICONAZOLE NITRATE
ITRACONAZOLE
KETOCONAZOLE
MICONAZOLE
NIRMATRELVIR; RITONA VIR
RITONA VIR
VORICONAZOLE
   and mixtures thereof, wherein the patient receives an amount of the compound according to formula (I) of 40 mg.

Examples, but not limited to, moderate CYP3A4 inhibitors can be aprepitant, ciprofloxacin, conivaptan, crizotinib, rutin, tofisopam, some calcium channel blockers (verapamil, diltiazem), some azole antifungals (fluconazole, miconazole), bergamottin, cyclosporine, donedarone, fluvoxamine, imatinib, valerian, and/or erythromycin.

In one embodiment, the moderate CYP3A4 inhibitor and/or a combination that comprises at least one moderate CYP3A4 inhibitor is selected from:
APREPITANT
ATAZANAVIR
ATAZANAVIR SULFATE
BETAMETHASONE DIPROPIONATE; CLOTRIMAZOLE
BETAMETHASONE DIPROPIONATE; CLOTRIMAZOLE; GENTAMICIN SULFATE
BETAMETHASONE DIPROPIONATE; CLOTRIMAZOLE; NEOMYCIN SULFATE
BETAMETHASONE; CHLORAMPHENICOL
BETAMETHASONE; CLOTRIMAZOLE
CHLORAMPHENICOL
CHLORAMPHENICOL PALMITATE
CHLORAMPHENICOL; COLLAGENASE
CHLORAMPHENICOL; DEXAMETHASONE
CHLORAMPHENICOL; METHYLURACIL
CICLOSPORIN
CIMETIDINE
CIPROFLOXACIN
CIPROFLOXACIN HYDROCHLORIDE
CIPROFLOXACIN HYDROCHLORIDE MONOHYDRATE
CIPROFLOXACIN HYDROCHLORIDE; DEXAMETHASONE
CIPROFLOXACIN HYDROCHLORIDE; FLUOCINOLONE ACETONIDE
CIPROFLOXACIN HYDROCHLORIDE; TINIDAZOLE
CIPROFLOXACIN LACTATE
CIPROFLOXACIN; FLUOCINOLONE ACETONIDE
CLOTRIMAZOLE
CLOTRIMAZOLE; HEXAMIDINE ISETIONATE; PREDNISOLONE ACETATE
CLOTRIMAZOLE; HYDROCORTISONE
CLOTRIMAZOLE; HYDROCORTISONE; ZINC OXIDE
COLISTIMETHATE SODIUM; ERYTHROMYCIN LACTOBIONATE
COLISTIN; ERYTHROMYCIN LACTOBIONATE
DILTIAZEM
DILTIAZEM HYDROCHLORIDE
DRONEDARONE
DRONEDARONE HYDROCHLORIDE
ERYTHROMYCIN
ERYTHROMYCIN ETHYLSUCCINATE
ERYTHROMYCIN LACTOBIONATE
ERYTHROMYCIN PROPIONATE
FLUCONAZOLE
FLUVOXAMINE
IMATINIB MESILATE
TOFISOPAM
TRANDOLAPRIL; VERAPAMIL HYDROCHLORIDE
VERAPAMIL
VERAPAMIL HYDROCHLORIDE
   and mixtures thereof. In one embodiment thereof, the patient receives an amount of the compound according to formula (I) of 5 to 80 mg. In one embodiment thereof, the patient receives an amount of the compound according to formula (I) of 5 mg. In one embodiment thereof, the patient receives an amount of the compound according to formula (I) of 10 mg. In one embodiment thereof, the patient receives an amount of the compound according to formula (I) of 20 mg. In one embodiment thereof, the patient receives an amount of the compound according to formula (I) of 30 mg. In one embodiment thereof, the patient receives an amount of the compound according to formula (I) of 40 mg. In one embodiment thereof, the patient receives an amount of the compound according to formula (I) of 60 mg. In one embodiment thereof, the patient receives an amount of the compound according to formula (I) of 80 mg. The amount of the compound according to formula (I) in these embodiments can be adjusted based on the CYP3A4 inhibitor. The adjustment can be up-titration, down-titration, maintenance or interruption of the treatment with the compound according to formula (I). Multiple embodiments of dose adjustments are described in detail below. These can be applied based on the co-medication with a CYP3A4 inhibitor. These can also be applied based on the amount and/or type of the CYP3A4 inhibitor the patient receives.

In one embodiment, the moderate CYP3A4 inhibitor and/or a combination that comprises at least one moderate CYP3A4 inhibitor is selected from:
BETAMETHASONE; CHLORAMPHENICOL
BETAMETHASONE; CLOTRIMAZOLE
CHLORAMPHENICOL
CICLOSPORIN
CIMETIDINE
CIPROFLOXACIN
CIPROFLOXACIN HYDROCHLORIDE
CLOTRIMAZOLE
DILTIAZEM
DILTIAZEM HYDROCHLORIDE
DRONEDARONE
DRONEDARONE HYDROCHLORIDE
ERYTHROMYCIN
ERYTHROMYCIN PROPIONATE
FLUCONAZOLE
FLUVOXAMINE
TOFISOPAM
TRANDOLAPRIL; VERAPAMIL HYDROCHLORIDE
VERAPAMIL
VERAPAMIL HYDROCHLORIDE
   and mixtures thereof, wherein the patient receives an amount of the compound according to formula (I) of 10 mg.

In one embodiment, the moderate CYP3A4 inhibitor and/or a combination that comprises at least one moderate CYP3A4 inhibitor is selected from:
APREPITANT
BETAMETHASONE DIPROPIONATE; CLOTRIMAZOLE
BETAMETHASONE DIPROPIONATE; CLOTRIMAZOLE; NEOMYCIN SULFATE
BETAMETHASONE; CLOTRIMAZOLE
CHLORAMPHENICOL
CHLORAMPHENICOL PALMITATE
CHLORAMPHENICOL; COLLAGENASE
CHLORAMPHENICOL; DEXAMETHASONE
CICLOSPORIN
CIMETIDINE
CIPROFLOXACIN
CIPROFLOXACIN HYDROCHLORIDE
CIPROFLOXACIN HYDROCHLORIDE; FLUOCINOLONE ACETONIDE
CIPROFLOXACIN HYDROCHLORIDE; TINIDAZOLE
CIPROFLOXACIN LACTATE
CIPROFLOXACIN; FLUOCINOLONE ACETONIDE
CLOTRIMAZOLE
COLISTIMETHATE SODIUM; ERYTHROMYCIN LACTOBIONATE
COLISTIN; ERYTHROMYCIN LACTOBIONATE
DILTIAZEM
DILTIAZEM HYDROCHLORIDE
DRONEDARONE HYDROCHLORIDE
ERYTHROMYCIN
ERYTHROMYCIN ETHYLSUCCINATE
FLUCONAZOLE
FLUVOXAMINE
IMATINIB MESILATE
TOFISOPAM
VERAPAMIL
VERAPAMIL HYDROCHLORIDE
   and mixtures thereof, wherein the patient receives an amount of the compound according to formula (I) of 20 mg.

In one embodiment, the moderate CYP3A4 inhibitor and/or a combination that comprises at least one moderate CYP3A4 inhibitor is selected from:
BETAMETHASONE DIPROPIONATE; CLOTRIMAZOLE
BETAMETHASONE DIPROPIONATE; CLOTRIMAZOLE; NEOMYCIN SULFATE
CHLORAMPHENICOL
CHLORAMPHENICOL; COLLAGENASE
CHLORAMPHENICOL; METHYLURACIL
CICLOSPORIN
CIPROFLOXACIN
CIPROFLOXACIN HYDROCHLORIDE
CIPROFLOXACIN HYDROCHLORIDE MONOHYDRATE
CIPROFLOXACIN HYDROCHLORIDE; DEXAMETHASONE
CIPROFLOXACIN HYDROCHLORIDE; FLUOCINOLONE ACETONIDE
CIPROFLOXACIN LACTATE
CLOTRIMAZOLE
CLOTRIMAZOLE; HYDROCORTISONE
COLISTIMETHATE SODIUM; ERYTHROMYCIN LACTOBIONATE
COLISTIN; ERYTHROMYCIN LACTOBIONATE
DILTIAZEM
DILTIAZEM HYDROCHLORIDE
DRONEDARONE HYDROCHLORIDE
ERYTHROMYCIN
FLUCONAZOLE
VERAPAMIL
VERAPAMIL HYDROCHLORIDE
   and mixtures thereof, wherein the patient receives an amount of the compound according to formula (I) of 40 mg.

Examples, but not limited to, weak CYP3A4 inhibitors can be berberine, buprenorphine, cafestol, cilostazol, cimetidine, fosaprepitant, lomitapide, orphenadrine, omeprazole, quercetin, ranitidine, ranolazine, tacrolimus, ticagrelor, valproic acid, amlodipine, azithromycin, cimetidine, fluvoxamine, and/or amiodarone.

In one embodiment, the weak CYP3A4 inhibitor and/or a combination that comprises at least one weak CYP3A4 inhibitor is selected from:
ACETYLSALICYLIC ACID; ATORVASTATIN
ACETYLSALICYLIC ACID; ATORVASTATIN CALCIUM
ACETYLSALICYLIC ACID; ATORVASTATIN CALCIUM; CLOPIDOGREL BISULFATE
ACETYLSALICYLIC ACID; ATORVASTATIN CALCIUM; RAMIPRIL
ALLIUM SATIVUM BULB; BLUMEA BALSAMIFERA LEAF; CRATAEGUS PINNATIFIDA FRUIT; ERIGERON BREVISCAPUS WHOLE PLANT; GINKGO BILOBA LEAF; GYNOSTEMMA PENTAPHYLLUM HERB; PANAX NOTOGINSENG ROOT WITH RHIZOME; SALVIA MILTIORRHIZA ROOT WITH RHIZOME
ALLIUM SPP. BULB; BUXUS SPP. LEAF WITH TWIG; CINNAMOMUM MIGAO FRUIT; GINKGO BILOBA LEAF; SALVIA MILTIORRHIZA ROOT WITH RHIZOME
ALPRAZOLAM
AMBROXOL HYDROCHLORIDE; ROXITHROMYCIN
AMIODARONE
AMIODARONE HYDROCHLORIDE
AMLODIPINE
AMLODIPINE BENZOATE
AMLODIPINE BESILATE
AMLODIPINE BESILATE; ATORVASTATIN CALCIUM
AMLODIPINE BESILATE; ATORVASTATIN CALCIUM TRIHYDRATE; PERINDOPRIL ARGININE
AMLODIPINE BESILATE; ATORVASTATIN CALCIUM; PERINDOPRIL ARGININE
AMLODIPINE BESILATE; ATORVASTATIN L-LYSINE
AMLODIPINE BESILATE; AZILSARTAN
AMLODIPINE BESILATE; BENAZEPRIL HYDROCHLORIDE
AMLODIPINE BESILATE; BISOPROLOL FUMARATE
AMLODIPINE BESILATE; CANDESARTAN CILEXETIL
AMLODIPINE BESILATE; HYDROCHLOROTHIAZIDE; OLMESARTAN MEDOXOMIL
AMLODIPINE BESILATE; HYDROCHLOROTHIAZIDE; VALSARTAN
AMLODIPINE BESILATE; INDAPAMIDE
AMLODIPINE BESILATE; INDAPAMIDE; PERINDOPRIL ARGININE
AMLODIPINE BESILATE; INDAPAMIDE; PERINDOPRIL ERBUMINE
AMLODIPINE BESILATE; IRBESARTAN
AMLODIPINE BESILATE; LISINOPRIL
AMLODIPINE BESILATE; LISINOPRIL DIHYDRATE
AMLODIPINE BESILATE; LOSARTAN POTASSIUM
AMLODIPINE BESILATE; OLMESARTAN MEDOXOMIL
AMLODIPINE BESILATE; PERINDOPRIL ARGININE
AMLODIPINE BESILATE; PERINDOPRIL ERBUMINE
AMLODIPINE BESILATE; PERINDOPRIL TOSILATE
AMLODIPINE BESILATE; RAMIPRIL
AMLODIPINE BESILATE; ROSUVASTATIN CALCIUM; TELMISARTAN
AMLODIPINE BESILATE; TELMISARTAN
AMLODIPINE BESILATE; VALSARTAN
AMLODIPINE CAMSILATE
AMLODIPINE MALEATE
AMLODIPINE MESILATE
AMLODIPINE; ATORVASTATIN CALCIUM
AMLODIPINE; BENAZEPRIL HYDROCHLORIDE
AMLODIPINE; HYDROCHLOROTHIAZIDE; OLMESARTAN
AMLODIPINE; HYDROCHLOROTHIAZIDE; VALSARTAN
AMLODIPINE; INDAPAMIDE
AMLODIPINE; PERINDOPRIL
AMLODIPINE; RAMIPRIL
AMLODIPINE; VALSARTAN
ASTAXANTHIN; BERBERINE; FOLIC ACID; MONASCUS PURPUREUS; POLICOSANOL; UBIDECARENONE
ATORVASTATIN
ATORVASTATIN CALCIUM
ATORVASTATIN CALCIUM TRIHYDRATE
ATORVASTATIN CALCIUM TRIHYDRATE; EZETIMIBE
ATORVASTATIN CALCIUM; EZETIMIBE
ATORVASTATIN CALCIUM; PERINDOPRIL ARGININE
ATORVASTATIN; EZETIMIBE
BACOPA MONNIERI; CITICOLINE SODIUM; GINKGO BILOBA; PHOSPHATIDYL SERINE; SALVIA OFFICINALIS; THIOCTIC ACID
BERBERINE
BERBERINE CHLORIDE HYDRATE
BERBERINE HYDROCHLORIDE
BERBERINE HYDROCHLORIDE; DOLOMIAEA COSTUS ROOT; TETRADIUM RUTICARPUM FRUIT
BERBERINE HYDROCHLORIDE; RHEUM SPP. ROOT WITH RHIZOME; SCUTELLARIA BAICALENSIS ROOT
BERBERINE; CINNAMOMUM VERUM; GYMNEMA SYLVESTRE; LAGERSTROEMIA SPECIOSA; PICRASMA EXCELSA; SYZYGIUM CUMINI; URTICA DIOICA; VACCINIUM MYRTILLUS
BICALUTAMIDE
BORNEOL; GINKGO BILOBA LEAF; GYNOSTEMMA PENTAPHYLLUM WHOLE PLANT; SALVIA YUNNANENSIS ROOT
CAFFEINE; CHLORZOXAZONE; PARACETAMOL; THIAMINE
CHLORZOXAZONE
CHLORZOXAZONE; PARACETAMOL
CHOLINE; GINKGO BILOBA LEAF
CILOSTAZOL
CYANOCOBALAMIN; FISH OIL; FOLIC ACID; GINKGO BILOBA LEAF; PHOSPHATIDYL SERINE; TOCOPHEROL
DIPYRIDAMOLE; GINKGO BILOBA LEAF EXTRACT
ELEUTHEROCOCCUS SENTICOSUS; EUPHRASIA OFFICINALIS; GINKGO BILOBA; VACCINIUM MYRTILLUS
EVEROLIMUS
FLUOXETINE
FLUOXETINE HYDROCHLORIDE
GINKGO BILOBA
GINKGO BILOBA EXTRACT
GINKGO BILOBA LEAF
GINKGO BILOBA LEAF EXTRACT
GINKGO BILOBA LEAF; LIGUSTRAZINE PHOSPHATE
GINKGO BILOBA; GLUCOSAMINE
ISONIAZID
LEVAMLODIPINE
LEVAMLODIPINE BESILATE
LEVAMLODIPINE MALEATE
PROPIVERINE
PROPIVERINE HYDROCHLORIDE
RANITIDINE
RANITIDINE HYDROCHLORIDE
RANITIDINE HYDROCHLORIDE; SUCRALFATE; TRIPOTASSIUM DICITRATOBISMUTHATE
RANOLAZINE
ROXITHROMYCIN
TACROLIMUS
TACROLIMUS MONOHYDRATE
TICAGRELOR
TOLVAPTAN
TOLVAPTAN DISODIUM PHOSPHATE
   and mixtures thereof. In one embodiment thereof, the patient receives an amount of the compound according to formula (I) of 5 to 80 mg. In one embodiment thereof, the patient receives an amount of the compound according to formula (I) of 5 mg. In one embodiment thereof, the patient receives an amount of the compound according to formula (I) of 10 mg. In one embodiment thereof, the patient receives an amount of the compound according to formula (I) of 20 mg. In one embodiment thereof, the patient receives an amount of the compound according to formula (I) of 30 mg. In one embodiment thereof, the patient receives an amount of the compound according to formula (I) of 40 mg. In one embodiment thereof, the patient receives an amount of the compound according to formula (I) of 60 mg. In one embodiment thereof, the patient receives an amount of the compound according to formula (I) of 80 mg. The amount of the compound according to formula (I) in these embodiments can be adjusted based on the CYP3A4 inhibitor. The adjustment can be up-titration, down-titration, maintenance or interruption of the treatment with the compound according to formula (I). Multiple embodiments of dose adjustments are described in detail below. These can be applied based on the co-medication with a CYP3A4 inhibitor. These can also be applied based on the amount and/or type of the CYP3A4 inhibitor the patient receives.

In one embodiment, the weak CYP3A4 inhibitor and/or a combination that comprises at least one weak CYP3A4 inhibitor is selected from:
ATORVASTATIN
   and mixtures thereof, wherein the patient receives an amount of the compound according to formula (I) of 5 mg.

In one embodiment, the weak CYP3A4 inhibitor and/or a combination that comprises at least one weak CYP3A4 inhibitor is selected from:
ACETYLSALICYLIC ACID; ATORVASTATIN
ACETYLSALICYLIC ACID; ATORVASTATIN CALCIUM; CLOPIDOGREL BISULFATE
ALPRAZOLAM
AMIODARONE
AMIODARONE HYDROCHLORIDE
AMLODIPINE
AMLODIPINE BESILATE
AMLODIPINE BESILATE; ATORVASTATIN CALCIUM
AMLODIPINE BESILATE; ATORVASTATIN CALCIUM TRIHYDRATE; PERINDOPRIL ARGININE
AMLODIPINE BESILATE; AZILSARTAN
AMLODIPINE BESILATE; BENAZEPRIL HYDROCHLORIDE
AMLODIPINE BESILATE; HYDROCHLOROTHIAZIDE; OLMESARTAN MEDOXOMIL
AMLODIPINE BESILATE; HYDROCHLOROTHIAZIDE; VALSARTAN
AMLODIPINE BESILATE; INDAPAMIDE
AMLODIPINE BESILATE; INDAPAMIDE; PERINDOPRIL ARGININE
AMLODIPINE BESILATE; INDAPAMIDE; PERINDOPRIL ERBUMINE
AMLODIPINE BESILATE; IRBESARTAN
AMLODIPINE BESILATE; LISINOPRIL
AMLODIPINE BESILATE; LISINOPRIL DIHYDRATE
AMLODIPINE BESILATE; LOSARTAN POTASSIUM
AMLODIPINE BESILATE; OLMESARTAN MEDOXOMIL
AMLODIPINE BESILATE; PERINDOPRIL ARGININE
AMLODIPINE BESILATE; PERINDOPRIL ERBUMINE
AMLODIPINE BESILATE; PERINDOPRIL TOSILATE
AMLODIPINE BESILATE; RAMIPRIL
AMLODIPINE BESILATE; ROSUVASTATIN CALCIUM; TELMISARTAN
AMLODIPINE BESILATE; TELMISARTAN
AMLODIPINE BESILATE; VALSARTAN
AMLODIPINE MALEATE
AMLODIPINE MESILATE
AMLODIPINE; ATORVASTATIN CALCIUM
AMLODIPINE; INDAPAMIDE
AMLODIPINE; VALSARTAN
ATORVASTATIN
ATORVASTATIN CALCIUM
ATORVASTATIN CALCIUM TRIHYDRATE
ATORVASTATIN CALCIUM TRIHYDRATE; EZETIMIBE
ATORVASTATIN CALCIUM; EZETIMIBE
ATORVASTATIN; EZETIMIBE
BERBERINE HYDROCHLORIDE; DOLOMIAEA COSTUS ROOT; TETRADIUM RUTICARPUM FRUIT
BICALUTAMIDE
BORNEOL; GINKGO BILOBA LEAF; GYNOSTEMMA PENTAPHYLLUM WHOLE PLANT; SALVIA YUNNANENSIS ROOT
CHLORZOXAZONE
CHLORZOXAZONE; PARACETAMOL
CILOSTAZOL
EVEROLIMUS
FLUOXETINE
FLUOXETINE HYDROCHLORIDE
GINKGO BILOBA
GINKGO BILOBA EXTRACT
GINKGO BILOBA LEAF EXTRACT
GINKGO BILOBA; GLUCOSAMINE
ISONIAZID
LEVAMLODIPINE
LEVAMLODIPINE BESILATE
LEVAMLODIPINE MALEATE
PROPIVERINE HYDROCHLORIDE
RANITIDINE
RANITIDINE HYDROCHLORIDE
RANOLAZINE
ROXITHROMYCIN
TACROLIMUS
TACROLIMUS MONOHYDRATE
TICAGRELOR
TOLVAPTAN
TOLVAPTAN DISODIUM PHOSPHATE
   and mixtures thereof, wherein the patient receives an amount of the compound according to formula (I) of 10 mg.

In one embodiment, the weak CYP3A4 inhibitor and/or a combination that comprises at least one weak CYP3A4 inhibitor is selected from:
ACETYLSALICYLIC ACID; ATORVASTATIN
ACETYLSALICYLIC ACID; ATORVASTATIN CALCIUM; CLOPIDOGREL BISULFATE
ACETYLSALICYLIC ACID; ATORVASTATIN CALCIUM; RAMIPRIL
ALPRAZOLAM
AMIODARONE
AMIODARONE HYDROCHLORIDE
AMLODIPINE
AMLODIPINE BENZOATE
AMLODIPINE BESILATE
AMLODIPINE BESILATE; ATORVASTATIN CALCIUM
AMLODIPINE BESILATE; ATORVASTATIN CALCIUM TRIHYDRATE; PERINDOPRIL ARGININE
AMLODIPINE BESILATE; ATORVASTATIN CALCIUM; PERINDOPRIL ARGININE
AMLODIPINE BESILATE; ATORVASTATIN L-LYSINE
AMLODIPINE BESILATE; BENAZEPRIL HYDROCHLORIDE
AMLODIPINE BESILATE; BISOPROLOL FUMARATE
AMLODIPINE BESILATE; HYDROCHLOROTHIAZIDE; OLMESARTAN MEDOXOMIL
AMLODIPINE BESILATE; HYDROCHLOROTHIAZIDE; VALSARTAN
AMLODIPINE BESILATE; INDAPAMIDE
AMLODIPINE BESILATE; INDAPAMIDE; PERINDOPRIL ARGININE
AMLODIPINE BESILATE; INDAPAMIDE; PERINDOPRIL ERBUMINE
AMLODIPINE BESILATE; IRBESARTAN
AMLODIPINE BESILATE; LISINOPRIL
AMLODIPINE BESILATE; LISINOPRIL DIHYDRATE
AMLODIPINE BESILATE; LOSARTAN POTASSIUM
AMLODIPINE BESILATE; OLMESARTAN MEDOXOMIL
AMLODIPINE BESILATE; PERINDOPRIL ARGININE
AMLODIPINE BESILATE; PERINDOPRIL ERBUMINE
AMLODIPINE BESILATE; PERINDOPRIL TOSILATE
AMLODIPINE BESILATE; RAMIPRIL
AMLODIPINE BESILATE; ROSUVASTATIN CALCIUM; TELMISARTAN
AMLODIPINE BESILATE; TELMISARTAN
AMLODIPINE BESILATE; VALSARTAN
AMLODIPINE CAMSILATE
AMLODIPINE MALEATE
AMLODIPINE MESILATE
AMLODIPINE; ATORVASTATIN CALCIUM
AMLODIPINE; HYDROCHLOROTHIAZIDE; OLMESARTAN
AMLODIPINE; HYDROCHLOROTHIAZIDE; VALSARTAN
AMLODIPINE; PERINDOPRIL
AMLODIPINE; VALSARTAN
ASTAXANTHIN; BERBERINE; FOLIC ACID; MONASCUS PURPUREUS; POLICOSANOL; UBIDECARENONE
ATORVASTATIN
ATORVASTATIN CALCIUM
ATORVASTATIN CALCIUM TRIHYDRATE
ATORVASTATIN CALCIUM TRIHYDRATE; EZETIMIBE
ATORVASTATIN CALCIUM; EZETIMIBE
ATORVASTATIN CALCIUM; PERINDOPRIL ARGININE
ATORVASTATIN; EZETIMIBE
BACOPA MONNIERI; CITICOLINE SODIUM; GINKGO BILOBA; PHOSPHATIDYL SERINE; SALVIA OFFICINALIS; THIOCTIC ACID
BERBERINE CHLORIDE HYDRATE
BERBERINE HYDROCHLORIDE
BERBERINE; CINNAMOMUM VERUM; GYMNEMA SYLVESTRE; LAGERSTROEMIA SPECIOSA; PICRASMA EXCELSA; SYZYGIUM CUMINI; URTICA DIOICA; VACCINIUM MYRTILLUS
BICALUTAMIDE
BORNEOL; GINKGO BILOBA LEAF; GYNOSTEMMA PENTAPHYLLUM WHOLE PLANT; SALVIA YUNNANENSIS ROOT
CAFFEINE; CHLORZOXAZONE; PARACETAMOL; THIAMINE
CHLORZOXAZONE
CHLORZOXAZONE; PARACETAMOL
CHOLINE; GINKGO BILOBA LEAF
CILOSTAZOL
ELEUTHEROCOCCUS SENTICOSUS; EUPHRASIA OFFICINALIS; GINKGO BILOBA; VACCINIUM MYRTILLUS
EVEROLIMUS
FLUOXETINE
FLUOXETINE HYDROCHLORIDE
GINKGO BILOBA
GINKGO BILOBA EXTRACT
GINKGO BILOBA LEAF
GINKGO BILOBA LEAF EXTRACT
GINKGO BILOBA; GLUCOSAMINE
ISONIAZID
LEVAMLODIPINE
LEVAMLODIPINE BESILATE
LEVAMLODIPINE MALEATE
PROPIVERINE HYDROCHLORIDE
RANITIDINE
RANITIDINE HYDROCHLORIDE
RANOLAZINE
ROXITHROMYCIN
TACROLIMUS
TACROLIMUS MONOHYDRATE
TICAGRELOR
TOLVAPTAN
TOLVAPTAN DISODIUM PHOSPHATE
   and mixtures thereof, wherein the patient receives an amount of the compound according to formula (I) of 20 mg.

In one embodiment, the weak CYP3A4 inhibitor and/or a combination that comprises at least one weak CYP3A4 inhibitor is selected from:
ALPRAZOLAM
ATORVASTATIN; EZETIMIBE
   and mixtures thereof, wherein the patient receives an amount of the compound according to formula (I) of 30 mg.

In one embodiment, the weak CYP3A4 inhibitor and/or a combination that comprises at least one weak CYP3A4 inhibitor is selected from:
ACETYLSALICYLIC ACID; ATORVASTATIN CALCIUM; RAMIPRIL
ALLIUM SPP. BULB; BUXUS SPP. LEAF WITH TWIG; CINNAMOMUM MIGAO FRUIT; GINKGO BILOBA LEAF; SALVIA MILTIORRHIZA ROOT WITH RHIZOME
ALPRAZOLAM
AMBROXOL HYDROCHLORIDE; ROXITHROMYCIN
AMIODARONE
AMIODARONE HYDROCHLORIDE
AMLODIPINE
AMLODIPINE BESILATE
AMLODIPINE BESILATE; ATORVASTATIN CALCIUM
AMLODIPINE BESILATE; ATORVASTATIN CALCIUM; PERINDOPRIL ARGININE
AMLODIPINE BESILATE; ATORVASTATIN L-LYSINE
AMLODIPINE BESILATE; BENAZEPRIL HYDROCHLORIDE
AMLODIPINE BESILATE; BISOPROLOL FUMARATE
AMLODIPINE BESILATE; CANDESARTAN CILEXETIL
AMLODIPINE BESILATE; HYDROCHLOROTHIAZIDE; OLMESARTAN MEDOXOMIL
AMLODIPINE BESILATE; HYDROCHLOROTHIAZIDE; VALSARTAN
AMLODIPINE BESILATE; INDAPAMIDE
AMLODIPINE BESILATE; INDAPAMIDE; PERINDOPRIL ARGININE
AMLODIPINE BESILATE; INDAPAMIDE; PERINDOPRIL ERBUMINE
AMLODIPINE BESILATE; LISINOPRIL
AMLODIPINE BESILATE; LISINOPRIL DIHYDRATE
AMLODIPINE BESILATE; OLMESARTAN MEDOXOMIL
AMLODIPINE BESILATE; PERINDOPRIL ARGININE
AMLODIPINE BESILATE; PERINDOPRIL ERBUMINE
AMLODIPINE BESILATE; RAMIPRIL
AMLODIPINE BESILATE; TELMISARTAN
AMLODIPINE BESILATE; VALSARTAN
AMLODIPINE CAMSILATE
AMLODIPINE MALEATE
AMLODIPINE MESILATE
AMLODIPINE; ATORVASTATIN CALCIUM
AMLODIPINE; HYDROCHLOROTHIAZIDE; OLMESARTAN
AMLODIPINE; HYDROCHLOROTHIAZIDE; VALSARTAN
AMLODIPINE; RAMIPRIL
AMLODIPINE; VALSARTAN
ASTAXANTHIN; BERBERINE; FOLIC ACID; MONASCUS PURPUREUS; POLICOSANOL; UBIDECARENONE
ATORVASTATIN
ATORVASTATIN CALCIUM
ATORVASTATIN CALCIUM TRIHYDRATE
ATORVASTATIN CALCIUM; EZETIMIBE
ATORVASTATIN CALCIUM; PERINDOPRIL ARGININE
ATORVASTATIN; EZETIMIBE
BERBERINE
BERBERINE HYDROCHLORIDE
BERBERINE HYDROCHLORIDE; RHEUM SPP. ROOT WITH RHIZOME; SCUTELLARIA BAICALENSIS ROOT
BICALUTAMIDE
CHLORZOXAZONE
CHLORZOXAZONE; PARACETAMOL
CILOSTAZOL
CYANOCOBALAMIN; FISH OIL; FOLIC ACID; GINKGO BILOBA LEAF; PHOSPHATIDYL SERINE; TOCOPHEROL
ELEUTHEROCOCCUS SENTICOSUS; EUPHRASIA OFFICINALIS; GINKGO BILOBA; VACCINIUM MYRTILLUS
FLUOXETINE
FLUOXETINE HYDROCHLORIDE
GINKGO BILOBA
GINKGO BILOBA EXTRACT
GINKGO BILOBA LEAF
GINKGO BILOBA LEAF EXTRACT
LEVAMLODIPINE BESILATE
LEVAMLODIPINE MALEATE
PROPIVERINE
PROPIVERINE HYDROCHLORIDE
RANITIDINE
RANITIDINE HYDROCHLORIDE
RANITIDINE HYDROCHLORIDE; SUCRALFATE; TRIPOTASSIUM DICITRATOBISMUTHATE
RANOLAZINE
ROXITHROMYCIN
TACROLIMUS
TICAGRELOR
TOLVAPTAN
   and mixtures thereof, wherein the patient receives an amount of the compound according to formula (I) of 40 mg.

In one embodiment, the weak CYP3A4 inhibitor and/or a combination that comprises at least one weak CYP3A4 inhibitor is selected from:
ATORVASTATIN
   wherein the patient receives an amount of the compound according to formula (I) of 60 mg.

In one embodiment, the weak CYP3A4 inhibitor and/or a combination that comprises at least one weak CYP3A4 inhibitor is selected from:
AMIODARONE HYDROCHLORIDE
AMLODIPINE
AMLODIPINE BESILATE; HYDROCHLOROTHIAZIDE; VALSARTAN
ATORVASTATIN
ATORVASTATIN CALCIUM
ATORVASTATIN CALCIUM TRIHYDRATE
BICALUTAMIDE
TOLVAPTAN
   and mixtures thereof, wherein the patient receives an amount of the compound according to formula (I) of 80 mg.

Examples, but limited to, CYP3A4 inhibitors of unspecified potency can be bergaptol (a furocoumarin in citrus), cannabidiol, dithiocarbamate (functional group), flavonoids, mifepristone, norfloxacin, some non-nucleoside reverse-transcriptase inhibitors (delavirdine), gestodene, star fruit, milk thistle, niacin, nicotinic acid, niacinamide (nicotinamide), Vitamin B3 (complex), Ginkgo biloba, sesamin, piperine, isoniazid, serenoa.

In one embodiment, the CYP3A4 inhibitor of unspecified potency and/or a combination that comprises at least one CYP3A4 inhibitor of unspecified potency is selected from:
ALTIZIDE; SPIRONOLACTONE
BILOBALIDE; GINKGOLIDE A; GINKGOLIDE B; GINKGOLIDE C
BUTIZIDE; SPIRONOLACTONE
CAFFEINE; ERGOTAMINE TARTRATE; METAMIZOLE SODIUM
DESOGESTREL; ETHINYLESTRADIOL
ERGOTAMINE TARTRATE
ETHINYLESTRADIOL; NORGESTIMATE
FEXUPRAZAN HYDROCHLORIDE
FUROSEMIDE; SPIRONOLACTONE
HYDROCHLOROTHIAZIDE; SPIRONOLACTONE
MIDAZOLAM
MIDAZOLAM HYDROCHLORIDE
NORFLOXACIN
SERTRALINE
SERTRALINE HYDROCHLORIDE
SPIRONOLACTONE
SPIRONOLACTONE; TORASEMIDE
   and mixtures thereof. In one embodiment thereof, the patient receives an amount of the compound according to formula (I) of 5 to 80 mg In one embodiment thereof, the patient receives an amount of the compound according to formula (I) of 5 mg. In one embodiment thereof, the patient receives an amount of the compound according to formula (I) of 10 mg. In one embodiment thereof, the patient receives an amount of the compound according to formula (I) of 20 mg. In one embodiment thereof, the patient receives an amount of the compound according to formula (I) of 30 mg. In one embodiment thereof, the patient receives an amount of the compound according to formula (I) of 40 mg. In one embodiment thereof, the patient receives an amount of the compound according to formula (I) of 60 mg. In one embodiment thereof, the patient receives an amount of the compound according to formula (I) of 80 mg. The amount of the compound according to formula (I) in these embodiments can be adjusted based on the CYP3A4 inhibitor. The adjustment can be up-titration, down-titration, maintenance or interruption of the treatment with the compound according to formula (I). Multiple embodiments of dose adjustments are described in detail below. These can be applied based on the co-medication with a CYP3A4 inhibitor. These can also be applied based on the amount and/or type of the CYP3A4 inhibitor the patient receives.

In one embodiment, the CYP3A4 inhibitor of unspecified potency and/or a combination that comprises at least one CYP3A4 inhibitor of unspecified potency is selected from:

CAFFEINE; ERGOTAMINE TARTRATE; METAMIZOLE SODIUM
ERGOTAMINE TARTRATE
ETHINYLESTRADIOL; NORGESTIMATE
FEXUPRAZAN HYDROCHLORIDE
FUROSEMIDE; SPIRONOLACTONE
HYDROCHLOROTHIAZIDE; SPIRONOLACTONE
MIDAZOLAM
MIDAZOLAM HYDROCHLORIDE
NORFLOXACIN
SERTRALINE
SERTRALINE HYDROCHLORIDE
SPIRONOLACTONE
   and mixtures thereof, wherein the patient receives an amount of the compound according to formula (I) of 10 mg.

In one embodiment, the CYP3A4 inhibitor of unspecified potency and/or a combination that comprises at least one CYP3A4 inhibitor of unspecified potency is selected from:
BILOBALIDE; GINKGOLIDE A; GINKGOLIDE B; GINKGOLIDE C
CAFFEINE; ERGOTAMINE TARTRATE; METAMIZOLE SODIUM
ERGOTAMINE TARTRATE
FEXUPRAZAN HYDROCHLORIDE
FUROSEMIDE; SPIRONOLACTONE
MIDAZOLAM
MIDAZOLAM HYDROCHLORIDE
NORFLOXACIN
SERTRALINE
SERTRALINE HYDROCHLORIDE
SPIRONOLACTONE
   and mixtures thereof, wherein the patient receives an amount of the compound according to formula (I) of 20 mg.

In one embodiment, the CYP3A4 inhibitor of unspecified potency and/or a combination that comprises at least one CYP3A4 inhibitor of unspecified potency is selected from:
DESOGESTREL; ETHINYLESTRADIOL
FEXUPRAZAN HYDROCHLORIDE
HYDROCHLOROTHIAZIDE; SPIRONOLACTONE
MIDAZOLAM
MIDAZOLAM HYDROCHLORIDE
NORFLOXACIN
SERTRALINE
SERTRALINE HYDROCHLORIDE
SPIRONOLACTONE
SPIRONOLACTONE; TORASEMIDE
   and mixtures thereof, wherein the patient receives an amount of the compound according to formula (I) of 40 mg.

In one embodiment, the patient receives the compound according to formula (I) and at least one further medicament. In one embodiment, the patient receives the compound according to formula (I) and equal to or below 5 medications. In one embodiment, the patient receives the compound according to formula (I) and equal to or above 5 medications. In one embodiment, the patient receives the compound according to formula (I) and equal to or above 5 to equal to or below 9 medications. In one embodiment, the patient receives the compound according to formula (I) and equal to or above 5 medications. In one embodiment, the patient receives the compound according to formula (I) and equal to or below 9 medications. In one embodiment, the patient receives the compound according to formula (I) and equal to or above 5 medications. In one embodiment, the patient receives the compound according to formula (I) and equal to or above 9 medications. In one embodiment, the patient receives equal to or above 10 medications. Examples of co-medications are described above and below in detail. Examples of co-medications are loop diuretics, beta-blocker, ACE inhibitor (ACEi), angiotensin-receptor-blocker (ARB), Angiotensin Receptor-Neprilysin Inhibitor (ARNI), Calcium Channel Blockers, Sodium-glucose Cotransporter-2 Inhibitor (SGLT-2i), glucagon-like peptide-1 (GLP-1) agonists, CYP3A4 inducer, CYP3A4 inhibitor and/or mixtures thereof.

These multiple medications may be stated as: “non-polypharmacy”: <5 medications; “polypharmacy”: 5 to 9 medications; and “hyperpolypharmacy”: ≥10 medications.

Kansas City Cardiomyopathy Questionnaire (KCCQ) is a standardized patient reported outcomes (PRO) questionnaire which assesses symptoms, physical and social limitations, and quality of life in patients with heart failure (HF). Patients are asked to recall how their HF impacted their life over a 2-week recall period. The KCCQ is composed of different score domains including the Total symptom score (TSS), which depicts patients' perceptions on their symptoms related to heart failure (Green C P, Porter C B, Bresnahan D R, Spertus J A. Development and evaluation of the Kansas City Cardiomyopathy Questionnaire: a new health status measure for heart failure. J Am Coll Cardiol. 2000 April; 35 (5): 1245-55.)

In one embodiment, the patient has a KCCQ-TSS of equal to or above 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85,86, 87, 88.89. 90. 91, 92, 93, 94, 95, 96, 97, 98, 99, and 100. In one embodiment, the patient has a KCCQ-TSS of equal to or below 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85,86, 87, 88.89. 90. 91, 92, 93, 94, 95, 96, 97, 98, 99, and 100.

In one embodiment, the patient has a KCCQ-TSS of equal to or above 43 to equal to or below 91. In one embodiment, the patient has a KCCQ-TSS of equal to or above 43 to equal to or below 67. In one embodiment, the patient has a KCCQ-TSS of equal to or above 67 to equal to or below 91. In one embodiment, the patient has a KCCQ-TSS of 69+/−24. In one embodiment, the patient has a KCCQ-TSS of equal to or above 45 to equal to or below 93. In one embodiment, the patient has a KCCQ-TSS of qual or above 45 to equal to or below 69. In one embodiment, the patient has a KCCQ-TSS of equal to or above 69 to equal to or below 93. In one embodiment, the patient has a KCCQ-TSS of 66+/−24. In one embodiment, the patient has a KCCQ-TSS of equal to or above 42 to equal to or below 90. In one embodiment, the patient has a KCCQ-TSS of equal to or above 42 to equal to or below 66. In one embodiment, the patient has a KCCQ-TSS of equal to or above 66 to equal to or below 90. In one embodiment, the patient has a KCCQ-TSS of 68.7+/−23.1. In one embodiment, the patient has a KCCQ-TSS of 71.2+/−22.5. In one embodiment, the patient has a KCCQ-TSS of 66.0+/−24.5. In one embodiment, the patient has a KCCQ-TSS of 65.3+23.8. In one embodiment, the patient has a KCCQ-TSS of 68.5+/−23.7. In one embodiment, the patient has a KCCQ-TSS of 67.8+/−22.7. In one embodiment, the patient has a KCCQ-TSS of 62.3+/−25.4. In one embodiment, the patient has a KCCQ-TSS of 63.8+/−25.5.

Body mass index (BMI) is a value derived from the mass (weight) and height of a person. The BMI is defined as the body mass divided by the square of the body height, and is expressed in units of kg/m², resulting from mass in kilograms (kg) and height in metres (m).

In one embodiment, the patient has a body-mass-index (BMI) of equal to or above 15 kg/m².

In one embodiment, the patient has a body-mass-index (BMI) selected from of equal to or above 15, 16, 17, 18, 18.5, 19, 20, 21, 22, 23, 24, 24.5, 25, 26, 27, 28, 29, 30, 31, 32, 33, 34, 34.5, 35, 36, 37, 38, 39, 40, 41, 42, 43, 44, 45, 46, 47, 48, 49, and 50 kg/m². In one embodiment, the patient has a body-mass-index (BMI) selected from of equal to or below 15, 16, 17, 18, 18.5, 19, 20, 21, 22, 23, 24, 24.5, 25, 26, 27, 28, 29, 30, 31, 32, 33, 34, 34.5, 35, 36, 37, 38, 39, 40, 41, 42, 43, 44, 45, 46, 47, 48, 49, and 50 kg/m². In one embodiment, the patient has a body-mass-index (BMI) equal to or above 15 kg/m². In one embodiment, the patient has a body-mass-index (BMI) equal to or above 20 kg/m². In one embodiment, the patient has a body-mass-index (BMI) equal to or above 25 kg/m². In one embodiment, the patient has a body-mass-index (BMI) equal to or above 30 kg/m². In one embodiment, the patient has a body-mass-index (BMI) equal to or above 35 kg/m². In one embodiment, the patient has a body-mass-index (BMI) equal to or above 40 kg/m². In one embodiment, the patient has a body-mass-index (BMI) equal to or above 45 kg/m². In one embodiment, the patient has a body-mass-index (BMI) equal to or above 50 kg/m². In one embodiment, the patient has a body-mass-index (BMI) equal to or below 15 kg/m². In one embodiment, the patient has a body-mass-index (BMI) equal to or below 20 kg/m². In one embodiment, the patient has a body-mass-index (BMI) equal to or below 25 kg/m². In one embodiment, the patient has a body-mass-index (BMI) equal to or below 30 kg/m². In one embodiment, the patient has a body-mass-index (BMI) equal to or below 35 kg/m². In one embodiment, the patient has a body-mass-index (BMI) equal to or below 40 kg/m². In one embodiment, the patient has a body-mass-index (BMI) equal to or below 45 kg/m². In one embodiment, the patient has a body-mass-index (BMI) equal to or below 50 kg/m².

In one embodiment, the patient has a body-mass-index (BMI) of 30+/−6 kg/m². In one embodiment, the patient has a body-mass-index (BMI) of equal to or above 24 to equal to or below 36 kg/m². In one embodiment, the patient has a body-mass-index (BMI) of equal to or above 24 to equal to or below 30 kg/m². In one embodiment, the patient has a body-mass-index (BMI) of equal to or above 30 to equal to or below 36 kg/m². In one embodiment, the patient has a body-mass-index (BMI) of equal to or above 30 kg/m². In one embodiment, the patient has a body-mass-index (BMI) of equal to or above 15 to equal to or below 18.4 kg/m². In one embodiment, the patient has a body-mass-index (BMI) of equal to or above 18.5 to equal to or below 24.9 kg/m². In one embodiment, the patient has a body-mass-index (BMI) of equal to or above 25 to equal to or below 29.9 kg/m². In one embodiment, the patient has a body-mass-index (BMI) of equal to or above 30 to equal to or below 34.9 kg/m². In one embodiment, the patient has a body-mass-index (BMI) of equal to or above 35 to equal to or below 39.9 kg/m². In one embodiment, the patient has a body-mass-index (BMI) of equal to or above 40 to equal to or below 50 kg/m².

In one embodiment, the patient was hospitalized for heart failure (HHF) and has at least one of
KCCQ-TSS: 53±24, or 70±23, or 71±22
eGFR (mL/min/1.73 m²): 60±20, or 63=20, or 62±19
NT-proBNP (ng/L) (median): 1790, or 1691, or 1322
History of Atrial Flutter
History of Atrial fibrillation,
History of Diabetes.
History of coronary artery disease (CAD)
History of chronic kidney disease (CKD)
History of stroke
History of transient ischemic attack (TIA)
History of chronic obstructive pulmonary disease (COPD)
History of metabolic syndrome
History of frailty
History of peripheral artery disease (PAD), and/or
History of obstructive sleep apnea (OSA)

In one embodiment, the patient is selected from one or more of the following subgroups:
Potassium subgroups are specified above according to the following cutpoints (≤4.5, >4.5 mmol/L). Additional subgroups can be defined by median, quantiles, and other standard definitions of hypo-, hyper- and normokalemia.
Sodium subgroups defined by the median, quantiles, and standard definitions of hypo-, hyper- and normonatremia.
Chloride subgroups defined by the median, quantiles, and standard definitions of hypo-, hyper-and normo-chloremia.
Hemoglobin and hematocrit subgroups defined by the median, quantiles, and standard definitions of anemia.
eGFR subgroups can be specified above according to the following cutpoints <60, ≥60 mL/min/1.73 m².
Focused examination of Stage 4 CKD (if eGFR was less than 30 mL/min/1.73 m²)
UACR subgroups are specified above according to the following cutpoints (<30 vs. ≥30 mg/g).
BUN and BUN/creatinine ratio subgroups defined by the median and quantiles.
Patients suspected to have primary hyperaldosteronism based on renin and aldosterone levels
BMI subgroups are specified above according to the following cutpoints (<30 kg/m²≥30 kg/m²)
Subgroups defined by standard nutritional indices e.g., the geriatric nutritional risk index (GNRI), prognostic nutritional index (PNI), and controlling nutritional status (CONUT), examining these as categorical variables using recognized cut points, medians, quantiles, and as continuous variables.
Age subgroups are specified above according to the following cutpoints (median age).
Systolic blood pressure subgroups are specified above according to the following cutpoints (median age). SBP and DBP can be assessed at the median, quantiles, and various global definitions of hypertension (based on both SBP and DBP).
Patients with apparent treatment resistant hypertension
Atrial fibrillation
Atrial fibrillation or atrial flutter on baseline ECG
a previous history of atrial fibrillation/flutter, optionally together with the overlap between medical history and baseline ECG and derived history of persistent/permanent AF versus paroxysmal AF.
Patients with improved/recovered LVEF (those who had LVEF ≤40% at any time prior to randomization)
LVEF at the following cutpoint: <60%, ≥60%, ≤49%, 50 to 59%, ≥60%—to examine those with HF with mildly reduced ejection fraction.
two-way interaction between sex and LVEF will be examined.
Other anthropometric indices e.g., waist-to-hip ratio, waist-to-height ratio using quantiles and recognized cutpoints
Time from prior HF hospitalization
Time from index HF diagnosis
Patients with COPD
Patients with OSA
Patients with cirrhosis
Patients with any atherosclerotic cardiovascular disease.
Patients with history of stroke/TIA.
Patients with history of coronary artery disease/prior MI
Patients with history of coronary artery bypass graft surgery
Patients with peripheral artery disease
Patients with metabolic syndrome (using standard definitions)
Subgroups based on baseline use and dosing of diuretics
Patients with multimorbidity and frailty
Patients with cardio-renal-metabolic overlap (intersection of atherosclerotic cardiovascular disease, CKD, and diabetes)
Total baseline medications will be categorized as (“non-polypharmacy”: <5 medications; “polypharmacy”: 5 to 9 medications; and “hyperpolypharmacy”: ≥10 medications)
Number of blood pressure lowering therapies at baseline
Patients with baseline risk as determined by the Meta-Analysis Global Group in Chronic (MAGGIC) Heart Failure Risk Score and other HF risk scores
Patients with baseline risk as determined by the Kidney Failure Risk Equation
Subgroups based on baseline evidence of congestion and congestion scores
Regional subgroups based on socioeconomic differences based on the GINI coefficient
Subgroups based on KCCQ-TSS and other KCCQ domains at baseline.
Subgroups based on EQ-5D-5L VAS and index scores at baseline.
Time from diagnosis of heart failure, analyzed as both a continuous and a categorical variable
Time from HF hospitalization continuously and focused examination for those never hospitalized
NT-proBNP and high-sensitivity troponin will be examined at median, quantiles, and as continuous measures
History of any prior MRA use prior to randomization.
Background HF therapies including focused examination of patients on various combinations of therapies (including the Heart Failure Collaboratory score)
Patients on or off betablockers
Patients on or off potassium supplements
Patients on or off potassium lowering therapies
Patients with phenotypic characteristics of amyloid cardiomyopathy, based on validated clinical models/scores

In the following, embodiments of the dose, dosing scheme, titration, up-titration and/or down-titration of the compound according to formula (I) are described. These embodiments also apply to embodiments, in which a hydrate, solvate, pharmaceutically acceptable salt thereof, a polymorph thereof or polymorph (I) is used.

The term “dose” is used as typically used in the art. The term “dose” is usually applied to the quantity of a drug or other agent administered for therapeutic purposes to the body. The term “dose” and the term “the amount the patient receives” can be used synonymously. In one example, a dose of 10 mg of the compound according to formula (I) is administered to the patient. In other words: the patient receives an amount of 10 mg of the compound of formula (I).

The term “dose” can include a quantity of the drug that the patient takes at the same time. The term “dose” can include a quantity of the drug that the patient takes consecutively. The respective amount of the drug can be taken immediately one after the other or they can be taken at a time interval. This interval can be the same or different.

The “dose” or the “amount the patient receives” can also refer to a single dose, a daily dose or multiple daily doses. For example, if the patient receives 10 mg of the compound according to formula (I) twice a day, this is a single dose of 10 mg, but a daily dose of 20 mg. The term “dose”/“the amount the patient receives” can, thus, refer to the single dose (10 mg) or the total daily dose (20 mg).

A dose can be a once daily, twice daily, four times daily or multiple daily dose.

The amount or dose can be delivered to the patient, for example, using one pharmaceutical composition or one or more pharmaceutical compositions. Pharmaceutical compositions are described in detail below.

For example, a dose of 40 mg of the compound according to formula (I) can be administered to the patient using four tablets comprising 10 mg each. These four tablets can be taken at the same time or consecutively. In another example, a dose of 40 mg of the compound according to formula (I) can be administered to the patient using two tablets comprising 20 mg each. For example, a dose of 40 mg of the compound according to formula (I) can be administered to the patient using two tablets comprising 10 mg each and one tablet comprising 20 mg.

In one embodiment, the compound according to formula (I) is used in an amount of 0.25 mg to 80 mg. In one embodiment, the compound according to formula (I) is used in an amount of 0.25 mg to 40 mg. In one embodiment, the compound according to formula (I) is used in an amount of 0.25 mg to 20 mg. In one embodiment, the compound according to formula (I) is used in an amount of 0.25 mg to 10 mg. In one embodiment, the compound according to formula (I) is used in an amount of 0.25 mg to 5 mg. In one embodiment, the compound according to formula (I) is used in an amount of 5 to 80 mg. In one embodiment, the compound according to formula (I) is used in an amount of 5 to 80 mg, 5 to 70 mg, 5 to 60 mg, 5 to 50 mg, 5 to 40 mg, 10 to 80 mg, 10 to 70 mg, 10 to 60 mg, 10 to 50 mg or 10 to 40 mg. In one embodiment, the compound according to formula (I) is used in an amount of 10 to 40 mg. In one embodiment, the compound according to formula (I) is used in an amount of 20 to 40 mg. In one embodiment, the compound according to formula (I) is used in an amount of 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 55, 60, 65, 70, 75 or 80 mg. In one embodiment, the compound according to formula (I) is used in an amount of 5, 10, 15, 20, 25, 30, 35, 40, 45, 50, 55, 60, 65, 70, 75 or 80 mg. In one embodiment, the compound according to formula (I) is used in an amount of 5 mg. In one embodiment, the compound according to formula (I) is used in an amount of 10 mg. In one embodiment, the compound according to formula (I) is used in an amount of 15 mg. In one embodiment, the compound according to formula (I) is used in an amount of 20 mg. In one embodiment, the compound according to formula (I) is used in an amount of 25 mg. In one embodiment, the compound according to formula (I) is used in an amount of 30 mg. In one embodiment, the compound according to formula (I) is used in an amount of 35 mg. In one embodiment, the compound according to formula (I) is used in an amount of 40 mg. In one embodiment, the compound according to formula (I) is used in an amount of 5 mg, 10 mg, 15 mg, 20 mg, 25 mg, 30 mg, 35 mg or 40 mg. In one embodiment, the compound according to formula (I) is used in an amount of 5 mg, 10 mg, 20 mg, 30 mg or 40 mg.

The amount of the compound according to formula (I) can be adjusted. In one embodiment thereof, the dose is adjusted by up-titration. In one embodiment thereof, the dose is adjusted by down-titration. In one embodiment thereof, the dose is continued. In one embodiment thereof, the treatment is interrupted.

In one embodiment the treatment or prevention is interrupted due to hyperkalemia, renal impairment, diarrhoea, nausea, asthenia, dyspnoea, hypotension, sepsis, septic shock, COVID-19, Cardiac arrest, COVID-19 pneumonia, Cardiac failure, Respiratory failure, Lung neoplasm malignant, Pneumonia, Atrial fibrillation, COVID-19, Acute kidney injury, Angina unstable, Anaemia, COVID-19 pneumonia, Urinary tract infection, Syncope, Chest pain, Chronic obstructive pulmonary disease, Angina pectoris, Cellulitis, Sepsis, Coronary artery disease, Cardiac failure, Sudden death, Femur fracture, Fall, Gastrointestinal haemorrhage, hypokalemia and/or Hyperkalemia.

In one embodiment the dose is adjusted based on one or more of the factors selected from LVEF, NYAH, eGFR, UACR, Potassium level, sodium level, chloride level, urea level, blood urea nitrogen (BUN), high-sensitivity Troponin T level, natriuretic peptide (NT-proBNP), blood pressure (BP), systolic blood pressure, average heart rate (resting), previous diseases, prior MRA use, heart rate (or pulse rate), comedication, KCCQ-TSS, Body mass index (BMI), comedication, CYP3A4 inhibitor, CYP3A4 inducer, loop diuretics, beta-blocker, ACE inhibitor (ACEi), angiotensin-receptor-blocker (ARB), Angiotensin Receptor-Neprilysin Inhibitor (ARNI), Calcium Channel Blockers, Sodium-glucose Cotransporter-2 Inhibitor (SGLT-2i), and mixtures thereof. In one embodiment thereof, the dose is adjusted by up-titration. In one embodiment thereof, the dose is adjusted by down-titration. In one embodiment thereof, the dose is continued (or maintained). In one embodiment thereof, the treatment is interrupted.

The dose can be adjusted based on the eGFR.

In one embodiment thereof, the dose is adjusted by up-titration. In one embodiment thereof, the dose is adjusted by down-titration. In one embodiment thereof, the dose is continued. In one embodiment thereof, the treatment is interrupted.

In one embodiment, the patient receives the compound according to formula (I) in an amount of 10 mg, if the patient has an eGFR of equal to or below 60 mL/min/1.73 m². In one embodiment, the patient receives the compound according to formula (I) starting dose in an amount of 10 mg, if the patient has an eGFR of equal to or below 60 mL/min/1.73 m².

In one embodiment, the patient receives the compound according to formula (I) in an amount of 20 mg, if the patient has an eGFR of equal to or above 60 mL/min/1.73 m². In one embodiment, the patient receives the compound according to formula (I) starting dose in an amount of 20 mg, if the patient has an eGFR of equal to or above 60 mL/min/1.73 m².

In one embodiment, the patient receives the compound according to formula (I) in an amount of 40 mg, if the patient has an eGFR of equal to or above 60 mL/min/1.73 m². In one embodiment, the patient receives the compound according to formula (I) as starting dose in an amount of 40 mg, if the patient has an eGFR of equal to or above 60 mL/min/1.73 m².

In one embodiment, the patient receives a lower dose of the compound according to formula (I), and patient has an eGFR of equal to or below 60 mL/min/1.73 m², then the dose is up-titrated to the next higher dose. In one embodiment, the patient receives a higher dose of the compound according to formula (I), and patient has an eGFR of equal to or above 60 mL/min/1.73 m², then the dose is down-titrated to the next lower dose. Further examples for “lower dose” are described below. The term “lower dose” and “next lower dose”, “down-titrated dose” and “down-titrated daily dose” are interchangeable and can be used synonymously. Accordingly, the term “higher dose” and “next higher dose” is synonymous with “up-titrated dose” or “up-titrated daily dose”.

In one embodiment, “next higher dose” or “next lower dose” refer to the dose that is immediately higher or lower, respectively, compared to a previously mentioned dose within a previously defined dose range. This range may include for example the following doses: 2.5, 5, 10, 15, 20, 25, 30, 35, 40, 45, 50, 55, 60, 65, 70, 75 or 80 mg. For example, if 75 mg is the current, first or previously mentioned dose, then 80 mg is considered the next higher dose. Conversely, if 80 mg is the current, first or previously mentioned dose, then 75 mg is considered the next lower dose.

In one embodiment, “next higher dose” or “next lower dose” refers to a dosage that is administered as the third step in a treatment sequence after a patient has already received a first and second dose for the treatment of a disease as previously outlined. For instance, if a patient is treated with the compound of formula (I), starting with a 40 mg dose in the first step, the “next higher dose” scenario would involve administering 60 mg in the second step, followed by an increase to 80 mg in the third step, marking it as the next higher dose relative to the initial 40 mg. Conversely, in the “next lower dose” scenario, after the initial 40 mg dose, the patient would receive 20 mg in the second step, and then the dosage would be further reduced to 10 mg in the third step, establishing it as the next lower dose in relation to the starting dose.

In one embodiment, the dose the patient is receiving is up-titrated.

In one embodiment, the dose the patient is receiving is up-titrated depending on the potassium level.

In one embodiment, the dose the patient is receiving is up-titrated, if the potassium level is equal to or below 3, 3.5, 4, 4.5, 4.8, 5, or 5.5 mmol/L. In one embodiment, the dose the patient is receiving is up-titrated, if the potassium level is equal to or below 3, 3.5, 4, 4.5, 4.7, 4.8, 4.9, 5, 5.1, 5.4, 5.5, or 5.6 mmol/L. In one embodiment, the dose the patient is receiving is up-titrated, if the potassium level is equal to or above 3, 3.5, 4, 4.5, 4.8, 5 or 5.5 mmol/L. In one embodiment, the dose the patient is receiving is up-titrated, if the potassium level is equal to or above 3, 3.5, 4, 4.5, 4.8, or 5 or 5.5 mmol/L, but below 6 mmol/L.

In one embodiment, the dose the patient is receiving is up-titrated, if the potassium level is equal to or below 4.0 mmol/L. In one embodiment, the dose the patient is receiving is up-titrated, if the potassium level is equal to or below 4.5 mmol/L. In one embodiment, the dose the patient is receiving is up-titrated, if the potassium level is equal to or below 4.8 mmol/L. In one embodiment, the dose the patient is receiving is up-titrated, if the potassium level is equal to or below 5 mmol/L. In one embodiment, the dose the patient is receiving is up-titrated, if the potassium level is equal to or below 5.5 mmol/L.

In one embodiment, the dose the patient is receiving is up-titrated, if the potassium level is equal to or above 4.5 mmol/L to equal to or below to below 5.5 mmol/L. In one embodiment, the dose the patient is receiving is up-titrated, if the potassium level is equal to or above 4.8 mmol/L to below 5.5 mmol/L. In one embodiment, the dose the patient is receiving is up-titrated, if the potassium level is equal to or above 5 mmol/L to below 5.5 mmol/L. In one embodiment, the dose the patient is receiving is up-titrated, if the potassium level is equal to or above 4.5 mmol/L to equal to or below to equal to or below 5 mmol/L. In one embodiment, the dose the patient is receiving is up-titrated, if the potassium level is equal to or above 4.8 mmol/L to equal to or equal to or below 5 mmol/L.

In one embodiment, the dose the patient is receiving is maintained of the potassium level is equal to or above 5 to below 5.5 mmol/L. A synonym for “maintained” is for example “continued”.

In one embodiment, the dose the patient is receiving is continued.

In one embodiment, the dose the patient is receiving is continued is depending on the potassium level.

In one embodiment, the dose the patient is receiving is continued, if the potassium level is equal to or below 3, 3.5, 4, 4.5, 4.8, 5 or 5.5 mmol/L. In one embodiment, the dose the patient is receiving is continued, if the potassium level is equal to or above 3, 3.5, 4, 4.5, 4.8, 5 or 5.5 mmol/L, but below 6 mmol/L. In one embodiment, the dose the patient is receiving is continued, if the potassium level is equal to or above 3, 3.5, 4, 4.5, 4.8, or 5 or 5.5 mmol/L, but below 6 mmol/L. The dose the patient is receiving can be selected from 10 mg, 20 mg and 40 mg. The dose the patient is receiving can be 10 mg. The dose the patient is receiving can be 20 mg. The dose the patient is receiving can be 40 mg.

In one embodiment, the dose the patient is receiving is continued, if the potassium level is equal to or below 4.5, 4.8, 5 or 5.5 mmol/L. In one embodiment, the dose the patient is receiving is continued, if the potassium level is equal to or above 4.5, 4.8, 5 or 5.5 mmol/L. In one embodiment, the dose the patient is receiving is continued, if the potassium level is equal to or above 4.5, 4.8, or 5 or 5.5 mmol/L, but below 6 mmol/L. The dose the patient is receiving can be selected from 10 mg, 20 mg and 40 mg. The dose the patient is receiving can be 10 mg. The dose the patient is receiving can be 20 mg. The dose the patient is receiving can be 40 mg.

In one embodiment, the dose the patient is receiving is continued, if the potassium level is equal to or below 4.8, 5 or 5.5 mmol/L. In one embodiment, the dose the patient is receiving is continued, if the potassium level is equal to or above 4.8, 5 or 5.5 mmol/L. In one embodiment, the dose the patient is receiving is continued, if the potassium level is equal to or above 4.8, or 5 or 5.5 mmol/L, but below 6 mmol/L. The dose the patient is receiving can be selected from 10 mg, 20 mg and 40 mg. The dose the patient is receiving can be 10 mg. The dose the patient is receiving can be 20 mg. The dose the patient is receiving can be 40 mg.

In one embodiment, the dose the patient is receiving is continued, if the potassium level is equal to or below 4.8 mmol/L. In one embodiment, the dose the patient is receiving is continued, if the potassium level is equal to or above 4.8 mmol/L. In one embodiment, the dose the patient is receiving is continued, if the potassium level is equal to or above 4.8 mmol/L, but below 5.5 mmol/L. The dose the patient is receiving can be selected from 10 mg, 20 mg and 40 mg. The dose the patient is receiving can be 10 mg. The dose the patient is receiving can be 20 mg. The dose the patient is receiving can be 40 mg.

In one embodiment, the dose the patient is receiving is continued, if the potassium level is equal to or below 5 mmol/L. In one embodiment, the dose the patient is receiving is continued, if the potassium level is equal to or above 5 mmol/L. In one embodiment, the dose the patient is receiving is continued, if the potassium level is equal to or above 5 mmol/L, but below 6 mmol/L. The dose the patient is receiving can be selected from 10 mg, 20 mg and 40 mg. The dose the patient is receiving can be 10 mg. The dose the patient is receiving can be 20 mg. The dose the patient is receiving can be 40 mg.

In one embodiment, the dose the patient is receiving is continued, if the potassium level is equal to or below 5.5 mmol/L. In one embodiment, the dose the patient is receiving is continued, if the potassium level is equal to or above 5.5 mmol/L, but below 6 mmol/L. The dose the patient is receiving can be selected from 10 mg, 20 mg and 40 mg. The dose the patient is receiving can be 10 mg. The dose the patient is receiving can be 20 mg. The dose the patient is receiving can be 40 mg.

In one embodiment, the patient the treatment of the patient is interrupted. In one embodiment, the patient the treatment of the patient is interrupted, if the potassium level is equal to or above 5, 5.5 or 6 mmol/L. In one embodiment, the patient the treatment of the patient is interrupted, if the potassium level is equal to or above 5.5 or 6 mmol/L. In one embodiment, the patient the treatment of the patient is interrupted, if the potassium level is equal to or above 5 mmol/L. In one embodiment, the patient the treatment of the patient is interrupted, if the potassium level is equal to or above 5.5 mmol/L. In one embodiment, the patient the treatment of the patient is interrupted, if the potassium level is equal to or above 6 mmol/L. In one embodiment, the patient the treatment of the patient is interrupted, if the potassium level is equal to or above 6.5 mmol/L.

In one embodiment, the patient the treatment of the patient is interrupted, if the potassium level is between 5 to 6.5 mmol/L. In one embodiment, the patient the treatment of the patient is interrupted, if the potassium level is between 5 to 6 mmol/L. In one embodiment, the patient the treatment of the patient is interrupted, if the potassium level is between 5.5 to 6.5 mmol/L. In one embodiment, the patient the treatment of the patient is interrupted, if the potassium level is between 5.5 to 6 mmol/L.

In one embodiment, the treatment is interrupted, if
the patient receives a dose selected from 10, 20, 30 and 40 mg; and
the potassium level is equal to or above 5, 5.5 or 6 mmol/L.

In one embodiment, the treatment is interrupted, if
the patient receives a dose selected from 10, 20, 30 and 40 mg; and
the potassium level is equal to or above 5, 5.5, 5.9, 6, or 6.1 mmol/L.

In one embodiment, the treatment is interrupted, if
the patient receives a dose selected from 10, 20, 30 and 40 mg; and
the potassium level is equal to or above 5.5 or 6 mmol/L.

In one embodiment, the treatment is interrupted, if
the patient receives a dose selected from 10, 20, 30 and 40 mg; and
the potassium level is equal to or above 5.5 mmol/L.

In one embodiment, the treatment is interrupted, if
the patient receives a dose selected from 10, 20, 30 and 40 mg; and
the potassium level is equal to or above 6 mmol/L.

In one embodiment, the treatment is interrupted, if
the patient receives a dose selected from 10, 20, 30 and 40 mg; and
the potassium level is between equal to or above 5 to 6.5 mmol/L.

In one embodiment, the treatment is interrupted, if
the patient receives a dose selected from 10, 20, 30 and 40 mg; and
the potassium level is between equal to or above 5.5 to 6 mmol/L.

In one embodiment, the treatment is interrupted, if
the patient receives a dose of 10 mg; and
the potassium level is equal to or above 5, 5.5 or 6 mmol/L.

In one embodiment, the treatment is interrupted, if
the patient receives a dose of 20 mg; and
the potassium level is equal to or above 5, 5.5 or 6 mmol/L.

In one embodiment, the treatment is interrupted, if
the patient receives a dose of 30 mg; and
the potassium level is equal to or above 5, 5.5 or 6 mmol/L.

In one embodiment, the treatment is interrupted, if
the patient receives a dose of 40 mg; and
the potassium level is equal to or above 5, 5.5 or 6 mmol/L.

In one embodiment, the treatment is interrupted, if
the patient receives a dose selected from 10 mg; and
the potassium level is equal to or above 5.5 mmol/L.

In one embodiment, the treatment is interrupted, if
the patient receives a dose selected from 10 mg; and
the potassium level is equal to or above 6 mmol/L.

In one embodiment, the treatment is interrupted, if
the patient receives a dose selected from 10 mg; and
the potassium level is between equal to or above 5 to 6.5 mmol/L.

In one embodiment, the treatment is interrupted, if
the patient receives a dose selected from 10 mg; and
the potassium level is between equal to or above 5.5 to 6 mmol/L.

In one embodiment, the treatment is interrupted, if
the patient receives a dose selected from 20 mg; and
the potassium level is equal to or above 5.5 mmol/L.

In one embodiment, the treatment is interrupted, if
the patient receives a dose selected from 20 mg; and
the potassium level is equal to or above 6 mmol/L.

In one embodiment, the treatment is interrupted, if
the patient receives a dose selected from 20 mg; and
the potassium level is between equal to or above 5 to 6.5 mmol/L.

In one embodiment, the treatment is interrupted, if
the patient receives a dose selected from 20 mg; and
the potassium level is between equal to or above 5.5 to 6 mmol/L.

In one embodiment, the treatment is interrupted, if
the patient receives a dose selected from 30 mg; and
the potassium level is equal to or above 5.5 mmol/L.

In one embodiment, the treatment is interrupted, if
the patient receives a dose selected from 30 mg; and
the potassium level is equal to or above 6 mmol/L.

In one embodiment, the treatment is interrupted, if
the patient receives a dose selected from 30 mg; and
the potassium level is between equal to or above 5 to 6.5 mmol/L.

In one embodiment, the treatment is interrupted, if
the patient receives a dose selected from 30 mg; and
the potassium level is between equal to or above 5.5 to 6 mmol/L.

In one embodiment, the treatment is interrupted, if
the patient receives a dose selected from 40 mg; and
the potassium level is equal to or above 5.5 mmol/L.

In one embodiment, the treatment is interrupted, if
the patient receives a dose selected from 40 mg; and
the potassium level is equal to or above 6 mmol/L.

In one embodiment, the treatment is interrupted, if
the patient receives a dose selected from 40 mg; and
the potassium level is between equal to or above 5 to 6.5 mmol/L.

In one embodiment, the treatment is interrupted, if
the patient receives a dose selected from 40 mg; and
the potassium level is between equal to or above 5.5 to 6 mmol/L.

The treatment of the patient can be interrupted and then restarted. In one embodiment, the treatment of the patient is restarted.

In one embodiment, the dose with which the treatment is restarted is selected from 10 mg, 20 mg and 40 mg. In one embodiment, the dose with which the treatment is restarted is 10 mg. In one embodiment, the dose with which the treatment is restarted is 20 mg. In one embodiment, the dose with which the treatment is restarted is 40 mg.

In one embodiment, the treatment of the patient is restarted, if the potassium level is equal to or below 3, 3.5, 4, 4.5, 4.8, 5 or 5.5 mmol/L. In one embodiment, the treatment of the patient is restarted, if the potassium level is equal to or above 3, 3.5, 4, 4.5, 4.8, 5 or 5.5 mmol/L. In one embodiment, the treatment of the patient is restarted, if the potassium level is equal to or above 3, 3.5, 4, 4.5, 4.8, or 5 or 5.5 mmol/L, but below 5.5 mmol/L. The dose with which the treatment is restarted can be selected from 10 mg, 20 mg and 40 mg. The dose with which the treatment is restarted can be 10 mg. The dose with which the treatment is restarted can be 20 mg. The dose with which the treatment is restarted can be 40 mg.

In one embodiment, the treatment of the patient is restarted, if the potassium level is equal to or below 4.5, 4.8, 5 or 5.5 mmol/L. In one embodiment, the treatment of the patient is restarted, if the potassium level is equal to or above 4.5, 4.8, 5 or 5.5 mmol/L. In one embodiment, the treatment of the patient is restarted, if the potassium level is equal to or above 4.5, 4.8, or 5 or 5.5 mmol/L, but below 5.5 mmol/L. The dose with which the treatment is restarted can be selected from 10 mg, 20 mg and 40 mg. The dose with which the treatment is restarted can be 10 mg. The dose with which the treatment is restarted can be 20 mg. The dose with which the treatment is restarted can be 40 mg.

In one embodiment, the treatment of the patient is restarted, if the potassium level is equal to or below 4.8, 5 or 5.5 mmol/L. In one embodiment, the treatment of the patient is restarted, if the potassium level is equal to or above 4.8, 5 or 5.5 mmol/L. In one embodiment, the treatment of the patient is restarted, if the potassium level is equal to or above 4.8, or 5 or 5.5 mmol/L, but below 5.5 mmol/L. The dose with which the treatment is restarted can be selected from 10 mg, 20 mg and 40 mg. The dose with which the treatment is restarted can be 10 mg. The dose with which the treatment is restarted can be 20 mg. The dose with which the treatment is restarted can be 40 mg.

In one embodiment, the treatment of the patient is restarted, if the potassium level is equal to or below 4.8 mmol/L. In one embodiment, the treatment of the patient is restarted, if the potassium level is equal to or above 4.8 mmol/L. In one embodiment, the treatment of the patient is restarted, if the potassium level is equal to or above 4.8 mmol/L, but below 5.5 mmol/L. The dose with which the treatment is restarted can be selected from 10 mg, 20 mg and 40 mg. The dose with which the treatment is restarted can be 10 mg. The dose with which the treatment is restarted can be 20 mg. The dose with which the treatment is restarted can be 40 mg.

In one embodiment, the treatment of the patient is restarted, if the potassium level is equal to or below 5 mmol/L. In one embodiment, the treatment of the patient is restarted, if the potassium level is equal to or above 5 mmol/L. In one embodiment, the treatment of the patient is restarted, if the potassium level is equal to or above 5 mmol/L, but below 5.5 mmol/L. The dose with which the treatment is restarted can be selected from 10 mg, 20 mg and 40 mg. The dose with which the treatment is restarted can be 10 mg. The dose with which the treatment is restarted can be 20 mg. The dose with which the treatment is restarted can be 40 mg.

In one embodiment, the treatment of the patient is restarted, if the potassium level is equal to or below 5.5 mmol/L. In one embodiment, the treatment of the patient is restarted, if the potassium level is equal to or above 5.5 mmol/L. The dose with which the treatment is restarted can be selected from 10 mg, 20 mg and 40 mg. The dose with which the treatment is restarted can be 10 mg. The dose with which the treatment is restarted can be 20 mg. The dose with which the treatment is restarted can be 40 mg. In one embodiment, the treatment is restarted, wherein
the patient receives a dose selected from 10, 20, 30, and 40 mg;
the potassium level is equal to or below 4.5, 4.8, 5, 5.5, 6 or 6.5 mmol/L.

In one embodiment, the treatment is restarted, wherein
the patient receives a dose selected from 10, 20, 30, and 40 mg;
the potassium level is equal to or below 5 mmol/L.

In one embodiment, the treatment is restarted, wherein
the patient receives a dose selected from 10, 20, 30, and 40 mg;
the potassium level is equal to or below 5.5 mmol/L.

In one embodiment, the treatment is restarted, wherein
the patient receives a dose selected from 10, 20, 30, and 40 mg;
the potassium level is equal to or below 6 mmol/L.

In one embodiment, the treatment is restarted, wherein
the patient receives a dose of 10 mg;
the potassium level is equal to or below 5 mmol/L.

In one embodiment, the treatment is restarted, wherein
the patient receives a dose of 20 mg;
the potassium level is equal to or below 5 mmol/L.

In one embodiment, the treatment is restarted, wherein
the patient receives a dose of 30 mg;
the potassium level is equal to or below 5 mmol/L.

In one embodiment, the treatment is restarted, wherein
the patient receives a dose of 40 mg;
the potassium level is equal to or below 5 mmol/L.

In one embodiment, the treatment is restarted, wherein
the patient receives a dose of 10 mg;
the potassium level is equal to or below 5.5 mmol/L.

In one embodiment, the treatment is restarted, wherein
the patient receives a dose of 20 mg;
the potassium level is equal to or below 5.5 mmol/L.

In one embodiment, the treatment is restarted, wherein
the patient receives a dose of 30 mg;
the potassium level is equal to or below 5.5 mmol/L.

In one embodiment, the treatment is restarted, wherein
the patient receives a dose of 40 mg;
the potassium level is equal to or below 5.5 mmol/L.

Further consideration might apply to the dose adjustment:

In case the participant receives the lowest dose, no further decrease might be possible after interruption. The dose level can be 10, 20, 30 or 40 mg. The treatment can be restarted at the same dose level once potassium level is equal to or below 4.5, 4.8, 5, 5.5, 6 or 6.5 mmol/L. The treatment can be restarted at the same dose level once potassium level is equal to or below 4.5, 4.8, 5, or 5.5 mmol/L. The treatment can be restarted at the same dose level once potassium level is equal to or below 5.5 mmol/L. Serum/plasma potassium can be measured at a safety visit after re-starting treatment or dose adjustment. Serum/plasma potassium is to be measured at a safety visit 4 weeks±7 days after re-starting treatment or dose adjustment.

In case the participant receives the lowest dose, but hyperkalemia recurs soon after a previous event of hyperkalemia leading to interruption of study intervention, and there is no explanation for the recurring hyperkalemia event other than intake of the dose, the treatment can be prematurely and/or permanently discontinued.

In the following, dosing schemes are described. In some embodiment, for the dosing schemes for up-titration and/or down-titration described herein the following embodiments can apply:

In some embodiments, the amount of the compound according to formula (I) is adjusted by up-titration. Here, the patient receives a lower dose, which is up-titrated to a higher dose. The patient can receive a lower dose, which is up-titrated to the next higher dose. In some embodiments, the amount of the compound according to formula (I) is adjusted by down-titration. Here, the patient receives a higher dose, which is down-titrated to a lower dose. The patient can receive a higher dose, which is down-titrated to the next lower dose.

In one embodiment, the “lower dose” of the compound according to formula (I) is between 0.5 to 60 mg. In one embodiment, the “lower dose” of the compound according to formula (I) is between 2.5 to 60 mg. In one embodiment, the “lower dose” of the compound according to formula (I) is selected from 2.5 mg, 5 mg, 10 mg, 20 mg, 30 mg, 40 mg, and 60 mg. In one embodiment, the “lower dose” is selected from 5 mg, 10 mg, 20 mg, and 30 mg. In one embodiment, the “lower dose” is 5 mg. In one embodiment, the “lower dose” is 10 mg. In one embodiment, the “lower dose” is 20 mg. In one embodiment, the “lower dose” is 30 mg.

The “next lower dose” can be a dose that is lower than the dose the patient is currently receiving. For example, if the currently receiving or the higher dose is 40 mg, the next lower dose can be for example 30 mg, 20 mg, 10 mg, or 5 mg. In one example the higher dose is 40 mg and the next lower dose is 30 mg. In one example the higher dose is 40 mg and the next lower dose is 20 mg. In one example the higher dose is 40 mg and the next lower dose is 10 mg. In one example the higher dose is 40 mg and the next lower dose is 5 mg.

In one embodiment, “lower dose” and “next lower dose” are synonyms.

In one embodiment, the “next lower dose” is selected from 5 mg, 10 mg, 20 mg, 30 mg, 40 mg, 60 mg, and 80 mg. In one embodiment, the “next lower dose” is selected from 10 mg, 20 mg, and 30 mg. In one embodiment, the “next lower dose” is 5 mg. In one embodiment, the “next lower dose” is 10 mg. In one embodiment, the “next lower dose” is 20 mg. In one embodiment, the “next lower dose” is 30 mg. In one embodiment, the “next lower dose” is 40 mg. In one embodiment, the “next lower dose” is 60 mg.

In one embodiment, the “higher dose” of the compound according to formula (I) is between 0.5 to 80 mg. In one embodiment, the “higher dose” of the compound according to formula (I) is between 2.5 to 80 mg. In one embodiment, the “higher dose” of the compound according to formula (I) is selected from 5 mg, 10 mg, 20 mg, 30 mg, 40 mg, 60 mg, and 80 mg. In one embodiment, the “higher dose” is selected from 20 mg, 30 mg, and 40 mg. In one embodiment, the “higher dose” is 20 mg. In one embodiment, the “higher dose” is 30 mg. In one embodiment, the “higher dose” is 40 mg.

The “next higher dose” can be a dose that is higher than the dose the patient is currently receiving. For example, if the currently receiving or the lower dose is 10 mg, the next higher dose can be for example 20 mg, 30 mg or 40 mg. In one example the lower dose 10 mg and the next higher dose is 20 mg. In one example the lower dose 10 mg and the next higher dose is 30 mg. In one example the lower dose 20 mg and the next higher dose is 30 mg. In one example the lower dose 20 mg and the next higher dose is 40 mg.

In one embodiment, “higher dose” and “next higher dose” are synonyms.

In one embodiment, the “next higher dose” is selected from 5 mg, 10 mg, 20 mg, 30 mg, 40 mg, 60 mg, and 80 mg. In one embodiment, the “next higher dose” is selected from 20 mg, 30 mg, and 40 mg. In one embodiment, the “next higher dose” is 20 mg. In one embodiment, the “next higher dose” is 30 mg. In one embodiment, the “next higher dose” is 40 mg. In one embodiment, the “next higher dose” is 60 mg. In one embodiment, the “next higher dose” is 80 mg.

In one embodiment, the “next lower dose” is selected from 10 mg, 20 mg, and 30 mg. In one embodiment, the “next lower dose” is 10 mg. In one embodiment, the “next lower dose” is 20 mg. In one embodiment, the “next lower dose” is 30 mg.

In some embodiments, the amount of the compound according to formula (I) is adjusted by up-titration. Here, the patient receives a lower dose, which is up-titrated to a higher dose:

In one embodiment, the “lower dose” is 5 mg, and the higher dose is selected from 10 mg, 20 mg, 30 mg, 40 mg, 60 mg and 80 mg. In one embodiment, the “lower dose” is 10 mg, and the higher dose is selected from 20 mg, 30 mg, 40 mg, 60 mg and 80 mg. In one embodiment, the “lower dose” is 20 mg, and the higher dose is selected from 30 mg, 40 mg, 60 mg and 80 mg. In one embodiment, the “lower dose” is 30 mg, and the higher dose is selected from 40 mg, 60 mg and 80 mg. In one embodiment, the “lower dose” is 40 mg, and the higher dose is selected from 60 mg and 80 mg. In one embodiment, the “lower dose” is 60 mg, and the higher dose is 80 mg. In these embodiments, the “higher dose” can be the “next higher dose”.

In one embodiment, the “lower dose” is 5 mg, and the “higher dose” is 10 mg. In one embodiment, the “lower dose” is 5 mg, and the “higher dose” is 20 mg. In one embodiment, the “lower dose” is 5 mg, and the “higher dose” is 30 mg. In one embodiment, the “lower dose” is 5 mg, and the “higher dose” is 40 mg. In one embodiment, the “lower dose” is 5 mg, and the “higher dose” is 60 mg. In one embodiment, the “lower dose” is 5 mg, and the “higher dose” is 80 mg. In one embodiment, the “lower dose” is 10 mg, and the “higher dose” is 20 mg. In one embodiment, the “lower dose” is 10 mg, and the “higher dose” is 30 mg. In one embodiment, the “lower dose” is 10 mg, and the “higher dose” is 40 mg. In one embodiment, the “lower dose” is 10 mg, and the “higher dose” is 60 mg. In one embodiment, the “lower dose” is 10 mg, and the “higher dose” is 80 mg. In one embodiment, the “lower dose” is 10 mg and the “next higher dose” is 20 mg. In one embodiment, the “lower dose” is 10 mg and the “next higher dose” is 40 mg. In one embodiment, the “lower dose” is 20 mg, and the “higher dose” is 40 mg. In one embodiment, the “lower dose” is 20 mg, and the “higher dose” is 30 mg. In one embodiment, the “lower dose” is 20 mg, and the “next higher dose” is 40 mg. In one embodiment, the “lower dose” is 20 mg, and the “higher dose” is 60 mg. In one embodiment, the “lower dose” is 20 mg, and the “higher dose” is 80 mg. In one embodiment, the “lower dose” is 30 mg, and the “next higher dose” is 40 mg. In one embodiment, the “lower dose” is 30 mg, and the “higher dose” is 60 mg. In one embodiment, the “lower dose” is 30 mg, and the “higher dose” is 80 mg. In one embodiment, the “lower dose” is 40 mg, and the “higher dose” is 60 mg. In one embodiment, the “lower dose” is 40 mg, and the “higher dose” is 80 mg. In one embodiment, the “lower dose” is 60 mg, and the “higher dose” is 80 mg. In these embodiments, the “higher dose” can be the “next higher dose”.

In some embodiments, the amount of the compound according to formula (I) is adjusted by down-titration. Here, the patient receives a higher dose, which is down-titrated to a lower dose:

In one embodiment, the “higher dose” is 80 mg, and the “lower dose” is selected from 2.5 mg, 5 mg, 10 mg, 20 mg, 40 mg, and 60 mg. In one embodiment, the “higher dose” is 60 mg, and the “lower dose” is selected from 5 mg, 10 mg, 20 mg, and 40 mg. In one embodiment, the “higher dose” is 40 mg, and the “lower dose” is selected from 5 mg, 10 mg, and 20 mg. In one embodiment, the “higher dose” is 20 mg, and the “lower dose” is selected from 2.5 mg, 5 mg, and 10 mg. In one embodiment, the “higher dose” is 10 mg, and the “lower dose” is selected from 2.5 mg, and 5 mg. In one embodiment, the “higher dose” is 10 mg, and the “lower dose” is 5 mg. In these embodiments, the “lower dose” can be the “next lower dose”.

In one embodiment, the “higher dose” is 80 mg, and the “lower dose” is 60 mg. In one embodiment, the “higher dose” is 80 mg, and the “lower dose” is 40 mg. In one embodiment, the “higher dose” is 80 mg, and the “lower dose” is 30 mg. In one embodiment, the “higher dose” is 80 mg, and the “lower dose” is 20 mg. In one embodiment, the “higher dose” is 80 mg, and the “lower dose” is 10 mg. In one embodiment, the “higher dose” is 80 mg, and the “lower dose” is 5 mg. In one embodiment, the “higher dose” is 60 mg, and the “lower dose” is 40 mg. In one embodiment, the “higher dose” is 60 mg, and the “lower dose” is 30 mg. In one embodiment, the “higher dose” is 60 mg, and the “lower dose” is 20 mg. In one embodiment, the “higher dose” is 60 mg, and the “lower dose” is 10 mg. In one embodiment, the “higher dose” is 60 mg, and the “lower dose” is 5 mg. In one embodiment, the “higher dose” is 40 mg and the “lower dose” is 30 mg. In one embodiment, the “higher dose” is 40 mg and the “lower dose” is 20 mg. In one embodiment, the “higher dose” is 40 mg and the “lower dose” is 10 mg. In one embodiment, the “higher dose” is 40 mg and the “lower dose” is 5 mg. In one embodiment, the “higher dose” is 20 mg and the “lower dose” is 10 mg. In one embodiment, the “higher dose” is 20 mg and the “lower dose” is 5 mg. In one embodiment, the “higher dose” is 20 mg, and the “lower dose” is 2.5 mg. In one embodiment, the “higher dose” is 10 mg and the “lower dose” is 5 mg. In one embodiment, the “higher dose” is 10 mg, and the “lower dose” is 2.5 mg. In these embodiments, the “lower dose” can be the “next lower dose”.

In one embodiment, the “higher dose” is 40 mg and the “next lower dose” is 20 mg. In one embodiment, the “higher dose” is 40 mg and the “next lower dose” is 10 mg. In one embodiment, the “higher dose” is 40 mg and the “next lower dose” is 5 mg.

In one embodiment, the “higher dose” is 20 mg and the “next lower dose” is 10 mg. In one embodiment, the “higher dose” is 20 mg and the “next lower dose” is 5 mg.

In one embodiment, the “higher dose” is 10 mg and the “next lower dose” is 5 mg. In one embodiment, the “higher dose” is 10 mg and the “next lower dose” is 2.5 mg.

In some embodiments that dose can be adjusted stepwise.

In one embodiment, the dose the patient is receiving is up-titrated stepwise. Stepwise can mean, that the titration is performed in one step. For example, in case that the “lower dose” is 10 mg, and the “higher dose” is 20 mg, than the dose is up-titrated for example from 10 mg to 20 mg. Stepwise can mean, that the titration is performed in at least two steps. For example, in case that the “lower does” is 10 mg, and “the “higher dose” is 40 mg, than the dose is up-titrated for example in the first step from 10 mg to 20 mg and in the second step from 20 mg to 40 mg. Stepwise can mean, that the up-titration is performed in three steps. For example, in case that the “lower does” is 10 mg, and “the “higher dose” is 40 mg, than the dose is up-titrated for example in the first step from 10 mg to 20 mg and in the second step from 20 mg to 30 mg, and in the third step from 30 mg to 40 mg.

In some embodiments that dose can be adjusted stepwise. In one embodiment, the dose the patient is receiving is down-titrated stepwise. Stepwise can mean, that the titration is performed in one step. For example, in case that the “lower dose” is 10 mg, and the “higher dose” is 20 mg, than the dose is down-titrated for example from 20 mg to 10 mg. Stepwise can mean, that the titration is performed in at least two steps. For example, in case that the “lower does” is 10 mg, and “the “higher dose” is 40 mg, than the dose is down-titrated for example in the first step from 40 mg to 20 mg and in the second step from 20 mg to 10 mg. Stepwise can mean, that the up-titration is performed in three steps. For example, in case that the “lower does” is 10 mg, and “the “higher dose” is 40 mg, than the dose is down-titrated for example in the first step from 40 mg to 30 mg and in the second step from 30 mg to 20 mg, and in the third step from 20 mg to 10 mg.

In one embodiment, the patient receives a lower dose of the compound according to formula (I), and
the potassium level is equal to or below 4.5 mmol/L,
   then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.5 mmol/L,
   then the dose is up-titrated to a higher dose stepwise. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.5 mmol/L,
   then the dose is up-titrated to the next higher dose. In this embodiment, the lower dose can be selected from 10 mg and 20 mg; and the next higher dose can be selected from 20 mg and 40 mg.

In one embodiment, the patient receives a lower dose of the compound according to formula (I), and
the potassium level is equal to or below 4.8 mmol/L,
   then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.8 mmol/L,
   then the dose is up-titrated to a higher dose stepwise. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.8 mmol/L,
   then the dose is up-titrated to the next higher dose. In this embodiment, the lower dose can be selected from 10 mg and 20 mg; and the next higher dose can be selected from 20 mg and 40 mg.

In one embodiment, the patient receives a lower dose of the compound according to formula (I), and
the potassium level is equal to or below 5 mmol/L,
   then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 5 mmol/L,
   then the dose is up-titrated to a higher dose stepwise. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 5 mmol/L,
   then the dose is up-titrated to the next higher dose. In this embodiment, the lower dose can be selected from 10 mg and 20 mg; and the next higher dose can be selected from 20 mg and 40 mg.

In one embodiment, the patient receives a lower dose of the compound according to formula (I), and
the potassium level is equal to or below 5.5 mmol/L,
   then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 5.5 mmol/L,
   then the dose is up-titrated to a higher dose stepwise. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 5.5 mmol/L,
   then the dose is up-titrated to the next higher dose. In this embodiment, the lower dose can be selected from 10 mg and 20 mg; and the next higher dose can be selected from 20 mg and 40 mg.

In one embodiment, the patient receives a dose of the compound according to formula (I) in an amount of 10 mg, and the potassium level is equal to or below 4.5 mmol/L, then the dose is up-titrated to 20 mg. In one embodiment, the patient receives a dose of the compound according to formula (I) in an amount of 10 mg, and the potassium level is equal to or below 4.8 mmol/L, then the dose is up-titrated to 20 mg. In one embodiment, the patient receives a dose of the compound according to formula (I) in an amount of 10 mg, and the potassium level is equal to or below 5 mmol/L, then the dose is up-titrated to 20 mg. In one embodiment, the patient receives a dose of the compound according to formula (I) in an amount of 10 mg, and the potassium level is equal to or below 5.5 mmol/L, then the dose is up-titrated to 20 mg.

In one embodiment, the patient receives a dose of the compound according to formula (I) in an amount of 10 mg, and the potassium level is equal to or below 4.5 mmol/L, then the dose is up-titrated to 40 mg. In one embodiment, the patient receives a dose of the compound according to formula (I) in an amount of 10 mg, and the potassium level is equal to or below 4.8 mmol/L, then the dose is up-titrated to 40 mg. In one embodiment, the patient receives a dose of the compound according to formula (I) in an amount of 10 mg, and the potassium level is equal to or below 5 mmol/L, then the dose is up-titrated to 40 mg. In one embodiment, the patient receives a dose of the compound according to formula (I) in an amount of 10 mg, and the potassium level is equal to or below 5.5 mmol/L, then the dose is up-titrated to 40 mg.

In one embodiment, the patient receives a dose of the compound according to formula (I) in an amount of 10 mg, and the potassium level is equal to or below 4.5 mmol/L, then the dose is up-titrated to 40 mg as follows:
Step 1 from 10 mg to 20 mg
Step 2 from 20 mg to 40 mg.

In one embodiment, the patient receives a dose of the compound according to formula (I) in an amount of 10 mg, and the potassium level is equal to or below 4.8 mmol/L, then the dose is up-titrated to 40 mg as follows:
Step 1 from 10 mg to 20 mg
Step 2 from 20 mg to 40 mg.

In one embodiment, the patient receives a dose of the compound according to formula (I) in an amount of 10 mg, and the potassium level is below 5 mmol/L, then the dose is up-titrated to 40 mg as follows:
Step 1 from 10 mg to 20 mg
Step 2 from 20 mg to 40 mg.

In one embodiment, the patient receives a dose of the compound according to formula (I) in an amount of 10 mg, and the potassium level is equal to or below 5.5 mmol/L, then the dose is up-titrated to 40 mg as follows:
Step 1 from 10 mg to 20 mg
Step 2 from 20 mg to 40 mg.

In one embodiment, the patient receives a dose of the compound according to formula (I) in an amount of 20 mg, and the potassium level is equal to or below 4.5 mmol/L, then the dose is up-titrated to 40 mg. In one embodiment, the patient receives a dose of the compound according to formula (I) in an amount of 20 mg, and the potassium level is equal to or below 4.8 mmol/L, then the dose is up-titrated to 40 mg. In one embodiment, the patient receives a dose of the compound according to formula (I) in an amount of 20 mg, and the potassium level is below 5 mmol/L, then the dose is up-titrated to 40 mg. In one embodiment, the patient receives a dose of the compound according to formula (I) in an amount of 20 mg, and the potassium level is equal to or below 5.5 mmol/L, then the dose is up-titrated to 40 mg.

In one embodiment, the dose the patient is receiving is up-titrated depending on the eGFR decrease. In one embodiment, the dose the patient is receiving is up-titrated depending on the eGFR decrease, based on the relative eGFR change from baseline or treatment initiation or prior measurement relative eGFR change from baseline or treatment initiation or prior measurement or treatment initiation or prior measurement.

In one embodiment, the dose the patient is receiving is up-titrated, if the eGFR decrease is equal to or below 5, 10, 15, 20, 25, 30, 35, 40, 45, 50, 57 or 60%, based on the relative eGFR change from baseline or treatment initiation or prior measurement relative eGFR change from baseline or treatment initiation or prior measurement or treatment initiation or prior measurement. In one embodiment, the dose the patient is receiving is up-titrated, if the eGFR decrease is equal to or above 5, 10, 15, 20, 25, 30, 35, 40, 45, 50, 57 or 60%, based on the relative eGFR change from baseline or treatment initiation or prior measurement relative eGFR change from baseline or treatment initiation or prior measurement or treatment initiation or prior measurement. In one embodiment, the dose the patient is receiving is up-titrated, if the eGFR decrease is equal to or below 5, 10, 15, 20, 25, 30, 35, or 40%, based on the relative eGFR change from baseline or treatment initiation or prior measurement relative eGFR change from baseline or treatment initiation or prior measurement or treatment initiation or prior measurement. In one embodiment, the dose the patient is receiving is up-titrated, if the eGFR decrease is equal to or above 5, 10, 15, 20, 25, 30, 35, or 40%, based on the relative eGFR change from baseline or treatment initiation or prior measurement relative eGFR change from baseline or treatment initiation or prior measurement or treatment initiation or prior measurement. In one embodiment, the dose the patient is receiving is up-titrated, if the eGFR decrease is equal to or below 5, 10, 15, 20, 25, or 30%, based on the relative eGFR change from baseline or treatment initiation or prior measurement relative eGFR change from baseline or treatment initiation or prior measurement or treatment initiation or prior measurement. In one embodiment, the dose the patient is receiving is up-titrated, if the eGFR decrease is equal to or above 5, 10, 15, 20, 25, or 30 40%, based on the relative eGFR change from baseline or treatment initiation or prior measurement relative eGFR change from baseline or treatment initiation or prior measurement or treatment initiation or prior measurement.

In one embodiment, the dose the patient is receiving is up-titrated, if the eGFR decrease is equal to or below 25%, based on the relative eGFR change from baseline or treatment initiation or prior measurement. In one embodiment, the dose the patient is receiving is up-titrated, if the eGFR decrease is equal to or below 30%, based on the relative eGFR change from baseline or treatment initiation or prior measurement. In one embodiment, the dose the patient is receiving is up-titrated, if the eGFR decrease is equal to or below 40%, based on the relative eGFR change from baseline or treatment initiation or prior measurement. In one embodiment, the dose the patient is receiving is up-titrated, if the eGFR decrease is equal to or below 50%, based on the relative eGFR change from baseline or treatment initiation or prior measurement. In one embodiment, the dose the patient is receiving is up-titrated, if the eGFR decrease is equal to or below 57%, based on the relative eGFR change from baseline or treatment initiation or prior measurement.

In one embodiment, the dose the patient is receiving is up-titrated, if the eGFR decrease is equal to or above 25%, based on the relative eGFR change from baseline or treatment initiation or prior measurement. In one embodiment, the dose the patient is receiving is up-titrated, if the eGFR decrease is equal to or above 30%, based on the relative eGFR change from baseline or treatment initiation or prior measurement. In one embodiment, the dose the patient is receiving is up-titrated, if the eGFR decrease is equal to or above 40%, based on the relative eGFR change from baseline or treatment initiation or prior measurement. In one embodiment, the dose the patient is receiving is up-titrated, if the eGFR decrease is equal to or above 50%, based on the relative eGFR change from baseline or treatment initiation or prior measurement. In one embodiment, the dose the patient is receiving is up-titrated, if the eGFR decrease is equal to or above 57%, based on the relative eGFR change from baseline or treatment initiation or prior measurement.

In one embodiment, the dose the patient is receiving is up-titrated, if the eGFR decrease is equal to or above 25% to equal to or below 57%, based on the relative eGFR change from baseline or treatment initiation or prior measurement. In one embodiment, the dose the patient is receiving is up-titrated, if the eGFR decrease is equal to or above 25% to equal to or below 50%, based on the relative eGFR change from baseline or treatment initiation or prior measurement. In one embodiment, the dose the patient is receiving is up-titrated, if the eGFR decrease is equal to or above 25% to equal to or below 40%, based on the relative eGFR change from baseline or treatment initiation or prior measurement. In one embodiment, the dose the patient is receiving is up-titrated, if the eGFR decrease is equal to or above 25% to equal to or below 30%, based on the relative eGFR change from baseline or treatment initiation or prior measurement. In one embodiment, the dose the patient is receiving is up-titrated, if the eGFR decrease is equal to or above 30% to equal to or below 57%, based on the relative eGFR change from baseline or treatment initiation or prior measurement. In one embodiment, the dose the patient is receiving is up-titrated, if the eGFR decrease is equal to or above 30% to equal to or below 50%, based on the relative eGFR change from baseline or treatment initiation or prior measurement. In one embodiment, the dose the patient is receiving is up-titrated, if the eGFR decrease is equal to or above 30% to equal to or below 40%, based on the relative eGFR change from baseline or treatment initiation or prior measurement. In one embodiment, the dose the patient is receiving is up-titrated, if the eGFR decrease is equal to or above 40% to equal to or below 57%, based on the relative eGFR change from baseline or treatment initiation or prior measurement. In one embodiment, the dose the patient is receiving is up-titrated, if the eGFR decrease is equal to or above 40% to equal to or below 50%, based on the relative eGFR change from baseline or treatment initiation or prior measurement. In one embodiment, the dose the patient is receiving is up-titrated, if the eGFR decrease is equal to or above 50% to equal to or below 57%, based on the relative eGFR change from baseline or treatment initiation or prior measurement.

In one embodiment, the dose can be adjusted based on the potassium level and/or the eGFR decrease.

In one embodiment, the patient receives a lower dose of the compound according to formula (I), and
the potassium level is equal to or below 4.5 mmol/L,
   and if the eGFR decrease is less than 30%, based on the relative eGFR change from baseline or treatment initiation or prior measurement, then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.5 mmol/L,
   if the eGFR decrease is equal to or below 30%, based on the relative eGFR change from baseline or treatment initiation or prior measurement, then the dose is up-titrated to a higher dose stepwise. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.5 mmol/L,
   and if the eGFR decrease is equal below 30%, based on the relative eGFR change from baseline or treatment initiation or prior measurement, then the dose is up-titrated to the next higher dose. In this embodiment, the lower dose can be selected from 10 mg and 20 mg; and the next higher dose can be selected from 20 mg and 40 mg.

In one embodiment, the patient receives a lower dose of the compound according to formula (I), and
the potassium level is equal to or below 4.8 mmol/L,
   and if the eGFR decrease is equal to or below 30%, based on the relative eGFR change from baseline or treatment initiation or prior measurement, then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.8 mmol/L,
   and if the eGFR decrease is equal to or below 30%, based on the relative eGFR change from baseline or treatment initiation or prior measurement, then the dose is up-titrated to a higher dose stepwise. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.8 mmol/L,
   and if the eGFR decrease is equal to or below 30%, based on the relative eGFR change from baseline or treatment initiation or prior measurement, then the dose is up-titrated to the next higher dose. In this embodiment, the lower dose can be selected from 10 mg and 20 mg; and the next higher dose can be selected from 20 mg and 40 mg.

In one embodiment, the patient receives a lower dose of the compound according to formula (I), and
the potassium level is below 5 mmol/L,
   and if the eGFR decrease is equal to or below 30%, based on the relative eGFR change from baseline or treatment initiation or prior measurement, then the dose is up-titrated to a higher dose stepwise. In this embodiment, the lower dose can be selected from 10 mg and 20 mg; and the higher dose can be selected from 20 mg and 40 mg.

In one embodiment, the patient receives a lower dose of the compound according to formula (I),
the potassium level is below 5 mmol/L,
   and if the eGFR decrease is equal to or below 30%, based on the relative eGFR change from baseline or treatment initiation or prior measurement, then the dose is up-titrated to the next higher dose. In this embodiment, the lower dose can be selected from 10 mg and 20 mg; and the next higher dose can be selected from 20 mg and 40 mg.

In one embodiment, the patient receives a lower dose of the compound according to formula (I), and
the potassium level is equal to or below 5.5 mmol/L,
   and if the eGFR decrease is equal to or below 30%, based on the relative eGFR change from baseline or treatment initiation or prior measurement, then the dose is up-titrated to the next higher dose. In this embodiment, the lower dose can be selected from 10 mg and 20 mg; and the next higher dose can be selected from 20 mg and 40 mg.

In one embodiment, the patient receives a dose of 10 mg of the compound according to formula (I), and
the potassium level is equal to or below 4.5 mmol/L,
   and if the eGFR decrease is equal to or below 30%, based on the relative eGFR change from baseline or treatment initiation or prior measurement, then the dose is up-titrated to 20 mg.

In one embodiment, the patient receives a dose of 10 mg of the compound according to formula (I), and
the potassium level is equal to or below 4.8 mmol/L,
   and if the eGFR decrease is equal to or below 30%, based on the relative eGFR change from baseline or treatment initiation or prior measurement, then the dose is up-titrated to 20 mg.

In one embodiment, the patient receives a dose of 10 mg of the compound according to formula (I), and
the potassium level is below 5 mmol/L,
   and if the eGFR decrease is equal to or below 30%, based on the relative eGFR change from baseline or treatment initiation or prior measurement, then the dose is up-titrated to 20 mg.

In one embodiment, the patient receives a dose of 10 mg of the compound according to formula (I), and
the potassium level is equal to or below 5.5 mmol/L,
   and if the eGFR decrease is equal to or below 30%, based on the relative eGFR change from baseline or treatment initiation or prior measurement, then the dose is up-titrated to 20 mg.

In one embodiment, the patient receives a dose of 10 mg of the compound according to formula (I), and
the potassium level is equal to or below 4.5 mmol/L,
   and if the eGFR decrease is equal to or below 30%, based on the relative eGFR change from baseline or treatment initiation or prior measurement, then the dose is up-titrated to 40 mg.

In one embodiment, the patient receives a dose of 10 mg of the compound according to formula (I), and
the potassium level is equal to or below 4.8 mmol/L,
   and if the eGFR decrease is equal to or below 30%, based on the relative eGFR change from baseline or treatment initiation or prior measurement, then the dose is up-titrated to 40 mg.

In one embodiment, the patient receives a dose of 10 mg of the compound according to formula (I), and
the potassium level is below 5 mmol/L,
   and if the eGFR decrease is equal to or below 30%, based on the relative eGFR change from baseline or treatment initiation or prior measurement, then the dose is up-titrated to 40 mg.

In one embodiment, the patient receives a dose of 10 mg of the compound according to formula (I), and
the potassium level is equal to or below 5.5 mmol/L,
   and if the eGFR decrease is equal to or below 30%, based on the relative eGFR change from baseline or treatment initiation or prior measurement, then the dose is up-titrated to 40 mg.

In one embodiment, the patient receives a dose of 20 mg of the compound according to formula (I), and
the potassium level is equal to or below 4.5 mmol/L,
   and if the eGFR decrease is equal to or below 30%, based on the relative eGFR change from baseline or treatment initiation or prior measurement, then the dose is up-titrated to 40 mg.

In one embodiment, the patient receives a dose of 20 mg of the compound according to formula (I), and
the potassium level is equal to or below 4.8 mmol/L,
   and if the eGFR decrease is equal to or below 30%, based on the relative eGFR change from baseline or treatment initiation or prior measurement, then the dose is up-titrated to 40 mg.

In one embodiment, the patient receives a dose of 20 mg of the compound according to formula (I), and
the potassium level is below 5 mmol/L,
   and if the eGFR decrease is equal to or below 30%, based on the relative eGFR change from baseline or treatment initiation or prior measurement, then the dose is up-titrated to 40 mg.

In one embodiment, the patient receives a dose of 20 mg of the compound according to formula (I), and
the potassium level is equal to or below 5.5 mmol/L,
   and if the eGFR decrease is equal to or below 30%, based on the relative eGFR change from baseline or treatment initiation or prior measurement, then the dose is up-titrated to 40 mg.

In one embodiment, the patient receives a dose of 10 mg of the compound according to formula (I), and
the potassium level is equal to or below 4.5 mmol/L,
   and if the eGFR decrease is equal to or below 30%, based on the relative eGFR change from baseline or treatment initiation or prior measurement, then the dose is up-titrated to 40 mg as follows:
Step 1 10 mg to 20 mg
Step 2 20 mg to 40 mg.

In one embodiment, the patient receives a dose of 10 mg of the compound according to formula (I), and
the potassium level is equal to or below 4.8 mmol/L,
   and if the eGFR decrease is equal to or below 30%, based on the relative eGFR change from baseline or treatment initiation or prior measurement, then the dose is up-titrated to 40 mg as follows:
Step 1 10 mg to 20 mg
Step 2 20 mg to 40 mg.

In one embodiment, the patient receives a dose of 10 mg of the compound according to formula (I), and
the potassium level is below 5 mmol/L,

and if the eGFR decrease is equal to or below 30%, based on the relative eGFR change from baseline or treatment initiation or prior measurement, then the dose is up-titrated to 40 mg as follows:
Step 1 10 mg to 20 mg
Step 2 20 mg to 40 mg.

In one embodiment, the patient receives a dose of 10 mg of the compound according to formula (I), and
the potassium level is equal to or below 5.5 mmol/L,
   and if the eGFR decrease is equal to or below 30%, based on the relative eGFR change from baseline or treatment initiation or prior measurement, then the dose is up-titrated to 40 mg as follows:
Step 1 10 mg to 20 mg
Step 2 20 mg to 40 mg.

In one embodiment, the dose can be adjusted based on the eGFR.

In one embodiment, the patient receives a lower dose and the patient has an eGFR of equal to or below 60 mL/min/1.73 m², the dose is up-titrated to a higher dose. In one embodiment, the patient receives a dose of 10 mg and the patient has an equal to or below 60 mL/min/1.73 m², then the dose is up-titrated to a dose of 20 mg. In one embodiment, the patient receives a dose of 10 mg and the patient has an equal to or below 60 mL/min/1.73 m², then the dose is up-titrated to a dose of 40 mg. In one embodiment, the patient receives a dose of 20 mg and the patient has an equal to or below 60 mL/min/1.73 m², then the dose is up-titrated to a dose of 40 mg.

In one embodiment, the patient receives a lower dose and the patient has an eGFR of equal to or above 60 mL/min/1.73 m², the dose is up-titrated to a higher dose. In one embodiment, the patient receives a dose of 10 mg and the patient has an equal to or above 60 mL/min/1.73 m², the dose is up-titrated to a higher dose of 20 mg. In one embodiment, the patient receives a dose of 10 mg and the patient has an equal to or above 60 mL/min/1.73 m², the dose is up-titrated to a higher dose of 40 mg. In one embodiment, the patient receives a dose of 20 mg and the patient has an equal to or above 60 mL/min/1.73 m², the dose is up-titrated to a higher dose of 40 mg.

In one embodiment, the dose is adjusted based on potassium level and/or eGFR.

In one embodiment, the patient receives a lower dose of the compound according to formula (I), and
the potassium level is equal to or below 4.5 mmol/L,
the patient has an eGFR of equal to or below 60 mL/min/1.73 m²
   then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.5 mmol/L,
the patient has an eGFR of equal to or below 60 mL/min/1.73 m²
   then the dose is up-titrated to the next higher dose. In this embodiment, the lower dose can be selected from 10 mg and 20 mg; and the next higher dose can be selected from 20 mg and 40 mg.

In one embodiment, the patient receives a lower dose of the compound according to formula (I), and
the potassium level is equal to or below 4.8 mmol/L,
the patient has an eGFR of equal to or below 60 mL/min/1.73 m²
   then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.8 mmol/L,
the patient has an eGFR of equal to or below 60 mL/min/1.73 m²
   then the dose is up-titrated to the next higher dose. In this embodiment, the lower dose can be selected from 10 mg and 20 mg; and the next higher dose can be selected from 20 mg and 40 mg.

In one embodiment, the patient receives a lower dose of the compound according to formula (I), and
the potassium level is below 5 mmol/L,
the patient has an eGFR of equal to or below 60 mL/min/1.73 m²
   then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is below 5 mmol/L,
the patient has an eGFR of equal to or below 60 mL/min/1.73 m²
   then the dose is up-titrated to the next higher dose. In this embodiment, the lower dose can be selected from 10 mg and 20 mg; and the next higher dose can be selected from 20 mg and 40 mg.

In one embodiment, the patient receives a lower dose of the compound according to formula (I), and
the potassium level is equal to or below 5.5 mmol/L,
the patient has an eGFR of equal to or below 60 mL/min/1.73 m²
   then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 5.5 mmol/L,
the patient has an eGFR of equal to or below 60 mL/min/1.73 m²
   then the dose is up-titrated to the next higher dose. In this embodiment, the lower dose can be selected from 10 mg and 20 mg; and the next higher dose can be selected from 20 mg and 40 mg.

In one embodiment, the patient receives a dose of 10 mg of the compound according to formula (I), and
the potassium level is equal to or below 4.5 mmol/L,
the patient has an eGFR of equal to or below 60 mL/min/1.73 m²
   then the dose is up-titrated to a dose of 20 mg.

In one embodiment, the patient receives a dose of 10 mg of the compound according to formula (I), and
the potassium level is equal to or below 4.8 mmol/L,
the patient has an eGFR of equal to or below 60 mL/min/1.73 m²
   then the dose is up-titrated to a dose of 20 mg.

In one embodiment, the patient receives a dose of 10 mg of the compound according to formula (I), and
the potassium level is below 5 mmol/L,
the patient has an eGFR of equal to or below 60 mL/min/1.73 m²
   then the dose is up-titrated to dose of 20 mg.

In one embodiment, the patient receives a dose of 10 mg of the compound according to formula (I), and
the potassium level is equal to or below 5.5 mmol/L,
the patient has an eGFR of equal to or below 60 mL/min/1.73 m²
   then the dose is up-titrated to dose of 20 mg.

In one embodiment, the patient receives a lower dose of the compound according to formula (I), and
the potassium level is equal to or below 4.5 mmol/L,
the patient has an eGFR of equal to or above 60 mL/min/1.73 m²
   then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.5 mmol/L,
the patient has an eGFR of equal to or above 60 mL/min/1.73 m²
   then the dose is up-titrated to a higher dose stepwise. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.5 mmol/L,
the patient has an eGFR of equal to or above 60 mL/min/1.73 m²
   then the dose is up-titrated to the next higher dose. In this embodiment, the lower dose can be selected from 10 mg and 20 mg; and the next higher dose can be selected from 20 mg and 40 mg.

In one embodiment, the patient receives a lower dose of the compound according to formula (I), and
the potassium level is equal to or below 4.8 mmol/L,
the patient has an eGFR of equal to or above 60 mL/min/1.73 m²
   then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.8 mmol/L,
the patient has an eGFR of equal to or above 60 mL/min/1.73 m²
   then the dose is up-titrated to a higher dose stepwise. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.8 mmol/L,
the patient has an eGFR of equal to or above 60 mL/min/1.73 m²
   then the dose is up-titrated to the next higher dose. In this embodiment, the lower dose can be selected from 10 mg and 20 mg; and the next higher dose can be selected from 20 mg and 40 mg.

In one embodiment, the patient receives a lower dose of the compound according to formula (I), and
the potassium level is below 5 mmol/L,
the patient has an eGFR of equal to or above 60 mL/min/1.73 m²
   then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is below 5 mmol/L,
the patient has an eGFR of equal to or above 60 mL/min/1.73 m²
   then the dose is up-titrated to a higher dose stepwise. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is below 5 mmol/L,
the patient has an eGFR of equal to or above 60 mL/min/1.73 m²
   then the dose is up-titrated to the next higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 5.5 mmol/L,
the patient has an eGFR of equal to or above 60 mL/min/1.73 m²
   then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 5.5 mmol/L,
the patient has an eGFR of equal to or above 60 mL/min/1.73 m²
   then the dose is up-titrated to a higher dose stepwise. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 5.5 mmol/L,
the patient has an eGFR of equal to or above 60 mL/min/1.73 m²
   then the dose is up-titrated to the next higher dose. In this embodiment, the lower dose can be selected from 10 mg and 20 mg; and the higher dose can be selected from 20 mg and 40 mg.

In one embodiment, the patient receives a dose of 10 mg of the compound according to formula (I), and
the potassium level is equal to or below 4.5 mmol/L,
the patient has an eGFR of equal to or above 60 mL/min/1.73 m²
   then the dose is up-titrated to a dose of 20 mg.

In one embodiment, the patient receives a dose of 10 mg of the compound according to formula (I), and
the potassium level is equal to or below 4.8 mmol/L,
the patient has an eGFR of equal to or above 60 mL/min/1.73 m²
   then the dose is up-titrated to a dose of 20 mg.

In one embodiment, the patient receives a dose of 10 mg of the compound according to formula (I), and
the potassium level is below 5 mmol/L,
the patient has an eGFR of equal to or above 60 mL/min/1.73 m²
   then the dose is up-titrated to a dose of 20 mg.

In one embodiment, the patient receives a dose of 10 mg of the compound according to formula (I), and
the potassium level is equal to or below 5.5 mmol/L,
the patient has an eGFR of equal to or above 60 mL/min/1.73 m²

the dose is up-titrated to a dose of 20 mg.

In one embodiment, the patient receives a dose of 10 mg of the compound according to formula (I), and
the potassium level is equal to or below 4.5 mmol/L,
the patient has an eGFR of equal to or above 60 mL/min/1.73 m²
   then the dose is up-titrated to a dose of 40 mg.

In one embodiment, the patient receives a dose of 10 mg of the compound according to formula (I), and
the potassium level is equal to or below 4.5 mmol/L,
the patient has an eGFR of equal to or above 60 mL/min/1.73 m²
   then the dose is up-titrated to a dose of 40 mg as follows:
Step 1 10 mg to 20 mg
Step 2 20 mg to 40 mg.

In one embodiment, the patient receives a dose of 10 mg of the compound according to formula (I), and
the potassium level is equal to or below 4.8 mmol/L,
the patient has an eGFR of equal to or above 60 mL/min/1.73 m²
   then the dose is up-titrated to a dose of 40 mg.

In one embodiment, the patient receives a dose of 10 mg of the compound according to formula (I), and
the potassium level is equal to or below 4.8 mmol/L,
the patient has an eGFR of equal to or above 60 mL/min/1.73 m²
   then the dose is up-titrated to a dose of 40 mg as follows:
Step 1 10 mg to 20 mg
Step 2 20 mg to 40 mg.

In one embodiment, the patient receives a dose of 10 mg of the compound according to formula (I), and
the potassium level is below 5 mmol/L,
the patient has an eGFR of equal to or above 60 mL/min/1.73 m²
   then the dose is up-titrated to a dose of 40 mg.

In one embodiment, the patient receives a dose of 10 mg of the compound according to formula (I), and
the potassium level is below 5 mmol/L,
the patient has an eGFR of equal to or above 60 mL/min/1.73 m²
   then the dose is up-titrated to a dose of 40 mg. as follows:
Step 1 10 mg to 20 mg
Step 2 20 mg to 40 mg.

In one embodiment, the patient receives a dose of 10 mg of the compound according to formula (I), and
the potassium level is equal to or below 5.5 mmol/L,
the patient has an eGFR of equal to or above 60 mL/min/1.73 m²
   then the dose is up-titrated to a dose of 40 mg.

In one embodiment, the patient receives a dose of 10 mg of the compound according to formula (I), and
the potassium level is equal to or below 5.5 mmol/L,
the patient has an eGFR of equal to or above 60 mL/min/1.73 m²
   then the dose is up-titrated to a dose of 40 mg. as follows:
Step 1 10 mg to 20 mg
Step 2 20 mg to 40 mg.

In one embodiment, the patient receives a dose of 20 mg of the compound according to formula (I), and
the potassium level is equal to or below 4.5 mmol/L,
the patient has an eGFR of equal to or above 60 mL/min/1.73 m²
   then the dose is up-titrated to a dose of 40 mg.

In one embodiment, the patient receives a dose of 20 mg of the compound according to formula (I), and
the potassium level is equal to or below 4.8 mmol/L,
the patient has an eGFR of equal to or above 60 mL/min/1.73 m²
   then the dose is up-titrated to a dose of 40 mg.

In one embodiment, the patient receives a dose of 20 mg of the compound according to formula (I), and
the potassium level is below 5 mmol/L,
the patient has an eGFR of equal to or above 60 mL/min/1.73 m²
   then the dose is up-titrated to a dose of 40 mg.

In one embodiment, the patient receives a dose of 20 mg of the compound according to formula (I), and
the potassium level is equal to or below 5.5 mmol/L,
the patient has an eGFR of equal to or above 60 mL/min/1.73 m²
   then the dose is up-titrated to a dose of 40 mg.

In one embodiment, the dose is adjusted based on potassium level, eGFR and/or eGFR decrease.

In one embodiment, the patient receives a lower dose of the compound according to formula (I), and
the potassium level is equal to or below 4.5 mmol/L,
the patient has an eGFR of equal to or above 60 mL/min/1.73 m², and
the eGFR decrease is equal to or below 30%, based on the relative eGFR change from baseline or treatment initiation or prior measurement, then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.5 mmol/L,
the patient has an eGFR of equal to or above 60 mL/min/1.73 m², and
the eGFR decrease is equal to or below 30%, based on the relative eGFR change from baseline or treatment initiation or prior measurement, then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to the next higher dose. In this embodiment, the lower dose can be selected from 10 mg and 20 mg; and the next higher dose can be selected from 20 mg and 40 mg.

In one embodiment, the patient receives a lower dose of the compound according to formula (I), and
the potassium level is equal to or below 4.8 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.8 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a higher dose stepwise. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 4.8 mmol/L,
the patient has an eGFR of equal to or above 60 mL/min/1.73 m², and
the eGFR decrease is equal to or below 30%, based on the relative eGFR change from baseline or treatment initiation or prior measurement,

then the dose is up-titrated to the next higher dose. In this embodiment, the lower dose can be selected from 10 mg and 20 mg; and the next higher dose can be selected from 20 mg and 40 mg.

In one embodiment, the patient receives a lower dose of the compound according to formula (I), and
the potassium level is below 5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is below 5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a higher dose stepwise. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is below 5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to the next higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 5.5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a higher dose. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 5.5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a higher dose stepwise. In this embodiment, the lower dose can be selected from 10 mg and 20 mg; and the higher dose can be selected from 20 mg and 40 mg.

In one embodiment, the patient receives a lower dose of the compound according to formula (I), and
the potassium level is equal to or below 5.5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to the next higher dose. In this embodiment, the lower dose can be selected from 10 mg and 20 mg; and the higher dose can be selected from 20 mg and 40 mg.

In one embodiment, the patient receives a dose of 10 mg of the compound according to formula (I), and
the potassium level is equal to or below 4.5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a dose of 20 mg.

In one embodiment, the patient receives a dose of 10 mg of the compound according to formula (I), and
the potassium level is equal to or below 4.8 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a dose of 20 mg.

In one embodiment, the patient receives a dose of 10 mg of the compound according to formula (I), and
the potassium level is below 5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a dose of 20 mg.

In one embodiment, the patient receives a dose of 10 mg of the compound according to formula (I), and
the potassium level is equal to or below 5.5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a dose of 20 mg.

In one embodiment, the patient receives a dose of 10 mg of the compound according to formula (I), and
the potassium level is equal to or below 4.5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a dose of 40 mg.

In one embodiment, the patient receives a dose of 10 mg of the compound according to formula (I), and
the potassium level is equal to or below 4.5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a dose of 40 mg as follows:
Step 1 10 mg to 20 mg
Step 2 20 mg to 40 mg.

In one embodiment, the patient receives a dose of 10 mg of the compound according to formula (I), and
the potassium level is equal to or below 4.8 mmol/L,
the patient has an eGFR of equal to or above 60 mL/min/1.73 m², and
the eGFR decrease is equal to or below 30%, based on the relative eGFR change from baseline or treatment initiation or prior measurement,

then the dose is up-titrated to a dose of 40 mg.

In one embodiment, the patient receives a dose of 10 mg of the compound according to formula (I), and
the potassium level is equal to or below 4.8 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a dose of 40 mg as follows:
Step 1 10 mg to 20 mg
Step 2 20 mg to 40 mg.

In one embodiment, the patient receives a dose of 10 mg of the compound according to formula (I), and
the potassium level is below 5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a dose of 40 mg.

In one embodiment, the patient receives a dose of 10 mg of the compound according to formula (I), and
the potassium level is below 5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a dose of 40 mg. as follows:
Step 1 10 mg to 20 mg
Step 2 20 mg to 40 mg.

In one embodiment, the patient receives a dose of 10 mg of the compound according to formula (I), and
the potassium level is equal to or below 5.5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a dose of 40 mg.

In one embodiment, the patient receives a dose of 10 mg of the compound according to formula (I), and
the potassium level is equal to or below 5.5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a dose of 40 mg. as follows:
Step 1 10 mg to 20 mg
Step 2 20 mg to 40 mg.

In one embodiment, the patient receives a dose of 20 mg of the compound according to formula (I), and
the potassium level is equal to or below 4.5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a dose of 40 mg.

In one embodiment, the patient receives a dose of 20 mg of the compound according to formula (I), and
the potassium level is equal to or below 4.8 mmol/L,
the patient has an eGFR of equal to or above 60 mL/min/1.73 m², and
the eGFR decrease is equal to or below 30%, based on the relative eGFR change from baseline or treatment initiation or prior measurement,

the dose is up-titrated to a dose of 40 mg.

In one embodiment, the patient receives a dose of 20 mg of the compound according to formula (I), and
the potassium level is below 5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a dose of 40 mg.

In one embodiment, the patient receives a dose of 20 mg of the compound according to formula (I), and
the potassium level is equal to or below 5.5 mmol/L,
the patient has an eGFR of equal to or above 60 mL/min/1.73 m², and
the eGFR decrease is equal to or below 30%, based on the relative eGFR change from baseline or treatment initiation or prior measurement,
   then the dose is up-titrated to a dose of 40 mg.

In one embodiment, the dose the patient is receiving is down-titrated.

In one embodiment, the dose the patient is receiving is down-titrated depending on the potassium level.

In one embodiment, the dose the patient is receiving is down-titrated, if the potassium level is equal to or above 5.3, 5.4, 5.5, 5.6, 6, 6.5, 7, 7.5 or 8 mmol/L. In one embodiment, the dose the patient is receiving is down-titrated, if the potassium level is equal to or above 5.5 mmol/L. In one embodiment, the dose the patient is receiving is down-titrated, if the potassium level is equal to or above 6 mmol/L. In one embodiment, the dose the patient is receiving is down-titrated, if the potassium level is equal to or above 6.5 mmol/L. In one embodiment, the dose the patient is receiving is down-titrated, if the potassium level is equal to or above 7 mmol/L. In one embodiment, the dose the patient is receiving is down-titrated, if the potassium level is equal to or above 7.5 mmol/L. In one embodiment, the dose the patient is receiving is down-titrated, if the potassium level is equal to or above 8 mmol/L.

equal to or

In one embodiment, the dose the patient is receiving is down-titrated, if the potassium level is equal to or above 5.3, 5.4, 5.5, 5.6, 6, 6.5, 7, 7.5 or 8 mmol/L. In one embodiment, the dose the patient is receiving is down-titrated, if the potassium level is equal to or above 5.3 mmol/L. In one embodiment, the dose the patient is receiving is down-titrated, if the potassium level is above 5.3 mmol/L. In one embodiment, the dose the patient is receiving is down-titrated, if the potassium level is equal to or above 5.4 mmol/L. In one embodiment, the dose the patient is receiving is down-titrated, if the potassium level is above 5.4 mmol/L. In one embodiment, the dose the patient is receiving is down-titrated, if the potassium level is equal to or above 5.5 mmol/L. In one embodiment, the dose the patient is receiving is down-titrated, if the potassium level is above 5.5 mmol/L. In one embodiment, the dose the patient is receiving is down-titrated, if the potassium level is equal to or above 5.6 mmol/L. In one embodiment, the dose the patient is receiving is down-titrated, if the potassium level is above 5.6 mmol/L. In one embodiment, the dose the patient is receiving is down-titrated, if the potassium level is equal to or above 6 mmol/L. In one embodiment, the dose the patient is receiving is down-titrated, if the potassium level is equal to or above 6.5 mmol/L. In one embodiment, the dose the patient is receiving is down-titrated, if the potassium level is equal to or above 7 mmol/L. In one embodiment, the dose the patient is receiving is down-titrated, if the potassium level is equal to or above 7.5 mmol/L. In one embodiment, the dose the patient is receiving is down-titrated, if the potassium level is equal to or above 8 mmol/L.

In one embodiment, the dose the patient is receiving is down-titrated, if the potassium level is equal to or above 5.3 to 8 mmol/L. In one embodiment, the dose the patient is receiving is down-titrated, if the potassium level is equal to or above 5.5 to 7.5 mmol/L. In one embodiment, the dose the patient is receiving is down-titrated, if the potassium level is equal to or above 5.5 to 7 mmol/L. In one embodiment, the dose the patient is receiving is down-titrated, if the potassium level is equal to or above 5.5 to 6.5 mmol/L. In one embodiment, the dose the patient is receiving is down-titrated, if the potassium level is equal to or above 5.5 to 6 mmol/L.

In one embodiment, the patient receives a higher dose of the compound according to formula (I), and
the potassium level is equal to or below 7 mmol/L,
then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
I the potassium level is equal to or above 7 mmol/L,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 7 mmol/L,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 7 mmol/L,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 7 mmol/L,
   then the dose is down-titrated to the next lower dose. In this embodiment, the next lower dose is 10 mg; and the higher dose is 40 mg.

In one embodiment, the patient receives a higher dose of the compound according to formula (I), and
the potassium level is equal to or above 7 mmol/L,
   then the dose is down-titrated to the next lower dose. In this embodiment, the next lower dose is 10 mg; and the higher dose is 20 mg.

In one embodiment, the patient receives a higher dose of the compound according to formula (I), and
the potassium level is equal to or above 7 mmol/L,
   then the dose is down-titrated to the next lower dose. In this embodiment, the next lower dose is 20 mg;
and the higher dose is 40 mg.

In one embodiment, the patient receives a higher dose of the compound according to formula (I), and
the potassium level is equal to or below 6.5 mmol/L,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 6.5 mmol/L,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 6.5 mmol/L,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 6.5 mmol/L,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 6.5 mmol/L,
   then the dose is down-titrated to the next lower dose. In this embodiment, the next lower dose is 10 mg; and the higher dose is 40 mg.

In one embodiment, the patient receives a higher dose of the compound according to formula (I), and
the potassium level is equal to or above 6.5 mmol/L,
   then the dose is down-titrated to the next lower dose. In this embodiment, the next lower dose is 10 mg; and the higher dose is 20 mg.

In one embodiment, the patient receives a higher dose of the compound according to formula (I), and
the potassium level is equal to or above 6.5 mmol/L,
   then the dose is down-titrated to the next lower dose. In this embodiment, the next lower dose is 20 mg; and the higher dose is 40 mg.

In one embodiment, the patient receives a higher dose of the compound according to formula (I), and
the potassium level is equal to or below 6 mmol/L,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 6 mmol/L,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 6 mmol/L,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 6 mmol/L,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 6 mmol/L,
   then the dose is down-titrated to the next lower dose. In this embodiment, the next lower dose is 10 mg; and the higher dose is 40 mg.

In one embodiment, the patient receives a higher dose of the compound according to formula (I), and
the potassium level is equal to or above 6 mmol/L,
   then the dose is down-titrated to the next lower dose. In this embodiment, the next lower dose is 10 mg; and the higher dose is 20 mg.

In one embodiment, the patient receives a higher dose of the compound according to formula (I), and
the potassium level is equal to or above 6 mmol/L,
   then the dose is down-titrated to the next lower dose. In this embodiment, the next lower dose is 20 mg; and the higher dose is 40 mg.

In one embodiment, the patient receives a higher dose of the compound according to formula (I), and
the potassium level is equal to or below 5.5 mmol/L,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L, or
the potassium level is above 5.5 mmol/L,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L, or
the potassium level is above 5.5 mmol/L,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L, or
the potassium level is above 5.5 mmol/L,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L, or
the potassium level is above 5.5 mmol/L,
   then the dose is down-titrated to the next lower dose. In this embodiment, the next lower dose is 10 mg; and the higher dose is 40 mg.

In one embodiment, the patient receives a higher dose of the compound according to formula (I), and
the potassium level is equal to or above 5.5 mmol/L, or
the potassium level is above 5.5 mmol/L,
   then the dose is down-titrated to the next lower dose. In this embodiment, the next lower dose is 10 mg; and the higher dose is 20 mg.

In one embodiment, the patient receives a higher dose of the compound according to formula (I), and
the potassium level is equal to or above 5.5 mmol/L, or
the potassium level is above 5.5 mmol/L,
   then the dose is down-titrated to the next lower dose. In this embodiment, the next lower dose is 20 mg; and the higher dose is 40 mg.

In one embodiment, the patient receives a higher dose of the compound according to formula (I), and
the potassium level is equal to or above 5.4 mmol/L, or
the potassium level is above 5.4 mmol/L,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.4 mmol/L, or
the potassium level is above 5.4 mmol/L,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.4 mmol/L, or
the potassium level is above 5.4 mmol/L,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.4 mmol/L, or
the potassium level is above 5.4 mmol/L,
   then the dose is down-titrated to the next lower dose. In this embodiment, the next lower dose is 10 mg; and the higher dose is 40 mg.

In one embodiment, the patient receives a higher dose of the compound according to formula (I),
the potassium level is equal to or above 5.4 mmol/L, or
the potassium level is above 5.4 mmol/L,
   then the dose is down-titrated to the next lower dose. In this embodiment, the next lower dose is 10 mg; and the higher dose is 20 mg.

In one embodiment, the patient receives a higher dose of the compound according to formula (I), and
the potassium level is equal to or above 5.4 mmol/L, or
the potassium level is above 5.4 mmol/L,
   then the dose is down-titrated to the next lower dose. In this embodiment, the next lower dose is 20 mg; and the higher dose is 40 mg.

In one embodiment, the patient receives a higher dose of the compound according to formula (I), and
the potassium level is equal to or above 5.3 mmol/L, or
the potassium level is above 5.3 mmol/L,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L, or
the potassium level is above 5.3 mmol/L,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L, or
the potassium level is above 5.3 mmol/L,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L, or
the potassium level is above 5.3 mmol/L,
   then the dose is down-titrated to the next lower dose. In this embodiment, the next lower dose is 10 mg; and the higher dose is 40 mg.

In one embodiment, the patient receives a higher dose of the compound according to formula (I), and
the potassium level is equal to or above 5.3 mmol/L, or
the potassium level is above 5.3 mmol/L,
   then the dose is down-titrated to the next lower dose. In this embodiment, the next lower dose is 10 mg; and the higher dose is 20 mg.

In one embodiment, the patient receives a higher dose of the compound according to formula (I), and
the potassium level is equal to or above 5.3 mmol/L, or
the potassium level is above 5.3 mmol/L,
   then the dose is down-titrated to the next lower dose. In this embodiment, the next lower dose is 20 mg; and the higher dose is 40 mg.

In one embodiment, the patient receives a higher dose of the compound according to formula (I), and
the potassium level is equal to or above 5.5 mmol/L and below 6.0, or
the potassium level is above 5.5 mmol/L and below 6.0,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.0, or
the potassium level is above 5.5 mmol/L and below 6.0,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.0, or
the potassium level is above 5.5 mmol/L and below 6.0,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.0, or
the potassium level is above 5.5 mmol/L and below 6.0,
   then the dose is down-titrated to the next lower dose. In this embodiment, the next lower dose is 10 mg; and the higher dose is 40 mg.

In one embodiment, the patient receives a higher dose of the compound according to formula (I), and
the potassium level is equal to or above 5.5 mmol/L and below 6.0, or
the potassium level is above 5.5 mmol/L and below 6.0,
   then the dose is down-titrated to the next lower dose. In this embodiment, the next lower dose is 10 mg; and the higher dose is 20 mg.

In one embodiment, the patient receives a higher dose of the compound according to formula (I), and
the potassium level is equal to or above 5.5 mmol/L and below 6.0, or
the potassium level is above 5.5 mmol/L and below 6.0,
   then the dose is down-titrated to the next lower dose. In this embodiment, the next lower dose is 20 mg; and the higher dose is 40 mg.

In one embodiment, the patient receives a dose of the compound according to formula (I) in an amount of 40 mg, and the potassium level is equal to or below 7 mmol/L, then the dose is down-titrated to 20 mg. In one embodiment, the patient receives a dose of the compound according to formula (I) in an amount of 40 mg, and the potassium level is equal to or below 6.5 mmol/L, then the dose is down-titrated to 20 mg. In one embodiment, the patient receives a dose of the compound according to formula (I) in an amount of 40 mg, and the potassium level is equal to or below 6 mmol/L, then the dose is down-titrated to 20 mg. In one embodiment, the patient receives a dose of the compound according to formula (I) in an amount of 40 mg, and the potassium level is equal to or below 5.5 mmol/L, then the dose is down-titrated to 20 mg.

In one embodiment, the patient receives a dose of the compound according to formula (I) in an amount of 40 mg, and the potassium level is equal to or below 7 mmol/L, then the dose is down-titrated to 10 mg. In one embodiment, the patient receives a dose of the compound according to formula (I) in an amount of 40 mg, and the potassium level is equal to or below 6.5 mmol/L, then the dose is down-titrated to 10 mg. In one embodiment, the patient receives a dose of the compound according to formula (I) in an amount of 40 mg, and the potassium level is equal to or below 6 mmol/L, then the dose is down-titrated to 10 mg. In one embodiment, the patient receives a dose of the compound according to formula (I) in an amount of 40 mg, and the potassium level is equal to or below 5.5 mmol/L, then the dose is down-titrated to 10 mg.

In one embodiment, the patient receives a dose of the compound according to formula (I) in an amount of 20 mg, and the potassium level is equal to or below 7 mmol/L, then the dose is down-titrated to 10 mg. In one embodiment, the patient receives a dose of the compound according to formula (I) in an amount of 20 mg, and the potassium level is equal to or below 6.5 mmol/L, then the dose is down-titrated to 10 mg. In one embodiment, the patient receives a dose of the compound according to formula (I) in an amount of 20 mg, and the potassium level is equal to or below 6 mmol/L, then the dose is down-titrated to 10 mg. In one embodiment, the patient receives a dose of the compound according to formula (I) in an amount of 20 mg, and the potassium level is equal to or below 5.5 mmol/L, then the dose is down-titrated to 10 mg.

In one embodiment, the patient receives a dose of the compound according to formula (I) in an amount of 40 mg, and the potassium level is equal to or below 7 mmol/L, then the dose is down-titrated to 10 mg as follows:
Step 1 from 40 mg to 20 mg
Step 2 from 20 mg to 10 mg.

In one embodiment, the patient receives a dose of the compound according to formula (I) in an amount of 40 mg, and the potassium level is equal to or above 5.5 mmol/L and below 7 mmol/L, then the dose is down-titrated to 10 mg as follows:
Step 1 from 40 mg to 20 mg
Step 2 from 20 mg to 10 mg.

In one embodiment, the patient receives a dose of the compound according to formula (I) in an amount of 40 mg, and the potassium level is equal to or below 6.5 mmol/L, then the dose is down-titrated to 10 mg as follows:
Step 1 from 40 mg to 20 mg
Step 2 from 20 mg to 10 mg.

In one embodiment, the patient receives a dose of the compound according to formula (I) in an amount of 40 mg, and the potassium level is equal to or above 5.5 mmol/L and below 6.5 mmol/L, then the dose is down-titrated to 10 mg as follows:
Step 1 from 40 mg to 20 mg
Step 2 from 20 mg to 10 mg.

In one embodiment, the patient receives a dose of the compound according to formula (I) in an amount of 40 mg, and the potassium level is equal to or below 6 mmol/L, then the dose is down-titrated to 10 mg as follows:
Step 1 from 40 mg to 20 mg
Step 2 from 20 mg to 10 mg.

In one embodiment, the patient receives a dose of the compound according to formula (I) in an amount of 40 mg, and the potassium level is equal to or above 5.5 mmol/L and below 6 mmol/L, then the dose is down-titrated to 10 mg as follows:
Step 1 from 40 mg to 20 mg
Step 2 from 20 mg to 10 mg.

In one embodiment, the patient receives a dose of the compound according to formula (I) in an amount of 40 mg, and the potassium level is equal to or below 5.5 mmol/L, then the dose is down-titrated to 10 mg as follows:
Step 1 from 40 mg to 20 mg
Step 2 from 20 mg to 10 mg.

In one embodiment, the patient receives a dose of the compound according to formula (I) in an amount of 40 mg, and the potassium level is equal to or above 5.3 mmol/L and below 5.5 mmol/L, then the dose is down-titrated to 10 mg as follows:
Step 1 from 40 mg to 20 mg
Step 2 from 20 mg to 10 mg.

In one embodiment, the dose the patient is receiving is down-titrated depending on eGFR decrease.

In one embodiment, the dose the patient is receiving is down-titrated depending on eGFR decrease, based on the relative eGFR change from baseline or treatment initiation or prior measurement.

In one embodiment, the dose the patient is receiving is down-titrated, if the eGFR decrease is selected from equal to or above 20, 25, 30, 35, 40, 45, 50, 55, and 57%, based on the relative eGFR change from baseline or treatment initiation or prior measurement. In one embodiment, the dose the patient is receiving is down-titrated, if the eGFR decrease is equal to or above 25%, based on the relative eGFR change from baseline or treatment initiation or prior measurement. In one embodiment, the dose the patient is receiving is down-titrated, if the eGFR decrease is equal to or above 30%, based on the relative eGFR change from baseline or treatment initiation or prior measurement. In one embodiment, the dose the patient is receiving is down-titrated, if the eGFR decrease is equal to or above 40%, based on the relative eGFR change from baseline or treatment initiation or prior measurement. In one embodiment, the dose the patient is receiving is down-titrated, if the eGFR decrease is equal to or above 50%, based on the relative eGFR change from baseline or treatment initiation or prior measurement. In one embodiment, the dose the patient is receiving is down-titrated, if the eGFR decrease is equal to or above 57%, based on the relative eGFR change from baseline or treatment initiation or prior measurement.

In one embodiment, the dose the patient is receiving is down-titrated, if the eGFR decrease is equal to or above 25% to equal to or below 57%, based on the relative eGFR change from baseline or treatment initiation or prior measurement. In one embodiment, the dose the patient is receiving is down-titrated, if the eGFR decrease is equal to or above 25% to equal to or below 55%, based on the relative eGFR change from baseline or treatment initiation or prior measurement. In one embodiment, the dose the patient is receiving is down-titrated, if the eGFR decrease is equal to or above 25% to equal to or below 50%, based on the relative eGFR change from baseline or treatment initiation or prior measurement.

In one embodiment, the dose the patient is receiving is down-titrated, if the eGFR decrease is equal to or above 25% to equal to or below 45%, based on the relative eGFR change from baseline or treatment initiation or prior measurement. In one embodiment, the dose the patient is receiving is down-titrated, if the eGFR decrease is equal to or above 25% to equal to or below 40%, based on the relative eGFR change from baseline or treatment initiation or prior measurement. In one embodiment, the dose the patient is receiving is down-titrated, if the eGFR decrease is equal to or above 25% to equal to or below 35%, based on the relative eGFR change from baseline or treatment initiation or prior measurement. In one embodiment, the dose the patient is receiving is down-titrated, if the eGFR decrease is equal to or above 25% to equal to or below 30%, based on the relative eGFR change from baseline or treatment initiation or prior measurement.

In one embodiment, the dose the patient is receiving is down-titrated, if the eGFR decrease is equal to or above 30% to equal to or below 57%, based on the relative eGFR change from baseline or treatment initiation or prior measurement. In one embodiment, the dose the patient is receiving is down-titrated, if the eGFR decrease is equal to or above 30% to equal to or below 55%, based on the relative eGFR change from baseline or treatment initiation or prior measurement. In one embodiment, the dose the patient is receiving is down-titrated, if the eGFR decrease is equal to or above 30% to equal to or below 50%, based on the relative eGFR change from baseline or treatment initiation or prior measurement. In one embodiment, the dose the patient is receiving is down-titrated, if the eGFR decrease is equal to or above 30% to equal to or below 45%, based on the relative eGFR change from baseline or treatment initiation or prior measurement. In one embodiment, the dose the patient is receiving is down-titrated, if the eGFR decrease is equal to or above 30% to equal to or below 40%, based on the relative eGFR change from baseline or treatment initiation or prior measurement. In one embodiment, the dose the patient is receiving is down-titrated, if the eGFR decrease is equal to or above 30% to equal to or below 35%, based on the relative eGFR change from baseline or treatment initiation or prior measurement. In one embodiment, the dose the patient is receiving is down-titrated, if the eGFR decrease is equal to or above 35% to equal to or below 57%, based on the relative eGFR change from baseline or treatment initiation or prior measurement. In one embodiment, the dose the patient is receiving is down-titrated, if the eGFR decrease is equal to or above 35% to equal to or below 55%, based on the relative eGFR change from baseline or treatment initiation or prior measurement. In one embodiment, the dose the patient is receiving is down-titrated, if the eGFR decrease is equal to or above 35% to equal to or below 50%, based on the relative eGFR change from baseline or treatment initiation or prior measurement. In one embodiment, the dose the patient is receiving is down-titrated, if the eGFR decrease is equal to or above 35% to equal to or below 45%, based on the relative eGFR change from baseline or treatment initiation or prior measurement.

In one embodiment, the dose the patient is receiving is down-titrated, if the eGFR decrease is equal to or above 35% to equal to or below 40%, based on the relative eGFR change from baseline or treatment initiation or prior measurement. In one embodiment, the dose the patient is receiving is down-titrated, if the eGFR decrease is equal to or above 40% to equal to or below 57%, based on the relative eGFR change from baseline or treatment initiation or prior measurement. In one embodiment, the dose the patient is receiving is down-titrated, if the eGFR decrease is equal to or above 40% to equal to or below 55%, based on the relative eGFR change from baseline or treatment initiation or prior measurement. In one embodiment, the dose the patient is receiving is down-titrated, if the eGFR decrease is equal to or above 40% to equal to or below 50%, based on the relative eGFR change from baseline or treatment initiation or prior measurement. In one embodiment, the dose the patient is receiving is down-titrated, if the eGFR decrease is equal to or above 40% to equal to or below 45%, based on the relative eGFR change from baseline or treatment initiation or prior measurement. In one embodiment, the dose the patient is receiving is down-titrated, if the eGFR decrease is equal to or above 45% to equal to or below 57%, based on the relative eGFR change from baseline or treatment initiation or prior measurement. In one embodiment, the dose the patient is receiving is down-titrated, if the eGFR decrease is equal to or above 45% to equal to or below 55%, based on the relative eGFR change from baseline or treatment initiation or prior measurement. In one embodiment, the dose the patient is receiving is down-titrated, if the eGFR decrease is equal to or above 45% to equal to or below 50%, based on the relative eGFR change from baseline or treatment initiation or prior measurement. In one embodiment, the dose the patient is receiving is down-titrated, if the eGFR decrease is equal to or above 50% to equal to or below 57%, based on the relative eGFR change from baseline or treatment initiation or prior measurement. In one embodiment, the dose the patient is receiving is down-titrated, if the eGFR decrease is equal to or above 50% to equal to or below 55%, based on the relative eGFR change from baseline or treatment initiation or prior measurement.

In one embodiment, the dose the patient is receiving is down-titrated, if the eGFR decrease is equal to or above 55% to equal to or below 57%, based on the relative eGFR change from baseline or treatment initiation or prior measurement.

In one embodiment, the dose is adjusted based on the potassium level and/or the eGFR decrease.

In one embodiment, the patient receives a higher dose of the compound according to formula (I), and
the potassium level is equal to or below 7 mmol/L,
the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and

the potassium level is equal to or above 5.5 mmol/L and below 7 mmol/L,
the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a dose of the compound according to formula (I) in an amount of 40 mg, the eGFR decrease is equal to or above 30%, based on the relative eGFR change from baseline or treatment initiation or prior measurement, and the potassium level is equal to or below 7 mmol/L or equal to or above 5.5 mmol/L and below 7 mmol/L, then the dose is down-titrated to 20 mg. In one embodiment, the patient receives a dose of the compound according to formula (I) in an amount of 40 mg, the eGFR decrease is equal to or above 30%, based on the relative eGFR change from baseline or treatment initiation or prior measurement, and the potassium level is equal to or below 6.5 mmol/L or equal to or above 5.5 mmol/L and
below 6.5 mmol/L, then the dose is down-titrated to 20 mg. In one embodiment, the patient receives a dose of the compound according to formula (I) in an amount of 40 mg, the eGFR decrease is equal to or above 30%, based on the relative eGFR change from baseline or treatment initiation or prior measurement, and the potassium level is equal to or below 6 mmol/L or equal to or above 5.5 mmol/L and below 6 mmol/L, then the dose is down-titrated to 20 mg. In one embodiment, the patient receives a dose of the compound according to formula (I) in an amount of 40 mg, the eGFR decrease is equal to or above 30%, based on the relative eGFR change from baseline or treatment initiation or prior measurement, and the potassium level is equal to or below 5.5 mmol/L or equal to or above 5.3 mmol/L and below 5.5 mmol/L, then the dose is down-titrated to 20 mg.

In one embodiment, the patient receives a dose of the compound according to formula (I) in an amount of 40 mg, the eGFR decrease is equal to or above 30%, based on the relative eGFR change from baseline or treatment initiation or prior measurement, and the potassium level is equal to or below 7 mmol/L or equal to or above 5.5 mmol/L and below 7 mmol/L, then the dose is down-titrated to 10 mg. In one embodiment, the patient receives a dose of the compound according to formula (I) in an amount of 40 mg, the eGFR decrease is equal to or above 30%, based on the relative eGFR change from baseline or treatment initiation or prior measurement, and the potassium level is equal to or below 6.5 mmol/L or equal to or above 5.5 mmol/L and
below 6.5 mmol/L, then the dose is down-titrated to 10 mg. In one embodiment, the patient receives a dose of the compound according to formula (I) in an amount of 40 mg, the eGFR decrease is equal to or above 30%, based on the relative eGFR change from baseline or treatment initiation or prior measurement, and the potassium level is equal to or below 6 mmol/L or equal to or above 5.5 mmol/L and below 6 mmol/L, then the dose is down-titrated to 10 mg. In one embodiment, the patient receives a dose of the compound according to formula (I) in an amount of 40 mg, the eGFR decrease is equal to or above 30%, based on the relative eGFR change from baseline or treatment initiation or prior measurement, and the potassium level is equal to or below 5.5 mmol/L or equal to or above 5.3 mmol/L and below 5.5 mmol/L, then the dose is down-titrated to 10 mg.

In one embodiment, the patient receives a dose of the compound according to formula (I) in an amount of 20 mg, the eGFR decrease is equal to or above 30%, based on the relative eGFR change from baseline or treatment initiation or prior measurement, and the potassium level is equal to or below 7 mmol/L or equal to or above 5.5 mmol/L and below 7 mmol/L, then the dose is down-titrated to 10 mg. In one embodiment, the patient receives a dose of the compound according to formula (I) in an amount of 20 mg, the eGFR decrease is equal to or above 30%, based on the relative eGFR change from baseline or treatment initiation or prior measurement, and the potassium level is equal to or below 6.5 mmol/L or equal to or above 5.5 mmol/L and below 6.5 mmol/L, then the dose is down-titrated to 10 mg. In one embodiment, the patient receives a dose of the compound according to formula (I) in an amount of 20 mg, the eGFR decrease is equal to or above 30%, based on the relative eGFR change from baseline or treatment initiation or prior measurement, and the potassium level is equal to or below 6 mmol/L or equal to or above 5.5 mmol/L and below 6 mmol/L, then the dose is down-titrated to 10 mg. In one embodiment, the patient receives a dose of the compound according to formula (I) in an amount of 20 mg, the eGFR decrease is equal to or above 30%, based on the relative eGFR change from baseline or treatment initiation or prior measurement, and the potassium level is equal to or below 5.5 mmol/L or equal to or above 5.3 mmol/L and below 5.5 mmol/L, then the dose is down-titrated to 10 mg.

In one embodiment, the patient receives a dose of the compound according to formula (I) in an amount of 40 mg, the eGFR decrease is equal to or above 30%, based on the relative eGFR change from baseline or treatment initiation or prior measurement, and the potassium level is equal to or below 7 mmol/L or equal to or above equal to or 5.5 mmol/L, then the dose is down-titrated to 10 mg as follows:
Step 1 from 40 mg to 20 mg
Step 2 from 20 mg to 10 mg.

In one embodiment, the patient receives a dose of the compound according to formula (I) in an amount of 40 mg, the eGFR decrease is equal to or above 30%, based on the relative eGFR change from baseline or treatment initiation or prior measurement, and the potassium level is equal to or below 6.5 mmol/L or equal to or above 5.5 mmol/L and below 6.5 mmol/L, then the dose is down-titrated to 10 mg as follows:
Step 1 from 40 mg to 20 mg
Step 2 from 20 mg to 10 mg.

In one embodiment, the patient receives a dose of the compound according to formula (I) in an amount of 40 mg, the eGFR decrease is equal to or above 30%, based on the relative eGFR change from baseline or treatment initiation or prior measurement, and the potassium level is equal to or below 6 mmol/L or equal to or above 5.5 mmol/L and below 6 mmol/L, then the dose is down-titrated to 10 mg as follows:
Step 1 from 40 mg to 20 mg
Step 2 from 20 mg to 10 mg.

In one embodiment, the patient receives a dose of the compound according to formula (I) in an amount of 40 mg, the eGFR decrease is equal to or above 30%, based on the relative eGFR change from baseline or treatment initiation or prior measurement, and the potassium level is equal to or below 5.5 mmol/L or equal to or above 5.3 mmol/L and below 5.5 mmol/L, then the dose is down-titrated to 10 mg as follows:
Step 1 from 40 mg to 20 mg
Step 2 from 20 mg to 10 mg.

In one embodiment, the patient receives a dose of the compound according to formula (I) in an amount of 40 mg, the eGFR decrease is equal to or above 40%, based on the relative eGFR change from baseline or treatment initiation or prior measurement, and the potassium level is equal to or below 7 mmol/L or equal to or above 5.5 mmol/L and below 7 mmol/L, then the dose is down-titrated to 20 mg. In one embodiment, the patient receives a dose of the compound according to formula (I) in an amount of 40 mg, the eGFR decrease is equal to or above 40%, based on the relative eGFR change from baseline or treatment initiation or prior measurement, and the potassium level is equal to or below 6.5 mmol/L or equal to or above 5.5 mmol/L and below 6.5 mmol/L, then the dose is down-titrated to 20 mg. In one embodiment, the patient receives a dose of the compound according to formula (I) in an amount of 40 mg, the eGFR decrease is equal to or above 40%, based on the relative eGFR change from baseline or treatment initiation or prior measurement, and the potassium level is equal to or below 6 mmol/L or equal to or above 5.5 mmol/L and below 6 mmol/L, then the dose is down-titrated to 20 mg. In one embodiment, the patient receives a dose of the compound according to formula (I) in an amount of 40 mg, the eGFR decrease is equal to or above 40%, based on the relative eGFR change from baseline or treatment initiation or prior measurement, and the potassium level is equal to or below 5.5 mmol/L or equal to or above 5.3 mmol/L and below 5.5 mmol/L, then the dose is down-titrated to 20 mg.

In one embodiment, the patient receives a dose of the compound according to formula (I) in an amount of 40 mg, the eGFR decrease is equal to or above 40%, based on the relative eGFR change from baseline or treatment initiation or prior measurement, and the potassium level is equal to or below 7 mmol/L or equal to or above 5.5 mmol/L and below 7 mmol/L, then the dose is down-titrated to 10 mg. In one embodiment, the patient receives a dose of the compound according to formula (I) in an amount of 40 mg, the eGFR decrease is equal to or above 40%, based on the relative eGFR change from baseline or treatment initiation or prior measurement, and the potassium level is equal to or below 6.5 mmol/L or equal to or above 5.5 mmol/L and below 6.5 mmol/L, then the dose is down-titrated to 10 mg. In one embodiment, the patient receives a dose of the compound according to formula (I) in an amount of 40 mg, the eGFR decrease is equal to or above 40%, based on the relative eGFR change from baseline or treatment initiation or prior measurement, and the potassium level is equal to or below 6 mmol/L or equal to or above 5.5 mmol/L and below 6 mmol/L, then the dose is down-titrated to 10 mg. In one embodiment, the patient receives a dose of the compound according to formula (I) in an amount of 40 mg, the eGFR decrease is equal to or above 40%, based on the relative eGFR change from baseline or treatment initiation or prior measurement, and the potassium level is equal to or below 5.5 mmol/L or equal to or above 5.3 mmol/L and below 5.5 mmol/L, then the dose is down-titrated to 10 mg.

In one embodiment, the patient receives a dose of the compound according to formula (I) in an amount of 20 mg, the eGFR decrease is equal to or above 40%, based on the relative eGFR change from baseline or treatment initiation or prior measurement, and the potassium level is equal to or below 7 mmol/L or equal to or above 5.5 mmol/L and below 7 mmol/L, then the dose is down-titrated to 10 mg. In one embodiment, the patient receives a dose of the compound according to formula (I) in an amount of 20 mg, the eGFR decrease is equal to or above 40%, based on the relative eGFR change from baseline or treatment initiation or prior measurement, and the potassium level is equal to or below 6.5 mmol/L or equal to or above 5.5 mmol/L and below 6.5 mmol/L, then the dose is down-titrated to 10 mg. In one embodiment, the patient receives a dose of the compound according to formula (I) in an amount of 20 mg, the eGFR decrease is equal to or above 40%, based on the relative eGFR change from baseline or treatment initiation or prior measurement, and the potassium level is equal to or below 6 mmol/L or equal to or above 5.5 mmol/L and below 6 mmol/L, then the dose is down-titrated to 10 mg. In one embodiment, the patient receives a dose of the compound according to formula (I) in an amount of 20 mg, the eGFR decrease is equal to or above 40%, based on the relative eGFR change from baseline or treatment initiation or prior measurement, and the potassium level is equal to or below 5.5 mmol/L or equal to or above 5.3 mmol/L and below 5.5 mmol/L, then the dose is down-titrated to 10 mg.

In one embodiment, the patient receives a dose of the compound according to formula (I) in an amount of 40 mg, the eGFR decrease is equal to or above 40%, based on the relative eGFR change from baseline or treatment initiation or prior measurement, and the potassium level is equal to or below 7 mmol/L or equal to or above 5.5 mmol/L and below 7 mmol/L, then the dose is down-titrated to 10 mg as follows:
Step 1 from 40 mg to 20 mg
Step 2 from 20 mg to 10 mg.

In one embodiment, the patient receives a dose of the compound according to formula (I) in an amount of 40 mg, the eGFR decrease is equal to or above 40%, based on the relative eGFR change from baseline or treatment initiation or prior measurement, and the potassium level is equal to or below 6.5 mmol/L or equal to or above 5.5 mmol/L and below 6.5 mmol/L, then the dose is down-titrated to 10 mg as follows:
Step 1 from 40 mg to 20 mg
Step 2 from 20 mg to 10 mg.

In one embodiment, the patient receives a dose of the compound according to formula (I) in an amount of 40 mg, the eGFR decrease is equal to or above 40%, based on the relative eGFR change from baseline or treatment initiation or prior measurement, and the potassium level is equal to or below 6 mmol/L or equal to or above 5.5 mmol/L and below 6 mmol/L, then the dose is down-titrated to 10 mg as follows:
Step 1 from 40 mg to 20 mg
Step 2 from 20 mg to 10 mg.

In one embodiment, the patient receives a dose of the compound according to formula (I) in an amount of 40 mg, the eGFR decrease is equal to or above 40%, based on the relative eGFR change from baseline or treatment initiation or prior measurement, and the potassium level is equal to or below 5.5 mmol/L or equal to or above 5.3 mmol/L and below 5.5 mmol/L, then the dose is down-titrated to 10 mg as follows:
Step 1 from 40 mg to 20 mg
Step 2 from 20 mg to 10 mg.

In one embodiment, the dose is adjusted based on the potassium level and/or the eGFR.

In one embodiment, the patient receives a higher dose of the compound according to formula (I), and
the potassium level is equal to or below 7 mmol/L,
the patient has an eGFR of equal to or above 60 mL/min/1.73 m², and
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L
the patient has an eGFR of equal to or above 60 mL/min/1.73 m², and

then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or above 60 mL/min/1.73 m², and
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
   the patient has an eGFR of equal to or above 60 mL/min/1.73 m², and then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or above 60 mL/min/1.73 m², and
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or above 60 mL/min/1.73 m², and
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or above 60 mL/min/1.73 m², and
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or above 60 mL/min/1.73 m², and
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or above 60 mL/min/1.73 m², and
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or above 60 mL/min/1.73 m², and then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or above 60 mL/min/1.73 m², and
   then the dose is down-titrated to the next lower dose. In this embodiment, the next lower dose can be selected from 10 mg and 20 mg; and the higher dose can be selected from 20 mg and 40 mg.
   equal to or

In one embodiment, the patient receives a higher dose of the compound according to formula (I), and
the potassium level is equal to or above 5.5 mmol/L and below 6.5 mmol/L,
the patient has an eGFR of equal to or above 60 mL/min/1.73 m², and
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or above 60 mL/min/1.73 m², and
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or above 60 mL/min/1.73 m², and
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or above 60 mL/min/1.73 m², and
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or above 60 mL/min/1.73 m², and
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or above 60 mL/min/1.73 m², and
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or above 60 mL/min/1.73 m², and
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or above 60 mL/min/1.73 m², and
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or above 60 mL/min/1.73 m², and
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or above 60 mL/min/1.73 m², and
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or above 60 mL/min/1.73 m², and
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or above 60 mL/min/1.73 m², and
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or above 60 mL/min/1.73 m², and
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or below 60 mL/min/1.73 m², and
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I),
the potassium level is equal to or above 5.5 mmol/L and below 7 mmol/L,
the patient has an eGFR of equal to or below 60 mL/min/1.73 m², and
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or below 60 mL/min/1.73 m², and
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or below 60 mL/min/1.73 m², and
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or below 60 mL/min/1.73 m², and
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or below 60 mL/min/1.73 m², and
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
   the patient has an eGFR of equal to or below 60 mL/min/1.73 m², and
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or below 60 mL/min/1.73 m², and
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or below 60 mL/min/1.73 m², and
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or below 60 mL/min/1.73 m², and
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or below 60 mL/min/1.73 m², and
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or below 60 mL/min/1.73 m², and
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or below 60 mL/min/1.73 m², and
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or equal to or above 5.5 mmol/L and below 6 mmol/L,
the patient has an eGFR of equal to or below 60 mL/min/1.73 m², and
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or below 60 mL/min/1.73 m², and
   equal to or then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or below 60 mL/min/1.73 m², and
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or below 60 mL/min/1.73 m², and
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or below 60 mL/min/1.73 m², and
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or below 60 mL/min/1.73 m², and
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or below 60 mL/min/1.73 m², and
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or below 60 mL/min/1.73 m², and
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or below 60 mL/min/1.73 m², and then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or below 60 mL/min/1.73 m², and
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or below 60 mL/min/1.73 m², and
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I),
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or above 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I),
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 5.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 5.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 5.5 mmol/L,
the patient has an eGFR of equal to or below 60 mL/min/1.73 m², and the eGFR decrease is equal to or above 30%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or above 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 7 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 7 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 6 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.5 mmol/L and below 6 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to a lower dose stepwise. In this embodiment, the lower dose can be selected from 10 mg and 20 mg; and the higher dose can be selected from 20 mg and 40 mg.

In one embodiment, the patient receives a higher dose of the compound according to formula (I), and
the potassium level is equal to or below 5.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a higher dose of the compound according to formula (I), and
the potassium level is equal to or above 5.3 mmol/L and below 5.5 mmol/L,
the patient has an eGFR of equal to or below 60 mL/min/1.73 m², and the eGFR decrease is equal to or above 40%, based on the relative eGFR change from baseline or treatment initiation or prior measurement,
   then the dose is down-titrated to the next lower dose. In this embodiment, the next lower dose can be selected from 10 mg and 20 mg; and the higher dose can be selected from 20 mg and 40 mg.

In one embodiment, the patient receives a dose of 40 mg of the compound according to formula (I), and
the potassium level is equal to or above 5.5 mmol/L,
the patient has an eGFR of equal to or above 60 mL/min/1.73 m²

then the dose is down-titrated to a dose of 10 mg.

In one embodiment, the patient receives a dose of 40 mg of the compound according to formula (I), and
the potassium level is equal to or above 5.5 mmol/L,
the patient has an eGFR of equal to or above 60 mL/min/1.73 m²
   then the dose is down-titrated to a dose of 10 mg as follows:
Step 1 40 mg to 20 mg
Step 2 20 mg to 10 mg.

In one embodiment, the patient receives a dose of 40 mg of the compound according to formula (I), and
the potassium level is equal to or above 5.5 mmol/L,
the patient has an eGFR of equal to or below 60 mL/min/1.73 m²
   then the dose is down-titrated to a dose of 10 mg.

In one embodiment, the patient receives a dose of 40 mg of the compound according to formula (I), and
the potassium level is equal to or above 5.5 mmol/L,
the patient has an eGFR of equal to or below 60 mL/min/1.73 m²
   then the dose is down-titrated to a dose of 10 mg as follows:
Step 1 40 mg to 20 mg
Step 2 20 mg to 10 mg.

In one embodiment, the patient receives a dose of 40 mg of the compound according to formula (I), and
the potassium level is equal to or above 5.5 mmol/L,
the patient has an eGFR of equal to or above 60 mL/min/1.73 m²
   then the dose is down-titrated to a dose of 20 mg.

In one embodiment, the patient receives a dose of 40 mg of the compound according to formula (I), and

the potassium level is equal to or above 5.5 mmol/L,
the patient has an eGFR of equal to or below 60 mL/min/1.73 m²
   then the dose is down-titrated to a dose of 20 mg.

In one embodiment, the patient receives a dose of 20 mg of the compound according to formula (I), and
the potassium level is equal to or above 5.5 mmol/L,
the patient has an eGFR of equal to or above 60 mL/min/1.73 m²
   then the dose is down-titrated to a dose of 10 mg.

In one embodiment, the patient receives a dose of 20 mg of the compound according to formula (I), and
the potassium level is equal to or above 5.5 mmol/L,
the patient has an eGFR of equal to or below 60 mL/min/1.73 m²
   then the dose is down-titrated to a dose of 10 mg.

In one embodiment, the patient receives a dose of 40 mg of the compound according to formula (I), and
the potassium level is equal to or above 5.5 mmol/L,
the patient has an eGFR of equal to or above 60 mL/min/1.73 m²,
the eGFR decrease is more than 30%, based on the relative eGFR change from baseline or treatment initiation or prior measurement,
   then the dose is down-titrated to a dose of 10 mg.

In one embodiment, the patient receives a dose of 40 mg of the compound according to formula (I), and
the potassium level is equal to or above 5.5 mmol/L,
the patient has an eGFR of equal to or above 60 mL/min/1.73 m²,
the eGFR decrease is more than 30%, based on the relative eGFR change from baseline or treatment initiation or prior measurement,
   then the dose is down-titrated to a dose of 10 mg as follows:

Step 1 40 mg to 20 mg

Step 2 20 mg to 10 mg.

In one embodiment, the patient receives a dose of 40 mg of the compound according to formula (I), and
the potassium level is equal to or above 5.5 mmol/L,
the patient has an eGFR of equal to or below 60 mL/min/1.73 m²,
the eGFR decrease is more than 30%, based on the relative eGFR change from baseline or treatment initiation or prior measurement,
   then the dose is down-titrated to a dose of 10 mg.

In one embodiment, the patient receives a dose of 40 mg of the compound according to formula (I), and
the potassium level is equal to or above 5.5 mmol/L,
the patient has an eGFR of equal to or below 60 mL/min/1.73 m²,
the eGFR decrease is more than 30%, based on the relative eGFR change from baseline or treatment initiation or prior measurement,
   then the dose is down-titrated to a dose of 10 mg as follows:
Step 1 40 mg to 20 mg
Step 2 20 mg to 10 mg.

In one embodiment, the patient receives a dose of 40 mg of the compound according to formula (I), and
the potassium level is equal to or above 5.5 mmol/L,
the patient has an eGFR of equal to or above 60 mL/min/1.73 m²,
the eGFR decrease is more than 30%, based on the relative eGFR change from baseline or treatment initiation or prior measurement,
   then the dose is down-titrated to a dose of 20 mg.

In one embodiment, the patient receives a dose of 40 mg of the compound according to formula (I), and
the potassium level is equal to or above 5.5 mmol/L,
the patient has an eGFR of equal to or below 60 mL/min/1.73 m²,
the eGFR decrease is more than 30%, based on the relative eGFR change from baseline or treatment initiation or prior measurement,
   then the dose is down-titrated to a dose of 20 mg.

In one embodiment, the patient receives a dose of 20 mg of the compound according to formula (I), and
the potassium level is equal to or above 5.5 mmol/L,
the patient has an eGFR of equal to or above 60 mL/min/1.73 m²,
the eGFR decrease is more than 30%, based on the relative eGFR change from baseline or treatment initiation or prior measurement,
   then the dose is down-titrated to a dose of 10 mg.

In one embodiment, the patient receives a dose of 20 mg of the compound according to formula (I), and
the potassium level is equal to or above 5.5 mmol/L,
the patient has an eGFR of equal to or below 60 mL/min/1.73 m²,
the eGFR decrease is more than 30%, based on the relative eGFR change from baseline or treatment initiation or prior measurement,
   then the dose is down-titrated to a dose of 10 mg.

In one embodiment, the patient receives a dose of 40 mg of the compound according to formula (I), and
the potassium level is equal to or above 5.5 mmol/L,
the patient has an eGFR of equal to or above 60 mL/min/1.73 m²,
the eGFR decrease is more than 40%, based on the relative eGFR change from baseline or treatment initiation or prior measurement,
   then the dose is down-titrated to a dose of 10 mg.

In one embodiment, the patient receives a dose of 40 mg of the compound according to formula (I), and
the potassium level is equal to or above 5.5 mmol/L,
the patient has an eGFR of equal to or above 60 mL/min/1.73 m²,
the eGFR decrease is more than 40%, based on the relative eGFR change from baseline or treatment initiation or prior measurement,
   then the dose is down-titrated to a dose of 10 mg as follows:
Step 1 40 mg to 20 mg
Step 2 20 mg to 10 mg.

In one embodiment, the patient receives a dose of 40 mg of the compound according to formula (I), and
the potassium level is equal to or above 5.5 mmol/L,
the patient has an eGFR of equal to or below 60 mL/min/1.73 m²,
the eGFR decrease is more than 40%, based on the relative eGFR change from baseline or treatment initiation or prior measurement,
   then the dose is down-titrated to a dose of 10 mg.

In one embodiment, the patient receives a dose of 40 mg of the compound according to formula (I), and
the potassium level is equal to or above 5.5 mmol/L,
the patient has an eGFR of equal to or below 60 mL/min/1.73 m²,
the eGFR decrease is more than 40%, based on the relative eGFR change from baseline or treatment initiation or prior measurement,
   then the dose is down-titrated to a dose of 10 mg as follows:
Step 1 40 mg to 20 mg
Step 2 20 mg to 10 mg.

In one embodiment, the patient receives a dose of 40 mg of the compound according to formula (I), and
the potassium level is equal to or above 5.5 mmol/L,
the patient has an eGFR of equal to or above 60 mL/min/1.73 m²,
the eGFR decrease is more than 40%, based on the relative eGFR change from baseline or treatment initiation or prior measurement,
   then the dose is down-titrated to a dose of 20 mg.

In one embodiment, the patient receives a dose of 40 mg of the compound according to formula (I), and
the potassium level is equal to or above 5.5 mmol/L,
the patient has an eGFR of equal to or below 60 mL/min/1.73 m²,
the eGFR decrease is more than 40%, based on the relative eGFR change from baseline or treatment initiation or prior measurement,
   then the dose is down-titrated to a dose of 20 mg.

In one embodiment, the patient receives a dose of 20 mg of the compound according to formula (I), and
the potassium level is equal to or above 5.5 mmol/L,
the patient has an eGFR of equal to or above 60 mL/min/1.73 m²,
the eGFR decrease is more than 40%, based on the relative eGFR change from baseline or treatment initiation or prior measurement,
   then the dose is down-titrated to a dose of 10 mg.

In one embodiment, the patient receives a dose of 20 mg of the compound according to formula (I), and
the potassium level is equal to or above 5.5 mmol/L,
the patient has an eGFR of equal to or below 60 mL/min/1.73 m²,
the eGFR decrease is more than 40%, based on the relative eGFR change from baseline or treatment initiation or prior measurement,
   equal to orequal to orthen the dose is down-titrated to a dose of 10 mg.

In one embodiment, the patient receives a dose of the compound according to formula (I) in an amount of between 0.5 to 80 mg, the eGFR decrease is equal to or above 30%, based on the relative eGFR change from baseline or treatment initiation or prior measurement, then the dose is down-titrated. The dose can be down-titrated directly or stepwise.

In one embodiment, the patient receives a dose of the compound according to formula (I) in an amount of between 0.5 to 80 mg, the eGFR decrease is equal to or above 25% to equal to or below 40%, based on the relative eGFR change from baseline or treatment initiation or prior measurement, then the dose is down-titrated. The dose can be down-titrated directly or stepwise.

In one embodiment, the patient receives a dose of the compound according to formula (I) in an amount of between 0.5 to 80 mg, the eGFR decrease is equal to or above 40%, based on the relative eGFR change from baseline or treatment initiation or prior measurement, then the dose is down-titrated. The dose can be down-titrated directly or stepwise.

In one embodiment, the patient receives a dose of the compound according to formula (I) in an amount of between 0.5 to 80 mg, the eGFR decrease is equal to or above 30%, based on the relative eGFR change from baseline or treatment initiation or prior measurement, then the treatment is interrupted.

In one embodiment, the patient receives a dose of the compound according to formula (I) in an amount of between 0.5 to 80 mg, the eGFR decrease is equal to or above 25% to equal to or below 40%, based on the relative eGFR change from baseline or treatment initiation or prior measurement, then the treatment is interrupted.

In one embodiment, the patient receives a dose of the compound according to formula (I) in an amount of between 0.5 to 80 mg, the eGFR decrease is equal to or above 40%, based on the relative eGFR change from baseline or treatment initiation or prior measurement, then the treatment is interrupted.

In one embodiment, the patient receives a dose of the compound according to formula (I) in an amount of 20 mg, 30 mg or 40 mg, the eGFR decrease is equal to or above 30%, based on the relative eGFR change from baseline or treatment initiation or prior measurement, then the dose is down-titrated. The dose can be down-titrated directly or stepwise to 10 mg, 20 mg or 30 mg.

In one embodiment, the patient receives a dose of the compound according to formula (I) in an amount of 20 mg, 30 mg or 40 mg, the eGFR decrease is equal to or above 25% to equal to or below 40%, based on the relative eGFR change from baseline or treatment initiation or prior measurement, then the dose is down-titrated. The dose can be down-titrated directly or stepwise to 10 mg, 20 mg or 30 mg.

In one embodiment, the patient receives a dose of the compound according to formula (I) in an amount of 20 mg, 30 mg or 40 mg, the eGFR decrease is equal to or above 40%, based on the relative eGFR change from baseline or treatment initiation or prior measurement, then the dose is down-titrated. The dose can be down-titrated directly or stepwise to 10 mg, 20 mg or 30 mg.

In one embodiment, the patient receives a dose of the compound according to formula (I) in an amount of 20 mg, 30 mg or 40 mg, the eGFR decrease is equal to or above 30%, based on the relative eGFR change from baseline or treatment initiation or prior measurement, then the treatment is interrupted.

In one embodiment, the patient receives a dose of the compound according to formula (I) in an amount of 20 mg, 30 mg or 40 mg, the eGFR decrease is equal to or above 25% to equal to or below 40%, based on the relative eGFR change from baseline or treatment initiation or prior measurement, then the treatment is interrupted.

In one embodiment, the patient receives a dose of the compound according to formula (I) in an amount of 20 mg, 30 mg or 40 mg, the eGFR decrease is equal to or above 40%, based on the relative eGFR change from baseline or treatment initiation or prior measurement, then the treatment is interrupted.

In one embodiment, the patient receives a dose of the compound according to formula (I) in an amount of between 0.5 to 80 mg, the eGFR decrease is equal to or above 25% to equal to or below 40%, based on the relative eGFR change from baseline or treatment initiation or prior measurement, and receives at least one co-medication, then the dose is down-titrated. The dose can be down-titrated directly or stepwise. Comedications are described above in detail.

In one embodiment, the patient receives a dose of the compound according to formula (I) in an amount of between 0.5 to 80 mg, the eGFR decrease is equal to or above 40%, based on the relative eGFR change from baseline or treatment initiation or prior measurement, and receives at least one co-medication, then the dose is down-titrated. The dose can be down-titrated directly or stepwise. Comedications are described above in detail.

In one embodiment, the patient receives a dose of the compound according to formula (I) in an amount of between 0.5 to 80 mg, the eGFR decrease is equal to or above 30%, based on the relative eGFR change from baseline or treatment initiation or prior measurement, and receives at least one comedication, then the treatment is interrupted. Comedications are described above in detail. In one embodiment thereof, the comedication is selected from loop diuretics, beta-blocker, ACE inhibitor (ACEi), angiotensin-receptor-blocker (ARB), Angiotensin Receptor-Neprilysin Inhibitor (ARNI), Calcium Channel Blockers, Sodium-glucose Cotransporter-2 Inhibitor (SGLT-2i), glucagon-like peptide-1 (GLP-1) agonists, CYP3A4 inducers, CYP3A4 inhibitors and mixtures thereof.

In one embodiment, an amount of the compound according to formula (I) the patient receives an amount of the compound according to formula (I) in an amount of between 0.5 to 80 mg, the eGFR decrease is equal to or above 25% to equal to or below 40%, based on the relative eGFR change from baseline or treatment initiation or prior measurement, and receives at least one comedication, then the treatment is interrupted. In one embodiment thereof, the comedication is selected from loop diuretics, beta-blocker, ACE inhibitor (ACEi), angiotensin-receptor-blocker (ARB), Angiotensin Receptor-Neprilysin Inhibitor (ARNI), Calcium Channel Blockers, Sodium-glucose Cotransporter-2 Inhibitor (SGLT-2i), glucagon-like peptide-1 (GLP-1) agonists, CYP3A4 inducers, CYP3A4 inhibitors and mixtures thereof.

In one embodiment, an amount of the compound according to formula (I) the patient receives an amount of the compound according to formula (I) in an amount of between 0.5 to 80 mg, the eGFR decrease is equal to or above 40%, based on the relative eGFR change from baseline or treatment initiation or prior measurement, and receives at least one comedication, then the treatment is interrupted. In one embodiment thereof, the comedication is selected from loop diuretics, beta-blocker, ACE inhibitor (ACEi), angiotensin-receptor-blocker (ARB), Angiotensin Receptor-Neprilysin Inhibitor (ARNI), Calcium Channel Blockers, Sodium-glucose Cotransporter-2 Inhibitor (SGLT-2i), glucagon-like peptide-1 (GLP-1) agonists, CYP3A4 inducers, CYP3A4 inhibitors and mixtures thereof.

In the following further aspects of the disclosure are described. The embodiments disclosed above also apply to these embodiments.

In the following further embodiments of dose managements or adjustments are described: In one embodiment, at least one sample of the patient is taken and the dose the patient is receiving as follows:
FIRST SAMPLE:
   potassium level is <5 mmol/L: Continue the current dose level (maintain dose the patient is receiving) or increase to the next higher dose
   potassium level is ≥5 to <5.5 mmol/L: Continue the current dose level (maintain dose the patient is receiving)
   potassium level is ≥5.5 to <6 mmol/L: Down-titrate to the next lower dose
   potassium level is ≥6 mmol/L: Interrupt treatment (withhold the treatment with compound of formula (I)).

The dose the patient is receiving can be selected from 2.5, 5, 10, 20, 30 and 40 mg. In one embodiment the dose the patient is receiving is 10 mg. In one embodiment, the dose the patient is receiving is 20 mg. In one embodiment the dose the patient is receiving is 30 mg. In one embodiment the dose the patient is receiving is 40 mg. The “higher dose” can be selected from 10, 20, 30, and 40 mg. In one embodiment, the “higher dose” is 20 mg. In one embodiment, the “higher dose” is 30 mg. In one embodiment, the “higher dose” is 40 mg. The “lower dose” can be selected from 10, 20, and 30 mg. In one embodiment, the “lower dose” is 10 mg. In one embodiment, the “lower dose” is 20 mg. In one embodiment, the “lower dose” is 30 mg.

In one embodiment, at least two samples of the patient are taken and the dose the patient is receiving as follows:
FIRST SAMPLE:
   potassium level is <5 mmol/L: Increase to the next higher dose or continue on current dose level if target dose has been reached
   potassium level is ≥5 to <5.5 mmol/L: Continue the current dose level (maintain dose the patient is receiving)
   potassium level is ≥5.5 to <6 mmol/L: Down-titrate to the next lower dose
   potassium level is ≥6 mmol/L: Interrupt treatment (withhold the treatment with compound of formula (I)) and take a second sample
SECOND SAMPLE:
   potassium level is <5.5 mmol/L: Continue current dose
   potassium level is ≥5.5 mmol/L: Down-titrate to the next lower dose if possible, or interrupt study intervention and recheck potassium level

The dose the patient is receiving can be selected from 2.5, 5, 10, 20, 30 and 40 mg. In one embodiment the dose the patient is receiving is 10 mg. In one embodiment, the dose the patient is receiving is 20 mg. In one embodiment the dose the patient is receiving is 30 mg. In one embodiment the dose the patient is receiving is 40 mg. The “higher dose” can be selected from 10, 20, 30, and 40 mg. In one embodiment, the “higher dose” is 20 mg. In one embodiment, the “higher dose” is 30 mg. In one embodiment, the “higher dose” is 40 mg. The “lower dose” can be selected from 10, 20, and 30 mg. In one embodiment, the “lower dose” is 20 mg. In one embodiment, the “lower dose” is 30 mg. In one embodiment, the “lower dose” is 10 mg.

In one embodiment, at least two samples of the patient are taken and the dose the patient is receiving as follows:
FIRST SAMPLE:
   potassium level is <5 mmol/L: Increase to the next higher dose
   potassium level is ≥5 to <5.5 mmol/L: Continue the current dose level (maintain dose the patient is receiving)
   potassium level is ≥5.5 to <6 mmol/L: Down-titrate to the next lower dose
potassium level is ≥6 mmol/L: Interrupt treatment (withhold the treatment with compound of formula (I)) and take a second sample
SECOND SAMPLE:
   potassium level is <5.5 mmol/L: Restart
   potassium level is ≥5.5 mmol/L: Continue to withhold intervention, further monitoring of potassium level.

The dose the patient is receiving can be selected from 2.5, 5, 10, 20, 30 and 40 mg. In one embodiment the dose the patient is receiving is 10 mg. In one embodiment, the dose the patient is receiving is 20 mg. In one embodiment the dose the patient is receiving is 30 mg. In one embodiment the dose the patient is receiving is 40 mg. The “higher dose” can be selected from 10, 20, 30, and 40 mg. In one embodiment, the “higher dose” is 20 mg. In one embodiment, the “higher dose” is 30 mg. In one embodiment, the “higher dose” is 40 mg. The “lower dose” can be selected from 10, 20, and 30 mg. In one embodiment, the “lower dose” is 20 mg. In one embodiment, the “lower dose” is 30 mg. In one embodiment, the “lower dose” is 10 mg. The dose with which the treatment is restarted can be selected from 10 mg, 20 mg and 40 mg. In one embodiment, the dose with which the treatment is restarted is 10 mg. In one embodiment, the dose with which the treatment is restarted is 20 mg. In one embodiment, the dose with which the treatment is restarted is 30 mg. In one embodiment, the dose with which the treatment is restarted is 40 mg.

In one embodiment, at least two samples of the patient are taken and the dose the patient is receiving as follows:
FIRST SAMPLE:
   potassium level is <5 mmol/L: Increase to the next higher dose
   potassium level is ≥5 to <5.5 mmol/L: Continue the current dose level (maintain dose the patient is receiving)
   potassium level is ≥5.5 to <6 mmol/L: Down-titrate to the next lower dose
   potassium level is ≥6 mmol/L: Interrupt treatment (withhold the treatment with compound of formula (I)) and take a second sample
SECOND SAMPLE:
   potassium level is <5.5 mmol/L: Restart
   potassium level is ≥5.5 mmol/L: Continue to withhold intervention, further monitoring of potassium level. Restart, if potassium level is <5.0 mmol/L

The dose the patient is receiving can be selected from 2.5, 5, 10, 20, 30 and 40 mg. In one embodiment the dose the patient is receiving is 10 mg. In one embodiment, the dose the patient is receiving is 20 mg. In one embodiment the dose the patient is receiving is 30 mg. In one embodiment the dose the patient is receiving is 40 mg. The “higher dose” can be selected from 10, 20, 30, and 40 mg. In one embodiment, the “higher dose” is 20 mg. In one embodiment, the “higher dose” is 30 mg. In one embodiment, the “higher dose” is 40 mg. The “lower dose” can be selected from 10, 20, and 30 mg. In one embodiment, the “lower dose” is 20 mg. In one embodiment, the “lower dose” is 30 mg. In one embodiment, the “lower dose” is 10 mg. The dose with which the treatment is restarted can be selected from 10 mg, 20 mg and 40 mg. In one embodiment, the dose with which the treatment is restarted is 10 mg. In one embodiment, the dose with which the treatment is restarted is 20 mg. In one embodiment, the dose with which the treatment is restarted is 30 mg. In one embodiment, the dose with which the treatment is restarted is 40 mg.

In the following, further embodiments of dose adjustment based on the eGFR decrease are disclosed:

In case the eGFR (mL/min/1.73 m²) decrease is between ≥25% and <40%, potential reversible cause can be checked, e.g.
Concomitant medications known to affect renal function (e.g. NSAIDs, antibiotics),
Adverse event (e.g. urinary infection, urinary retention, dehydration), and/or
Further potential reversible causes if considered clinically appropriate.

In case the eGFR (mL/min/1.73 m²) decrease is ≥40%, potential reversible cause can be checked, as mentioned above, e.g.
Concomitant medications known to affect renal function (e.g. non-steroidal anti-inflammatory drug (NSAIDs), antibiotics),
Adverse event (e.g. urinary infection, urinary retention, dehydration), and/or
Further potential reversible causes if considered clinically appropriate.

In case the eGFR (mL/min/1.73 m²) decrease is ≥40%, the dose the patient is receiving can be adjusted or the treatment can be interrupted. In case the eGFR (mL/min/1.73 m²) decrease is ≥40%, the dose the patient is receiving can be adjusted or the treatment can be interrupted and the following can be checked, e.g.:
Further monitor eGFR/creatinine
If eGFR/creatinine has reached acceptable levels (to be determined for the individual participant), restart study intervention at the next lower dose level (or dose level 1 if the participant was already on this dose), and/or
Re-test at central laboratory after 4 weeks to confirm eGFR decrease of ≥50% or ≥57%.

In one embodiment, the patient
receives a dose selected from 10, 20, 30, and 40 mg, and
eGFR (mL/min/1.73 m²) decrease is ≥40%,
   then the dose is down-titrated.

In one embodiment, the dose is adjusted as follows:
Step (1):
   the patient receives a dose selected from 10, 20, 30, and 40 mg, and
   eGFR (mL/min/1.73 m²) decrease is ≥40%,
Step (2):
   down-titrate.

Many embodiments of down-titration, doses, restart and interruption are described in detail above and can be applied to this embodiment.

In one embodiment, the dose is adjusted as follows:
Step (1):
   the patient receives a dose selected from 10, 20, 30, and 40 mg, and
   eGFR (mL/min/1.73 m²) decrease is ≥40%,
Step (2):
   down-titrate as follows:
      monitor eGFR/creatinine;
      If eGFR/creatinine has reached acceptable levels (to be determined for the individual), and/or
      Re-test to confirm eGFR decrease of ≥50% or ≥57%.

Many embodiments of down-titration, doses, restart and interruption are described in detail above and can be applied to this embodiment.

In one embodiment, the dose is adjusted as follows:
Step (1):
   the patient receives a dose selected from 10, 20, 30, and 40 mg, and
   eGFR (mL/min/1.73 m²) decrease is ≥40%,
Step (2):
   interrupted as follows:
      monitor eGFR/creatinine;
      If eGFR/creatinine has reached acceptable levels (to be determined for the individual), restart study intervention at the next lower dose (or lowest dose level, if the participant was already on this dose), and/or
      Re-test to confirm eGFR decrease of ≥50% or ≥57%.

Many embodiments of down-titration, doses, restart and interruption are described in detail above and can be applied to this embodiment.

In one embodiment, the dose is adjusted as follows:
Step (1):
   the patient receives a dose selected from 10, 20, 30, and 40 mg, and
   eGFR (mL/min/1.73 m²) decrease is ≥40%,
Step (2):
   down-titrated or interrupted as follows:
      monitor eGFR/creatinine;
      If eGFR/creatinine has reached acceptable levels (to be determined for the individual), restart study intervention at the next lower dose (or lowest dose level, if the participant was already on this dose), and/or
      Re-test to confirm eGFR decrease of ≥50% or ≥57%.

In one embodiment, the dose is adjusted as follows:

For patients with heart failure (LVEF ≥40%), the recommended target dose of the compound of formula (I) depends on renal function (eGFR) at initiation of the treatment with the compound of formula (I):
40 mg once daily if eGFR ≥60 mL/min/1.73 m²
20 mg once daily if eGFR ≥25 to <60 mL/min/1.73 m²

After 4 weeks of treatment with the compound of formula (I) and thereafter, the dose is adjusted as follows:
potassium level is <5 mmol/L:
   If on 10 mg once daily, increase to 20 mg once daily, if eGFR has not decreased >30% compared to prior measurement.
   If on 20 mg once daily, either maintain dose, or when eGFR >60 mL/min/1.73 m² at treatment initiation, increase to 40 mg once daily, if eGFR has not decreased >30% compared to prior measurement.
potassium level is 5.0 to <5.5 mmol/L: Continue the current dose level (maintain dose the patient is receiving)
potassium level is 5.5 to <6.0 mmol/L: Down-titrate to the next lower dose (decrease to the next lower dose). Interrupt treatment if on 10 mg once daily (withhold the treatment with compound of formula (I)). Restart at 10 mg once daily if serum potassium <5.5 mmol/L.
potassium level is ≥6 mmol/L: Interrupt treatment (withhold the treatment with compound of formula (I)). Restart at 10 mg once daily if serum potassium <5.5 mmol/L. If repeated measurements ≥5.5 mmol/L, restart at 10 mg once daily when <5.0 mmol/L.

Many embodiments of down-titration, doses, treatment restart and treatment interruption are described in detail above and can be applied to this embodiment.

The dose can be adapted based on the type of heart failure, the left ventricular ejection fraction (LVEF), the Urine Albumin to Creatinine Ratio (UACR), the sodium level, the urea level, the N-terminal prohormone of brain natriuretic peptide (NT-proBNP) level, KDIGO stage, chloride level, uric acid, hematologic biomarkers, cardiac biomarkers and/or the medicaments the patient receives. Embodiments of these examples are described in detail above and further below as well as in the examples below.

In one embodiment, the amount of the compound according to formula (I) can be adjusted based on the comedication that the patient is taking.

In one embodiment, the dose of the compound according to formula (I) is adjusted based on the comedication with a CYP3A4 inducer. CYP3A4 inducers are disclosed in detail including specific examples above. The dose of the compound according to formula (I) can be based on the amount and/or type of the CYP3A4 inducer. Adjustments, including down-titration, up-titration, continuation of the dose or interruption of treatment are disclosed in detail and in many embodiments above.

In one embodiment, the patient receives:
a CYP3A4 inducer and
the compound according to formula (I),
   wherein the dose of the compound according to formula (I) is adjusted by up-titration. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose of between 0.5 mg to 80 mg,
   wherein the dose of the compound according to formula (I) is adjusted by up-titration to a dose of between 5 to 80 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose of 5 to 80 mg,
   wherein the dose of the compound according to formula (I) is adjusted by up-titration to a dose of between above 5 to 80 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose selected from the group consisting of 5 mg, 10 mg, 20 mg, 30 mg, 40 mg and 60 mg,
   wherein the dose is adjusted by up-titration to a dose selected from the group consisting of 10 mg, 20 mg, 30 mg, 40 mg, 60 mg, and 80 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose selected from the group consisting of 5 mg, 10 mg, 20 mg, and 30 mg, wherein the dose is adjusted by up-titration to a dose selected from the group consisting of 10 mg, 20 mg, 30 mg and 40 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives
a CYP3A4 inducer and
the compound according to formula (I) in a dose of 5 mg,
   wherein the dose is adjusted by up-titration to a dose selected from the group consisting of 10 mg, 20 mg, 30 mg, 40 mg, 60 mg, and 80 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose of 5 mg,
   wherein the dose is adjusted by up-titration to a dose selected from the group consisting of 10 mg, 20 mg, 30 mg and 40 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose of 10 mg,
   wherein the dose is adjusted by up-titration to a dose selected from the group consisting of 20 mg, 30 mg, 40 mg, 60 mg, and 80 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose of 10 mg,
   wherein the dose is adjusted by up-titration to a dose selected from the group consisting of 20 mg, 30 mg and 40 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose of 20 mg,
   wherein the dose is adjusted by up-titration to a dose selected from the group consisting of 30 mg, 40 mg, 60 mg, and 80 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose of 20 mg,
   wherein the dose is adjusted by up-titration to a dose selected from the group consisting of 30 mg and 40 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose of 40 mg,
   wherein the dose is adjusted by up-titration to a dose selected from the group consisting of 60 mg and 80 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose of 60 mg,
   wherein the dose is adjusted by up-titration to a dose of 80 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

În one embodiment, the patient receives:
a CYP3A4 inducer and
the compound according to formula (I),
   wherein the dose of the compound according to formula (I) is adjusted by down-titration. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose of between 5 mg to 80 mg,
   wherein the dose of the compound according to formula (I) is adjusted by down-titration to a dose of between 0.5 to below 80 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose selected from 10 mg, 20 mg, 30 mg, 40 mg, 60 mg and 80 mg,
   wherein the dose is adjusted by down-titration to a dose selected from the group consisting of 5 mg, 10 mg, 20 mg, 30 mg, 40 mg, and 60 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose selected from 10 mg, 20 mg, 30 mg and 40 mg,
   wherein the dose is adjusted by down-titration to a dose selected from the group consisting of 5 mg, 10 mg, 20 mg, and 30 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose of 10 mg,
   wherein the dose is adjusted by down-titration to a dose of 5 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose of 20 mg,
   wherein the dose is adjusted by down-titration to a dose selected from the group consisting of 5 mg and 10 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose of 40 mg,
   wherein the dose is adjusted by down-titration to a dose selected from the group consisting of 5 mg, 10 mg, 20 mg and 30 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose of 60 mg,
   wherein the dose is adjusted by down-titration to a dose selected from the group consisting of 5 mg, 10 mg, 20 mg, 30 mg and 40 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the patient receives:
a CYP3A4 inducer and
the compound according to formula (I) in a dose of 80 mg,
   wherein the dose is adjusted by down-titration to a dose selected from the group consisting of 5 mg, 10 mg, 20 mg, 30 mg, 40 mg, and 60 mg. In one embodiment thereof, the CYP3A4 inducer is a strong CYP3A4 inducer. In one embodiment thereof, the CYP3A4 inducer is a moderate CYP3A4 inducer. In one embodiment thereof, the CYP3A4 inducer is a weak CYP3A4 inducer. In one embodiment thereof, the CYP3A4 inducer is a CYP3A4 inducer of unspecified potency.

In one embodiment, the dose of the compound according to formula (I) is adjusted based on the comedication with a CYP3A4 inhibitor. CYP3A4 inhibitors are disclosed in detail including specific examples above. The dose of the compound according to formula (I) can be based on the dose and/or type of the CYP3A4 inhibitor. Adjustments, including down-titration, up-titration, continuation of the dose or interruption of treatment are disclosed in detail and in many embodiments above.

In one embodiment, the patient receives the compound of formula (I) in a dose selected from the group consisting of 5 mg, 10 mg, 20 mg, 30 mg, 40 mg, 60 mg, and 80 mg without concomitant CYP3A4 inhibitor treatment. An additional concomitant use of CYP3A4 inhibitor is started and the amount of the compound of formula (I) is reduced (down-titration) during concomitant use of CYP3A4 inhibitor. The dose reduction (down-titration) can start on same day or 1, 2, or 3 days ahead of concomitant use of CYP3A4 inhibitor. The dose reduction (down-titration) ends on the same day or 1, 2, 3, 4, 5, 6, 7, 8, 9, or 10 days after concomitant use of CYP3A4 inhibitor.

In one embodiment, the patient receives the compound of formula (I) in a dose selected from the group consisting of 40 mg, 60 mg, and 80 mg without concomitant CYP3A4 inhibitor treatment. An additional concomitant use of CYP3A4 inhibitor is started and the amount of the compound of formula (I) is reduced (down-titration) during concomitant use of CYP3A4 inhibitor to 10 or 20 mg. The dose reduction (down-titration) can start on same day or 1, 2, or 3 days ahead of concomitant use of CYP3A4 inhibitor. The dose reduction (down-titration) is performed in one or in several steps. The dose reduction (down-titration) ends on the same day or 1, 2, 3, 4, 5, 6, 7, 8, 9, or 10 days after concomitant use of CYP3A4 inhibitor.

In one embodiment, the patient receives the compound of formula (I) in a dose of 40 mg without concomitant CYP3A4 inhibitor treatment. An additional concomitant use of CYP3A4 inhibitor is started and the amount of the compound of formula (I) is reduced (down-titration) during concomitant use of CYP3A4 inhibitor to 10 or 20 mg. The dose reduction (down-titration) can start on same day or 1, 2, or 3 days ahead of concomitant use of CYP3A4 inhibitor. The dose reduction (down-titration) is performed in one or in several steps. The dose reduction (down-titration) ends on the same day or 1, 2, 3, 4, 5, 6, 7, 8, 9, or 10 days after concomitant use of CYP3A4 inhibitor.

In one embodiment, the patient receives the compound of formula (I) in a dose of 40 mg without concomitant CYP3A4 inhibitor treatment. An additional concomitant use of CYP3A4 inhibitor is started and the amount of the compound of formula (I) is reduced (down-titration) during concomitant use of CYP3A4 inhibitor to 20 mg. The dose reduction (down-titration) can start on same day or 1, 2, or 3 days ahead of concomitant use of CYP3A4 inhibitor. The dose reduction (down-titration) is performed in one or in several steps. The dose reduction (down-titration) ends on the same day or 1, 2, 3, 4, 5, 6, 7, 8, 9, or 10 days after concomitant use of CYP3A4 inhibitor.

In one embodiment, the patient receives the compound of formula (I) in a dose of 40 mg without concomitant CYP3A4 inhibitor treatment. An additional concomitant use of CYP3A4 inhibitor is started and the amount of the compound of formula (I) is reduced (down-titration) during concomitant use of CYP3A4 inhibitor to 10 mg. The dose reduction (down-titration) can start on same day or 1, 2, or 3 days ahead of concomitant use of CYP3A4 inhibitor. The dose reduction (down-titration) is performed in one or in several steps. The dose reduction (down-titration) ends on the same day or 1, 2, 3, 4, 5, 6, 7, 8, 9, or 10 days after concomitant use of CYP3A4 inhibitor.

In one embodiment, the patient receives:
a CYP3A4 inhibitor and
the compound according to formula (I),
   wherein the dose of the compound according to formula (I) is adjusted by up-titration. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose of between 0.5 mg to 80 mg,
   wherein the dose of the compound according to formula (I) is adjusted by up-titration to a dose of between 5 to 80 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose of 5 to 80 mg,
   wherein the dose of the compound according to formula (I) is adjusted by up-titration to a dose of between above 5 to 80 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose selected from the group consisting of 5 mg, 10 mg, 20 mg, 30 mg, 40 mg and 60 mg,
   wherein the dose is adjusted by up-titration to a dose selected from the group consisting of 10 mg, 20 mg, 30 mg, 40 mg, 60 mg, and 80 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose selected from the group consisting of 5 mg, 10 mg, 20 mg, and 30 mg,
   wherein the dose is adjusted by up-titration to a dose selected from the group consisting of 10 mg, 20 mg, 30 mg and 40 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose of 5 mg,
   wherein the dose is adjusted by up-titration to a dose selected from the group consisting of10 mg, 20 mg, 30 mg, 40 mg, 60 mg, and 80 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose of 5 mg,
   wherein the dose is adjusted by up-titration to a dose selected from the group consisting of 10 mg, 20 mg, 30 mg and 40 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose of 10 mg,
   wherein the dose is adjusted by up-titration to a dose selected from the group consisting of 20 mg, 30 mg, 40 mg, 60 mg, and 80 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose of 10 mg,
   wherein the dose is adjusted by up-titration to a dose selected from the group consisting of 20 mg, 30 mg and 40 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives
a CYP3A4 inhibitor and
the compound according to formula (I) in a dose of 20 mg,
   wherein the dose is adjusted by up-titration to a dose selected from the group consisting of 30 mg, 40 mg, 60 mg, and 80 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose of 20 mg,
   wherein the dose is adjusted by up-titration to a dose selected from the group consisting of 30 mg and 40 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose of 40 mg,
   wherein the dose is adjusted by up-titration to a dose selected from the group consisting of 60 mg and 80 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose of 60 mg,
   wherein the dose is adjusted by up-titration to a dose of 80 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I),
   wherein the dose of the compound according to formula (I) is adjusted by down-titration. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose of between 5 mg to 80 mg,
   wherein the amount of the compound according to formula (I) is adjusted by down-titration to a dose of between 0.5 to 80 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose selected from the group consisting of 10 mg, 20 mg, 30 mg, 40 mg, 60 mg and 80 mg,
   wherein the dose is adjusted by down-titration to a dose selected from the group consisting of 2.5, 5 mg, 10 mg, 20 mg, 30 mg, 40 mg, and 60 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose selected from the group consisting of 10 mg, 20 mg, 30 mg and 40 mg,
   wherein the dose is adjusted by down-titration to a dose selected from the group consisting of 2.5 mg, 5 mg, 10 mg, 20 mg, and 30 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose of 10 mg,
   wherein the dose is adjusted by down-titration to a dose selected from the group consisting of 2.5 mg and 5 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose of 20 mg,
   wherein the dose is adjusted by down-titration to a dose selected from the group consisting of 2.5 mg, 5 mg and 10 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose of 40 mg,
   wherein the dose is adjusted by down-titration to a dose selected from the group consisting of 5 mg, 10 mg, 20 mg and 30 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose 40 mg,
   wherein the dose is adjusted by down-titration to a dose selected from the group consisting of 20 mg and 10 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a strong CYP3A4 inhibitor and
the compound according to formula (I) in a dose of 40 mg,
   wherein the dose is adjusted by down-titration to a dose selected from the group consisting of 5 mg, 10 mg, 20 mg and 30 mg.

In one embodiment, the patient receives:
a strong CYP3A4 inhibitor and
the compound according to formula (I) in a dose of 40 mg,
   wherein the dose is adjusted by down-titration to a dose of 20 mg.

In one embodiment, the patient receives:
a strong CYP3A4 inhibitor and
the compound according to formula (I) in a dose of 40 mg,
   wherein the dose is adjusted by down-titration to a dose of 10 mg.

In one embodiment, the patient receives:
a weak CYP3A4 inhibitor and
the compound according to formula (I) in a dose of 40 mg,
   wherein the dose is adjusted by down-titration to a dose of 20 mg.

In one embodiment, the patient receives:
a moderate CYP3A4 inhibitor and
the compound according to formula (I) in a dose of 40 mg,
   wherein the dose is adjusted by down-titration to a dose of 20 mg.

In one embodiment, the patient receives:
a strong CYP3A4 inhibitor and
the compound according to formula (I) in a dose of 20 mg,
   wherein the dose is adjusted by down-titration to a dose selected from the group consisting of 2.5 mg, 5 mg, and 10 mg.

In one embodiment, the patient receives:
a strong CYP3A4 inhibitor and
the compound according to formula (I) in a dose of 20 mg,
   wherein the dose is adjusted by down-titration to a dose selected from the group consisting of 5 mg and 10 mg.

In one embodiment, the patient receives:
a strong CYP3A4 inhibitor and
the compound according to formula (I) in a dose of 20 mg,
   wherein the dose is adjusted by down-titration to a dose of 5 mg.

In one embodiment, the patient receives:
a strong CYP3A4 inhibitor and
the compound according to formula (I) in a dose of 20 mg,
   wherein the dose is adjusted by down-titration to a dose of 10 mg.

In one embodiment, the patient receives:
a strong CYP3A4 inhibitor and
the compound according to formula (I) in a dose of 10 mg,
   wherein the dose is adjusted by down-titration to a dose selected from the group consisting of 2.5 mg and 5 mg.

In one embodiment, the patient receives:
a strong CYP3A4 inhibitor and
the compound according to formula (I) in a dose of 10 mg,
   wherein the dose is adjusted by down-titration to a dose of 2.5 mg.

In one embodiment, the patient receives:
a strong CYP3A4 inhibitor and
the compound according to formula (I) in a dose of 10 mg,
   wherein the dose is adjusted by down-titration to a dose of 5 mg.

In one embodiment, the patient receives:
a moderate CYP3A4 inhibitor and
the compound according to formula (I) in a dose of 40 mg,
   wherein the dose is adjusted by down-titration to a dose selected from the group consisting of 10 mg, 20 mg and 30 mg.

In one embodiment, the patient receives:
a moderate CYP3A4 inhibitor and
the compound according to formula (I) in a dose of 40 mg,
   wherein the dose is adjusted by down-titration to a dose selected from the group consisting of 10 mg and 20 mg.

In one embodiment, the patient receives:
a moderate CYP3A4 inhibitor and
the compound according to formula (I) in a dose of 40 mg,
   wherein the dose is adjusted by down-titration to a dose of 10 mg,
In one embodiment, the patient receives:
a moderate CYP3A4 inhibitor and
the compound according to formula (I) in a dose of 40 mg,
   wherein the dose is adjusted by down-titration to a dose of 20 mg.

In one embodiment, the patient receives:
a moderate CYP3A4 inhibitor and
the compound according to formula (I) in a dose of 10 mg,
   wherein the dose is adjusted by down-titration to a dose selected from the group consisting of 2.5 mg and 5 mg.

In one embodiment, the patient receives:
a moderate CYP3A4 inhibitor and
the compound according to formula (I) in a dose of 10 mg,
   wherein the dose is adjusted by down-titration to a dose of 5 mg.

In one embodiment, the patient receives:
a weak CYP3A4 inhibitor and
the compound according to formula (I) in a dose of 40 mg,
   wherein the dose is adjusted by down-titration to a dose selected from the group consisting of 20 mg and 30 mg.

In one embodiment, the patient receives:
a weak CYP3A4 inhibitor and
the compound according to formula (I) in a dose of 40 mg,
   wherein the dose is adjusted by down-titration to a dose of 20 mg.

In one embodiment, the patient receives:
a weak CYP3A4 inhibitor and
the compound according to formula (I) in a dose of 20 mg,
   wherein the dose is adjusted by down-titration to a dose selected from the group consisting of 5 mg and 10 mg.

In one embodiment, the patient receives:
a weak CYP3A4 inhibitor and
the compound according to formula (I) in a dose of 20 mg,
   wherein the dose is adjusted by down-titration to a dose of 10 mg.

In one embodiment, the patient receives:
a weak CYP3A4 inhibitor and
the compound according to formula (I) in a dose of 10 mg,
   wherein the dose is adjusted by down-titration to a dose selected from the group consisting of 2.5 mg and 5 mg.

In one embodiment, the patient receives:
a weak CYP3A4 inhibitor and
the compound according to formula (I) in a dose of 10 mg,
   wherein the dose is adjusted by down-titration to a dose of 5 mg.

In one embodiment, the patient receives:
a CYP3A4 inhibitor and
the compound according to formula (I) in a dose of 60 mg,
   wherein the dose is adjusted by down-titration to a dose selected from the group consisting of 5 mg, 10 mg, 20 mg, 30 mg and 40 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

In one embodiment, the patient receives:
a CYP3A4 inhibitor and
the compound according to formula (I) in a dose of 80 mg,
   wherein the dose is adjusted by down-titration to a dose selected from the group consisting of 5 mg, 10 mg, 20 mg, 30 mg, 40 mg, and 60 mg. In one embodiment thereof, the CYP3A4 inhibitor is a strong CYP3A4 inhibitor. In one embodiment thereof, the CYP3A4 inhibitor is a moderate CYP3A4 inhibitor. In one embodiment thereof, the CYP3A4 inhibitor is a weak CYP3A4 inhibitor. In one embodiment thereof, the CYP3A4 inhibitor is a CYP3A4 inhibitor of unspecified potency.

The dose the patient receives can be adjusted based on patient data. The patient data can comprise
data indicating the disease the patient is suffering from and/or what stage of the disease the patient is in and/or what symptoms the patient is suffering from and/or how severe one or more symptoms are, and/or
information about the medical history of the patient, including information about pre-existing diseases, hospitalizations, and/or genetic background, and/or
information about patient's medications, including medical intervention parameters such as regular medication, occasional medication, and/or other previous or current medical interventions and/or any adverse reactions to medications, and/or
demographic data, including age, sex, ethnicity, and/or other/further personal information about the patient,
results from physical examinations on the patient, and/or laboratory tests, including body size, body weight, body mass index, fat percentage of the body, muscle percentage of the body, water content of the body, resting heart rate, heart rate variability, sugar concentration in urine, body temperature, thoracic impedance, systolic and/or diastolic arterial peripheral blood pressure, glomerular filtration rate, estimated glomerular filtration rate, urine albumin-to-creatinine ratio, creatinine clearance, blood sugar, blood oxygen saturation, erythrocyte count, hemoglobin content, leukocyte count, platelet count, inflammation values, blood lipids including low-density lipoprotein cholesterol and/or high-density lipoprotein cholesterol (HDL), ions including Na⁺, K⁺ and/or corrected calcium, and/or
information about the patient's condition obtained from the patient.

Embodiments of these examples are described in detail above.

A further aspect of the disclosure is a method of reducing the risk of cardiovascular death in a patient, comprising administering to the patient a therapeutically effective amount of the compound (I)

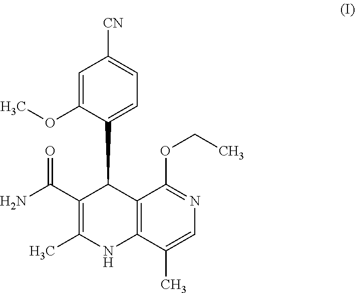 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

A further aspect of the disclosure is a method of improving symptoms of heart failure in a patient, comprising administering to the patient a therapeutically effective amount of the compound (I)

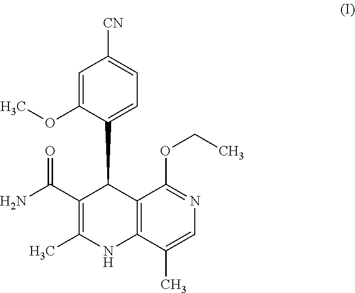 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

A further aspect of the disclosure is a method of preventing worsening of symptoms of heart failure in a patient, comprising administering to the patient a therapeutically effective amount of the compound (I)

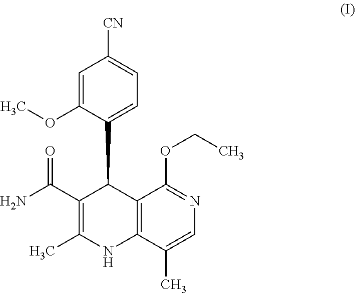 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

A further aspect of the disclosure is a method of reducing the risk of hospitalization for heart failure in a patient, comprising administering to the patient a therapeutically effective amount of the compound (I)

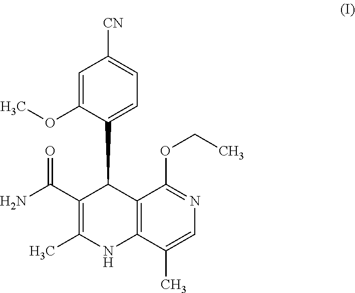 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

A further aspect of the disclosure is a method for treating or preventing symptomatic heart failure with left ventricular ejection fraction (LVEF) of equal to or above 40%, in a patient, comprising administering to the patient a therapeutically effective amount of the compound (I)

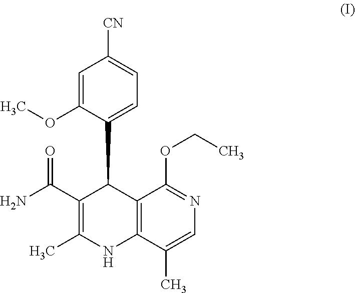 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

A further aspect of the disclosure is a method of reducing the risk of cardiovascular death in patients with heart failure with left ventricular ejection fraction (LVEF) of equal to or above 40%, in a patient, comprising administering to the patient a therapeutically effective amount of the compound (I)

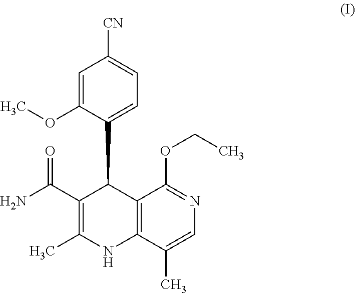 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

A further aspect of the disclosure is a method of reducing the risk of hospitalization for heart failure in patients with heart failure with left ventricular ejection fraction (LVEF) of equal to or above 40%, in a patient, comprising administering to the patient a therapeutically effective amount of the compound (I)

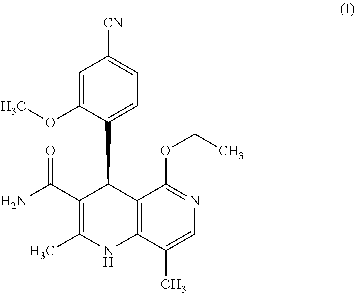 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.

A further aspect of the disclosure is a method of reducing the risk urgent heart failure visits in patients with heart failure with left ventricular ejection fraction (LVEF) of ≥40%, in a patient, comprising administering to the patient a therapeutically effective amount of the compound (I)

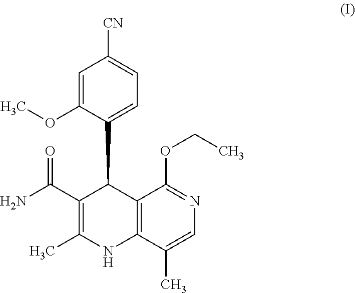 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof. The disclosure also refers to a pharmaceutical composition comprising the compound according to formula (I)

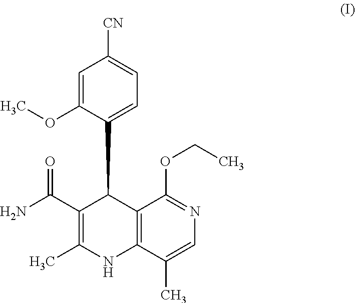 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof that can be used in the methods of treatment as described in detail above. The disclosure also encompasses a pharmaceutical composition comprising a therapeutically effective amount of the compound (I)

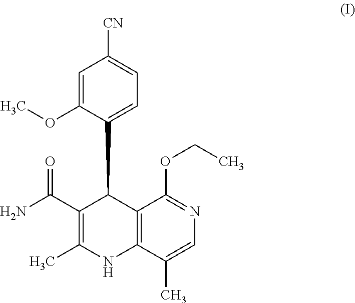 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof that can be used in the methods of treatment as described in detail above.

Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure. Pharmaceutical compositions are known to the person skilled in the art. These can be liquid or solid formulations for example without limitation tablets, coated tablets, capsules, pills, powders, granules, elixirs, tinctures, solution, suspensions, syrups, solid and liquid aerosols and emulsions. Typical pharmaceutical formulations include, but are not limited to, tablets and capsules. These can be used in an intermediate release form or a sustained release form (retard form). Such pharmaceutical formulations are known by the skilled person in the art. In one embodiment, the pharmaceutical composition is a capsule. In one embodiment thereof, the capsule is an intermediate-release capsule. In one embodiment thereof, the capsule is a sustained-release formulation (retard formulation). In one embodiment, the pharmaceutical composition is a tablet. In one embodiment thereof, the tablet is an intermediate-release tablet. In one embodiment thereof, the tablet is a sustained-release formulation (retard formulation).

In another embodiment, the compound according to formula (I) can be tableted with conventional tablet components such as lactose, sucrose and corn starch in combination with binders such as acacia, corn starch and gelatine; disintegrating agents intended to assist the break-up and dissolution of the tablet following administration to the patient such as potato starch, alginic acid, corn starch, and guar gum, gum tragacanth, and acacia; lubricants intended to improve the flow of tablet granulation and to prevent the adhesion of tablet material to the surfaces of the tablet dies and punches, for example talc, stearic acid, or magnesium, calcium or zinc stearate; dyes, coloring agents, and flavoring agents such as peppermint, oil of wintergreen, or cherry flavoring, intended to enhance the aesthetic qualities of the tablets and make them more palatable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials can be present as coatings or to otherwise modify the physical form to achieve a desired release profile.

The disclosure further relates to medicaments which comprise at least one compound according to formula (I), typically together with one or more inert, non-toxic, pharmaceutically suitable excipients, and to the use thereof for the aforementioned purposes.

Methods of Co-Administering Strong and/or Moderate and/or Weak CYP3A4 Inhibitors in Combination with Finerenone

Provided herein are methods of safely and effectively coadministering finerenone with a CYP3A4 inhibitor to a patient in need thereof (e.g., a weak, moderate, or strong CYP3A4 inhibitor), the method comprising administering a reduced dose (reduced relative to the dose recommended for an otherwise similar patient who is not co-administered the CYP3A4 inhibitor) of finerenone.

Provided herein are methods of safely and effectively coadministering finerenone with a strong CYP3A4 inhibitor to a patient in need thereof, the method comprising administering a reduced dose (reduced relative to the dose recommended for an otherwise similar patient who is not co-administered the strong CYP3A4 inhibitor) of finerenone. In embodiments, the reduced dose of finerenone administered according to the disclosed methods is about 10-60% (e.g., about 10%, about 15%, about 20%, about 25%, about 30%, about 35%, about 40%, about 50%, or about 60%) of any dose of finerenone described on the Prescribing Information for finerenone (KERENDIA) (referred to as the “FDA label,” “FDA-approved label” or the “KERENDIA label”), dated July 2025, which is incorporated by reference herein in its entirety for all purposes.

In embodiments, provided herein are methods of coadministering finerenone with a strong CYP3A4 inhibitor to a patient in need thereof, comprising administering about 10-60% (e.g., about 10%, about 15%, about 20%, about 25%, about 30%, about 35%, about 40%, about 50%, or about 60%) of a target daily dose and/or starting dose of finerenone recommended for an otherwise similar patient who is not co-administered the strong CYP3A4 inhibitor. In embodiments, provided herein are methods of coadministering finerenone with a strong CYP3A4 inhibitor to a patient in need thereof, comprising administering about 10-40%. Surprisingly and unexpectedly, Applicant has determined that administering 10-60% of a recommended target daily dose and/or starting daily dose of finerenone in combination with a strong CYP3A4 inhibitor results in comparable exposure to that of a patient that is not administered a strong CYP3A4 inhibitor. (See Examples 28-29a of this Application). Thus, while the FDA-approved label from July 2025 contraindicates coadministration of finerenone with strong CYP3A4 inhibitors, Applicant surprisingly discovered finerenone can be safely and effectively coadministered with strong CYP3A4 inhibitors by reducing the recommended dose of finerenone.

Provided herein are methods of safely and effectively coadministering finerenone with a moderate CYP3A4 inhibitor to a patient in need thereof, the method comprising administering a reduced dose (reduced relative to the dose recommended for an otherwise similar patient who is not co-administered the moderate CYP3A4 inhibitor) of finerenone. In embodiments, the reduced dose of finerenone administered according to the disclosed methods is about 10-60% (e.g., about 10%, about 15%, about 20%, about 25%, about 30%, about 35%, about 40%, about 50%, or about 60%) of any dose of finerenone described on the FDA label for KERENDIA.

Provided herein are methods of safely and effectively coadministering finerenone with a moderate CYP3A4 inhibitor to a patient in need thereof, the method comprising administering a reduced dose (reduced relative to the dose recommended for an otherwise similar patient who is not co-administered the moderate CYP3A4 inhibitor) of finerenone. In embodiments, the reduced dose of finerenone administered according to the disclosed methods is about 10-60% (e.g., about 10%, about 15%, about 20%, about 25%, about 30%, about 35%, about 40%, about 50%, or about 60%) of any dose of finerenone described on the FDA label for KERENDIA.

In embodiments, provided herein are methods of coadministering finerenone with a moderate CYP3A4 inhibitor to a patient in need thereof, comprising administering about 25-75% (e.g., about 25%, about 30%, about 35%, about 40%, about 45%, about 50%, about 55%, about 60%, about 65%, about 70%, or about 75%) of a target daily dose and/or starting dose of finerenone recommended for an otherwise similar patient who is not co-administered the moderate CYP3A4 inhibitor. In embodiments, provided herein are methods of coadministering finerenone with a moderate CYP3A4 inhibitor to a patient in need thereof, comprising administering about 50% of a target daily dose and/or starting dose of finerenone recommended for an otherwise similar patient who is not co-administered the moderate CYP3A4 inhibitor.

In embodiments, the methods provided herein comprise administering from about 2.5 mg to about 30 mg, from about 10 mg to about 30 mg, from about 5 mg to about 15 mg, from about 2.5 mg to about 7.5 mg, from about 2.5 mg to about 25 mg, from about 2.5 mg to about 20 mg, from about 2.5 mg to about 15 mg, from about 2.5 mg to about 10 mg, from about 5 mg to about 30 mg, from about 5 mg to about 25 mg, from about 5 mg to about 20 mg, from about 5 mg to about 15 mg, from about 5 mg to about 10 mg, from about 10 mg to about 25 mg, from about10 mg to about 20 mg, or from about 10 mg to about 15 mg of finerenone in combination with a moderate CYP3A4 inhibitor. In embodiments, the methods comprise administering about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 20.5 mg, about 21 mg, about 21.5 mg, about 22 mg, about 22.5 mg, about 23 mg, about 23.5 mg, about 24 mg, about 24.5 mg, about 25 mg, about 25.5 mg, about 26 mg, about 26.5 mg, about 27 mg, about 27.5 mg, about 28 mg, about 28.5 mg, about 29 mg, about 29.5 mg, or about 30 mg of finerenone in combination with a moderate CYP3A4 inhibitor. In embodiments, the methods comprise administering about 5 mg of finerenone in combination with a moderate CYP3A4 inhibitor. In embodiments, the methods comprise administering about 10 mg of finerenone in combination with a moderate CYP3A4 inhibitor. In embodiments, the methods comprise administering about 20 mg of finerenone in combination with a moderate CYP3A4 inhibitor.

The recommended target daily dose and/or starting daily dose is determined based on the KERENDIA label, dated July 2025, which is incorporated by reference herein in its entirety for all purposes.

In embodiments, provided herein are methods of coadministering finerenone with a strong CYP3A4 inhibitor to a patient in need thereof, comprising administering about 10-60% of a recommended starting daily dose of finerenone recommended for an otherwise similar patient who is not co-administered the strong CYP3A4 inhibitor. In embodiments, the methods comprise administering about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% of the recommended starting daily dose of finerenone. In embodiments, the methods comprise administering about 25% of the recommended starting daily dose of finerenone. In embodiments, the methods comprise administering about 50% of the recommended starting daily dose of finerenone.

In embodiments, the recommended starting daily dose of finerenone is from about 5 mg to about 20 mg. In embodiments, the recommended starting daily dose of finerenone is about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, or about 20 mg.

In embodiments, the method comprises administering a starting daily dose of about 0.5 mg to about 6 mg of finerenone (i.e., from 10-30% of the recommended starting daily dose from about 5 mg to about 20 mg of finerenone) in combination with a strong CYP3A4 inhibitor. In embodiments, the method comprises administering a starting daily dose of about 0.5 mg to about 12 mg of finerenone (i.e., from 10-60% of the recommended starting daily dose from about 5 mg to about 20 mg of finerenone) in combination with a strong CYP3A4 inhibitor. In embodiments, the method comprises administering a starting daily dose of about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, or about 12 mg of finerenone in combination with a strong CYP3A4 inhibitor.

In embodiments, the starting daily dose of finerenone is determined based on serum potassium levels, eGFR, or a combination thereof, prior to initiation of finerenone. In embodiments, if a patient's serum potassium is >5.0 mEq/L, initiation of finerenone is not recommended.

In embodiments, the starting daily dose of finerenone is based on eGFR at initiation of treatment. In embodiments, if a patient's eGFR at initiation of finerenone treatment is ≥60 mL/min/1.73 m², the recommended starting daily dose is 20 mg. In embodiments, when the patient has an eGFR at initiation of ≥60 mL/min/1.73 m² and is also being administered a strong CYP3A4 inhibitor, the method comprises administering about 2 mg to about 12 mg of finerenone (i.e., 10-60% of the recommended starting daily dose of finerenone for a patient that is not administered a CYP3A4 inhibitor). In embodiments, the patient is administered about 2 mg, about 2.5 mg, about 3 mg, about 3.5 mg, about 4 mg, about 4.5 mg, about 5 mg, about 5.5 mg, about 6 mg of finerenone, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, or about 12 mg. In embodiments, the patient is administered about 5 mg of finerenone. In embodiments, the patient is administered about 10 mg of finerenone.

In embodiments, if a patient's eGFR at initiation of finerenone treatment is ≥25 to <60 mL/min/1.73 m², the recommended starting daily dose is 10 mg. In embodiments, when the patient has an eGFR at initiation of ≥25 to <60 mL/min/1.73 m² and the patient is also being administered a strong CYP3A4 inhibitor, the method comprises administering from about 1 mg to about 6 mg of finerenone (i.e., 10-60% of the recommended starting daily dose of finerenone for a patient that is not administered a CYP3A4 inhibitor). In embodiments, the patient is administered about 1 mg, about 1.5 mg, about 2 mg, about 2.5 mg, about 3 mg, about 3.5 mg, about 4 mg, about 4.5 mg, about 5 mg, about 5.5 mg, or about 6 mg of finerenone. In embodiments, the patient is administered about 2.5 mg of finerenone. In embodiments, the patient is administered about 5 mg of finerenone.

In embodiments, if a patient's eGFR is <25 mL/min/1.73 m², the method comprises not administering finerenone.

Table A summarizes the starting finerenone daily dose based on eGFR.

TABLE A
Starting Dose of Finerenone based on eGFR

| eGFR (mL/ | Recommended | Starting Dose with Strong |
|---|---|---|
| min/1.73 m²) | Starting Dose | CYP3A4 Inhibitor |
| ≥60 | 20 mg orally once daily | 5 mg orally once daily |
| ≥25 to <60 | 10 mg orally once daily | 2.5 mg orally once daily |
| <25 | Initiation is not | Initiation is not recommended |
| | recommended | |

In embodiments, provided herein are methods of coadministering finerenone with a strong CYP3A4 inhibitor to a patient in need thereof, comprising administering about 10-40% of a recommended target daily dose of finerenone recommended for an otherwise similar patient who is not co-administered the strong CYP3A4 inhibitor. In embodiments, provided herein are methods of coadministering finerenone with a strong CYP3A4 inhibitor to a patient in need thereof, comprising administering about 10-60% of a recommended target daily dose of finerenone recommended for an otherwise similar patient who is not co-administered the strong CYP3A4 inhibitor. In embodiments, the methods comprise administering about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% of the recommended target daily dose of finerenone in combination with a strong CYP3A4 inhibitor. In embodiments, the methods comprise administering about 25% of the recommended target daily dose of finerenone in combination with a strong CYP3A4 inhibitor. In embodiments, the methods comprise administering about 50% of the recommended target daily dose of finerenone in combination with a strong CYP3A4 inhibitor.

In embodiments, the recommended target daily dose of finerenone is from about 10 mg to about 40 mg of finerenone. In embodiments, the recommended target daily dose of finerenone is about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg. In embodiments, the recommended target daily dose of finerenone is about 10 mg, about 20 mg, or about 40 mg.

In embodiments, the methods provided herein comprise administering from 1 mg to about 3 mg, from about 2 mg to about 6 mg, from about 2.5 mg to about 10 mg, from about 4 mg to about 12 mg, from about 1 mg to about 24 mg, from about 1 mg to about 20 mg, from about 1 mg to about 15 mg, from about 2 mg to about 24 mg, from about 2 mg to about 20 mg, from about 2 mg to about 15 mg, from about 3 mg to about 24 mg, from about 3 mg to about 20 mg, from about 30 mg to about 15 mg, from about 4 mg to about 24 mg, from about 4 mg to about 20 mg, from about 4 mg to about 15 mg, from about 5 mg to about 24 mg, or from about 5 mg to about 20 mg of finerenone in combination with a strong CYP3A4 inhibitor. In embodiments, the methods comprise administering about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 20.5 mg, about 21 mg, about 21.5 mg, about 22 mg, about 22.5 mg, about 23 mg, about 23.5 mg, orabout 24 mg of finerenone in combination with a strong CYP3A4 inhibitor. In embodiments, the methods comprise administering about 2.5 mg of finerenone in combination with a strong CYP3A4 inhibitor. In embodiments, the methods comprise administering about 5 mg of finerenone in combination with a strong CYP3A4 inhibitor. In embodiments, the methods comprise administering about 10 mg of finerenone in combination with a strong CYP3A4 inhibitor. In embodiments, the methods comprise administering about 20 mg of finerenone in combination with a strong CYP3A4 inhibitor.

In embodiments, the recommended target daily dose of finerenone is about 20 mg. In embodiments, when the recommended target daily dose of finerenone is about 20 mg and the patient in need thereof is administered a strong CYP3A4 inhibitor, the patient is administered about 10-30% of the target daily dose (i.e., from about 2 mg to about 6 mg of finerenone). In embodiments, the recommended target daily dose of finerenone is about 20 mg. In embodiments, when the recommended target daily dose of finerenone is about 20 mg and the patient in need thereof is administered a strong CYP3A4 inhibitor, the patient is administered 10-60% of the target daily dose (i.e., from about 2 mg to about 12 mg of finerenone). In embodiments, the patient is administered about 25% of the target daily dose (i.e., about 5 mg of finerenone). In embodiments, the patient is administered about 50% of the target daily dose (i.e., about 10 mg of finerenone).

In embodiments, the recommended target daily dose of finerenone is about 40 mg. In embodiments, when the recommended target daily dose of finerenone is about 40 mg and the patient in need thereof is administered a strong CYP3A4 inhibitor, the patient is administered 10-30% of the recommended target daily dose (i.e., from about 4 mg to about 12 mg of finerenone). In embodiments, the recommended target daily dose of finerenone is about 40 mg. In embodiments, when the recommended target daily dose of finerenone is about 40 mg and the patient in need thereof is administered a strong CYP3A4 inhibitor, the patient is administered 10-60% of the recommended target daily dose (i.e., from about 4 mg to about 24 mg of finerenone). In embodiments, the patient is administered about 25% of the target daily dose (i.e., about 10 mg of finerenone). In embodiments, the patient is administered about 50% of the target daily dose (i.e., about 20 mg of finerenone).

The recommended target daily dose of a patient in need thereof with chronic kidney disease is about 20 mg. In embodiments, when a patient with chronic kidney disease is also being administered a strong CYP3A4 inhibitor, the patient is administered a target daily dose of finerenone of about 2 mg to about 6 mg (i.e., 10-30% of the 20 mg target daily dose the patient would have received in the absence of strong CYP3A4 inhibitor administration). In embodiments, when a patient with chronic kidney disease is also being administered a strong CYP3A4 inhibitor, the patient is administered a target daily dose of finerenone of about 2 mg to about 12 mg (i.e., 10-60% of the 20 mg target daily dose the patient would have received in the absence of strong CYP3A4 inhibitor administration). In embodiments, the patient with chronic kidney disease that is also being administered a strong CYP3A4 inhibitor is administered about 25% of the target daily dose (i.e., about 5 mg of finerenone). In embodiments, the patient with chronic kidney disease that is also being administered a strong CYP3A4 inhibitor is administered about 50% of the target daily dose (i.e., about 10 mg of finerenone). In embodiments, the chronic kidney disease is associated with type 2 diabetes.

The recommended target daily dose of a patient in need thereof with heart failure is about 40 mg if the patient's eGFR at initiation of finerenone treatment is ≥60 mL/min/1.73 m². In embodiments, when a patient with heart failure that has an eGFR at initiation of finerenone treatment of ≥60 mL/min/1.73 m² is also being administered a strong CYP3A4 inhibitor, the patient is administered a target daily dose of finerenone from about 4 mg to about 12 mg (i.e., 10-30% of the 40 mg recommended target daily dose the patient would have received in the absence of strong CYP3A4 inhibitor administration). In embodiments, when a patient with heart failure that has an eGFR at initiation of finerenone treatment of ≥60 mL/min/1.73 m² is also being administered a strong CYP3A4 inhibitor, the patient is administered a target daily dose of finerenone from about 4 mg to about 24 mg (i.e., 10-60% of the 40 mg recommended target daily dose the patient would have received in the absence of strong CYP3A4 inhibitor administration). In embodiments, the patient in need thereof has an LVEF ≥40%. In embodiments, the patient in need thereof is administered about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 20.5 mg, about 21 mg, about 21.5 mg, about 22 mg, about 22.5 mg, about 23 mg, about 23.5 mg, or about 24 mg of finerenone. In embodiments, the patient is administered about 10 mg of finerenone. In embodiments, the patient is administered about 20 mg of finerenone.

The recommended target daily dose of a patient in need thereof with heart failure is about 20 mg if the patient's eGFR at initiation of finerenone treatment is ≥25 to <60 mL/min/1.73 m². In embodiments, when a patient with heart failure that has an eGFR at initiation of finerenone treatment of ≥25 to <60 mL/min/1.73 m² is also being administered a strong CYP3A4 inhibitor, the patient is administered a target daily dose of finerenone from about 2 mg to about 6 mg (i.e., 10-30% of the recommended 20 mg target daily dose the patient would have received in the absence of strong CYP3A4 inhibitor administration). In embodiments, when a patient with heart failure that has an eGFR at initiation of finerenone treatment of ≥25 to <60 mL/min/1.73 m² is also being administered a strong CYP3A4 inhibitor, the patient is administered a target daily dose of finerenone from about 2 mg to about 12 mg (i.e., 10-60% of the recommended 20 mg target daily dose the patient would have received in the absence of strong CYP3A4 inhibitor administration). In embodiments, the patient in need thereof has an LVEF ≥40%. In embodiments, the patient in need thereof is administered about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, or about 12 mg of finerenone. In embodiments, the patient is administered about 5 mg of finerenone. In embodiments, the patient is administered about 10 mg of finerenone.

The KERENDIA label provides instructions for adjusting a patient's target daily dose of finerenone based on current serum potassium concentration. In embodiments, the methods comprise administering a strong CYP3A4 inhibitor and 10-60% of the recommended adjusted dose of finerenone for an otherwise similar patient that is not co-administered the strong CYP3A4 inhibitor based on the patient's current serum potassium concentration. Finerenone dose adjustments for patients that are not receiving strong CYP3A4 inhibitors based on their serum potassium are found in Tables B and C. In embodiments, patients that are receiving CYP3A4 inhibitors in combination with finerenone would receive 10-60% of the adjusted finerenone dose found in Tables B and C, based on their serum potassium.

For example, the FDA-approved label instructs a patient that is not administered a strong CYP3A4 inhibitor and is receiving 10 mg finerenone once daily dose with a serum potassium of ≤4.8 mEq/L four weeks after initiating finerenone treatment to be administered an adjusted dose of 20 mg once daily. In embodiments, if the patient was being administered a strong CYP3A4 inhibitor, the method comprises administering an adjusted dose of about 2 mg to about 12 mg of finerenone once daily (e.g., about 5 mg)). As another example, the FDA-approved label instructs a patient that is not administered a strong CYP3A4 inhibitor and is receiving 20 mg finerenone once daily with a serum potassium of ≤4.8 mEq/L four weeks after treatment with finerenone is initiated, to maintain a 20 mg once daily dose of finerenone. In embodiments, if the patient is administered a strong CYP3A4 inhibitor, the method comprises administering a dose of about 2 mg to about 12 mg of finerenone once daily (e.g., about 5 mg).

In embodiments, the methods described herein further comprise monitoring serum potassium. In embodiments, the methods comprise adjusting the dose of finerenone according to Table B and/or Table C if necessary.

TABLE B
Dose adjustment Based on Current Serum Potassium Concentration and Current Dose (CKD associated with T2DM)

| | | Finerenone Dose | | Finerenone Dose with Strong CYP3A4 Inhibitor | |
|---|---|---|---|---|---|
| | | 10 mg once daily | 20 mg once daily | 2.5 mg once daily | 5 mg once daily |
| Current Serum | ≤4.8 | Recommended dose: Increase | Recommended dose: | Increase the dose to 5 | Maintain 5 mg once daily. |
| Potassium | | the dose to 20 mg once daily | Maintain 20 mg once daily. | mg once daily unless | |
| (mEq/L) | | unless eGFR has decreased by | | eGFR has decreased by | |
| | | more than 30% compared to | | more than 30% | |
| | | previous measurement. If eGFR | | compared to previous | |
| | | has decreased by more than 30% | | measurement. If eGFR | |
| | | compared to previous | | has decreased by more | |
| | | measurement, maintain 10 mg | | than 30% compared to | |
| | | daily dose | | previous measurement, | |
| | | | | maintain 2.5 mg daily | |
| | | | | dose | |
| | >4.8-5.5 | Maintain 10 mg once daily. | Maintain 20 mg once | Maintain 2.5 mg once | Maintain 5 mg once daily. |
| | | | daily. | daily. | |
| | >5.5 | Withhold finerenone. Consider | Withhold finerenone. | Withhold finerenone. | Withhold finerenone. Restart at |
| | | restarting at 10 mg once daily | Restart at 10 mg once | Consider restarting at | 2.5 mg once daily finerenone |
| | | finerenone when serum | daily finerenone when | 2.5 mg once daily | when serum potassium ≤5.0 |
| | | potassium ≤5.0 mEq/L | serum potassium ≤5.0 | finerenone when serum | mEq/L |
| | | | mEq/L | potassium ≤5.0 mEq/L | |

TABLE C
Dose adjustment Based on Current Serum Potassium Concentration, eGFR, and Current Dose (Heart Failure (LVEF ≥ 40%))

| | | | | | Finerenone Dose with Strong | | |
|---|---|---|---|---|---|---|---|
| | | Finerenone Dose | | | CYP3A4 Inhibitor | | |
| | | 10 mg | 20 mg | 40 mg | 2.5 mg | 5 mg | 10 mg |
| | | once daily | once daily | once daily | once daily | once daily | once daily |
| Current Serum | <5.0 | Increase the dose | Maintain 20 mg | Maintain 40 | Increase the dose | Maintain 5 mg once | Maintain 10 |
| Potassium | | to 20 mg once | once daily if eGFR | mg once daily | to 5 mg once | daily if eGFR <60 | mg once daily |
| (mEq/L) | | daily* | <60 mL/min/1.73 | | daily* | mL/min/1.73 m² at | |
| | | | m² at initiation | | | initiation | |
| | | | Otherwise increase | | | Otherwise increase | |
| | | | the dose to 40 mg | | | the dose to 10 mg | |
| | | | once daily* | | | once daily* | |
| | ≥5.0 | Maintain current dose. | | | Maintain current dose. | | |
| | to <5.5 | | | | | | |
| | ≥5.5 | Withhold | Decrease to 10 mg | Decrease to | Withhold | Decrease to 2.5 mg | Decrease to 5 |
| | to <6.0 | finerenone. | once daily | 20 mg once | finerenone. | once daily | mg once daily. |
| | | Restart at 10 mg | | daily. | Restart at 2.5 mg | | |
| | | once daily when | | | once daily when | | |
| | | serum potassium | | | serum potassium | | |
| | | <5.5 mEq/L. | | | <5.5 mEq/L. | | |

| | | |
|---|---|---|
| ≥6.0 | Withhold finerenone. Restart at 10 mg once daily when | Withhold finerenone. Restart at 2.5 mg once daily when |
| | serum potassium <5.5 mEq/L. If repeated serum | serum potassium <5.5 mEq/L. If repeated serum |
| | potassium measurements are ≥5.5 mEq/L, restart | potassium measurements are ≥5.5 mEq/L, restart |
| | finerenone at 10 mg once daily when serum | finerenone at 2.5 mg once daily when serum |
| | potassium <5.0 mEq/L. | potassium <5.0 mEq/L. |

*If eGFR has decreased by more than 30% compared to previous measurement, maintain dose.

In embodiments, the patient in need thereof has chronic kidney disease. In embodiments, the chronic kidney disease is associated with type 2 diabetes. In embodiments, treating chronic kidney disease associated with type 2 diabetes results in one or more of reduced risk of sustained eGFR decline, end-stage kidney disease, cardiovascular death, non-fatal myocardial infarction, and hospitalization for heart failure.

In embodiments, the methods described herein comprising coadministering finerenone and a strong CYP3A4 inhibitor reduce the risk of a comorbidity of chronic kidney disease. In embodiments, the chronic kidney disease and/or comorbidity of chronic kidney disease comprises one or more of diabetes mellitus, hypertension, cardiovascular disease, obesity, anemia, a disruption in calcium, phosphorus, sustained estimated glomerular filtration rate (eGFR) decline, end stage kidney disease, cardiovascular death, non-fatal myocardial infarction, hospitalization for heart failure, vitamin D metabolism, malnutrition, depression, anxiety, dyslipidemia, peripheral artery disease (PAD), or infection. In embodiments, chronic kidney disease and the comorbidity of chronic kidney disease comprises sustained estimated glomerular filtration rate (eGFR) decline, end stage kidney disease, cardiovascular death, non-fatal myocardial infarction, or hospitalization.

In embodiments, the chronic kidney disease is associated with type 1 diabetes. In embodiments, the patient in need thereof with chronic kidney disease associated with type 1 diabetes is albuminuric. In embodiments, the patient in need thereof with chronic kidney disease associated with type 1 diabetes is administered an angiotensin-converting enzyme (ACE) inhibitor, an angiotensin receptor blocker (ARB), or a combination thereof. Non-limiting examples of ACE inhibitors include lisinopril, enalapril, ramipril, benazepril, captopril, fosinopril, perindopril, quinapril, and trandolapril. Non-limiting examples of ARB include losartan, valsartan, irbesartan, candesartan, olmesartan, telmisartan, azilsartan, and eprosartan.

In embodiments, the chronic kidney disease is non-diabetic chronic kidney disease. Non-diabetic chronic kidney disease is chronic kidney disease that is not caused by diabetes. In embodiments, the chronic kidney disease is proteinuric chronic kidney disease. In embodiments, the chronic kidney disease is broad proteinuric chronic kidney disease. In embodiments, a patient with proteinuric chronic kidney disease has proteinuria or broad proteinuria.

In embodiments, the patient in need thereof has pediatric chronic kidney disease. In embodiments, the patient in need thereof with pediatric chronic kidney disease is from 6 months old to about 18 years old. In embodiments, the patient is receiving an ACE inhibitor and/or ARB, as described herein. In embodiments, the patient treated according to the methods described herein has pediatric chronic kidney disease and/or proteinuria. In embodiments, the patient with pediatric chronic kidney disease and/or proteinuria is administered an ACE inhibitor, a ARB, or a combination thereof.

In embodiments, the patient treated according to the methods described herein has pediatric heart failure, left ventricular systolic dysfunction, or a combination thereof.

In embodiments, the patient in need thereof has heart failure. In embodiments, the patient has heart failure with mid-range ejection fraction (LVEF from 41% to 49%). In embodiments, the patient has heart failure with preserved ejection fraction (LVEF ≥50%). In embodiments, the patient with heart failure has an LVEF ≥40%. In embodiments, treating heart disease according to the methods described herein reduces one or more of cardiovascular death, hospitalization for heart failure, and the chance of urgent heart failure visits. Non-limiting methods for measuring LVEF include non-invasive methods (echocardiography, MRI, CT, gated equilibrium radionuclide angiography (MUGA scan), and gated myocardial perfusion imaging with either single-photon emission computed tomography (SPECT) or positron emission tomography (PET)) and invasive methods methods. Ejection fraction is then calculated from volume estimates based on this imaging. The following references describe how to calculate LVEF and are incorporated by reference herein in their entirety for all purposes: Foley et al., Measuring Left Ventricular Ejection Fraction—Techniques and Potential Pitfalls, European Cardiology 2012; 8 (2): 108-14; Lang et al. J Am Soc Echocardiogr 2015; 28:1-39.

In embodiments, the patient has an LVEF ≥40%. In embodiments, the patient has an LVEF of <50%. In embodiments, the patient has an LVEF of ≥50% to <60%. In embodiments, the patient has an LVEF of ≥60%. In embodiments, the patient's LVEF is ≥40%, ≥50%, ≥60%, ≥70%, ≥80%, or ≥90%. In embodiments, the patient's LVEF is <50%, <40%, <30%, <20%, or <10%. In embodiments, the patient has an LVEF from 10% to about 80%, from about 10% to about 30%, from about 40% to about 80%, or from about 40% to about 90%.

In embodiments, the methods described herein comprising coadministering finerenone and a strong CYP3A4 inhibitor to reduce the risk of a comorbidity of heart failure. In embodiments, the comorbidity of heart failure comprises hypertension, diabetes mellitus, obesity, chronic kidney disease, coronary artery disease, atrial fibrillation, chronic obstructive pulmonary disease (COPD), anemia, stroke, anxiety, depression, sustained estimated glomerular filtration rate (eGFR) decline, end stage kidney disease, cardiovascular death, non-fatal myocardial infarction, hospitalization, cardiovascular death, urgent heart failure visit, or a combination thereof. In embodiments, the comorbidity is cardiovascular death, hospitalization or urgent heart failure visit.

In embodiments, the patient treated according to the method described herein has IgA nephropathy (IgAN). In embodiments, the patient with IgA nephropathy is an adult. In embodiments, the patient with IgA nephropathy has primary IgA nephropathy. In embodiments, the patient with IgA nephropathy has proteinuria.

In embodiments, the patient treated according to the method described herein has non-diabetic CKD, hypertensive CKD, focal segmental glomerulosclerosis (FSGS), membraneous nephropathy, mesangioproliferative nephropathy, alport nephropathy, or a combination thereof.

In embodiments, the patient treated according to the method described herein is also administered a sodium-glucose cotransporter-2 inhibitor (SGLT2 inhibitor). Non-limiting examples of SGLT2 inhibitors include canagliflozin, dapagliflozin, empagliflozin, ertugliflozin, bexagliflozin, and sotagliflozin.

In embodiments, the patient treated according to the method described herein is also administered a GLP-1 receptor agonist. Non-limiting examples of GLP-1 receptor agonists include semaglutide, tirzepatide, liraglutide, dulaglutide, exenatide, and lixisenatide.

In embodiments, the patient treated according to the methods described herein is also administered a statin. Non-limiting examples of statins include atorvastatin, rosuvastatin, simvastatin, lovastatin, pravastatin, fluvastatin, and pitavastatin.

In embodiments, the patient treated according to the methods described herein is also administered a beta-blocker. Non-limiting examples of beta-blockers include atenolol, metoprolol, propranolol, carvedilol, labetalol, nebivolol, bisoprolol, timolol, acebutolol, betaxolol, esmolol, nadolol, penbutolol, and sotalol.

In embodiments, the patient treated according to the methods described herein is also administered a calcium channel blocker. Non-limiting examples of calcium channel blockers include amlodipine, diltiazem, nifedipine, verapamil, felodipine, isradipine, nicardipine, nimodipine, and nisoldipine.

In embodiments, the patient treated according to the method described herein is also administered an immunosuppressive therapy. In embodiments, the immunosuppressive therapy is a calcineurin inhibitor, an antimetabolite, a corticosteroid, an mTOR inhibitor, a monoclonal antibody, an antibody drug conjugate, a JAK inhibitor, hydroxychloroquine, thalidomide, sulfasalazine, cyclophosphamide, or an inosine monophosphate dehydrogenase inhibitor. In embodiments, the calcineurin inhibitor is cyclosporine, tacrolimus, voclosporin, or a combination thereof. In embodiments, the antimetabolite is mycophenolate mofetil, mycophenolic acid, azathioprine, methotrexate, leflunomide, mizorbine, or a combination thereof. In embodiments, the mTOR inhibitor is sirolimus, everolimus, or a combination thereof. In embodiments, the immunosuppressive therapy is basiliximab, antithymocyte globulin, rituximab, abatacept, belatacept, belimumab, tocilizumab, infliximab, adalimumab, ustekinumab, etanercept, vedolizumab, alemtuzumab, muromonab-CD3, natalizumab, golimumab, certolizumab, secukinumab, ixekizumab, brodalumab, guselkumab, mepolizumab, reslizumab, benralizumab, dupilumab, omalizumab. In embodiments, the JAK inhibitor is tofacitinib, ruxolitinib, or a combination thereof. In embodiments, the corticosteroid is prednisone, prednisolone, methylprednisolone, dexamethasone, hydrocortisone, or a combination thereof.

In embodiments, the patient treated according to the method described herein is also administered a steroid. In embodiments, the steroid is prednisone, cortisone, hydrocortisone, methylprednisolone, triamcinolone, dexamethasone, betamethasone, budesonide, fluticasone, clobetasol, fludrocortisone, beclomethasone, mometasone, or a combination thereof.

In embodiments, the patient treated according to the method described herein is also administered allopurinol.

In embodiments, the patient treated according to the method described herein has one or more of diabetic kidney disease, anemia, hyperkalemia, frailty, atherosclerotic cardiovascular disease, cardiovascular-kidney-metabolic syndrome, a cardiovascular disease, chronic kidney disease, a metabolic condition, or a worsening heart failure event.

In embodiments, the patient treated according to the method described herein is 18 to 65 years old. In embodiments, the patient is 65 years old or older. In embodiments, the patient is from 6 months to 18 years old. In embodiments, the patient is less than 6 months old.

In embodiments, the patient treated according to the method described herein had a recent heart failure event.

In embodiments, the patient treated according to the method described herein is obese.

In some embodiments, a patient with a BMI of at least about 30 to at least about 34.9, at least about 30.5 to at least about 34, at least about 31 to at least about 33.5, at least about 31.5 to at least about 33, or at least about 32 to at least about 32.5, inclusive of all ranges and subranges therebetween can be considered obese. In some embodiments, a patient with a BMI of at least about 35 to at least about 39.9, at least about 35.5 to at least about 39, at least about 36 to at least about 38.5, at least about 36.5 to at least about 38, or at least about 37 to at least about 37.5, inclusive of all ranges and subranges therebetween, and any BMI described herein, can be considered obese.

In some embodiments, the class of patients treated by the methods of the present disclosure have a body mass index (BMI; expressed in units of kg/m² unless otherwise specified) of less than about 25, e.g., about 24.5, about 24, about 23.5, about 23, about 22.5, about 22, about 21.5, about 21, about 20.5, about 20, about 19.5, about 19, or about 18.5 or less, inclusive of all values and ranges therebetween.

In some embodiments, the class of patients treated by the methods of the present disclosure have a body mass index (BMI; expressed in units of kg/m² unless otherwise specified) of at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, at least about 100, at least about 101, at least about 102, at least about 103, at least about 104, at least about 105, at least about 106, at least about 107, at least about 108, at least about 109, at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, at least about 141, at least about 142, at least about 143, at least about 144, at least about 145, at least about 146, at least about 147, at least about 148, at least about 149, at least about 150, at least about 151, at least about 152, at least about 153, at least about 154, at least about 155, at least about 156, at least about 157, at least about 158, at least about 159, at least about 160, at least about 161, at least about 162, at least about 163, at least about 164, at least about 165, at least about 166, at least about 167, at least about 168, at least about 169, at least about 170, at least about 171, at least about 172, at least about 173, at least about 174, at least about 175, at least about 176, at least about 177, at least about 178, at least about 179, at least about 180, at least about 181, at least about 182, at least about 183, at least about 184, at least about 185, at least about 186, at least about 187, at least about 188, at least about 189, at least about 190, at least about 191, at least about 192, at least about 193, at least about 194, at least about 195, at least about 195, at least about 196, at least about 197, at least about 198, at least about 199, at least about 200, at least about 201, at least about 202, at least about 203, at least about 204, at least about 205, at least about 206, at least about 207, at least about 208, at least about 209, or at least about 210, inclusive of all ranges and subranges therebetween, and any BMI described herein. In one embodiment, the patient has a body mass index (BMI) of at least about 35. In another embodiment, the patient has a body mass index (BMI) of at least about 40. In another embodiment, the patient has a body mass index (BMI) of at least 50.

In some embodiments, a patient treated according to the methods of the present invention has a BMI of at least about 25 to at least about 29.9, at least about 25.5 to at least about 29, at least about 26 to at least about 28.5, at least about 26.5 to at least about 28, or at least about 27 to at least about 27.5, inclusive of all ranges and subranges therebetween, and can be termed overweight or pre-obese. In some embodiments, a patient with a BMI of at least about 30 to at least about 34.9, at least about 30.5 to at least about 34, at least about 31 to at least about 33.5, at least about 31.5 to at least about 33, or at least about 32 to at least about 32.5, inclusive of all ranges and subranges therebetween can be considered obese. In some embodiments, a patient with a BMI of at least about 35 to at least about 39.9, at least about 35.5 to at least about 39, at least about 36 to at least about 38.5, at least about 36.5 to at least about 38, or at least about 37 to at least about 37.5, inclusive of all ranges and subranges therebetween, and any BMI described herein, can be considered obese. In other embodiments, a patient treated by the methods of the present disclosure has a BMI of at least about 35 or more, 40 or more, 50 or more, 60 or more, 70 or more, 80 or more, 90 or more, 100 or more, 110 or more, 120 or more, 130 or more, 140 or more, 150 or more, 160 or more, 170 or more, 180 or more, 190 or more, 200 or more, or 210 or more, inclusive of all ranges and subranges therebetween.

In some embodiments, the patient treated according to the methods of the present disclosure is a child or an adolescent with a BMI of at least about the 85^{th} percentile to at least about 95^{th} percentile, at least about the 86^{th} percentile to at least about 94^{th} percentile, at least about the 87^{th} percentile to at least about 93^{th} percentile, at least about the 88^{th} percentile to at least about 92^{th} percentile, at least about the 89^{th} percentile to at least about 90^{th} percentile, inclusive of all ranges and subranges therebetween, can be considered overweight or pre-obese. In some embodiments, the patient is a patient with a BMI of at least about the 95^{th} percentile, at least about 96^{th} percentile, at least about the 97^{th} percentile, at least about 98^{th} percentile, at least about 99^{th} percentile, or at least about 100^{th} percentile, inclusive of all ranges and subranges therebetween, and any BMI percentile described herein, and can be considered obese. In one embodiment, the patient is about 5 to about 19 years old or about 7 to about 18 years old.

In some embodiments, the patient treated according to the methods of the present disclosure is a female patient in the first trimester through third trimester of a pregnancy and has a BMI of at least 25 to at least about 29.9, at least about 25.5 to at least about 29, at least about 26 to at least about 28.5, at least about 26.5 to at least about 28, or at least about 27 to at least about 27.5, inclusive of all ranges and subranges therebetween, and can be considered overweight or pre-obese. In some embodiments, the patient is a female patient in the first trimester through third trimester of a pregnancy and has a BMI of at least about 30 to at least about 34.9, at least about 30.5 to at least about 34, at least about 31 to at least about 33.5, at least about 31.5 to at least about 33, or at least about 32 to at least about 32.5, inclusive of all ranges and subranges therebetween, and can be considered obese. In some embodiments, the patent treated according to the methods of the present invention is a female patient in the first trimester through third trimester of a pregnancy and has a BMI of at least about 35 to at least about 39.9, at least about 35.5 to at least about 39, at least about 36 to at least about 38.5, at least about 36.5 to at least about 38, at least about 37 to at least about 37.5, inclusive of all ranges and subranges therebetween, and can be considered severely obese.

In some embodiments, methods of calculating BMI may include, but are not limited to body weight in kilogram/(height in meters) 2, body weight in pounds/(height in inches) 2]×703, and the like.

In some embodiments, the patient treated according to the methods of the present disclosure can alternatively be described as having a % ideal body weight (% IBW) of at least about 110%, at least about 111%, at least about 112%, at least about 113%, at least about 114%, at least about 115%, at least about 116%, at least about 117%, at least about 118%, at least about 119%, at least about 120%, at least about 121%, at least about 122%, at least about 123%, at least about 124%, at least about 125%, at least about 126%, at least about 127%, at least about 128%, at least about 129%, at least about 130%, at least about 131%, at least about 132%, at least about 133%, at least about 134%, at least about 135%, at least about 136%, at least about 137%, at least about 138%, at least about 139%, at least about 140%, at least about 141%, at least about 142%, at least about 143%, at least about 144%, at least about 145%, at least about 146%, at least about 147%, at least about 148%, at least about 149%, at least about 150%, at least about 151%, at least about 152%, at least about 153%, at least about 154%, at least about 155%, at least about 156%, at least about 157%, at least about 158%, at least about 159%, at least about 160%, at least about 161%, at least about 162%, at least about 163%, at least about 164%, at least about 165%, at least about 166%, at least about 167%, at least about 168%, at least about 169%, at least about 170%, at least about 171%, at least about 172%, at least about 173%, at least about 174%, at least about 175%, at least about 176%, at least about 177%, at least about 178%, at least about 179%, at least about 180%, at least about 181%, at least about 182%, at least about 183%, at least about 184%, at least about 185%, at least about 186%, at least about 187%, at least about 188%, at least about 189%, at least about 190%, at least about 191%, at least about 192%, at least about 193%, at least about 194%, at least about 195%, at least about 196%, at least about 197%, at least about 198%, at least about 199%, at least about 200%, at least about 201%, at least about 202%, at least about 203%, at least about 204%, at least about 205%, at least about 206%, at least about 207%, at least about 208%, at least about 209%, at least about 210%, at least about 211%, at least about 212%, at least about 213%, at least about 214%, at least about 215%, at least about 216%, at least about 217%, at least about 218%, at least about 219%, at least about 220%, at least about 221%, at least about 222%, at least about 223%, at least about 224%, at least about 225%, at least about 226%, at least about 227%, at least about 228%, at least about 229%, at least about 230%, at least about 231%, at least about 232%, at least about 233%, at least about 234%, at least about 235%, at least about 236%, at least about 237%, at least about 238%, at least about 239%, at least about 240%, at least about 241%, at least about 242%, at least about 243%, at least about 244%, at least about 245%, at least about 246%, at least about 247%, at least about 248%, at least about 249%, at least about 250%, at least about 251%, at least about 252%, at least about 253%, at least about 254%, at least about 255%, at least about 256%, at least about 257%, at least about 258%, at least about 259%, at least about 260%, at least about 261%, at least about 262%, at least about 263%, at least about 264%, at least about 265%, at least about 266%, at least about 267%, at least about 268%, at least about 269%, at least about 270%, at least about 271%, at least about 272%, at least about 273%, at least about 274%, at least about 275%, at least about 276%, at least about 277%, at least about 278%, at least about 279%, or at least about 280%, inclusive of all ranges and subranges therebetween, and any % ideal body weight described herein. In one embodiment, the patient has % ideal body weight (IBW) of at least about 150%. In one embodiment, the patient has % ideal body weight (IBW) of at least about 250%. In other embodiments, the patient has % IBW of at least 150% and can be considered obese.

In some embodiments, the patient treated according to the present disclosure can alternatively be described as having a waist size or waist circumference greater than about 32, greater than about 33, greater than about 34, greater than about 35 inches, greater than about 36, greater than about 37, greater than about 38, greater than about 39, greater than about 40, greater than about 41, greater than about 42, greater than about 43, greater than about 44, greater than about 45, greater than about 46, greater than about 47, greater than about 48, greater than about 49, greater than about 50, greater than about 51, greater than about 52, greater than about 53, greater than about 54, greater than about 55, greater than about 56, greater than about 57, greater than about 58, greater than about 59, greater than about 60 inches, greater than about 61 inches, greater than about 62 inches, greater than about 63 inches, greater than about 64 inches, greater than about 65 inches, inclusive of all ranges and subranges therebetween, and any waist size or circumference described herein. In one embodiment, a patient having a waist size or waist circumference of about 42 inches can be considered obese. In another embodiment, the patient has waist size or waist circumference greater than about 48 inches. In other embodiment, the patient has waist or waist circumference of at least 42 inches.

In some embodiments, the patient treated according to the methods of the present disclosure can alternatively be described as having a % body fat greater than about 20%, greater than about 21%, greater than about 22%, greater than about 23%, greater than about 24%, greater than about 25%, greater than about 26%, greater than about 27%, greater than about 28%, greater than about 29%, greater than about 30%, greater than about 31%, greater than about 32%, greater than about 33%, greater than about 34%, greater than about 35%, greater than about 36%, greater than about 37%, greater than about 38%, greater than about 39%, greater than about 40%, greater than about 41%, greater than about 42%, greater than about 43%, greater than about 44%, greater than about 45%, greater than about 46%, greater than about 47%, greater than about 48%, greater than about 49%, or greater than about 50%, inclusive of all ranges and subranges therebetween, and any % body fat described herein. In one embodiment, the patient has a % body fat greater than about 40%. In one embodiment, the patient has a % body fat of at least about 50%. In another embodiment, a patient having a % body fat greater than about 40% can be considered obese. In some embodiments, methods of calculating % body fat can include, but are not limited to total body fat expressed as a percentage of total body weight. Other standards for obesity can be used. For example, the American Council on Exercise suggests that an “average” percentage of body fat for women is about 25-31%, and for men, about 18-24%, and for obese women, about 32% and higher, and obese men, about 25% and higher.

In other embodiments, the patient can alternatively be described as having a % android body fat greater than about 30%, greater than about 31%, greater than about 32%, greater than about 33%, greater than about 34%, greater than about 35%, greater than about 36%, greater than about 37%, greater than about 38%, greater than about 39%, greater than about 40%, greater than about 41%, greater than about 42%, greater than about 43%, greater than about 44%, greater than about 45%, greater than about 46%, greater than about 47%, greater than about 48%, greater than about 49%, greater than about 50%, greater than about 51%, greater than about 52%, greater than about 53%, greater than about 54%, greater than about 55%, greater than about 56%, greater than about 57%, greater than about 58%, greater than about 59%, greater than about 60%, greater than about 61%, greater than about 62%, greater than about 63%, greater than about 64%, greater than about 65%, greater than about 66%, greater than about 67%, greater than about 68%, greater than about 69%, greater than about 70%, greater than about 71%, greater than about 72%, greater than about 73%, greater than about 74%, greater than about 75%, greater than about 76%, greater than about 77%, greater than about 78%, greater than about 79%, or greater than about 80%, inclusive of all ranges and subranges therebetween, and any % android body fat described herein. In one embodiment, a patient having a % android body fat greater than about 40% can be considered obese. In one embodiment, a patient having a % android body fat greater than about 50% can be considered obese.

In other embodiments, the patient can alternatively be described as having a % android body fat of at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, or at least about 80%, inclusive of all ranges and subranges therebetween, and % android body fat described herein. In one embodiment, the patient has % android body fat of at least about 50%.

In other embodiments, the patient can alternatively be described as having a % gynoid body fat greater than about 30%, greater than about 31%, greater than about 32%, greater than about 33%, greater than about 34%, greater than about 35%, greater than about 36%, greater than about 37%, greater than about 38%, greater than about 39%, greater than about 40%, greater than about 41%, greater than about 42%, greater than about 43%, greater than about 44%, greater than about 45%, greater than about 46%, greater than about 47%, greater than about 48%, greater than about 49%, greater than about 50%, greater than about 51%, greater than about 52%, greater than about 53%, greater than about 54%, greater than about 55%, greater than about 56%, greater than about 57%, greater than about 58%, greater than about 59%, greater than about 60%, greater than about 61%, greater than about 62%, greater than about 63%, greater than about 64%, greater than about 65%, greater than about 66%, greater than about 67%, greater than about 68%, greater than about 69%, greater than about 70%, greater than about 71%, greater than about 72%, greater than about 73%, greater than about 74%, greater than about 75%, greater than about 76%, greater than about 77%, greater than about 78%, greater than about 79%, or greater than about 80%, inclusive of all ranges and subranges therebetween, and any % gynoid body fat described herein. In one embodiment, a patient having a % gynoid body fat greater than about 40% can be considered obese. In one embodiment, a patient having a % gynoid body fat greater than about 50% can be considered obese.

In other embodiments, the patient can alternatively be described as having a total body fat content greater than about 30 kg, greater than about 31 kg, greater than about 32 kg, greater than about 33 kg, greater than about 34 kg, greater than about 35 kg, greater than about 36 kg, greater than about 37 kg, greater than about 38 kg, greater than about 39 kg, greater than about 40 kg, greater than about 41 kg, greater than about 42 kg, greater than about 43 kg, greater than about 44 kg, greater than about 45 kg, greater than about 46 kg, greater than about 47 kg, greater than about 48 kg, greater than about 49 kg, greater than about 50 kg, greater than about 51 kg, greater than about 52 kg, greater than about 53 kg, greater than about 54 kg, greater than about 55 kg, greater than about 56 kg, greater than about 57 kg, greater than about 58 kg, greater than about 59 kg, greater than about 60 kg, greater than about 61 kg, greater than about 62 kg, greater than about 63 kg, greater than about 64 kg, greater than about 65 kg, greater than about 66 kg, greater than about 67 kg, greater than about 68 kg, greater than about 69 kg, greater than about 70 kg, greater than about 71 kg, greater than about 72 kg, greater than about 73 kg, greater than about 74 kg, greater than about 75 kg, greater than about 76 kg, greater than about 77 kg, greater than about 78 kg, greater than about 79 kg, greater than about 80 kg, greater than about 81 kg, greater than about 82 kg, greater than about 83 kg, greater than about 84 kg, greater than about 85 kg, greater than about 86 kg, greater than about 87 kg, greater than about 88 kg, greater than about 89 kg, greater than about 90 kg, greater than about 91 kg, greater than about 92 kg, greater than about 93 kg, greater than about 94 kg, greater than about 95 kg, greater than about 96 kg, greater than about 97 kg, greater than about 98 kg, greater than about 99 kg, greater than about 100 kg, at least 101 kg, at least 102 kg, at least 103 kg, at least 104 kg, at least 105 kg, at least 106 kg, at least 107 kg, at least 108 kg, at least 109 kg, or at least 110 kg, inclusive of all ranges and subranges therebetween, and any total body fat described herein. In one embodiment, a patient having total body fat greater than about 40 kg can be considered obese. In one embodiment, a patient having total body fat greater than about 50 kg can be considered obese.

In other embodiments, the obesity status of patients treated with the methods of the present disclosure can be measured by waist-to-hip ratio. In other embodiments, the obesity status of patients can be measured by skinfold thickness. In other embodiments, the obesity status of patients can be measured by bioelectric impedance. In other embodiments, the obesity status of patients can be measured by underwater weighing or densitometry. In other embodiments, the obesity status of patients can be measured by air-displacement plethysmography. In other embodiments, the obesity status of patients can be measured by dilution method or hydrometry. In other embodiments, the obesity status of patients can be measured by dual energy X-ray absorptiometry. In other embodiments, the obesity status of patients can be measured by computerized tomography and magnetic resonance imaging. In some embodiments, the obesity status can be defined by, but is not limited to adopting the clinical standards, conventional standards, and/or the standards published by the World Health Organization and Center of Disease Control (both of which are herein incorporated by reference in their entireties for all purposes) when using the methods described herein. For example, the WHO defines an obese person as a person with a BMI of 30 or more, an overweight person is one with a BMI equal to or more than 25 (to less than 30). Similarly, the CDC defines normal as a BMI of 18.5 to less than 25, 25.0 to less than 30 as overweight, and 30.0 or higher as obese. The CDC further subdivides obesity into 3 classes: Class 1, a BMI of 30 to less than 35; Class 2, a BMI of 35 to less than 40; and Class 3, as a BMI of 40 or higher. The CDC sometimes refers to Class 3 obesity as “extreme” or “severe” obesity.

In some embodiments, the patient treated by the methods of the present disclosure can be characterized by two or more of the physiological characteristics described herein. For example the patient can have a BMI of at least about 35 and can have a % IBW of at least 150%. In some embodiments, the patient can have a BMI of at least about 35 and can have a waist size greater than about 42 inches. In some embodiments, the patient can have a BMI of at least about 35 and can have a % body fat greater than about 40%. In some embodiments, the patient can have a BMI of at least about 35 and can have a % android body fat greater than about 40%. In some embodiments, the patient can have a BMI of at least about 35 and can have a % gynoid body fat greater than about 40%. In some embodiments, the patient can have a BMI of at least about 35 and can have total body fat greater than about 40 kg. In various other embodiments, the patient can have any combination of two or more of any of the specific physiological parameters described herein.

In some embodiments, the patient can have three or more of the physiological parameters described herein, for example a BMI of at least about 35, a % IBW of at least 150%, and waist size greater than about 42 inches. In some embodiments, the patient can have a BMI of at least about 35, a % IBW of at least 150%, and a % body fat greater than about 40%. In some embodiments, the patient can have a BMI of at least about 35, a % IBW of at least 150%, and a % android body fat greater than about 40%. In some embodiments, the patient can have a BMI of at least about 35, a % IBW of at least 150%, and a % gynoid body fat greater than about 40%. In some embodiments, the patient can have a BMI of at least about 35, a % IBW of at least 150%, and total body fat greater than about 40 kg. In various other embodiments, the patient can have any combination of three or more of any of the specific physiological parameters described herein.

In some embodiments, the patient can have four or more of the physiological parameters described herein, for example the patient can have a BMI of at least about 35, a % IBW of at least 150%, waist size greater than about 42 inches, and a % body fat greater than about 40%. In some embodiments, the patient can have a BMI of at least about 35, a % IBW of at least 150%, waist size greater than about 42 inches, and a % android body fat greater than about 40%. In some embodiments, the patient can have a BMI of at least about 35, a % IBW of at least 150%, waist size greater than about 42 inches, and a % gynoid body fat greater than about 40%. In some embodiments, the patient can have a BMI of at least about 35, a % IBW of at least 150%, a waist size greater than about 42 inches, and total body fat greater than about 43 kg. In some embodiments, the patient can have a BMI of at least about 35, a % IBW of at least 150%, a waist size greater than about 42 inches, a % body fat greater than about 40%, and a % android body fat greater than about 40%. In some embodiments, the patient can have a BMI of at least about 35, a % IBW of at least 150%, a waist size greater than about 42 inches, a % body fat greater than about 40%, and a % gynoid body fat greater than about 40%. In some embodiments, the patient can have a BMI of at least about 35, a % IBW of at least 150%, a waist size greater than about 42 inches, a % body fat greater than about 40%, and total body fat greater than about 40 kg. In some embodiments, the patient can have a BMI of at least about 35, a % IBW of at least 150%, a waist size greater than about 42 inches, a % body fat greater than about 40%, a % android body fat greater than about 40%, in % gynoid body fat greater than about 40%, and total body fat greater than about 40 kg. In one embodiment, the patient who has a BMI of at least about 35, in % IBW of at least 150%, a waist size greater than about 42 inches, and a % body fat greater than about 40%, a % android body fat greater than about 40%, a % gynoid body fat greater than about 40%, and total body fat greater than about 40 kg. In various other embodiments, the patient can have any combination of any or all of the specific physiological parameters described herein.

In some embodiments, the patient can have a waist size greater than about 42 inches, a % body fat greater than about 40%, and a % android body fat greater than about 40%. In some embodiments, the patient can have a waist size greater than about 42 inches, a % body fat greater than about 40%, and a % gynoid body fat greater than about 40%. In some embodiments, the patient can have a waist size greater than about 42 inches, a % body fat greater than about 40%, and total body fat greater than about 40 kg.

In some embodiments, the patient can have a % body fat greater than about 40%, a % android body fat greater than about 40%, and a % gynoid body fat greater than about 40%. In some embodiments, the patient can have a % body fat greater than about 40%, a % android body fat greater than about 40%, and total body fat greater than about 40 kg. In some embodiments, the patient can have a % body fat greater than about 40%, a % gynoid body fat greater than about 40%, and total body fat greater than about 40 kg. In some embodiments, a % android body fat greater than about 40%, and a % gynoid body fat greater than about 40%, and total body fat greater than about 43 kg. In some embodiments, the patient can have any combinations of obesity characteristics described herein.

In embodiments, the patient in need thereof is administered finerenone in combination with one or more of a diuretic, a RAS inhibitor, or a SGLT2 inhibitor. In embodiments, the patient in need thereof is administered a drug that blocks the renin-angiotensin system. In embodiments, the drug that blocks the renin-angiotensin system is an ACE inhibitor, an ARB, or a direct renin inhibitor. Non-limiting examples of ACE inhibitors and ARB are provided throughout this application. Non-limiting examples of direct renin inhibitors include aliskiren, ditekiren, and remikren.

In embodiments, the disclosure also provides for methods of increasing the finerenone dose after the patient has stopped receiving treatment with the strong CYP3A4 inhibitor. In embodiments, the dose of finerenone is increased to the dose of finerenone recommended for the patient on the FDA label, for example, to the target daily daily dose. In embodiments, after the patient has stopped receiving the strong CYP3A4 inhibitor, the methods comprising delaying administering the target daily dose of finerenone. In embodiments, the length of time between stopping strong CYP3A4 inhibitor administration and administering the target daily dose of finerenone depends on the half life of the CYP3A4 inhibitor. In embodiments, after the patient has stopped receiving the strong CYP3A4 inhibitor, the methods comprising delaying administering the target daily dose of finerenone for about 1-31 days, about 1-28 days, about 1-25 days, about 1-24 days, about 1-21 days, about 1-18 days, about 1-15 days, about 1-12 days, about 1-9 days, or about 1-7 days, and then administering the target daily dose of finerenone. In embodiments, after the patient has stopped receiving the strong CYP3A4 inhibitor, the methods comprising delaying administering the target daily dose of finerenone for about 1-31 days, and then administering the target daily dose of finerenone. In embodiments, after the patient has stopped receiving the strong CYP3A4 inhibitor, the methods comprising delaying administering the target daily dose of finerenone for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days, and then administering the target daily dose of finerenone. In embodiments, after the patient has stopped receiving the strong CYP3A4 inhibitor, the methods comprise delaying administering the target daily dose of finerenone for about 2-30, about 2-27, about 2-24, about 2-21, about 2-18, about 2-15, about 2-12, about 2-9, or about 2-7 half-lives of the strong CYP3A4 inhibitor, and then administering the target daily dose of finerenone. In embodiments, after the patient has stopped receiving the strong CYP3A4 inhibitor, the methods comprising delaying administering the target daily dose of finerenone for about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 half-lives of the strong CYP3A4 inhibitor, and then administering the target daily dose of finerenone. In embodiments, after the patient has stopped receiving the strong CYP3A4 inhibitor, the methods comprise delaying administering the target daily dose of finerenone for about 2-30 half-lives of the strong CYP3A4 inhibitor, and then administering the target daily dose of finerenone. In embodiments, after the patient has stopped receiving the strong CYP3A4 inhibitor, the methods comprise delaying administering the target daily dose of finerenone for about 3-5 half-lives of the strong CYP3A4 inhibitor, and then administering the target daily dose of finerenone. The half lives of strong CYP3A4 inhibitors can be found on the Food and Drug Administration's Prescribing Information insert. For example, the Prescribing Information for itraconazole (TOLSURA), revised October 2024, states that the terminal half life of itraconazole following repeated dose administration of TOLSURA ranges between 34 to 42 hours under fed conditions.

In some embodiments, after the patient has stopped receiving the strong CYP3A4 inhibitor, the methods comprise titrating to the target daily dose of finerenone. In embodiments, the methods comprise continuing to administer about 10% to about 30% of the target daily dose of finerenone for a first period of time, titrating up to a higher dose that is less the target daily dose of finerenone, and administering the higher dose of a second period of time, then administering the target daily dose of finerenone. In embodiments, after the patient has stopped receiving the strong CYP3A4 inhibitor, the methods comprise administering a first dose of finerenone comprising from about 10% to about 30% of the target daily dose of finerenone for about 1 to about 31 days after stopping the strong CYP3A4 inhibitor, subsequently administering a second dose (e.g., about 3 to about 31 days after stopping the CYP3A4 inhibitor) of finerenone that is higher than the first dose of finerenone but lower than the target daily dose of finerenone, and subsequently administering the target daily dose of finerenone. In embodiments, the first dose of finerenone comprises from about 10% to about 30% (e.g., about 10%, about 15%, about 20%, about 25%, or about 30%) of the target daily dose of finerenone. In embodiments, the second dose of finerenone comprises from about 15% to about 85%, from about 15% to about 75%, from about 15% to about 65%, from about 15% to about 55%, or from about 15% to about 45% of the target daily dose of finerenone. In embodiments, the first and/or second dose is administered for about 1 to about 31 days after stopping the strong CYP3A4 inhibitor. In embodiments, the first and/or second dose is administered for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days. In embodiments, after the patient has stopped receiving the CYP3A4 inhibitor, the methods comprise administering a first dose of about 25% of the target daily dose of finerenone and/or a second dose of about 50% of the target daily dose of finerenone. In embodiments, after the patient has stopped receiving the CYP3A4 inhibitor, the methods comprise administering a first dose of about 25% of the target daily dose of finerenone for about 1-31 days and/or a second dose of about 50% of the target daily dose of finerenone for about 1-31 days.

In embodiments, after the patient has stopped receiving the strong CYP3A4 inhibitor, the methods comprise administering a first dose of finerenone comprising from about 10% to about 30% of the target daily dose of finerenone for about 2 to about 30 half-lives after stopping the strong CYP3A4 inhibitor, subsequently administering a second dose (e.g., for about 2 to about 30 half-lives after stopping the CYP3A4 inhibitor) of finerenone that is higher than the first dose of finerenone but lower than the target daily dose of finerenone, and subsequently administering the target daily dose of finerenone. In embodiments, after the patient has stopped receiving the strong CYP3A4 inhibitor, the methods comprise administering a first dose of finerenone comprising from about 10% to about 30% of the target daily dose of finerenone for about 3 to about 5 half-lives after stopping the strong CYP3A4 inhibitor, subsequently administering a second dose (e.g., about 3, 4, or about 5 half-lives after stopping the CYP3A4 inhibitor) of finerenone that is higher than the first dose of finerenone but lower than the target daily dose of finerenone, and subsequently administering the target daily dose of finerenone. In embodiments, the first dose of finerenone comprises from about 10% to about 30% (e.g., about 10%, about 15%, about 20%, about 25%, or about 30%) of the target daily dose of finerenone. In embodiments, the second dose of finerenone comprises from about 15% to about 85%, from about 15% to about 75%, from about 15% to about 65%, from about 15% to about 55%, or from about 15% to about 45% of the target daily dose of finerenone. In embodiments, the first and/or second dose is administered for about 2 to about 30 half-lives after stopping the strong CYP3A4 inhibitor. In embodiments, the first and/or second dose is administered for about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 half-lives after stopping the strong CYP3A4 inhibitor. In embodiments, after the patient has stopped receiving the strong CYP3A4 inhibitor, the methods comprise administering a first dose of about 25% of the target daily dose of finerenone and/or a second dose of about 50% of the target daily dose of finerenone. In embodiments, after the patient has stopped receiving the strong CYP3A4 inhibitor, the methods comprise administering a first dose of about 25% of the target daily dose of finerenone about 2-30 half-lives after stopping the strong CYP3A4 inhibitor and/or a second dose of about 50% of the target daily dose of finerenone for about about 2-30 half-lives after stopping the strong CYP3A4 inhibitor. In embodiments, after the patient has stopped receiving the strong CYP3A4 inhibitor, the methods comprise administering a first dose of about 25% of the target daily dose of finerenone about 2-30 half-lives after stopping the strong CYP3A4 inhibitor and/or a second dose of about 50% of the target daily dose of finerenone for about about 3-5 half-lives after stopping the strong CYP3A4 inhibitor.

In embodiments, while the patient is being coadministered a strong CYP3A4 inhibitor, the methods comprise administering the patient a first dose of finerenone comprising from about 10% to about 30% of the target daily dose of finerenone for about 1 to about 31 days, subsequently administering a second dose of finerenone that is higher than the first dose of finerenone but lower than the target daily dose of finerenone (e.g., for about 1 to about 31 days). In embodiments, the method further comprises subsequently administering the target daily dose of finerenone. In embodiments, the first dose of finerenone comprises from about 10% to about 30% (e.g., about 10%, about 15%, about 20%, about 25%, about 30%) of the target daily dose of finerenone. In embodiments, the second dose of finerenone comprises from about 15% to about 85%, from about 15% to about 75%, from about 15% to about 65%, from about 15% to about 55%, or from about 15% to about 45% of the target daily dose of finerenone. In embodiments, the first and/or second dose is administered for about 1 to about 31 days. In embodiments, the first and/or second dose is administered for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days. In embodiments, after a patient has initiated treatment with a CYP3A4 inhibitor, the methods comprise administering patient a first dose of about 25% of the target daily dose of finerenone and/or a second dose of about 50% of the target daily dose of finerenone. In embodiments, after a patient has initiated treatment with a CYP3A4 inhibitor, the methods comprise administering a first dose of about 25% of the target daily dose of finerenone for about 1-31 days and/or a second dose of about 50% of the target daily dose of finerenone for about 1-31 days.

### Experimental Part

### Abbreviations:

Abbreviations

| | |
|---|---|
| AE | Adverse event |
| ± or +/− | Plus/minus |
| ASCVD | Atherosclerotic cardiovascular disease |
| BP | Blood pressure |
| BUN | Blood urea nitrogen |
| CAD | Coronary artery disease |
| CHF | Chronic heart failure |
| CHF | Chronic heart failure |
| CKD | Chronic Kidney Disease |
| CKD-EPI | Chronic Kidney Disease Epidemiology Collaboration |
| CKM | Cardio-kidney-metabolic |
| CONUT | Controlling nutritional status |
| COPD | Chronic obstructive pulmonary disease |
| d | Day |
| D | Day |
| DBP | Diastolic blood pressure |
| dL | Decilitre |
| DM | Diabetes mellitus |
| ECG | Electrocardiogram |
| eGFR | Estimated glomerular filtration rate |
| EOS | End-of-study |
| EQ 5D 5L | EuroQol Group 5 dimension 5-level questionnaire |
| GNRI | Geriatric nutritional risk index |
| HF | Heart failure |
| HFmrEF | Heart failure with mid-range ejection fraction |
| HFpEF | Heart failure with preserved ejection fraction |
| HFrEF | Heart failure with reduced ejection fraction |
| HHF | Hospitalization for Heart failure |
| hs-TnT | High-sensitivity troponin-t |
| IxRS i | Interactive voice/web response system |
| K/K⁺ | Potassium |
| KCCQ | Kansas City Cardiomyopathy Questionnaire |
| KDIGO | The Kidney Disease: Improving Global Outcomes |
| kPa | Kilopascals |
| L | Liter |
| M | Month |
| MAGGIC | Meta-Analysis Global Group in Chronic (MAGGIC) |
| | Heart Failure Risk Score |
| med. | Medication |
| mEq | mmol |
| mg | Milligram |
| MI | Myocardial infarction |
| mL | Milliliter |
| mmHg | Millimeters of mercury |
| mmol | Millimole |
| mol | Mole |
| MR | Mineralocorticoid Receptor |
| MRA | Mineralocorticoid Receptor Antagonist |
| ng | Nanogram |
| NT-proBNP | N-terminal prohormone of brain natriuretic peptide |
| NYHA | New York Heart Association |
| O | On-site |
| OD | Once Daily |
| OSA | Obstructive sleep apnea |
| PAD | Peripheral artery diseas |
| PBPK | physiologically-based pharmacokinetics |
| PD | Premature discontinuation |
| pg | Picogram |
| PGIC | Patient Global Impression of Change |
| PGIS | PGI of Severity |
| PNI | Prognostic nutritional index |
| PT | Post-treatment |
| RAAS | Tenin-angiotensin-aldosterone system |
| SBP | Systolic blood pressure |
| Serum [K⁺] | Serum potassium level |
| SGLT2 | Sodium-glucose-transporter-2 |
| SoA | Schedule of activities |
| T2D | Type 2 DM |
| tel. | Telephone |
| TIA | Transient ischemic attack |
| WCHF | Worsening chronic heart failure |
| WHO | World Health Organization |
| yr | Year |

The following examples are offered to illustrate, but not to limit the claimed invention. The following examples further illustrate the invention but, of course, should not be construed as in any way limiting its scope.

The following examples are provided for purposes of illustration, not limitation. The following examples illustrate the various methods of administering or formulating compounds described herein. It is understood that one skilled in the art may be able to implement these procedures by similar methods or by combining other methods known to one skilled in the art. It is also understood that one skilled in the art would be able to make, in a similar manner as described below by using methods and tools well known in the art as needed.

### Example 1a—Measurements of Finerenone Polymorph

Lattice Constants of the Compound of Formula (I) in Crystalline Form of Polymorph I
Polymorph I
Crystal system orthorhombic
Space group P2 (1) 2 (1) 2 (1)
Molecules per unit
cell 4
Length of axis a [Ångstroms (Å)] 7.8610 (3)
Length of axis b [Å] 11.7797 (6)
Length of axis c [Å] 20.1792 (8)
α [°] 90
β [°] 90
γ [°] 90
Calculated density at 100 K [g·cm⁻³] 1.345

Measuring parameters of the x-ray diffractometry for the measurement of the compound of formula (I) in crystalline form of polymorph I

| | |
|---|---|
| Data set name | 2429-08a r2 |
| Scan axis | 2Theta-Omega |
| Start position [°2Th.] | 2.0000 |
| End position [°2Th.] | 37.9900 |
| Type of divergence screen | Fixed |
| Size of divergence screen [°] | 1.0000 |
| Measurement temperature [° C.] | 25 |
| Anode material | Cu |
| K-Alpha1 [Å] | 1.54060 |
| Generator setting | 35 mA, 45 kV |
| Diffractometer type | Transmission diffractometer |
| Goniometer radius [mm] | 240.00 |
| Focus-div. screen gap [mm] | 91.00 |
| Primary beam monochromator | Yes |
| Sample rotation | Yes |

[2 Theta (degrees (0)]
Polymorph I
8.5°
11.4°
11.9°
13.4°
14.1°
14.8°
15.0°
15.4°
16.0°
17.2°
18.5°
19.0°
19.8°
20.5°
20.8°
22.1°
22.7°
23.0°
23.1°
23.6°
23.9°
24.6°
24.9°
25.2°
25.6°
26.0°
26.5°
27.1°
27.3°
28.3°
28.5°
28.8°
29.6°
30.1°
30.6°
31.5°
31.9°
32.4°
32.9°
33.1°
33.4°
33.7°
34.5°
34.7°
35.0°
35.8°
36.2°
36.5°
37.2°
37.4°

Measuring Conditions for the IR and Raman Spectroscopy for the Measurement of the Compound of Formula (I) in Crystalline Form of Polymorph I:

| IR: | | |
|---|---|---|
| Instrument | Perkin Elmer Spectrum | |
| Number of scans | 32 | |
| Resolution | 4 | cm⁻¹ |
| Technique | Diamond ATR unit | |
| Raman: | | |
| Instrument | Bruker Raman RFS 100/S | |
| Number of scans | 64 | |
| Resolution | 2-4 | cm⁻¹ |
| Laser Power | 350 | mW |
| Laser wavelength | 1064 | nm |

Band maximum [cm⁻¹]

| 1R-ATR (cm⁻¹) | Raman (cm⁻¹) |
|---|---|
| Polymorph I | Polymorph I |
| 3475 | 3074 |
| 3416 | 2997 |
| 3366 | 2970 |
| 3074 | 2941 |
| 2992 | 2920 |
| 2952 | 2836 |
| 2835 | 2231 |
| 2230 | 1659 |
| 1681 | 1641 |
| 1658 | 1623 |
| 1606 | 1601 |
| 1572 | 1577 |
| 1485 | 1487 |
| 1464 | 1443 |
| 1454 | 1383 |
| 1431 | 1362 |
| 1420 | 1327 |
| 1407 | 1303 |
| 1381 | 1267 |
| 1355 | 1230 |
| 1341 | 1191 |
| 1325 | 1161 |
| 1303 | 1123 |
| 1285 | 1093 |
| 1267 | 1032 |
| 1255 | 991 |
| 1229 | 883 |
| 1222 | 827 |
| 1161 | 810 |
| 1136 | 759 |
| 1097 | 734 |
| 1031 | 708 |
| 991 | 671 |
| 976 | 613 |
| 967 | 528 |
| 924 | 505 |
| 909 | 471 |
| 875 | 442 |
| 847 | 346 |
| 827 | 320 |
| 810 | 297 |
| 776 | 186 |
| 758 | 155 |
| 746 | 114 |
| 733 | |
| 723 | |
| 706 | |
| 697 | |
| 670 | |

### Example 1b—Alternative Assays for Measuring Levels of NT-proBNP [Ng/L]

Levels of NT-proBNP, the N-terminal fragment of pro-natriuretic peptide, type B, can be determined using, for example, immunological assays such as the Roche Cobas Elecsys proBNPII or the Siemens Immulite. Both assays are immunological in vitro diagnostic tests for the quantitative determination of NT-proBNP in human plasma or serum and typically use monoclonal (or polyclonal) antibodies against NT-proBNP to capture and detect the analyte NT-proBNP. The absolute concentration of NT-proBNP in FINEARTS-HF samples was determined using either of the two assays. To account for assay-to-assay differences in the absolute NT-proBNP values between the Roche and Siemens protocols, samples analyzed on Siemens Immulite were adjusted (after analyzing matched samples on both instruments) to Roche Cobas NT-proBNP as primary read-out resulting in lower overall NT-proBNP values compared to the values obtained from the Siemens protocol. Throughout the specification and claims, NT-proBNP values determined by the above-described Siemens Immulite assay were identified by [symbol] following the NT-proBNP value. NT-proBNP values determined by the above described Roche Cobas assay were identified by [symbol] following the NT-proBNP value.

### Example 2—Study Design

### Study Objectives and Endpoints

Example 2 was a global, multicenter, placebo-controlled, event-driven randomized clinical trial investigating the long-term efficacy and safety of the non-steroidal MRA finerenone in symptomatic patients with HF with LVEF ≥40%. The trial was designed collaboratively by members of the steering committee and the sponsor. The trial has been registered on ClinicalTrials.gov (NCT04435626) and EudraCT (2020-000306-29).

### Study Design

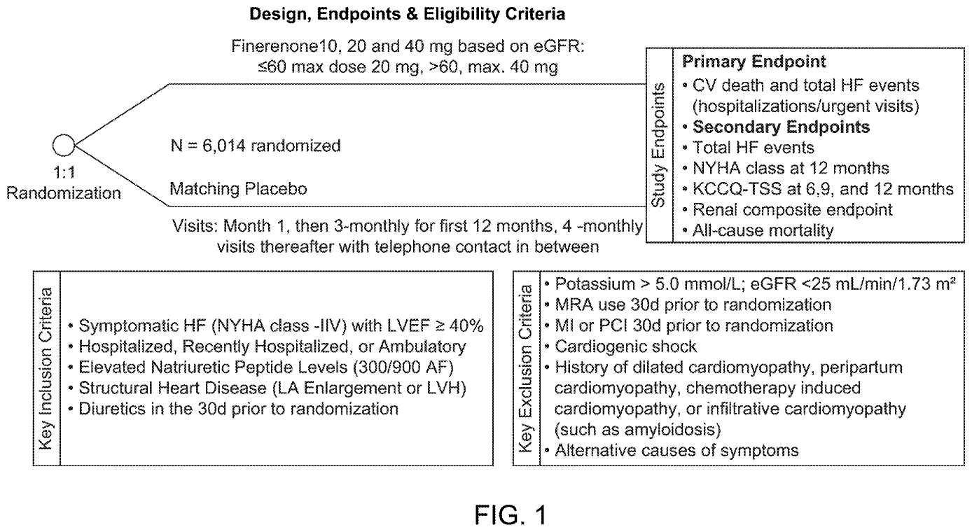

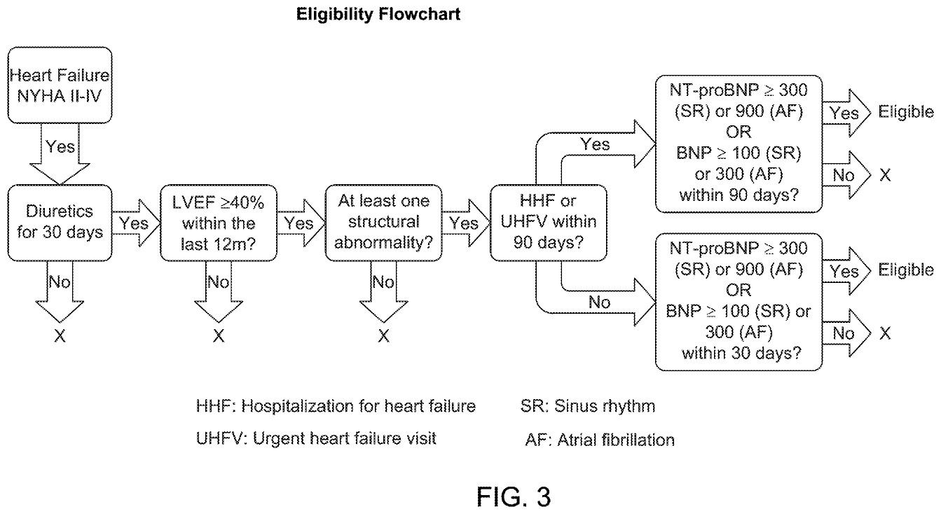

The Design, Endpoints and Eligibility criteria are shown in FIG. 1. In brief, FINEARTS-HF enrolled adults (≥40 years of age) with symptomatic HF (New York Heart Association class II-IV) with diuretic requirement at least 30 days prior to randomization. LVEF ≥40% and evidence of structural heart disease (left atrial enlargement or left ventricular hypertrophy) were required and measured locally by any modality within 12 months of screening. Left atrial diameter ≥3.8 cm, left atrial area ≥20 cm², or left atrial volume index >30 mL/m² qualified as left atrial enlargement. Left ventricular mass index ≥115 g/m² (if male) or ≥95 g/m² (if female) or septal or posterior wall thickness ≥1.1 cm qualified as left ventricular hypertrophy. Enrollment of patients with prior LVEF<40% with subsequent improvement to ≥40% (HFimpEF) was permitted provided that ongoing HF symptoms were present. The subgroup of patients with LVEF ≥60% was capped at a maximum of 20% of participants in the trial. All patients were required to have elevated natriuretic peptide levels, with eligibility thresholds adjusted based on the timing of recent HF events and the presence of atrial fibrillation. For patients in sinus rhythm, NT-proBNP ≥300 pg/mL (or BNP≥100 pg/mL) were required, measured within 30 days (in those without a recent worsening HF event) or within 90 days (in those with a recent worsening HF event). Qualifying levels of NT-proBNP or BNP were tripled if a patient was in atrial fibrillation/flutter at screening. A worsening HF event could include either a hospitalization for HF or an urgent visit for HF. An eligibility flowchart is presented in FIG. 3.

### Schedule of Activities (SoA)

The schedule of activities (SoA) is displayed for the study as a whole in Table 1 (‘Main SoA’) and for participants who prematurely discontinued the study, minimal assessments needed to be performed as outlined in Table 2 (‘Premature Discontinuation SoA’).

TABLE 1
(‘Main SoA’) - Study assessments and procedures by study period and visit are presented
Table 1 Main SoA

| Visit Number/Name | | | | | | | |
|---|---|---|---|---|---|---|---|
| | | | | | | | 7, 9, 11, 13, |
| | | | | | | | 15, 17, 19, 21 |
| Screening ^{a} | Base-line ^{a}1 | 2 | 3 | 4 | 5 | 6 | etc.^{b} |
| Day (D)/Month (M) | | | | | | | |
| | | | | | | | M 14 and |
| | | | | | | | every |
| | | | | | | | 4 months |
| | | | | | | | (i.e. M 18, |
| | | | | | | | M 22, M 26, |
| | | | | | | | M 30, M 34, |
| | | | | | | | M 38, M 42 |
| | D 1 | M 1 | M 3 | M 6 | M 9 | M 12 | etc.) |
| Visit window (days) | | | | | | | |
| | — | ±3 | ±3 | ±6 | ±6 | ±6 | ±7 |
| On-site (◯)/Tel. contact (□) | | | | | | | |
| ◯ | ◯ | ◯ | ◯ | ◯ | ◯ | ◯ | □ |

Initiation procedures

| | | | | | | | | |
|---|---|---|---|---|---|---|---|---|
| Informed | X | | | | | | | |
| consent | | | | | | | | |
| Demographic | X | | | | | | | |
| data | | | | | | | | |
| Substance | | X | | | | | | |
| use (alcohol & | | | | | | | | |
| tobacco) | | | | | | | | |
| Medical | X | | | | | | | |
| history | | | | | | | | |
| NYHA class | X | X | X | X | X | X | X | X |
| assessment | | | | | | | | |
| Prior and | X | X | X | X | X | X | X | X |
| concomitant | | | | | | | | |
| medication | | | | | | | | |
| In- and | X | X | | | | | | |
| exclusion | | | | | | | | |
| criteria | | | | | | | | |

Clinical procedures/assessments

| | | | | | | | | |
|---|---|---|---|---|---|---|---|---|
| Weight | X | X | X | X | X | X | X | |
| Height | X | | | | | | | |
| Waist | X | | | | | | | |
| and hip | | | | | | | | |
| circumference | | | | | | | | |
| Vital | X | X | X | X | X | X | X | |
| signs ^{g} | | | | | | | | |
| 12-lead | | X | | | | | | |
| ECG (local) | | | | | | | | |
| AE and | | X | X | X | X | X | X | X |
| endpoint | | | | | | | | |
| assessment | | | | | | | | |
| (renal | | | | | | | | |
| endpoints | | | | | | | | |
| require | | | | | | | | |
| additional | | | | | | | | |
| confirmed | | | | | | | | |
| creatinine | | | | | | | | |
| measurement) | | | | | | | | |

Study intervention

| | | | | | | |
|---|---|---|---|---|---|---|
| Randomization | X | | | | | |
| (IxRS) | | | | | | |
| Dispense study | X | X | X | X | X | X |
| intervention | | | | | | |
| Provide and | X | | | | | X |
| review the | | | | | | |
| study contact | | | | | | |
| card | | | | | | |
| Administration | X | | X | | | |
| of study | | | | | | |
| intervention | | | | | | |
| at study site | | | | | | |
| Administration | | X | | X | X | X |
| of study | | | | | | |
| intervention | | | | | | |
| before the | | | | | | |
| visit | | | | | | |
| Study | | X | X | X | X | X |
| intervention | | | | | | |
| accountability | | | | | | |

Local/central laboratory

| | | | | | | | |
|---|---|---|---|---|---|---|---|
| Local | X ^{j} | X ^{j, m} | X ^{k} | X ^{k} | X ^{k} | X ^{k} | X ^{k} |
| laboratory ^{h} | | | | | | | |
| (potassium | | | | | | | |
| and creatinine ⁱ) | | | | | | | |
| Pregnancy test | X ^{L} | X ^{L, m} | | | | | |
| Central | | X ^{m} | X | X | X ^{s} | X | X |
| laboratory | | | | | | | |
| including | | | | | | | |
| urinalysis | | | | | | | |
| Biomarkers | | X ^{m} | | X | | | X |
| NT-proBNP | | | | | | | |
| and hs-TnT | | | | | | | |
| Exploratory | | X ^{m} | | X | | | X |
| biomarkers | | | | | | | |
| Pharmaco- | | | | X ⁿ | | | X ^{o} |
| kinetics | | | | | | | |

Other study procedures

| | | | | |
|---|---|---|---|---|
| KCCQ ^{q} | X | X | X | X |
| EQ-5D-5L ^{q} | X | X | X | X |
| PGIC (applicable | | X | X | X |
| to selected | | | | |
| sites only) ^{q} | | | | |
| PGIS (applicable | X | X | X | X |
| to selected | | | | |
| sites only) ^{q} | | | | |

| | Visit Number/Name | | | | | |
|---|---|---|---|---|---|---|
| | | | Up-titration, | | | |
| | 8, 12, 16, 20 | | re-start and | PD | EOS | PT |
| | etc. | 10, 14, 18 etc. | safety check ^{c} | Visit ^{d} | Visit ^{e} | Visit ^{f} |
| | Day (D)/Month (M) | | | | | |
| | M 16 and | | | | | |
| | every | M 20 and | | | | |
| | 8 months | every | | | | |
| | (i.e. M 24, | 8 months | | | | |
| | M 32, M 40 | (i.e. M 28, | | | | |
| | etc.) | M 36 etc.) | | | | |
| | Visit window (days) | | | | | |
| | ±7 | ±7 | ±7 | | | ±5 |
| | On-site (◯)/Tel. contact (□) | | | | | |
| | ◯ | ◯ | ◯ | ◯ | ◯ | □ |
| Initiation procedures | | | | | | |
| Informed | | | | | | |
| consent | | | | | | |
| Demographic | | | | | | |
| data | | | | | | |
| Substance | | | | | | |
| use (alcohol & | | | | | | |
| tobacco) | | | | | | |
| Medical | | | | | | |
| history | | | | | | |
| NYHA class | X | X | | X | X | X |
| assessment | | | | | | |
| Prior and | X | X | X | X | X | X |
| concomitant | | | | | | |
| medication | | | | | | |
| In- and | | | | | | |
| exclusion | | | | | | |
| criteria | | | | | | |
| Clinical procedures/assessments | | | | | | |
| Weight | X | X | X | X | X | |
| Height | | | | | | |
| Waist | | | | | | |
| and hip | | | | | | |
| circumference | | | | | | |
| Vital | X | X | X | X | X | |
| signs ^{g} | | | | | | |
| 12-lead | | | | | | |
| ECG (local) | | | | | | |
| AE and | X | X | X | X | X | X |
| endpoint | | | | | | |
| assessment | | | | | | |
| (renal | | | | | | |
| endpoints | | | | | | |
| require | | | | | | |
| additional | | | | | | |
| confirmed | | | | | | |
| creatinine | | | | | | |
| measurement) | | | | | | |
| Study intervention | | | | | | |
| Randomization | | | | | | |
| (IxRS) | | | | | | |
| Dispense study | X | X | X ^{r} | | | |
| intervention | | | | | | |
| Provide and | | X | | | | |
| review the | | | | | | |
| study contact | | | | | | |
| card | | | | | | |
| Administration | | | | | | |
| of study | | | | | | |
| intervention | | | | | | |
| at study site | | | | | | |
| Administration | X | X | | | | |
| of study | | | | | | |
| intervention | | | | | | |
| before the | | | | | | |
| visit | | | | | | |
| Study | X | X | X | X | X | |
| intervention | | | | | | |
| accountability | | | | | | |
| Local/central laboratory | | | | | | |
| Local | X ^{k} | X ^{k} | X ^{k} | X ^{k} | X ^{k} | |
| laboratory ^{h} | | | | | | |
| (potassium | | | | | | |
| and creatinine ⁱ) | | | | | | |
| Pregnancy test | | | | | | |
| Central | X | X | | X | X | |
| laboratory | | | | | | |
| including | | | | | | |
| urinalysis | | | | | | |
| Biomarkers | | | | | | |
| NT-proBNP | | | | | | |
| and hs-TnT | | | | | | |
| Exploratory | | | | | X | |
| biomarkers | | | | | | |
| Pharmaco- | | X ^{o} | | | | |
| kinetics | | | | | | |
| Other study procedures | | | | | | |
| KCCQ ^{q} | X | | | X | X | |
| EQ-5D-5L ^{q} | X | | | X | X | |
| PGIC (applicable | | | | | | |
| to selected | | | | | | |
| sites only) ^{q} | | | | | | |
| PGIS (applicable | | | | | | |
| to selected | | | | | | |
| sites only) ^{q} | | | | | | |

TABLE 2
Premature Discontinuation SoA
Table 2 Premature Discontinuation SoA

| | Visit Number/Name | | | | | | | | | |
|---|---|---|---|---|---|---|---|---|---|---|
| | | | | | | | 7, 9, 11, | 8, 12, 16, | 10, 14, | |
| | | | | | | | 13, | 20 | 18 | EOS |
| | | 2 | 3 | 4 | 5 | 6 | etc.b | etc. | etc. | Visit e |
| | Day (D)/Month (M) | | | | | | | | | |
| | | | | | | | | M 16 every | | |
| | | | | | | | M 14 every | 8 months | M 20 every | |
| | | | | | | | 4 months | (i.e. M | 8 months | |
| | | | | | | | (i.e. M 18, | 24, M 32, | (i.e. M 28, | |
| | | M 1 | M 3 | M 6 | M 9 | M 12 | M 22 etc.) | M 40 etc.) | M 36 etc.) | |
| | Visit window (days) | | | | | | | | | |
| | | ±3 | ±3 | ±6 | +6 | ±6 | ±7 | ±7 | ±7 | |
| | On-site (◯)/Tel. contact (□) | | | | | | | | | |
| | | □ | ◯ | ◯ | □ | ◯ | □ | ◯ | ◯ | ◯ |
| Central | Premature | | X | X | | X | | X | X | X |
| laboratory | discontinuation ^{p} | | | | | | | | | |
| (eGFR) | | | | | | | | | | |
| Biomarkers | | | X | | | X | | | | |
| NT-proBNP | | | | | | | | | | |
| and hs-TnT | | | | | | | | | | |
| AE and | | X | X | X | X | X | X | X | X | X |
| endpoint | | | | | | | | | | |
| assessment | | | | | | | | | | |
| (renal | | | | | | | | | | |
| endpoints | | | | | | | | | | |
| require | | | | | | | | | | |
| additional | | | | | | | | | | |
| confirmed | | | | | | | | | | |
| creatinine | | | | | | | | | | |
| measurement) | | | | | | | | | | |
| Concomitant | | X | X | X | X | X | X | X | X | X |
| medication | | | | | | | | | | |
| KCCQ ^{q} | | | | X | | X | | X | | X |
| EQ-5D-5L ^{q} | | | | X | | X | | X | | X |

### Please Note:

1 month corresponds to 30 days
Study visits occurred as close as possible to the specified time points in the protocol, but time windows were permitted as specified in the SoA
At any scheduled or unscheduled visit, the dose of study intervention was checked whether to be increased to the next possible higher dose, based on serum/plasma potassium level analyzed in the local laboratory and provided the participant was already on a stable dose for 4 weeks ±7 days.

Abbreviations: AE=adverse event; CKD-EPI=Chronic Kidney Disease Epidemiology Collaboration; D=Day; eGFR=estimated glomerular filtration rate; ECG=electrocardiogram; EOS=end-of-study; EQ-5D-5L=EuroQoL Group 5-dimension 5-level questionnaire; hs-TnT=high-sensitivity troponin-t; IxRS=interactive voice/web response med. system; =medication; KCCQ=Kansas City Cardiomyopathy Questionnaire; M=Month; NT-proBNP=n-terminal prohormone B-type natriuretic peptide; NYHA=New York Heart Association; O=on-site; PGIC=Patient Global Impression of Change; PGIS=PGI of Severity; PD=premature discontinuation; PT=post-treatment; SoA=schedule of activities; Tel.=telephone
a Randomization occurred within 2 weeks of the Screening Visit. If the Screening Visit and Visit 1 (Day 1, Baseline) were performed on the same day, procedures listed for both visits were performed only once.
b Study visits were conducted as a clinic visit or a telephone contact visit. These visits were alternate at 4-monthly intervals from Month 12 onwards with participant contact being made every 2 months.
c This visit was performed for safety check after any up-titration (4 weeks±7 days) and after restart of study intervention following an interruption for >7 consecutive days. A full unscheduled safety visit was performed within an adequate timeframe proposed by the investigator after down-titration for reasons other than hyperkalemia, otherwise only potassium has been rechecked.
d The PD Visit took place as soon as possible but within 7 days after the last intake of study intervention. If the PD Visit could not be performed within the timeframe specified, no PD Visit was required. All randomized participants had to follow until the study ends, even if they did not take study intervention or permanently discontinued study intervention.
e After the study site was notified of end of study decision, an EOS Visit has been scheduled as soon as possible (but within 4 weeks at the latest).
f For all participants who were still on treatment with study drug at the EOS Visit, the post-treatment (PT) telephone call (D) was intended to be performed 30 days±5 days after the last intake of study drug.
g For vital sign collection
h If BNP, NT-proBNP values (related to inclusion criteria) were not available in medical records, values assessed by local laboratory were used.
i Creatinine was used to calculate eGFR using CKD-EPI
j If local laboratory data for potassium and eGFR were available within the last 24 hours, these could be used instead. This also applied when screening and baseline visits were not combined.
k Study participants may had their local laboratory assessments taken up to 3 days prior to the study visit.
L Female participants of childbearing potential had to have a negative serum or urine pregnancy test at screening and baseline. Further serum or urine pregnancy tests were performed in participants of childbearing potential as required by national/institutional regulations (e.g., at every visit). At any time during study participation, additional pregnancy testing could be performed upon suspicion of pregnancy.
m All procedures at Visit 1 were to be performed prior to randomization.
n One trough sample was to be collected at steady state before study intervention intake at Visit 3 (if not possible, e.g., because study intervention was taken at home before the visit, the trough sample collection could be postponed to Visit 4 or 5); study intervention was to be administered at the study site at this visit.
Sample should be taken during the visit 1.5-10 hours after study intervention intake at home.
p The procedures/assessments should be performed at the PD Visit are listed in the main SoA. After completing the PD Visit, all subsequent visits were to be performed according to the Premature Discontinuation SoA (Table 2-Premature Discontinuation SoA Table 2). Any visits performed prior to the PD Visit did not have to be repeated (e.g., if PD is at Visit 5, there was no need to repeat previous visits).
q Questionnaires were to be completed by the participants before conducting any study procedure.
r Only if applicable.
s At Visit 4 an additional NT-proBNP measurement from serum could be performed.

### Eligibilities

Eligibility Criteria. Final inclusion and exclusion criteria are summarized below. Table 3, Table 4 and Table 5 depict summaries of some selected aspects of the study:

TABLE 3
Key inclusion and key exclusion criteria

| Key Inclusion Criteria | Key Exclusion Criteria |
|---|---|
| Symptomatic HF (NYHA class II-V) | Potassium >5.0 mmol/L; eGFR <25 |
| with LVEF ≥40% | mL/min/1.73 m² |
| Hospitalized, Recently | MRA use 30 d prior to randomization |
| Hospitalized, or Ambulatory | MI or PCI 30 d prior to randomization |
| Elevated Natriuretic Peptide | Cardiogenic shock |
| Levels (300/900 AF) | History of dilated cardiomyopathy, |
| Structural Heart Disease (LA | peripartum cardiomyopathy, chemotherapy |
| Enlargement or LVH) | induced cardiomyopathy, or infiltrative |
| Diuretics in the 30 d prior to | cardiomyopathy (such as amyloidosis) |
| randomization | Alternative causes of symptoms |

TABLE 4
Study Endpoints

| Primary Endpoint | Secondary Endpoints |
|---|---|
| CV death and total HF events | Total HF events |
| (hospitalizations/urgent visits) | NYHA class at 12 months |
| | KCCQ-TSS at 6, 9, and 12 months |
| | Renal composite endpoint |
| | All-cause mortality |

TABLE 5
Main inclusion and main exclusion criteria - Example
2 included patients with LVEF ≥40% and eGFR ≥25 ml/min/1.73
m², with or without diabetes mellitus DM.

| Main inclusion criteria | Main exclusion criteria |
|---|---|
| Aged ≥40 years | eGFR <25 mL/min/1.73 m² |
| HF diagnosis with NYHA class II-IV | Serum [K⁺] >5.0 mmol/L |
| LVEF ≥40% | MI or any event that could have |
| eGFR ≥25 mL/min/1.73 m² | reduced the EF within 90 days |
| Serum [K⁺] ≤5.0 mmol/L | prior to randomisation |
| In patients with | Systolic blood pressure |
| sinus rhythm: NT-proBNP ≥300 pg/mL | (SBP) ≥160 mmHg |
| or BNP ≥100 pg/mL | |
| In patients with AF: NT-proBNP ≥900 | |
| pg/ml or BNP ≥300 pg/mL | |

### Detailed Example 2

### (1) Study Population

Patients with a diagnosis of HF, NYHA class II-IV, and documented LVEF of ≥40%. In the following, an overview of the inclusion and exclusion criteria as well as the patient population is summarized.

a. Inclusion Criteria

Participants were eligible to be included in the study only if all of the following criteria apply: Main inclusion criteria were:
Aged ≥40 years
HF diagnosis with NYHA class II-IV
LVEF ≥40%
eGFR ≥25 mL/min/1.73 m²
Serum [K⁺]≤5.0 mmol/L
In patients with sinus rhythm: [K⁺] NT-proBNP ≥300 μg/mL, or BNP ≥100 pg/mL.

### Age

Participant must be aged 40 years and older, at the time of signing the informed consent.

### Type of Participant and Disease Characteristics:

Diagnosis of heart failure with NYHA class II-IV, ambulatory or hospitalized primarily for heart failure (if a hospitalized patient could not be randomized as an in-patient, randomization as soon as possible after discharge was encouraged).
On diuretic treatment for at least 30 days prior to randomization.
Documented LVEF of ≥40% measured by any modality within the last 12 months, at the latest at screening; if several values were available, the most recent one should be reported. If LVEF was not measured in the past 12 months, a new measurement could be done at screening.
Structural heart abnormalities based on any local imaging measurement within the last 12 months, latest at screening, defined by at least 1 of the following findings:
LAD ≥3.8 cm, LAA ≥20 cm², LAVI >30 mL/m², LVMI ≥115 g/m² (3)/95 g/m² (9), septal thickness or posterior wall thickness ≥1.1 cm.
NT-proBNP ≥300 pg/mL (BNP ≥100 pg/mL) in sinus rhythm and patient did not have an ongoing diagnosis of paroxysmal atrial fibrillation or NT-proBNP ≥900 pg/mL (BNP ≥300 pg/mL) in atrial fibrillation (or if atrial fibrillation status was unknown or if patient had an ongoing diagnosis of paroxysmal atrial fibrillation) for participants obtained at the following time:
Within 90 days prior to randomization if patient had been hospitalized for HF requiring initiation or change in HF therapy or if patient had an urgent visit for HF requiring intravenous (IV) diuretic therapy, both within 90 days prior to randomization.

OR
Within 30 days prior to randomization if patient had not been hospitalized for HF nor had an urgent HF visit within the past 90 days.

### Sex

### Male or Female.

Women of childbearing potential could only be included in the study if a pregnancy test was negative at screening and baseline and if they agreed to use adequate contraception which was consistent with local regulations regarding the methods for contraception for those participating in clinical trials.

### Informed Consent

Capable of giving signed informed consent which included compliance with the requirements and restrictions listed in the informed consent form (ICF) and in this protocol.

b. Exclusion Criteria

Main exclusion criteria were:
eGFR <25 mL/min/1.73 m²
Serum [K⁺]>5.0 mmol/L
MI or any event that could have reduced the EF within 90 days prior to randomization
SBP ≥160 mmHg

Participants were excluded from the study if any of the following criteria applied:

### Medical Conditions

eGFR <25 mL/min/1.73 m² at either screening or randomization visit. NOTE: one reassessment of eGFR was allowed at the screening and randomization visit, respectively.
Serum/plasma potassium >5.0 mmol/L at either screening or randomization visit. NOTE: one reassessment of potassium was allowed at the screening and randomization visit, respectively.
Acute inflammatory heart disease, e.g., acute myocarditis, within 90 days prior to randomization.
Myocardial infarction or any event which could have reduced the ejection fraction within 90 days prior to randomization.
Coronary artery bypass graft surgery in the 90 days prior to randomization.
Percutaneous coronary intervention in the 30 days prior to randomization.
Stroke or transient ischemic cerebral attack within 90 days prior to randomization.
Probable alternative cause of participants' HF symptoms that in the opinion of the investigator was primarily responsible for patient's dyspnea such as significant pulmonary disease, anemia or obesity. Specifically, patients with the below were excluded:
Severe pulmonary disease requiring home oxygen, or chronic oral steroid therapy;
History of primary pulmonary arterial hypertension;
Hemoglobin <10 g/dL;
Valvular heart disease considered by the investigator to be clinically significant;
Body mass index (BMI)>50 kg/m² at screening;
Systolic blood pressure (SBP) ≥160 mmHg if not on treatment with ≥3 blood pressure lowering medications or ≥180 mmHg irrespective of treatments on 2 consecutive measurements at least 2-minute apart, at screening or at randomization;
Life-threatening or uncontrolled arrhythmias at screening and/or randomization including, but not limited to, sustained ventricular tachycardia and atrial fibrillation, or atrial flutter with resting ventricular rate >110 bpm;
Symptomatic hypotension with mean systolic blood pressure <90 mmHg at screening or at randomization;
Any primary cause of HF scheduled for surgery, e.g., valve disease such as severe aortic stenosis or severe mitral regurgitation by the time of screening or randomization;
History of peripartum cardiomyopathy, chemotherapy induced cardiomyopathy, viral myocarditis, right heart failure in absence of left-sided structural disease, pericardial constriction, genetic hypertrophic cardiomyopathy, or infiltrative cardiomyopathy including amyloidosis;
Presence of left ventricular assist device by the time of screening or randomization;
History of hyperkalemia or acute renal failure during MRA treatment for >7 consecutive days, leading to permanent discontinuation of the MRA treatment;
Pregnant or nursing (lactating) women, where pregnancy is defined as the state of a female after conception and until the termination of gestation, confirmed by a positive human chorionic gonadotrophin urine or serum test;
Known hypersensitivity to the study intervention (active substance or excipients);
Hepatic insufficiency classified as Child-Pugh C at screening or randomization; or
Addison's disease.

### Prior/Concomitant Therapy

Requirement of any IV vasodilating drug (e.g., nitrates, nitroprusside), any IV natriuretic peptide (e.g., nesiritide, carperitide), any IV positive inotropic agents, or mechanical support (e.g., intra-aortic balloon pump, endotracheal intubation, mechanical ventilation, or any ventricular assist device) within 24 hours prior to randomization.
Participants who required treatment with more than one ACEI, ARB or angiotensin-receptor neprilysin inhibitor (ARNI), or two simultaneously at randomization.
Continuous (at least 90 days) treatment with an MRA (e.g., spironolactone, eplerenone, canrenone, esaxerenone) within 12 months prior to screening. Last intake at least 30 days before randomization. Treatment with MRA should not be interrupted with the purpose of enrollment into the study.
Concomitant treatment with any renin inhibitor or potassium-sparing diuretic that could not be stopped prior to randomization and for the duration of the treatment period.
Concomitant systemic therapy with potent cytochrome P450 isoenzyme 3A4 (CYP3A4) inhibitors (e.g., itraconazole, ritonavir, indinavir, cobicistat, clarithromycin) or moderate or potent CYP3A4 inducers, that could not be discontinued 7 days prior to randomization and for the duration of the treatment period.
Concomitant systemic therapy with potent cytochrome P450 isoenzyme 3A4 (CYP3A4) inhibitors was equivalent to strong cytochrome 3A4 (CYP3A4) inhibitors.

### Other Exclusions

Any other condition or therapy, which would have made the participant unsuitable for this study and would not have been allowed participation for the full planned study period (e.g., active malignancy or other condition limiting life expectancy to less than 12 months).
Previous assignment to treatment during this study.
Participation in another interventional clinical study (e.g., Phase 1 to 3 clinical studies) or treatment with another investigational medicinal product within 30 days prior to randomization.
Close affiliation with the investigational site; e.g. a close relative of the investigator, dependent person (e.g., employee or student of the investigational site).
Known current alcohol and/or illicit drug abuse that could have interfered with the participant's safety and/or compliance at the discretion of the investigator.
Participant was in custody by order of an authority or a court of law.

In brief, Example 2 enrolled adults (≥40 years) with symptomatic HF (New York Heart Association class II-IV) with diuretic requirement at least 30 days prior to randomization. LVEF ≥40% and evidence of structural heart disease (left atrial enlargement or left ventricular hypertrophy) were required and could be measured locally by any modality within 12 months of screening. Previous LVEF that was <40% that had since improved to ≥40% was permitted. The subgroup of LVEF ≥60% was capped at a maximum of 20% of participants in the trial. All patients were required to have elevated natriuretic peptide levels, with differential recency of laboratory measurement and thresholds based on rhythm status and recent worsening HF event. In patients in sinus rhythm, NT-proBNP ≥300 pg/mL (or BNP≥100 pg/mL) were required measured within 30 days (in those without a recent worsening HF event) or within 90 days (in those with a recent worsening HF event). Qualifying levels of NT-proBNP or BNP were tripled if a patient was in atrial fibrillation/flutter at screening.

Patients could be enrolled irrespective of clinical care setting (whether hospitalized, recently hospitalized, or ambulatory). The proportion of patients without a recent worsening HF event within 3 months of randomization was capped globally at approximately 50% of the original planned sample size.

Estimated glomerular filtration rate <25 mL/min/1.73 m² or serum/plasma potassium >5.0 mmol/L at screening or randomization were key exclusion criteria. Recent treatment with an MRA within 12 months of screening was not permitted, and discontinuation of an MRA for the purposes of study enrollment was discouraged.

All participants signed informed consents for participation and the study protocol was approved by the ethics committees and institutional review boards for each participating site. The trial started on Sep. 14, 2020 and had validly randomized over 6,000 participants across 634 sites in 37 countries. The study was conducted in accordance with Good Clinical Practice and the Declaration of Helsinki.

### Study Design

Following a screening period of up to 2 weeks, eligible participants were randomized in a 1:1 ratio to either finerenone or matching placebo. The randomization was stratified by country/region and LVEF (<60%, ≥60%). Starting dose was selected based on baseline eGFR. Participants with an eGFR ≤60 mL/min/1.73 m² started 10 mg once daily with a maximum maintenance dose of 20 mg once daily, whereas participants with an eGFR >60 mL/min/1.73 m² started 20 mg once daily with a maximum maintenance dose of 40 mg once daily (Table 3).

In this ongoing study, patients were followed with 2 scheduled visits within the first 3 months of randomization, and then every 3 months until 1 year. After 1 year, telephone follow-up 1 alternated with on-site visits every 2 months until the end of the trial.

After the completion of the randomized phase, any participant were still taking the study drug took part in a post-treatment follow-up period for 30 days.

### Dose Titration and Monitoring of Safety and Tolerability

The study aimed to encourage reaching the maximum maintenance dose and sustaining it as long as safe and tolerated. Blood measurements of potassium and creatinine were performed serially at study visits to capture safety events. Up-titration was expected to occur any time after 4 weeks based on serum/plasma potassium level and if eGFR decrease has not been exceeded ≥30%. Down-titration could occurred at any time if potassium ≥5.5 mEq/L and temporarily interrupted if already at the lowest dose.

### Study Objectives

The primary objective was to demonstrate the superiority of finerenone to placebo in reducing the rate of the composite of cardiovascular death and total (first and recurrent) HF events (HF hospitalization or urgent HF visit). The secondary objectives were to determine the superiority of finerenone to placebo for each secondary endpoint: total HF events; improvement in NYHA class from baseline to Month 12; change from baseline to Month 6, 9 and 12 in total symptom score of the Kansas City Cardiomyopathy Questionnaire (KCCQ); time to first occurrence of composite renal endpoint (defined as sustained decrease in eGFR ≥50% relative to baseline over at least 4 weeks, or sustained eGFR decline <15 mL/min/1.73 m², or initiation of dialysis or renal transplantation); time to all-cause mortality; and the safety and tolerability of finerenone.

### Statistical Considerations

The primary analysis had evaluated the total occurrences of the composite of cardiovascular death, hospitalization for HF, and urgent HF events. It was performed in the full analysis set (FAS) of all patients that had been randomized and had no major good clinical practice violations. The primary analysis of the primary composite endpoint was based on a Lin, Wei, Yang and Ying (LWYY) model (Lin et al., J.R.Statist.Soc. B (2000) 62, Part 4, pp. 711±730. https://doi.org/10.1111/1467-9868.00259). This is equivalent to the stratified Andersen-Gill model with use of robust standard errors (sandwich estimator) to account for correlations of event times within a participant. The model included treatment group as a fixed effect and including geographic region and baseline LVEF (<60%, ≥60%) as stratification factors. A small adjustment has been made to the nominal significance level at the final analysis to take into account the interim analysis. A point estimate of the rate ratio together with a 95% confidence interval (CI) was presented. Events and follow-up time after discontinuation of treatment was included in the analysis. Non-CV death was assumed to be a censoring event, since the treatment was not assumed to have an effect on these events and interest lied in the treatment effect on composite events while patients were still alive.

If a participant experiences an HF event and subsequently died for a cardiovascular reason, this was considered as two separate events for the primary analysis unless the subject died on the same calendar day as the HF event (both events would still be considered for the analyses of the separate components). If a subject was hospitalized for HF shortly after an urgent HF visit, this was considered as two separate events for the primary analysis unless they occur on the same calendar day.

The primary analysis method had been investigated with extensive simulation studies and it had been confirmed that it kept the alpha level and had good operating characteristics across a range of plausible scenarios, see also Fritsch et al. Statistics in Biopharmaceutical Research, 15 (2), 268-279. https://doi.org/10.1080/19466315.2021.1945488).

The trial was designed to show an effect on the primary endpoint with a power of 90% at a two-sided alpha of 5%. The sample size determination was based on a simulation study assuming a joint frailty model in order to account for the correlation between HF events and cardiovascular death, and to model participant heterogeneity with respect to baseline intensities/hazards (Rogers, et al. 2016, Statist. Med. 2016, 35 2195-2205. https://doi.org/10.1002/sim.6853). The placebo rate parameter of the Poisson process and the hazard rate of the exponential distribution were first chosen as 0.014 HHFs/month per participant and 0.004 CV deaths/month per participant, respectively. These values lead to an observed annualized placebo rate of first composite events of 9.0 (events/100 participant-years) and an observed annualized placebo rate of CV death of 3.5. These observed rates were similar to rates observed in the literature, i.e. an annualized rate of first composite event of 9.1 was observed in the CHARM-Preserved trial, 8.9 was observed in PARAGON-HF, and 8.5 in the BNP stratum of the TOPCAT trial. Regarding CV death, an annualized placebo rate of 3.9 per 100 participant-years was observed in CHARM-Preserved, 3.1 was observed in PARAGON-HF, and 3.9 in the TOPCAT BNP stratum. Since Example 2 was planned to recruit more participants with a very recent hospitalization than in previous trials, which would have been at a higher risk of events, the rate parameters were subsequently increased by 25% for CV death leading to a rate of 0.005125 CV deaths/month per participant. For HF events, the rate was increased by 30% to 0.0182 HF events/month per participant to also account for the inclusion of urgent HF visits. As treatment effects, a hazard ratio for CV death of 0.8 and a rate ratio for heart failure events of 0.75 were assumed.

It was originally anticipated that 5,500 participants would be randomized. A total of approximately 2,375 total (first and recurrent) primary composite events were targeted. Due to blinded event rates being lower than those assumed in the sample size calculation, the planned number of randomized subjects was increased to approximately 6,000.

The secondary hypotheses was formally tested, and statistical inferences was made only if the primary hypothesis was rejected. Secondary endpoints was tested sequentially: total HF events, NYHA class and KCCQ (tested using the Bonferroni-Holm procedure), composite renal endpoint. If the primary hypothesis was rejected, then all-cause mortality wa tested at a full two-sided significance level of 5% outside this hierarchy.

Total HF events was analyzed using an LWYY model in a similar fashion to the primary endpoint. The percentage of participants with improvement in NYHA class from Baseline to Month 12, was analyzed with a logistic regression model including factors for treatment group and stratification levels. Patients that died or are lost to follow-up at month 12 was considered to had not improved. The absolute change from baseline including measurements up to Month 12 of the TSS of the KCCQ, was analyzed by a repeated measures mixed model including the factors treatment group, baseline, visit, baseline-by-visit interaction, and factors for the stratification levels. The comparison assumed a common treatment effect across Month 6, 9 and 12. All observed values was included into the analysis regardless of treatment discontinuation. The composite renal endpoint and all-cause mortality was analyzed using a stratified log-rank test for testing and a stratified Cox proportional hazards model for obtaining a point estimate with 95% confidence interval. The Cox proportional hazards model was stratified according to the stratification factors and included treatment group as a fixed effect.

### Interim Analyses

One non-binding interim analysis for futility was planned when approximately 30% of the required total number of primary endpoint events were observed. If the observed rate ratio on the primary endpoint was above 0.95, the trial was planned to be stopped for futility. In addition, one formal interim analysis for efficacy was planned when approximately ⅔ of the required total number of primary endpoint events were observed. If the interim analysis showed clear and consistent benefit in the finerenone treatment group (primary efficacy endpoint with two-sided p-value<0.0027 and cardiovascular death component with nominal two-sided p-value<0.05), the Data Monitoring Committee may have recommended early study termination. A group sequential design with a single interim analysis when ⅔ of the information was available with a stopping rule of two-sided p<0.00270 would have require a small adjustment to the alpha level at the final analysis to maintain the overall significance level at 0.05. For an information fraction of ⅔, the adjusted alpha level of 0.04967 applied. In the event that the study was not stopped early for success a p-value of p<0.04967 was therefore required at the final analysis to achieve formal statistical significance.

### Discussion

Example 2 was a broadly inclusive trial of patients with HF with mildly reduced or preserved ejection fraction that was positioned to shed light on several key areas of uncertainty. While MR blockade in this target population had a strong theoretical basis, the clinical evidence had not been definitive. In Example 2, finerenone was tested against a placebo comparator as steroidal MRAs currently lack any specific regulatory labeling or class I recommendation for use in patients with HF with mildly reduced or preserved ejection fraction, and thus were not considered a part of global standard of care. The design of the trial had been informed by learnings from previous large-scale outcomes trials of various pharmacotherapies. Example 2 was large (with over 6,000 patients enrolled) and targeted a cohort at high-risk for disease progression, and thus was well-powered to definitively establish whether MRAs have a role in this patient population. The primary endpoint of the trial was intended to reflect a broader look at “worsening HF” by including cardiovascular death and total (not just first) HF events. Finally, dosing of finerenone had been optimized and guided by a phase 2 program, in particular the ARTS-HF trial (Filippatos et al., European Heart Journal, Volume 37, Issue 27, 14 Jul. 2016, Pages 2105-2114. 2016, https://doi.org/10.1093/eurheartj/ehw132), in which treatment effects on natriuretic peptide levels appeared to be dose dependent. Example 2 was intended to uniquely test a 40 mg target maintenance dose in people with an eGFR >60 mL/min/1.73 m², which was not available in the treatment of CKD at the time of starting this study.

The trial was also enriched to examine safety and efficacy of finerenone in a number of key subgroups. The trial was intended to have the highest percentage of hospitalized or recently hospitalized patients of any contemporary HFmrEF/HFpEF outcomes trial. As global guidelines now support in-hospital and early post-discharge optimization of guideline directed medical therapies, Example 2 was intended to directly inform this implementation strategy in the care of high-risk patients with HFmrEF/HFpEF. As most previous trials of steroidal MRAs had enrolled patients in stable ambulatory care, the safety of MRA initiation in this high-risk window of a worsening HF episode was less certain. A previous trial examined the early safety of high-dose spironolactone (administered at 100 mg daily) in 360 patients with acute HF, however MRA therapy was not continued post-discharge in this trial. Furthermore, Example 2 was adequately represented across the range of ejection fraction such that the therapeutic effects of MRA in patients with LVEF ≥60% will be clarified.

The study adds data to a better understanding of the role of finerenone across the spectrum of cardio-renal-metabolic health. To date, large-scale outcomes trials of finerenone in CKD had been restricted to those with concomitant diabetes. Example 2 was intended to be the first outcomes trial to test the efficacy and safety of finerenone in patients without diabetes and across a broad range of renal function (down to 25 mL/min/1.73 m²) and should complement data from the FIND-CKD trial (ClinicalTrials.gov NCT05047263), a dedicated trial of finerenone in non-diabetic CKD on change in total eGFR slope. Steroidal MRA use in indicated populations of HF is particularly low in patients with comorbid CKD, so Example 2 affirming safety of finerenone in this range of kidney function would add confidence to its use at this high-risk intersection. Indeed, a participant-level pooled analysis of Example 2, FIDELIO-DKD, and FIGARO-DKD was prespecified and would help map finerenone's therapeutic effects across cardio-renal-metabolic disease states.

The Example 2 trial was intended to determine the efficacy and safety of the non-steroidal MRA finerenone in a broadly inclusive population of hospitalized and ambulatory patients with mildly reduced or preserved ejection fraction. Example 2 has the potential to inform the safe application and implementation of finerenone in this target population.

### (2) Further Statistical Analyses

In addition to the main analysis of the primary and secondary endpoints described above sensitivity and supportive analyses was performed. Unless otherwise specified, all analyses was performed in the full analysis set.

Plots and summaries of the mean cumulative function for the primary endpoint (Nelsen-Aalen estimate) was presented by treatment group. The cumulative incidence functions for time to CV death and time to non-CV death was also calculated using Aalen-Johansen estimates.

As supportive analysis, a time to first composite of HF event or CV death analysis has been performed using a stratified Cox proportional hazard regression analysis. A plot of Aalen-Johansen estimates of the cumulative incidence function was provided.

As a sensitivity analysis, the number of primary composite events was also analyzed using a negative binomial regression model including stratification factors and treatment group as covariates and log follow-up time as an offset parameter. A further sensitivity analysis has included baseline LVEF as continuous covariate into the primary analysis model instead of the stratification factor LVEF (<60%, ≥60%).

As further sensitivity analysis, plots and summaries of the mean cumulative function for the primary endpoint has been derived based on a competing-risk approach (Ghosh and Lin, 2000, BIOMETRICS 56, June (2000), 554-562. https://doi.org/10.1111/j.0006-341X.2000.00554.x)

A separate estimate of the treatment effect for CV death as one of the components of the primary endpoint was intended to be obtained. The main cause-specific treatment effect estimate for CV death was intended to be derived from a stratified Cox proportional hazards model for time to CV death and the main p-value from a stratified log-rank test.

A supportive analysis of the primary endpoint has excluded urgent HF visits and considered only CV deaths and HHFs as events. Also, an additional analysis of the primary endpoint restricted CV deaths to HF-related events and thus considered HF events and CV deaths due to HF. These analyses have been performed for total (first and recurrent) events and for first events only.

A total-time approach considering times from randomization to the onset of first, second, third composite event using a Wei, Lin, and Weissfeld Journal of the American Statistical Association, 84 (408), 1065-1073. https://doi.org/10.1080/01621459.1989.10478873) model has been applied. This model enabled analysis of the cumulative effect on the primary endpoint from randomization (i.e., the effect on second event includes the effect on the first, and the effect on third event includes the effects on the first and second). In addition, a conditional gap-time model according to Prentice, Williams and Peterson Biometrika, Volume 68, Issue 2, August 1981, Pages 373-379. https://doi.org/10.1093/biomet/68.2.373) was applied to obtain HR estimates with 95% CIs for the time from first to second and from second to third event (note that this gives a non-randomized comparison). Both models employ robust standard errors and include the stratification factors and treatment group as fixed effects.

An “on-treatment” analysis has been performed, including only events occurring up to 30 days after treatment discontinuation and within 5 months after the last visit with complete information on all components of the composite primary endpoint. A 5-month time window was used as visits are 4-monthly and in order to allow for late attendance by an additional 1 month. The on-treatment analysis was done in the safety analysis set including participants in the full analysis set that have been treated.

The primary analysis for the primary endpoint was also repeated for the “Total HF events and all-cause mortality” endpoint.

The primary analysis was repeated where patients were included with only up to a maximum of 4 composite events, to examine the impact of patients with a large number of events.

An additional analysis of the primary endpoint was included a time-dependent covariate for SGLT-2 inhibitor use.

As a sensitivity analysis for the total HF events endpoint, a joint frailty model with constant baseline hazard for CV death and constant baseline intensity for HF events has been fitted including effects for treatment group, pooled region for stratified analyses and baseline LVEF (<60%, ≥60%). A gamma frailty distribution has been assumed. This model gave a treatment effect on total HF events which was adjusted for a potential treatment effect on CV death.

As supportive analysis for the total HF events endpoint, stratified Cox proportional hazard regression analyses was performed for the following endpoints and plots of Aalen-Johansen estimates of the cumulative incidence functions was provided:
Time to first HF event
Time to first HHF
Time to first urgent HF visit

The additional analyses of the secondary time-to-first event endpoints included an “on treatment analysis” as described above for the primary endpoint.

The proportional hazards assumption was investigated by including a time-treatment interaction into the model and plotting smoothed Schoenfeld residuals. For the renal endpoint, a time-to-first event analysis was done separately for each of the components.

A supportive analysis of the KCCQ TSS has applied a worst-case imputation for death which means that if a patient died, a worst score of 0 for the TSS was imputed for all subsequent visits after the patient's death. Treatment effects at Month 6, 9 and 12 has also been investigated individually by adding a treatment-by-visit interaction into the model.

A responder analysis for the KCCQ TSS was also performed, defining patients with an increase of ≥5 points from baseline to Month 12 (or, for those with a baseline score of >95, a score of >95 at Month 12 without decline from baseline) as a responder. All observed values has been included irrespective of any permanent treatment discontinuation. In case of missing data, a patient's last available post-baseline score prior to Month 12 has been used unless the patient died before Month 12 in which case they were imputed as a non-responder.

Responder status has been analyzed using a logistic regression model including treatment, baseline TSS and stratification factors as covariates; the odds ratio and associated 95% CI has been reported. This analysis was intended to be repeated for cut-offs of ≥10 points increase from baseline to Month 12 (or maintaining a score of >90 from baseline to Month 12 without decrease from baseline) and ≥20 points increase (or maintaining a score of >80 without decrease from baseline). These cut-offs correspond to small (≥5), moderate (≥10) and large (>20) clinically meaningful improvements (Spertus et al, Am Heart J. 2005 October; 150 (4): 707-15. https://doi.org/10.1016/j.ahj.2004.12.010). A further analysis will define those responders who do not experience a ≥5 points decrease from baseline (or, for those with a baseline score of <5, a score of ≥5 at Month 12). This is equivalent to not experiencing a small deterioration. The number and percentage of patients who are responders or non-responders per each of the above criteria has been presented at Months 6, 9 and 12.

A second responder analysis for the KCCQ TSS has used the thresholds derived from the anchor-based analyses with the Patient Global Impression of Severity (PGIS) and Patient Global Impression of Change (PGIC) for a clinically meaningful within-patient change in KCCQ TSS at month 6, 9 and 12, respectively, which were performed separately on blinded data. The specific thresholds has been derived separately for each timepoint, and hence differed for each timepoint.

### (3) Study Interventions

Study intervention is defined as any investigational intervention(s), marketed product(s), placebo, or medical device(s) intended to be administered to a study participant according to the study protocol.

### (4) Safety

Safety assessments includes adverse events (AEs), serious adverse events (SAEs), vital signs including heart rate and blood pressure assessment. Safety laboratory tests includes blood chemistry, hematology and urinalysis.

All safety assessments have been performed in the safety analysis set.

The following safety procedures and variables have been assessed during the study:
SAEs and AEs leading to discontinuation of treatment with study intervention;
Change in body weight from baseline;
Change in serum potassium from baseline;
Number of participants with hyperkalemia (serum potassium >5.5 mmol/L);
Number of participants with severe hyperkalemia (serum potassium >6.0 mmol/L);
Number of participants with hospitalization for hyperkalemia;
Number of participants permanently discontinuing study intervention due to Hyperkalemia;
Change in vital signs (heart rate, SBP and DBP) from baseline;
Change in renal function measured by eGFR (CKD-EPI formula) change from baseline;
Number of participants with hospitalization for worsening of renal function;
Number of participants permanently discontinuing study intervention due to worsening of renal function; and
Changes in laboratory values from baseline.

AEs that occurred or worsened after the first dose of study drug and up to 3 days after the last dose of study drug have been considered as treatment-emergent AEs (TEAEs).

An overall summary of all AEs and treatment emergent AEs (TEAEs) has been generated by treatment group.

The number and percentage of patients with TEAEs, post-treatment AEs occurring more than 3 days after last intake of study intervention, treatment-emergent SAEs, treatment-emergent study intervention-related AEs, treatment-emergent study intervention-related SAEs, TEAEs causing premature and permanent discontinuation of study intervention, treatment-emergent non-serious AEs, TEAEs by maximum intensity, drug-related TEAEs by maximum intensity has been summarized by treatment group using Medical Dictionary for Regulatory Activity (MedDRA) terms grouped by Primary System Organ Class and Preferred Term.

The number of patients with treatment-emergent (until 3 days after last study intervention administration) abnormal laboratory values above or below the normal range has been tabulated by the laboratory parameter and treatment group.

Summary statistics including changes from baseline have been calculated by treatment group and visit for all quantitative laboratory parameters, i.e. for hematology, clinical chemistry and urinalysis. Geometric statistics and ratios to baseline has been presented for urinary albumin-to-creatinine ratio (UACR), creatinine, and NT-proBNP, instead of arithmetic statistics with changes from baseline. For eGFR the relative change has been displayed in addition to the absolute change from baseline.

Summary statistics for serum potassium, eGFR, and serum creatinine have also been repeated by treatment group and visit separately for each level of the stratification factors (region and LVEF).

The following special safety parameters have been further assessed by displaying the number and percentage of patients with safety events as described below by treatment group, visit, and for any time on treatment (including unscheduled assessments) and until 3 days after last study intervention administration. This has also been performed by stratification factors. The summaries have been provided for the number and percentage of patients with:
Absolute value of serum potassium >5.0 mmol/L, >5.5 mmol/L and >6 mmol/L;
Relative decrease from baseline in eGFR of ≥25%, ≥30%, ≥40%, ≥50% and ≥57%;
Absolute value of eGFR <30 mL/min/1.73 m²; and
Increase from baseline in serum creatinine >0.3 mg/dl and >0.5 mg/dL.

### (5) Dosage

Eligible participants received study intervention at the doses illustrated in Table 6 (Table 6: Dosage of Study Intervention for Administration, dispensed as outlined in the SoA (see above Table 1-Schedule of Activities). The dose of finerenone depended on the eGFR value at the Baseline Visit (determined by the local laboratory):
1. Participants with an eGFR≤60 mL/min/1.73 m² started with 10 mg (dose level 1) and had a maintenance dose of 20 mg (dose level 2). Dose level 1 was the minimum dose and dose level 2 was the maximum permitted dose in this group of patients
2. Participants with an eGFR >60 mL/min/1.73 m² started with 20 mg (dose level 2) and had a maintenance dose of 40 mg (dose level 3). Dose level 1 was the minimum dose and dose level 3 was the maximum permitted dose in this group of patients.

The dose could be up-titrated once the participant had been on a stable dose for 4 weeks (±7 days), either at the next regular visit, or at an Up-titration Visit (see Table 6a: Dosage of Study Intervention for Administration). Participants who did not tolerate their starting dose of 20 mg could be down-titrated at any point during the study, including between-scheduled visits if required for safety reasons. These participants could be up-titrated again based on the rules provided in Table 6a or Tables 6b or 6c. If the participant was already at the minimum dose, the study intervention could be interrupted at the investigator's discretion, based on blood potassium levels and renal function which has been monitored throughout the study.

TABLE 6a
Dosage of Study Intervention for Administration

| eGFR value at the Baseline | | |
|---|---|---|
| Visit, based on local | eGFR 25 to ≤60 | eGFR >60 |
| laboratory results: | mL/min/1.73 m² | mL/min/1.73 m² |
| Participant randomized to | Finerenone Placebo | Finerenone Placebo |
| group: | 10 mg finerenone OD Placebo | 20 mg finerenone OD Placebo |
| Starting dose: | OD (Dose Level 1) | OD (Dose Level 2) |
| Maintenance dose: | 20 mg finerenone OD Placebo | 40 mg finerenone OD Placebo |
| Minimum dose after down- | OD (Dose Level 2) | OD (Dose Level 3) |
| titration: | 10 mg finerenone OD Placebo | 10 mg finerenone OD Placebo |
| Maximum dose after up- | OD 20 mg finerenone OD | OD 40 mg finerenone OD |
| titration: | Placebo OD | Placebo OD |

| | |
|---|---|
| Study intervention intake | One tablet of study intervention OD, |
| | preferably in the morning at approximately the same time each |
| | day. |
| | Note: Study intervention could be administered at home, |
| | except on the day of the first PK visit when the tablet was taken |
| | at the study site |
| Missed intake | If discovered within 16 hours after the scheduled time, the |
| | participant should take one tablet of study intervention as soon |
| | as possible |
| | If discovered >16 hours after the scheduled time, this is |
| | considered to be a ‘missed’ dose and the participant had to wait |
| | and take the next tablet of study intervention at the usual |
| | (scheduled) time. |
| Up-titration of dose | Finerenone |
| | Up-titrate study intervention to the next possible higher dose |
| | based on serum/plasma potassium level |
| | eGFR decrease was <30% compared to last scheduled visit. |
| | Placebo |
| | Sham-titrate. |
| Down-titration of dose | If potassium ≥5.5, down-titrate to the next lower dose |
| | level in a step-wise manner (dose level 2 to 1, or dose |
| | level 3 to 2) |
| | If dose level 1 was reached, study intervention treatment had |
| | to be interrupted; |
| | study intervention should be re-introduced at dose level 1 as |
| | soon as the investigator considers it to be medically justified |
| | without compromising safety |
| | If the investigator considered, that the participant could not |
| | tolerate the maximum dose level of study intervention, the |
| | study intervention dose had to be reduced to the next lower |
| | dose level. |

TABLE 6b
Management and Dosing Adjustments in Response
to Changes in Potassium and Renal Function

| | |
|---|---|
| Serum/plasma | |
| potassium | |
| (K⁺ mmol/L) | Action to be taken |

First sample:

| <5.0 | Increase to the next higher dose level (or |
|---|---|
| | continue at maximum permitted dose level) |
| ≥5.0 to <5.5 | Continue the current dose level |
| ≥5.5 to <6.0 | Down-titrate to the next lower dose if |
| | possible; if patient is already on dose |
| | level 1, interrupt study intervention. K⁺ |
| | should be re-checked within 72 h of initial |
| | K⁺ result awareness |
| ≥6.0 | Interrupt study intervention and K⁺ should |
| | be re-checked within 72 h of initial K⁺ |
| | result awareness |

Second and subsequent sample:

| | | |
|---|---|---|
| Option a | <5.5 | Continue current dose |
| | ≥5.5 | Down-titrate to the next lower dose if |
| | | possible, or interrupt study intervention |
| | | and recheck K⁺ |
| Option b | <5.5 | Restart at dose level 1 |
| | ≥5.5 | Continue to withhold study intervention, |
| | | further monitoring of K⁺. Restart at dose |
| | | level 1 ONLY if K⁺ is <5.0 mmol/L |

The following aspects had also to be taken into consideration:
If the participant was already on dose level 1 no further decrease was possible after interruption, the same dose level should be restarted once serum/plasma potassium falls below 5.5 mmol/L. Serum/plasma potassium was to be measured at a safety visit 4 weeks±7 days after re-starting treatment or dose adjustment.
If the participant was on dose level 1 of study intervention, but hyperkalemia recurs soon after a previous event of hyperkalemia leading to interruption of study intervention, and there was no explanation for the recurring hyperkalemia event other than intake of study intervention, premature and permanent discontinuation of study intervention was recommended.
In case of hyperkalemia, it was at the investigator's discretion to take measures, including treatment and monitoring in accordance with local practice standards.

TABLE 6c
Management and Dosing Adjustments in Response
to Changes in Potassium and Renal Function

| eGFR (mL/min/ | |
|---|---|
| 1.73 m²) at | |
| any time after | |
| randomization | Action to be taken |
| Decrease ≥25% | Check for potential reversible causes: |
| and <40% from | Concomitant medications known to affect renal function (e.g. |
| baseline | NSAIDs, antibiotics) |
| | Adverse event (e.g. urinary infection, urinary retention, |
| | dehydration) |
| | Address potential reversible causes if considered clinically |
| | appropriate |
| Decrease ≥40% from | Check for potential reversible causes and address, as above. |
| baseline | At the investigator's discretion, study drug could be |
| | down-titrated or interrupted as follows: |
| | Further monitor eGFR/creatinine |
| | If eGFR/creatinine had reached acceptable levels (to be |
| | determined for the individual participant), re-start study |
| | intervention at the next lower dose level (or dose level 1 |
| | if the participant was already on this dose). |
| | Re-test at central laboratory after 4 weeks to confirm eGFR |
| | decrease of ≥50% or ≥57%* |

Abbreviations: eGFR = estimated glomerular filtration rate; NSAID = non-steroidal anti-inflammatory drug;
*Decrease based on central laboratory data.

### (6) Outcomes:

TABLE 7
The primary outcome of Example 2 was the number
of CV deaths and HF events up to Month 42

| Primary outcome | Secondary outcomes | |
|---|---|---|
| Number of CV deaths and | Time to total HF events | Time to first occurrence of the |
| HF events up to month 42 | (first and recurrent) | composite kidney endpoint: |
| HF events are: | Improvement in NYHA class | Sustained decrease in |
| First and recurrent | from baseline to Month 12 | eGFR ≥50% relative to |
| events | Change in KCCQ-TSS | baseline for ≥4 weeks |
| HHF or urgent care visit | (from baseline to | Sustained eGFR decline <15 |
| for HF | Months 6, 9 and 12) | mL/min/1.73 m² |
| | | Initiation of dialysis or |
| | | kidney transplantation |
| | | Time to death from |
| | | any cause |

The following exploratory endpoints were planned to be analyzed:
Time to first CV hospitalization;
Total number of CV hospitalizations;
Time to first all-cause hospitalization;
Total number of all-cause hospitalizations;
Time to first occurrence of the following composite endpoint: CV death or non-fatal CV event (i.e., non-fatal myocardial infarction, non-fatal stroke, or HHF);
Time to first occurrence of the following composite endpoint: sustained decrease in eGFR ≥57% relative to baseline over at least 4 weeks, or sustained eGFR decline <15 mL/min/1.73 m² or initiation of dialysis or renal transplantation;
Change in eGFR from baseline;
Mean rate of change in eGFR slope and its subcomponents acute and chronic slope;
Change in UACR from baseline;
Days alive and out of hospital (DAOH);
Time to new onset of atrial fibrillation; and
Change in health-related quality of life summary scores from baseline measured by KCCQ and EuroQol Group 5-dimension 5-level questionnaire (EQ-5D-5L), Patient Global Impression of Change (PGIC) and Severity (PGIS).
   Further Endpoints could be:
Clinical endpoints of interest;
Laboratory-based endpoints of interest; and
Breakdown of endpoints;

### Clinical Endpoints of Interest

In addition to the efficacy and safety variables described herein, the effect of finerenone on the following endpoints were intended to be explored. These events that were imbalanced between arms could be analyzed as time-to-event to better understand the time course of event accrual.
Post-randomization signs and symptoms of HF and an expanded composite outcome of CV death, worsening HF event as defined herein or signs and symptoms of HF.
Cardio-renal composite endpoint which included components of the primary endpoint and components of the prespecified secondary renal endpoint.
Renal composite endpoint and cardiorenal composite endpoints analyzed using win statistics (hierarchies to be defined before analysis).
Achievement of post-randomization blood pressure control in those with blood pressure above established thresholds at baseline, including those with apparent treatment resistant hypertension.
Post-randomization changes in number of blood pressure lowering therapies.
New initiation, discontinuation, or dose changes of diuretics.
New initiation, discontinuation, or dose changes of ACE inhibitor/ARB/ARNI.
New initiation or discontinuation of SGLT2 inhibitors.
New initiation or discontinuation of potassium lowering therapies.
New initiation or discontinuation of potassium supplementation.
Post-randomization open-label use and dosing of mineralocorticoid receptor antagonists (including spironolactone, eplerenone, potassium canrenoate, finerenone).
First and total investigator-reported fatal or non-fatal myocardial infarction.
First and total investigator-reported fatal or non-fatal stroke.
Cardiac ischemic events including myocardial infarction, unstable angina, unplanned coronary revascularization, and stroke (individually and as a composite).
Major adverse cardiac events defined either as i) a composite of CV death, non-fatal myocardial infarction, or non-fatal stroke or ii) a composite of death from atherosclerotic CV disease, non-fatal myocardial infarction, or non-fatal stroke.
Proportion of patients with clinically meaningful deterioration (5 point or greater worsening), and small (≥5 point), moderate (≥10 point) and large (≥15 point) improvement in KCCQ-TSS is prespecified herein. Similar responder analyses were performed for KCCQ-CSS, OSS, PL, QOL and Social Limitations Scores.
Analysis of individual domains and questions of the KCCQ.
In addition to assessing improvement in NYHA class as prespecified herein, changes in NYHA class were analyzed on an ordinal basis.
Proportion of patients who become NYHA functional class I post-randomization,
Analysis of days alive and out of hospital/days lost due to death or hospitalization adjusted for quality of life using KCCQ-OSS, EQ-5D-5L, and NYHA.
Outcomes related to new onset AF/AFL and total number of episodes of AF/AFL.
Ventricular arrhythmias, defibrillator discharges, resuscitated cardiac arrest, or sudden death.
Post-randomization HF hospitalizations based on treatment complexity (e.g., standard intravenous diuretics, other intravenous HF therapies, mechanical fluid removal, invasive or non-invasive ventilation, mechanical circulatory support) and treatment course (e.g., intensive care unit requirement, length of stay).

### Laboratory-Based Endpoints of Interest

The effect of finerenone was evaluated for all laboratory measures of interest using each as a continuous variable. The following laboratory-based endpoints were assessed in addition to the analyses described herein:

eGFR
The composite renal endpoint as a secondary endpoint is defined as sustained decrease in eGFR ≥50% relative to baseline over at least 4 weeks, or sustained eGFR decline <15 mL/min/1.73 m² or initiation of dialysis or renal transplantation. Herein, it prespecified analyses of sustained declines in eGFR declines reaching ≥50% and ≥57% thresholds from baseline. Lower thresholds (≥30% and ≥40%) for sustained decreases in eGFR were considered. In addition, a blanking period were included to account for acute, expected eGFR changes.
Focused examination of the “eGFR dip”, the acute changes in eGFR at 1-month and 3-months after randomization
Recalculation of eGFR based on variable calculators (including the 2021 CKD-EPI Equation)
Changes in KDIGO risk categories and transitions in CKD stages post-randomization

### UACR

New-onset macroalbuminuria (among those with normoalbuminuria or microalbuminuria at baseline) or microalbuminuria (among those with normoalbuminuria at baseline)
Regression to normoalbuminuria or microalbuminuria (among those with macroalbuminuria at baseline)

### Potassium

New onset hypokalemia (<3.5 mmol/L)
Assessment of mortality and cardiovascular events for any given potassium level in both arms

### Sodium

Development of hypo- and hypernatremia during follow up and resolution of hypo-and hypernatremia during follow-up. Effect of treatment on sodium as a continuous variable was additionally examined, as the association between baseline levels (categories, continuous) and clinical outcomes.

### Chloride

Development of hypo- and hyperchloremia during follow up and resolution of hypo-and hyperchloremia during follow-up. Effect of treatment on chloride as a continuous variable was additionally examined, as will the association between baseline levels (categories, continuous) and clinical outcomes.

### Uric Acid

Change in uric acid level with finerenone treatment and incidence of gout. Association between uric acid level at baseline (categories, continuous) and clinical outcomes.

### Hematologic Biomarkers

Change in hemoglobin and hematocrit with finerenone treatment and incidence of anemia/resolution of anemia. Association between hemoglobin and hematocrit levels at baseline (categories, continuous), and anemia, and clinical outcomes.
Change in neutrophil/lymphocyte ratio with finerenone treatment. Association between neutrophil/lymphocyte ratio at baseline (categories, continuous) and clinical outcomes.

### Cardiac Biomarkers

Changes in NT-proBNP at 3 months and 12 months and proportion of patients with NT-proBNP levels <1,000 pg/mL post-randomization
Changes in high-sensitivity troponin at 3 months and 12 months and proportion of patients with levels in the normal reference range post-randomization

### Renin and Aldosterone Levels

Change in renin and aldosterone levels and aldosterone renin ratio with finerenone treatment
Association between renin and aldosterone levels and aldosterone renin ratio at baseline and clinical outcomes
Assessment of proportion of patients with evidence of primary hyperaldosteronism based on renin and aldosterone levels

Other biomarkers were analyzed at baseline, Month 3, Month 12, and during the end of study visit. Finerenone's treatment effects on these biomarkers over time and the association between these biomarkers at baseline (categories, continuous) were assessed.
Biomarkers of fibrosis including but not limited to N-terminal propeptide of collagen I and III, tissue inhibitor of matrix metalloproteinase 1, carboxyl-terminal telopeptide of collagen type I, and soluble ST2
Growth/differentiation factor-15
Cancer antigen 125
High sensitivity C-reactive protein

In addition to conventional cardiac biomarkers (e.g., natriuretic peptides and troponins), FINEARTS-HF has prospectively collected exploratory biomarkers that relate to its unique mode of action. These biomarker profiles may help to further distinguish finerenone's mechanistic effects compared with those of conventional MR antagonists.

### Breakdown of Endpoints

Mode of death (including sudden death, worsening HF death, other CV death, non-CV deaths)
Reasons for hospitalization (non-CV hospitalization, HF-related hospitalization, and other CV hospitalizations)
Breakdown of worsening HF events (including urgent visits/Emergency Department stays/oral loop diuretic escalation)
The primary endpoint of CV death and total HF events and the endpoint of CV death alone will be considered with and without incorporation of unknown/undetermined deaths. In a prespecified exploratory analysis, a probabilistic model was applied (predetermined prior to database lock) to better distinguish unknown deaths as either CV or non-CV in etiology. This probabilistic model was built based on known clinical factors that differentially predict adjudicated known cases of CV vs. non-CV deaths.

### (7) Baseline Characteristis

In total 6,014 participants were recruited. The baseline characteristics of these patients were shown in Table 8, Table 9 and Table 10 below.

TABLE 8a
All Example 2 Participants (n = 6,014)

| | | | |
|---|---|---|---|
| Age (years) | 72 ± 10 | Heart Rate (beats/min) | 71 ± 12 |
| Female Sex | 45% | Systolic Blood Pressure (mmHg) | 129 ± 15 |
| BMI (kg/m²) | 30 ± 6 | NT-proBNP (ng/L) (median) | 1,502 ¹⁾ |
| | | | 1,041 ²⁾ |
| Race | | eGFR (mL/min/1.73 m²) | 62 ± 20 |
| Asian | 17% | eGFR ≥60 | 52% |
| Black | 2% | Prior HF Hospitalization | 60% |
| Other | 3% | History of LVEF ≤40% | 5% |
| White | 79% | History of Diabetes | 41% |
| Asia | 16% | History of Atrial Flutter/Fibrillation | 55% |
| Eastern Europe | 44% | History of Hypertension | 89% |
| Latin America | 11% | History of Myocardial Infarction | 26% |
| North America | 8% | History of Stroke | 14% |
| Western Europe, | 21% | Loop Diuretic | 98% |
| Oceania and Others | | | |
| NYHA class | | Beta-blocker | 85% |
| II | 69% | Ace Inhibitor (ACEi) | 36% |
| III | 30% | Angiotensin Receptor Blocker (ARB) | 44% |
| IV | 0.7% | Angiotensin Receptor-Neprilysin | 9% |
| | | Inhibitor (ARNI) | |
| KCCQ-TSS | 67 ± 24 | Calcium Channel Blockers | 33% |
| LVEF (%) | 53 ± 8 | Sodium-glucose Cotransporter-2 | 14% |
| | | Inhibitor (SGLT-2i) | |

The baseline mean LVEF was 53±8% (range, 34 to 84%); 36% of participants had a LVEF <50% and 64% had a LVEF ≥50%. The median NT-proBNP was 1,500 [527, 2962] ¹⁾ and 1,041 [449-1946] ²⁾ pg/ml. 1219 (20%) patients were enrolled during or within 7 days of a worsening HF event, and 3,247 (54%) patients were enrolled within 3 months of a worsening HF event. Compared with prior large-scale HFmrEF/HFpEF trials, FINEARTS-HF participants were more likely to have recent (within 6 months) HF hospitalization, higher NT-proBNP levels, and greater symptoms and functional limitations. Further, concomitant medications included a larger percentage of sodium-glucose co-transporter 2 inhibitors and angiotensin receptor-neprilysin inhibitors than previous trials.

Definition of ¹⁾ and ²⁾ may be found in Example 1b.

TABLE 8b
Further Example 2 Participants - Baseline characteristics

p-value

| | |
|---|---|
| LVEF (%) | Mean value± |
| KCCQ-TSS | Mean value± |
| eGFR (mL/min/1.73 m²) | Mean value± |
| NT-proBNP (ng/L) (median) | Mean value± |
| Baseline UACR mg/g | Mean value± |
| Baseline UACR category, mg/g | |
| <30 | In % |
| 30-299 | In % |
| ≥300 | In % |
| Potassium level (mmol/L) | Mean value± |
| Baseline heart rate, beats/min | Mean value± |
| Baseline systolic blood pressure, mmHg | Mean value± |

TABLE 9a
Baseline Characteristics by Ejection Fraction (The values may be rounded.)

| | LVEF <50 | LVEF ≥50 to <60 | LVEF ≥60 | |
|---|---|---|---|---|
| | (n = 2,179) | (n = 2,659) | (n = 1,145) | P-value |
| Age | 70 ± 10 | 73 ± 9 | 74 ± 9 | <0.001 |
| Female Sex | 31% | 51% | 59% | <0.001 |
| LVEF (%) | 44 ± 3 | 54 ± 3 | 64 ± 5 | <0.001 |
| NYHA class | | | | 0.31 |
| II | 69% | 68% | 71% | |
| III | 31% | 31% | 28% | |
| IV | 0.6% | 0.9% | 0.5% | |
| KCCQ-TSS | 69 ± 24 | 66 ± 24 | 66 ± 24 | <0.001 |
| eGFR (mL/min/1.73 | 65 ± 20 | 61 ± 19 | 60 ± 19 | <0.001 |
| m²) | | | | |
| NT-proBNP (ng/L) | 1,661¹⁾ and | 1,470¹⁾ and | 1,305¹⁾ and | <0.001 |
| (median) | 1,139²⁾ | 1,008²⁾ | 941²⁾ | |
| History of Atrial | 50% | 59% | 55% | <0.001 |
| Flutter/Fibrillation | | | | |
| History of Diabetes | 40% | 41% | 41% | 0.72 |
| Prior HF | 67% | 59% | 51% | <0.001 |
| Hospitalization | | | | |
| History of LVEF ≤40% | 9% | 3% | 1% | <0.001 |

Definition of ¹⁾ and ²⁾ may be found in Example 1b.

TABLE 9b
Baseline Characteristics by Ejection Fraction (The values may be rounded.)

| | Baseline LVEF Group | | | | | | |
|---|---|---|---|---|---|---|---|
| | <50% | | 51-59% | | ≥60% | | P |
| Characteristic | (n = 2,172) | | (n = 2,674) | | (n = 1,147) | | value |
| Age, y | 69.6 ± 10.1 | | 73.3 ± 9.1 | | 73.5 ± 9.2 | | <0.001 |
| Women | 679 | (31.3%) | 1,367 | (51.1%) | 679 | (59.2%) | <0.001 |
| Race | | | | | | | <0.001 |
| Asian | 432 | (20.0%) | 359 | (13.5%) | 205 | (17.9%) | |
| Black | 23 | (1.1%) | 36 | (1.3%) | 29 | (2.5%) | |
| Other | 50 | (2.3%) | 88 | (3.3%) | 27 | (2.4%) | |
| White | 1,659 | (76.7%) | 2,185 | (81.9%) | 883 | (77.2%) | |
| Geographic region | | | | | | | <0.001 |
| Asia | 429 | (19.8%) | 355 | (13.3%) | 199 | (17.3%) | |
| Eastern Europe | 1,007 | (46.4%) | 1,140 | (42.6%) | 503 | (43.9%) | |
| Latin America | 255 | (11.7%) | 281 | (10.5%) | 105 | (9.2%) | |
| North America | 125 | (5.8%) | 218 | (8.2%) | 123 | (10.7%) | |
| Western Europe, | 356 | (16.4%) | 680 | (25.4%) | 217 | (18.9%) | |
| Oceania, Others | | | | | | | |
| History of diabetes | 859 | (39.5%) | 1,091 | (40.8%) | 470 | (41.0%) | 0.61 |
| History of myocardial | 195 | (9.0%) | 67 | (2.5%) | 9 | (0.8%) | <0.001 |
| infarction | | | | | | | |
| History of LVEF <40% | 803 | (37.0%) | 569 | (21.3%) | 163 | (14.2%) | <0.001 |
| Smoking status | | | | | | | <0.001 |
| Current | 235 | (10.8%) | 188 | (7.0%) | 84 | (7.3%) | |
| Former | 713 | (32.8%) | 748 | (28.0%) | 331 | (28.9%) | |
| Never | 1,224 | (56.4%) | 1,738 | (65.0%) | 732 | (63.8%) | |
| Baseline body mass | 29.3 ± 5.9 | | 30.3 ± 6.2 | | 30.4 ± 6.2 | | <0.001 |
| index, kg/m² | | | | | | | |
| Body mass index groups, | | | | | | | <0.001 |
| kg/m² | | | | | | | |
| <18.5 (underweight) | 509 | (23.5%) | 515 | (19.3%) | 217 | (19.0%) | |
| 18.5-<25 (normal | 755 | (34.8%) | 873 | (32.8%) | 360 | (31.4%) | |
| weight) | | | | | | | |
| 25-<30 (overweight) | 532 | (24.5%) | 697 | (26.2%) | 314 | (27.4%) | |
| 30-<35 (class I | 30 | (1.4%) | 21 | (0.8%) | 14 | (1.2%) | |
| obesity) | | | | | | | |
| ≥35 (class II-III | 344 | (15.9%) | 558 | (20.9%) | 240 | (21.0%) | |
| obesity) | | | | | | | |
| Any prior HF | 1,449 | (66.7%) | 1,582 | (59.2%) | 581 | (50.7%) | <0.001 |
| hospitalization | | | | | | | |
| Prior HF hospitalization | 1,143 | (52.6%) | 1,246 | (46.6%) | 433 | (37.8%) | <0.001 |
| (≤6 months) | | | | | | | |
| Prior HF hospitalization | 1,216 | (56.0%) | 1,339 | (50.1%) | 467 | (40.7%) | <0.001 |
| (≤1 year) | | | | | | | |
| Recency of worsening | | | | | | | <0.001 |
| HF event* | | | | | | | |
| ≤7 days | 483 | (22.2%) | 549 | (20.5%) | 187 | (16.3%) | |
| >7 days-≤3 months | 792 | (36.5%) | 941 | (35.2%) | 288 | (25.1%) | |
| >3 months or no | 897 | (41.3%) | 1,184 | (44.3%) | 672 | (58.6%) | |
| worsening HF event | | | | | | | |
| KCCQ - Total Symptom | 69.2 ± 23.9 | | 65.9 ± 23.8 | | 65.5 ± 23.9 | | <0.001 |
| Score | | | | | | | |
| NYHA functional class | | | | | | | 0.13 |
| at baseline | | | | | | | |
| II | 1,499 | (69.0%) | 1,827 | (68.3%) | 815 | (71.1%) | |
| III | 661 | (30.4%) | 824 | (30.8%) | 325 | (28.4%) | |
| IV | 12 | (0.6%) | 23 | (0.9%) | 6 | (0.5%) | |
| Baseline LVEF, % | 44.4 ± 2.8 | | 54.2 ± 2.9 | | 64.0 ± 4.6 | | <0.001 |
| Baseline NT-proBNP, | 1,661 [587, | | 1,472 [490, | | 1,301 [502, | | <0.001 |
| pg/mL | 3390] ¹⁾ and | | 2870] ¹⁾ and | | 2636] ¹⁾ and | | |
| | 1,139 [506- | | 1,008 [426- | | 941 [406- | | |
| | 2205] ²⁾ | | 1880] ²⁾ | | 1776] ²⁾ | | |
| Baseline ECG AF | 1,401 | (64.5%) | 1,575 | (58.9%) | 726 | (63.3%) | <0.001 |
| Baseline heart rate, | 72.1 ± 11.8 | | 71.5 ± 11.7 | | 70.4 ± 11.8 | | <0.001 |
| beats/min | | | | | | | |
| Baseline heart rate | 77.4 ± 12.4 | | 76.2 ± 12.3 | | 75.5 ± 12.8 | | 0.024 |
| (AF), beats/min | | | | | | | |
| Baseline heart rate | 69.1 ± 10.4 | | 68.1 ± 10.1 | | 67.4 ± 10.1 | | <0.001 |
| (non-AF), beats/min | | | | | | | |
| Baseline systolic blood | 127.5 ± 14.9 | | 130.2 ± 15.5 | | 131.2 ± 15.3 | | <0.001 |
| pressure, mmHg | | | | | | | |
| Baseline eGFR, | 64.8 ± 20.1 | | 61.0 ± 19.3 | | 59.6 ± 19.4 | | <0.001 |
| mL/min/1.73 m² | | | | | | | |
| Baseline eGFR ≥60 | 1,243 | (57.2%) | 1,330 | (49.7%) | 537 | (46.8%) | <0.001 |
| mL/min/1.73 m² | | | | | | | |
| Baseline Potassium | 4.42 ± 0.49 | | 4.35 ± 0.47 | | 4.34 ± 0.44 | | <0.001 |
| (mmol/L) | | | | | | | |
| Baseline UACR | | | | | | | 0.98 |
| category, mg/g | | | | | | | |
| <30 | 619 | (29.4%) | 753 | (29.4%) | 338 | (30.2%) | |
| 30-299 | 1274 | (60.5%) | 1559 | (60.8%) | 674 | (60.2%) | |
| ≥300 | 213 | (10.1%) | 253 | (9.9%) | 108 | (9.6%) | |
| Baseline pharmacotherapy | | | | | | | |
| β-blocker | 1,918 | (88.3%) | 2,243 | (83.9%) | 926 | (80.7%) | <0.001 |
| ACEi | 870 | (40.1%) | 890 | (33.3%) | 394 | (34.4%) | <0.001 |
| ARB | 957 | (44.1%) | 1,160 | (43.4%) | 495 | (43.2%) | 0.85 |
| ARNI | 340 | (15.7%) | 144 | (5.4%) | 26 | (2.3%) | <0.001 |
| SGLT2i | 335 | (15.4%) | 364 | (13.6%) | 113 | (9.9%) | <0.001 |
| Loop diuretic | 1,965 | (90.5%) | 2,304 | (86.2%) | 941 | (82.0%) | <0.001 |
| CCB | 516 | (23.8%) | 945 | (35.3%) | 502 | (43.8%) | <0.001 |

Definition of ¹⁾ and ²⁾ may be found in Example 1b.
Values are n (%), mean ± SD, or median [IQR]. P value reflects global comparison across subgroups defined by the recency of HF event before randomization.
*Measured relative to randomization.
**: Baseline UACR unavailable in 204 participants, hence percentages are expressed as the number of participants out of 5797.
Abbreviations: ACEi = angiotensin-converting enzyme inhibitor; AF = atrial fibrillation; ARB = angiotensin receptor blocker; ARNI = angiotensin receptor-neprilysin inhibitor; CCB = calcium channel blocker; ECG = electrocardiogram; eGFR = estimated glomerular filtration rate; HF = heart failure; KCCQ = Kansas City Cardiomyopathy Questionnaire; LVEF = left ventricular ejection fraction; NT-proBNP = N-terminal prohormone of B-type natriuretic peptide; NYHA = New York Heart Association; SGLT2i = sodium-glucose co-transporter 2 inhibitor; SR = sinus rhythm; UACR = urinary albumin-to-creatinine ratio.

TABLE 9c
Baseline Characteristics by LVEF Group: A total of 2172 (36%) patients had LVEF ≥40
to <50%, 2674 (45%) patients had LVEF ≥50% to <60%, and 1147 (19%) patients
had LVEF ≥60%. Participants with a higher LVEF were more likely to be women compared
with men. Those with lower LVEF were more likely to have younger age, had a prior
myocardial infarction, a prior history of LVEF <40%, a prior HF hospitalization,
elevated NT-proBNP levels, and be treated with β-blockers, SGLT2 inhibitors, or an ARNI.

| | Baseline LVEF Group | | | | | | |
|---|---|---|---|---|---|---|---|
| | ≥40 to <50% | | ≥50 to <60% | | ≥60% | | P |
| Characteristic | (n = 2,172) | | (n = 2,674) | | (n = 1,147) | | value |
| Age, y | 69.6 ± 10.1 | | 73.3 ± 9.1 | | 73.5 ± 9.2 | | <0.001 |
| Women | 679 | (31.3%) | 1,367 | (51.1%) | 679 | (59.2%) | <0.001 |
| Race | | | | | | | <0.001 |
| Asian | 432 | (20.0%) | 359 | (13.5%) | 205 | (17.9%) | |
| Black | 23 | (1.1%) | 36 | (1.3%) | 29 | (2.5%) | |
| Other | 50 | (2.3%) | 88 | (3.3%) | 27 | (2.4%) | |
| White | 1,659 | (76.7%) | 2,185 | (81.9%) | 883 | (77.2%) | |
| Geographic region | | | | | | | <0.001 |
| Asia | 429 | (19.8%) | 355 | (13.3%) | 199 | (17.3%) | |
| Eastern Europe | 1,007 | (46.4%) | 1,140 | (42.6%) | 503 | (43.9%) | |
| Latin America | 255 | (11.7%) | 281 | (10.5%) | 105 | (9.2%) | |
| North America | 125 | (5.8%) | 218 | (8.2%) | 123 | (10.7%) | |
| Western Europe, Oceania, Others | 356 | (16.4%) | 680 | (25.4%) | 217 | (18.9%) | |
| History of chronic obstructive | 265 | (12.2%) | 346 | (12.9%) | 158 | (13.8%) | 0.42 |
| pulmonary disease | | | | | | | |
| History of type 2 diabetes | 866 | (39.9%) | 1,096 | (41.0%) | 472 | (41.2%) | 0.67 |
| History of hypertension | 1,858 | (85.5%) | 2,411 | (90.2%) | 1,046 | (91.2%) | <0.001 |
| History of myocardial infarction | 804 | (37.0%) | 569 | (21.3%) | 163 | (14.2%) | <0.001 |
| History of stroke | 308 | (14.2%) | 372 | (13.9%) | 146 | (12.7%) | 0.50 |
| History of LVEF <40% | 195 | (9.0%) | 67 | (2.5%) | 9 | (0.8%) | <0.001 |
| Smoking status | | | | | | | <0.001 |
| Current | 235 | (10.8%) | 188 | (7.0%) | 84 | (7.3%) | |
| Former | 713 | (32.8%) | 748 | (28.0%) | 331 | (28.9%) | |
| Never | 1,224 | (56.4%) | 1,738 | (65.0%) | 732 | (63.8%) | |
| Baseline body mass index, kg/m² | 29.3 ± 5.9 | | 30.3 ± 6.2 | | 30.4 ± 6.2 | | <0.001 |
| Body mass index groups, kg/m² | | | | | | | <0.001 |
| <18.5 (underweight) | 30 | (1.4%) | 21 | (0.8%) | 14 | (1.2%) | |
| 18.5-<25 (normal weight) | 509 | (23.5%) | 515 | (19.3%) | 217 | (19.0%) | |
| 25-<30 (overweight) | 755 | (34.8%) | 874 | (32.8%) | 360 | (31.4%) | |
| 30-<35 (class I obesity) | 532 | (24.5%) | 697 | (26.2%) | 314 | (27.4%) | |
| ≥35 (class II-III obesity) | 344 | (15.9%) | 558 | (20.9%) | 240 | (21.0%) | |
| Any prior HF hospitalization | 1,449 | (66.7%) | 1,582 | (59.2%) | 583 | (50.8%) | <0.001 |
| Prior HF hospitalization (≤6 months) | 1,143 | (52.6%) | 1,247 | (46.6%) | 435 | (37.9%) | <0.001 |
| Prior HF hospitalization (≤1 year) | 1,216 | (56.0%) | 1,339 | (50.1%) | 469 | (40.9%) | <0.001 |
| Recency of index HF event* | | | | | | | <0.001 |
| ≤7 days | 483 | (22.2%) | 549 | (20.5%) | 187 | (16.3%) | |
| >7 days to ≤3 months | 792 | (36.5%) | 943 | (35.3%) | 290 | (25.3%) | |
| >3 months or no index HF event | 897 | (41.3%) | 1,182 | (44.2%) | 670 | (58.4%) | |
| KCCQ - Total Symptom Score | 69.2 ± 23.9 | | 65.9 ± 23.8 | | 65.5 ± 23.9 | | <0.001 |
| NYHA functional class at baseline | | | | | | | 0.31 |
| II | 1,499 | (69.0%) | 1,827 | (68.3%) | 815 | (71.1%) | |
| III | 661 | (30.4%) | 824 | (30.8%) | 325 | (28.4%) | |
| IV | 12 | (0.6%) | 23 | (0.9%) | 6 | (0.5%) | |
| Baseline LVEF, % | 44.4 ± 2.8 | | 54.2 ± 2.9 | | 64.0 ± 4.6 | | <0.001 |
| Baseline hemoglobin Alc, % | 6.5 ± 1.2 | | 6.4 ± 1.2 | | 6.4 ± 1.2 | | 0.014 |
| Baseline NT-proBNP, pg/mL | 1,661 [587, | | 1,472 [490, | | 1,301 [502, | | <0.001 |
| | 3390] ¹⁾ and | | 2870] ¹⁾ and | | 2636] ¹⁾ and | | |
| | 1,139 [506- | | 1,008 [426- | | 941 [406- | | |
| | 2205] ²⁾ | | 1880] ²⁾ | | 1776] ²⁾ | | |
| Baseline ECG AF | 771 | (35.5%) | 1,099 | (41.1%) | 421 | (36.7%) | <0.001 |
| Baseline heart rate, beats/min | 72.1 ± 11.8 | | 71.5 ± 11.7 | | 70.4 ± 11.8 | | <0.001 |
| Baseline heart rate (AF), beats/min | 77.4 ± 12.4 | | 76.2 ± 12.3 | | 75.5 ± 12.8 | | 0.024 |
| Baseline heart rate (non-AF), | 69.1 ± 10.4 | | 68.1 ± 10.1 | | 67.4 ± 10.1 | | <0.001 |
| beats/min | | | | | | | |
| Baseline systolic blood pressure, | 127.5 ± 14.9 | | 130.2 ± 15.5 | | 131.2 ± 15.3 | | <0.001 |
| mmHg | | | | | | | |
| Baseline eGFR, mL/min/1.73 m² | 64.8 ± 20.1 | | 61.0 ± 19.3 | | 59.6 ± 19.4 | | <0.001 |
| Baseline eGFR <60 mL/min/1.73 m² | 929 | (42.8%) | 1345 | (50.3%) | 610 | (53.2%) | <0.001 |
| Baseline Potassium, mmol/L | 4.4 ± 0.5 | | 4.4 ± 0.5 | | 4.3 ± 0.4 | | <0.001 |
| Baseline UACR, mg/g | 18 | [7, 68] | 19 | [7, 67] | 19 | [7, 62] | 0.82 |
| Baseline UACR category, mg/g** | | | | | | | 0.98 |
| <30 | 1,274 | (60.5%) | 1,559 | (60/8%) | 674 | (60.2%) | |
| 30 to <300 | 619 | (29.4%) | 753 | (29.4%) | 338 | (30.2%) | |
| ≥300 | 213 | (10.1%) | 253 | (9.9%) | 108 | (9.6%) | |
| Baseline pharmacotherapy | | | | | | | |
| β-blocker | 1,919 | (88.4%) | 2,243 | (83.9%) | 927 | (80.8%) | <0.001 |
| ACEi | 870 | (40.1%) | 889 | (33.2%) | 392 | (34.2%) | <0.001 |
| ARB | 955 | (44.0%) | 1,159 | (43.3%) | 492 | (42.9%) | 0.82 |
| ARNI | 339 | (15.6%) | 143 | (5.3%) | 27 | (2.4%) | <0.001 |
| SGLT2i | 335 | (15.4%) | 366 | (13.7%) | 113 | (9.9%) | <0.001 |
| Loop diuretic | 1,973 | (90.8%) | 2,318 | (86.7%) | 943 | (82.2%) | <0.001 |
| CCB | 516 | (23.8%) | 946 | (35.4%) | 504 | (43.9%) | <0.001 |

Definition of ¹⁾ and ²⁾ may be found in Example 1b.
Values are n (%), mean ± SD, or median [IQR]. P value reflects global comparison across subgroups defined by the recency of HF event before randomization.
*Measured relative to randomization.
**Baseline UACR unavailable in 204 participants, hence percentages are expressed as the number of participants out of 5,797.
Abbreviations: ACEi = angiotensin-converting enzyme inhibitor; ARB = angiotensin receptor blocker; ARNI = angiotensin receptor-neprilysin inhibitor; CCB = calcium channel blocker; ECG = electrocardiogram; eGFR = estimated glomerular filtration rate; HF = heart failure; KCCQ = Kansas City Cardiomyopathy Questionnaire; LVEF = left ventricular ejection fraction; NT-proBNP = N-terminal prohormone of B-type natriuretic peptide; NYHA = New York Heart Association; SGLT2i = sodium-glucose co-transporter 2 inhibitor; UACR = urinary albumin-to-creatinine ratio.

TABLE 10a
Baseline Characteristics in Patients Recently Hospitalized

| | | HHF between >7 | HHF >3 Months | |
|---|---|---|---|---|
| | HHF <= 7 Days | days and <=3 | Before | |
| | Before | Months Before | Randomization | |
| | Randomization | Randomization | or no Prior HHF | |
| | (n = 1,220) | (n = 2,033) | (n = 2,761) | P-value |
| Age | 72 ± 10 | 71 ± 10 | 72 ± 9 | <0.001 |
| Female Sex | 48% | 46% | 44% | 0.11 |
| LVEF (%) | 52 ± 8 | 52 ± 7 | 54 ± 8 | <0.001 |
| NYHA class | | | | <0.001 |
| II | 51% | 72% | 75% | |
| III | 47% | 28% | 25% | |
| IV | 1.9% | 0.6% | 0.2% | |
| KCCQ-TSS | 53 ± 24 | 70 ± 23 | 71 ± 22 | <0.001 |
| eGFR (mL/min/1.73 m²) | 60 ± 20 | 63 ± 20 | 62 ± 19 | <0.001 |
| NT-proBNP (ng/L) | 1,790 ¹⁾ | 1,691 ¹⁾ | 1,322 ¹⁾ | <0.001 |
| (median) | and 1,168 ²⁾ | and 1,119 ²⁾ | and 952 ²⁾ | |
| History of Atrial | 61% | 55% | 53% | <0.001 |
| Flutter/Fibrillation | | | | |
| History of Diabetes | 42% | 41% | 40% | 0.50 |
| Prior HF Hospitalization | 88% | 83% | 32% | <0.001 |

Definition of ¹⁾ and ²⁾ may be found in Example 1b.

TABLE 10b
Baseline Characteristics by Recency of Worsening HF Event Before Randomization: Patients had
a mean age of 72 ± 10 years; 46% were women, and 79% were white. 69% of patients were NYHA
class II and 31% NYHA class III or IV. 41% had a history of diabetes and 26% had a history of
myocardial infarction. The mean eGFR was 62 mL/min/1.73 m², and 48% had a baseline eGFR less
than 60 mL/min/1.73 m². Overall, 60% of patients had a prior HF hospitalization. The baseline
LVEF was 53 ± 8% and 271 (4.5%) patients had a history of LVEF <40%. The median baseline
NT-proBNP in the overall randomized trial population was 1,500 [527, 2962] ¹⁾ and 1,041 [449-
1946] ²⁾ pg/mL. Among patients with known AFF, the median NT-proBNP was 2,619 [1687, 4347] ¹⁾
and 1,714 [1152-2807] ²⁾ pg/ml; the median NT-proBNP was 731 [360, 1882] ¹⁾ and 588
[313-1255] ²⁾ pg/mL in patients without AFF. The mean Kansas City Cardiomyopathy Questionnaire
(KCCQ) total symptom score (TSS) was 67 ± 24. The majority of patients (61%) had a urinary-albumin-to-
creatinine ratio (UACR) <30 mg/g, 30% had UACR between 30 and 300 mg/g, and 10% had UACR >300
mg/g. Concomitant medications at baseline included beta-blockers (85%), angiotensin-converting enzyme
inhibitors (ACEi) or angiotensin receptor blockers (ARB) (79%), an angiotensin receptor-neprilysin
inhibitor (ARNI) (9%), sodium-glucose co-transporter 2 (SGLT2) inhibitors (14%), and loop diuretics
(87%). Definition of ¹⁾ and ²⁾ may be found in Example 1b.

| | Recency of Worsening HF Event | | | | | | | | |
|---|---|---|---|---|---|---|---|---|---|
| | All FINEARTS- | | | | >7 days to ≤3 | | >3 months or | | |
| | HF Participants | | ≤7 days | | months | | no event | | P |
| Characteristic | (n = 6,001) | | (n = 1,219) | | (n = 2,028) | | (n = 2,754) | | value* |
| Age, y | 72.0 ± 9.6 | | 72.2 ± 9.7 | | 71.3 ± 10.3 | | 72.4 ± 9.1 | | <0.001 |
| Women | 2,731 | (45.5%) | 583 | (47.8%) | 936 | (46.2%) | 1,212 | (44.0%) | 0.06 |
| Race | | | | | | | | | <0.001 |
| Asian | 996 | (16.6%) | 74 | (6.1%) | 507 | (25.1%) | 415 | (15.1%) | |
| Black | 88 | (1.5%) | 7 | (0.6%) | 34 | (1.7%) | 47 | (1.7%) | |
| Other | 165 | (2.8%) | 29 | (2.4%) | 70 | (3.5%) | 66 | (2.4%) | |
| White | 4,735 | (79.1%) | 1,106 | (91.0%) | 1,412 | (69.8%) | 2,217 | (80.8%) | |
| Geographic region | | | | | | | | | <0.001 |
| Asia | 983 | (16.4%) | 74 | (6.1%) | 505 | (25.0%) | 404 | (14.7%) | |
| Eastern Europe | 2,650 | (44.2%) | 759 | (62.3%) | 665 | (32.8%) | 1,226 | (44.5%) | |
| Latin America | 641 | (10.7%) | 122 | (10.0%) | 297 | (14.7%) | 222 | (8.0%) | |
| North America | 471 | (7.8%) | 16 | (1.3%) | 120 | (5.9%) | 335 | (12.2%) | |
| Western Europe, | 1,256 | (20.9%) | 248 | (20.3%) | 441 | (21.7%) | 567 | (20.6%) | |
| Oceania, Others | | | | | | | | | |
| History of chronic | 770 | (12.8%) | 181 | (14.8%) | 251 | (12.4%) | 338 | (12.3%) | 0.06 |
| obstructive pulmonary | | | | | | | | | |
| disease | | | | | | | | | |
| History of type 2 diabetes | 2,438 | (40.6%) | 510 | (41.8%) | 829 | (40.9%) | 1,099 | (39.9%) | 0.50 |
| History of hypertension | 5,323 | (88.7%) | 1,117 | (91.6%) | 1,761 | (86.8%) | 2,445 | (88.8%) | <0.001 |
| History of myocardial | 1,539 | (25.6%) | 269 | (22.1%) | 435 | (21.4%) | 835 | (30.3%) | <0.001 |
| infarction | | | | | | | | | |
| History of stroke | 827 | (13.8%) | 169 | (13.9%) | 301 | (14.8%) | 357 | (13.0%) | 0.16 |
| History of LVEF <40% | 271 | (4.5%) | 36 | (3.0%) | 96 | (4.7%) | 139 | (5.0%) | 0.012 |
| Smoking status | | | | | | | | | 0.020 |
| Current | 509 | (8.5%) | 104 | (8.5%) | 176 | (8.7%) | 229 | (8.3%) | |
| Former | 1,793 | (29.9%) | 343 | (28.1%) | 567 | (28.0%) | 883 | (32.1%) | |
| Never | 3,699 | (61.6%) | 772 | (63.3%) | 1,285 | (63.4%) | 1,642 | (59.6%) | |
| Baseline body mass | 29.9 ± 6.1 | | 30.5 ± 6.2 | | 29.4 ± 6.3 | | 30.1 ± 5.9 | | <0.001 |
| index, kg/m² | | | | | | | | | |
| Body mass index groups, | | | | | | | | | <0.001 |
| kg/m² | | | | | | | | | |
| <18.5 (underweight) | 65 | (1.1%) | 13 | (1.1%) | 37 | (1.8%) | 15 | (0.5%) | |
| 18.5-<25 (normal | 1,241 | (20.7%) | 222 | (18.3%) | 502 | (24.8%) | 517 | (18.8%) | |
| weight) | | | | | | | | | |
| 25-<30 (overweight) | 1,990 | (33.2%) | 397 | (32.6%) | 646 | (31.9%) | 947 | (34.5%) | |
| 30-<35 (class I obesity) | 1,546 | (25.8%) | 319 | (26.2%) | 489 | (24.1%) | 738 | (26.9%) | |
| ≥35 (class II-III | 1,146 | (19.1%) | 265 | (21.8%) | 351 | (17.3%) | 530 | (19.3%) | |
| obesity) | | | | | | | | | |
| Any prior HF | 3,618 | (60.3%) | 1,065 | (87.4%) | 1,685 | (83.1%) | 868 | (31.5%) | <0.001 |
| hospitalization | | | | | | | | | |
| Prior HF hospitalization | 2,827 | (47.1%) | 1,024 | (84.0%) | 1,623 | (80.0%) | 180 | (6.5%) | <0.001 |
| (≤6 months) | | | | | | | | | |
| Prior HF hospitalization | 3,027 | (50.4%) | 1,034 | (84.8%) | 1,644 | (81.1%) | 349 | (12.7%) | <0.001 |
| (≤1 year) | | | | | | | | | |
| KCCQ - Total Symptom | 67.0 ± 23.9 | | 52.9 ± 23.9 | | 70.2 ± 23.3 | | 71.0 ± 22.0 | | <0.001 |
| Score | | | | | | | | | |
| NYHA functional class at | | | | | | | | | <0.001 |
| baseline | | | | | | | | | |
| II | 4,145 | (69.1%) | 618 | (50.7%) | 1,455 | (71.8%) | 2,072 | (75.2%) | |
| III | 1,814 | (30.2%) | 578 | (47.4%) | 559 | (27.6%) | 677 | (24.6%) | |
| IV | 41 | (0.7%) | 23 | (1.9%) | 13 | (0.6%) | 5 | (0.2%) | |
| Baseline LVEF, % | 52.6 ± 7.8 | | 51.7 ± 7.7 | | 51.8 ± 7.3 | | 53.5 ± 8.1 | | <0.001 |
| Pooled LVEF groups, % | | | | | | | | | <0.001 |
| ≥40 to <50 | 2,172 | (36.2%) | 483 | (39.6%) | 792 | (39.2%) | 897 | (32.6%) | |
| ≥50 to <60 | 2,674 | (44.6%) | 549 | (45.0%) | 943 | (46.6%) | 1,182 | (43.0%) | |
| ≥60 | 1,147 | (19.1%) | 187 | (15.3%) | 290 | (14.3%) | 670 | (24.4%) | |
| Baseline hemoglobin | 6.4 ± 1.2 | | 6.4 ± 1.2 | | 6.4 ± 1.2 | | 6.4 ± 1.2 | | 0.13 |
| Alc, % | | | | | | | | | |
| Baseline NT-proBNP, | 1,500 [527, | | 1,775 [538, | | 1,674 [541, | | 1,324 [515, | | <0.001 |
| pg/mL | 2962] ¹⁾ and | | 3846] ¹⁾ and | | 3292] ¹⁾ and | | 2542] ¹⁾ and | | |
| | 1,041 [449- | | 1,168 [474- | | 1,119 [473- | | 952 [426- | | |
| | 1946] ²⁾ | | 2451] ²⁾ | | 2113] ²⁾ | | 1718] ²⁾ | | |
| Baseline ECG AF | 2,293 | (38.2%) | 534 | (43.8%) | 783 | (38.6%) | 976 | (35.4%) | <0.001 |
| Baseline heart rate, | 71.5 ± 11.8 | | 72.5 ± 11.5 | | 72.0 ± 12.2 | | 70.6 ± 11.6 | | <0.001 |
| beats/min | | | | | | | | | |
| Baseline systolic blood | 129.4 ± 15.3 | | 127.1 ± 13.9 | | 128.6 ± 15.9 | | 131.0 ± 15.3 | | <0.001 |
| pressure, mmHg | | | | | | | | | |
| Baseline eGFR, | 62.1 ± 19.7 | | 60.2 ± 20.0 | | 63.3 ± 20.2 | | 62.1 ± 19.2 | | <0.001 |
| mL/min/1.73 m² | | | | | | | | | |
| Baseline eGFR <60 | 2,888 | (48.1%) | 637 | (52.3%) | 918 | (45.3%) | 1,333 | (48.4%) | <0.001 |
| mL/min/1.73 m² | | | | | | | | | |
| Baseline Potassium, | 4.4 ± 0.5 | | 4.4 ± 0.5 | | 4.4 ± 0.5 | | 4.4 ± 0.4 | | 0.06 |
| mmol/L | | | | | | | | | |
| Baseline UACR, mg/g | 18 | [7, 67] | 19 | [7, 73] | 19 | [7, 72] | 18 | [7, 58] | 0.05 |
| Baseline UACR category, | | | | | | | | | 0.30 |
| mg/g** | | | | | | | | | |
| <30 | 3,511 | (60.6%) | 711 | (60.7%) | 1,152 | (59.1%) | 1,648 | (61.5%) | |
| 30 to <300 | 1,712 | (29.5%) | 347 | (29.6%) | 582 | (29.9%) | 783 | (29.2%) | |
| ≥300 | 574 | (9.9%) | 113 | (9.6%) | 214 | (11.0%) | 247 | (9.2%) | |
| Baseline pharmacotherapy | | | | | | | | | |
| β-blocker | 5,096 | (84.9%) | 1,017 | (83.4%) | 1,701 | (83.9%) | 2,378 | (86.3%) | 0.016 |
| ACEi | 2,154 | (35.9%) | 474 | (38.9%) | 638 | (31.5%) | 1,042 | (37.8%) | <0.001 |
| ARB | 2,610 | (43.5%) | 486 | (39.9%) | 948 | (46.7%) | 1,176 | (42.7%) | <0.001 |
| ARNI | 509 | (8.5%) | 76 | (6.2%) | 251 | (12.4%) | 182 | (6.6%) | <0.001 |
| SGLT2i | 816 | (13.6%) | 188 | (15.4%) | 370 | (18.2%) | 258 | (9.4%) | <0.001 |
| Loop diuretic | 5,240 | (87.3%) | 1,075 | (88.2%) | 1,880 | (92.7%) | 2,285 | (83.0%) | <0.001 |
| CCB | 1,970 | (32.8%) | 427 | (35.0%) | 615 | (30.3%) | 928 | (33.7%) | 0.009 |

Definition of ¹⁾ and ²⁾ may be found in Example 1b.
Values are n (%), mean ± SD, or median [IQR].
*P value reflects global comparison across subgroups defined by recency of HF event before randomization.
**Baseline UACR unavailable in 204 participants, hence percentages are expressed as the number of participants out of 5797.
Abbreviations: ACEi = angiotensin-converting enzyme inhibitor; ARB = angiotensin receptor blocker; ARNI = angiotensin receptor-neprilysin inhibitor; CCB = calcium channel blocker; ECG = electrocardiogram; eGFR = estimated glomerular filtration rate; HF = heart failure; KCCQ = Kansas City Cardiomyopathy Questionnaire; LVEF = left ventricular ejection fraction; NT-proBNP = N-terminal prohormone of B-type natriuretic peptide; NYHA = New York Heart Association; SGLT2i = sodium-glucose co-transporter 2 inhibitor; UACR = urinary albumin-to-creatinine ratio

### (8) Further Subgroup Analyses

Exploratory subgroup analysis has been done for the primary and secondary efficacy variables.

The subgroup analyses included subgroups based on e.g. the stratification factors. The list of key subgroups (in addition to the stratification factors) and other subgroups analyzed is specified below, e.g.:
Subgroups based on stratification factors
Subgroups via demography and other baseline characteristics
Subgroups by baseline characteristics
Subgroups by medical history
SPECIAL POPULATIONS (e.g., Recent Worsening HF Event, Diabetes Status)

Analysis included descriptive statistics, graphical display of estimated treatment effects with 95% confidence intervals (CIs) in a forest plot and a statistical test for interaction.

The stratified analyses mentioned in the sections above was combined countries/regions into pooled regions as follows.
Western Europe and Oceania: Australia, Austria, Denmark, Germany, Israel, Netherlands, New Zealand, United Kingdom
Southwestern Europe: Italy, Portugal, Spain
Central Europe: Czechia, Hungary, Poland, Slovakia
Southeastern Europe: Bulgaria, Greece, Romania, Turkey Northeastern Europe: Finland, Latvia, Lithuania, Russia, Ukraine
Asia: China, Hong Kong, India, Japan, Malaysia, South Korea, Taiwan
North America: Canada, United States of America
Latin America: Argentina, Brazil, Colombia, Mexico

### Subgroups Based on Stratification Factors

Pooled region for subgroup analysis (Western Europe, Oceania and Others; Eastern Europe; Asia; North America; Latin America); the subgroup Western Europe, Oceania and Others combines the groups Western Europe and Oceania and Southwestern Europe from the pooled region for stratified analyses. The subgroup Eastern Europe combined the groups Central Europe, Southeastern Europe, and Northeastern Europe from the pooled region for stratified analyses.
LVEF (<60%, ≥60%)

### Subgroups

Baseline serum potassium value (£ 4.5, >4.5 mmol/L)
eGFR category at baseline (eGFR <60, ≥60 mL/min/1.73 m²)
Atrial fibrillation at baseline electrocardiogram (ECG) (present, absent)
Diabetes Mellitus at baseline (present, absent)
Index HF event (very recent (≤7 days before randomization), recent (>7 days-≤3
months), >3 months or no index HF event)
Race (white, black, Asian, other)
Sex (male, female)
Age (£ median vs. >median)
Baseline body mass index (BMI) (<30 vs. ≥30 kg/m²)
Systolic blood pressure (SBP) at baseline (£ median vs. >median)
Angiotensin-converting enzyme inhibitor (ACEI), angiotensin receptor blocker (ARB) or angiotensin receptor-neprilysin inhibitor (ARNI) use at baseline (yes, no)
Treatment with sodium-glucose transport proteins-2 inhibitor (SGLT-2i) (yes, no)
NYHA functional class at baseline (II, III/IV)
Baseline NT-proBNP (≤median vs. >median)
I Baseline UACR (<30 vs. ≥30 mg/g)

### Subgroups Via Demography and Other Baseline Characteristics

Demography includes age (continuous and categorized by 40-<65, 65-<75, 75-<85, ≥85), sex, race, ethnicity, pooled region (for stratified analysis and for subgroup analysis), body weight (continuous and categorized by <60, 60-<90, ≥90 kg), body height, BMI (continuous and categorized by <30 vs. ≥30 as well as by <18.5, 18.5 to <25, 25 to <30, 30 to <35, ≥35 kg/m²), hip and waist circumference and waist-hip ratio, smoking history (never, former, current smoker) and alcohol consumption.

### Subgroups by Other Baseline Characteristics:

LVEF (continuous and categorized by <60% vs. ≥60% as well as <50, 50-<60, ≥60%)
NYHA Class (II, III, IV)
index HF event (randomized during/at HF event, very recent (≤7 days from randomization), recent (>7 days-≤3 months), >3 months, no index; as well as ≤7 days from randomization, >7 days-≤3 months, >3 months or no index event)
type of (latest) index HF event with respective timing (hospitalization for heart failure, urgent HF visit, no index event)
serum potassium (continuous and categorized by ≤4.5 mmol/L vs. >4.5 mmol/L)
eGFR (calculated by CKD-EPI formula, Japanese formula adjustment made for subjects recruited in Japan; continuous and categorized by <60 vs. ≥60 mL/min/1.73 m² as well as <45, 45 to <60, 60 to <90, ≥90 mL/min/1.73 m²),
serum creatinine (continuous)
SBP (continuous and categorized by 90-<130, 130-<160, ≥160 mmHg)
diastolic blood pressure [DBP] (continuous)
heart rate (continuous)
NT-proBNP (continuous)
UACR (continuous and categorized by <30 vs. ≥30 as well as <30, 30-<300, ≥300 mg/g)
history of LVEF<40% (yes [improved], no). For subjects with a history of LVEF <40%, prior LVEF values will be summarized
Atrial fibrillation at baseline per ECG
Diabetes Mellitus at baseline
ACEI, ARB or ARNI use at baseline
SGLT-2i use at baseline.

### Subgroups by Medical History:

Medical history was coded using the Medical Dictionary for Regulatory Activities (MedDRA). Medical history was presented for each MedDRA Primary System Organ Class (SOC) and Preferred Term (PT) by treatment group. Additional medical history terms by the following Standard MedDRA Queries (SMQs), Project-specific Bayer MedDRA Queries (PBMQs), Bayer MedDRA Labeling Groupings (MLGs) or selected PTs will also be presented:

Hyperlipidemia (MLG)
Hypertension (MLG)
Type 2 diabetes mellitus (PT)
Atrial fibrillation/flutter (PBMQ)
Ischemic Stroke/Transitory Ischemic Attack (TIA) (PBMQ)
Myocardial Infarction (MLG)
Coronary Artery Disease (PBMQ)
Peripheral Arterial Occlusive Disease (PBMQ)
Cardiac Failure (MLG)
Coronary Artery Bypass Graft (CABG) (PBMQ)
Chronic Obstructive Pulmonary Disease (COPD) (PT)
Percutaneous coronary intervention (PCI) (PBMQ)
COVID-19 (SMQ narrow)
Liver cirrhosis (PT)
Sleep Apnea Syndrome (PT)
Chronic Kidney Disease (CKD) (PT)

### Subgroups by Concomitant Therapy:

Concomitant medications was coded using the World Health Organization Drug Dictionary (WHO-DD). The number of subjects who took at least one concomitant medication, the number of subjects who took at least one medication that started before administration of study drug and the number of subjects who took at least one concomitant medication that started after start of study drug and the number of subjects who took at least one medication ongoing at baseline (i.e., starting before or on the day of randomization and ending at least one day after the day of randomization) drug was presented by treatment group and overall using anatomical therapeutic chemical (ATC) classes and subclasses. These tables were repeated summarizing the number of subjects with medications in the standard drug groups of interest.
ACEIs and ARBs
ARNI
Beta-blockers
Loop diuretics
Thiazide diuretics
Digoxin
Nitrates
Potassium supplements
Potassium lowering agents (including binders)
Alpha blocking agents
Calcium channel blockers
Centrally acting antihypertensives
Strong, unclassified, moderate, weak cytochrome P450 isoenzyme 3A4 (CYP3A4)
inhibitors and CYP3A4 inducers
Aspirin
Statins
History of prior MRA use (yes, no)
Organic anion transporting polypeptides (OATP) substrates

### Anti-Diabetic Drugs

Insulin and analogues
SGLT-2 inhibitors
Other anti-diabetic drugs (Dipeptidyl Peptidase 4 inhibitors or Glucagon-like peptide-1 agonists or Biguanides or Sulfonylureas or Alpha glucosidase inhibitors or Metiglinides or Thiazolidinediones.)

### Special Populations

### Recent Worsening HF Event:

Example 2 was specifically enriched to examine the population of patients with recent worsening HF. Depending on the analysis, subpopulations of interest could be considered including randomization at the time of a hospitalization for HF or urgent HF visit, within 7 days of worsening HF, and within 3 months of worsening HF.
Among those who were hospitalized at the time of randomization, 30-, 60-, and 90-day readmissions (all-cause, HF-related, and CV-related) will be examined
Early changes in diuretic use and dosing
Early trajectory of health status and quality of life
Early safety and tolerability with particular attention to blood pressure and renal function changes
No prior trial of HFmrEF/HFpEF had specifically targeted those with recent worsening heart failure event, a strategy of enrichment that had been employed in HFrEF. In Example 2, the proportion of patients without recent worsening HF event in the 3 months prior to enrollment were prospectively planned to account for approximately half of the trial population. Indeed, 54% of all participants in Example 2 had a worsening heart failure within 3 months of enrollment. This resulted in a somewhat higher risk population than seen in prior trials, as evidenced by the highest natriuretic peptide levels and highest symptom burden (based on the lowest baseline KCCQ-total symptom score). Participants with recent worsening heart failure events were anticipated to face relatively higher event rates. In addition, 12% of Example 2 participants were enrolled during an episode of worsening HF. Recent data have suggested that rapid implementation of guideline-directed medical therapies including MRAs appears safe and highly effective after a hospitalization for HF. FINEARTS-HF added placebo-controlled prospective data informing the safety and efficacy of in-hospital initiation of non-steroidal MRAs.
Recency of HF Events Prior to Randomization in Example 2: The Example 2 trial population could be segmented according to timing from most recent HF event (HF hospitalization or urgent HF visit) prior to trial enrollment: 749 (12%) [during index HF event], 470 (8%) [equal to or below 7 days], 2,024 (34%) [above 7 days to 3 months], 937 (16%) [above 3 months], 1,821 (30%) [no index HF event].

### Diabetes Status:

As finerenone was previously studied in patients with chronic kidney disease exclusively with comorbid diabetes, Example 2 provides the largest examination to date regarding the use of finerenone in patients without diabetes.
Treatment effects on primary and secondary endpoints will be assessed by glycemic categories (no diabetes, prediabetes, and T2D), across HbA1c as a continuous measure, and across various background anti-hyperglycemic therapies including insulin
In the T2D subgroup, new anti-hyperglycemic therapy initiation and changes in insulin dose (in those on insulin at baseline) will be examined
In the T2D subgroup, changes in hemoglobin A1c will be examined
In the T2D subgroup, assessment of the primary and secondary endpoints will be performed by background anti-hyperglycemic therapies including focused examination of patients on various combinations of therapies
In the non-T2D subgroup, new diagnosis of diabetes will be examined
Focused assessment of the renal endpoints as described herein will be performed separately in patients with and without diabetes
Focused assessment of safety and tolerability as described herein will be performed separately in patients with and without diabetes

### Albuminuria:

Example 2 targeted a high-risk population at the intersection of cardio-kidney-metabolism. Despite an average eGFR >60 mL/min/1.73 m², almost 40% of participants had evidence moderately or severely increased levels of albuminuria.

### Further Subgroups:

The following subgroups of interest were explored to examine event rates and for consistency of efficacy and safety of finerenone. All subgroups were identified based on randomization or pre-randomization data unless otherwise specified. For each subgroup, the treatment effect and interaction with treatment for the primary endpoint and each of the secondary endpoints has been assessed, including components of the primary endpoint. For laboratory variables, the association between baseline levels and outcomes has been evaluated, along with the effect of finerenone on these variables.
Potassium subgroups were specified above according to the following cutpoints (≤4.5, >4.5 mmol/L). Additional subgroups defined by median, quantiles, and other standard definitions of hypo-, hyper- and normokalemia were assessed. Potassium was additionally examined as a continuous function.
Sodium subgroups defined by the median, quantiles, and standard definitions of hypo-, hyper-and normonatremia. Sodium was additionally examined as a continuous function.
Chloride subgroups defined by the median, quantiles, and standard definitions of hypo-, hyper-and normochloremia. Chloride was additionally examined as a continuous function.
Hemoglobin and hematocrit subgroups defined by the median, quantiles, and standard definitions of anemia. Hemoglobin and hematocrit were additionally examined as a continuous function.
eGFR subgroups were specified above according to the following cutpoints <60, ≥60 mL/min/1.73 m². eGFR categories were additionally evaluated according the full KDIGO risk categories and treatment effects were examined across eGFR as a continuous function
Focused examination of Stage 4 CKD (if eGFR was less than 30 mL/min/1.73 m² at randomization or at any post-randomization measurement)
UACR subgroups were specified above according to the following cutpoints (<30 vs. ≥30 mg/g). UACR was additionally as a continuous function.
BUN and BUN/creatinine ratio subgroups defined by the median and quantiles.
BUN and BUN/Cr ratio was additionally examined as a continuous function.
Patients suspected to have primary hyperaldosteronism based on renin and aldosterone levels
BMI subgroups were specified above according to the following cutpoints (<30 kg/m²≥30 kg/m²). BMI categories were additionally be evaluated according to the full WHO classification and treatment effects were examined across BMI as a continuous function. Similar analyses were used for other recognized anthropometric measures e.g., waist-to-height ratio, waist-hip ratio, body roundness index, examining these as categorical variables using recognized cut points, medians, quantiles, and as continuous variables.
Subgroups defined by standard nutritional indices e.g., the geriatric nutritional risk index (GNRI), prognostic nutritional index (PNI), and controlling nutritional status (CONUT), examining these as categorical variables using recognized cut points, medians, quantiles, and as continuous variables.
Age subgroups were specified above according to the following cutpoints (median age). Specific evaluation of older age categories were considered and treatment effects was examined across age as a continuous function
Race subgroups were specified above. Focused examination of patients who are Black, Asian, and Native American
Systolic blood pressure subgroups were specified above according to the following cutpoints (median age). SBP and DBP have been assessed at the median, quantiles, and various global definitions of hypertension (based on both SBP and DBP). Treatment effects have been examined across SBP and DBP as a continuous function
Patients with apparent treatment resistant hypertension
Atrial fibrillation on baseline ECG is a prespecified subgroup above. Atrial fibrillation or atrial flutter on baseline ECG has been further examined. In addition, a previous history of atrial fibrillation/flutter has been assessed, together with the overlap between medical history and baseline ECG and derived history of persistent/permanent AF versus paroxysmal AF.
Patients with improved/recovered LVEF (those who had LVEF ≤40% at any time prior to randomization)
LVEF was a stratification variable at the following cutpoint (<60%, ≥60%). Different cutpoints (≤49%, 50 to 59%, ≥60%) were also applied to examine those with HF with mildly reduced ejection fraction. Additional LVEF subgroups to limit digit preference were considered and treatment effects were examined across LVEF as a continuous function. In addition, the two-way interaction between sex and LVEF have been examined.
Other anthropometric indices e.g., waist-to-hip ratio, waist-to-height ratio using quantiles and recognized cutpoints
Time from prior HF hospitalization as a continuous measure
Time from index HF diagnosis
Patients with COPD
Patients with OSA
Patients with cirrhosis
Patients with any atherosclerotic cardiovascular disease
Patients with history of stroke/TIA
Patients with history of coronary artery disease/prior MI
Patients with history of coronary artery bypass graft surgery
Patients with peripheral artery disease
Patients with metabolic syndrome (using standard definitions)
Subgroups based on baseline use and dosing of diuretics
Patients with multimorbidity and frailty
Patients with cardio-renal-metabolic overlap (intersection of atherosclerotic cardiovascular disease, CKD, and diabetes)
Total baseline medications will be categorized as (“non-polypharmacy”: <5 medications; “polypharmacy”: 5 to 9 medications; and “hyperpolypharmacy”: ≥10 medications) and also assessed as a continuous function
Number of blood pressure lowering therapies at baseline
Patients with baseline risk as determined by the MAGGIC and other HF risk scores
Patients with baseline risk as determined by the Kidney Failure Risk Equation
Subgroups based on baseline evidence of congestion and congestion scores
Regional subgroups based on socioeconomic differences based on the GINI coefficient
Subgroups based on KCCQ-TSS and other KCCQ domains at baseline.
Subgroups based on EQ-5D-5L VAS and index scores at baseline.
Time from diagnosis of heart failure, analyzed as both a continuous and a categorical variable
Time from HF hospitalization continuously and focused examination for those never hospitalized
NT-proBNP and high-sensitivity troponin will be examined at median, quantiles, and as continuous measures
History of any prior MRA use prior to randomization
Background HF therapies including focused examination of patients on various combinations of therapies (including the Heart Failure Collaboratory score)
Patients on or off β-blockers at baseline
Patients on or off potassium supplements at baseline
Patients on or off potassium lowering therapies at baseline
Patients with phenotypic characteristics of amyloid cardiomyopathy, based on validated clinical models/scores

The trial according to Example 2 was performed. The following examples describe, e.g., outcomes and subgroups.

### Example 3—Finerenone in Heart Failure with Mildly Reduced or Preserved Ejection Fraction: Design and Baseline Characteristics of the Example 2 Trial

Over 6,000 patients (mean age 72±10 years; 45% women, 41% type 2 diabetes, LVEF 53+8%) from 635 sites across 37 countries were randomized. 12% were enrolled in hospital, 8% were enrolled within 7 days of a HF hospitalization or urgent HF visit, 34% were enrolled between 8 and 90 days of a HF hospitalization or urgent HF visit, and 46% had no recent HF hospitalization or urgent HF visit. 19% had an LVEF≥60%. Comorbidities were common and 54% of patients had a history of atrial fibrillation/flutter. Notable differences in baseline characteristics are discernible among different enrollment subgroups in the Example 2 study and comparative trials. For example, in Example 2, patients enrolled during a hospitalization or within seven days of a worsening HF episode, constituting 48% females and a mean LVEF of 51.7±7.7, represented a comparatively higher risk group. This subgroup demonstrated a higher prevalence of atrial fibrillation both historically (60%) and at baseline (45%), a higher median N-terminal prohormone of brain natriuretic peptide (NT-proBNP) level (1,7971) and 1,168 2)), and greater hypertension incidence (91%) than other subgroups. In a broader comparison, the Example 2 cohort exhibited more severe health status than those in the EMPEROR Preserved and DELIVER trials. This is suggested by a lower proportion of patients in the NYHA class II (69% in Example 2 versus 82% and 75% in EMPEROR Preserved and DELIVER respectively), and higher median NT-proBNP levels (1,509 ¹⁾ and 1,041 ²⁾) in Example 2 compared to 974 in EMPEROR Preserved and 1,011 in t DELIVER trials).

Definition of ¹⁾ and ²⁾ may be found in Example 1b.

Example 2 has enrolled a broad global population of patients with HFmrEF/HFpEF. Over half were enrolled during or within 90 days of an episode of worsening HF. We will present the design and baseline data from Example 2 in the context of other contemporary trials in similar patients.

### Example 4—Finerenone in Heart Failure with Mildly Reduced or Preserved Ejection Fraction: Design and Baseline Characteristics of the Example 2 Trial

Over 6000 patients (mean age 72±10 yrs; 45% women, 41% T2D, LVEF 53±8%; Table 11: Baseline Characteristics of Example 2, Example 2 subgroups, and other recent trials in HFmrEF or HFpEF) from 635 sites across 37 countries were randomized. 12% were enrolled in hospital, 8% were enrolled within 7 days of a HF hospitalization or urgent HF visit, 34% were enrolled between 8 and 90 days of a HF hospitalization or urgent HF visit, and 46% had no recent HF hospitalization or urgent HF visit. 19% had an LVEF≥60%. Comorbidities were common and 54% of patients had a history of atrial fibrillation/flutter at enrollment. Baseline characteristics differed substantially by enrollment subgroup (Table: Baseline Characteristics of Example 2, Example 2 subgroups, and other recent trials in HFmrEF or HFpEF). Participants in Example 2 represent a higher risk cohort with worse functional class and higher natriuretic peptides than in prior trials of HFmrEF/HFpEF (Table 11: Baseline Characteristics of Example 2, Example 2 subgroups, and other recent trials in HFmrEF or HFpEF).

Example 2 has enrolled a broad global population of patients with HHFmrEF/HFpEF. Over half were enrolled during or within 90 days of an episode of worsening HF. The design and baseline data from Example 2 were presented in the context of other contemporary trials in similar patients:

TABLE 11
Baseline Characteristics of Example 2, Example 2 subgroups,
and other recent trials in HFmrEF or HFpEF

| | | Example 2 Subgroups based on | | | | |
|---|---|---|---|---|---|---|
| | | Setting/Timing of Enrollment | | | | |
| | | Enrolled in | Enrolled | | | |
| | | Hospital or | between 8 | | | |
| | | within 7 | and 90 | | | |
| | | days of | days after | No recent | | |
| | Example 2 | worsening | worsening | worsening | EMPEROR | |
| | Overall | HF episode | HF episode | HF episode | Preserved | DELIVER |
| Characteristic | (N = 6,016) | (N = 1,226) | (N = 2,031) | (N = 2,759) | (N = 5,988) | (N = 6,263) |
| Age (mean ± SD) | 72 ± 10 | 72 ± 10 | 71 ± 10 | 72 ± 9 | 72 ± 9 | 72 ± 10 |
| Female | 45% | 48% | 46% | 44% | 45% | 44% |
| LVEF (mean ± SD) | 52.5 ± 7.8 | 51.7 ± 7.7 | 51.8 ± 7.4 | 53.5 ± 8.0 | 54 ± 9 | 54 ± 9 |
| eGFR (mean ± SD) | 62.7 ± 19.9 | 61.1 ± 20.0 | 64.0 ± 20.8 | 62.5 ± 19.1 | 61 ± 20 | 62 ± 19 |
| NT-proBNP | 1509 ¹⁾ and | 1797 ¹⁾ and | 1690 ¹⁾ and | 1331 ¹⁾ and | 974 | 1011 |
| (median) | 1041 ²⁾ | 1168 ²⁾ | 1119 ²⁾ | 952 ²⁾ | | |
| NYHA II | 69% | 51% | 72% | 75% | 82% | 75% |
| History of AF | 54% | 60% | 54% | 52% | 52% | 56% |
| AF at baseline | 39% | 45% | 39% | 36% | 35% | 42% |
| History of | 88% | 91% | 86% | 88% | 90% | 89% |
| Hypertension | | | | | | |
| Type 2 diabetes | 41% | 42% | 41% | 40% | 49% | 45% |
| mellitus | | | | | | |
| History of | 26% | 22% | 21% | 30% | 29% | 26% |
| Myocardial | | | | | | |
| infarction | | | | | | |
| MEDICATIONS | | | | | | |
| Diuretics | 99% | 99% | 99% | 100% | 86% | 98% |
| Beta-blockers | 86% | 86% | 84% | 86% | 86% | 76% |
| Angiotensin | 45% | 43% | 47% | 43% | 39% | 34% |
| Receptor | | | | | | |
| Blockers | | | | | | |
| ACE-inhibitors | 38% | 43% | 34% | 39% | 40% | 33% |
| ARNI | 8.6% | 6.3% | 13% | 7% | 2% | 4% |
| Calcium channel | 33% | 37% | 31% | 32% | 31% | 31% |
| blockers | | | | | | |
| SGLT-2 | 13% | 14% | 18% | 9% | — | — |
| inhibitors | | | | | | |

Definition of ¹⁾ and ²⁾ may be found in Example 1b.

### Example 5—Pain and Anxiety in Heart Failure—Findings from Example 2

Usually, assessment of health status in heart failure is focused on typical symptoms such as dyspnoea and fatigue. However, patients may suffer other physical and psychological consequences of heart failure and associated comorbidities, which can be evaluated using generic health-related quality-of-life instruments such as the EuroQol 5-Dimension questionnaire (EQ-5D-5L). We used the EQ-5D-5L to obtain a broader assessment of health status in patients with heart failure with mildly reduced or preserved ejection fraction (HFmrEF/HFpEF) enrolled in the Example 2.

The five dimensions of the descriptive section of the EQ-5D-5L include mobility (“walking around”), self-care (“washing or dressing self”), usual activities (“e.g., work, study, housework, family or leisure activities”), pain/discomfort (“pain or discomfort”), and anxiety/depression (“anxious or depressed”). Each dimension is divided into 5 levels (1-5) i.e., no problem, slight problem, moderate problem, severe problem, and extreme problem (cannot do activity/extreme symptom). Among 6,001 participants in Example 2, 5,778 (99.6%) completed all EQ-5D-5L questions. Only around a third of patients reported no problem with mobility and carrying out usual activities (and 38% and 33% of patients, respectively, had moderate or greater problems in these dimensions). Overall, only 39% of patients were free of pain/discomfort and half did not report anxiety or depression (although 26% and 19%, respectively, had moderate or greater problems in these dimensions).

Most patients with HFmrEF/HFpEF experience some difficulty related to mobility, undertaking usual activities, and even self-care. More surprisingly, many patients report pain or discomfort, the basis of which is unclear and requires further investigation. Approximately half of patients experienced some anxiety or depression, and this was at least moderately severe in one in five participants; the management of this dimension may be an important unmet need in heart failure. Generic health-related quality-of-life instruments may provide additional and complementary information to that obtained from disease-specific instruments such as KCCQ. Results are summarized in Table 12.

TABLE 12
Distribution of answers to each EQ-5D-5L question at baseline.

| | No | Slight | Moderate | Severe | Unable |
|---|---|---|---|---|---|
| | Problem | Problem | Problem | Problem | to do |
| | [%] | [%] | [%] | [%] | [%] |
| Mobility | 32.7 | 29.1 | 26.8 | 10.6 | 0.8 |
| Self-care | 65 | 20.4 | 11.5 | Less | Less |
| | | | | than 5 | than 5 |
| Usual activities | 35.6 | 31.9 | 23.6 | 6.9 | 3.1 |
| Pain/discomfort | 39.3 | 35.2 | 19.7 | 5.2 | 2 |
| Anxiety/ | 51.4 | 29.5 | 14.3 | Less | Less |
| depression | | | | than 5 | than 5 |

EQ-5D-5L = EuroQol 5-Dimension 5-Level questionnaire.

### Example 6—Generalizability of the Example 2 Trial to the U.S. Population Across the Spectrum of Kidney Risk

To assess generalizability, we conducted serial cross-sectional analyses of US adults from the National Health and Nutrition Examination Surveys (2015-2016, 2017-2018, 2019-March 2020). Samples were weighted to be nationally representative of the US population. We identified adults with HF (age≥40 years with HF, eGFR≥25 mL/min/1.73 m², and potassium≤5.0 mmol/L) in NHANES. In both the enrolled Example 2 and the NHANES HF sample, we examined the distribution of KDIGO risk categories.

Among 6,001 patients enrolled in Example 2, 5,810 (97%) had available eGFR and UACR data. Median baseline eGFR was 61 (IQR 47-77) mL/min/1.73 m² and 2,789 (48%) had an eGFR<60 mL/min/1.73 m². Median baseline UACR was 18 (IQR 7-67) with 2,279 (39%) with UACR≥30 mg/g. In Example 2, 35%, 29%, 20%, and 16% of participants were classified as KDIGO low, moderately increased, high, and very high risk, respectively. Among patients with diabetes, 26%, 27%, 23%, and 24% were KDIGO low, moderately increased, high, and very high risk, respectively. Participants in higher KDIGO risk categories were more likely be older, women, Asian, more functionally impaired with greater symptom burden, were more likely to have a previous HF hospitalization and had higher prevalence of diabetes, and had markedly higher natriuretic peptide levels (Table 13-Baseline Clinical Profiles of Example 2 Trial Participants with Available eGFR and UACR Data). It was estimated that 5,189,186 (4,333,114 to 6,045,257) individuals with HF resided in the U.S. of whom 48%, 32%, 12%, and 9% would be classified as KDIGO low, moderately increased, high, and very high risk, respectively (Table 14-KDIGO Risk Categories—U.S. HF Population in NHANES-(Weighted n=5,189,186 from 2015-2020)).

Example 2 has enrolled a global population of patients with HF with mildly reduced or preserved ejection fraction with a broad spectrum of kidney risk well-represented. Distribution of kidney risk is highly overlapping and generalizable to many patients with HF in the US.

TABLE 13
Baseline Clinical Profiles of Example 2 Trial Participants with Available eGFR and UACR Data
Baseline Clinical Profiles of Example 2 Trial Participants
with Available eGFR and UACR Data (n = 5,810)

| | KDIGO Risk Categories | | | | | | | | |
|---|---|---|---|---|---|---|---|---|---|
| | | | Moderately | | | | Very | | |
| | Low | | Increased | | High | | High | | P- |
| | n = 2,035 | | n = 1,688 | | n = 1,172 | | n = 915 | | value |
| Age | 68.2 ± 9.7 | | 72.2 ± 8.9 | | 75.0 ± 9.0 | | 75.3 ± 8.8 | | <0.001 |
| Women | 835 | (41.0%) | 801 | (47.5%) | 573 | (48.9%) | 421 | (46.0%) | <0.001 |
| Body Mass Index | 29.8 ± 5.9 | | 30.2 ± 6.1 | | 29.6 ± 6.4 | | 30.1 ± 6.3 | | 0.82 |
| (kg/m²) | | | | | | | | | |
| Race | | | | | | | | | <0.001 |
| White | 1,629 | (80.3%) | 1,344 | (79.8%) | 904 | (77.5%) | 680 | (74.4%) | |
| Asian | 318 | (15.7%) | 277 | (16.4%) | 206 | (17.7%) | 190 | (20.8%) | |
| Black | 25 | (1.2%) | 27 | (1.6%) | 18 | (1.5%) | 11 | (1.2%) | |
| Other | 56 | (2.8%) | 37 | (2.2%) | 38 | (3.3%) | 33 | (3.6%) | |
| Region | | | | | | | | | <0.001 |
| Asia | 314 | (15.4%) | 272 | (16.1%) | 206 | (17.6%) | 188 | (20.5%) | |
| Eastern Europe | 1,084 | (53.3%) | 784 | (46.4%) | 444 | (37.9%) | 271 | (29.6%) | |
| Latin America | 218 | (10.7%) | 164 | (9.7%) | 149 | (12.7%) | 97 | (10.6%) | |
| North America | 112 | (5.5%) | 127 | (7.5%) | 101 | (8.6%) | 95 | (10.4%) | |
| Western Europe, | 307 | (15.1%) | 341 | (20.2%) | 272 | (23.2%) | 264 | (28.9%) | |
| Oceania and | | | | | | | | | |
| Others | | | | | | | | | |
| NYHA Class | | | | | | | | | <0.001 |
| NYHA Class II | 1,507 | (74.1%) | 1,175 | (69.6%) | 790 | (67.4%) | 551 | (60.2%) | |
| NYHA Class III | 514 | (25.3%) | 506 | (30.0%) | 372 | (31.7%) | 356 | (38.9%) | |
| NYHA Class IV | 13 | (0.6%) | 7 | (0.4%) | 10 | (0.9%) | 8 | (0.9%) | |
| KCCQ Total | 70.0 ± 22.4 | | 67.4 ± 23.5 | | 64.8 ± 24.6 | | 62.2 ± 26.0 | | <0.001 |
| Symptom Score | | | | | | | | | |
| Heart Rate (bpm) | 70.6 ± 11.0 | | 71.8 ± 11.7 | | 72.4 ± 12.4 | | 71.8 ± 12.6 | | <0.001 |
| Systolic Blood | 128.4 ± 14.4 | | 129.7 ± 15.3 | | 129.2 ± 15.7 | | 131.4 ± 16.7 | | <0.001 |
| Pressure (mmHg) | | | | | | | | | |
| LVEF (%) | 51.9 ± 7.7 | | 52.7 ± 7.9 | | 52.9 ± 8.1 | | 53.1 ± 7.9 | | <0.001 |
| NT-proBNP | 822 [351, | | 1,500 [577, | | 2,076 [924, | | 2,716 [1224, | | <0.001 |
| (pg/mL) | 1873] ¹⁾ | | 2788] ¹⁾ | | 3743] ¹⁾ | | 5729] ¹⁾ | | |
| | 630 [302, | | 1,047 [478, | | 1,412 [682, | | 1,789 [849, | | |
| | 1279] ²⁾ | | 1826] ²⁾ | | 2439] ²⁾ | | 3551] ²⁾ | | |
| Prior HF | 1,170 | (57.5%) | 999 | (59.2%) | 723 | (61.7%) | 617 | (67.4%) | <0.001 |
| Hospitalization | | | | | | | | | |
| History of | 86 | (4.2%) | 74 | (4.4%) | 53 | (4.5%) | 49 | (5.4%) | 0.21 |
| LVEF <40% | | | | | | | | | |
| History of | 622 | (30.6%) | 641 | (38.0%) | 538 | (45.9%) | 554 | (60.5%) | <0.001 |
| Diabetes | | | | | | | | | |
| History of Atrial | 928 | (45.6%) | 960 | (56.9%) | 748 | (63.8%) | 553 | (60.4%) | <0.001 |
| Flutter/Fibrillation | | | | | | | | | |
| History of | 1,742 | (85.6%) | 1,500 | (88.9%) | 1,059 | (90.4%) | 847 | (92.6%) | <0.001 |
| Hypertension | | | | | | | | | |
| History of | 575 | (28.3%) | 408 | (24.2%) | 298 | (25.4%) | 213 | (23.3%) | 0.005 |
| Myocardial | | | | | | | | | |
| Infarction | | | | | | | | | |
| Loop diuretic | 1,997 | (98.1%) | 1,654 | (98.0%) | 1,158 | (98.8%) | 896 | (97.9%) | 0.75 |
| Beta blocker | 1,732 | (85.1%) | 1,437 | (85.1%) | 1,004 | (85.7%) | 758 | (82.8%) | 0.27 |
| ACEi | 839 | (41.2%) | 595 | (35.2%) | 380 | (32.4%) | 261 | (28.5%) | <0.001 |
| ARB | 820 | (40.3%) | 764 | (45.3%) | 557 | (47.5%) | 408 | (44.6%) | 0.002 |
| ARNI | 180 | (8.8%) | 138 | (8.2%) | 103 | (8.8%) | 75 | (8.2%) | 0.68 |
| SGLT2i | 203 | (10.0%) | 213 | (12.6%) | 185 | (15.8%) | 183 | (20.0%) | <0.001 |

Definition of ¹⁾ and ²⁾ may be found in Example 1b.

TABLE 14
KDIGO Risk Categories - U.S. HF Population in
NHANES - (Weighted n = 5,189,186 from 2015-2020)

| | | | UACR (mg/g) | | |
|---|---|---|---|---|---|
| | | | A1 | A2 | A3 |
| | | | <30 | 30-300 | >300 |
| Estimated | G1 | ≥90 | 6.4% | 2.4% | 0.5% |
| GFR | G2 | 60-89 | 28.6% | 11.1% | 2.9% |
| (mL/min/ | G3a | 45-59 | 15.5% | 8.1% | 2.6% |
| 1.73 m²) | G3b | 30-44 | 8.7% | 6.5% | 3.0% |
| | G4 | 15-29 | 1.6% | 1.2% | 0.9% |
| | G5 | <15 | 0.0% | 0.0% | 0.0% |

KDIGO Risk Categories - NHANES
Low (G1-A1, G2-A1): 47.5%
Moderate (G3-A1, G1-A2, G2-A3a): 32.0%
High (G3b-A1, G3a-A2, G2-A3, G1-A3): 11.6%
Very High: (G3a-A3, G3b-A2, G3b-A3, G4-A1, G4-A2, G4-A3, G5-A1, G5-A2, G5-A3): 8.8%

### Example 7—Characterization of Primary Aldosteronism Pathophysiology in Example 2

Primary aldosteronism (PA) is an underrecognized condition in patients with hypertension. PA, and its characteristic pathophysiology, is highly prevalent. PA pathophysiology has adverse consequences. The spectrum of PA pathophysiology is a targetable phenotype which Example 2 is well suited to identify. Identifying PA pathophysiology in Example 2: In HFpEF, where hypertension is a highly prevalent risk factor, it is hypothesized that chronic activation of the MR and resulting cardiac, vascular, and renal fibrosis may be driven by unrecognized PA in a meaningful proportion of patients. Thus, identifying underlying PA and its hallmark pathophysiology of renin-independent aldosterone production may help to a) ascertain a critical subgroup at elevated risk of adverse outcomes, b) identify those enriched for a clinical response to MR blockade with finerenone, and c) allow for evaluation of treatment effect based on the impact of finerenone on renin suppression, an emerging biomarker of adequate MR blockade for efficacy in PA.

AIM: Identify prevalence, prognostic significance, and treatment effect of PA pathophysiology at baseline.

### Baseline Biochemical Assessment:

a. Measurement of plasma renin activity (if available, based on sample collection protocols) and/or plasma renin concentration at baseline
b. Measurement of plasma aldosterone on the day of renin measurement at baseline
c. Measurement of serum potassium and urine potassium & sodium (if available) on the day of renin measurement at baseline
d. Analytical approach—in overall study population and in subset meeting criteria for apparent treatment resistant hypertension
   i. Designation based on baseline renin into suppressed vs. unsuppressed renin subgroups
   ii. Assessment of outcomes and treatment effect by baseline renin group
   iii. Assessment of outcomes and treatment effect by aldosterone, stratified by baseline renin group, and by aldosterone-to-renin ratio
   iv. Sensitivity analyses restricting population to:
      1. Patients on ACE/ARB (expected to raise renin, so suppression on these medications increases probability of PA pathophysiology)
      2. Patients not on Beta Blocker (expected to lower renin, so suppression on beta blockade is less specific for PA pathophysiology)

AIM: Evaluate impact of reversing PA pathophysiology by inducing a rise in renin with MR antagonist on outcomes and treatment effect.

### On-Treatment Biochemical Assessment:

e. Measurement of plasma renin activity (if available, based on sample collection protocols) and/or plasma renin concentration on treatment
f. Measurement of serum potassium and urine potassium & sodium (if available) on the day of renin measurement on treatment
g. Analytical approach
   i. Assessment of outcomes and treatment effect by change in renin
      1. Continuous relationship of change in renin to change in BP
      2. Assessment of outcomes and treatment effect comparing categorical change from suppressed to unsuppressed renin vs. persistently suppressed renin on treatment.

### Example 8—Cardio-Renal-Metabolic Overlap and Cardiorenal Risk in the Example 2

Patients with heart failure with mildly reduced or preserved ejection fraction (HFmrEF/HFpEF) commonly have cardio-renal-metabolic (CRM) comorbidities. The implications of these intersections on markers of risk and health status in this population are less well studied.

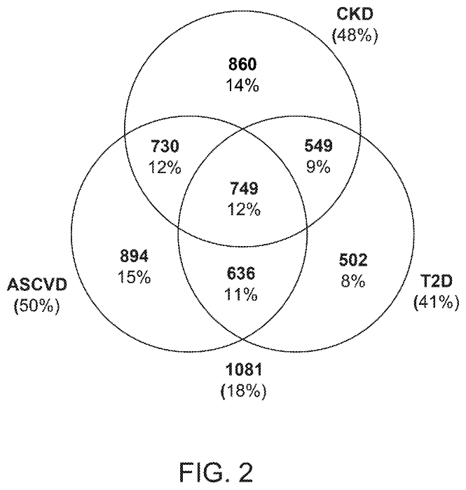

We first defined the clinical overlap in CRM conditions (atherosclerotic cardiovascular disease [ASCVD], type 2 diabetes [T2D], and chronic kidney disease [CKD; defined as eGFR<60 mL/min/1.73 m²). Distributions of markers of cardiorenal risk (UACR, NT-proBNP) and health status (KCCQ-total symptom score; range 0-100 with 100 reflecting best health status) across the CRM spectrum were examined. Among 6,001 participants in Example 2, 18%, 38%, 32%, and 12% had 0, 1, 2, or 3 CRM conditions (FIG. 2—Example 2 trial, participants with HFmref or HFpEF Cardo-Renal-Metabolic-Overlap). Median UACR was 12 [5-34] mg/g in those with 0 CRM conditions and 51 [14-248] mg/g in those with 3 CRM conditions (P<0.001). Median NT-proBNP was 1,352 [472-2,500] ¹⁾ pg/mL in those with 0 CRM conditions and 1,822 [654-3,983] ¹⁾ pg/mL in those with 3 CRM conditions (P<0.001). Mean KCCQ-TSS was 68.7±23.1 in those with 0 CRM conditions and 63.8+25.5 in those with 3 CRM conditions (P<0.001); see Table 15-Markers of Cardiorenal Risk and Heath Status by Presence of Cardio-Renal-Metabolic Conditions). Four out of five patients with HFmrEF/HFpEF have at least 1 coexisting CRM condition. Patients with greater CRM overlap have higher markers of cardiorenal progression and worse health status and symptom burden. These data support prioritizing treatment optimization and care approaches in patients with HF and high CRM overlap.

TABLE 15
Markers of Cardiorenal Risk and Health Status by Presence of Cardio-Renal-Metabolic Conditions
# of Cardio-Renal-Metabolic Conditions

| | 0 | | 1 | | | | | | |
|---|---|---|---|---|---|---|---|---|---|
| | No | | DM, | CKD, | | 2 | DM, | 3 | |
| | ASCVD, | ASCVD, | no | no | ASCVD, | ASCVD, | CKD, | ASCVD, | |
| | no DM, | no DM, | ASCVD, | ASCVD, | DM, | CKD, | no | DM, | |
| | no CKD | no CKD | no CKD | no DM | no CKD | no DM | ASCVD | CKD | |
| | n = 1,081 | n = 894 | n = 502 | n = 860 | n = 636 | n = 730 | n = 549 | n = 749 | |
| UACR | 12 | 11 | 20 | 18 | 23 | 18 | 42 | 51 | p < 0.001 |
| (mg/g) | [5, 34] | [5, 30] | [8, 87] | [8, 56] | [7, 75] | [6, 52] | [13, 190] | [14, 248] | |
| NT- | 1352 | 987 | 1127 | 2110 | 1008 | 2004 | 2114 | 1822 | p < 0.001 |
| proBNP | [472, | [390, | [365, | [1017, | [377, | [750, | [898, | [654, | |
| (pg/mL) | 2500] ¹⁾ | 2093] ¹⁾ | 2279] ¹⁾ | 3844] ¹⁾ | 2034] ¹⁾ | 4119] ¹⁾ | 3741] ¹⁾ | 3983] ¹⁾ | |
| KCCQ | 68.7 ± | 71.2 ± | 66.0 ± | 65.3 ± | 68.5 ± | 67.8 ± | 62.3 ± | 63.8 ± | p < 0.001 |
| TSS | 23.1 | 22.5 | 24.5 | 23.8 | 23.7 | 22.7 | 25.4 | 25.5 | |

Definition of ¹⁾ and ²⁾ in may be found in Example 1b.

### Example 9—An Integrated Pooled Analysis of Finerenone in >19,000 Participants across 3 Phase III Trials of HF & CKD (FINE-HEART)

Background: The non-steroidal mineralocorticoid receptor antagonist (MRA), finerenone, has been independently studied in prospective randomized trials of patients with type 2 diabetes and chronic kidney disease (CKD) and separately in patients with heart failure (HF) with mildly reduced or preserved ejection fraction. However, none of these trials was individually powered to evaluated treatment effects on cardiovascular mortality or efficacy in key subgroups subpopulations such as those with overlapping cardio-kidney-metabolic (CKM) intersection. In light of the strong epidemiological overlap and shared mechanistic drivers of the enrolled populations, a pooled assessment summarizing the totality of evidence from these cardio-kidney trials was prespecified.

Methods: We conducted a protocol prespecified participant-level pooled analysis of 3 phase III global, multicenter, double-blind, placebo-controlled randomized clinical trials of Finerenone (FINE-Heart). The FIDELITY program (encompassing the FIDELIO-DKD and FIGARO-DKD trials) enrolled participants with type 2 diabetes and CKD with albuminuria (UACR≥30 mg/g) on maximally tolerated doses of renin-angiotensin system inhibitors across 48 countries. FINEARTS-HF enrolled participants with symptomatic HF, LVEF ≥40%, elevated natriuretic peptides, and evidence of structural heart disease across 37 countries. Baseline characteristics were summarized across the pooled population including the prevalence of key CKM comorbidities (HF, diabetes, and CKD eGFR<60 mL/min/1.73 m² or UACR≥30 mg/g]) and their overlap. The primary endpoint for FINE-HEART was cardiovascular death, centrally adjudicated by independent clinical endpoints committees in each of the trials. Primary analyses of FINE-HEART have been predefined in a dedicated Statistical Analysis Plan.

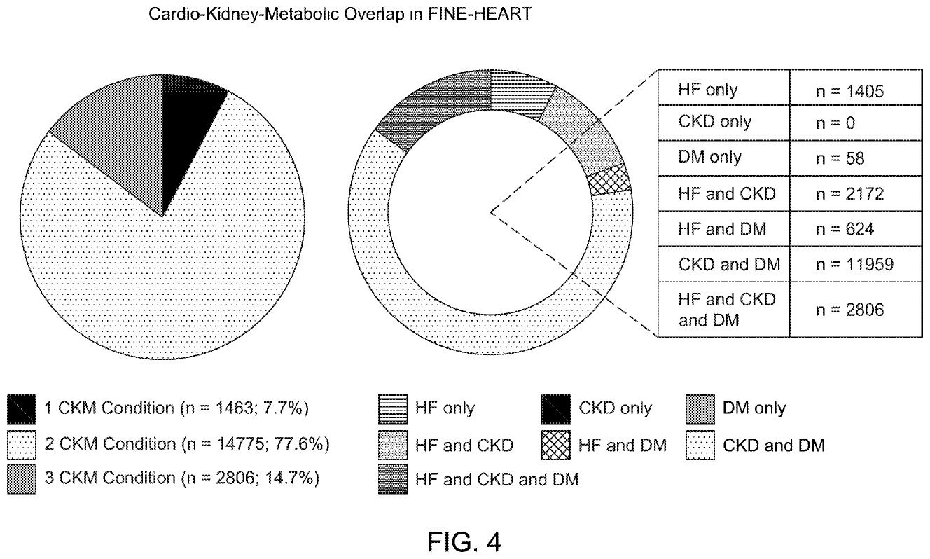

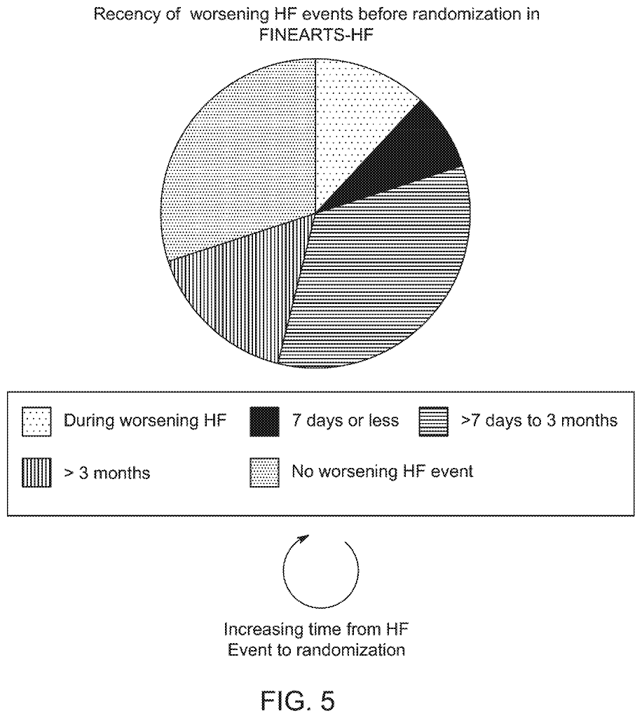

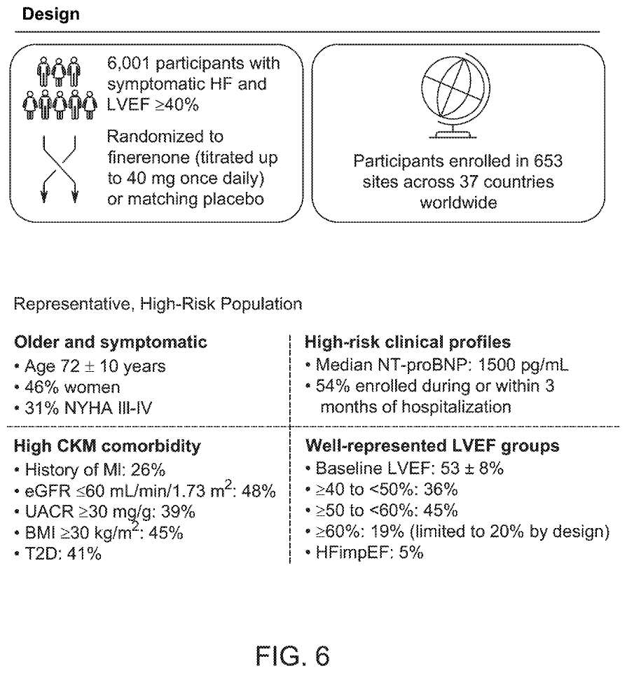

Results: Overall, FINE-HEART comprised 19,027 participants from these three trials (mean age 67±10 years; 35% women). See Table 16. Participants were at high kidney risk for CKD progression with either reduced eGFR (30% with eGFR <45 and 26% with eGFR 45 to 60 mL/min/1.73 m²) and/or albuminuria (31% with “A2” UACR 30-300 mg/g and 49% with “A3” UACR>300 mg/g). Prevalence of CKM comorbidities was high with history of HF (37%), diabetes (81%), and CKD in 89% resulting in 8% with1 CKM condition, 78% having 2 CKM conditions, and 15% with all 3 CKM conditions (HF, diabetes, and CKD). See FIG. 4. FIG. 4 shows the Cardio-Kidney-Metabolic Overlap in FINE-HEART. Overall, 1,691 (9%) of participants were co-treated with an SGLT2i. Over median follow-up of 3 years in the FIDELITY program, there were 686 cardiovascular deaths (1.7 per 100py). While follow-up is still underway in the event-driven FINEARTS-HF trial, under the assumptions of sample size determination, approximately 535 cardiovascular deaths are expected, resulting in a total of 1,221 expected cardiovascular deaths in FINE-HEART.

Conclusions: FINE-HEART will have increased precision to robustly assess the safety and efficacy of the non-steroidal MRA finerenone on cardiovascular death and other cardio-kidney outcomes, and will be enriched for participants with high burden of CKM multimorbidity.

TABLE 16
Baseline Characteristics of FINE-HEART (n = 19,027)

| | |
|---|---|
| Age | 67 ± 10 |
| Women | 35% |
| BMG (kg/m²) | 31 ± 6 |
| Systolic blood pressure (BP) (mmHg) | 134 ± 15 |
| eGFR (mL/min/1.73 m²) | 59 ± 21 |
| Below 25 | 1% |
| 25 to below 45 | 29% |
| 45 to below 60 | 26% |
| Equal to or above 60 | 44% |
| UACR (mg/g) | 640 ± 913 |
| A1: below 30 | 20% |
| A2: 30 to below 300 | 31% |
| A3: equal to or above 300 | 49% |
| Hemoglobin A1c (%) | 7.3 ± 1.4 |
| History of HF | 37% |
| History of Diabetes | 81% |
| History of AF/AFL | 25% |
| Diuretic | 63% |
| ACEi | 38% |
| ARB | 56% |
| SGLT2i | 9% |

### Example 10—Generalizability of the FINEARTS-HF Trial to the U.S. Population Across the Spectrum of Kidney Risk

Example 2 evaluated the efficacy and safety of finerenone in patients with heart failure (HF) and left ventricular ejection fraction ≥40%, with or without diabetes, and across a broad range of kidney function (FIG. 1). We examined the distribution of kidney risk represented in the Example 2 trial and compared it with that in the US HF population.

Serial cross-sectional analyses of adults from the National Health and Nutrition Examination Surveys (NHANES; 2015-2016, 2017-2018, and 2019-2020) identified a US sample. Samples were weighted to derive a nationally representative cohort of adults with HF (aged ≥40 years with HF, estimated glomerular filtration rate (eGFR) ≥25 mL/min/1.73 m², and potassium≤5.0 mmol/L). Kidney risk was analyzed per Kidney Disease Improving Global Outcomes (KDIGO) risk categories, defined by eGFR and urine albumin-to-creatinine ratio (UACR).

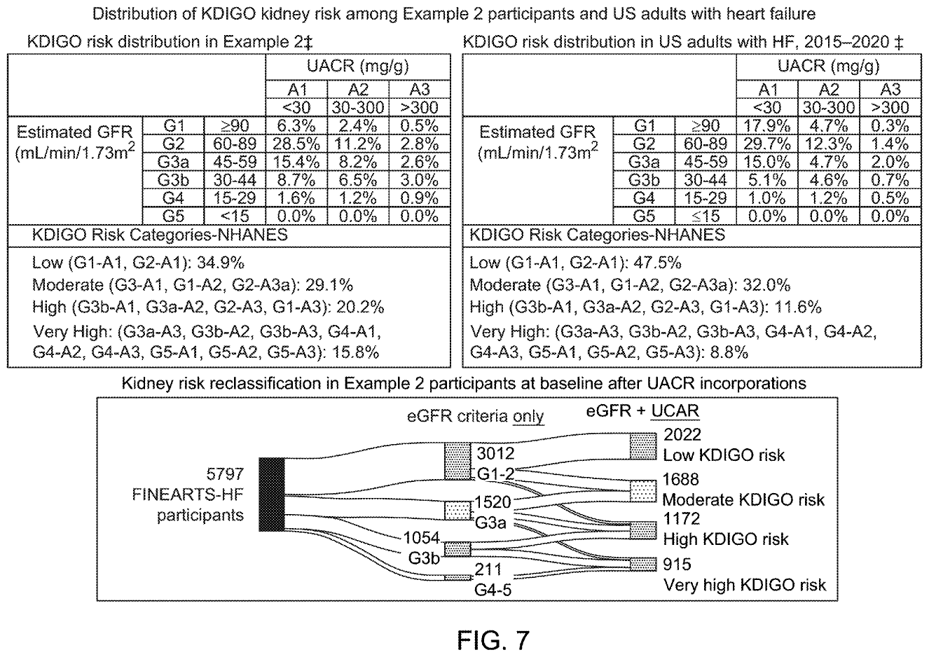

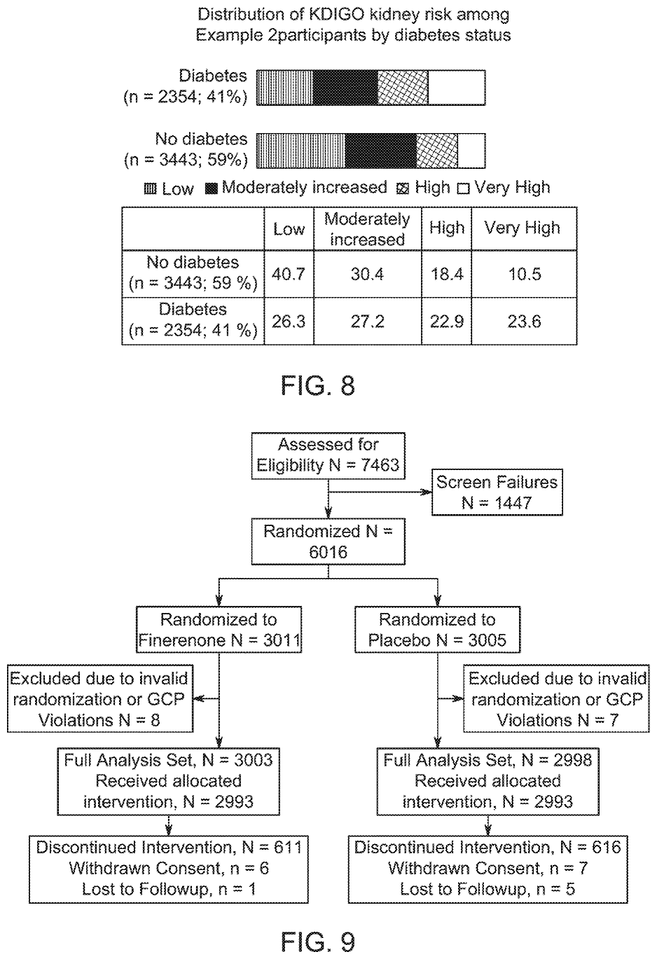

Among 6,001 randomized participants, 5,797 (97%) could be assigned to a KDIGO category. Median baseline eGFR (mL/min/1.73 m² [interquartile range {IQR}]) was 61 (47-77); 2,785 (48%) had an eGFR <60 mL/min/1.73 m². Median baseline UACR (mg/g [IQR]) was 18 (7-67); 2,279 (39%) had a UACR ≥30 mg/g. In Example 2, 34.6%, 29.1%, 20.2%, and 15.8% of participants were classified as KDIGO low, moderate, high, and very high risk, respectively. Using NHANES data, we estimated that of 5,189,186 US individuals with HF (95% confidence interval 4,333,114 to 6,045,257), 47.6%, 32.0%, 11.6%, and 8.8% would be classified as KDIGO low, moderate, high, and very high risk, respectively (FIG. 7; †Graphical display of the distributions of KDIGO kidney risk among Example 2 participants with available eGFR and UACR data (n=5,797) at baseline. $Graphical display of the distributions of KDIGO kidney risk among weighted NHANES (2015 to March 2020) sample of US adults (weighted n =5,189,186) with HF, age ≥40 years, eGFR ≥25 mL/min/1.73 m², and serum potassium ≥5 mmol/L. § Sankey diagram showing reclassification of KDIGO kidney risk after incorporation of eGFR and UACR, compared with eGFR stages alone. eGFR, estimated glomerular filtration rate; HF, heart failure; KDIGO, Kidney Disease Improving Global Outcomes; NHANES, National Health and Nutrition Examination Surveys; UACR, urine albumin-to-creatinine ratio); p<0.001 for comparison with KDIGO risk distribution in Example 2. In Example 2 participants without diabetes, 40.7%, 30.4%, 18.4%, and 10.5% were classified as KDIGO low, moderate, high, and very high risk, respectively (FIG. 8). In Example 2 participants with diabetes, 26.3%, 27.2%, 22.9%, and 23.6% were classified as KDIGO low, moderate, high, and very high risk, respectively (FIG. 8).

Incorporating UACR reclassified kidney risk in approximately one in three Example 2 participants and U.S. adults with HF. This emphasizes the importance of albuminuria as part of a comprehensive risk assessment in the HF population.

### Example 11—Tablets comprising finerenone (4S)-4-(4-cyano-2-methoxyphenyl)-5-ethoxy-2,8-dimethyl-1,4-dihydro-1,6-naphthyridine-3-carboxamide of the formula (I)

A granulate solution of the compound of formula (I) in crystalline form in micronized form, hypromellose 5 cP, sodium lauryl sulfate in purified water was prepared. Microcrystalline cellulose, lactose monohydrate and croscarmellose sodium were mixed in a container or a fluidized bed granulator (premix). The premix and the granulate solution were granulated in the fluid-bed granulator. The lubricant magnesium stearate was added after the granules were dried and sieved. A ready-to-press mixture was thus produced. The ready-to-press mixture was pressed into tablets using a rotary tablet press. A homogeneous coating suspension was made from hypromellose, talc, titanium dioxide, yellow iron oxide, red iron oxide and purified water. The coating suspension was sprayed onto the tablets in a suitable coating device.

TABLE 17
Tablets (number 1 to 7) obtained by the process described above

| | | Tablets | | | | | | |
|---|---|---|---|---|---|---|---|---|
| | | 1 | 2 | 3 | 4 | 5 | 6 | 7 |
| Composition | | [mg] | [mg] | [mg] | [mg] | [mg] | [mg] | [mg] |
| | Finerenone, | 1.25 | 2.50 | 5.00 | 7.50 | 10.00 | 15.00 | 20.00 |
| | micronized | | | | | | | |
| Excipients | Cellulose | 73.80 | 72.50 | 69.90 | 67.30 | 64.70 | 62.00 | 59.30 |
| | microcrystalline | | | | | | | |
| | Crosscarmellose | 4.50 | 4.50 | 4.50 | 4.50 | 4.50 | 4.50 | 4.50 |
| | sodium | | | | | | | |
| | Hypromellose 5 | 4.50 | 4.50 | 4.50 | 4.50 | 4.50 | 4.50 | 4.50 |
| | cP | | | | | | | |
| | Lactose | 45.00 | 45.00 | 45.00 | 45.00 | 45.00 | 42.50 | 40.00 |
| | monohydrate | | | | | | | |
| | Magnesium | 0.90 | 0.90 | 0.90 | 0.90 | 0.90 | 0.90 | 0.90 |
| | stearate | | | | | | | |
| | Sodium | 0.05 | 0.10 | 0.20 | 0.30 | 0.40 | 0.60 | 0.80 |
| | laurylsulfate | | | | | | | |
| | Weight | 130.00 | 130.00 | 130.00 | 130.00 | 130.00 | 130.00 | 130.00 |
| | (uncoated | | | | | | | |
| | tablet) | | | | | | | |
| Film- | Hypromellose 5 | 3.0336 | 3.0336 | 3.0336 | 3.0336 | 3.0336 | 3.0336 | 3.0336 |
| coating | cP | | | | | | | |
| | Titanium | 2.3196 | 2.3196 | 2.3196 | 2.3196 | 2.3196 | 2.3196 | 2.3196 |
| | dioxide | | | | | | | |
| | Talcum | 0.6072 | 0.6072 | 0.6072 | 0.6072 | 0.6072 | 0.6072 | 0.6072 |
| | Iron oxide | 0.0324 | 0.0324 | 0.0324 | 0.0324 | 0.0324 | 0.0324 | 0.0324 |
| | yellow | | | | | | | |
| | Iron oxide | 0.0072 | 0.0072 | 0.0072 | 0.0072 | 0.0072 | 0.0072 | 0.0072 |
| | red | | | | | | | |
| | Weight | 6.0000 | 6.0000 | 6.0000 | 6.0000 | 6.0000 | 6.0000 | 6.0000 |
| | (film-coating) | | | | | | | |
| | Weight | 136.00 | 136.00 | 136.00 | 136.00 | 136.00 | 136.00 | 136.00 |
| | (coated tablet) | | | | | | | |

### Example 12—Tablets comprising finerenone (4S)-4-(4-cyano-2-methoxyphenyl)-5-ethoxy-2,8-dimethyl-1,4-dihydro-1,6-naphthyridine-3-carboxamide of the formula (I)

A granulate suspension of the compound of formula (I) in crystalline form in micronized form, hypromellose, sodium lauryl sulfate in purified water was prepared. Microcrystalline cellulose, lactose monohydrate and croscarmellose sodium were mixed in a container or a fluidized bed granulator (premix). The premix and the granulate solution were granulated in the fluid-bed granulator. The lubricant magnesium stearate was added after the granules were dried and sieved. A ready-to-press mixture was thus produced. The ready-to-press mixture was compressed into tablets using a rotary tablet press. A homogeneous coating suspension was made from hypromellose, talc, titanium dioxide, yellow iron oxide, red iron oxide and/or black iron oxide and purified water. The coating suspension was sprayed onto the tablets in a suitable coating device. The composition of the tablets obtained by the process described are listed in table 18.

TABLE 18
Tablets (8 to 12 obtained) obtained by the process described above

| | | Tablets | | | | |
|---|---|---|---|---|---|---|
| | | 8 | 9 | 10 | 11 | 12 |
| Composition | | [mg] | [mg] | [mg] | [mg] | [mg] |
| | Finerenone | 40 | 5 | 10 | 20 | 40 |
| | micronized | | | | | |
| Excipients | Cellulose | 110 | 69.9 | 64.7 | 59.3 | 110 |
| | microcrystalline | | | | | |
| | Croscarmellose | 15 | 4.5 | 4.5 | 4.5 | 15 |
| | sodium | | | | | |
| | Hypromellose 5 cP | 7 | 4.5 | 4.5 | 4.5 | 7 |
| | Lactose monohydrate | 25 | 45 | 45 | 40 | 25 |
| | Magnesium stearate | 1.4 | 0.9 | 0.9 | 0.9 | 1.4 |
| | Sodium laurilsulfate | 1.6 | 0.2 | 0.4 | 0.8 | 1.6 |
| | Purified water | q.s. | q.s. | q.s. | q.s. | q.s. |
| | in bulk | | | | | |
| | Weight | 200 | 130 | 130 | 130 | 200 |
| | (uncoated tablet) | | | | | |
| Film | Hypromellose 5 cP | 3.5392 | 3 | 3 | 3 | 3.5 |
| coating | Talc | 0.7084 | 0.6 | 0.6 | 0.6 | 0.7 |
| | Titanium dioxide | 2.7062 | 2.36 | 2.28 | 1.92 | 2.222 |
| | Ferric oxide yellow | 0.0378 | — | — | 0.48 | 0.473 |
| | Ferric oxide red | 0.0084 | — | 0.12 | — | 0.105 |
| | Ferric oxide black | — | 0.04 | — | — | — |
| | Purified water | q.s. | q.s. | q.s. | q.s. | q.s. |
| | in bulk | | | | | |
| | Weight (film | 7 | 6 | 6 | 6 | 7 |
| | coating) | | | | | |

| | | | | | |
|---|---|---|---|---|---|
| Weight | 207 | 136 | 136 | 136 | 207 |
| (coated tablet) | | | | | |

As film coatings, commercially available film coatings can be used for the tablets disclosed in tables 17 and 18 above. Examples are Opadry® film coatings such as Opadry® 02A275000 light gray, Opadry® 02A240005 light pink or Opadry® 02A220009 light yellow.

### Example 13—Finerenone in Heart Failure with Mildly Reduced or Preserved Ejection Fraction

Background: Steroidal mineralocorticoid receptor antagonists reduce morbidity and mortality in patients with heart failure with reduced ejection fraction but their efficacy in those with heart failure with mildly reduced or preserved ejection has not been established. We tested the efficacy and safety of the non-steroidal MRA finerenone in patients with heart failure and mildly reduced or preserved ejection fraction.
Methods: 6,001 patients with heart failure and a left ventricular ejection fraction ≥40% were randomly assigned to finerenone (up to 40 mg once daily) or matching placebo. The primary outcome was a composite of total worsening heart failure events and cardiovascular death.
Results: Over a median of 32 months, there were 1,083 primary events in 624 patients in the finerenone group and 1,283 primary events in 719 patients in the placebo group (rate ratio, 0.84; 95% confidence interval [CI], 0.74 to 0.95; P=0.007). There were 842 total worsening heart failure events in the finerenone group and 1,024 total worsening heart failure events in the placebo group (rate ratio, 0.82; 95% confidence interval [CI], 0.71 to 0.94, p=0.006). The incidence of death from cardiovascular causes was 8.1% and 8.7%, respectively (hazard ratio, 0.93; 95% CI, 0.78 to 1.11). Finerenone increased the risk of hyperkalemia and reduced the risk of hypokalemia.
Conclusions: Finerenone reduced the composite of total worsening heart failure events and cardiovascular death in patients with heart failure and mildly reduced or preserved ejection fraction. (NCT04435626).

### In Detail:

### Methods

### Detailed Inclusion and Exclusion Criteria

Study Population: Patients with a diagnosis of HF, NYHA class II-IV, and documented LVEF of ≥40%.

### Inclusion Criteria

Participants are eligible to be included in the study only if all of the following criteria apply:

### Age

Participant must be aged 40 years and older, at the time of signing the informed consent.

### Type of Participant and Disease Characteristics:

Diagnosis of heart failure with NYHA class II-IV, ambulatory or hospitalized primarily for heart failure (if a hospitalized patient cannot be randomized as an in-patient, randomization as soon as possible after discharge is encouraged)
On diuretic treatment for at least 30 days prior to randomization
Documented LVEF of ≥40% measured by any modality within the last 12 months, at the latest at screening; if several values are available, the most recent one shall be reported. If LVEF was not measured in the past 12 months, a new measurement may be done at screening
Structural heart abnormalities based on any local imaging measurement within the last 12 months, latest at screening, defined by at least 1 of the following findings:
   LAD ≥3.8 cm, LAA ≥20 cm², LAVI >30 mL/m², LVMI ≥115 g/m² (♂)/95 g/m² (♀), septal thickness or posterior wall thickness ≥1.1 cm
NT-proBNP ≥300 μg/mL (BNP ≥100 μg/mL) in sinus rhythm and patient does not have an ongoing diagnosis of paroxysmal atrial fibrillation or NT-proBNP ≥900 pg/mL (BNP ≥300 pg/mL) in atrial fibrillation (or if atrial fibrillation status is unknown or if patient has an ongoing diagnosis of paroxysmal atrial fibrillation) for participants obtained at the following time:
Within 90 days prior to randomization if patient had been hospitalized for HF requiring initiation or change in HF therapy or if patient had an urgent visit for HF requiring intravenous (IV) diuretic therapy, both within 90 days prior to randomization
OR
Within 30 days prior to randomization if patient has not been hospitalized for HF nor had an urgent HF visit within the past 90 days.

### Sex

Male or female. Women of childbearing potential can only be included in the study if a pregnancy test is negative at screening and baseline and if they agree to use adequate contraception which is consistent with local regulations regarding the methods for contraception for those participating in clinical trials.

### Informed Consent

Capable of giving signed informed consent which includes compliance with the requirements and restrictions listed in the informed consent form (ICF) and in this protocol.

### Exclusion Criteria

Participants are excluded from the study if any of the following criteria apply:

### Medical Conditions

eGFR <25 mL/min/1.73 m² at either screening or randomization visit.
NOTE: one reassessment of eGFR is allowed at the screening and randomization visit, respectively
Serum/plasma potassium >5.0 mmol/L at either screening or randomization visit. NOTE: one reassessment of potassium is allowed at the screening and randomization visit, respectively
Acute inflammatory heart disease, e.g. acute myocarditis, within 90 days prior to randomization
Myocardial infarction or any event which could have reduced the ejection fraction within 90 days prior to randomization
Coronary artery bypass graft surgery in the 90 days prior to randomization
Percutaneous coronary intervention in the 30 days prior to randomization
Stroke or transient ischemic cerebral attack within 90 days prior to randomization
Probable alternative cause of participants' HF symptoms that in the opinion of the investigator primarily accounts for patient's dyspnea such as significant pulmonary disease, anemia or obesity. Specifically, patients with the below are excluded:
   Severe pulmonary disease requiring home oxygen, or chronic oral steroid therapy
   History of primary pulmonary arterial hypertension
   Hemoglobin <10 g/dL
   Valvular heart disease considered by the investigator to be clinically significant
   Body mass index (BMI)>50 kg/m² at screening.
Systolic blood pressure (SBP) ≥160 mmHg if not on treatment with ≥3 blood pressure lowering medications or ≥180 mmHg irrespective of treatments on 2 consecutive measurements at least 2-minute apart, at screening or at randomization
Life-threatening or uncontrolled arrhythmias at screening and/or randomization including but not limited to sustained ventricular tachycardia and atrial fibrillation, or atrial flutter with resting ventricular rate >110 bpm
Symptomatic hypotension with mean systolic blood pressure <90 mmHg at screening or at randomization
Any primary cause of HF scheduled for surgery, e.g. valve disease such as severe aortic stenosis or severe mitral regurgitation by the time of screening or randomization
History of peripartum cardiomyopathy, chemotherapy induced cardiomyopathy, viral myocarditis, right heart failure in absence of left-sided structural disease, pericardial constriction, genetic hypertrophic cardiomyopathy, or infiltrative cardiomyopathy including amyloidosis
Presence of left ventricular assist device by the time of screening or randomization
History of hyperkalemia or acute renal failure during MRA treatment for
>7 consecutive days, leading to permanent discontinuation of the MRA treatment
Pregnant or nursing (lactating) women, where pregnancy is defined as the state of a female after conception and until the termination of gestation, confirmed by a positive human chorionic gonadotrophin urine or serum test
Known hypersensitivity to the study intervention (active substance or excipients)
Hepatic insufficiency classified as Child-Pugh C at screening or randomization
Addison's disease.

### Prior/Concomitant Therapy

Requirement of any IV vasodilating drug (e.g., nitrates, nitroprusside), any IV natriuretic peptide (e.g., nesiritide, carperitide), any IV positive inotropic agents, or mechanical support (intra-aortic balloon pump, endotracheal intubation, mechanical ventilation, or any ventricular assist device) within 24 hours prior to randomization
Participants who require treatment with more than one ACEI, ARB or angiotensin-receptor neprilysin inhibitor (ARNI), or two simultaneously at randomization
Continuous (at least 90 days) treatment with an MRA (e.g., spironolactone, eplerenone, canrenone, esaxerenone) within 12 months prior to screening. Last intake at least 30 days before randomization. Treatment with MRA should not be interrupted with the purpose of enrollment into the study
Concomitant treatment with any renin inhibitor or potassium-sparing diuretic that cannot be stopped prior to randomization and for the duration of the treatment period
Concomitant systemic therapy with potent cytochrome P450 isoenzyme 3A4 (CYP3A4) inhibitors (e.g., itraconazole, ritonavir, indinavir, cobicistat, clarithromycin) or moderate or potent CYP3A4 inducers, that cannot be discontinued 7 days prior to randomization and for the duration of the treatment period.

### Other Exclusions

Any other condition or therapy, which would make the participant unsuitable for this study and will not allow participation for the full planned study period (e.g., active malignancy or other condition limiting life expectancy to less than 12 months)
Previous assignment to treatment during this study
Participation in another interventional clinical study (e.g., Phase 1 to 3 clinical studies) or treatment with another investigational medicinal product within 30 days prior to randomization
Close affiliation with the investigational site; e.g., a close relative of the investigator, dependent person (e.g., employee or student of the investigational site)
Known current alcohol and/or illicit drug abuse that may interfere with the participant's safety and/or compliance at the discretion of the investigator
Participant is in custody by order of an authority or a court of law.

### Trial Design and Oversight

FINEARTS-HF was an international, multicenter, parallel-group, event-driven, randomized, double-blind trial in patients with chronic heart failure and left ventricular ejection fraction of 40% or greater, comparing the effect of finerenone, titrated to 20 mg (if baseline eGFR≤60 mL/min/1.73 m²) or 40 mg once daily (if baseline eGFR>60 mL/min/1.73 m²) versus placebo, in addition to usual therapy. Details regarding the design of the trial have been, e.g., above.

### Trial Participants

Eligibility requirements included stabilized heart failure in either inpatients or outpatients who were at least 40 years of age, had a left ventricular ejection fraction of 40% or more (including those with prior left ventricular ejection fraction less than 40%), evidence of structural heart disease and elevation of natriuretic peptides. Detailed inclusion and exclusion criteria have been described above. The patient flow is shown in FIG. 9.

### Trial Procedures and Outcomes

All patients provided written informed consent. Patients who met the inclusion and exclusion criteria were randomly assigned to receive finerenone (at a starting dose of 10 mg titrated to 20 mg once daily in patients with a baseline eGFR £ 60 ml/min/1.73 m², or a starting dose of 20 mg titrated to 40 mg once daily in those with a baseline eGFR >60 ml/min/1.73 m²) or matching placebo. The primary outcome was a composite of cardiovascular death and total worsening heart failure events (defined as either an unplanned hospitalization for heart failure or an urgent heart failure visit). Secondary objectives were to determine whether finerenone was superior to placebo in reducing the total number of worsening heart failure events; in the Kansas City Cardiomyopathy Questionnaire (KCCQ) total symptom score (TSS) at Months 6, 9, 12 (scores range from 0 to 100, with higher scores indicating fewer symptoms and physical limitations) and improving NYHA class at month 12; in reducing a composite kidney outcome (defined as a sustained 50% or greater decline in eGFR, a sustained decline in eGFR to <15 ml/min/1.73 m² or initiation of chronic dialysis or kidney transplantation); and in reducing all-cause mortality. All primary events were adjudicated by an independent clinical events committee blinded to treatment assignment based on prespecified criteria. Adverse events and serious adverse events were collected and reported as both treatment-emergent (occurring in patients who received at least one dose of study drug and within 3 days of permanent discontinuation) and throughout the study.

### Statistical Analysis

The primary analysis compared the total rate of cardiovascular death and worsening heart failure events (either heart failure hospitalization or urgent heart failure visits) in an intention-to-treat approach using the semiparametric proportional rates method of Lin et al. (Lin D Y, Wei L J, Yang I, Ying Z. Semiparametric regression for the mean and rate functions of recurrent events. J R Stat Soc Series B Stat Methodol 2000; 62:711-730. https://doi.org/10.1111/1467-9868.00259) stratified according to geographic region and baseline LVEF (<60%, ≥60%). Significance for the primary outcome was set at two-sided p<0.0497 based on an adjustment for the interim analysis.

It was determined that 2,375 total (first and recurrent) primary composite events would be required to provide 90% power to detect an overall 19% lower event rate in the finerenone group. We estimated that the target number of events would be obtained by enrolling approximately 5,500 subjects over 24 months with a minimum follow-up of 18 months. Due to blinded event rates being lower than those assumed in the sample size calculation, the planned number of randomized subjects was increased to approximately 6,000 in July 2022. The target number of primary composite events was not changed.

The secondary hypotheses were tested hierarchically as follows based on the rejection of the primary null hypothesis: total HF events; KCCQ total symptom score improvement and NYHA class improvement (tested simultaneously using the Bonferroni-Holm procedure); and the composite kidney endpoint. All-cause mortality was tested outside this hierarchy, if the primary null hypothesis was rejected, at a nominal two-sided significance level of 5%.

One non-binding interim analysis for futility was performed when approximately 30% of the required total number of primary endpoint events were observed, and one formal interim analysis for efficacy was performed when approximately two-thirds of the required total number of primary endpoint events were observed. Following both pre-specified interim analyses, the Data Monitoring Committee recommended continuing the trial unchanged.

### Results

### Enrollment, Randomization, and Follow-up

Between 14 Sep. 2020 and 10 Jan. 2023, 7,463 patients from 654 sites across 37 countries were screened. Overall, 6,016 patients were randomized to receive finerenone or placebo. One patient was randomized twice by mistake, and both entries were excluded, and 13 patients from a single site were excluded from the primary analysis due to significant Good Clinical Practice violations. A total of 6,001 patients were therefore validly randomized and included in the primary analysis. At the end of the trial (Jun. 14, 2024), 13 patients had withdrawn consent and 6 were otherwise lost to follow-up. The median duration of follow-up was 32 months in each group.

The characteristics of the patients at baseline were balanced between the two treatment groups. See Table 19.

TABLE 19
Baseline Characteristics of Randomized Participants

| | Finerenone | | Placebo | |
|---|---|---|---|---|
| | (N = 3,003) | | (N = 2,998) | |
| Age | 71.9 ± 9.6 | | 72.0 ± 9.7 | |
| Women | 1,355 | (45.1%) | 1,377 | (45.9%) |
| Race | | | | |
| Asian | 497 | (16.6%) | 499 | (16.6%) |
| Black | 49 | (1.6%) | 39 | (1.3%) |
| Other | 91 | (3.0%) | 91 | (3.0%) |
| White | 2,366 | (78.8%) | 2,369 | (79.0%) |
| Region | | | | |
| Asia | 493 | (16.4%) | 490 | (16.3%) |
| Eastern Europe | 1,329 | (44.3%) | 1,321 | (44.1%) |
| Latin America | 322 | (10.7%) | 319 | (10.6%) |
| North America | 235 | (7.8%) | 236 | (7.9%) |
| Western Europe, Oceania | 624 | (20.8%) | 632 | (21.1%) |
| and Others | | | | |
| Any prior HF Hospitalization | 1,797 | (59.8%) | 1,822 | (60.8%) |
| Recency of Heart Failure | | | | |
| Event | | | | |
| <=7 days from randomization | 609 | (20.3%) | 610 | (20.3%) |
| >7 days-<=3 months | 1,030 | (34.3%) | 998 | (33.3%) |
| >3 months or no index HF | 1,364 | (45.4%) | 1,390 | (46.4%) |
| event | | | | |
| Systolic Blood | 129.5 ± 15.3 | | 129.3 ± 15.3 | |
| Pressure (mmHg) | | | | |
| Body Mass Index (kg/m2) | 29.9 ± 6.1 | | 30.0 ± 6.1 | |
| Creatinine (mg/dL) | 1.1 ± 0.3 | | 1.1 ± 0.4 | |
| eGFR (mL/min/1.73 m2) | 61.9 ± 19.4 | | 62.3 ± 20.0 | |
| eGFR <60 mL/min/1.73 m² | 1,451 | (48.3%) | 1,437 | (47.9%) |
| UACR (mg/g) | 18 | [7, 67] | 19 | [7, 66] |
| potassium (mmol/L) | 4.4 ± 0.5 | | 4.4 ± 0.5 | |
| LVEF mean (%) | 52.6 ± 7.8 | | 52.5 ± 7.8 | |
| LVEF <50% | 1,093 | (36%) | 1,079 | (36%) |
| LVEF ≥50% and <60% | 1,329 | (44%) | 1,345 | (49%) |
| LVEF ≥60% | 575 | (19%) | 572 | (19%) |
| NT-pro BNP (pg/mL) | 1,052 | [467, 1937] | 1,028 | [433, 1963] |
| (median, IQR) | | | | |
| NYHA class | | | | |
| Missing | 1 | (<0.1%) | 0 | (0.0%) |
| NYHA Class II | 2,081 | (69.3%) | 2,065 | (68.9%) |
| NYHA Class III | 903 | (30.1%) | 910 | (30.4%) |
| NYHA Class IV | 18 | (0.6%) | 23 | (0.8%) |
| Medical History | | | | |
| Hypertension | 2,640 | (87.9%) | 2,685 | (89.6%) |
| Diabetes Mellitus | 1,217 | (40.5%) | 1,222 | (40.8%) |
| Atrial Fibrillation on | 1,165 | (38.8%) | 1,128 | (37.6%) |
| ECG at baseline | | | | |
| Stroke | 355 | (11.8%) | 353 | (11.8%) |
| Myocardial Infarction | 784 | (26.1%) | 757 | (25.3%) |
| Medication Use | | | | |
| Beta-blocker | 2,541 | (84.6%) | 2,554 | (85.2%) |
| ACEI | 1,083 | (36.1%) | 1,072 | (35.8%) |
| ARB (w/o ARNI) | 1,047 | (34.9%) | 1,055 | (35.2%) |
| ARNI | 256 | (8.5%) | 257 | (8.6%) |
| Calcium channel blocker | 958 | (31.9%) | 1,010 | (33.7%) |
| SGLT-2 inhib. | 393 | (13.1%) | 424 | (14.1%) |
| Loop diuretic | 2,618 | (87.2%) | 2,621 | (87.4%) |

Patients had a mean LVEF of 53±8%; 36% had LVEF <50%, 45% had LVEF between 50% and 59%, and 19% had LVEF ≥60%. The majority were New York Heart Association functional class II (69%), and 1,219 (20%) patients were enrolled during or within 7 days of a worsening HF event. Baseline medications included beta blockers (85%), ACE inhibitors or ARBs (71%), ARNIs (9%) and SGLT-2 inhibitors (14%). Additional baseline details have been previously published and are incorporated by reference (Scott Solomon et al., N Engl J Med. 2024 Oct. 24; 391 (16): 1475-1485. doi: 10.1056/NEJMoa2407107.).

### Outcomes

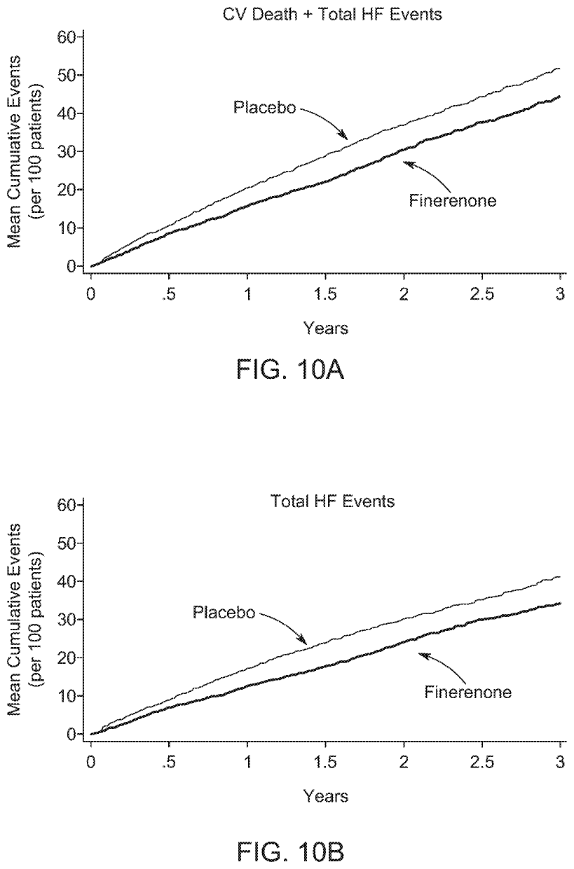

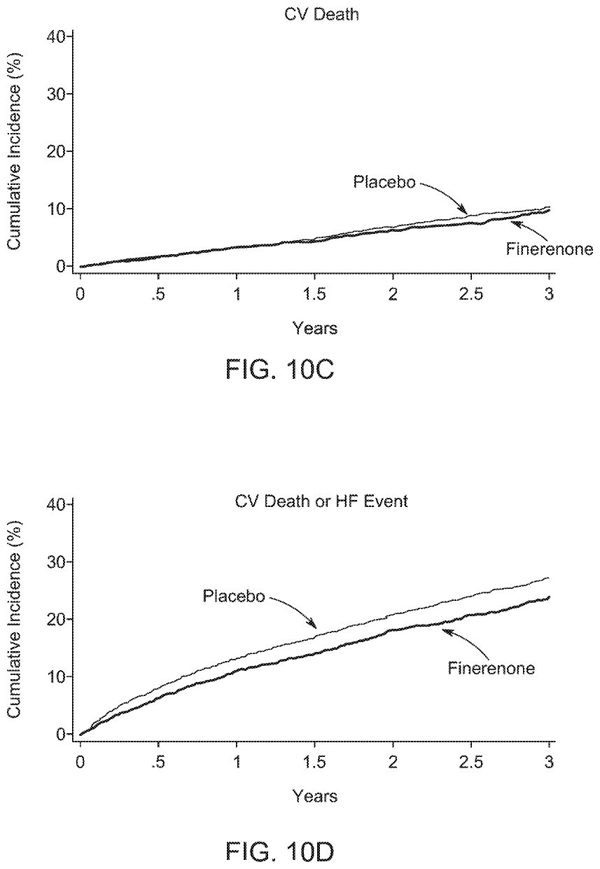

There were 1,083 primary events (842 heart failure events and 242 deaths from cardiovascular causes) in 624 patients in the finerenone group and 1,283 primary events (1,024 heart failure events and 260 deaths for cardiovascular causes) in 719 patients in the placebo group (rate ratio, 0.84; 95% confidence interval [CI], 0.74 to 0.95; P=0.007). (Table 20 and FIG. 10).

TABLE 20
Primary and Secondary Outcomes.*

| | Finerenone | | Placebo | | Ratio or Difference |
|---|---|---|---|---|---|
| Outcome | (N = 3,003) | | (N = 2,998) | | (95% CI) |
| Primary composite outcome and | | | | | |
| components | | | | | |
| Total worsening heart failure events and | | | | | RR, 0.84 (0.74- |
| death from cardiovascular causes | | | | | 0.95), p = 0.007 |
| Total no. of events** | 1,083 | | 1,283 | | |
| Rate per 100 patient-yr | 14.9 | | 17.7 | | |
| Total no. of worsening heart failure events** | 842 | | 1,024 | | RR, 0.82 (0.71- |
| | | | | | 0.94), p = 0.006 |
| Death from cardiovascular causes - no. (%) | 242 | (8.1%) | 260 | (8.7%) | HR, 0.93 (0.78-1.11) |
| Secondary outcomes | | | | | |
| Change in KCCQ clinical summary score at | +8.0 ± 0.3 | | +6.4 ± 0.3 | | +1.6 (0.8-2.3), |
| 6, 9 and 12 mo (least squares mean change) | | | | | p < 0.001 |
| Improvement in NYHA class from | 557/3,002 | (18.6%) | 553/2,998 | (18.4%) | OR, 1.01 (0.88-1.15) |
| baseline to 12 mo - no./total no. | | | | | |
| (%) | | | | | |
| Kidney composite outcome - no of events | 75 | (2.5%) | 55 | (1.8%) | HR, 1.33 (0.94-1.89) |
| (%)§ | | | | | |
| Death from any cause - no of events (%) | 491 | (16.4%) | 522 | (17.4%) | HR, 0.93 (0.83-1.06) |
| First worsening heart failure event or | 624 | (20.8%) | 719 | (24.0%) | HR, 0.84 (0.76-0.94) |
| death from cardiovascular causes | | | | | |

*HR denotes hazard ratio, OR odds ratio, and RR rate ratio.
**one patient in each group was reported as having a heart failure event on the same day as a cardiovascular death and was counted as only one composite event in the primary analysis
§Kidney composite outcome was defined as a sustained 50% or greater decline in eGFR, a sustained decline in eGFR to <15 ml/min/1.73 m² or initiation of chronic dialysis or kidney transplant

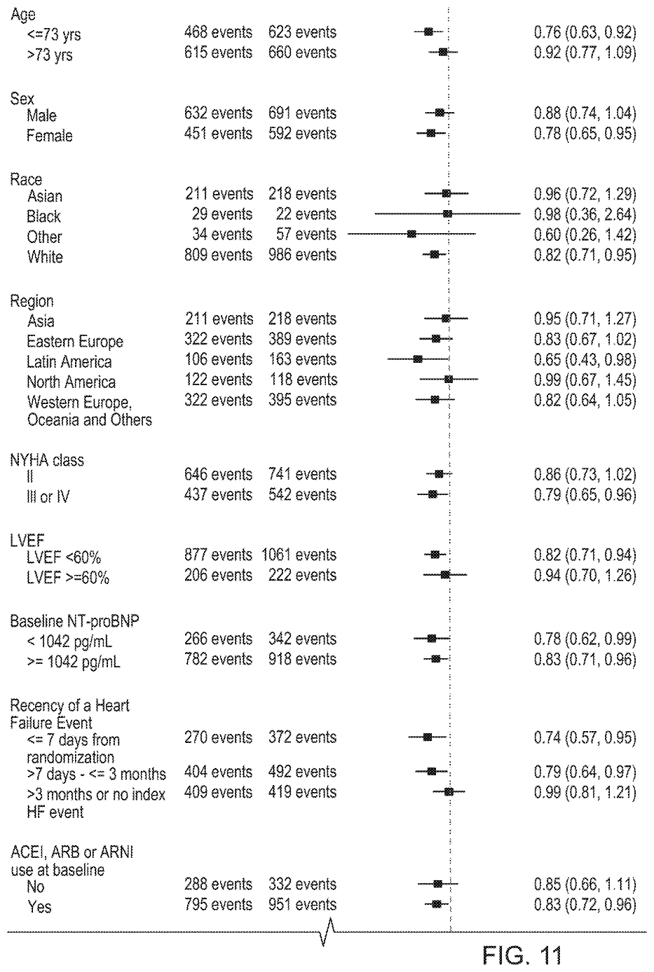

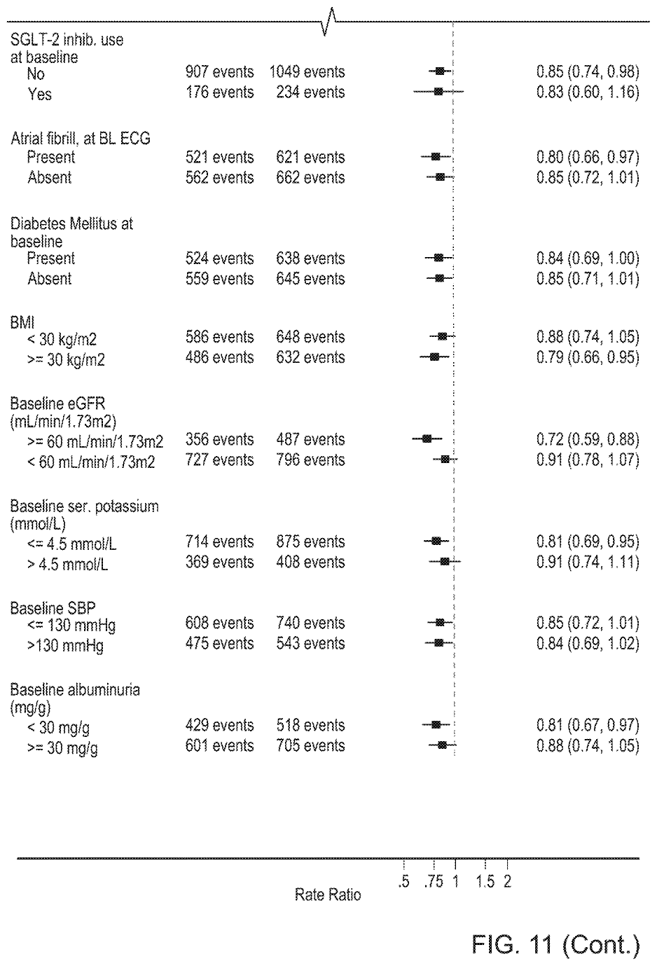

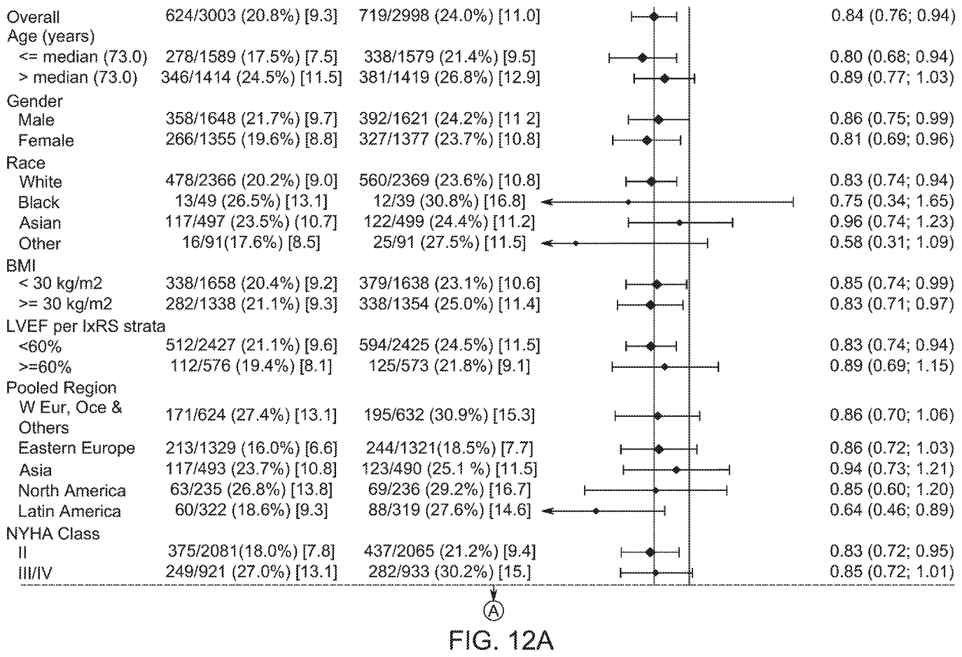

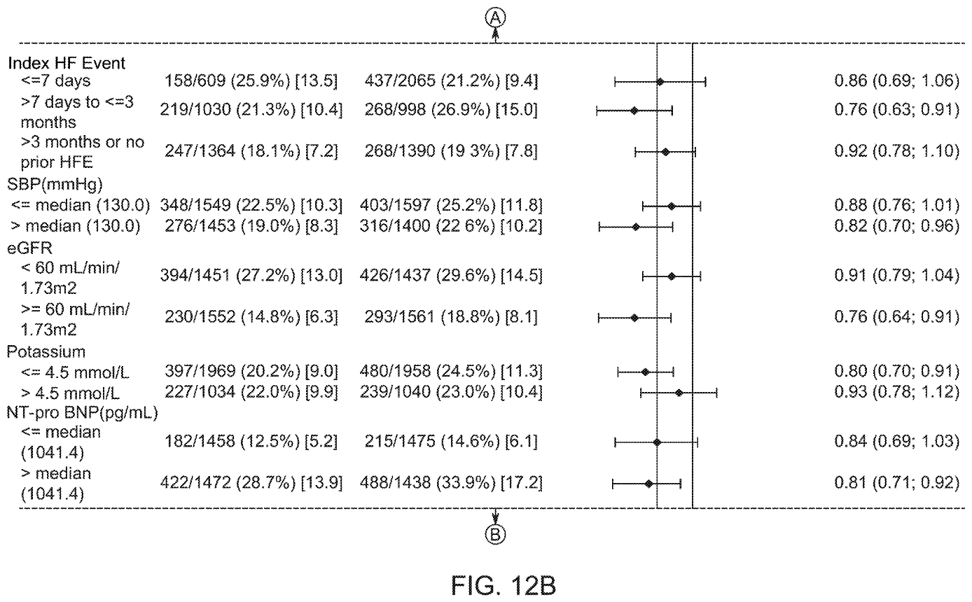

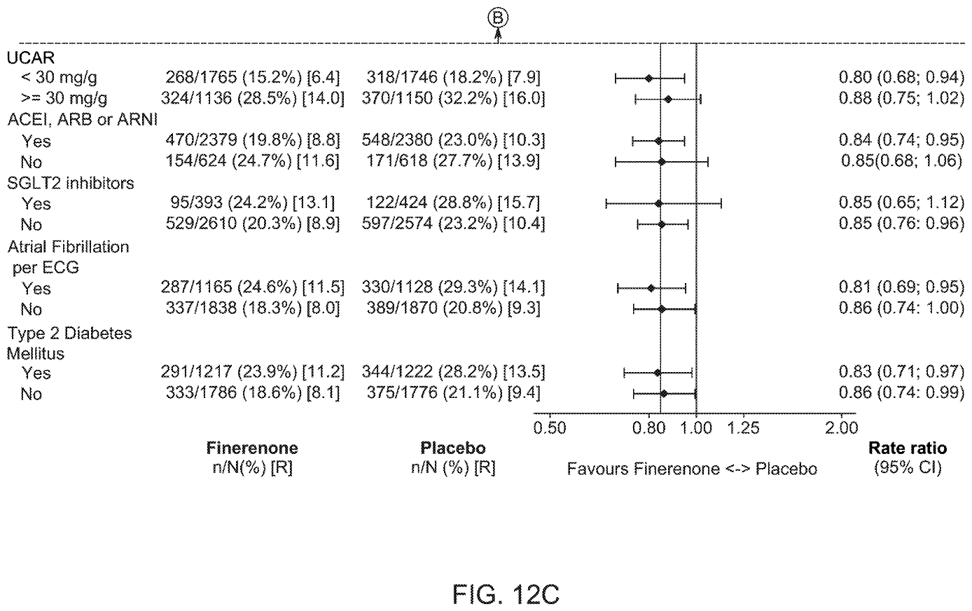

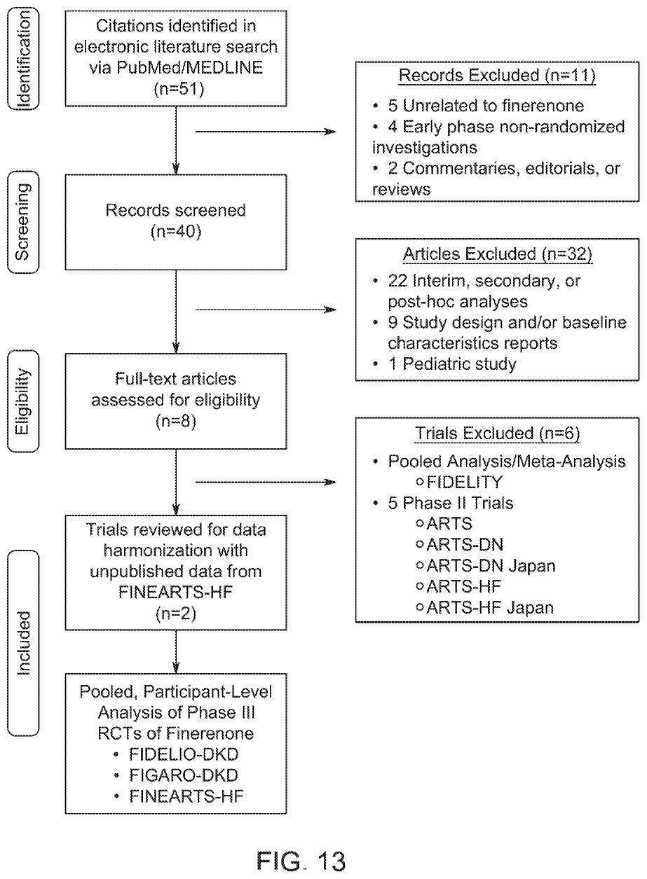

There were 842 and 1024 total heart failure events respectively in the finerenone and placebo groups (rate ratio, 0.82; 95% confidence interval [CI], 0.71 to 0.94, p=0.006). The first occurrence of cardiovascular death or heart failure event was also reduced in patients receiving finerenone compared with placebo (HR=0.84, 95% CI 0.76-0.94). The incidence of death from cardiovascular causes was 8.1% and 8.7%, respectively (hazard ratio, 0.93; 95% CI, 0.78 to 1.11). The primary outcome was consistent in all 17 prespecified subgroups (FIG. 11 and FIGS. 12A-12C), including based on ejection fraction or baseline use of SGLT2 inhibitors.

At Months 6, 9 and 12, there was a mean change from baseline in the KCCQ total symptoms score of 8.0 points in the finerenone group and 6.4 points in the placebo group (between-group difference 1.6 points; 95% CI, 0.8 to 2.3, p<0.001). NYHA class at 12 months improved in 557 patients (18.6%) in the finerenone group compared with 553 patients (18.4%) in the placebo group (odds ratio for improvement, 1.01; 95% CI, 0.88 to 1.15). The kidney composite outcome occurred in 75 patients (2.5%) in the finerenone group and 55 patients (1.8%) in the placebo group (hazard ratio, 1.33; 95% CI, 0.94 to 1.89). Death from any cause occurred in 491 patients (16.4%) in the finerenone group and 522 patients (17.4%) in the placebo group (hazard ratio, 0.93; 95% CI, 0.83 to 1.06).

Among those who received study drug, 611 patients (20.4%) in the finerenone group and 616 patients (20.6%) in the placebo group discontinued the trial drug for reasons other than death. At the end of study visit, among the patients who were continuing therapy, 68.4% in finerenone group were taking the individualized target dose, as compared with 78.4% in the placebo group. Mean daily dose of finerenone was 16 mg and 32 mg for patients with baseline eGFR≤60 ml/min/1.73 m² and >60 ml/min/1.73 m², respectively, with placebo patients receiving 17 mg and 34 mg of study drug, respectively.

Serious adverse events were similar between treatment groups (Table 21, Table 22).

TABLE 21
Treatment Emergent Safety Outcomes *.

| | Finerenone | | Placebo | |
|---|---|---|---|---|
| Event | (N = 2,993) | | (N = 2,993) | |
| Any Serious adverse event | 1,157 | (38.7%) | 1,213 | (40.5%) |
| Serum creatinine ≥3.0 mg/dl | 57 | (2.0%) | 34 | (1.2%) |
| Serum potassium | | | | |
| >5.5 mmol/liter | 413 | (14.3%) | 199 | (6.9%) |
| >6.0 mmol/liter | 86 | (3.0%) | 41 | (1.4%) |
| <3.5 mmol/liter | 127 | (4.4%) | 281 | (9.7%) |
| Investigator-Reported Hyperkalemia | 289 | (9.7%) | 125 | (4.2%) |
| Hyperkalemia leading to Hospitalization | 16 | (0.5%) | 6 | (0.2%) |
| Hyperkalemia leading to Death | 0 | (0%) | 0 | (0%) |
| Systolic blood pressure <100 mm Hg | 538 | (18.5%) | 361 | (12.4%) |

* Treatment emergent defined as all safety outcomes that occurred in patients who received at least one dose of study drug and up until 3 days following permanent discontinuation. All safety analyses were restricted to the 5,986 patients who received at least one dose of study drug. Counts of patients with abnormal creatinine, potassium, and SBP values were further restricted to patients with at least one treatment-emergent assessment (n = 5,785, 5,787, and 5,815 respectively)

TABLE 22
All adverse events during randomized
treatment through the end of study

| | Finerenone | | Placebo | |
|---|---|---|---|---|
| Event | (N = 2,993) | | (N = 2,993) | |
| Any Serious adverse event | 1,359 | (45.4%) | 1,378 | (46.0%) |
| Serum creatinine ≥3.0 mg/dl | 77 | (2.6%) | 45 | (1.5%) |
| Scrum potassium | | | | |
| >5.5 mmol/liter | 426 | (14.6%) | 207 | (7.1%) |
| >6.0 mmol/liter | 90 | (3.1%) | 44 | (1.5%) |
| <3.5 mmol/liter | 145 | (5.0%) | 299 | (10.3%) |
| Investigator-Reported Hyperkalemia | 311 | (10.4%) | 136 | (4.5%) |
| Hyperkalemia leading to Hospitalization | 24 | (0.8%) | 7 | (0.2%) |
| Hyperkalemia leading to Death | 0 | (0%) | 0 | (0%) |
| Systolic blood pressure <100 mm Hg | 556 | (19.0%) | 374 | (12.7%) |

* Defined as all safety events that occurred in patients who received at least one dose of study drug and up until end of study data collection. All safety analyses were restricted to the 5,986 patients who received at least one dose of study drug. Counts of patients with abnormal creatinine, potassium, and SBP values were further restricted to patients with at least one post-baseline assessment (n = 5,849, 5,836, and 5,869 respectively)

At Months 6, 9 and 12, there was a mean change from baseline in the KCCQ total symptoms score of 8.0 points in the finerenone group and 6.4 points in the placebo group (between-group difference 1.6 points; 95% CI, 0.8 to 2.3, p<0.001). NYHA class at 12 months improved in 557 patients (18.6%) in the finerenone group compared with 553 patients (18.4%) in the placebo group (odds ratio for improvement, 1.01; 95% CI, 0.88 to 1.15). The kidney composite outcome occurred in 75 patients (2.5%) in the finerenone group and 55 patients (1.8%) in the placebo group (hazard ratio, 1.33; 95% CI, 0.94 to 1.89). Death from any cause occurred in 491 patients (16.4%) in the finerenone group and 522 patients (17.4%) in the placebo group (hazard ratio, 0.93; 95% CI, 0.83 to 1.06).

Among those who received study drug, 611 patients (20.4%) in the finerenone group and 616 patients (20.6%) in the placebo group discontinued the trial drug for reasons other than death. At the end of study visit, among the patients who were continuing therapy, 68.4% in finerenone group were taking the individualized target dose, as compared with 78.4% in the placebo group. Mean daily dose of finerenone was 16 mg and 32 mg for patients with baseline eGFR ≤60 ml/min/1.73 m² and >60 ml/min/1.73 m², respectively, with placebo patients receiving 17 mg and 34 mg of study drug, respectively.

Serious adverse events were similar between treatment groups (Table 21, Table 22). Patients in the finerenone group were more likely to have increases in creatinine and potassium levels than those in the placebo group, although potassium values greater than 6.0 mmol/L were rare as were hyperkalemia episodes leading to hospitalization and there were no reported fatal episodes of hyperkalemia. Rates of decreased potassium levels (hypokalemia) by laboratory assessment or investigator report were less frequent with finerenone. The mean systolic blood pressure at 6 months was 3.4 mm Hg (95% CI, 2.6 to 4.2) lower in the finerenone group than in the placebo group. The most frequent adverse events are summarized in Table 23.

TABLE 23
Serious Adverse Events, Adverse Events Leading
to Discontinuation, and Other Adverse Events of Interest
Table 23.1: Number of subjects with a treatment-emergent*
SAE (>=0.5% in Finerenone group), by preferred term

| | Finerenone | Placebo | |
|---|---|---|---|
| | n = 2,993 | n = 2,993 | |
| Pneumonia | 87 (2.9%) | 110 | (3.7%) |
| Atrial fibrillation | 77 (2.6%) | 74 | (2.5%) |
| COVID-19 | 69 (2.3%) | 71 | (2.4%) |
| Acute kidney injury | 54 (1.8%) | 28 | (0.9%) |
| Death | 29 (1.0%) | 52 | (1.7%) |
| Angina unstable | 38 (1.3%) | 36 | (1.2%) |
| Anaemia | 34 (1.1%) | 31 | (1.0%) |
| COVID-19 pneumonia | 28 (0.9%) | 30 | (1.0%) |
| Urinary tract infection | 29 (1.0%) | 26 | (0.9%) |
| Syncope | 25 (0.8%) | 22 | (0.7%) |
| Chest pain | 23 (0.8%) | 22 | (0.7%) |
| Chronic obstructive pulmonary disease | 18 (0.6%) | 27 | (0.9%) |
| Angina pectoris | 17 (0.6%) | 26 | (0.9%) |
| Cellulitis | 17 (0.6%) | 24 | (0.8%) |
| Sepsis | 25 (0.8%) | 16 | (0.5%) |
| Coronary artery disease | 20 (0.7%) | 17 | (0.6%) |
| Cardiac failure | 18 (0.6%) | 18 | (0.6%) |
| Sudden death | 16 (0.5%) | 17 | (0.6%) |
| Femur fracture | 15 (0.5%) | 17 | (0.6%) |
| Fall | 15 (0.5%) | 14 | (0.5%) |
| Gastrointestinal haemorrhage | 17 (0.6%) | 8 | (0.3%) |
| Hyperkalemia | 19 (0.6%) | 6 | (0.2%) |

*Treatment emergent SAE is defined as any event occurring after a patient has received one dose of study drug and within 3 days of permanent discontinuation

TABLE 23.2
Number of subjects with AE of death or leading to death
(≥0.2% in Finerenone group), by preferred term.

| | Finerenone | | Placebo | |
|---|---|---|---|---|
| | n = 2,993 | | n = 2,993 | |
| Death | 70 | (2.3%) | 97 | (3.2%) |
| Pneumonia | 27 | (0.9%) | 23 | (0.8%) |
| Sudden death | 20 | (0.7%) | 26 | (0.9%) |
| Sudden cardiac death | 18 | (0.6%) | 19 | (0.6%) |
| Sepsis | 19 | (0.6%) | 7 | (0.2%) |
| Septic shock | 12 | (0.4%) | 12 | (0.4%) |
| COVID-19 | 7 | (0.2%) | 17 | (0.6%) |
| Cardiac arrest | 12 | (0.4%) | 12 | (0.4%) |
| COVID-19 pneumonia | 9 | (0.3%) | 10 | (0.3%) |
| Cardiac failure | 6 | (0.2%) | 12 | (0.4%) |
| Respiratory failure | 7 | (0.2%) | 4 | (0.1%) |
| Lung neoplasm malignant | 7 | (0.2%) | 3 | (0.1%) |

TABLE 23.3
Number of subjects with treatment-emergent AE leading to treatment
discontinuation (≥0.1% in Finerenone group), by preferred term.

| | |
|---|---|
| Finerenone | Placebo |
| n = 2,993 | n = 2,993 |

| | | | |
|---|---|---|---|
| Hyperkalaemia | 12 | (0.4%) | 5 (0.2%) |
| Renal impairment | 5 | (0.2%) | 4 (0.1%) |
| Diarrhoea | 4 | (0.1%) | 4 (0.1%) |
| Nausea | 3 | (0.1%) | 3 (0.1%) |
| Asthenia | 5 | (0.2%) | 0 (0.0%) |
| Dyspnoea | 3 | (0.1%) | 1 (0.0%) |
| Hypotension | 3 | (0.1%) | 0 (0.0%) |

### DISCUSSION

In this randomized, placebo-controlled trial in patients with heart failure and a mildly reduced or preserved ejection fraction, finerenone resulted in a lower risk of the primary composite outcome of cardiovascular death and total worsening heart failure events, compared with placebo. Each of the components of this composite outcome was less common in the finerenone group than in the placebo group, and results were consistent in all pre-specified subgroups. Finerenone reduced both total and first worsening heart failure events, and modestly improved patient-reported health status as measured by the Kansas City Cardiomyopathy Questionnaire total symptom score. Overall serious adverse events were similar between treatment groups, while those receiving finerenone experienced more hyperkalemia, as evidenced by laboratory measurements or investigator-reported events, and more elevation in serum creatinine, but hypokalemia was less frequent with finerenone.

TOPCAT was the only other outcomes trial to test mineralocorticoid antagonism in patients with heart failure and mildly reduced or preserved ejection fraction. In that trial, the steroidal MRA spironolactone did not reduce the primary endpoint although post hoc analyses revealed differential event rates, study drug use, and treatment effects according to geographic region. Thus, the results of FINEARTS-HF with the nonsteroidal MRA finerenone provide the first definitive evidence that an MRA is beneficial in these patients and will inform clinicians treating these patients and potentially future guidelines.

While we did not observe any heterogeneity in the treatment effect for the primary outcome in any of the pre-specified subgroups, suggestions of greater benefit in patients with a more recent heart failure event were consistent with findings in other trials. Importantly, there was no evidence of heterogeneity in relation to background use of a sodium-glucose co-transporter 2 inhibitor, the only treatment with a strong guideline recommendation in this population, although this recommendation was not introduced until late in the conduct of the FINEARTS-HF trial. Finerenone treatment also led to a modest improvement in a patient-reported outcome (KCCQ-Total Symptom Score) which was of a similar magnitude to that noted with other agents in other outcomes trials in heart failure (e.g., PARGAGON, DELIVER, EMPEROR). However, finerenone did not improve NYHA functional class, a physician-reported assessment of functional status. This may reflect the relative insensitivity of the four-category NYHA classification compared to the KCCQ-Total Symptom Score and this discordance has been noted in other trials.

Finerenone did not reduce the occurrence of the secondary composite kidney outcome in this population at low risk for kidney disease progression with low levels of albuminuria and low total number of kidney events. This finding is in contrast to FIDELITY, conducted in T2D patients with CKD and albuminuria at high risk of kidney disease progression, where finerenone reduced a similar kidney endpoint. However, the response in eGFR after initiation of a MRA may be different between patients with heart failure compared to patients with diabetic kidney disease. Other inhibitors of the renin-angiotensin-aldosterone system have failed to show kidney benefits in patients with heart failure but demonstrated a nephroprotective effect in patients with chronic kidney disease and type 2 diabetes. With respect to safety, in line with previous trials with MRAs, hyperkalemia, whether assessed as a measured laboratory value or as an investigator-reported adverse event, was more common in the finerenone group than in the placebo group, although only 0.5% of patients in the finerenone group, and 0.2% of patients in the placebo group had hyperkalemia leading to hospitalization and no patients experienced hyperkalemia leading to death.

Among patients with heart failure and a mildly reduced or preserved ejection fraction, finerenone reduced the risk of the primary composite outcome of cardiovascular death and total heart failure events, reduced total heart failure events, and improved patient-reported health status. These findings were consistent across prespecified subgroups. Hyperkalemia was more common, and hypokalemia less common, in those receiving finerenone. These data support the use of finerenone in patients with heart failure with mildly reduced or preserved ejection fraction.

Finerenone demonstrated a statistically significant 16% relative risk reduction in the composite CV outcome (number of CV deaths and HF events) in patients with HF and LVEF ≥40%, compared to placebo.

The effects of finerenone on the CV composite outcome were consistent across a broad population, regardless of baseline use of an SGLT-2i or LVEF status.

Finerenone demonstrated significant benefits in the secondary efficacy endpoints of total HF events and patient-reported health status.

Finerenone was well tolerated, confirming the well-known safety profile, with a comparable incidence of treatment-emergent SAEs (serious adverse events) to placebo.

### Example 14—FINE-HEART: An Integrated Pooled Analysis of Finerenone across Three Phase III Trials of Heart Failure and Chronic Kidney Disease with Type 2 Diabetes

Cardiovascular-kidney-metabolic syndrome (CKM) is a health disorder that connects cardiovascular diseases, chronic kidney disease and diabetes. The non-steroidal mineralocorticoid receptor antagonist, finerenone, has been studied in three prospective randomized trials of patients with CKM. However, none of these trials was individually powered to evaluate treatment effects on mortality outcomes.

Objective: To summarize the efficacy and safety of finerenone on cardiovascular, kidney, and mortality outcomes in all completed phase III clinical trials conducted to date.

Participant-level pooled analysis of 3 phase III global, multicenter, double-blind, placebo-controlled randomized clinical trials

Chronic kidney disease and type 2 diabetes (FIDELIO-DKD and FIGARO-DKD) and heart failure with mildly reduced or preserved ejection fraction (FINEARTS-HF)

The primary endpoint was cardiovascular death (excluding undetermined deaths). A prespecified sensitivity analysis considered deaths of undetermined causes to be cardiovascular deaths. Secondary endpoints included all cause death, all-cause hospitalization, HF hospitalization, and a composite kidney outcome (50% or greater sustained decline in estimated glomerular filtration rate, kidney failure, or death from kidney causes).

The 3 trials included 18,991 participants (mean age 67±10 years; 35% women). During 2.9 years median follow-up, cardiovascular death occurred in 421 (4.4%) assigned to finerenone and 471 (5.0%) assigned to placebo (HR 0.89; 95% CI 0.78-1.01; P=0.076). Consistent findings were observed in the prespecified sensitivity analysis including both cardiovascular deaths and undetermined deaths (HR 0.88; 95% CI 0.79-0.98; P=0.025). Death from any cause occurred in 1,042 (11.0%) participants in the finerenone arm and 1,136 (12.0%) in the placebo arm (HR 0.91; 95% CI 0.84-0.99; P=0.027). Finerenone further reduced the risk of HF hospitalization (HR 0.83; 95% CI 0.75-0.92; P<0.001) and the composite kidney outcome (HR 0.80; 95% CI 0.72-0.90; P<0.001). Treatment effects on cardiovascular death were generally consistent across the 16 subgroups examined. Serious adverse events were lower with finerenone than placebo (34.6% vs. 36.6%), but hyperkalemia was increased with finerenone.

Conclusions and Relevance: While this pooled analysis failed to demonstrate significant reductions in cardiovascular death, finerenone was associated with significantly lower deaths of any cause, cardiovascular events, and kidney outcomes. The totality of the evidence thus supports the disease-modifying potential of finerenone in broad, high-risk patient populations encompassing cardiovascular, kidney, and metabolic diseases. ClinicalTrials.gov Trial Registration: NCT04435626; NCT02540993; NCT02545049. PROSPERO Registration of Pooled Analysis: CRD42024570467.

The efficacy and safety of the non-steroidal mineralocorticoid receptor antagonist finerenone across a broad range of people with cardio-kidney-metabolic conditions: We conducted a pooled, participant-level analysis of all three phase III clinical trials of finerenone completed to date. While finerenone did not significantly reduce the primary endpoint of cardiovascular death, this result was sensitive to the definition of cardiovascular death and all-cause death was significantly lower with finerenone as were cardiovascular and kidney events. The totality of evidence supports the addition of finerenone as a foundational pillar of care in broad, high-risk patient populations encompassing cardiovascular, kidney, and metabolic diseases.

### In Detail:

### Methods

Search Strategy and Trial Selection. We conducted a participant-level pooled analysis of 2 trials of CKD and type 2 diabetes (FIDELIO-DKD [Finerenone in Reducing Kidney Failure and Disease Progression in Diabetic Kidney Disease; NCT02540993] and FIGARO-DKD [Finerenone in Reducing Cardiovascular Mortality and Morbidity in Diabetic Kidney Disease; NCT02545049]) and a trial of patients with HF (FINEARTS-HF [FINerenone trial to investigate Efficacy and sAfety superioR to placebo in paTientS with Heart Failure; NCT04435626]) that included patients with and without diabetes. The designs 18-20 and primary results15-17 of each of the 3 trials have been published. Key design elements of each of the trials are summarized in Table 24.

TABLE 24
Key Study Design Features

| | FINEARTS-HF | FIDELIO-DKD and FIGARO-DKD |
|---|---|---|
| Validly Randomized | 6,001 | 12,990 |
| Countries | 37 | 48 |
| Patient population | HF with mildly reduced or preserved | CKD and T2D |
| | ejection fraction | |
| Inclusion criteria | Adults (≥40 years) | Adults (≥18 years old) |
| | Symptomatic HF | T2D |
| | LVEF ≥40% | UACR ≥30 mg/g |
| | Elevation natriuretic peptides | Maximally tolerated renin- |
| | Structural heart disease | angiotensin system inhibitors |
| | Recent diuretic use | |
| Exclusion criteria | Potassium ≤5.0 mmol/L | Potassium ≤4.8 mmol/L |
| Dosage and titration | eGFR ≤60: 10 up to 20 mg | eGFR <60: 10 up to 20 mg |
| | eGFR >60: 20 up to 40 mg (potentially | eGFR ≥60: 20 mg |
| | down to 10 mg) | (potentially down to 10 mg) |
| Study duration | 2.6 years | 2.6 years (FIDELIO-DKD) |
| | | 3.4 years (FIGARO-DKD) |

Abbreviations:
CKD = chronic kidney disease;
eGFR = estimated glomerular filtration rate;
HF = heart failure;
T2D = type 2 diabetes;
UACR = urine albumin-to-creatinine ratio

We further conducted a systematic review of the literature using PubMed and MEDLINE to ensure that other relevant trials were not missed. Data from the FINEARTS-HF trial were unpublished at the time of analysis and included for this analysis.

Design of FIDELIO-DKD and FIGARO-DKD. In brief, both FIDELIO-DKD and FIGARO-DKD trials enrolled adults (≥18 years old) with type 2 diabetes and CKD across 48 countries. FIDELIO-DKD required urinary albumin-to-creatinine ratio (UACR) of 30 to <300 mg/g, estimated glomerular filtration rate (eGFR) of 25 to <60 mL/min/1.73 m², and a history of diabetic retinopathy or a UACR of 300 to 5,000 mg/g and eGFR of 25 to <75 mL/min/1.73 m². FIGARO-DKD required a UACR of 30 to <300 mg/g and an eGFR of 25 to 90 mL/min/1.73 m² or a UACR of 300 to 5,000 mg/g and an eGFR ≥60 mL/min/1.73 m². Both trials required a serum potassium ≤4.8 mmol/L for enrollment. Renin-angiotensin system inhibitor use and dosing was optimized prior to screening during run-in phases (lasting 4 to 16 weeks) in both trials. Patients with symptomatic HF with reduced ejection fraction were excluded, but those with HF and higher EF were eligible.

Design of FINEARTS-HF. FINEARTS-HF enrolled adults (≥40 years) with symptomatic HF with mildly reduced or preserved ejection fraction across 37 countries. Key inclusion criteria included left ventricular ejection fraction ≥40%, elevated natriuretic peptides (adjusted based on atrial fibrillation status and clinical setting of screening), evidence of structural heart disease and recent diuretic use for at least 30 days. Patients were required to have an eGFR ≥25 mL/min/1.73 m² and a serum potassium level ≤5.0 mmol/L for enrollment. Participants could be enrolled regardless of clinical care setting (whether hospitalized, recently hospitalized, or ambulatory).

All participants were randomly allocated to finerenone or placebo with initial dosing determined based on kidney function. The initial dose was 10 mg for patients with an eGFR <60 mL/min/1.73 m², titrated up to a target dose of 20 mg once daily as tolerated. Participants with an eGFR ≥60 mL/min/1.73 m² were started at the target dose of 20 mg once daily. In FINEARTS-HF, participants with an eGFR >60 mL/min/1.73 m² were started on 20 mg and could be titrated up to 40 mg once daily as tolerated, while 20 mg was the target dose for patients with eGFR ≤60 ml/min/1.73 m². The trial protocols were approved by ethics committees or institutional review boards at all participating sites and all patients provided explicit written informed consent.

FINE-HEART Pooled Analysis Endpoints. Individual participant-level data were accessed and pooled with harmonized data elements for baseline characteristics and clinical outcomes (Table 25).

TABLE 25
Harmonization of Key Outcome Measures and Their Assessment Across Trials

| Data Element | Harmonization Strategy |
|---|---|
| Primary Efficacy | In the FIDELIO-DKD and FIGARO-DKD trials, deaths of |
| Endpoint of CV | undetermined causes were considered cardiovascular. In the |
| Death | FINEARTS-HF trial, deaths of undetermined causes were not |
| | considered cardiovascular. |
| | For the pooled FINE-HEART analysis, the primary endpoint |
| | was CV death alone (excluding deaths of undetermined causes). |
| | Prespecified sensitivity analyses considered all undetermined |
| | deaths as cardiovascular in etiology. |
| Stratification Factors | In the time to event analyses, stratification factors were |
| for Efficacy Models | individual trial and geographic region. The countries included |
| | in each geographic region varied by trial. |
| | For the purposes of FINE-HEART, geographic region was |
| | harmonized to the following designations: |
| | Western Europe, Oceania and Others: Australia, |
| | Austria, Belgium, Denmark, France, Germany, Ireland, |
| | Israel, Italy, Netherlands, New Zealand, Norway, |
| | Portugal, South Africa, Spain, Sweden, Switzerland, |
| | United Kingdom |
| | Eastern Europe: Bulgaria, Czechia, Finland, Greece, |
| | Hungary, Latvia, Lithuania, Poland, Romania, Russia, |
| | Slovakia, Turkey, Ukraine |
| | Asia: China, Hong Kong, India, Japan, Malaysia, |
| | Philippines, Singapore, South Korea, Taiwan, Thailand, |
| | Vietnam |
| | North America: Canada, Puerto Rico, United States of |
| | America |
| | Latin America: Argentina, Brazil, Chile, Colombia, |
| | Mexico |
| Kidney Composite | The primary kidney composite endpoint specified in the |
| Endpoint | FIDELITY program (which encompassed FIDELIO-DKD and |
| | FIGARO-DKD) was time to first onset of kidney failure, |
| | sustained ≥57% decrease in eGFR from baseline over ≥4 weeks, |
| | or death from kidney causes. The kidney composite endpoint (a |
| | secondary endpoint) in the FINEARTS-HF trial was time to first |
| | onset of kidney failure or sustained ≥50% decrease in eGFR |
| | from baseline over ≥4 weeks. Kidney outcomes were |
| | adjudicated by independent clinical events committees in each |
| | of the 3 trials. |
| | In FINE-HEART, the kidney composite endpoint selected was |
| | time to first onset of kidney failure, sustained ≥50% decrease in |
| | eGFR from baseline over ≥4 weeks, or death from kidney |
| | causes. |
| Treatment Emergent | The definition of treatment emergent safety events slightly |
| Adverse Events | differed across trials. Whereas FINEARTS-HF considered |
| | treatment emergent adverse events as any adverse event |
| | occurring in a patient who received at least one dose of study |
| | drug and up until 3 days of permanent discontinuation, |
| | FIDELIO-DKD and FIGARO-DKD considered the same period |
| | but excluded adverse events while a participant was on |
| | temporary interruption of study drug. |
| | To remain conservative in our safety accounting, in FINE- |
| | HEART, we considered all adverse events occurring in any |
| | patient who has received at least one dose of study drug and |
| | within 3 days of permanent discontinuation, including during |
| | periods of temporary drug interruption. |

All participants randomized in each of the three trials were considered for this pooled analysis with only patients with critical Good Clinical Practice violations excluded. All efficacy outcomes were analyzed in randomized patients under intention-to-treat principles, while all safety outcomes were analyzed in randomized patients who had taken at least one dose of the study drug. The primary endpoint (cardiovascular death) was assessed across key subgroups including age, sex, race, region, baseline body mass index, baseline systolic blood pressure, Kidney Disease: Improving Global Outcomes (KDIGO) risk, baseline serum potassium levels, baseline eGFR, baseline UACR, history of HF, history of diabetes, presence of CKD, degree of cardio-kidney-metabolic overlap, and baseline use of sodium-glucose-co-transporter-2 inhibitors (SGLT2i) or glucagon-like-peptide-1 receptor agonists (GLP-1RA).

The prespecified primary endpoint for FINE-HEART was time to cardiovascular death. The definition of the endpoint cardiovascular death differed slightly between the three trials and was harmonized for FINE-HEART as time to cardiovascular death (excluding undetermined deaths); Table 25. All deaths were adjudicated by independent clinical endpoint committees in each of the respective trials included in this pooled analysis. Other prespecified endpoints included a kidney composite endpoint (defined as a sustained decline in eGFR to ≥50% from baseline, sustained decrease in eGFR to <15 mL/min/1.73 m², end-stage kidney disease, and death due to kidney causes), HF hospitalization, composite of cardiovascular death or HF hospitalization, new-onset atrial fibrillation, major adverse cardiovascular events (a composite of non-fatal myocardial infarction, non-fatal stroke, HF hospitalization, or cardiovascular death), all-cause death, and hospitalization for any reason. The composite of all-cause death or all-cause hospitalization was defined post hoc to describe the total burden or morbidity and mortality. Select treatment-emergent adverse events related to hyperkalemia, acute kidney injury, hypotension, and gynecomastia were also reported in the pooled population.

Statistical Analysis. All primary and secondary endpoints were analyzed as time-to-first endpoints and analyzed using a stratified Cox proportional hazards model including the study intervention group as a fixed effect and stratified by geographic region and individual trial (Table 25). All treatment effect estimates are presented as hazard ratios (HR) with associated 95% confidence intervals (CI). Select primary and secondary endpoints were additionally graphically displayed using Kaplan Meier methods. A prespecified sensitivity analysis was conducted for the primary endpoint which considered deaths of undetermined causes to be cardiovascular deaths in all three trials. Treatment effects on cardiovascular death were assessed across all prespecified subgroups. Incidence rates of cardiovascular death as a function of baseline eGFR and log-transformed UACR were estimated separately for each treatment arm using Poisson regression models, allowing for potentially non-linear relationships using restricted cubic splines with three knots. The treatment effect of finerenone was then estimated as the ratio of these two group-specific estimates.

The statistical analysis plan for this pooled analysis was prespecified and the protocol was prospectively registered in the International Prospective Register of Systematic Reviews (PROSPERO CRD42024570467) before unblinding the FINEARTS-HF trial. All 3 trials were assessed as high quality with a low risk of bias prior to pooling (Table 26).

TABLE 26
Risk of Bias Assessment Using the Revised Tool to
Assess Risk of Bias in Randomized Trials (RoB 2.0)

| | | | | Missing | Measurement | Reporting | |
|---|---|---|---|---|---|---|---|
| Study | | Randomization | Intervention | Outcome | of | of | Overall |
| Name | Year | Bias | Deviation | Data | Outcome | Outcome | Risk |
| FIDELIO- | 2020 | Low | Low | Low | Low | Low | Low |
| DKD | | | | | | | |
| FIGARO- | 2021 | Low | Low | Low | Low | Low | Low |
| DKD | | | | | | | |
| FINEARTS | 2024 | Low | Low | Low | Low | Low | Low |

The primary analyses, interpretation of the data, and initial manuscript drafting were conducted. Statistical analyses were conducted using STATA version 18.

### Results

Overall, FINE-HEART comprised 18,991 participants from these three trials. Baseline clinical profiles and treatment patterns are summarized for the overall pooled population (Table 27.1) and by individual trial (Table 27.2).

TABLE 27.1
Baseline Characteristics

| | Finerenone | Placebo |
|---|---|---|
| | n = 9,501 | n = 9,490 |
| Age | 67.0 ± 10.0 | 67.1 ± 10.2 |
| Female | 3,390 (35.7%) | 3,274 (34.5%) |
| Race^{a} | | |
| Asian | 1,910 (20.1%) | 1,946 (20.5%) |
| Black | 300 (3.2%) | 308 (3.2%) |
| Other | 476 (5.0%) | 447 (4.7%) |
| White | 6,815 (71.7%) | 6,789 (71.5%) |
| Region | | |
| Asia | 1,808 (19.0%) | 1,815 (19.1%) |
| Eastern Europe | 3,001 (31.6%) | 2,941 (31.0%) |
| Latin America | 1,041 (11.0%) | 1,034 (10.9%) |
| North America | 1,259 (13.3%) | 1,261 (13.3%) |
| Western Europe, Oceania and Others | 2,392 (25.2%) | 2,439 (25.7%) |
| Body Mass Index (kg/m²) | 30.9 ± 6.1 | 30.9 ± 6.0 |
| Systolic Blood Pressure (mmHg) | 134.5 ± 14.9 | 134.4 ± 15.0 |
| Potassium (mmol/L) | 4.4 ± 0.5 | 4.4 ± 0.5 |
| eGFR (mL/min/1.73 m²) | 58.9 ± 21.0 | 59.1 ± 21.3 |
| eGFR Category | | |
| <25 mL/min/1.73 m² | 100 (1.1%) | 94 (1.0%) |
| 25 to <45 mL/min/1.73 m² | 2,742 (28.9%) | 2,782 (29.3%) |
| 45 to <60 mL/min/1.73 m² | 2,513 (26.5%) | 2,469 (26.0%) |
| ≥60 mL/min/1.73 m² | 4,145 (43.6%) | 4,143 (43.7%) |
| UACR (mg/g) | 283 [46-836] | 293 [47-855] |
| Albuminuria Category | | |
| A1 (<30 mg/g) | 1,885 (20.1%) | 1,856 (19.8%) |
| A2 (30 to <300 mg/g) | 2,910 (31.0%) | 2,883 (30.7%) |
| A3 (≥300 mg/g) | 4,602 (49.0%) | 4,646 (49.5%) |
| Hemoglobin A1c (%) | 7.3 ± 1.4 | 7.3 ± 1.4 |
| AF on Electrocardiogram | 1,449 (15.3%) | 1,379 (14.5%) |
| History of HF^{b} | 3,488 (36.7%) | 3,520 (37.1%) |
| Baseline CKD^{c} | 7,949 (83.7%) | 7,929 (83.6%) |
| History of DM^{d} | 7,715 (81.2%) | 7,714 (81.3%) |
| Background Medication Use | | |
| Diuretics | 6,291 (66.2%) | 6,340 (66.8%) |
| ACEi/ARB/ARNI | 8,866 (93.3%) | 8,860 (93.4%) |
| Aspirin | 4,145 (43.6%) | 4,171 (44.0%) |
| Statins | 6,687 (70.4%) | 6,750 (71.1%) |
| SGLT-2 Inhibitors | 829 (8.7%) | 861 (9.1%) |
| GLP-1 Receptor Agonists | 576 (6.1%) | 534 (5.6%) |
| Potassium Lowering Therapies^{e} | 99 (1.0%) | 96 (1.0%) |

Abbreviations:
ACEi = angiotensin converting enzyme inhibitors;
AF = atrial fibrillation;
ARB = angiotensin II receptor blocker;
ARNI = angiotensin receptor neprilysin inhibitor;
CKD = chronic kidney disease;
DM = diabetes mellitus;
eGFR = estimated glomerular filtration rate;
GLP-1 = glucagon-like peptide-1 receptor agonist;
SGLT-2 = sodium-glucose co-transporter-2;
UACR = urine albumin-creatinine ratio
^{a}Represents self-reported race. Participants choosing not disclose race or who self-identified as multiple races are included in the “Other” category for descriptive purposes.
^{b}HF includes all participants in FINEARTS-HF and those with investigator-reported history of HF in the primary CKD outcomes trials (FIDELIO-DKD, FIGARO-DKD)
^{c}CKD includes all participants in the primary CKD outcomes trials (FIDELIO-DKD, FIGARO-DKD) and participants in FINEARTS-HF with baseline eGFR <60 mL/min/1.73 m²
^{d}Diabetes includes all participants in the primary CKD outcomes trials (FIDELIO-DKD, FIGARO-DKD) and those with a history of diabetes in FINEARTS-HF
^{e}Includes patiromer, sodium polystyrene sulfonate, calcium polystyrene sulfonate

TABLE 27.2
Baseline Characteristics by Trial

| | FIDELIO-DKD | FIGARO-DKD | FINEARTS-HF |
|---|---|---|---|
| | n = 5,662 | n = 7,328 | n = 6,001 |
| Age | 65.6 ± 9.0 | 64.2 ± 9.8 | 72.0 ± 9.6 |
| Female | 1,688 (29.8%) | 2,244 (30.6%) | 2,732 (45.5%) |
| Race^{a} | | | |
| Asian | 1,430 (25.3%) | 1,430 (19.5%) | 996 (16.6%) |
| Black | 262 (4.6%) | 258 (3.5%) | 88 (1.5%) |
| Other | 378 (6.7%) | 363 (5.0%) | 182 (3.0%) |
| White | 3,592 (63.4%) | 5,277 (72.0%) | 4,735 (78.9%) |
| Region | | | |
| Asia | 1,317 (23.3%) | 1,323 (18.1%) | 983 (16.4%) |
| Eastern Europe | 1,169 (20.6%) | 2,123 (29.0%) | 2,650 (44.2%) |
| Latin America | 593 (10.5%) | 841 (11.5%) | 641 (10.7%) |
| North America | 942 (16.6%) | 1,107 (15.1%) | 471 (7.8%) |
| Western Europe, Oceania and Others | 1,641 (29.0%) | 1,934 (26.4%) | 1,256 (20.9%) |
| Body Mass Index (kg/m²) | 31.1 ± 6.0 | 31.4 ± 6.0 | 29.9 ± 6.1 |
| Systolic Blood Pressure (mmHg) | 138.0 ± 14.4 | 135.7 ± 14.0 | 129.4 ± 15.3 |
| Potassium (mmol/L) | 4.4 ± 0.5 | 4.3 ± 0.4 | 4.4 ± 0.5 |
| eGFR (mL/min/1.73 m²) | 44.3 ± 12.6 | 67.8 ± 21.7 | 62.1 ± 19.7 |
| eGFR Category | | | |
| <25 mL/min/1.73 m² | 135 (2.4%) | 27 (0.4%) | 32 (0.5%) |
| 25 to <45 mL/min/1.73 m² | 2,973 (52.5%) | 1,251 (17.1%) | 1,300 (21.7%) |
| 45 to <60 mL/min/1.73 m² | 1,896 (33.5%) | 1,530 (20.9%) | 1,556 (25.9%) |
| ≥60 mL/min/1.73 m² | 656 (11.6%) | 4,519 (61.7%) | 3,113 (51.9%) |
| Baseline UACR (mg/g) | 853 [446-1636] | 309 [108-741] | 18 [7-67] |
| Albuminuria Category | | | |
| A1 (<30 mg/g) | 23 (0.4%) | 207 (2.8%) | 3,511 (60.6%) |
| A2 (30 to <300 mg/g) | 682 (12.1%) | 3,399 (46.4%) | 1,712 (29.5%) |
| A3 (≥300 mg/g) | 4,954 (87.5%) | 3,720 (50.8%) | 574 (9.9%) |
| Hemoglobin A1c (%) | 7.7 ± 1.3 | 7.7 ± 1.4 | 6.4 ± 1.2 |
| AF on Electrocardiogram | 233 (4.1%) | 302 (4.1%) | 2,293 (38.2%) |
| History of HF^{b} | 436 (7.7%) | 571 (7.8%) | 6,001 (100.0%) |
| Baseline CKD^{c} | 5,662 (100.0%) | 7,328 (100.0%) | 2,888 (48.1%) |
| History of DM^{d} | 5,662 (100.0%) | 7,328 (100.0%) | 2,439 (40.6%) |
| Background Medication Use | | | |
| Diuretics | 3,209 (56.7%) | 3,492 (47.7%) | 5,930 (98.8%) |
| ACEi/ARB/ARNI | 5,648 (99.8%) | 7,319 (99.9%) | 4,759 (79.3%) |
| Aspirin | 2,787 (49.2%) | 3,581 (48.9%) | 1,948 (32.5%) |
| Statins | 4,208 (74.3%) | 5,179 (70.7%) | 4,050 (67.5%) |
| SGLT-2 Inhibitors | 258 (4.6%) | 615 (8.4%) | 817 (13.6%) |
| GLP-1 Receptor Agonists | 393 (6.9%) | 550 (7.5%) | 167 (2.8%) |
| Potassium Lowering Therapies | 136 (2.4%) | 46 (0.6%) | 13 (0.2%) |

Abbreviations:
ACEi = angiotensin converting enzyme inhibitors;
AF = atrial fibrillation;
ARB = angiotensin II receptor blocker;
ARNI = angiotensin receptor neprilysin inhibitor;
CKD = chronic kidney disease;
DM = diabetes mellitus;
eGFR = estimated glomerular filtration rate;
GLP-1 = glucagon-like peptide-1 receptor agonist;
SGLT-2 = sodium-glucose co-transporter-2;
UACR = urine albumin-creatinine ratio
^{a}Represents self-reported race. Participants choosing not disclose race or who self-identified as multiple races are included in the “Other” category for descriptive purposes.
^{b}HF includes all participants in FINEARTS-HF and those with investigator-reported history of HF in the primary CKD outcomes trials (FIDELIO-DKD, FIGARO-DKD)
^{c}CKD includes all participants in the primary CKD outcomes trials (FIDELIO-DKD, FIGARO-DKD) and participants in FINEARTS-HF with baseline eGFR <60 mL/min/1.73 m²
^{d}Diabetes includes all participants in the primary CKD outcomes trials (FIDELIO-DKD, FIGARO-DKD) and those with a history of diabetes in FINEARTS-HF
^{e}Includes patiromer, sodium polystyrene sulfonate, calcium polystyrene

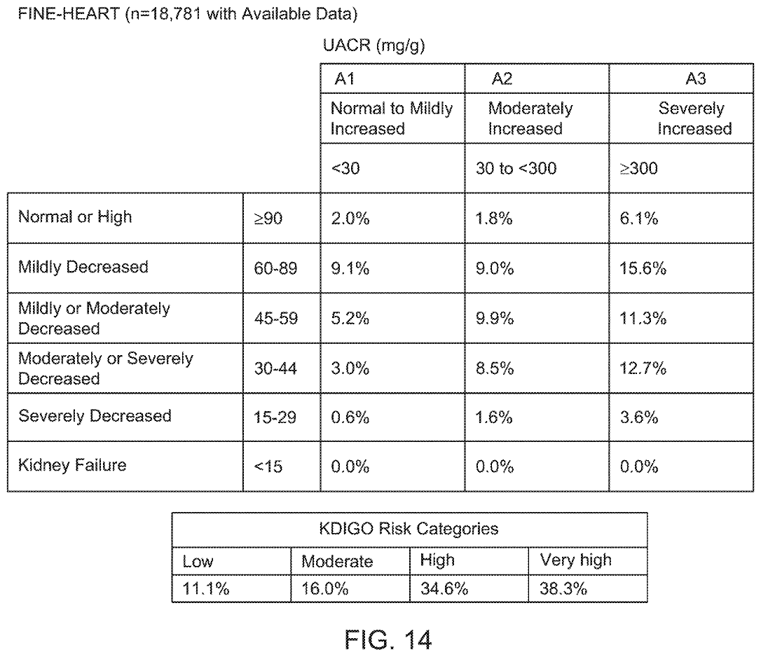

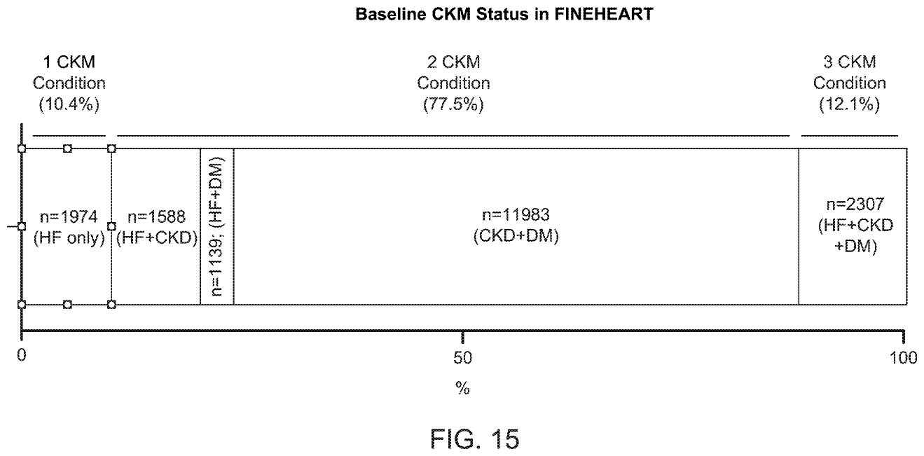

Mean age was 67±10 years, 35.1% were women, and participants were enrolled across all major geographic regions. Participants were at high risk for CKD progression (FIG. 14) with either reduced eGFR (30.1% with eGFR <45 and 26% with eGFR 45 to 60 mL/min/1.73 m²) and/or albuminuria (30.8% with “A2” UACR 30-299 mg/g and 49.2% with “A3” UACR≥300 mg/g). 2,307 (12.1%) participants had all CKM conditions (HF, CKD, and diabetes); FIG. 15. At baseline, 1,690 (8.9%) participants were co-treated with an SGLT2i and 1,110 (5.8%) with a GLP-1RA. Baseline characteristics and concurrent medical management were well-balanced between treatment arms (Table 27.1).

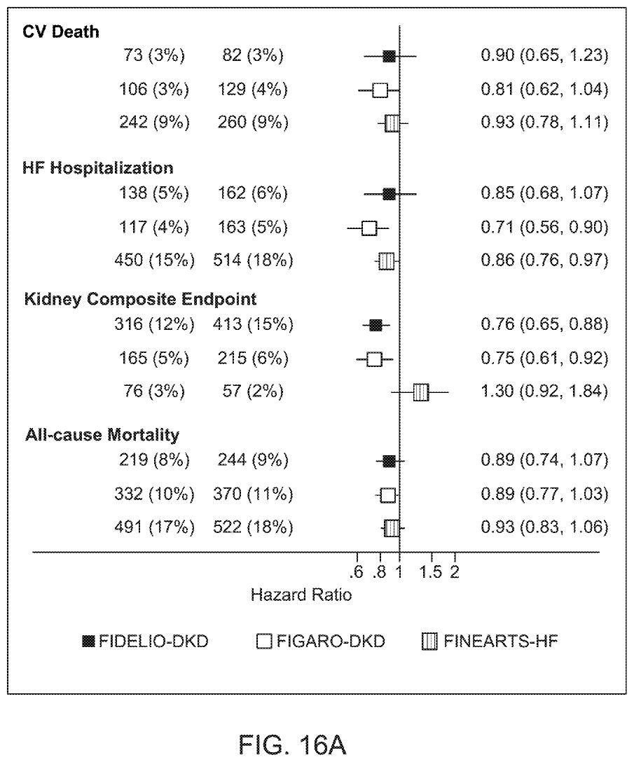

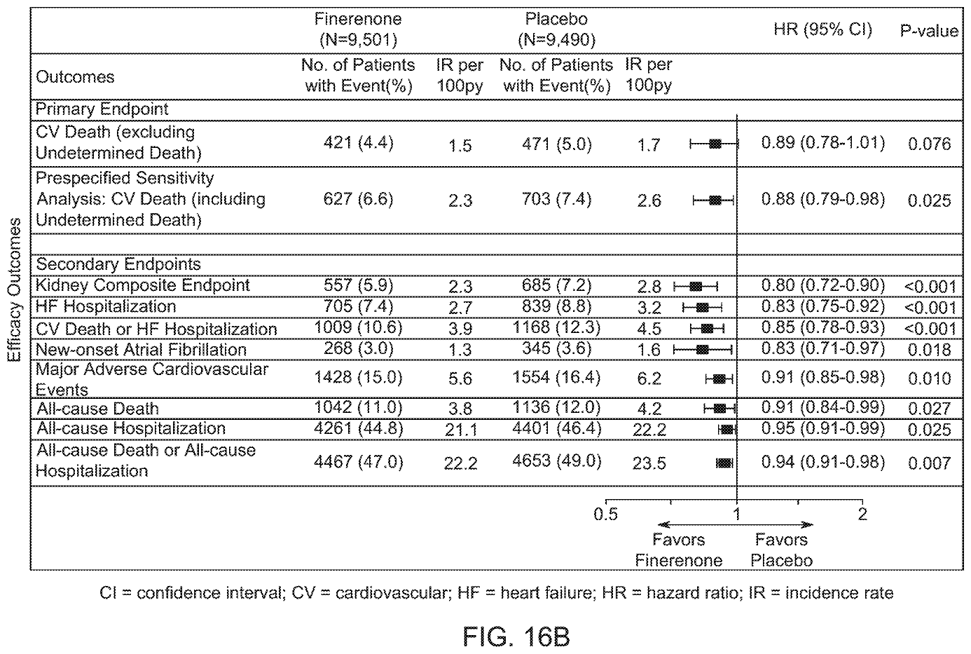

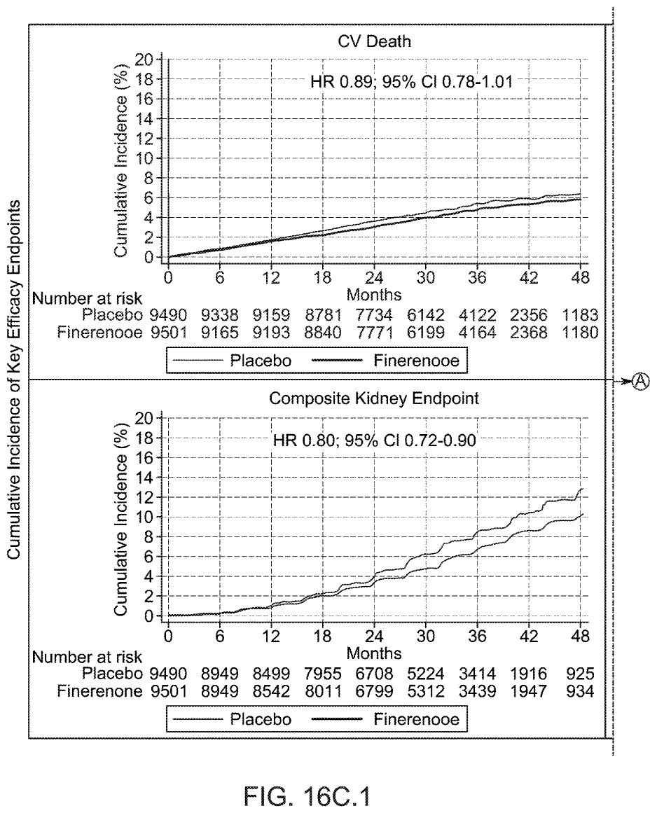

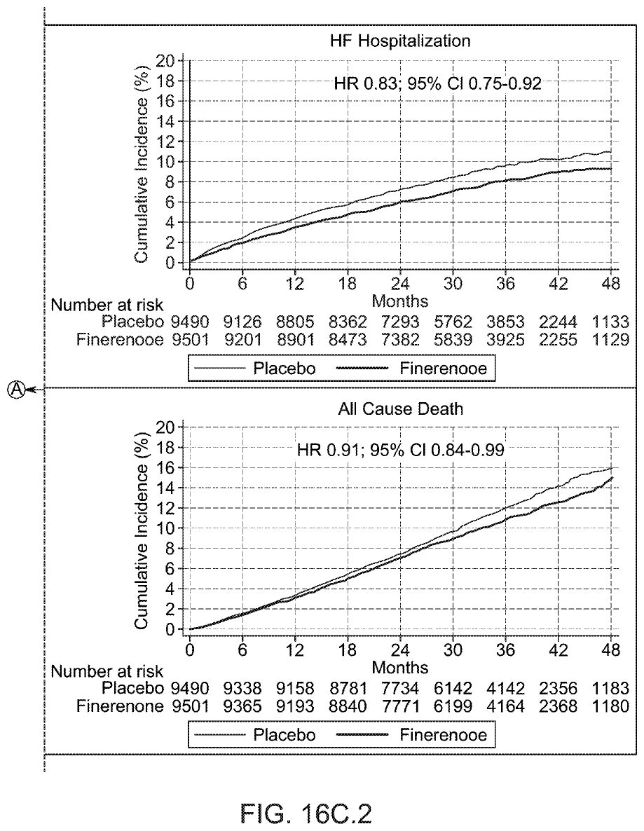

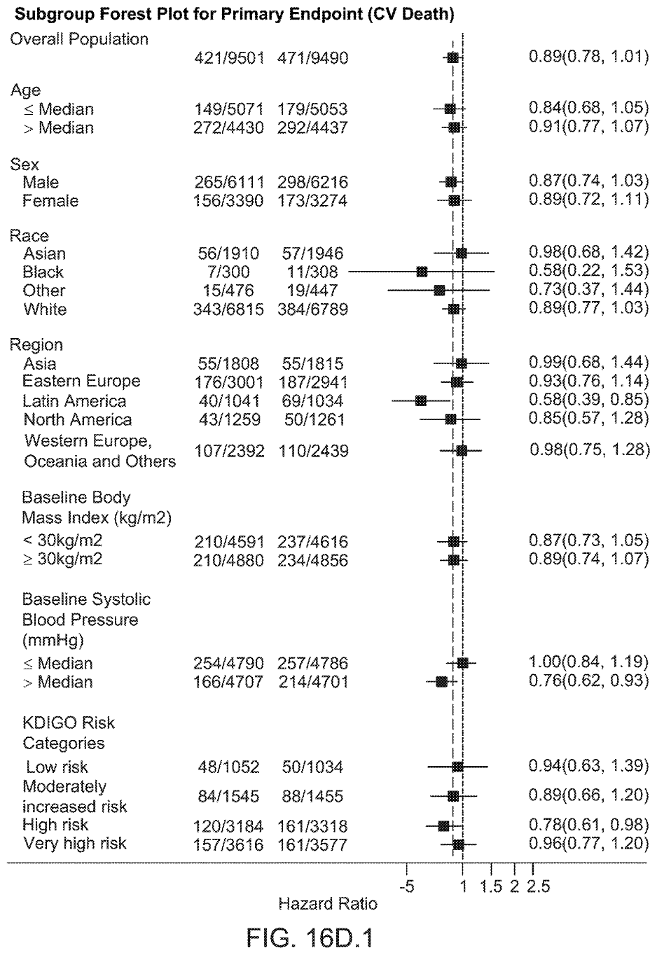

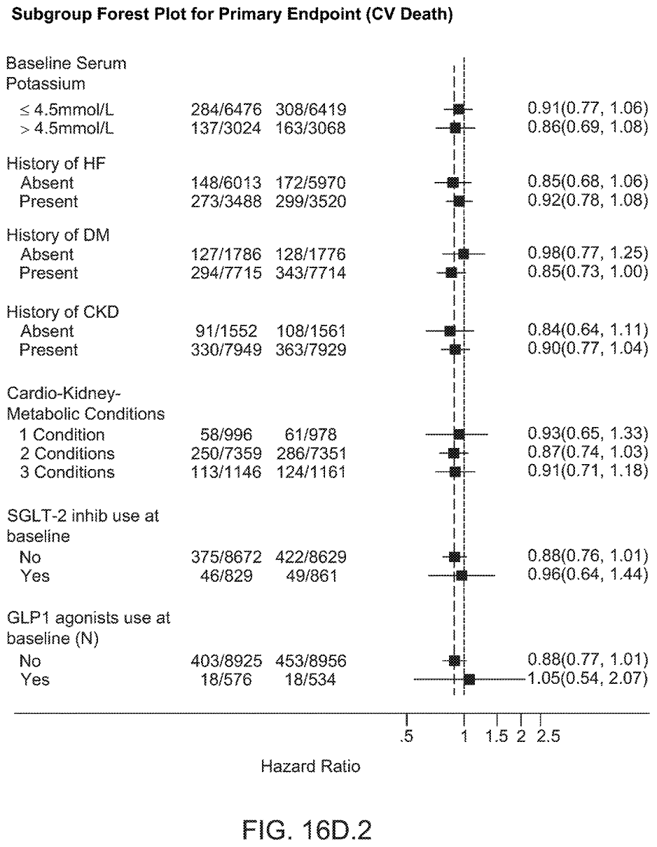

Median duration of follow-up was 2.6 years (FIDELIO-DKD), 3.4 years (FIGARO-DKD), and 2.6 years (FINEARTS-HF). Median follow-up of the pooled patient population was 2.9 years. Cardiovascular death occurred in 421 (4.4%) in the finerenone arm and 471 (5.0%) in the placebo arm (HR 0.89; 95% CI 0.78-1.01; P=0.076) with consistent findings in prespecified sensitivity analysis including both cardiovascular deaths and undetermined deaths (HR 0.88; 95% CI 0.79-0.98; P=0.025); FIG. 16A, FIG. 16B, FIG. 16C.1, FIG. 16C.2, FIG. 16D.1, FIG. 16D.2 show effects on cardiovascular death were consistent across individual trials: FIDELIO-DKD (HR 0.89; 95% CI 0.65-1.22); FIGARO-DKD (HR 0.81; 95% CI 0.62-1.04); and FINEARTS-HF (HR 0.93; 95% CI 0.78-1.11); Pinteraction=0.68; Deaths from any cause occurred in 1,042 (11.0%) participants in the finerenone arm and 1,136 (12.0%) in the placebo arm (HR 0.91; 95% CI 0.84-0.99; P=0.027).

Finerenone reduced the risk of the composite kidney endpoint by 20% (HR 0.80; 95% 0.72-0.90; P<0.001) with effects that appeared to be driven by FIDELIO-DKD and FIGARO-DKD (FIG. 16C.1). Finerenone reduced the risk of hospitalizations due to HF alone (HR 0.83; 95% CI 0.75-0.92; P<0.001) and the composite of cardiovascular death or HF hospitalization (HR 0.85; 95% CI 0.78-0.93; P<0.001). Hospitalizations of any cause were also lower with finerenone compared with placebo (HR 0.95; 95% 0.91-0.99; P=0.025). Finerenone further reduced the risk of the composite of all-cause death or all-cause hospitalization (HR 0.94; 0.91-0.98; P=0.007).

Additional risk reductions were observed for the prevention of new-onset atrial fibrillation and major adverse cardiovascular events (FIG. 16B).

Treatment effects on cardiovascular death were generally consistent across the 16 subgroups examined (FIG. 16D.1, FIG. 16D.2). The efficacy of finerenone on cardiovascular death was consistent across the range of eGFR (Pinteraction=0.32) and UACR (Pinteraction=0.55). Treatment effects on cardiovascular death were also consistent across a range of CKM disease burden: 1 condition (HR 0.93; 95% CI 0.65-0.1.33); 2 conditions (HR 0.87; 95% CI 0.74-1.03); and 3 conditions (HR 0.91; 95% CI 0.71-1.18); Pinteraction=0.94.

Incidences of any serious adverse event were lower with finerenone than placebo (34.6% vs. 36.6%), although incidences of serious adverse events leading to drug discontinuation were slightly higher with finerenone (5.4% vs. 4.6%). Laboratory-defined hyperkalemia was increased, while laboratory-defined hypokalemia was decreased with finerenone. Incidences of investigator-reported hyperkalemia leading to permanent treatment discontinuation (1.3% vs. 0.5%) and hyperkalemia-related hospitalization (0.8% vs. 0.2%) were higher with finerenone. There were no deaths related to hyperkalemia and no between-group differences in incidences of acute kidney injury (Table 27.3).

TABLE 27.3
Safety Outcomes

| | Finerenone | Placebo |
|---|---|---|
| | n = 9,482 | n = 9,467 |
| Any serious adverse event | 3,283 (34.6%) | 3,463 (36.6%) |
| Any adverse event leading to treatment discontinuation | 515 (5.4%) | 434 (4.6%) |
| Any potassium >5.5 mmol/L^{a} | 1,535 (16.5%) | 714 (7.7%) |
| Any potassium >6.0 mmol/L^{a} | 311 (3.3%) | 133 (1.4%) |
| Any potassium <3.5 mmol/L^{a} | 448 (4.8%) | 938 (10.1%) |
| Hyperkalemia^{b} | 1,216 (12.8%) | 586 (6.2%) |
| Hyperkalemia leading to treatment discontinuation^{b} | 123 (1.3%) | 43 (0.5%) |
| Hyperkalemia leading to hospitalization^{b} | 80 (0.8%) | 17 (0.2%) |
| Hyperkalemia leading to death^{b} | 0 (0.0%) | 0 (0.0%) |
| Acute kidney injury | 345 (3.6%) | 316 (3.3%) |
| Acute kidney injury leading to treatment discontinuation | 15 (0.2%) | 12 (0.1%) |
| Acute kidney injury leading to hospitalization | 143 (1.5%) | 116 (1.2%) |
| Systolic blood pressure <100 mmHg | 1,040 (11.1%) | 651 (7.0%) |
| Gynecomastia or breast hyperplasia | 16 (0.2%) | 19 (0.2%) |

^{a}Based on central laboratory measurements of potassium levels
^{b}Based on investigator reported adverse events

Treatment-emergent adverse events are defined as any adverse event occurring in any patient who has received at least one dose of study drug and within three days of permanent discontinuation. This safety table includes 1 patient who was randomized to placebo but who actually received finerenone.

### Discussion

This participant-level pooled analysis FINE-HEART represents the largest analysis of the efficacy and safety of the non-steroidal MRA finerenone across the CKM spectrum. While this pooled analysis failed to demonstrate a significant reduction in the primary endpoint of cardiovascular death, this result was sensitive to the definition of cardiovascular death based on the classification of deaths of undetermined causes. As such, we placed greater confidence in the endpoint of all-cause mortality, which was reduced with finerenone with nominal significance. This mortality signal with finerenone was further substantiated by clinically relevant benefits observed across a broad range of other cardio-kidney outcomes including kidney disease progression, HF hospitalizations, all-cause hospitalizations, new-onset atrial fibrillation, and major adverse cardiovascular events. Treatment effects were consistent across all tested clinical subgroups including those with multiple, intersecting CKM conditions. No new or unexpected safety signals were uncovered in this pooled analysis with a well-characterized modestly higher risks of hyperkalemia, but overall lower incidences of serious adverse events and no excess risk of acute kidney injury with finerenone. Taken together, these data affirm the potential of finerenone to prevent or delay morbidity and mortality in patients with CKM conditions while being safe and well tolerated.

Finerenone is approved for use in patients with CKD and type 2 diabetes with albuminuria. While several major multi-specialty guidelines strongly recommend finerenone to delay CKD progression and prevent HF events in people with CKD with type 2 diabetes. The latest KDIGO guideline has offered a class 2A recommendation potentially related to residual uncertainties regarding the mortality effects of finerenone in the FIDELIO-DKD and FIGARO-DKD trials. Furthermore, according to the guideline, its use is to be considered in patients already on standard-of-care therapies such as maximally tolerated renin-angiotensin system inhibitors and/or SGLT2 inhibitors. These pooled data did not identify heterogeneity with finerenone's effects on cardiovascular death by background use of SGLT2 inhibitors or GLP-1 receptor agonists, although the statistical power of these subgroup findings was limited. This pooled analysis bolsters recent calls for the combination use of finerenone alongside these therapies as foundational “pillars” of care to maximize improvements in cardio-kidney outcomes.

Finerenone was shown to robustly reduce the kidney composite outcome by 20% in this pooled analysis, driven by benefits observed in FIDELIO-DKD and FIGARO-DKD. FINEARTS-HF enrolled a primary heart failure population with some coexisting kidney disease, but with relatively low levels of albuminuria; therefore, CKD progression over a relatively short period of follow-up was difficult to evaluate. It is reassuring that finerenone did not show an increase in reports of acute kidney injury, despite the high baseline kidney risk profile of the patient population and the varied clinical care settings of therapeutic initiation.

While each trial had broad eligibility criteria, some groups were understudied in individual trials. For instance, both FIGARO-DKD and FIDELIO-DKD exclusively enrolled participants with CKD and type 2 diabetes with albuminuria. FINEARTS-HF provides supportive and complementary evidence related to finerenone's therapeutic effects in previously understudied populations including those without diabetes (~60% of trial enrollment), those without CKD, and those without significant urinary albumin excretion (>60% of the trial with UACR<30 mg/g). FINEARTS-HF exclusively enrolled patients with symptomatic HF across clinical care settings while FIDELIO-DKD and FIGARO-DKD specifically excluded patients with symptomatic HF with reduced ejection fraction. These pooled data demonstrate that finerenone's benefits extend to patients with different degrees of CKM multimorbidity and across broad patient profiles. The diverse spectrum of benefits on HF, arrhythmia, atherosclerotic risk, and kidney disease progression also underscores the systemic actions of finerenone in attenuating the adverse multi-organ effects of MR overactivation.

Study Strengths and Limitations. A key strength of this pooled analysis was access to individual participant-level data from all phase III trials conducted to date with finerenone, which allowed harmonization of data elements related to baseline clinical characteristics, outcomes, and subgroups. Major efficacy outcomes were independently adjudicated and safety assessments were conducted with aligned definitions in a standardized fashion. It is noteworthy that the findings of this pooled data analysis were derived from randomized clinical trials with specific inclusion and exclusion criteria and thus may not be generalizable to all populations treated in clinical practice. Despite the large global population studied, enrollment of select groups such as Black patients remained limited. Certain data elements were not consistently available across trials to allow for pooling. For instance, urgent HF visits, which were included as a part of the FINEARTS-HF primary endpoint, were not collected in FIDELIO-DKD and FIGARO-DKD.

While this pooled analysis failed to demonstrate significant reductions in cardiovascular death, finerenone was associated with significantly lower deaths of any cause, cardiovascular events, and kidney outcomes, while being safe and well-tolerated. The totality of the evidence thus supports the disease-modifying potential of finerenone in broad, high-risk patient populations encompassing cardiovascular, kidney, and metabolic diseases.

Finerenone was associated with a non-significant reduction in CV death not including unknown death; however, this became significant when unknown death was included. The effects of finerenone were consistent regardless of baseline medication use and level of CKM comorbidity (i.e., HF, T2D and CKD). Finerenone demonstrated significant benefits in other prespecified outcomes, including kidney disease progression, all-cause death and HHF.

Finerenone was well tolerated, with a well-characterized modestly higher risk of hyperkalemia versus placebo, but overall lower incidence of serious adverse events and no excess risk of acute kidney injury. This analysis strengthens recent support for the combination use of finerenone alongside SGLT-2 is and GLP-1RAs as foundational ‘pillars’ of care to maximize improvements in cardio-kidney outcomes.

### Example 15—Metaanalysis-RALES/EMPHASIS-HF/TOPCAT/FINEARTS-HF

Mineralocorticoid receptor antagonists (MRAs) reduce hospitalizations and death in patients with heart failure and reduced ejection fraction (HFrEF) but the benefit in patients with HF and mildly reduced or preserved EF (HFmrEF/HFpEF) is unclear. The effect of MRAs in four trials across the range of EF was examined.

This is a pre-specified, individual patient-level meta-analysis including RALES (spironolactone) and EMPHASIS-HF (eplerenone), enrolling HFrEF patients, and TOPCAT (spironolactone) and FINEARTS-HF (finerenone), enrolling HFmrEF/HFpEF patients. The effect of MRAs was estimated on the outcomes of cardiovascular death or HF hospitalization, components of this composite, total HF hospitalizations (with and without cardiovascular deaths) and all-cause death. An interaction between trials and treatment was tested to examine the heterogeneity of effect in these populations. The study is registered with PROSPERO, CRD42024541487.

In 13,846 patients, MRAs reduced the risk of cardiovascular death or HF hospitalization (hazard ratio [HR] 0.77 [95% CI 0.72-0.83]). There was a statistically significant interaction by trials and treatment, p for interaction<0.001 due to the greater efficacy in HFrEF (HR 0.66 [95% CI 0.59-0.73]) compared to HFmrEF/HFpEF (HR 0.87 [0.79-0.95]). We observed significant reductions in HF hospitalisation in the HFrEF trials (HR 0.63 [0.56-0.72]) and the HFmrEF/HFpEF trials (HR 0.82 [0.74-0.91]). The same pattern was observed for total HF hospitalizations with or without cardiovascular death. Cardiovascular death was reduced in the HFrEF trials (HR 0.72 [0.63-0.82]) but not the HFmrEF/HFpEF trials (HR 0.92 [0.80-1.05]). With an MRA, the risk of hyperkalemia was doubled compared to placebo, but the incidence of serious hyperkalemia (potassium >6.0 mmol/L) was low (2.9% versus 1.4%); the risk of hypokalaemia (potassium <3.5 mmol/L) was halved (7% versus 14%).

We performed an individual patient-level meta-analysis of all four of the large prospective placebo-controlled trials of MRAs in heart failure. This analysis included nearly 14,000 patients and confirms the large benefit in patients with heart failure and reduced ejection fraction but also shows that MRAs reduce the risk of the composite of cardiovascular death or hospitalization for heart failure in patients with heart failure and an ejection fraction ≥40%. The benefits were consistent across a broad range of subgroups.

Two pivotal clinical trials, RALES (Randomized Aldactone Evaluation Study) and EMPHASIS-HF (Eplerenone in Mild Patients Hospitalization and Survival Study in Heart Failure) demonstrated that the steroidal mineralocorticoid receptor antagonists (MRAs), spironolactone and eplerenone decrease the risk of death and hospitalization in patients with heart failure and reduced ejection fraction (HFrEF). As a result, international guidelines make consistent and strong recommendations for spironolactone and eplerenone in patients with HFrEF. By contrast, the efficacy of these agents in heart failure with mildly reduced or preserved ejection (HFmrEF/HFpEF) is uncertain. Specifically, spironolactone did not improve the primary outcome of first heart failure hospitalization or cardiovascular death in the TOPCAT trial (Treatment of Preserved Cardiac Function Heart Failure with an Aldosterone Antagonist). However, a significant proportion of participants in that trial may not have had heart failure or taken randomized treatment, and post hoc analyses suggested possible benefit in those who did. Consequently, guideline recommendations for MRAs in heart failure with HFmrEF/HFpEF are weak or absent. The question of whether the non-steroidal MRA finerenone is efficacious in patients with HFmrEF/HFpEF was recently evaluated in the FINEARTS-HF trial (the finerenone trial to investigate efficacy and safety superior to placebo in patients with heart failure). Unlike spironolactone and eplerenone, finerenone is a non-steroidal MRA, a class with different physiochemical properties, and in FINEARTS-HF finerenone significantly reduced the risk of the primary composite outcome of total worsening heart failure events and cardiovascular death in patients with heart failure and an ejection fraction of 40% or above. We undertook a pre-specified individual patient-level meta-analysis of the four MRA trials to test the consistency of the effects of mineralocorticoid receptor antagonism across important clinical outcomes, including endpoints that no single trial was designed or powered to examine such as cardiovascular mortality, and across both MRA classes. Furthermore, we examined key safety outcomes and the efficacy and safety of treatment over a range of clinically important subpopulations including those at the highest end of the ejection fraction range and with kidney dysfunction.

### Methods

### Search Strategy and Selection Criteria

Our pre-specified aim was to study the efficacy and safety of MRAs across the full range of ejection fraction in patients with heart failure and according to MRA class. We analyzed the RALES trial with spironolactone in patients with HF and left ventricular ejection fraction (LVEF) £ 35%, EMPHASIS-HF with eplerenone in patients with HF and LVEF £ 35% (if LVEF >30 to 35%, a QRS duration of >130 msec on electrocardiogramas required), TOPCAT with spironolactone in patients with HF and LVEF 3 45% and FINEARTS-HF with finerenone in patients with HF and LVEF 3 40%. Key information on the included trials and their inclusion and exclusion criteria along with the primary outcomes of each of the trials is given. To ensure that we did not exclude any other important trials we conducted a systematic review of MEDLINE via PubMed of randomized trials of MRAs in patients with HF. Trials were included if they included at least 1000 patients with heart failure with an appropriately powered morbidity or mortality outcome. Data from the FINEARTS-HF trial were unpublished at the time of analysis, but were included for the current Example. Data were extracted, harmonized, and analyzed.

### Outcomes and Subgroups

The primary outcome of interest in this meta-analysis was a composite of time to first hospitalization for HF or cardiovascular death. Secondary outcomes examined were time-to-first hospitalization for heart failure, total (first and repeat) heart failure hospitalizations, total heart failure hospitalization and cardiovascular death, cardiovascular death, and all-cause death. In each trial, outcomes were adjudicated by a blinded clinical endpoints committee. Cardiovascular death was analyzed according to the original definition reported in each of the trials. Because the definition of cardiovascular death included undetermined deaths in RALES, EMPHASIS-HF and TOPCAT but not FINEARTS-HF, we conducted a sensitivity analysis with and without undetermined deaths counted as cardiovascular deaths. Unlike the older trials, FINEARTS-HF included urgent visits for worsening heart failure in the primary composite outcome as an equivalent to heart failure hospitalization, reflecting changing clinical practice aimed at reducing admissions (and a practice which may have been more common during the COVID-19 pandemic). These events were defined as urgent, unscheduled ambulatory, or emergency room visits for the primary diagnosis of heart failure requiring intravenous diuretic or a vasoactive agent or mechanical or surgical intervention for heart failure. Intensification of an oral diuretic alone was not sufficient to meet this definition.

The effect of MRAs on the primary outcome was examined in key subgroups of interest, including age, sex, race, geographical region, body mass index (BMI), New York Heart Association (NYHA) class, LVEF, history of prior hospitalization for heart failure, creatinine, estimated glomerular filtration rate, potassium, systolic blood pressure, history of diabetes, myocardial infarction, atrial fibrillation, stroke, and treatment with an ACE inhibitor or angiotensin receptor blocker, beta blocker, diuretic or a digitalis glycoside. A further subgroup of interest was the efficacy of steroidal (spironolactone and eplerenone) versus non-steroidal MRAs (finerenone).

Safety outcomes were examined in patients who received at least one dose of randomized treatment. The outcomes of interest included serum creatinine ≥2.5 and ≥3 mg/dL, a >20% and >30% decline in eGFR, serum potassium <3.5 mmol/L, serum potassium >5.5 and >6 mmol/L and systolic blood pressure <90 and <100 mmHg.

### Statistical Analysis

For all time-to-first event outcomes, point estimates from Cox proportional hazards models are presented as hazard ratios (HRs) with 95% CIs on an intention-to-treat basis. These models included a term for randomized treatment and were stratified by trial. Event rates for time to first events were calculated per 100 person-years and estimated using the normal approximation to the Poisson log-likelihood. For analysis of total events, rate ratios (RRs) with 95% CIs are presented from a negative binomial model as the time to subsequent hospitalizations was not available in RALES and the results were confirmed in a semi-parametric proportional rates model with a factor for randomized treatment and stratified by trial excluding RALES but including the other trials. Recurrent event rates were estimated from a Poisson model with robust standard errors. Between-trial heterogeneity of treatment effect was examined in the models described with an interaction term between trial and randomized therapy. Additionally, we tested treatment-by-trial heterogeneity of effect using Cochran's Q test and Higgins and Thompsons' I² from a two-stage meta-analysis using a random effects model. Subgroup analysis was performed in time-to-event Cox models with an interaction term between the subgroup of interest and randomized therapy in the model stratified by trial. For continuous variables, a restricted cubic spline was used with knots placed at points that resulted in the model with the lowest Akaike's information criterion value. An interaction term between the spline and randomized therapy was tested in the model and the interaction was represented graphically. The effect of randomized therapy on safety outcomes was assessed by calculating an odds ratio in a logistic regression model with a factor for treatment and trial and the interaction tested with a treatment by trial interaction term. A p-value below 0.05 was considered statistically significant.

The protocol and statistical analysis plan for the meta-analysis were pre-specified before the FINEARTS-HF trial database was locked and were pre-registered on PROSPERO (CRD42024541487). All trials were assessed as high quality but the risk of bias for TOPCAT with regards to the assessment of deviation from the intended intervention was high. A sensitivity analysis only including the participants from the Americas was therefore undertaken to examine the impact of this potential bias. All participants provided written consent, and the study protocols were approved by the institutional review boards at all participating sites. All analyses were performed using Stata (version 18.0).

### Results

Overall, 13,846 patients were included in the four trials. Patients enrolled in RALES and EMPHASIS-HF were more often men and were less likely to have a history of hypertension, atrial fibrillation, or diabetes than those enrolled in TOPCAT and FINEARTS-HF (Table 28).

TABLE 28
Baseline characteristics of patients in each mineralocorticoid
receptor antagonist trial and in the total population studied

| | RALES | EMPHASIS-HF | TOPCAT | FINEARTS-HF | Total |
|---|---|---|---|---|---|
| | N = 1,663 | N = 2,737 | N = 3,445 | N = 6,001 | N = 13,846 |
| Age (years) | 65 ± 11 | 68 ± 7 | 68 ± 9 | 72 ± 9 | 69 ± 9 |
| Sex | | | | | |
| Men | 1,217 (73) | 2,127 (78) | 1,670 (48) | 3,269 (54) | 8,283 (60) |
| Women | 446 (27) | 610 (22) | 1,775 (52) | 2,732 (46) | 5,563 (40) |
| Race | | | | | |
| White | 1,440 (87) | 2,268 (83) | 3,062 (89) | 4,735 (79) | 11,505 (83) |
| Black | 120 (7) | 67 (2) | 302 (9) | 88 (1) | 577 (4) |
| Asian | 32 (2) | 316 (12) | 19 (1) | 996 (17) | 1,363 (10) |
| Other | 71 (4) | 86 (3) | 62 (2) | 182 (3) | 401 (3) |
| Region | | | | | |
| North America | 114 (7) | 248 (9) | 1,477 (43) | 471 (8) | 2,310 (17) |
| Latin America | 433 (26) | 98 (4) | 290 (8) | 641 (11) | 1,462 (11) |
| Western Europe | 1,066 (64) | 1,005 (37) | 0 (0) | 1,204 (20) | 3,275 (24) |
| Central and | 0 (0) | 988 (36) | 1,678 (49) | 2,630 (44) | 5,296 (38) |
| Eastern Europe | | | | | |
| Asia-Pacific | 50 (3) | 398 (15) | 0 (0) | 1,055 (18) | 1,503 (11) |
| BMI (kg/m²) | | 27.5 ± 4.9 | 32.1 ± 7.1 | 29.9 ± 6.1 | 30.0 ± 6.4 |
| <30 kg/m² | * | 1,983 (73) | 1,533 (45) | 3,296 (55) | 6,812 (56) |
| ≥30 kg/m² | * | 739 (27) | 1,902 (55) | 2,692 (45) | 5,333 (44) |
| Systolic blood | 122 ± 20 | 124 ± 17 | 129 ± 14 | 129 ± 15 | 127 ± 16 |
| pressure (mmHg) | | | | | |
| Heart rate | 81 ± 14 | 72 ± 13 | 69 ± 10 | 71 ± 11 | 72 ± 12 |
| (beats/min) | | | | | |
| LVEF (%) | 25 ± 7 | 26 ± 5 | 57 ± 7 | 53 ± 8 | 45 ± 15 |
| NYHA class, N (%) | | | | | |
| I, II | 7 (0) | 2,730 (100) | 2,303 (67) | 4,146 (69) | 9,186 (66) |
| III, IV | 1,656 (100) | 3 (0) | 1,136 (33) | 1,854 (31) | 4,649 (34) |
| Prior HF | * | 1,438 (53) | 2,489 (72) | 3,619 (60) | 7,546 (62) |
| hospitalization, | | | | | |
| N (%) | | | | | |
| NT-proBNP | ** | ** | 843.0 | 1,041.4 | 1,013.5 |
| (pg/ml) | | | (463.0- | (448.5- | (449.6- |
| | | | 1720.0) | 1945.9) | 1929.8) |
| eGFR (ml per | 63 ± 22 | 65 ± 18 | 65 ± 19 | 63 ± 20 | 64 ± 19 |
| min per 1.73 m²) | | | | | |
| eGFR | | | | | |
| (ml/min/1.73 m²) | | | | | |
| <60 | 841 (51) | 1,092 (40) | 1,463 (42) | 2,844 (47) | 6,240 (45) |
| >=60 | 817 (49) | 1,633 (60) | 1,980 (58) | 3,157 (53) | 7,587 (55) |
| Potassium | 4.2 ± 0.4 | 4.3 ± 0.4 | 4.3 ± 0.4 | 4.4 ± 0.5 | 4.3 ± 0.5 |
| (mmol/L) | | | | | |
| Diabetes, N (%) | 369 (22) | 859 (31) | 1,118 (32) | 2,439 (41) | 4,785 (35) |
| Hypertension, N (%) | 391 (24) | 1,819 (66) | 3,147 (91) | 5,325 (89) | 10,682 (77) |
| Atrial | 183 (11) | 844 (31) | 1,214 (35) | 3,273 (55) | 5,514 (40) |
| fibrillation, N (%) | | | | | |
| Myocardial | 472 (28) | 1,380 (50) | 893 (26) | 1,541 (26) | 4,286 (31) |
| infarction, N (%) | | | | | |
| Stroke, N (%) | * | 262 (10) | 265 (8) | 708 (12) | 1,235 (10) |
| ACEI/ARB, N (%) | 1,589 (96) | 2,558 (94) | 2,900 (84) | 4,246 (71) | 11,293 (82) |
| β-Blocker, N (%) | 171 (10) | 2,374 (87) | 2,676 (78) | 5,095 (85) | 10,316 (75) |
| Diuretic, N (%) | 1,502 (90) | 2,326 (85) | 2,817 (82) | 5,930 (99) | 12,575 (91) |
| Digitalis | 1,216 (73) | 740 (27) | 342 (10) | 471 (8) | 2,769 (20) |
| glycosides, N (%) | | | | | |

NT-proBNP, N-terminal pro B type natriuretic peptide BMI, body mass index eGFR, estimated glomerular filtration rate ACEI, angiotensin-converting enzyme inhibitor ARB, angiotensin receptor blocker LVEF, left ventricular ejection fraction NYHA, New York Heart Association
* missing in RALES
** missing in RALES and EMPHASIS-HF

The estimated glomerular filtration rate at baseline was lowest in RALES and FINEARTS-HF. Baseline characteristics by randomized therapy are given below in Table 29 and were balanced between treatment groups.

TABLE 29
Baseline characteristics in the included trials by randomized therapy

| | RALES | RALES | EMPHASIS-HF | EMPHASIS-HF | TOPCAT | TOPCAT | FINEARTS-HF | FINEARTS-HF |
|---|---|---|---|---|---|---|---|---|
| | spironolactone | placebo | eplerenone | placebo | spironolactone | placebo | finerenone | placebo |
| | N = 822 | N = 841 | N = 1,364 | N = 1,373 | N = 1,722 | N = 1,723 | N = 3,003 | N = 2,998 |
| Age (years) | 65 ± 12 | 65 ± 11 | 68 ± 7 | 68 ± 7 | 68 ± 9 | 68 ± 9 | 71 ± 9 | 72 ± 9 |
| Sex | | | | | | | | |
| Male | 603 | 614 | 1,055 | 1,072 | 834 | 836 | 1,648 | 1,621 |
| | (73) | (73) | (77) | (78) | (48) | (49) | (55) | (54) |
| Female | 219 | 227 | 309 | 301 | 888 | 887 | 1,355 | 1,377 |
| | (27) | (27) | (23) | (22) | (52) | (51) | (45) | (46) |
| Race | | | | | | | | |
| White | 712 | 728 | 1,127 | 1,141 | 1,525 | 1,537 | 2,366 | 2,369 |
| | (87) | (87) | (83) | (83) | (89) | (89) | (79) | (79) |
| Black | 56 | 64 | 37 | 30 | 153 | 149 | 49 | 39 |
| | (7) | (8) | (3) | (2) | (9) | (9) | (2) | (1) |
| Asian | 15 | 17 | 158 | 158 | 10 | 9 | 497 | 499 |
| | (2) | (2) | (12) | (12) | (1) | (1) | (17) | (17) |
| Other | 39 | 32 | 42 | 44 | 34 | 28 | 91 | 91 |
| | (5) | (4) | (3) | (3) | (2) | ( 2) | (3) | (3) |
| Region | | | | | | | | |
| North | 56 | 58 | 126 | 122 | 738 | 739 | 235 | 236 |
| America | (7) | (7) | (9) | (9) | (43) | (43) | (8) | (8) |
| Latin | 216 | 217 | 47 | 51 | 148 | 142 | 322 | 319 |
| America | (26) | (26) | (3) | (4) | (9) | (8) | (11) | (11) |
| Western | 526 | 540 | 500 | 505 | 0 | 0 | 599 | 605 |
| Europe | (64) | (64) | (37) | (37) | (0) | (0) | (20) | (20) |
| Central | 0 | 0 | 492 | 496 | 836 | 842 | 1,319 | 1,311 |
| Europe | (0) | (0) | (36) | (36) | (49) | (49) | (44) | (44) |
| Asia- | 24 | 26 | 199 | 199 | 0 | 0 | 528 | 527 |
| Pacific | (3) | (3) | (15) | (14) | (0) | (0) | (18) | (18) |
| BMI | | | 27.5 ± 4.9 | 27.5 ± 4.9 | 32.1 ± 7.1 | 32.1 ± 7.1 | 29.9 ± 6.1 | 30.0 ± 6.1 |
| (kg/m2) | | | | | | | | |
| <30 kg/m2 | | | 975 | 1,008 | 764 | 769 | 1,658 | 1,638 |
| | | | (72) | (74) | (44) | (45) | (55) | (55) |
| ≥30 kg/m2 | | | 383 | 356 | 956 | 946 | 1,338 | 1,354 |
| | | | (28) | (26) | (56) | (55) | (45) | (45) |
| Systolic | 123 ± 21 | 122 ± 20 | 124 ± 17 | 124 ± 17 | 129 ± 14 | 129 ± 14 | 130 ± 15 | 129 ± 15 |
| blood pressure | | | | | | | | |
| (mmHg) | | | | | | | | |
| Heart rate | 81 ± 14 | 81 ± 15 | 72 ± 12 | 72 ± 13 | 69 ± 11 | 69 ± 10 | 72 ± 12 | 71 ± 12 |
| (beats/min) | | | | | | | | |
| LVEF (%) | 26 ± 7 | 25 ± 7 | 26 ± 5 | 26 ± 5 | 57 ± 7 | 57 ± 8 | 53 ± 7 | 53 ± 8 |
| NYHA, N (%) | | | | | | | | |
| I, II | 4 | 3 | 1,358 | 1,372 | 1,146 | 1,157 | 2,081 | 2,065 |
| | (0) | (0) | (100) | (100) | (67) | (67) | (69) | (69) |
| III, IV | 818 | 838 | 2 | 1 | 575 | 561 | 921 | 933 |
| | (100) | (100) | (0) | (0) | (33) | (33) | (31) | (31) |
| Prior HF | | | 714 | 724 | 1,246 | 1,243 | 1,797 | 1,822 |
| hospitalization, | | | (52) | (53) | (72) | (72) | (60) | (61) |
| N (%) | | | | | | | | |
| NT-proBNP | | | | | 826.0 | 879.5 | 1052.5 | 1028.0 |
| (pg/ml) | | | | | (460.0-1572.0) | (465.5-2033.0) | (467.3-1937.0) | (433.2-1963.1) |
| eGFR (ml | 63 ± 22 | 62 ± 21 | 66 ± 18 | 65 ± 18 | 65 ± 19 | 65 ± 18 | 63 ± 19 | 63 ± 20 |
| per min per | | | | | | | | |
| 1.73 m²) | | | | | | | | |
| eGFR | | | | | | | | |
| (ml/min/1.73 | | | | | | | | |
| m²) < 60 | | | | | | | | |
| <60 | 419 | 422 | 531 | 561 | 747 | 716 | 1,432 | 1,412 |
| | (51) | (50) | (39) | (41) | (43) | (42) | (48) | (47) |
| >=60 | 401 | 416 | 829 | 804 | 975 | 1,005 | 1,571 | 1,586 |
| | (49) | (50) | (61) | (59) | (57) | (58) | (52) | (53) |
| Potassium | 4.2 ± 0.5 | 4.2 ± 0.4 | 4.3 ± 0.4 | 4.3 ± 0.4 | 4.2 ± 0.4 | 4.3 ± 0.4 | 4.4 ± 0.5 | 4.4 ± 0.5 |
| (mmol/L) | | | | | | | | |
| Type 2 | 174 | 195 | 459 | 400 | 565 | 553 | 1,217 | 1,222 |
| diabetes | (21) | (23) | (34) | (29) | (33) | (32) | (41) | (41) |
| Hypertension, | 193 | 198 | 910 | 909 | 1,567 | 1,580 | 2,640 | 2,685 |
| N (%) | (23) | (24) | (67) | (66) | (91) | (92) | (88) | (90) |
| Atrial | 98 | 85 | 409 | 435 | 611 | 603 | 1,648 | 1,625 |
| fibrillation, | (12) | (10) | (30) | (32) | (35) | (35) | (55) | (54) |
| N (%) | | | | | | | | |
| Myocardial | 229 | 243 | 686 | 694 | 444 | 449 | 784 | 757 |
| infarction, | (28) | (29) | (50) | (51) | (26) | (26) | (26) | (25) |
| N (%) | | | | | | | | |
| Stroke, | | | 136 | 126 | 128 | 137 | 355 | 353 |
| N (%) | | | (10) | (9) | (7) | (8) | (12) | (12) |
| ACEI/ARB, | 788 | 801 | 1,283 | 1,275 | 1,452 | 1,448 | 2,124 | 2,122 |
| N (%) | (96) | (95) | (95) | (93) | (84) | (84) | (71) | (71) |
| β-Blocker, | 85 | 86 | 1,181 | 1,193 | 1,346 | 1,330 | 2,541 | 2,554 |
| N (%) | (10) | (10) | (87) | (87) | (78) | (77) | (85) | (85) |
| Diuretic, | 742 | 760 | 1,150 | 1,176 | 1,401 | 1,416 | 2,973 | 2,957 |
| N (%) | (90) | (90) | (85) | (86) | (81) | (82) | (99) | (99) |
| Digitalis | 611 | 605 | 363 | 377 | 178 | 164 | 241 | 230 |
| glycosides, | (74) | (72) | (27) | (28) | (10) | (10) | (8) | (8) |
| N (%) | | | | | | | | |

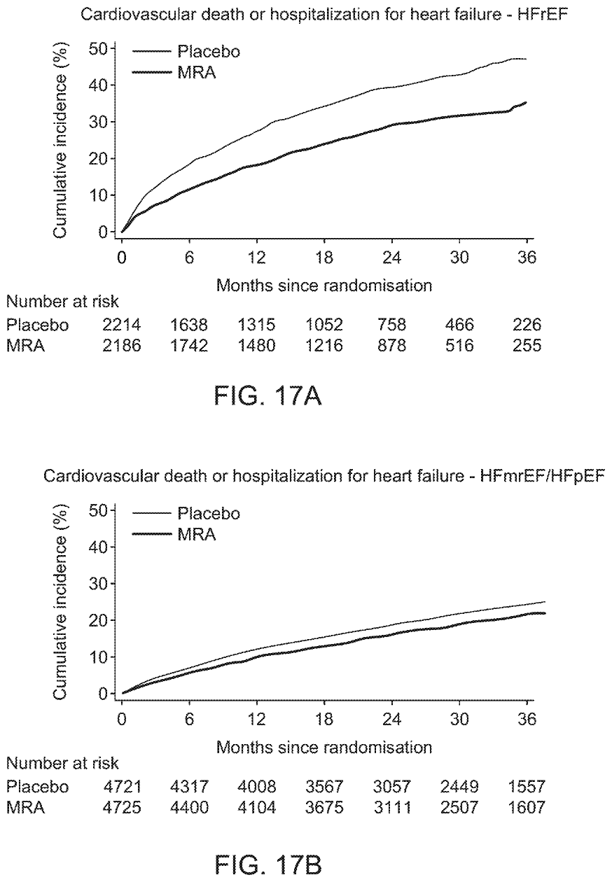

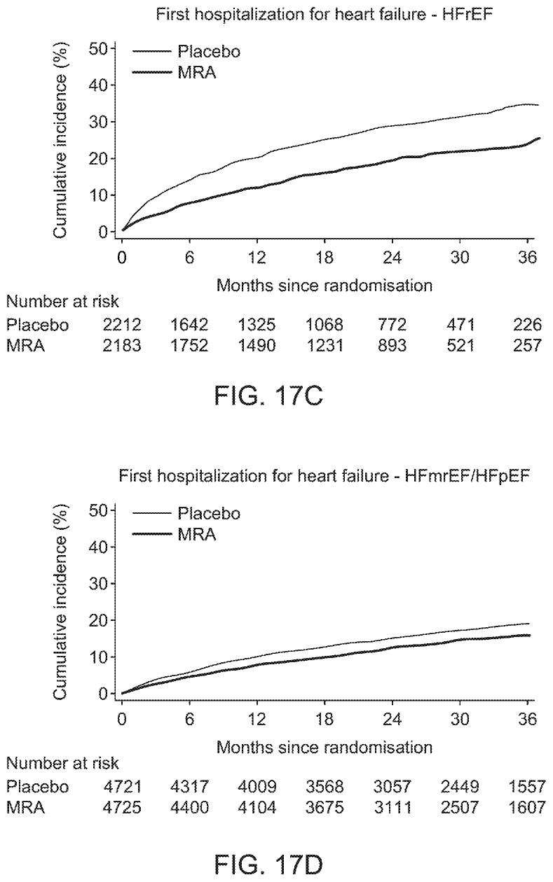

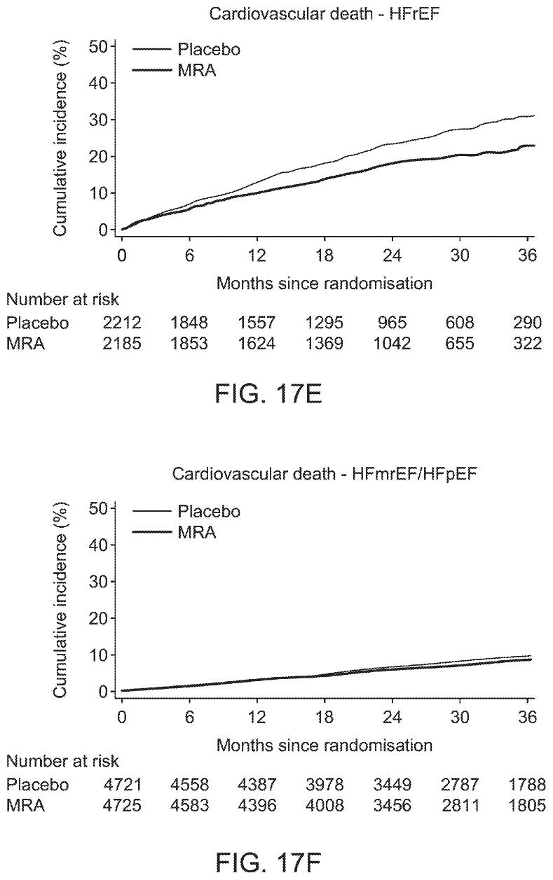

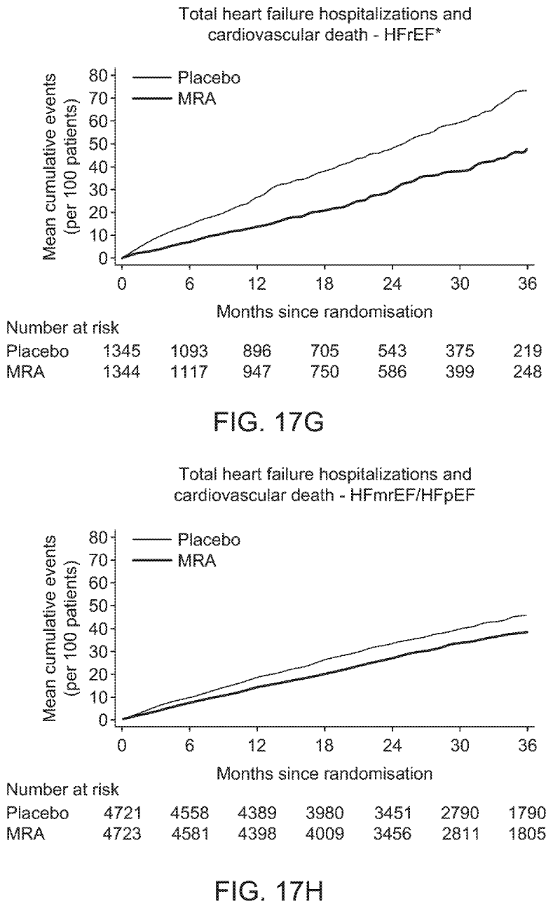

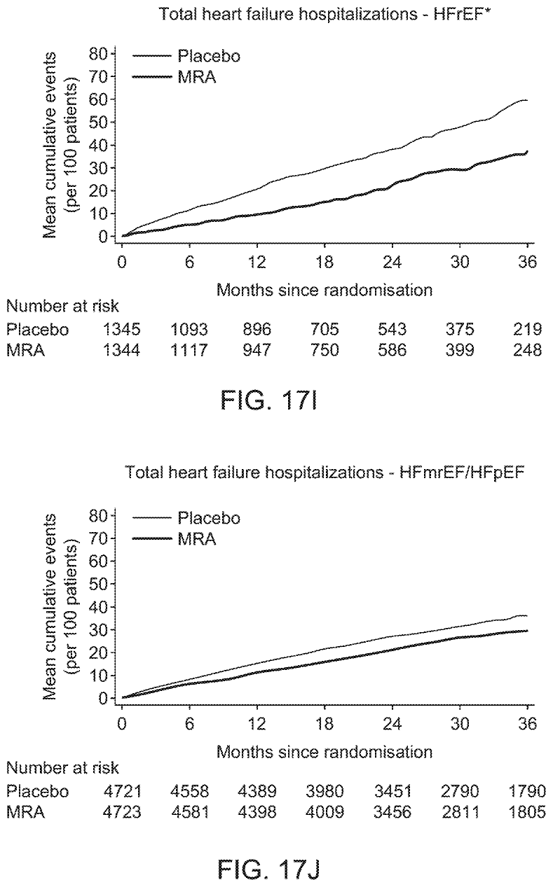

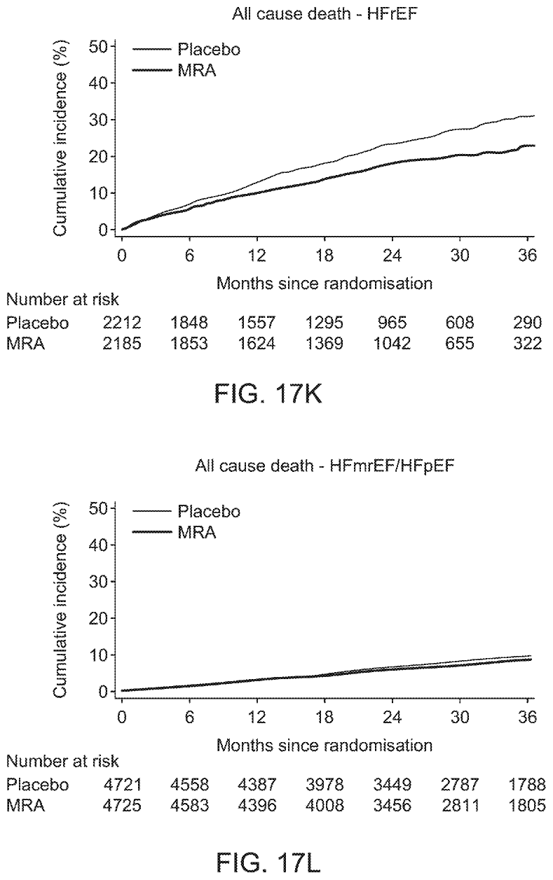

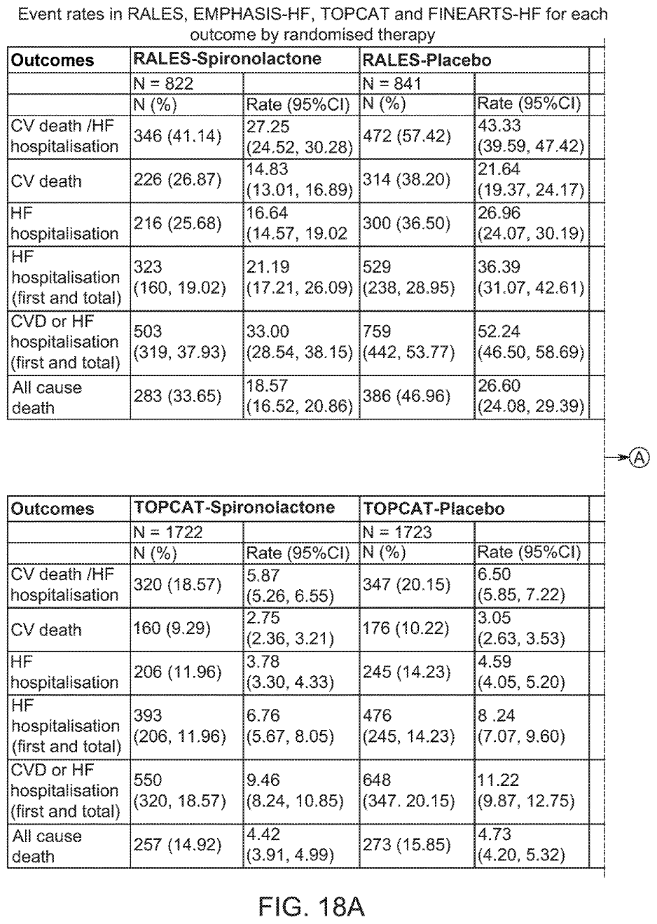

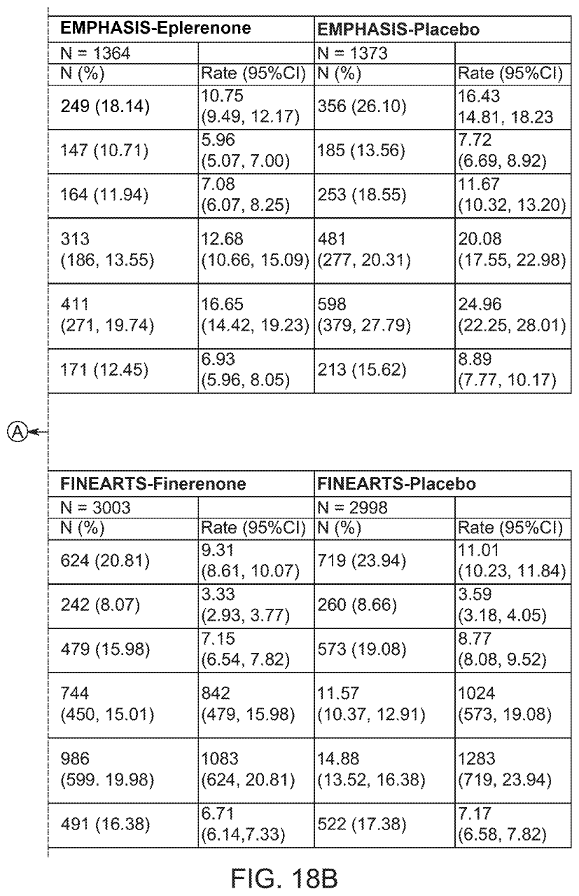

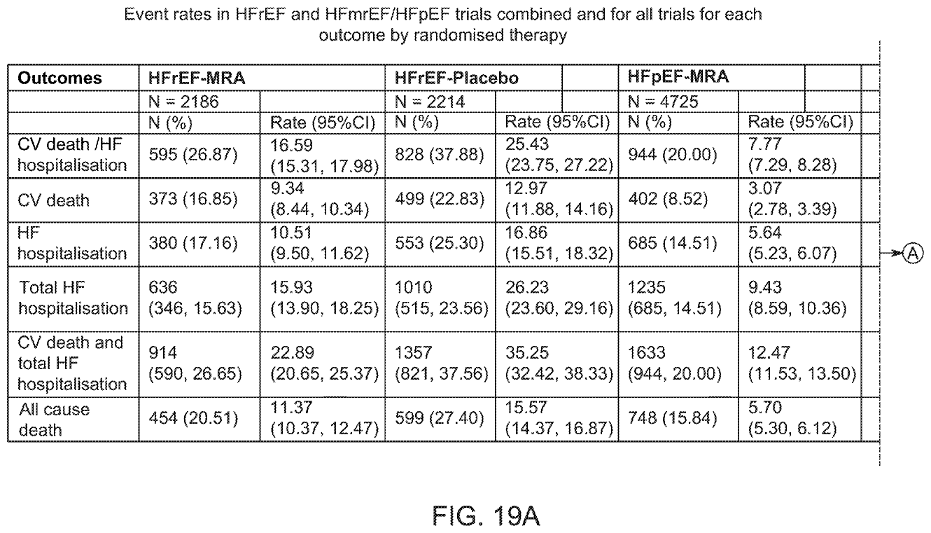

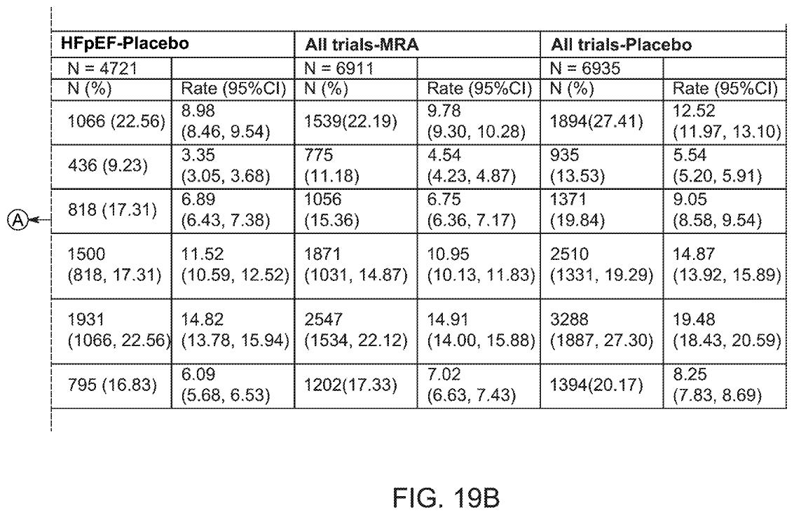

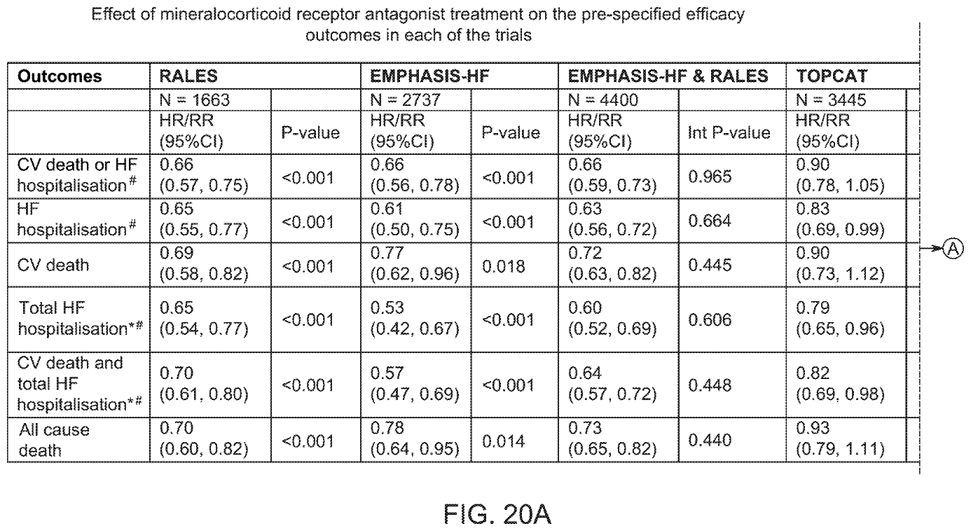

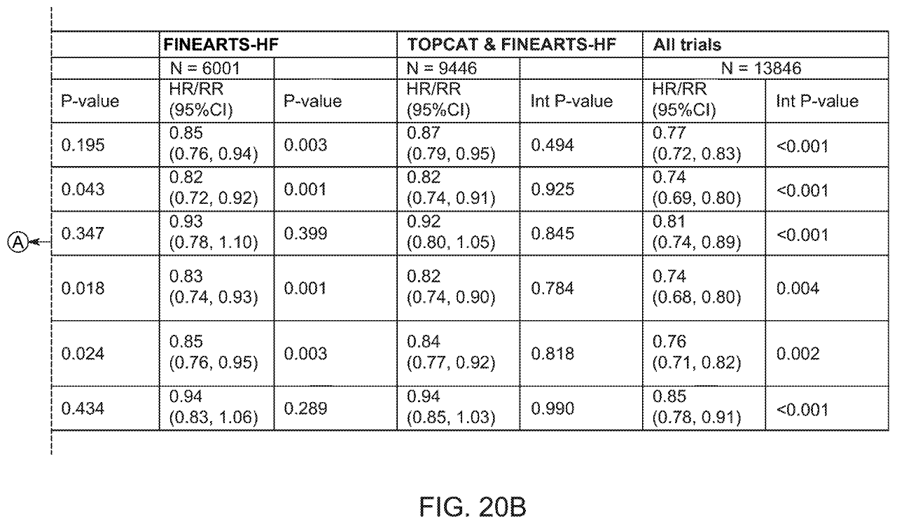

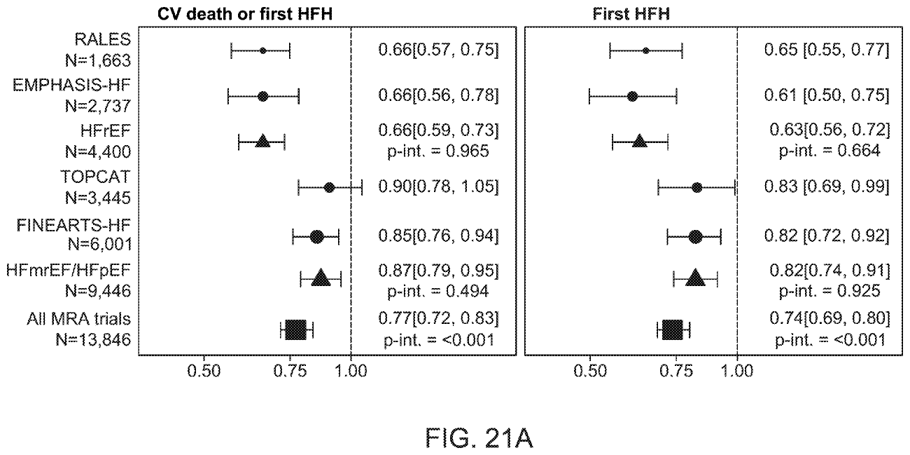

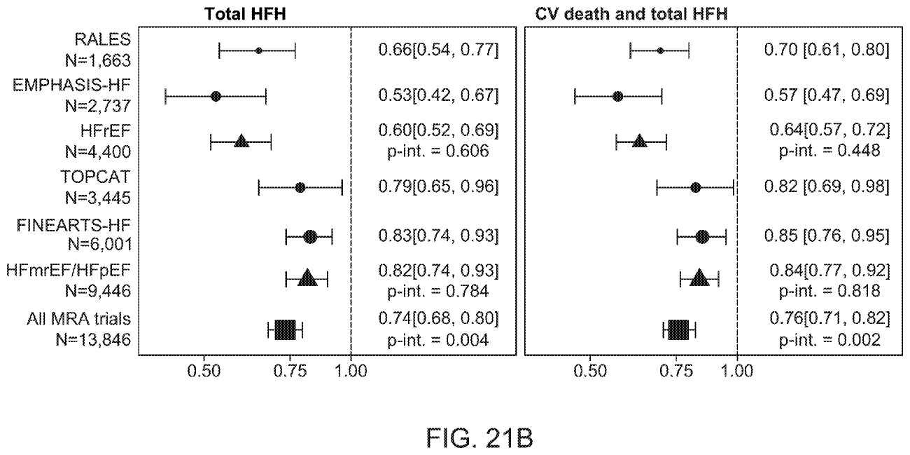

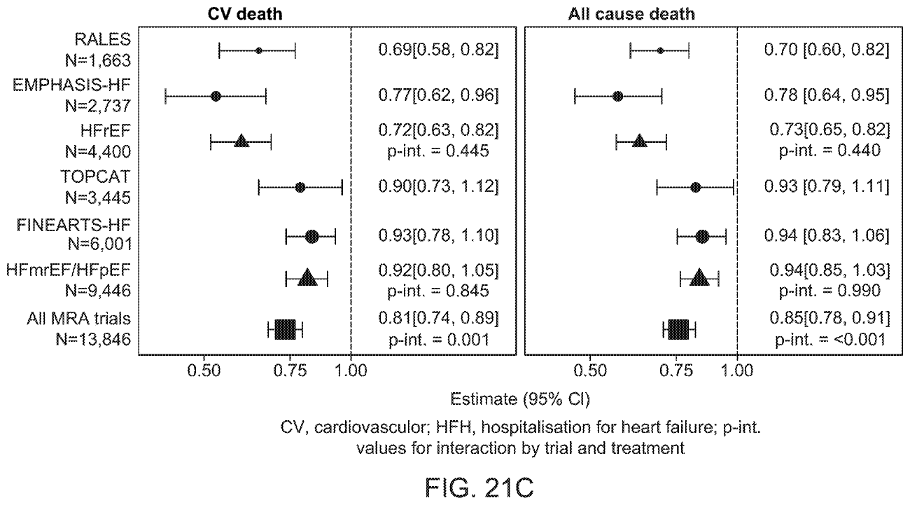

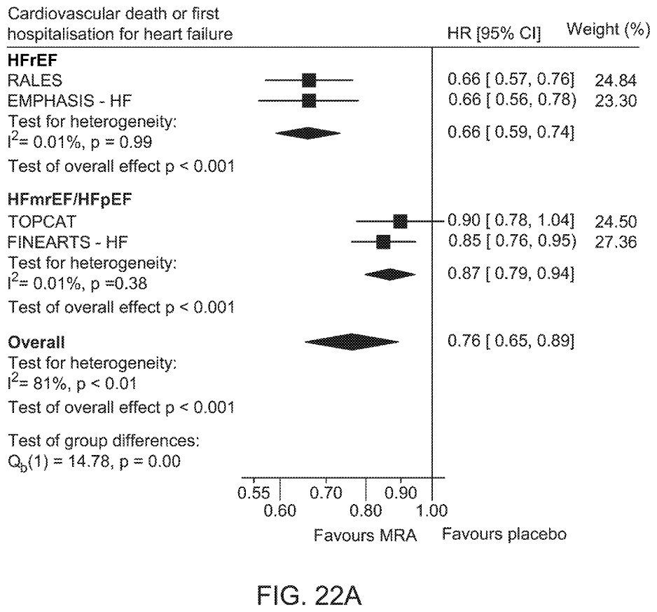

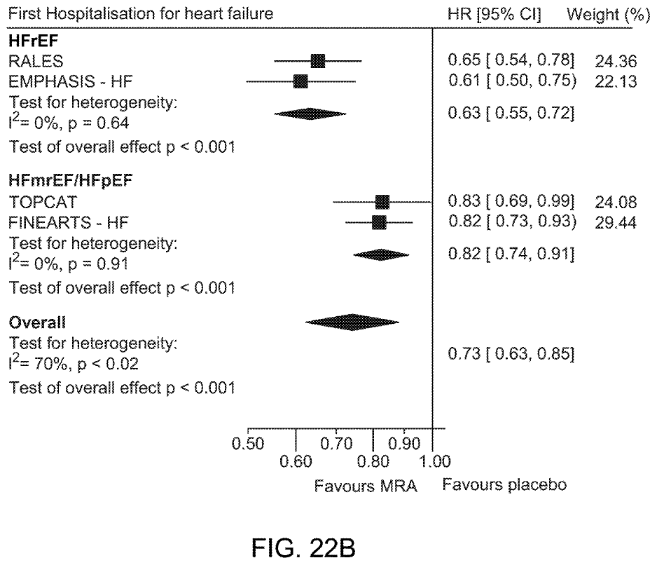

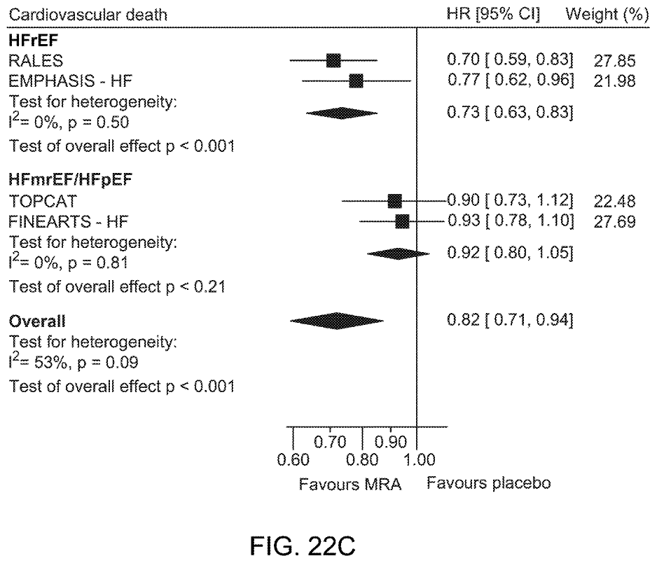

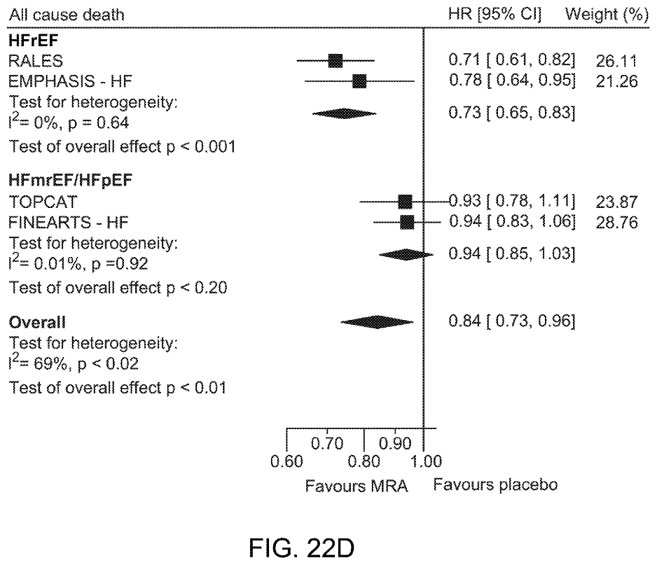

The rates of events in the placebo groups were higher in the trials including patients with HFrEF compared to those with HFmrEF/HFpEF (FIGS. 17A-17L, FIGS. 18A-18B, FIGS. 19A-19B). In the analysis of the time-to-first occurrence of cardiovascular death or HF hospitalization, including all four trials, the hazard ratio (HR) for an MRA compared to placebo was 0.77 (95% CI 0.72-0.83) (FIGS. 20A-20B, FIGS. 21A-21C). However, there was a statistically significant interaction between trials and the effect of treatment (p for interaction <0.001,FIGS. 20A-20B) which was also observed in the two-stage meta-analysis (Cochrane's Q p<0.001, I²=83%), FIGS. 221A-22D. Inspection of the treatment estimates showed that the source of the interaction was the greater treatment efficacy in the HFrEF trials (pooled HFrEF trials HR 0.66 [95% CI 0.59-0.73]) compared to the HFmrEF/HFpEF trials (pooled HFmrEF/HFpEF trials HR 0.87 [95% CI 0.79-0.95]) (FIGS. 20A-20B, FIGS. 21A-21C). Among the HFrEF trials and the HFmrEF/HFpEF trials, as separate groups, there was no further heterogeneity (p for interaction HFrEF trials=0.97; HFmrEF/HFpEF trials=0.49).

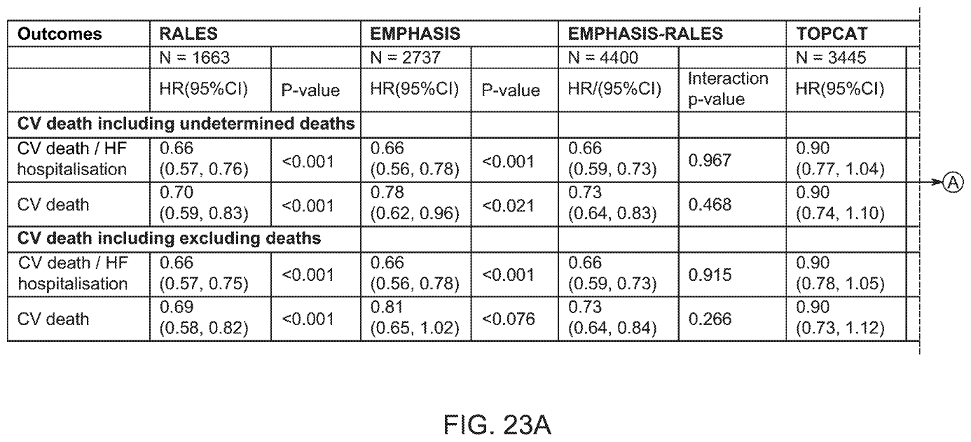

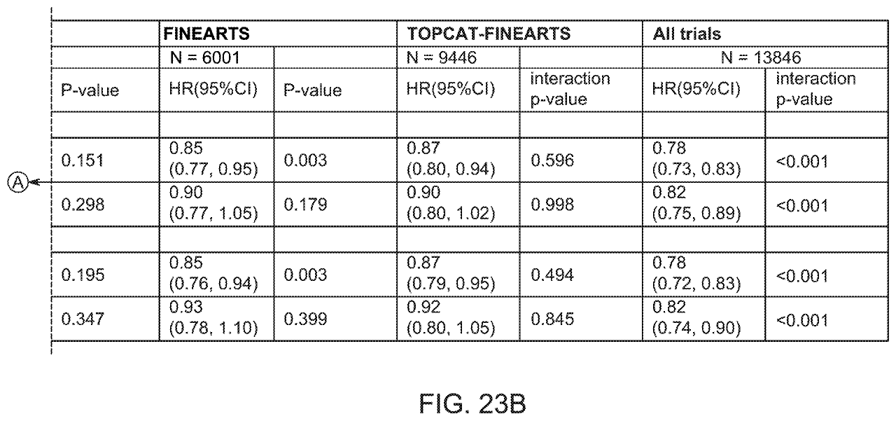

The same heterogeneity of treatment effect by trials was present for cardiovascular death, HF hospitalization, and all-cause death (FIGS. 20A-20B, FIGS. 21A-21C). The HR for the effect of an MRA on cardiovascular death in the two HFrEF trials was 0.72 (95% CI 0.63-0.82) and in the two HFmrEF/HFpEF trials was 0.92 (95% CI 0.80-1.05). The p for interaction over all trials was <0.001 and the p for interaction in the HFrEF trials was 0.45 and in the HFmrEF/HFpEF trials, it was 0.85 (FIGS. 20A-20B, FIGS. 21A-21C). For outcomes incorporating cardiovascular death, in sensitivity analyses including or excluding undetermined causes of death, the findings were similar (FIGS. 23A-23B). The HR for the effect of an MRA on a first HF hospitalization was 0.63 (95% CI 0.56-0.72) in the HFrEF trials and 0.82 (95% CI 0.74-0.91) in the HFmrEF/HFpEF trials (p for interaction<0.001) (FIGS. 20A-20B, FIGS. 21A-21C). There was no further heterogeneity in these two sets of trials (p for interaction HFrEF trials=0.66; HFmrEF/HFpEF trials=0.93).

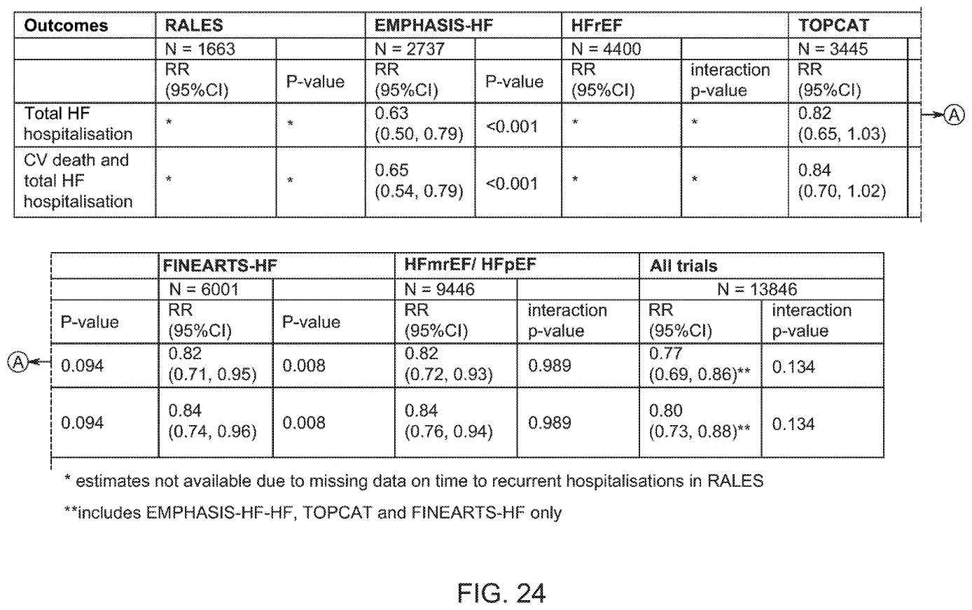

The rate ratio (RR) for the effect of an MRA on total HF hospitalizations was 0.60 (95% CI 0.52-0.69) in the HFrEF trials and 0.82 (95% CI 0.74-0.90) in the HFmrEF/HFpEF trials (p for interaction=0. 0.004) again with no interaction within the groups of trials (HFrEF trials=0.61; HFmrEF/HFpEF trials=0.78) (FIGS. 20A-20B). We observed similar results when total HF hospitalizations and cardiovascular deaths were examined: RR 0.64 95% CI 0.57-0.72 in the HFrEF trials and RR 0.84 95% CI 0.77-0.92 in the HFmrEF/HFpEF trials (p for interaction=0.001), with no further heterogeneity within the groups of trials (p for interaction HFrEF trials 0.45; HFmrEF/HFpEF trials=0.82) (FIGS. 20A-20B). Results were similar using a semi-parametric proportional rates model (FIG. 24).

The HR for the effect of an MRA on all-cause death in the HFrEF trials was 0.73 95% CI 0.65-0.82 and 0.94 95% CI 0.85-1.03 in the HFmrEF/HFpEF trials (p for interaction<0.001) with no further heterogeneity between the two groups of trials (p for interaction HFrEF trials=0.44; HFmrEF/HFpEF trials=0.99) (FIGS. 20A-20B, FIGS. 21A-21C).

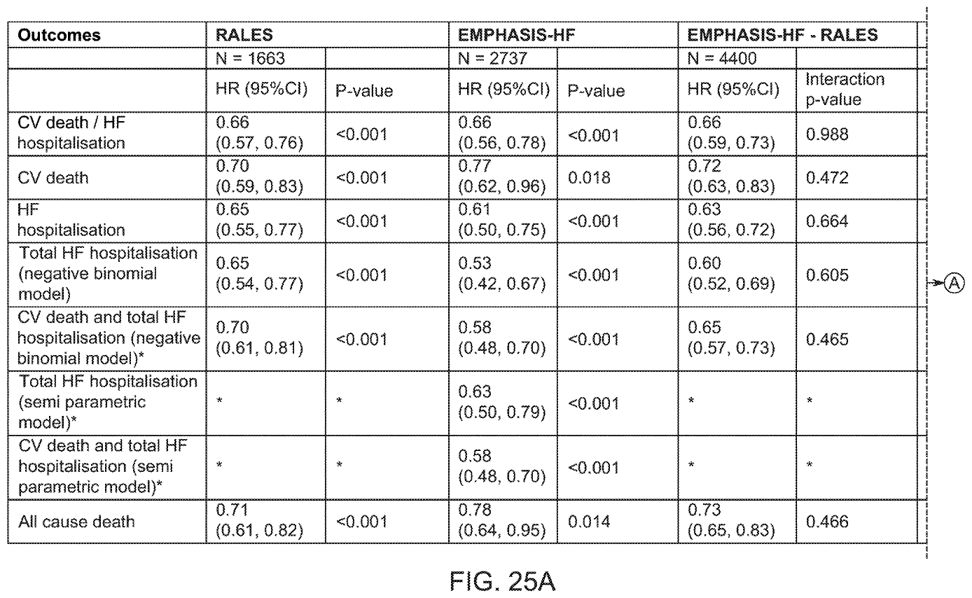

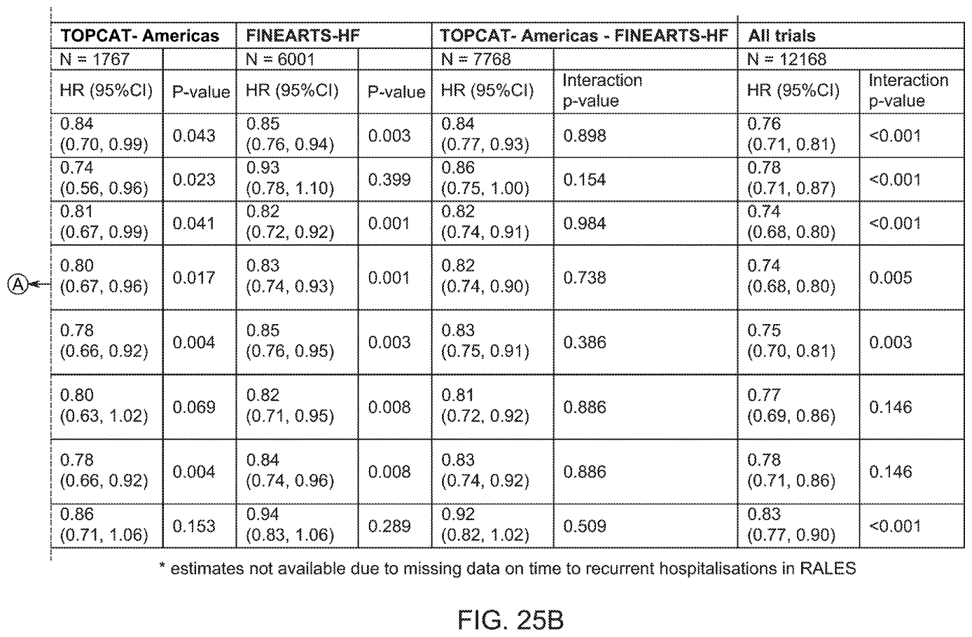

Given the interaction of the treatment effects by trial, the analyses of steroidal versus non-steroidal MRAs on outcomes were not conducted. A sensitivity analysis with a two-stage meta-analysis confirmed the results from the one-stage individual patient-level analysis (FIGS. 21A-21C). The results of the individual patient level meta-analysis were unchanged in a sensitivity analysis using only the patients randomized in the Americas in TOPCAT (FIGS. 25A-25B) with a hazard ratio estimate for cardiovascular death or HF hospitalization of 0.84 (95% CI 0.77-0.93), heart failure hospitalization of 0.82 (95% CI 0.74-0.91), and cardiovascular death of 0.86 (95% CI 0.75-1.00) in patients with HFmrEF/HFpEF.

### Subgroups

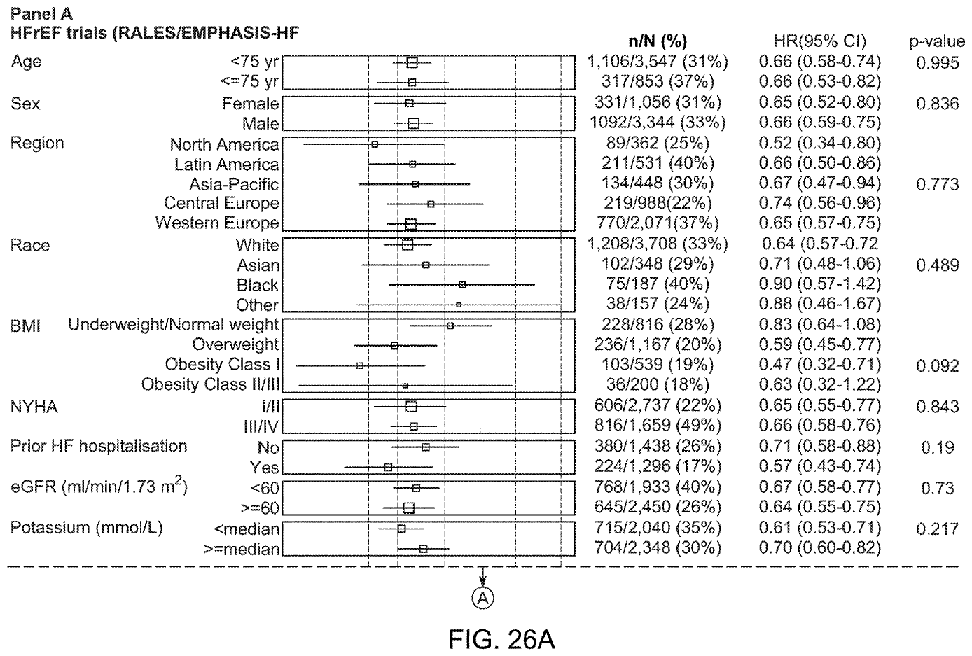

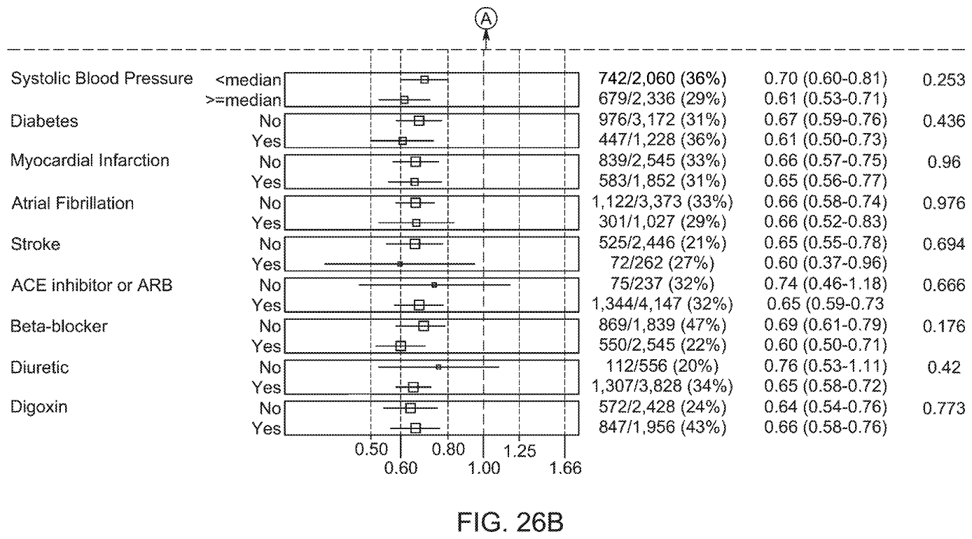

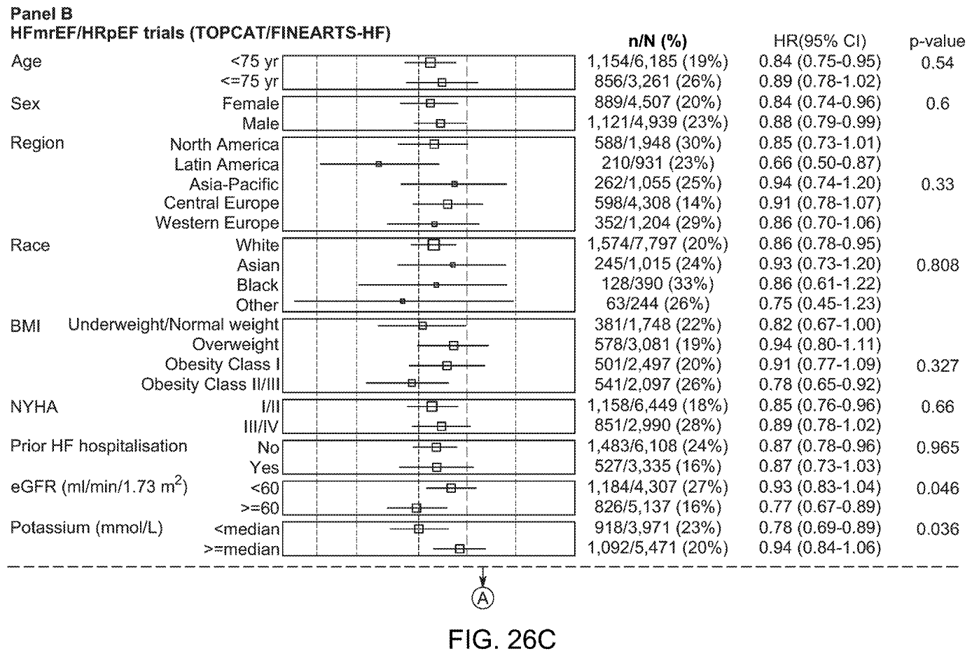

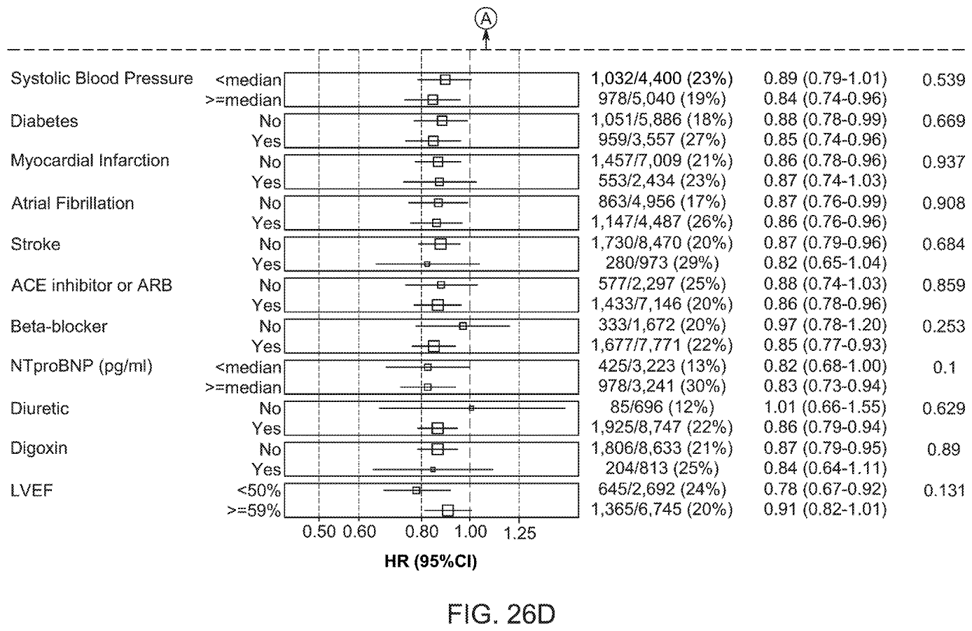

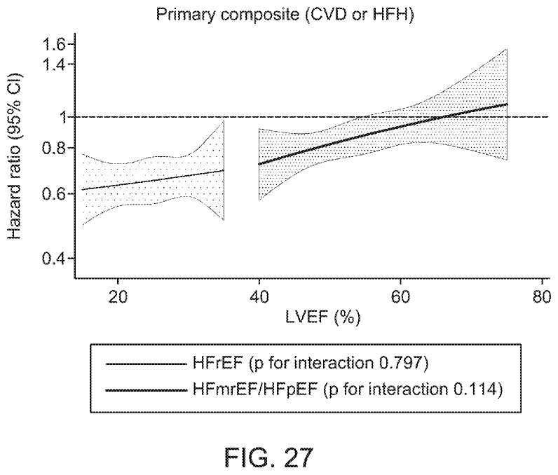

The effect of therapy was consistent across all subgroups in the HFrEF and HFmrEF/HFpEF trials (FIGS. 26A-26D). In a subgroup analysis of all four trials, there was an interaction between baseline potassium and treatment efficacy with greater efficacy in those with a lower potassium (FIG. 24). The interaction with ejection fraction as a categorical variable was similar to the interaction between the trials. When the interaction between therapy and ejection fraction was modelled as a continuous variable within the HFrEF and HFmrEF/HFpEF trials separately, there was no treatment heterogeneity confirming that the majority of the heterogeneity was between HFrEF and HFmrEF/HFpEF (FIG. 27).

### Safety

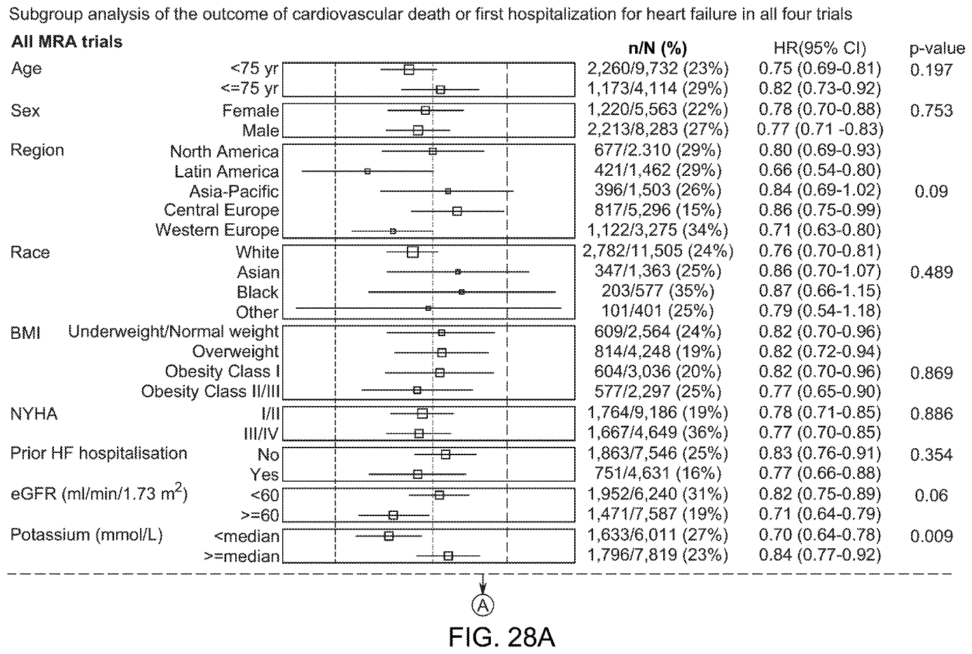

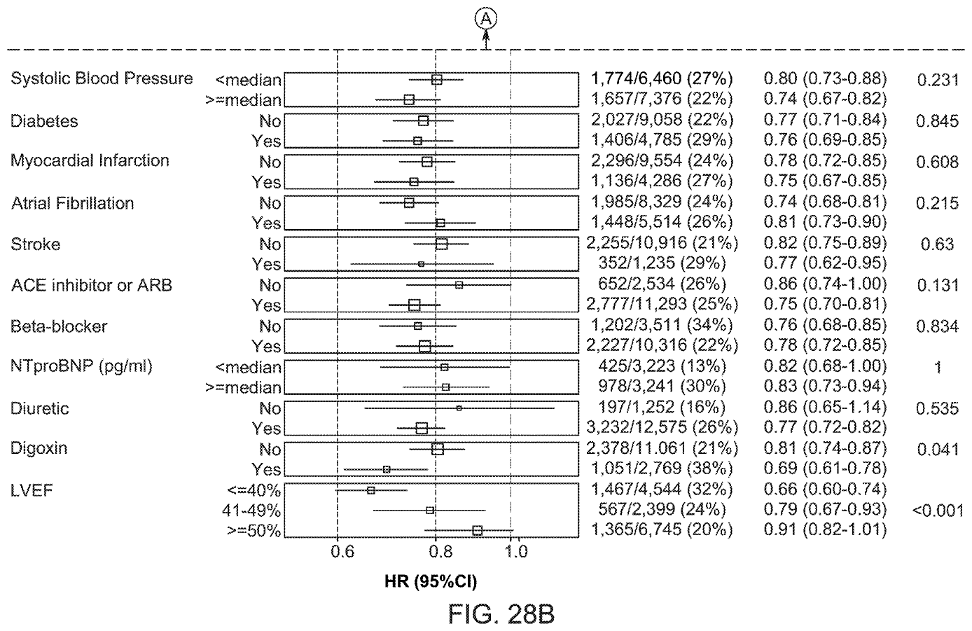

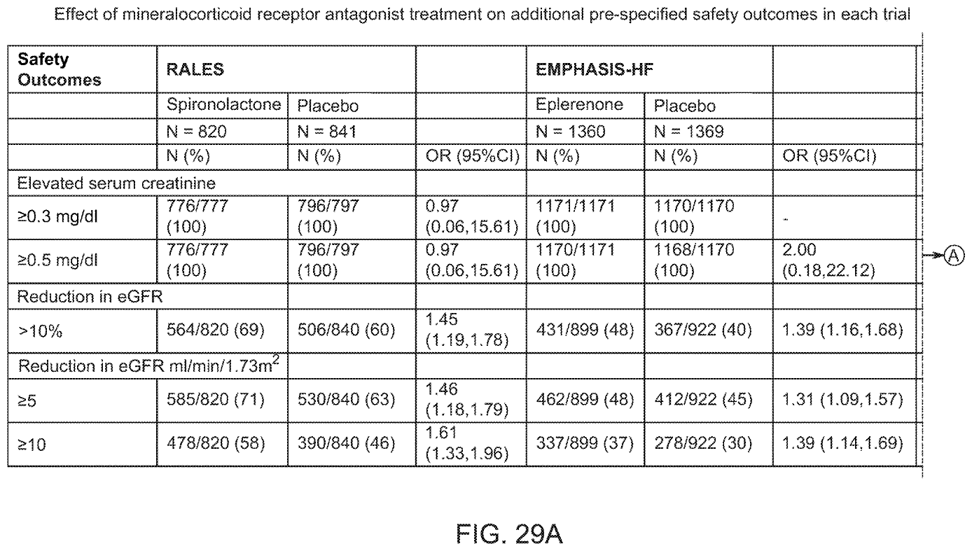

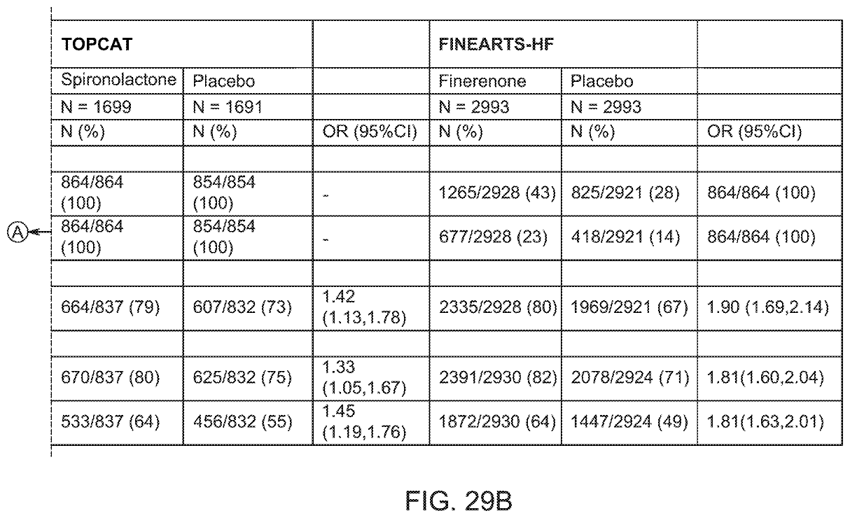

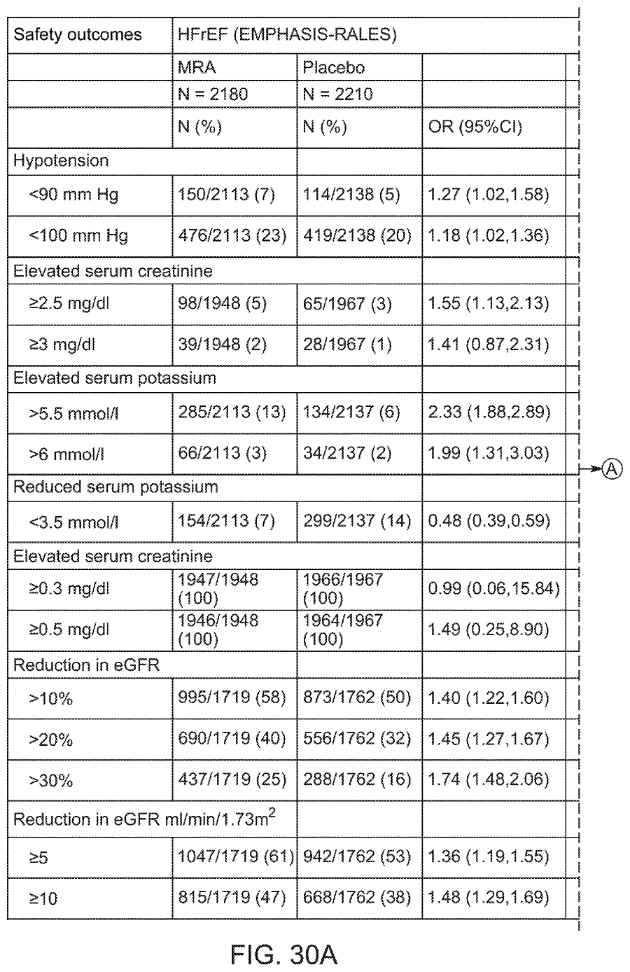

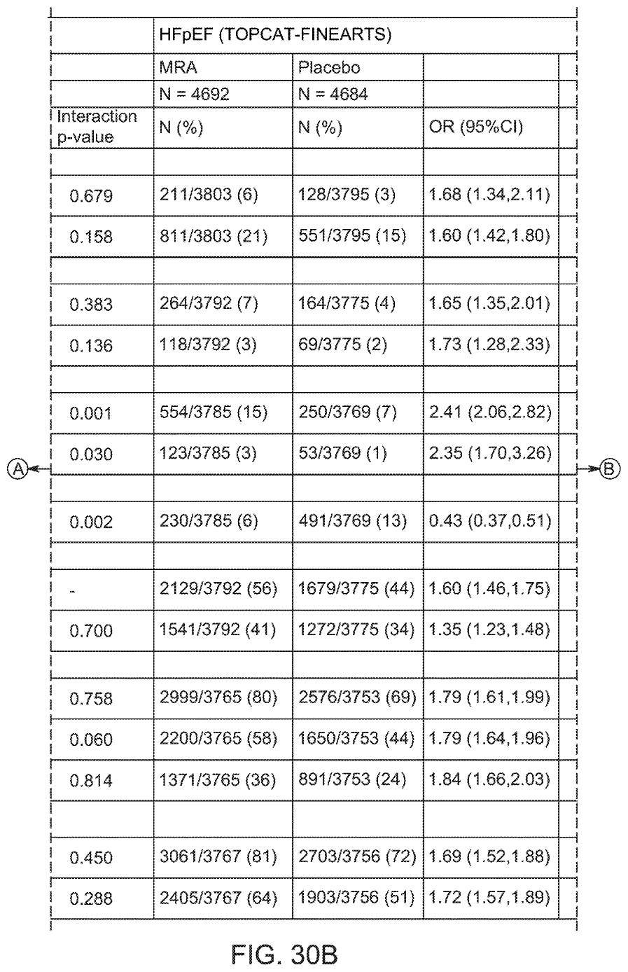

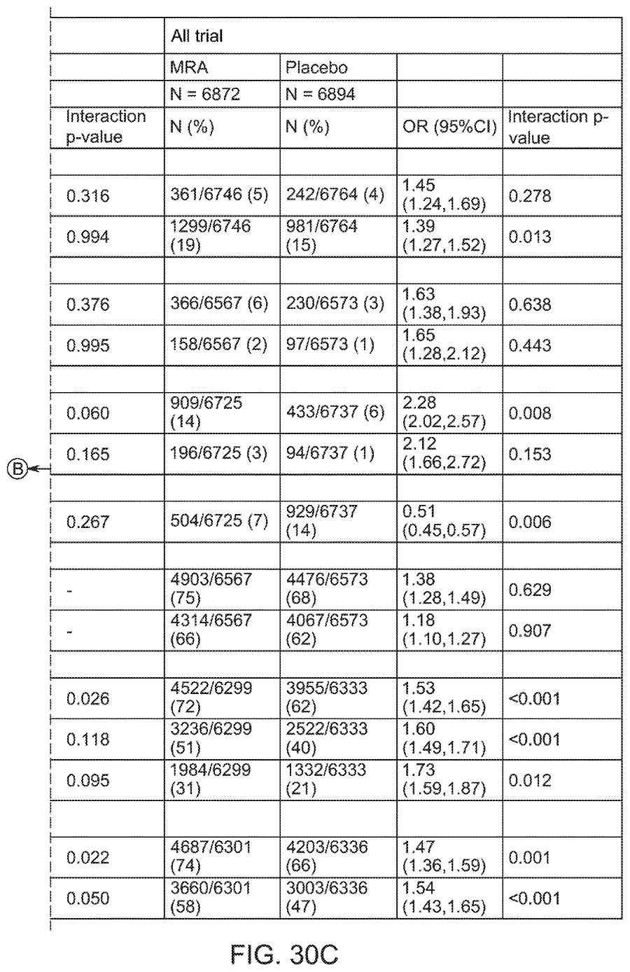

Safety outcomes in each trial and treatment arm in patients receiving trial treatment are shown in. The risk of hyperkalemia defined as serum potassium >5.5 (moderate) or >6 mmol/L (severe) was twice that of the placebo group. However, the absolute risk of severe hyperkalemia was low at approximately 2.9% in the MRA group and 1.4% in the placebo group. There was little heterogeneity in the risk of any hyperkalemia when rates were examined by trials defined by ejection fraction or by type of MRA (FIGS. 25A-25B, FIGS. 28A-28B, FIGS. 29A-29B, FIGS. 30A-30C), however, this analysis did not differentiate between on-and off-treatment hyperkalemia. Conversely, the risk of hypokalaemia (potassium <3.5 mmol/L) was halved in MRA-treated patients compared to placebo-treated patients (7% versus 14%, respectively) across all trials (FIGS. 30A-30C).

Although hypotension was more common in the low ejection fraction trials we found no statistical heterogeneity for the effect of treatment on a systolic blood pressure <100 mmHg by trial group (7% versus 5% in the HFrEF trials and 5% versus 3% in the HFmrEF/HFpEF trials).

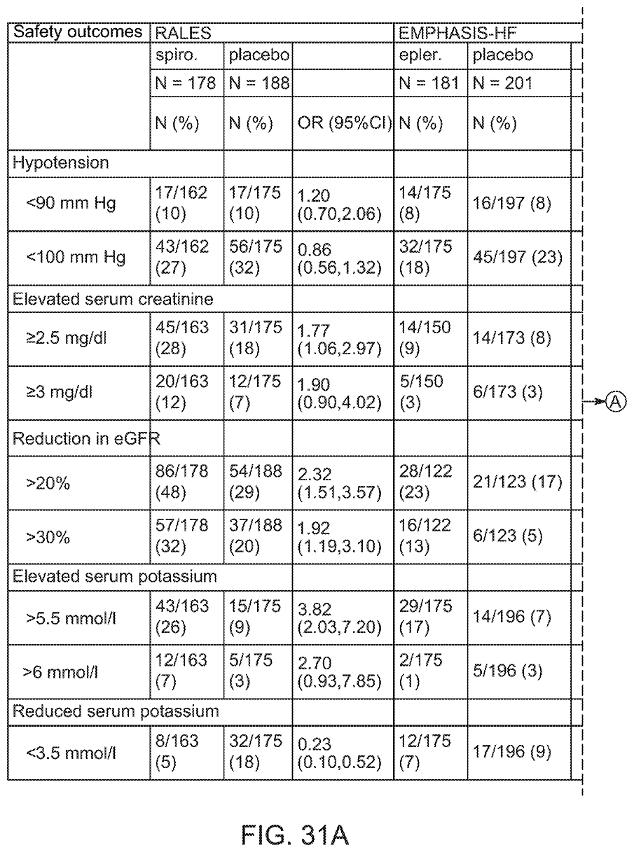

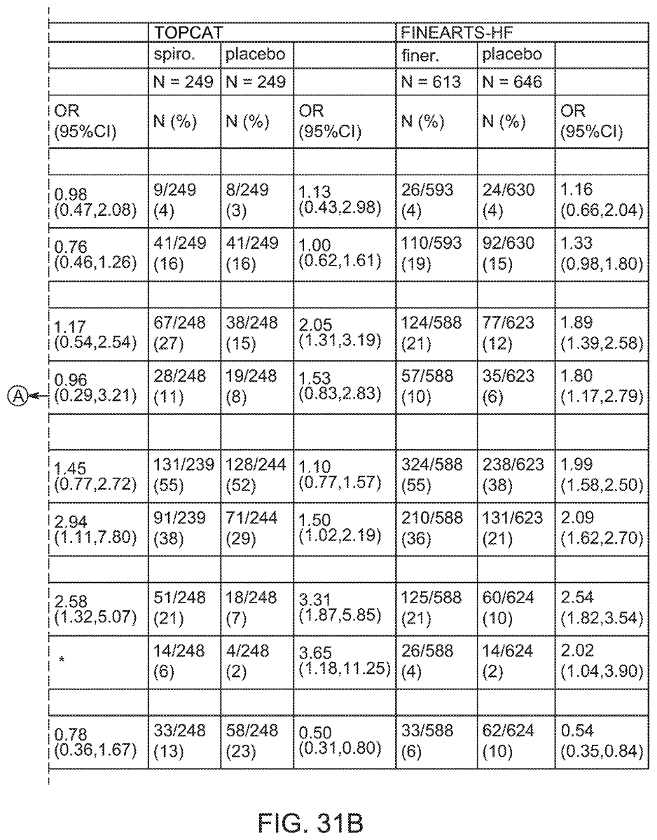

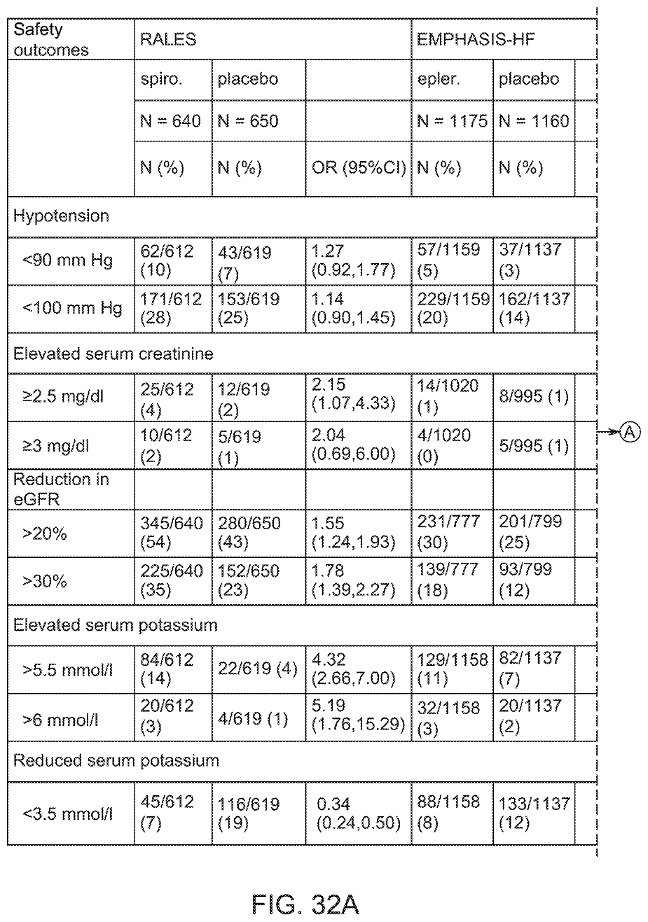

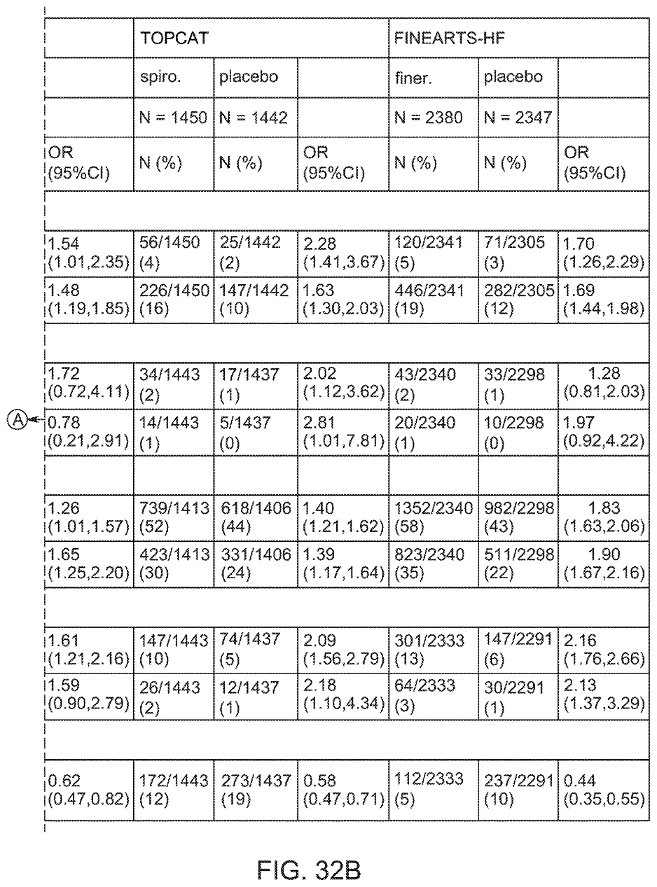

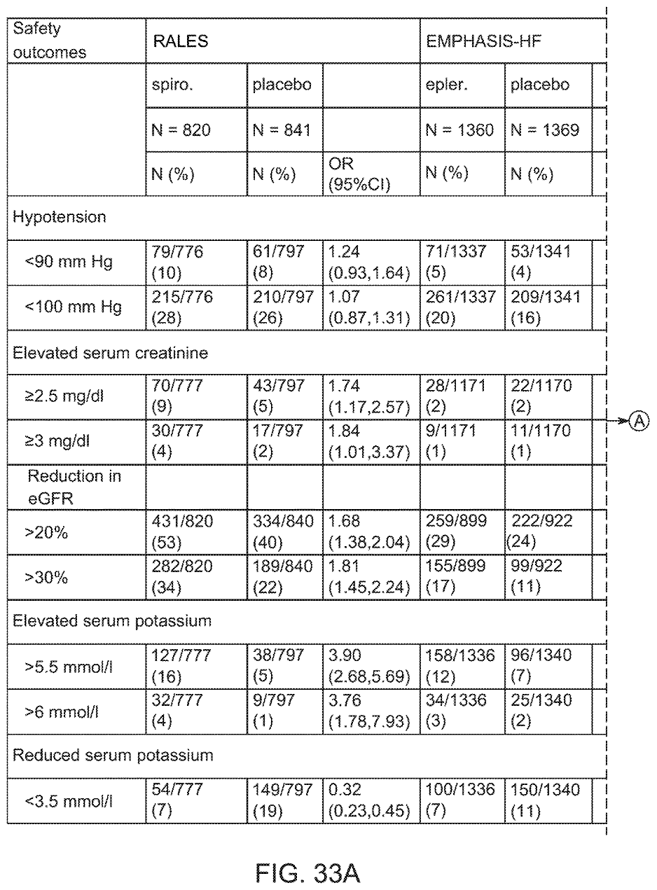

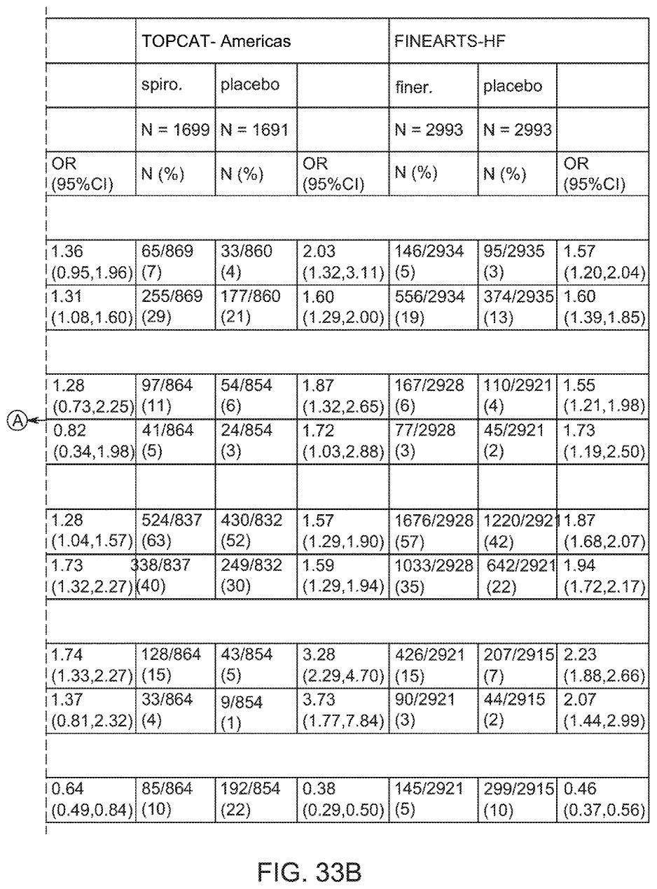

Additional safety outcomes are given by baseline eGFR and using TOPCAT America (FIGS. 31A-31B, FIGS. 32A-32B, FIGS. 33A-33B).

### Discussion

Before this meta-analysis, there was strong evidence of the benefits of the steroidal MRAs spironolactone and eplerenone in patients with heart failure and a substantially reduced left ventricular ejection fraction but uncertainty about the efficacy of MRAs in patients with mildly reduced or preserved ejection fraction, especially as spironolactone failed to show significant benefit the TOPCAT trial. With completion of the FINEARTS-HF trial using finerenone, there is now evidence that blocking the mineralocorticoid receptor with a non-steroidal MRA is beneficial in people with an ejection fraction of 40% or above. In this meta-analysis, collectively, all four major trials demonstrate the value of steroidal and non-steroidal MRAs in heart failure irrespective of ejection fraction phenotype.

However, this meta-analysis also shows that there was significant heterogeneity between the treatment effect on efficacy outcomes in the trials that included patients with reduced left ventricular ejection fraction compared to the trials in people with mildly reduced or preserved ejection fraction, with smaller relative risk reductions in the latter participants. This variation in efficacy according to ejection fraction phenotype was evident for both heart failure hospitalization and mortality, although there was a significant reduction in heart failure hospitalization with MRAs across all ejection fraction phenotypes. By contrast, cardiovascular death was not reduced significantly in patients with mildly reduced or preserved ejection fraction and this conclusion was not altered by using different definitions of cardiovascular death (i.e., whether deaths of unknown cause were included or excluded) or including only TOPCAT patients enrolled in the Americas, although in the latter sensitivity analysis, the HR was 0.86 with 95% CI 0.75-1.00. This differential effect on fatal and non-fatal outcomes was also observed in the sodium-glucose co-transporter 2 trials and with sacubitril/valsartan. The lack of effect of all of these treatments on all-cause death in patients with a higher ejection fraction may be due to the much larger proportion of non-cardiovascular deaths in this group compared to people with lower ejection fraction, with non-cardiovascular deaths unlikely to be reduced by treatments such as an MRA. Among patients with mildly reduced or preserved ejection fraction, other treatments acting through neurohumoral mechanisms show attenuated efficacy in patients with a completely normal ejection fraction. While our analyses by ejection fraction category or using ejection fraction as a continuous variable might suggest this visually, formal interaction testing did not show that ejection fraction modified the effects of MRAs in patients with mildly reduced or preserved ejection fraction.

Examination of other subgroups including by age, comorbidity, and laboratory and other physiological variables showed mainly consistent results for the effect of an MRA on the primary outcome in both the reduced ejection fraction trials and, separately, in the mildly reduced or preserved ejection fraction trials (and, therefore, across all four trials). Importantly, the meta-analysis included a substantial number of patients with significantly impaired kidney function and the benefit of MRA treatment was consistent in these patients as well as those with better baseline kidney function.

Hyperkalemia is a safety concern with MRAs, and the risk of both modest and severe hyperkalemia was approximately doubled with MRA treatment in the reduced ejection fraction trials and this risk was elevated to a similar extent in the mildly reduced/preserved ejection fraction trials. However, the absolute risk of serious hyperkalemia (potassium >6 mmol/L) was low (around 3 percent in the MRA treatment group compared with 1 to 1.5 percent in the placebo group). We did not find heterogeneity between trials or between MRAs for hyperkalemia. Hypokalemia is a more common problem than hyperkalemia in heart failure because of the use of diuretics and it is at least as important a safety concern. The overall risk of hypokalaemia (potassium <3.5 mmol/L) was halved by MRA treatment (7% in the MRA group versus 14% in the placebo group).

Initiation of an MRA may also lead to a decline in estimated glomerular filtration rate although decreases in glomerular filtration rate are extremely common in patients with heart failure as evidenced by the rate of these changes in the placebo group in all four trials. The risk of a 20 or 30 percent decrease in glomerular filtration rate was between one and a half and two times as common with mineralocorticoid receptor antagonist treatment compared to placebo.

Despite possible concerns about hypotension, a systolic blood pressure below 90 mmHg was uncommon overall and the difference in episodes of low systolic blood pressure between MRA treatment and placebo was small.

The results of these analyses should be interpreted with some limitations in mind. Certain variables were not available in individual trials e.g., body mass index in RALES. In RALES the time to hospitalization was only available for the first event and therefore a negative binomial model had to be used to analyse repeat events. The FINEARTS-HF trial included episodes of worsening heart failure requiring urgent treatment as an equivalent to hospitalization. These were not collected in the older trials but have been validated endpoints in recent trials because thresholds for hospital admission have changed (although they constituted a minority of events). Our analysis was restricted to randomized controlled trials with more than 1,000 participants and the exclusion of smaller trials might affect the generalisability of this meta-analysis, as the included trials had relatively homogenous exclusion criteria. While we tried to control for the difference in baseline risk by stratifying by trial in our models the level of heterogeneity was high as demonstrated both in the individual patient-level analysis and our two-stage meta-analysis. However, this meant that we were not able to perform a direct comparison between steroidal MRAs of spironolactone and eplerenone with the non-steroidal MRA finerenone. The meta-analysis includes trials conducted over several decades with changes in background care in that period. Sodium-glucose co-transporter 2 inhibitors are now considered a foundational treatment for heart failure but were not developed or indicated at the time of the older trials and we cannot comment on the concurrent use of an MRA and these drugs that were introduced as a treatment for heart failure during the conduct of the FINEARTS-HF trial.

In conclusion, this systematic review and meta-analysis of nearly 14,000 patients across four large clinical trials provide comprehensive evidence that steroidal and non-steroidal MRAs improve cardiovascular outcomes in patients with heart failure, regardless of ejection fraction phenotype. These benefits were consistent across patient subgroups. Treatment with an MRA should be considered in all patients with heart failure without a contraindication to this treatment.

### Example 16—Effect of Finerenone in Patients With HFmrEF/HFpEF According to Body Mass Index: A Prespecified Analysis of the FINEARTS-HF Trial

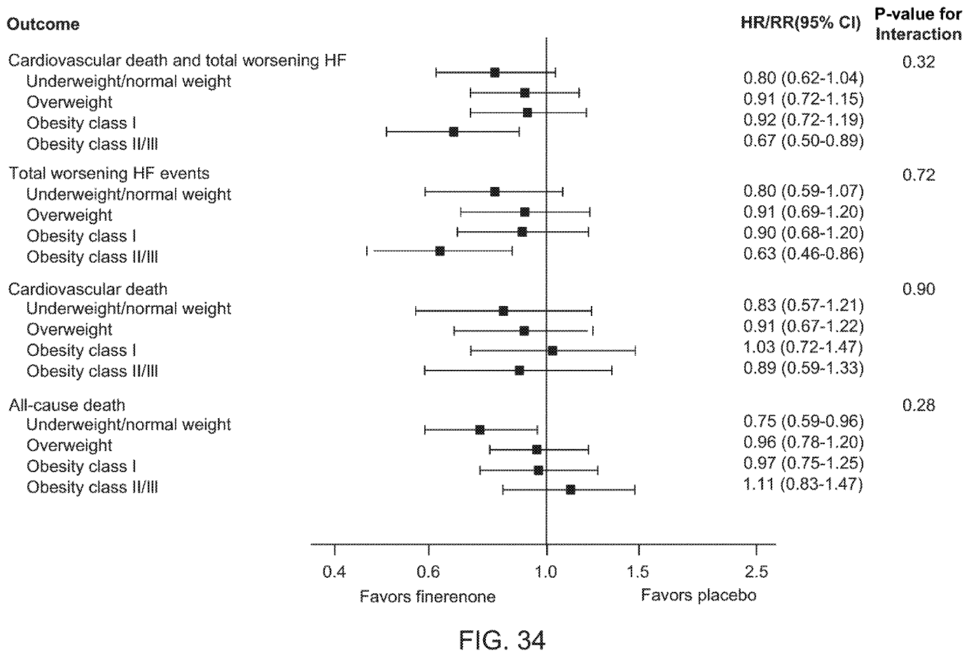

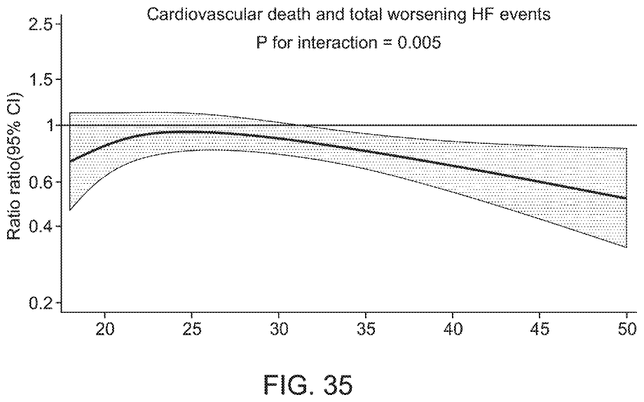

Introduction: Obesity is associated with excessive adipocyte-derived aldosterone secretion, independent of the classical renin-angiotensin-aldosterone cascade, and MRAs may be more effective in obese patients with HF. We examined the effects of the non-steroidal MRA, finerenone, compared to placebo, according to BMI in FINEARTS-HF. See FIG. 34 and FIG. 35.

Methods: A total of 6,001 patients with HF with NYHA functional class II-IV, LVEF >40%, evidence of structural heart disease, and elevated NT-proBNP levels were randomized to finerenone or placebo. BMI (kg/m²) was examined using standard categories, i.e., underweight/normal weight (<24.9; n=1,306); overweight (25.0-29.9; n=1,990); obesity class I (30.0-34.9; n=1,546); obesity class II (35.0-39.9; n=751); obesity class III (>40; n=395). The primary outcome was CV death and total worsening HF events.

Results: Data on BMI were available in 5,988 patients (median 29.2 [IQR 25.5-33.6]). Compared to patients who were underweight/normal weight, those with a higher BMI were younger and more likely to be women, and they had higher systolic blood pressure and LVEF. Patients with higher BMI had lower NT-proBNP levels and worse NYHA functional class and KCCQ scores, and they were less likely to have a prior HF hospitalization but more likely to have diabetes. There was no difference in the use of SGLT2i across BMI categories, but patients with higher BMI were more likely to be treated with a loop diuretic and less often with ARNI. Compared to patients who were underweight/normal weight, those who were obese had a higher risk of the primary outcome (underweight/normal weight, reference; overweight, unadjusted RR 0.96 [95% CI, 0.81-1.15]; obesity class I, 1.04 [0.86-1.26]; obesity class II-III, 1.26 [1.03-1.54]). The effect of finerenone on the primary outcome did not vary by baseline BMI (underweight/normal weight, RR 0.80 [95% CI, 0.62-1.04]; overweight, 0.91 [0.72-1.15]; obesity class I, 0.92 [0.72-1.19]; obesity class II-III, 0.67 [0.50-0.89]; Pinteraction=0.32). However, when BMI was examined as a continuous variable, the beneficial effect of finerenone appeared to be greater in those with higher BMI (Pinteraction=0.005). A similar pattern was observed for total worsening HF events. Consistent effects were observed for CV and all-cause death, and improvement in the KCCQ-TSS.

Conclusions: In FINEARTS-HF, the beneficial effects of finerenone on clinical events and symptoms were consistent, irrespective of BMI at baseline, possibly with a greater effect on worsening HF events with higher BMI.

### Example 17—Efficacy and Safety of Finerenone in Patients with Heart Failure and Mildly Reduced or Preserved Ejection Fraction: a Prespecified Sex-Specific Analysis of the FINEARTS-HF Trial

Sex-related differences are recognized in the clinical presentation, symptoms and quality of life, prognosis, and treatment response in heart failure (HF). Therefore, it is important to evaluate the efficacy and safety of new treatments for HF in both women and men.

FINEARTS-HF was a randomized, double-blind, multicenter, event-driven, trial in patients with left ventricular ejection fraction ≥40%, investigating the efficacy and safety of the non-steroidal mineralocorticoid receptor antagonist finerenone, compared to placebo, in patients with HF and mildly reduced or preserved ejection fraction (HFmrEF/HFpEF). The primary outcome was the composite of cardiovascular death and total (first and recurrent) HF events (either an unplanned HF hospitalization or an urgent HF visit). In a prespecified subgroup analysis, we evaluated the efficacy and safety of finerenone compared to placebo in both women and men.

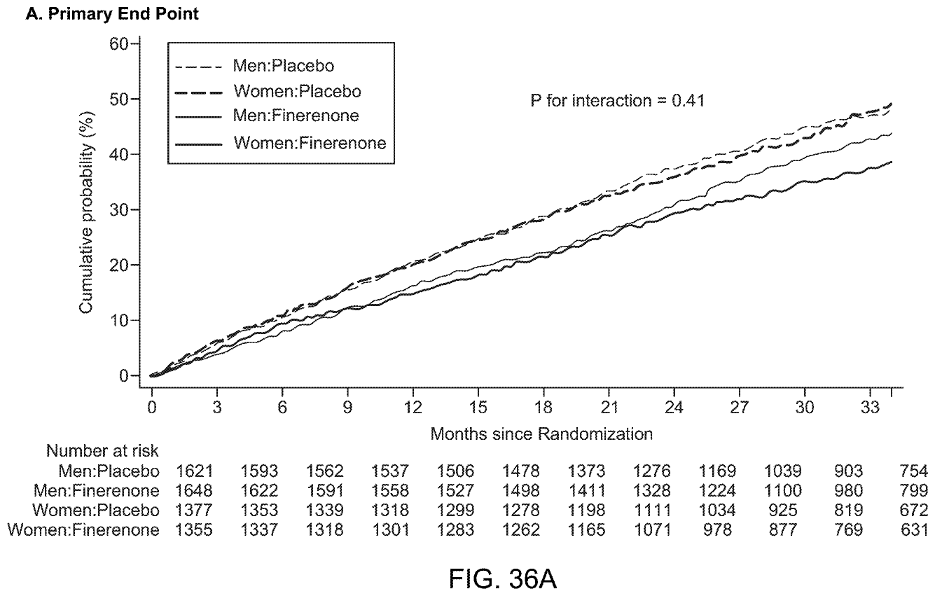

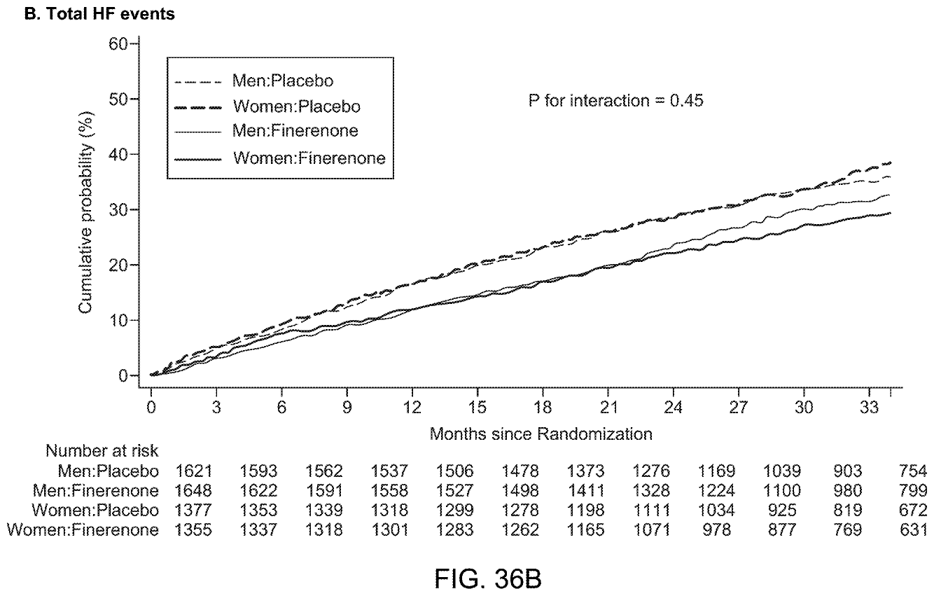

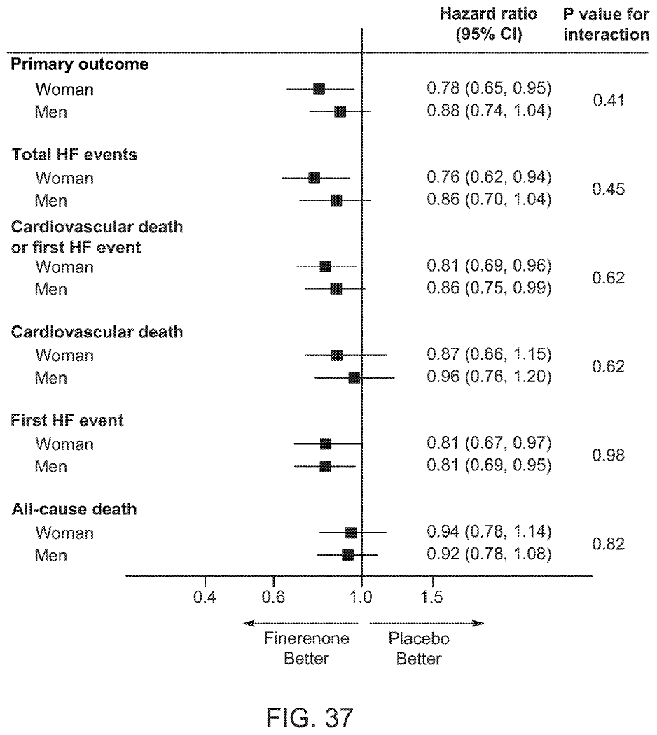

Overall, 2,732 women (45.5%) and 3,269 men (54.5%) were randomized. Women were older than men and had higher rates of obesity, lower estimated glomerular filtration rate, worse NYHA functional class, and lower Kansas City Cardiomyopathy Questionnaire-total symptom scores (KCCQ-TSS). The rate of the primary outcome in the placebo group was similar between women and men. Compared to placebo, finerenone reduced the risk of the primary outcome to a similar extent in both women and men, with rate ratios (RRs) of 0.78 (95% CI 0.65-0.95) in women versus 0.88 (0.74-1.04) in men, respectively (P for interaction=0.41). Consistent benefits were observed for the components of the primary outcome and all-cause death (FIGS. 36A-36B, FIG. 37). The mean increase (improvement) in KCCQ-TSS from baseline to 12 months was greater with finerenone, irrespective of sex (P for interaction=0.72). Adverse events of interest were similar in both women and men.

In FINEARTS-HF, finerenone was efficacious and safe, irrespective of sex, in patients with HFmrEF/HFpEF.

### Example 18—Initial Decline (Dip) in Estimated Glomerular Filtration Rate with Initiation of Finerenone in Patients with Mildly Reduced or Preserved Ejection Fraction: a Prespecified Analysis of FINEARTS-HF

An initial decline in estimated glomerular filtration rate (eGFR) often leads to reluctance to continue the life-saving therapy in patients with heart failure (HF). However, this early dip in eGFR has been shown to predict a favorable response to several HF therapies. We have analyzed the early change in eGFR and outcomes in patients treated with the non-steroidal mineralocorticoid receptor antagonist (MRA) finerenone.

FINEARTS-HF was a randomized, double-blind, multicenter, event-driven, trial in patients with left ventricular ejection fraction ≥40%, investigating the efficacy and safety of finerenone compared to placebo. We examined the association between initial decline (“dip”) in eGFR (≥15% as in prior studies) from randomization to 1 month and subsequent outcomes, in patients randomly assigned to finerenone or placebo. The primary outcome was the composite of total (first and repeat) HF events and cardiovascular death.

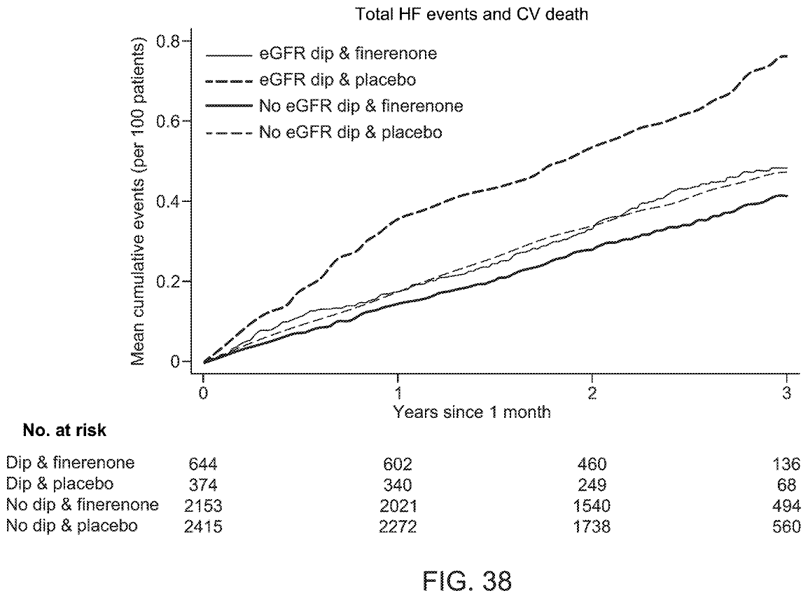

Among 5,587 patients with an eGFR measurement at baseline and 1 month, 1,018 (18.2%) experienced a ≥15% dip in eGFR (despite having a similar baseline eGFR to those who did not dip). The mean change in eGFR between randomisation and 1 month was −0.31 (95% CI, −0.68 to 0.06) ml/min/1.73 m² with placebo and −2.75 (95% CI, −3.16 to −2.34) ml/min/1.73 m² with finerenone, giving a between-treatment difference of 2.49 (95% CI, 3.03 to 1.96) ml/min/1.73 m². The proportion of patients experiencing ≥15% decline in eGFR was 12.2% with placebo and 20.6% with finerenone (odds ratio 1.87 [95% CI, 1.62-2.15], P<0.001). An initial decline in eGFR (>15%) was associated with a higher risk of the primary outcome in patients assigned to placebo (rate ratio 1.59 [95% CI: 1.24-2.04]) but not in those assigned to finerenone (rate ratio 1.09 [0.87-1.38], Pinteraction<0.001) (FIG. 38). Furthermore, the efficacy of finerenone was maintained in patients experiencing an early decline in eGFR, as was safety, including the incidence of hyperkalemia (potassium >5.5 and >6.0 mmol/L) (Pinteraction=0.20 and 0.51, respectively). A similar pattern was observed when larger (20%) and smaller (10%) dips in eGFR were analyzed.

Although an initial dip in eGFR was associated with worse subsequent outcomes in patients assigned to placebo, this relationship was not seen in those treated with finerenone. As with other HF treatments, an early decline in eGFR should not automatically lead to the discontinuation of finerenone in patients with HF.

### Example 19—Effect of Finerenone on a Hierarchical Composite Endpoint Analyzed Using Win Statistics in Patients with Heart Failure and Mildly Reduced or Preserved Ejection Fraction: a Prespecified Analysis of FINEARTS-HF

FINEARTS-HF showed the efficacy of finerenone in reducing cardiovascular death and total (first and repeat) heart failure (HF) events (either an unplanned hospitalization for HF or an urgent HF visit), compared to placebo, in patients with HFmrEF/HFpEF. Win statistics allow analysis of these events according to their clinical importance.

FINEARTS-HF enrolled patients with New York Heart Association functional class II-IV symptoms, a left ventricular ejection fraction ≥40%, and evidence of structural heart disease. We developed a prespecified hierarchical composite endpoint including the components of the original primary outcome, analyzed in the following order: cardiovascular death (Tier 1); total HF hospitalizations (taking account of both the number of events and time to event) (Tier 2); and total urgent HF visits (Tier 3). Three win statistics were calculated: win ratio (ratio of wins to losses), win odds (modification of win ratio that accounts for ties and corresponds to the odds that the active treatment group has a better outcome than the control group), and net benefit (corresponds to the absolute risk difference). Because win statistics are affected by the duration of follow-up and the censoring distribution, we assessed the hierarchical composite outcome over a fixed period of 24 months.

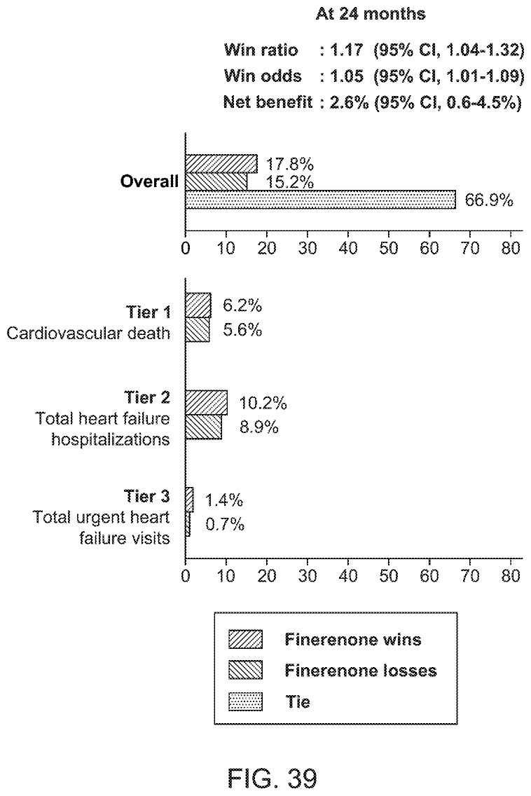

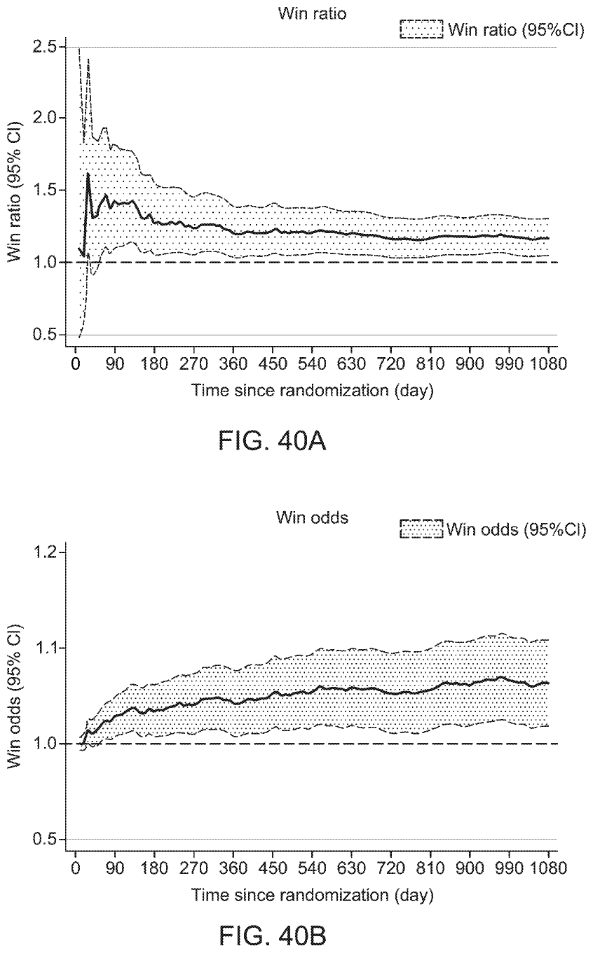

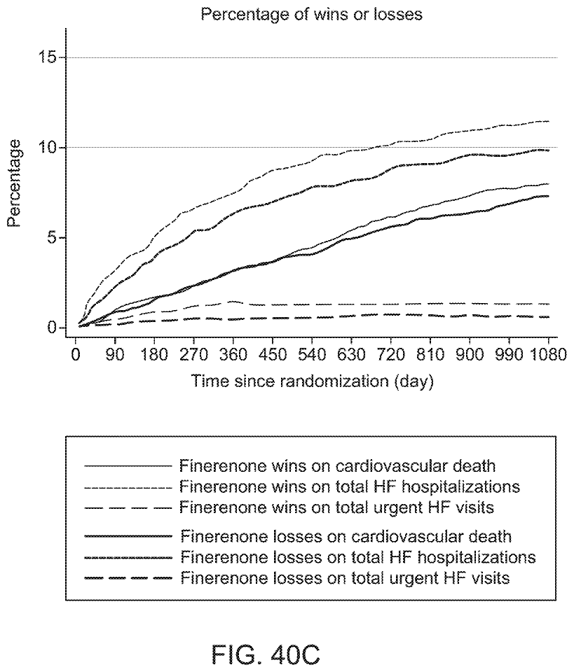

The 6,001 participants were randomized equally to finerenone (n=3,003) or placebo (n=2,998). At 24 months, a total of 825 cardiovascular deaths and worsening HF events were observed in the finerenone group, compared with 1,012 events in the placebo group. The win ratio was 1.17 (95% CI 1.04-1.32) (p=0.01), demonstrating more wins than losses in the finerenone group (FIG. 39). The win odds were 1.05 (95% CI 1.01-1.09), and the net benefit was 2.6% (95% CI 0.6-4.5%). The win ratio remained above 1.0 from 60 days after randomization and reached a plateau after approximately 12 months (FIGS. 40A-40C). The difference between wins and losses for total HF hospitalizations contributed more to the overall difference between wins and losses than cardiovascular death.

Finerenone treatment led to a significant improvement in a composite hierarchical outcome that incorporated cardiovascular death, total HF hospitalizations, and total urgent HF visits, with early onset of benefit.

### Example 20—Finerenone and Risk of Hyperkalemia in Patients with Heart failure with Mildly Reduced or Preserved Ejection Fraction

Finerenone, a non-steroidal mineralocorticoid receptor antagonist (MRA), reduced the risk of cardiovascular death and total worsening heart failure (HF) events in patients with HF with mildly reduced or preserved ejection fraction in FINEARTS-HF. We investigated the frequency and predictors of hyperkalemia, and examined the treatment effect of finerenone, relative to placebo, on clinical outcomes based on post-randomization potassium levels.

FINEARTS-HF was a multicenter, randomized, double-blind trial comparing finerenone (titrated to 20 or 40 mg, depending on baseline kidney function) versus placebo in patients with HF with LVEF ≥40%, structural heart disease, and elevated natriuretic peptide levels. Per protocol, investigators down-titrated or temporarily interrupted study medication if potassium levels were ≥5.5 mmol/L. The primary endpoint was the composite of total worsening HF events and cardiovascular death. Landmark analyses were conducted to examine risks of the primary endpoint subsequent to 1-month measurements of potassium levels separately in each treatment arm.

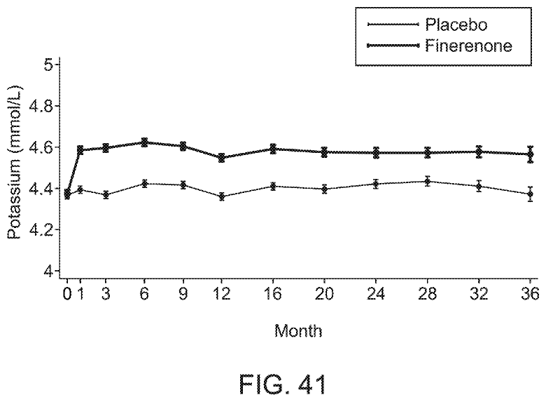

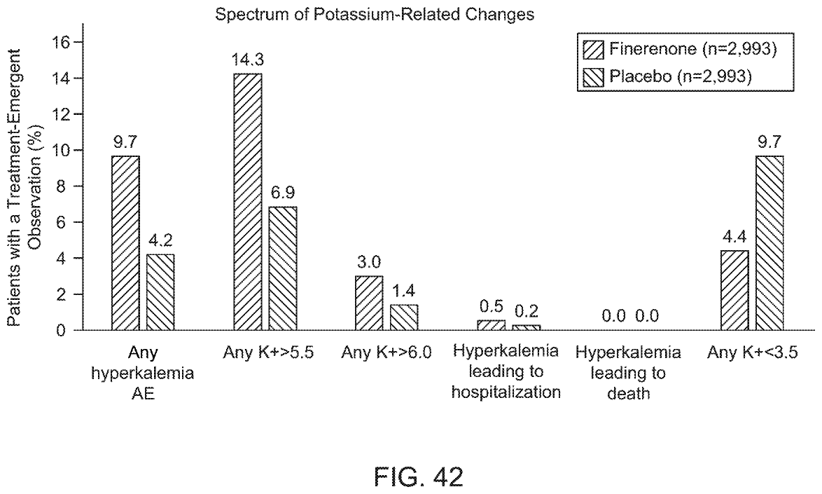

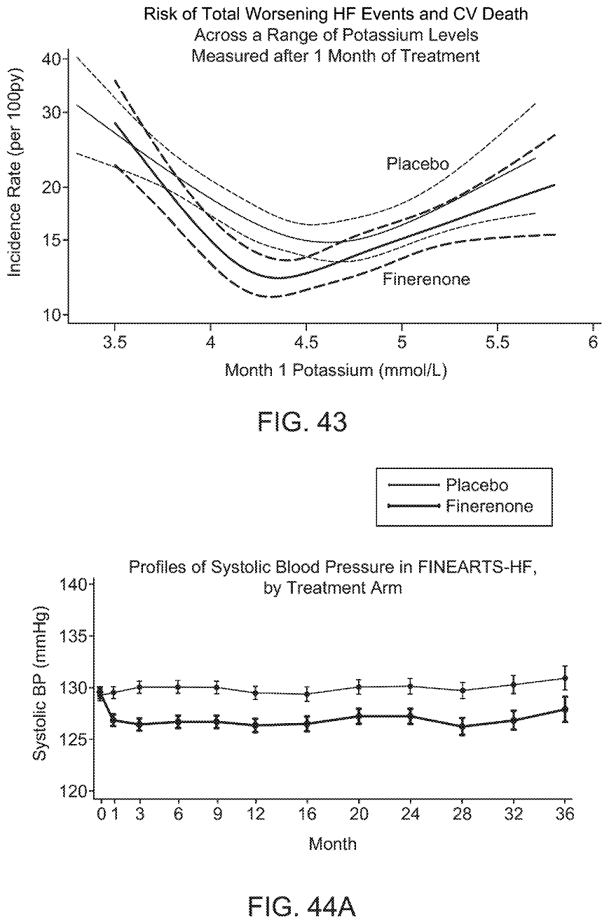

Among 6,001 randomized participants, mean baseline potassium levels were 4.4±0.5 mmol/L. At one month, the effect of finerenone on potassium was +0.19 (0.17-0.21) mmol/L, a difference that persisted during the trial (FIG. 41). Regardless of treatment assignment, patients who developed any potassium level >5.5 mmol/L in follow-up were more likely to be men, with histories of diabetes and recent worsening HF events, worse baseline kidney function and higher baseline potassium levels. Finerenone increased the risks of any potassium level >5.5 mmol/L (14.3% vs. 6.9%) and decreased the risks for hypokalemia (4.4% vs. 9.7%). Hyperkalemia-related hospitalizations were infrequent, and there were no hyperkalemia-related deaths in either arm (FIG. 42). During a median follow-up of 2.6 years, a U-shaped association was observed between potassium levels measured after one month of treatment and subsequent risks of the primary outcome, such that for any given level of potassium, risks were generally lower in patients treated with finerenone compared to patients treated with placebo (FIG. 43).

In patients with HFmrEF/HFpEF, finerenone resulted in early modest increases in potassium levels. However, the clinical benefit of finerenone was maintained even in the setting of moderate hyperkalemia.

### Example 21—Blood Pressure Effects of Finerenone in Heart Failure with Mildly Reduced or Preserved Ejection Fraction: A Prespecified Analysis of the FINEARTS-HF Trial

Blood pressure (BP) optimization is a guideline-recommended priority of heart failure (HF) care. The nonsteroidal mineralocorticoid receptor antagonist finerenone was recently shown to improve clinical outcomes in individuals with HF and mildly reduced or preserved ejection fraction (HFmrEF/HFpEF), but blood pressure effects of finerenone in this population have not been rigorously evaluated.

To evaluate 1) the effects of finerenone on systolic BP overall and in individuals with apparent treatment-resistant hypertension (aTRH); and 2) the relationship between the effects of finerenone on SBP and clinical outcomes.

In this prespecified analysis of the FINEARTS-HF trial, participants were categorized according to baseline BP category: aTRH (BP ≥140/90 mm Hg [≥130/80 mm Hg if diabetes] despite treatment with 3 antihypertensive drugs including a diuretic), non-resistant hypertension (BP above threshold but not meeting aTRH criteria), and controlled BP (BP under threshold). Treatment effects on systolic BP were evaluated overall and by BP category using linear regression with adjustment for baseline BP. To determine whether the BP effects of finerenone accounted for its benefits on the primary outcome of cardiovascular death and total worsening HF events, treatment effect rate ratios were estimated with and without adjustment for change in systolic BP between baseline and one month.

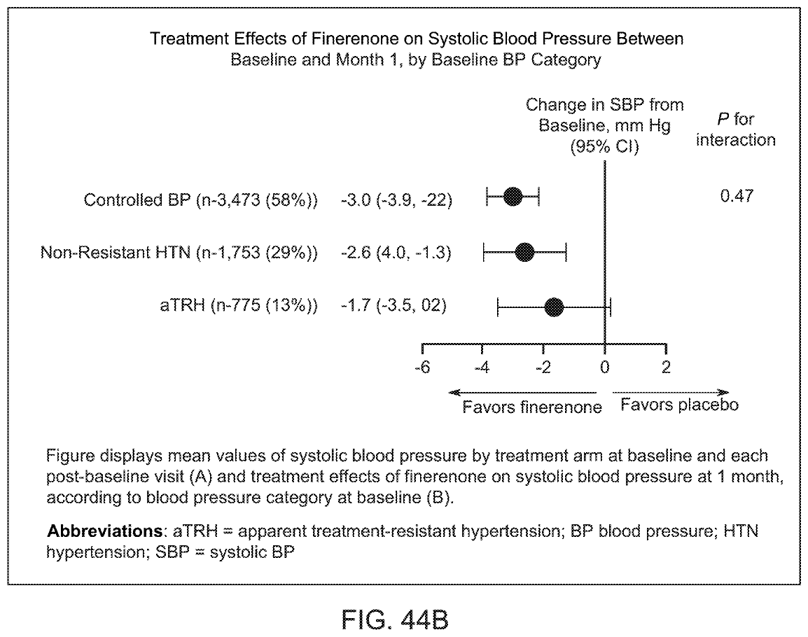

Among 6,001 participants, 3,473 (57.9%) had controlled blood pressure, 1,753 (29.2%) had non-resistant hypertension, and 775 (12.9%) had aTRH at baseline. In the overall FINEARTS-HF population, participants treated with finerenone experienced early and sustained reductions in systolic BP compared with placebo (FIG. 44A), with most treatment effects observed by one month (mean difference,-2.8 mm Hg [95% CI, −3.4 to −2.1]; P<0.001). Effects of finerenone on the primary outcome (Pinteraction=0.32) and BP lowering (Pinteraction=0.47) were similar across BP categories (FIG. 44B). Moreover, the effect of finerenone on cardiovascular death and total worsening HF events (rate ratio, 0.84 [95% CI, 0.74 to 0.95]; P=0.007) was not attenuated after adjustment for BP changes between baseline and 1 month (rate ratio, 0.84 [95% CI, 0.74 to 0.96]; P=0.009).

Finerenone resulted in early and sustained reductions in blood pressure in patients with HFmrEF/HFpEF, but these effects did not explain its benefits on clinical outcomes in this population.

### Example 22—Effect of Finerenone on Outpatient Worsening Heart Failure Events in Patients with Mildly Reduced or Preserved Ejection Fraction

Outpatient worsening heart failure (HF) events, including oral diuretic intensifications, are common and associated with poor subsequent prognosis. The effect of the non-steroidal mineralocorticoid antagonist finerenone on outpatient worsening HF events in patients with mildly reduced or preserved ejection fraction is unknown.

The FINEARTS-HF trial randomized 6,001 patients with HF and EF ≥40% to finerenone versus placebo. Primary outcome events (cardiovascular [CV] death, HF hospitalization, and outpatient urgent HF visit requiring intravenous diuretic therapy) were centrally adjudicated. In this pre-specified analysis, we defined outpatient oral diuretic intensification events as initiations of loop or thiazide diuretic, or increases in loop diuretic dosage. We evaluated the risk of all-cause death following each type of worsening HF event (HF hospitalization, urgent HF visit, or outpatient oral diuretic intensification) and the effect of finerenone on outpatient oral diuretic intensification alone or as part of an extended composite outcome with primary outcome events.

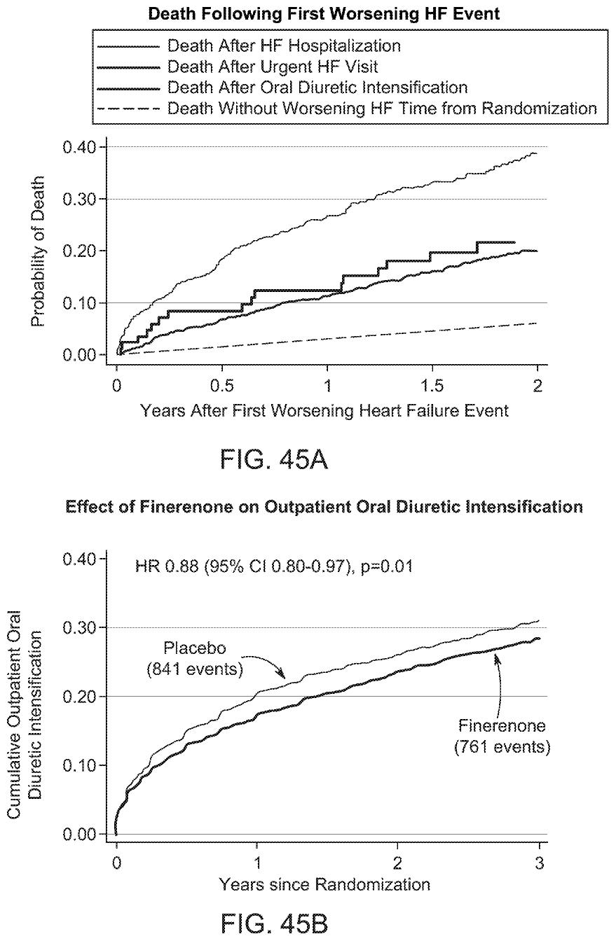

Worsening HF events occurred in 2,008 patients (33%). The first event was HF hospitalization in 657 (11%), urgent HF visit in 86 (1%), and outpatient oral diuretic intensification in 1,265 (21%). Increases in loop diuretic dose were the most common diuretic intensification (n=985, 78%). Rates of all-cause death were higher following worsening HF events—27.9 (95% CI 24.4-31.8) per 100 patient-years (py) after HF hospitalization, 13.2 (95% CI 8.4-20.7) per 100 py after urgent HF visit, and 11.5 (95% CI 10.2-13.0) per 100 py after outpatient oral diuretic intensification—compared to 4.6 (95% CI 4.2-5.0) per 100 py for patients who did not experience worsening HF (FIG. 45A). Addition of outpatient oral diuretic intensification to the primary outcome increased the number of patients experiencing an event from 1,343 to 2,246. Finerenone reduced the risk of outpatient oral diuretic intensification alone (hazard ratio [HR] 0.88 [95% CI 0.80-0.97], p=0.01) (FIG. 45B) and a composite outcome that further included CV death, HF hospitalization, and urgent HF visit (HR 0.85 [95% CI 0.78-0.93], p<0.001).

Outpatient worsening HF events are common, associated with poor prognosis, and were reduced by finerenone in patients with HF with mildly reduced or preserved ejection fraction.

### Example 23—Finerenone in Heart Failure with Improved Ejection Fraction

Patients with chronic heart failure and EF <40% who have improvement in ejection fraction to ≥40% (HFimpEF) may face residual risks similar to those observed in patients with left ventricular ejection fraction (LVEF) consistently above 40%. However, these patients remain understudied as they have commonly been excluded from HF clinical trials with mildly reduced and preserved EF. We assessed baseline characteristics, event rates, and treatment response to finerenone in participants with HFimpEF enrolled in the FINEARTS-HF trial.

### Methods

FINEARTS-HF enrolled patients with HF and LVEF ≥40% to finerenone or placebo and allowed enrollment of those with LVEF previously <40% at any point. The primary endpoint was the composite of cardiovascular (CV) death and total (first and recurrent) worsening HF events.

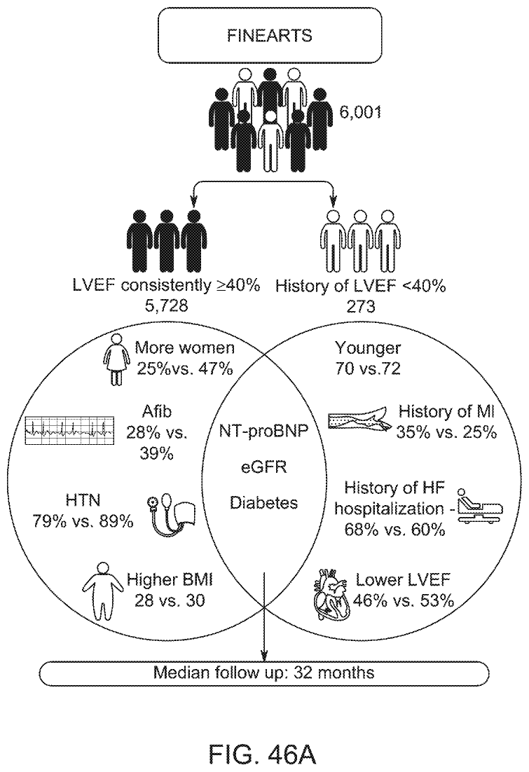

Of the 6,001 participants of FINEARTS-HF, 273 (5%) had a prior LVEF <40%. In these patients, the median recorded prior LVEF was 35% [30, 37] (median difference between prior LVEF and LVEF at enrollment of 12% [8, 17]). Compared to participants with LVEF consistently >40%, participants with history of LVEF <40% were younger with more history of myocardial infarction and had lower LVEF at enrollment. Both groups had similar eGFR and NT-proBNP levels (FIGS. 46A-46B). Over a median follow-up of 2.6 years, those with history of LVEF <40% experienced higher risk of the primary outcome (RR 1.33, 95% CI 1.01, 1.75, p=0.043) and total worsening HF events (RR 1.39, 95% CI 1.02, 1.90, p=0.035) but similar rates of CV death (HR 1.09, 95% CI 0.72, 1.64, p=0.69) than those with LVEF consistently ≥40%. Treatment effect of finerenone on the primary outcome was consistent in those with history of LVEF <40% and those with LVEF consistently ≥40% (Pinteraction 0.36) (FIGS. 46A-46B). There was no effect modification by history of LVEF<40% for any of the secondary outcomes. Absolute risk reduction was greater in those with HFimpEF (9.2 vs. 2.5 per 100 py). The safety profile of finerenone was similar in patients with and without previous LVEF<40%.

Patients with HFimpEF enrolled in FINEARTS-HF appear to face higher risks than those whose LVEF has been consistently ≥40%. The benefit and safety of finerenone was consistent irrespective of HFimpEF status.

FIGS. 46A-46B show baseline characteristics and treatment response of participants with history of LVEF <40% and those with LVEF consistently ≥40%.

### Example 24—Finerenone, Bilirubin, and Heart Failure with Mildly Reduced or Preserved Ejection Fraction: An Analysis from FINEARTS-HF

Elevated bilirubin levels are associated with worse outcomes in HF, possibly because it is a marker of heart congestion, and certain guideline-recommended therapies improve measures of liver function, including bilirubin. The effects of finerenone were examined, compared to placebo, on 1) clinical outcomes according to baseline bilirubin and 2) the change in bilirubin during follow-up.

FINEARTS-HF randomized 6,001 patients with HF and LVEF >40% to finerenone or placebo. Total bilirubin (mg/dL) was examined according to tertiles. The primary outcome was CV death and total worsening HF events.

Data on bilirubin were available in 5,873 patients (median 0.5 [IQR 0.4-0.8]). The effect of finerenone on the primary outcome did not vary by bilirubin tertile (T1, RR 0.94 [95% CI, 0.75-1.17]; T2, 0.83 [0.66-1.05]; T3, 0.77 [0.62-0.98]; Pinteraction=0.21). The beneficial effect of finerenone appeared to be greater in those with higher bilirubin when examined as a continuous variable (Pinteraction=0.09). A similar pattern was observed for total worsening HF events and improvement in the KCCQ-TSS. Consistent effects were observed for CV and all-cause death. Treatment with finerenone led to a sustained reduction in bilirubin early after randomization.

The beneficial effects of finerenone were consistent, irrespective of bilirubin level at baseline, possibly with a greater effect on worsening HF events with higher bilirubin. In addition, finerenone led to a reduction in bilirubin.

TABLE 30
Effects of finerenone compared with placebo on outcomes
Tertiles of bilirubin

| | Tertile 1 | | Tertile 2 | | Tertile 3 | | |
|---|---|---|---|---|---|---|---|
| | ≤0.4 mg/dL | | 0.5-0.6 mg/dL | | ≥0.7 mg/dL | | |
| | N = 1,923 | | N = 1,932 | | N = 2,018 | | P-value |
| | Finerenone | Placebo | Finerenone | Placebo | Finerenone | Placebo | for |
| | N = 957 | N = 966 | N = 970 | N = 962 | N = 1,009 | N = 1,009 | interaction |
| CV death and total worsening HF events | | | | | | | 0.21 |
| Event rate per 100 PY | 15.5 | 16.5 | 12.9 | 16.1 | 16.2 | 20.2 | |
| Rate ratio (95% CI)* | 0.94 (0.75-1.17) | | 0.83 (0.66-1.05) | | 0.77 (0.62-0.97) | | |
| Total worsening HF events | | | | | | | 0.23 |
| Event rate per 100 PY | 12.4 | 13.2 | 9.7 | 12.9 | 12.6 | 16.0 | |
| Rate ratio (95% CI)* | 0.94 (0.73-1.21) | | 0.79 (0.60-1.03) | | 0.76 (0.59-0.97) | | |
| CV death | | | | | | | 0.52 |
| Event rate per 100 PY | 3.1 | 3.2 | 3.2 | 3.3 | 3.6 | 4.2 | |
| Hazard ratio (95% CI)* | 0.95 (0.68-1.31) | | 0.97 (0.71-1.34) | | 0.85 (0.64-1.13) | | |
| All-cause death | | | | | | | 0.63 |
| Event rate per 100 PY | 6.8 | 7.4 | 6.4 | 6.4 | 6.6 | 7.6 | |
| Hazard ratio (95% CI)* | 0.92 (0.74-1.14) | | 1.00 (0.80-1.26) | | 0.85 (0.69-1.05) | | |
| Change in KCCQ-TSS from baseline to 12 months | | | | | | | 0.10 |
| Mean change (SD) | 8.1 (20.8) | 8.0 (21.2) | 10.6 (21.8) | 8.0 (21.1) | 5.4 (24.6) | 7.8 (24.0) | |
| Difference in mean | 1.13 (−0.62 to 2.89) | | 0.69 (−0.92 to 2.30) | | 3.15 (1.52 to 4.77) | | |
| (95% CI)** | | | | | | | |

*Models were adjusted for geographic region and baseline LVEF.
**Models were adjusted for baseline value, geographic region, and baseline LVEF.

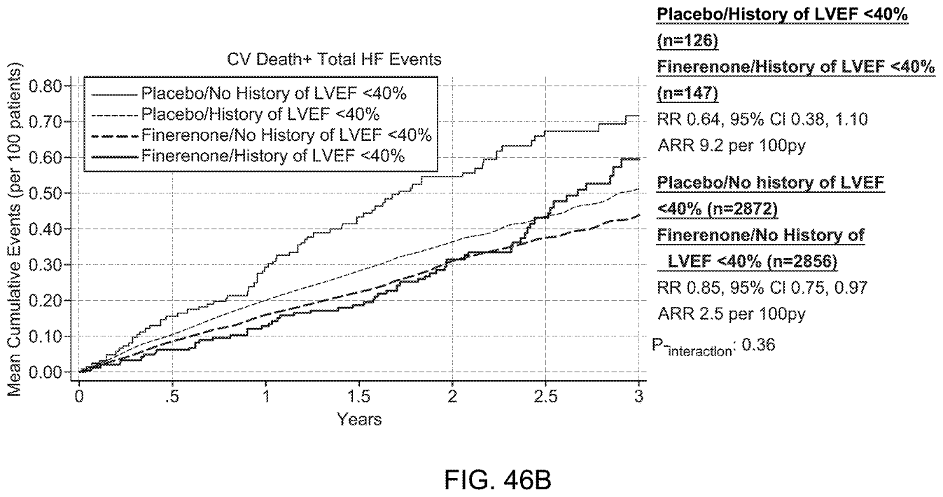

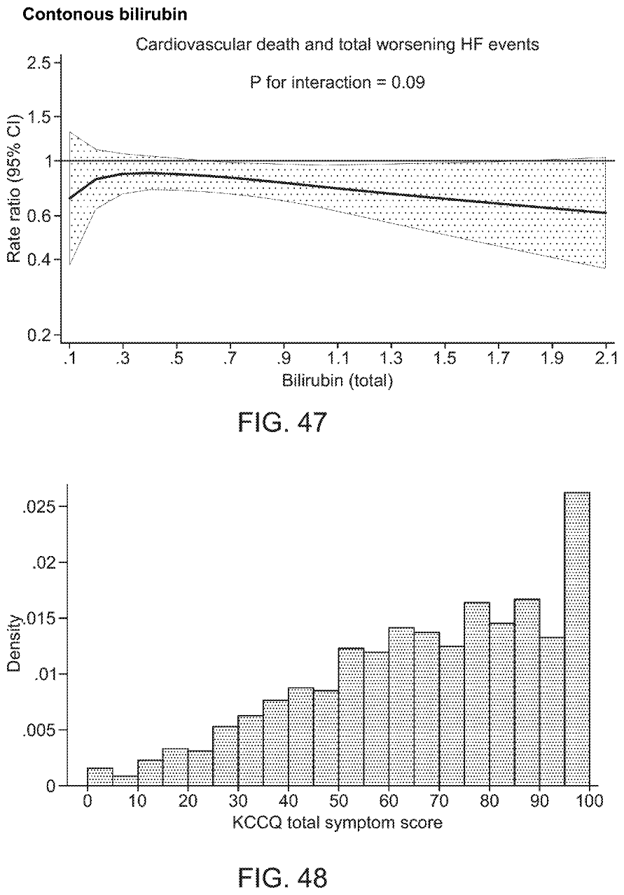

See also FIG. 47.

### Example 25—Effect Finerenone on the KCCQ with patients with HFmrEF/HFpEF

Patients with heart failure (HF) are limited by symptoms and have impaired quality of life. The Kansas City Cardiomyopathy Questionnaire (KCCQ) is a patient-reported outcome measure that enables evaluation of the impact of HF and the effect of new therapies on health status in patients with HF.

This pre-specified analysis of FINEARTS-HF (Finerenone trial to investigate efficacy and safety superior to placebo in patients with heart failure) assessed the efficacy and safety of finerenone according to baseline KCCQ Total Symptom Score (KCCQ-TSS) and the effect of finerenone on KCCQ-TSS.

FINEARTS-HF tested the efficacy of the non-steroidal mineralocorticoid receptor antagonist (MRA) finerenone, compared to placebo, in patients with HFmrEF/HFpEF. The primary endpoint was the composite of cardiovascular death and total worsening HF events. The KCCQ was completed by patients at randomization and at 6, 9, and 12 Months after randomization. Change in KCCQ-TSS was a key secondary endpoint. Patients were stratified by KCCQ-TSS tertiles at baseline. The association between KCCQ tertile and clinical outcomes was evaluated using semiparametric proportional-rates models for total events and Cox models for time-to-first-event data and the effects of finerenone versus placebo on the primary endpoint were assessed across tertiles of KCCQ-TSS.

Of the 6,001 participants in FINEARTS-HF, 5,986 (99.8%) had baseline KCCQ-TSS recorded (median score 69.8 out of a possible 100; higher score=better health status). Lower (worse) KCCQ-TSS was associated with a higher risk of the primary endpoint. Finerenone, compared to placebo, reduced the risk of the primary endpoint across the range of KCCQ-TSS: tertile 1 (score 0 to <57): rate ratio (RR) 0.82 (95% CI, 0.68-1.00); tertile 2 (57 to <81): 0.88 (0.70-1.11); tertile 3 (81 to 100): 0.88 (0.69-1.14); Pinteraction=0.89. Compared to placebo, finerenone significantly improved KCCQ-TSS from baseline with a mean difference at 12 months of 1.62 (95% CI 0.69-2.56) points, p<0.001. Numerically fewer finerenone-treated patients experienced clinically meaningful deterioration, and more had improvements in KCCQ-TSS.

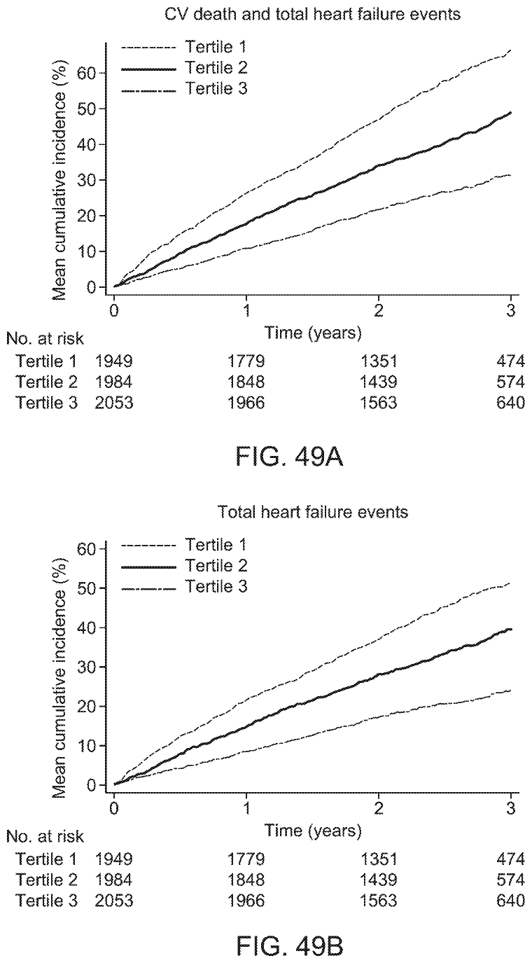

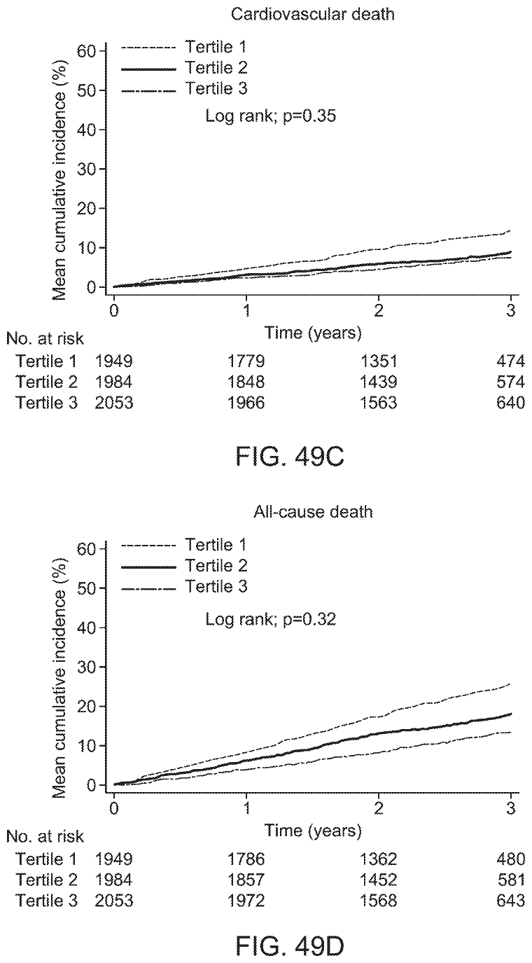

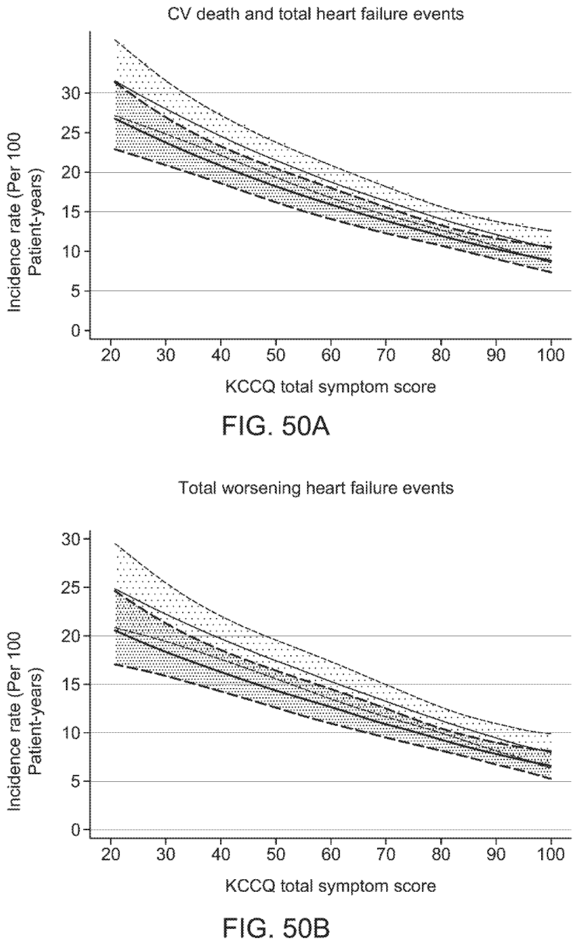

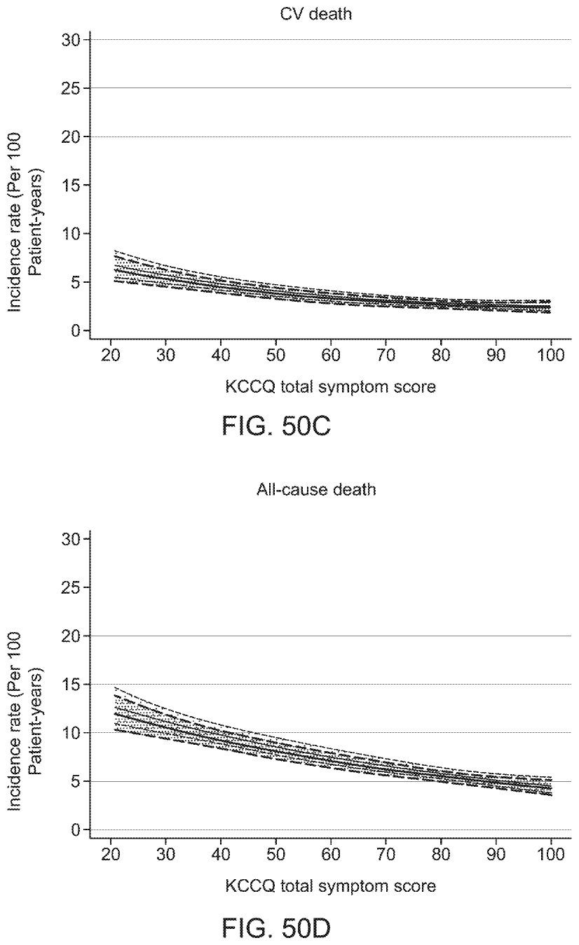

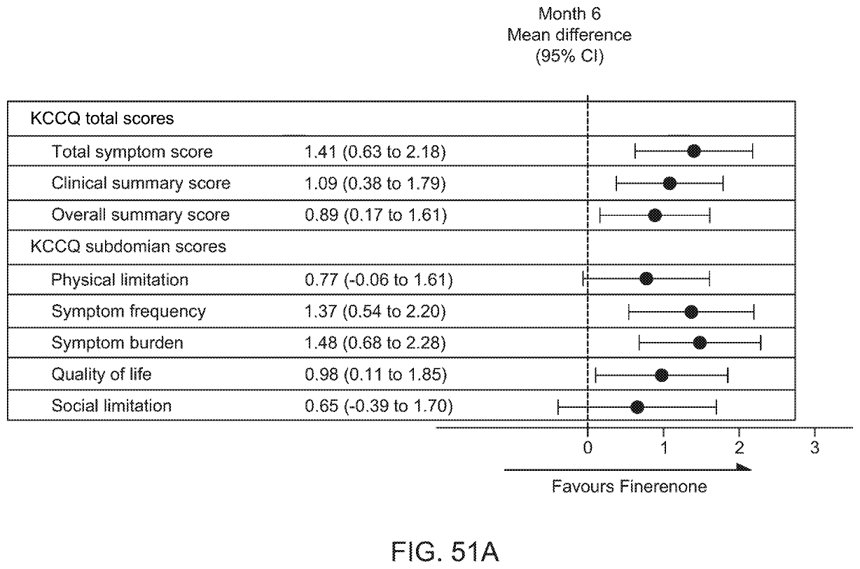

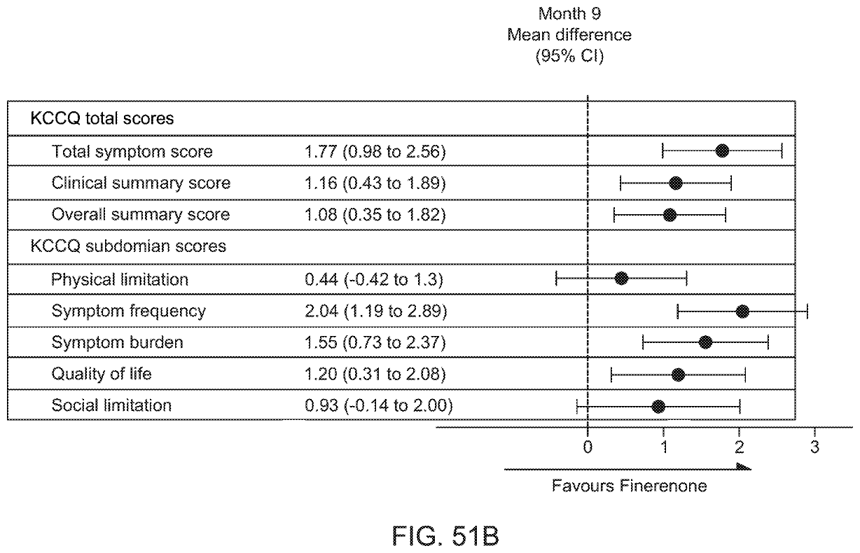

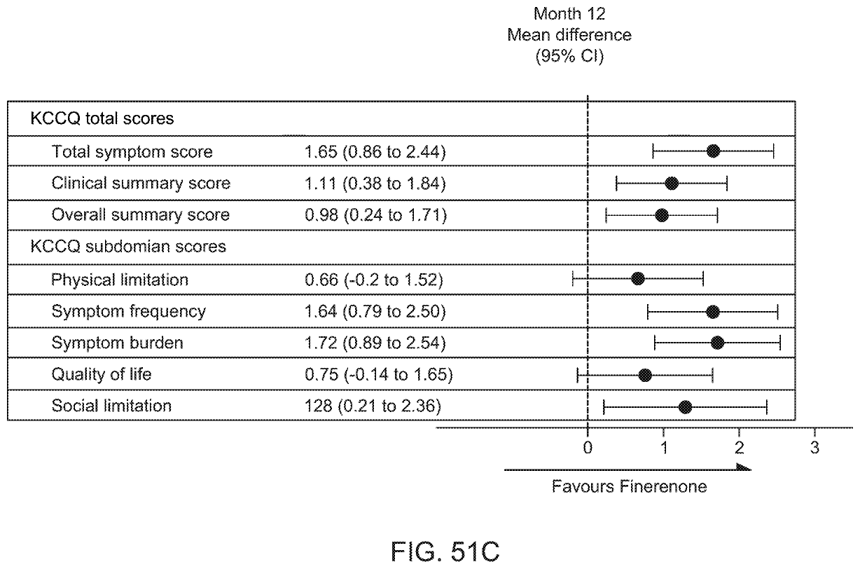

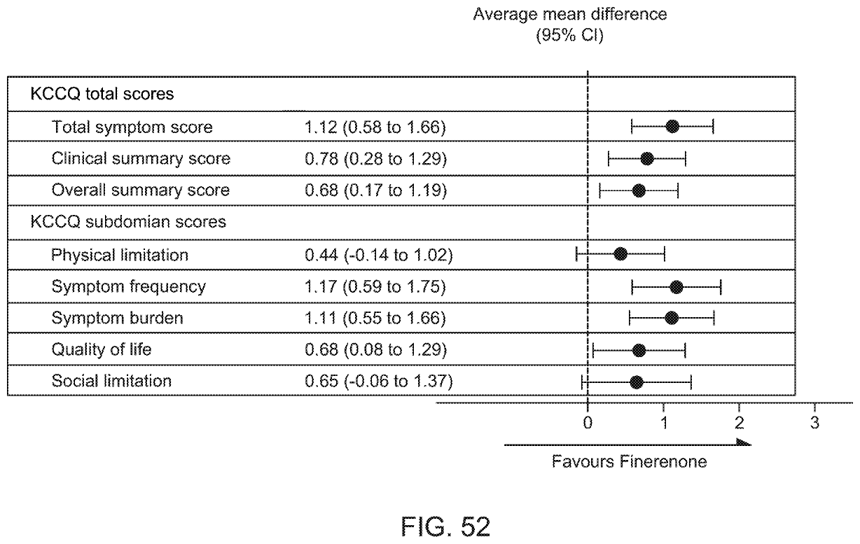

Finerenone significantly reduced HF events and improved health status in HFmrEF/HFpEF across the spectrum of KCCQ-TSS at baseline. In conclusion, the FINEARTS-HF trial demonstrates that finerenone, when added to usual therapy, significantly improves health status, particularly in those with a higher symptom burden, and reduces the risk of adverse clinical outcomes in patients with HFmrEF/HFpEF regardless of health status impairment at baseline. Collectively, these findings support the use of finerenone as an efficacious therapeutic option in this population.See FIG. 48, FIGS. 49A-49D, FIGS. 50A-50D, FIG. 51, and FIG. 52.

TABLE 31
Patients completed KCCQ total and domain
scores at baseline and during follow-up

| | Baseline | 6 months | 9 months | 12 months |
|---|---|---|---|---|
| N | 6,001 | 6,001 | 6,001 | 6,001 |
| Total scores | | | | |
| Total symptom score | 5,986 (99.8) | 5,356 (89.3) | 5,036 (83.9) | 5,004 (83.4) |
| Clinical summary score | 5,987 (99.8) | 5,359 (89.3) | 5,037 (83.9) | 5,006 (83.4) |
| Overall summary score | 5,987 (99.8) | 5,359 (89.3) | 5,037 (83.9) | 5,006 (83.4) |
| Domain scores* | | | | |
| Physical limitation | 5,892 (98.2) | 5,215 (86.9) | 4,872 (81.2) | 4,848 (80.8) |
| Symptom frequency | 5,986 (99.8) | 5,356 (89.3) | 5,035 (83.9) | 5,003 (83.4) |
| Symptom burden | 5,985 (99.7) | 5,355 (89.2) | 5,035 (83.9) | 5,003 (83.4) |
| Quality of life | 5,986 (99.8) | 5,355 (89.2) | 5,036 (83.9) | 5,004 (83.4) |
| Social limitation | 5,574 (92.9) | 4,772 (79.5) | 4,460 (74.3) | 4,465 (74.4) |

Data are presented as N (%).
*Domain scores used to create summary scores.
KCCQ, Kansas City Cardiomyopathy Questionnaire.

TABLE 32
Clinical outcomes according to baseline KCCQ-TSS category divided by tertile

| | Tertile 1: 0 to <57 | Tertile 2: 57 to <81 | Tertile 3: 81 to 100 |
|---|---|---|---|
| N | 1,949 | 1,984 | 2,053 |
| CV death and total | | | |
| worsening HF events | | | |
| No. of events (%) | 1,031 (568, 29.1%) | 786 (441, 22.2%) | 540 (329, 16.0%) |
| Rate per 100 patient- | 22.8 (20.6, 25.1) | 16.3 (14.5, 18.3) | 10.5 (9.2, 11.9) |
| years (95% CI) | | | |
| RR (95% CI)* | Ref. | 0.69 (0.59, 0.80) | 0.40 (0.34, 0.47) |
| Adjusted RR (95% CI)** | Ref. | 0.85 (0.72, 0.99) | 0.59 (0.49, 0.72) |
| Total worsening HF | | | |
| events | | | |
| No. of events (%) | 805 (446, 22.9%) | 639 (352, 17.7%) | 414 (249, 12.1%) |
| Rate per 100 patient- | 17.8 (15.9, 19.9) | 13.3 (11.7, 15.1) | 8.0 (7.0, 9.3) |
| years (95% CI) | | | |
| RR (95% CI)* | ref | 0.71 (0.60, 0.85) | 0.38 (0.31, 0.47) |
| Adjusted RR (95% CI)** | ref | 0.88 (0.74, 1.06) | 0.57 (0.45, 0.72) |
| Total HF | | | |
| hospitalizations | | | |
| No. of events (%) | 712 (418, 21.4%) | 545 (322, 16.2%) | 356 (219, 10.7%) |
| Rate per 100 patient- | 15.7 (14.1, 17.6) | 11.3 (9.9, 12.9) | 6.9 (5.9, 8.1) |
| years (95% CI) | | | |
| RR (95% CI)* | ref | 0.69 (0.58, 0.82) | 0.37 (0.30, 0.46) |
| Adjusted RR (95% CI)** | ref | 0.86 (0.72, 1.03) | 0.56 (0.44, 0.70) |
| Total urgent HF visits | | | |
| No. of events (%) | 93 (70, 3.6%) | 94 (67, 3.4%) | 58 (49, 2.4%) |
| Rate per 100 patient- | 2.1 (1.6, 2.7) | 1.9 (1.5, 2.6) | 1.1 (0.8, 1.5) |
| years (95% CI) | | | |
| RR (95% CI)* | ref | 0.88 (0.61, 1.27) | 0.47 (0.31, 0.71) |
| Adjusted RR (95% CI)** | ref | 1.02 (0.69, 1.50) | 0.69 (0.42, 1.11) |
| CV death or worsening | | | |
| HF | | | |
| No. of events (%) | 568 (568, 29.1%) | 441 (441, 22.2%) | 329 (329, 16%) |
| Rate per 100 patient- | 14.4 (13.2, 15.6) | 10.0 (9.1, 11.0) | 6.8 (6.1, 7.6) |
| years (95% CI) | | | |
| HR (95% CI)* | ref | 0.68 (0.60, 0.77) | 0.41 (0.36, 0.48) |
| Adjusted HR (95% CI)** | ref | 0.83 (0.72, 0.94) | 0.59 (0.50, 0.69) |
| Worsening HF | | | |
| No. of events (%) | 446 (446, 22.9%) | 352 (352, 17.7%) | 249 (249, 12.1%) |
| Rate per 100 patient- | 11.3 (10.2, 12.4) | 8.0 (7.2, 8.9) | 5.1 (4.5, 5.8) |
| years (95% CI) | | | |
| HR (95% CI)* | ref | 0.68 (0.59, 0.78) | 0.39 (0.33, 0.46) |
| Adjusted HR (95% CI)** | ref | 0.85 (0.73, 0.99) | 0.56 (0.47, 0.68) |
| CV death | | | |
| No. of events (%) | 226 (226, 11.6%) | 147 (147, 7.4%) | 128 (128, 6.2%) |
| Rate per 100 patient- | 5.0 (4.4, 5.7) | 3.0 (2.6, 3.6) | 2.5 (2.1, 3.0) |
| years (95% CI) | | | |
| HR (95% CI)* | ref | 0.61 (0.49, 0.75) | 0.48 (0.38, 0.60) |
| Adjusted HR (95% CI)** | ref | 0.71 (0.57, 0.89) | 0.68 (0.53, 0.89) |
| CV death or first HF | | | |
| hospitalization | | | |
| No. of events (%) | 541 (541, 27.8%) | 415 (415, 20.9%) | 302 (302, 14.7%) |
| Rate per 100 patient- | 13.5 (12.4, 14.7) | 9.3 (8.5, 10.3) | 6.2 (5.5, 6.9) |
| years (95% CI) | | | |
| HR (95% CI)* | ref | 0.67 (0.59, 0.76) | 0.40 (0.34, 0.47) |
| Adjusted HR (95% CI)** | ref | 0.82 (0.72, 0.94) | 0.57 (0.48, 0.67) |
| First HF hospitalization | | | |
| No. of events (%) | 418 (418, 21.4%) | 322 (322, 16.2%) | 219 (219, 10.7%) |
| Rate per 100 patient- | 10.4 (9.4, 11.5) | 7.2 (6.5, 8.1) | 4.5 (3.9, 5.1) |
| years (95% CI) | | | |
| HR (95% CI)* | ref | 0.67 (0.57, 0.77) | 0.36 (0.30, 0.43) |
| Adjusted HR (95% CI)** | ref | 0.84 (0.72, 0.98) | 0.53 (0.44, 0.64) |
| All cause death | | | |
| No. of events (%) | 444 (444, 22.8%) | 324 (324, 16.3%) | 241 (241, 11.7%) |
| Rate per 100 patient- | 9.8 (8.9, 10.7) | 6.7 (6.0, 7.4) | 4.7 (4.1, 5.3) |
| years (95% CI) | | | |
| HR (95% CI)* | ref | 0.67 (0.58, 0.77) | 0.44 (0.37, 0.52) |
| Adjusted HR (95% CI)** | ref | 0.78 (0.67, 0.91) | 0.62 (0.52, 0.75) |

Data are presented as the number of events (number of patients who had an event, %) for recurrent event, and number of patient (%) for time-to-first event.
*Stratified by geographic region and baseline LVEF (<60%, ≥60%).
**Adjusted for age, sex, heart rate, systolic blood pressure, body mass index, prior hospitalization for HF, NYHA functional class III/IV, left ventricular ejection fraction, estimated glomerular filtration rate, NT-proBNP (log-transformed), atrial fibrillation, myocardial infarction, and diabetes mellitus.

TABLE 33
Safety outcomes according to baseline
KCCQ-TSS category divided by tertile

| | Tertile 1: | Tertile 2: | Tertile 3: |
|---|---|---|---|
| | 0 to <57 | 57 to <81 | 81 to 100 |
| N | 1,949 | 1,984 | 2,053 |
| Systolic blood | 248 (13.2) | 257 (13.4) | 391 (19.6) |
| pressure <100 mmHg | | | |
| Creatinine ≥3 mg/dL | 42 (2.3) | 27 (1.4) | 22 (1.1) |
| Creatinine ≥2.5 mg/dL | 97 (5.2) | 67 (3.5) | 66 (3.3) |
| Potassium >6 mmol/L | 39 (2.1) | 51 (2.7) | 36 (1.8) |
| Potassium >5.5 mmol/L | 198 (10.6) | 214 (11.2) | 198 (9.9) |
| Potassium <3.5 mmol/L | 159 (8.5) | 101 (5.3) | 147 (7.4) |

Values are N (%) unless otherwise stated. Safety analyses were carried out in patients who had undergone enrolment and received at least one dose of the randomized treatment (a total of 15 randomized patients were excluded from the safety analysis). Safety events were considered treatment-emergent if they occurred between the day of treatment initiation up to and including three days after treatment discontinuation.

### Example 26—Efficacy and Safety of Finerenone in these Patients According to Age

Finerenone improves outcomes in patients with HF and mildly reduced or preserved ejection fraction (HFmrHF/HFpEF). It is important to understand the efficacy and safety of finerenone in these patients according to age.

Methods: The aim of this analysis was to evaluate the interaction between age and the efficacy and safety of finerenone in the FINEARTS-HF trial (Finerenone trial to investigate efficacy and safety compared to placebo in patients with heart failure). A total of 6,001 patients aged 40-97 years were stratified by quartile (Q 1-4) of baseline age: Q1 40-66 years (n=1,581), Q2 67-73 years (n=1,587), Q 3 74-79 years (n=1,421), and Q4≥80 years (n=1,412). This study evaluated the impact of age on the efficacy of finerenone with respect to the primary composite outcome of cardiovascular death and the total (first and recurrent) HF events, including HF hospitalization or urgent HF event, along with secondary efficacy and safety outcomes.

Results: The incidence of primary outcome increased with age. Finerenone reduced the risk of the primary outcome consistently across all age categories: RR (95% CI) Q1 0.70 (0.53-0.92), Q2 0.83 (0.64-1.07), Q3 0.98 (0.76-1.26), and Q4 0.85 (0.67-1.07); p for interaction=0.27. Similarly consistent effects were observed for the components of the primary outcome, and all-cause death. The mean increase in Kansas City Cardiomyopathy Questionnaire-total symptom score (KCCQ-TSS) from baseline to 12 months was greater with finerenone than placebo, with a consistent effect across all age categories: mean placebo-corrected change (95% CI) Q1 2.87 (1.09-4.66), Q2 1.24 (−0.59-3.07), Q3 0.94 (−0.98-2.86), and Q4 1.24 (−0.90-3.38); P for interaction=0.50. Adverse events of interest were similar across all age categories.

Conclusions: In the FINEARTS-HF trial, finerenone reduced the primary outcome, components of the primary outcome, and all-cause death, and improved symptoms across a wide age spectrum. Finerenone reduced the risk of HF events, cardiovascular death, and all-cause mortality, while also improving health-related quality of life and HF symptoms in patients with HFmrEF or HFpEF across the age spectrum. In addition, finerenone was safe and well-tolerated, irrespective of age.

### Example 27—Effects of the Non-Steroidal MRA Finerenone with and without Concomitant SGLT2 Inhibitor Use in Heart Failure

Patients with heart failure (HF) with mildly reduced or preserved ejection fraction face heightened long-term risks of morbidity and mortality. The sodium glucose-co-transporter-2 inhibitors (SGLT2i) and the non-steroidal mineralocorticoid receptor antagonist (MRA) finerenone have both been shown to reduce the risk of cardiovascular events in this population, but the effects of their combined use are not known.

FINEARTS-HF was a randomized, double-blind, placebo-controlled trial of finerenone in patients with HF and left ventricular ejection fraction (LVEF)>40%. Baseline SGLT2i use was a prespecified subgroup. The primary outcome was a composite of total (first and recurrent) worsening HF events and cardiovascular death. We first assessed for evidence of treatment heterogeneity based on baseline SGLT2i use. We further examined SGLT2i uptake during the trial and evaluated the treatment effects of finerenone accounting for baseline and during trial use of SGLT2i in time-varying analyses.

Among 6,001 participants, 817 (13.6%) were treated with an SGLT2i at baseline. During 2.6-years median follow-up, treatment with finerenone similarly reduced the risk of the primary outcome in participants treated with an SGLT2i (rate ratio 0.83; 95% confidence interval 0.60 to 1.16) and without an SGLT2i at baseline (rate ratio 0.85; 95% confidence interval 0.74 to 0.98); Pinteraction=0.76. In follow-up, 980 participants initiated SGLT2i, which was less frequent in the finerenone arm compared with placebo (17.7% vs. 20.1%; hazard ratio 0.86; confidence interval 0.76 to 0.97). Time-updated analyses accounting for baseline and subsequent use of SGLT2i did not meaningfully alter the treatment effects of finerenone on the primary endpoint.

The treatment benefits of the non-steroidal MRA finerenone were observed irrespective of concomitant use of an SGLT2i. These data suggest that the combination use of SGLT2i and a non-steroidal MRA may provide additive protection against cardiovascular events in patients with HF with mildly reduced or preserved ejection fraction.

In summary, this prespecified analysis of FINEARTS-HF found that the non-steroidal MRA finerenone reduced cardiovascular death and total HF events irrespective of baseline or subsequent SGLT2i use and supports the complementary roles of the non-steroidal MRA finerenone and SGLT2i in the management of patients with HFmrEF or HFpEF.

TABLE 34
Baseline Characteristics by Concomitant SGLT2 Inhibitor Use

| | No SGLT2i Use | SGLT2i Use |
|---|---|---|
| | n = 5,184 | n = 817 |
| Age | 72.1 ± 9.5 | 71.4 ± 10.5 |
| Women | 2,412 (46.5%) | 320 (39.2%) |
| Race | | |
| Asian | 803 (15.5%) | 193 (23.6%) |
| Black | 77 (1.5%) | 11 (1.3%) |
| Other | 138 (2.7%) | 44 (5.4%) |
| White | 4,166 (80.4%) | 569 (69.6%) |
| Region | | |
| Asia | 793 (15.3%) | 190 (23.3%) |
| Eastern Europe | 2,464 (47.5%) | 186 (22.8%) |
| Latin America | 530 (10.2%) | 111 (13.6%) |
| North America | 388 (7.5%) | 83 (10.2%) |
| Western Europe, Oceania and Others | 1,009 (19.5%) | 247 (30.2%) |
| Any prior HF Hospitalization | 3,036 (58.6%) | 583 (71.4%) |
| Recency of Heart Failure Event | | |
| ≤7 days from randomization | 1,031 (19.9%) | 188 (23.0%) |
| >7 days to ≤3 months | 1,657 (32.0%) | 371 (45.4%) |
| >3 months or no index HF event | 2,496 (48.1%) | 258 (31.6%) |
| Systolic Blood Pressure (mmHg) | 129.9 ± 15.1 | 126.4 ± 16.5 |
| Body Mass Index (kg/m²) | 29.9 ± 6.1 | 30.0 ± 6.3 |
| Serum Creatinine (mg/dL) | 1.1 ± 0.4 | 1.2 ± 0.4 |
| eGFR (mL/min/1.73 m²) | 62.5 ± 19.5 | 59.6 ± 20.8 |
| eGFR <60 mL/min/1.73 m² | 2,439 (47.0%) | 449 (55.0%) |
| UACR (mg/g) | 17 [6-59] | 32 [11-129] |
| Potassium (mmol/L) | 4.4 ± 0.5 | 4.4 ± 0.5 |
| LVEF (%) | 52.7 ± 7.8 | 51.5 ± 7.8 |
| NT-proBNP (pg/mL) | 1,014 [438-1907] | 1,180 [527-2263] |
| NYHA class | | |
| NYHA Class II | 3,577 (69.0%) | 569 (69.6%) |
| NYHA ClassIII | 1,573 (30.3%) | 240 (29.4%) |
| NYHA Class IV | 33 (0.6%) | 8 (1.0%) |
| Hypertension | 4,593 (88.6%) | 732 (89.6%) |
| Diabetes Mellitus | 1,823 (35.2%) | 616 (75.4%) |
| Atrial Fibrillation on Electrocardiogram | 1,983 (38.3%) | 310 (37.9%) |
| Prior Stroke | 605 (11.7%) | 103 (12.6%) |
| Prior Myocardial Infarction | 1,314 (25.3%) | 227 (27.8%) |
| β-blocker | 4,405 (85.0%) | 690 (84.5%) |
| ACEI | 1,926 (37.2%) | 229 (28.0%) |
| ARB | 1,819 (35.1%) | 283 (34.6%) |
| ARNI | 350 (6.8%) | 163 (20.0%) |
| Calcium channel blockers | 1,710 (33.0%) | 258 (31.6%) |
| Loop diuretic | 4,490 (86.6%) | 749 (91.7%) |

Abbreviations:
ACEi = angiotensin converting enzyme inhibitor;
ARB = angiotensin receptor blocker;
ARNI = angiotensin receptor neprilysin inhibitor;
eGFR = estimated glomerular filtration;
LVEF = left ventricular ejection fraction;
NYHA = New York Heart Association;
SGLT2i = sodium glucose co-transporter-2 inhibitor;
UACR = urine albumin creatinine ratio

TABLE 35
Key Outcomes by Concomitant SGLT2 Inhibitor Use

| | | | Rate Ratio (RR) | |
|---|---|---|---|---|
| | | | or Hazard Ratio | |
| | No SGLT2i Use | | (HR) (95% CI) | |
| | (Reference) | SGLT2i Use | Relative to No | P- |
| | n = 5,184 | n = 817 | SGLT2i Use | value |
| CV death and total | 1,956 Events | 410 Events | RR 1.57 | <0.001 |
| worsening HF events | | | (1.30, 1.88) | |
| | [15.2/100 py] | [24.2/100 py] | | |
| Total worsening | 1,533 Events | 333 Events | RR 1.61 | <0.001 |
| HF events | | | (1.31, 1.97) | |
| | [11.9/100 py] | [19.7/100 py] | | |
| CV death | 425 (8.2%) | 77 (9.4%) | HR 1.40 | 0.007 |
| | | | (1.10, 1.79) | |
| | [3.3/100 py] | [4.6/100 py] | | |
| All-cause death | 876 (16.9%) | 137 (16.8%) | HR 1.20 | 0.050 |
| | | | (1.00, 1.43) | |
| | [6.8/100 py] | [8.0/100 py] | | |
| Renal composite | 112 (2.2%) | 18 (2.2%) | HR 1.31 | 0.284 |
| outcome | | | (0.80, 2.17) | |
| | [1.0/100 py] | [1.2/100 py] | | |
| CV death or first | 1,126 (21.7%) | 217 (26.6%) | HR 1.45 | <0.001 |
| worsening HF event | | | (1.25, 1.68) | |
| | [9.6/100 py] | [14.5/100 py] | | |
| CV death or first | 1,054 (20.3%) | 209 (25.6%) | HR 1.50 | <0.001 |
| HF hospitalization | | | (1.29, 1.74) | |
| | [8.9/100 py] | [13.8/100 py] | | |

Abbreviations:
CI = confidence interval;
CV = cardiovascular;
HF = heart failure;
HR = hazard ratio;
RR = rate ratio;
SGLT2i = sodium-glucose-co-transporter-2 inhibitor

TABLE 36
Treatment Effects of Finerenone by Concomitant SGLT2 Inhibitor Use

| | No SGLT2i Use | | SGLT2i Use | | |
|---|---|---|---|---|---|
| | Placebo | Finerenone | Placebo | Finerenone | |
| | n = 2,574 | n = 2,610 | n = 424 | n = 393 | P_{interaction} |
| CV death and total | | | | | |
| worsening HF events | | | | | |
| Events | 907 | 1049 | 176 | 234 | |
| Rate (per 100 py) | 14.0 | 16.5 | 21.8 | 26.5 | |
| RR (95% CI) | 0.85 (0.74, 0.98) | | 0.83 (0.60, 1.16) | | 0.76 |
| Total worsening HF | | | | | |
| events | | | | | |
| Events | 703 | 830 | 139 | 194 | |
| Rate (per 100 py) | 10.9 | 13.0 | 17.2 | 22.0 | |
| RR (95% CI) | 0.83 (0.71, 0.97) | | 0.80 (0.55, 1.15) | | 0.68 |
| CV death | | | | | |
| Events (%) | 205 (7.9%) | 220 (8.5%) | 37 (9.4%) | 40 (9.4%) | |
| Rate (per 100 py) | 3.2 | 3.5 | 4.6 | 4.5 | |
| HR (95% CI) | 0.92 (0.76, 1.11) | | 1.03 (0.65, 1.62) | | 0.73 |
| All-cause death | | | | | |
| Events (%) | 428 (16.4%) | 448 (17.4%) | 63 (16.0%) | 74 (17.5%) | |
| Rate (per 100 py) | 6.6 | 7.0 | 7.7 | 8.4 | |
| HR (95% CI) | 0.94 (0.82, 1.07) | | 0.90 (0.64, 1.27) | | 0.78 |
| Renal composite | | | | | |
| endpoint | | | | | |
| Events (%) | 67 (2.6%) | 45 (1.7%) | 8 (2.0%) | 10 (2.4%) | |
| Rate (per 100 py) | 1.2 | 0.8 | 1.1 | 1.3 | |
| HR (95% CI) | 1.43 (0.98, 2.08) | | 0.95 (0.37, 2.45) | | 0.37 |
| CV death or first | | | | | |
| worsening HF event | | | | | |
| Events (%) | 529 (20.3%) | 597 (23.2%) | 95 (24.2%) | 122 (28.8%) | |
| Rate (per 100 py) | 8.9 | 10.4 | 13.1 | 15.7 | |
| HR (95% CI) | 0.85 (0.76, 0.96) | | 0.85 (0.65, 1.12) | | 0.76 |
| CV death or first HF | | | | | |
| hospitalization | | | | | |
| Events (%) | 505 (19.3%) | 549 (21.3%) | 94 (23.9%) | 115 (27.1%) | |
| Rate (per 100 py) | 8.4 | 9.4 | 12.9 | 14.6 | |

| | | | |
|---|---|---|---|
| HR (95% CI) | 0.89 (0.79, 1.01) | 0.90 (0.68, 1.19) | 0.86 |

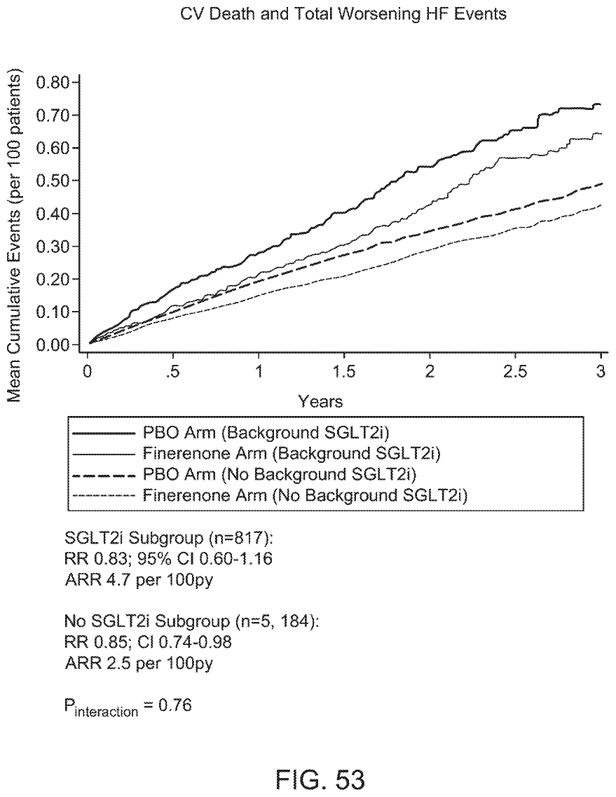

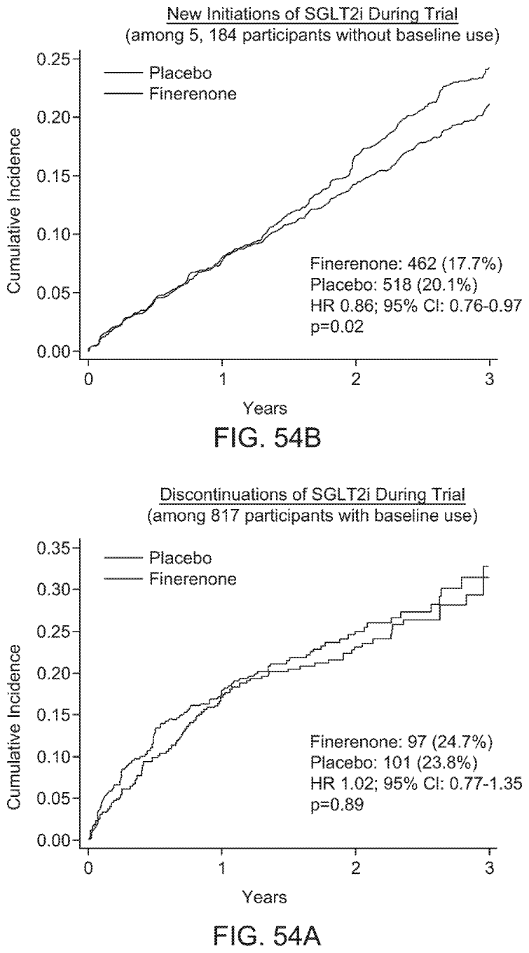

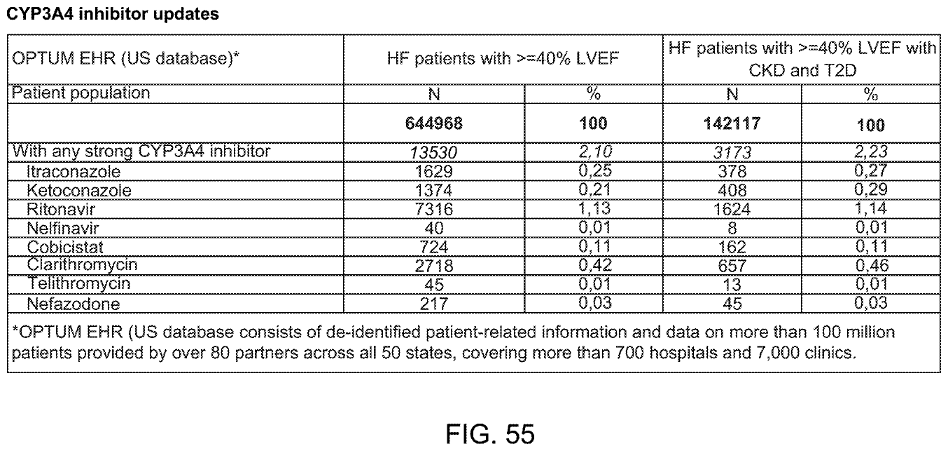

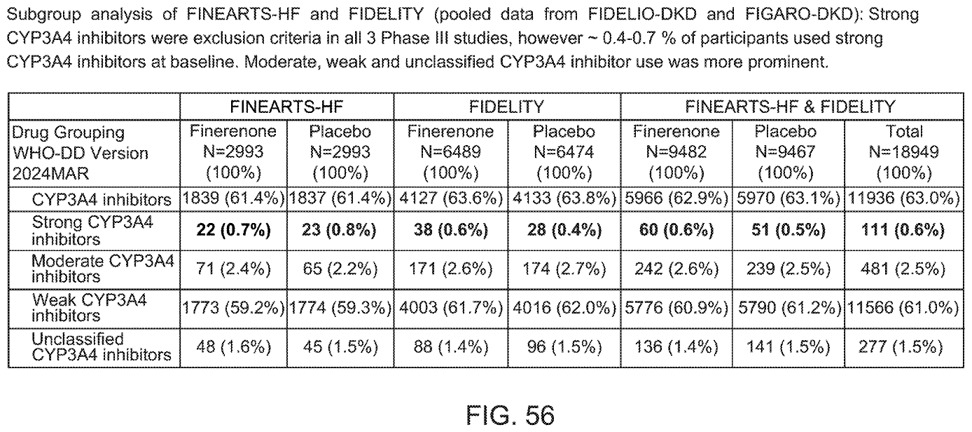

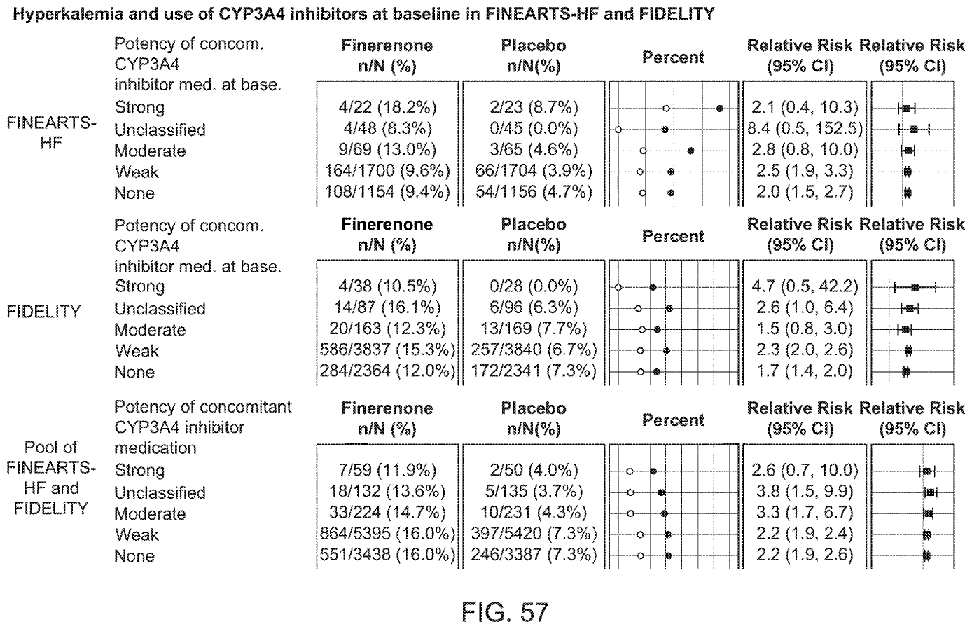

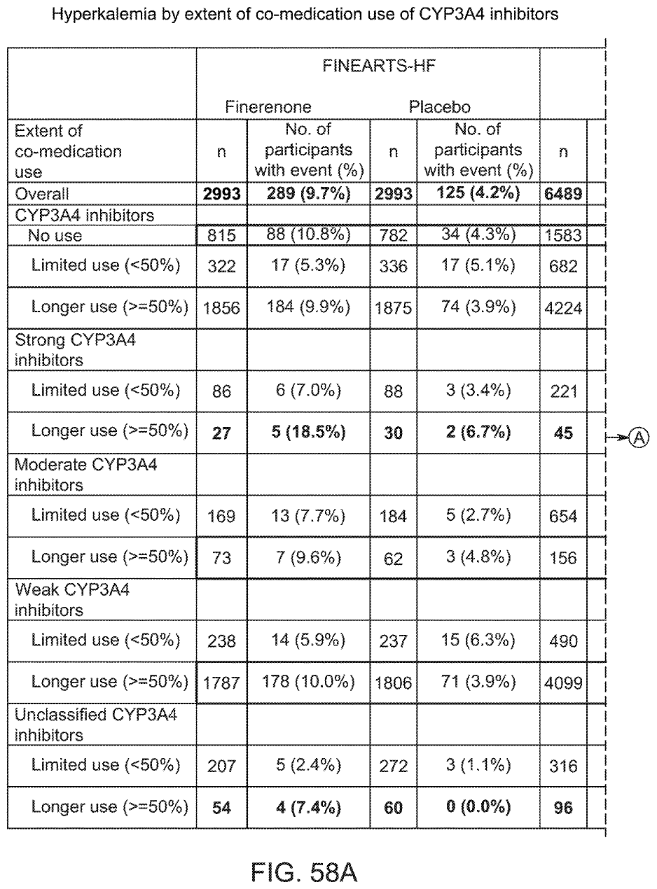

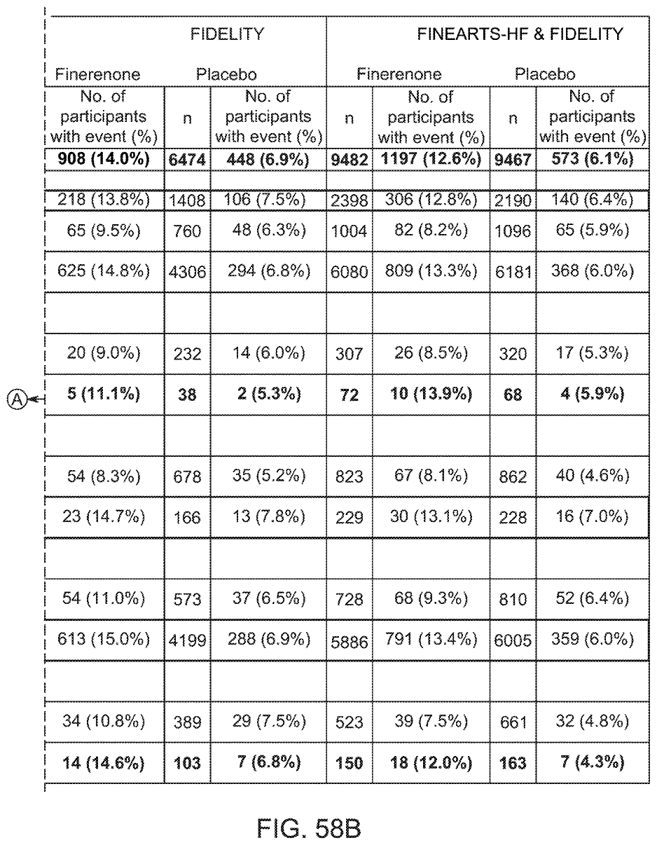

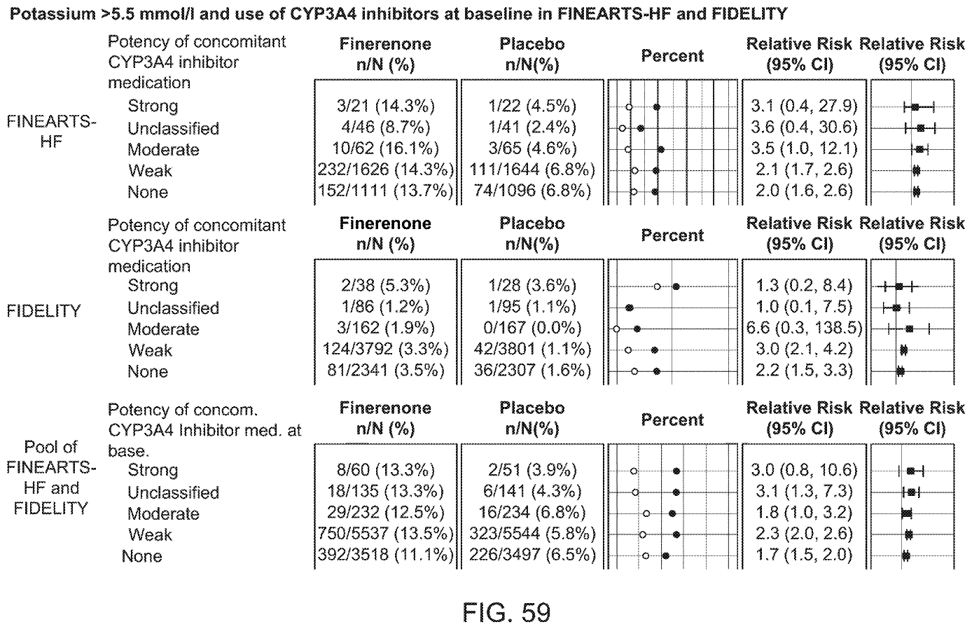

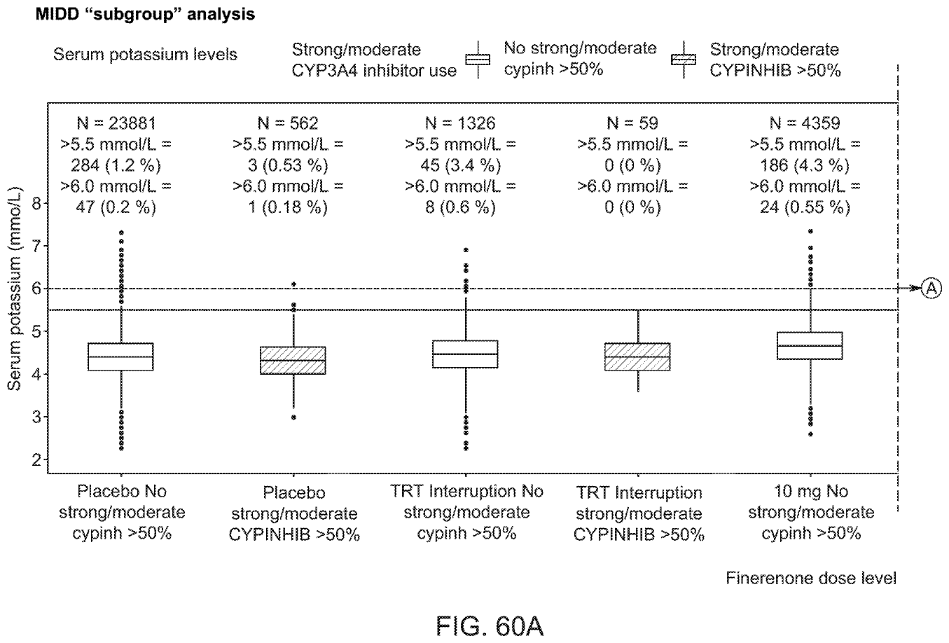

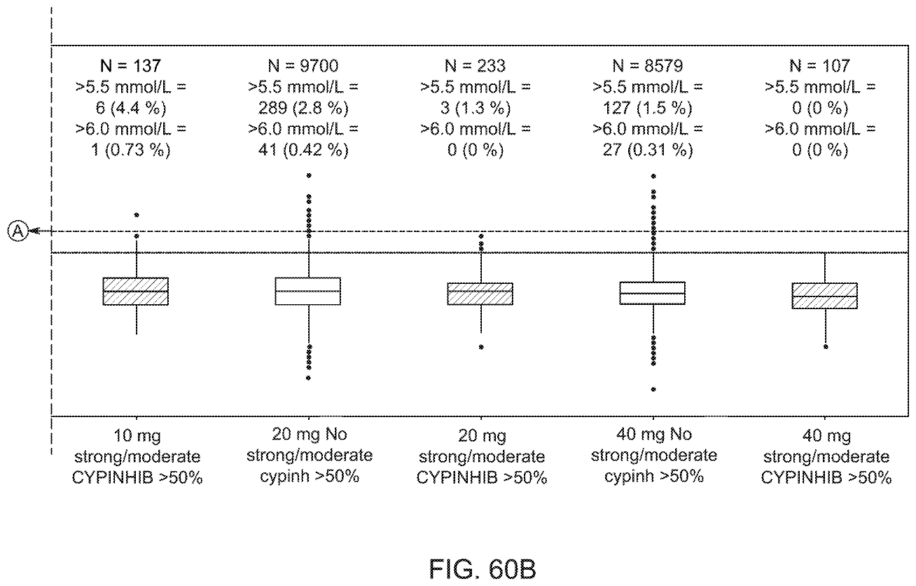

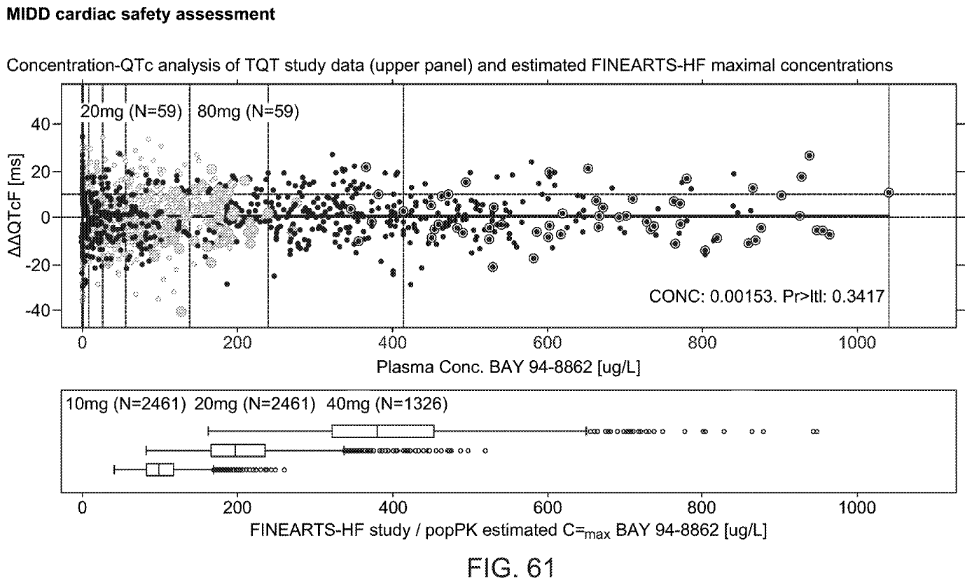

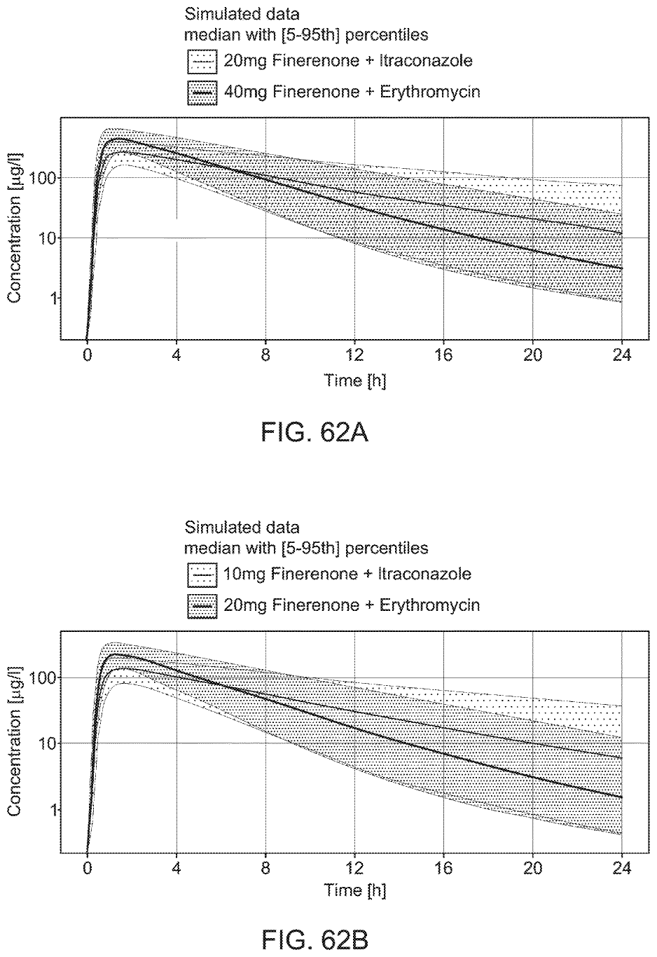

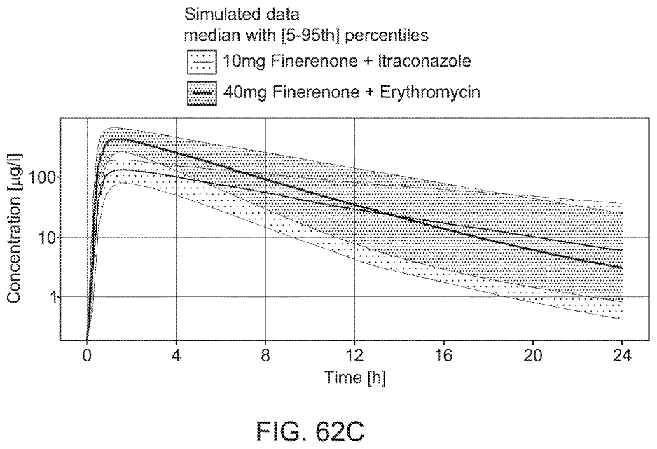

Abbreviations:
CI = confidence interval;
CV = cardiovascular;
HF = heart failure;
HR = hazard ratio;
RR = rate ratio;
SGLT2i = sodium-glucose-co-transporter-2 inhibitor
See FIG. 53 and FIGS. 54A-54B.

TABLE 37
Adverse Events and Other Safety Markers of Interest by Concomitant SGLT2 Inhibitor Use

| | No SGLT2i Use | | SGLT2i Use | |
|---|---|---|---|---|
| | Placebo | Finerenone | Placebo | Finerenone |
| | n = 2,570 | n = 2,601 | n = 423 | n = 392 |
| Any Serious Adverse Events | 1,042 | 995 | 171 | 162 |
| | (40.5%) | (38.3%) | (40.4%) | (41.3%) |
| Any Adverse Events leading to Treatment | 73 | 90 | 10 | 6 |
| Discontinuation | (2.8%) | (3.5%) | (2.4%) | (1.5%) |
| Elevated Serum Creatinine ≥2.5 mg/dl | 78 | 120 | 11 | 21 |
| | (3.1%) | (4.8%) | (2.7%) | (5.5%) |
| Elevated Serum Creatinine ≥3.0 mg/dl | 29 | 50 | 5 | 7 |
| | (1.2%) | (2.0%) | (1.2%) | (1.8%) |
| Elevated Serum Potassium >5.5 mmol/liter | 176 | 358 | 23 | 55 |
| | (7.1%) | (14.2%) | (5.6%) | (14.5%) |
| Elevated Serum Potassium >6.0 mmol/liter | 39 | 76 | 2 | 10 |
| | (1.6%) | (3.0%) | (0.5%) | (2.6%) |
| Reduced Serum Potassium <3.5 mmol/liter | 246 | 118 | 35 | 9 |
| | (9.9%) | (4.7%) | (8.5%) | (2.4%) |
| Investigator Reported Hyperkalemia | 102 | 246 | 23 | 43 |
| | (4.0%) | (9.5%) | (5.4%) | (11.0%) |
| Hyperkalemia leading to Treatment | 3 | 12 | 2 | 1 |
| Discontinuation | (0.1%) | (0.5%) | (0.5%) | (0.3%) |
| Investigator Reported Hyperkalemia leading to | 3 | 14 | 3 | 2 |
| Hospitalization | (0.1%) | (0.5%) | (0.7%) | (0.5%) |
| Systolic Blood Pressure <100 mm Hg | 291 | 435 | 70 | 103 |
| | (11.7%) | (17.2%) | (16.8%) | (27.0%) |

Abbreviations:
SGLT2i = sodium-glucose-co-transporter-2 inhibitor

Treatment-emergent adverse events are defined as any adverse event occurring in any patient who has received at least one dose of study drug and within three days of permanent discontinuation. The data reported on creatinine, potassium, and systolic blood pressure levels were further restricted to patients with at least one assessment.

### Example 28—Influence of Extrinsic Factors on Pharmacokinetic (PK) and Pharmacodynamic (PD) Characteristics of Finerenone

Physiologically based pharmacokinetic (PBPK) modeling and simulation as earlier published and incorporated by reference here (T. Wendl et al., CPT Pharmacometrics Syst. Pharmacol. 2022 February; 11 (2): 199-211. doi: 10.1002/psp4.12746) is used to predict the pharmacokinetic behavior of drugs in humans using preclinical data. It also explores the effects of various physiologic parameters such as age, ethnicity, or disease status on human pharmacokinetics, as well as guide dose and dose regiment selection and aid drug-drug interaction risk assessment.

PBPK model simulations demonstrated that the exposure to finerenone after administering 10 mg of finerenone concomitantly with strong CYP3A4 inhibitors results in comparable exposure to that of 40 mg finerenone without any CYP3A4 inhibitors (with a slightly higher AUC and lower Cmax). This exposure is notably lower than that observed with 40 mg of finerenone taken with moderate CYP3A4 inhibitors.

By conjunction, it can therefore be considered a safe option to down-titrate patients on 40 mg finerenone treatment to 10 mg finerenone (i.e., 4-fold dose reduction), when starting an indicated therapy of concomitant strong CYP3A4 inhibitors. For patients not on 40 mg finerenone treatment, such an option is not available.

PBPK comparative exposure assessment for finerenone and CYP3A4 modulators (CPMX50251, B003537)

In this study, the previously established finerenone PBPK model (T. Wendl et al., CPT Pharmacometrics Syst. Pharmacol. 2022 February; 11 (2): 199-211. doi: 10.1002/psp4.12746) was coupled to independently developed PBPK CYP3A4 perpetrator models in order to predict the extent of interaction considering different doses of finerenone, thereby extending previous results.

### Objectives:

The objective of this analysis was to compare the exposure levels of finerenone at therapeutic doses from 10 up to 40 mg when taken with CYP3A4 inhibitors. A specific aim was to provide evidence for the potential co-administration of strong CYP3A4 inhibitors in certain scenarios, in particular whether a low dose of 10 mg finerenone combined with strong inhibitors leads to lower exposure (based on derived PK parameters AUC and Cmax) than those seen with a high dose of 40 mg finerenone paired with moderate CYP3A4 inhibitors.

Modeling: The previously reported finerenone PBPK model was coupled to established PBPK models of itraconazole, clarithromycin (both strong CYP3A4 inhibitors), erythromycin, verapamil (both moderate CYP3A4 inhibitors), and fluvoxamine (moderate inhibitor, classified as weak inhibitor until 2019), and cimetidine (weak CYP3A4 inhibitor).

The extent of interaction and the PK of finerenone at doses of 10, 20, and 40 mg co-administered with CYP3A4 modulators was predicted through population simulations with a virtual Phase 1 population previously established. For all treatments with CYP3A4 modulators (see Table 38) a control with the same settings, but lacking modulator coadministration was simulated to calculate AUC and Cmax.

For all modulator treatments, the maximum permissible dose was selected in order to reach the maximum inhibitory effect. Duration of treatment was selected using preliminary PBPK simulations to ensure that more than 95% of the maximum effect was reached. Administration of modulators was continued in the DDI model after finerenone administration to maintain the maximum effect. For erythromycin and verapamil, modulator treatments were set according to clinical studies, i.e., study 14504 with erythromycin (PH-37055) and study 16910 with verapamil (PH-38891).

Moderate CYP3A4 inhibitors were allowed in all Phase 3 studies including FINEARTS-HF with a 40 mg finerenone dose option for patients with eGFR >60 mL/min/1.73 m², expanding the tested exposure range.

On the basis of serum potassium and general safety assessments based on FINEARTS-HF data as well as a supplementary Concentration-QTc analysis, moderate CYP3A4 inhibitors concomitantly administered with 40 mg finerenone are considered safe for eligible patients.

PBPK model simulations, qualified with data from several clinical studies, demonstrated that the exposure to finerenone after administering 10 mg of finerenone concomitantly with strong CYP3A4 inhibitors results in comparable exposure to that of 40 mg finerenone without any CYP3A4 inhibitors (with a slightly higher AUC and lower Cmax). However, this exposure is notably lower than that observed with 40 mg of finerenone taken with moderate CYP3A4 inhibitors.

By conjunction, it can therefore be considered a safe option to down-titrate patients on 40 mg finerenone treatment to 10 mg finerenone (i.e., 4-fold dose reduction), when starting an indicated therapy of concomitant strong CYP3A4 inhibitors. For patients not on 40 mg finerenone treatment, such an option is not available. A 2-fold finerenone dose reduction (from 40 to 20 mg or from 20 to 10 mg) when initiating strong CYP3A4 inhibitors can be considered less conservative, however, still results in exposures comparable to those achieved when not dose adapting finerenone under concomitant moderate CYP3A4 inhibitor use and could therefore be considered based on an individual benefit-risk assessment.

TABLE 38
CYP3A4 modulators and simulated treatments that were used
in combination with the established finerenone PBPK model

| | | | Treatment | | |
|---|---|---|---|---|---|
| | | Simulated | schedule of | | Finerenone dose |
| Modulator model | Type | dose | modulator | Offset* | administration |
| Itraconazole | strong | 200 mg | BID over | 1 h | SD on day 7 |
| | inhibitor | | 7 days | | (after last but |
| | | | | | one dose) |
| Clarithromycin | strong | 500 mg | BID over | 2 h | SD on day 8 |
| | inhibitor | | 10 days | | |
| Erythromycin | moderate | 500 mg | TID over | 0 h | SD on day 5 |
| | inhibitor | | 4 days | | |
| | | | (13 times) | | |
| Verapamil | moderate | 120/240 mg | 120 mg | 6 h | SD on day 4 |
| | inhibitor | | on day 1, | | |
| | | | 240 mg | | |
| | | | on day 2, | | |
| | | | 3 and 4 | | |
| Fluvoxamine | weak | 100 mg | BID over | 1 h | SD on day 8 |
| | inhibitor | | 10 days | | |
| Cimetidine | weak | 800 mg | BID over | 0 h | SD on day 3 |
| | inhibitor | | 96 h | | |

Administration of perpetrators was continued in the DDI model after finerenone administration to maintain the maximum effect, for erythromycin and verapamil, perpetrator treatments were set according to clinical studies, i.e., 14504 (with erythromycin, PH-37055), 16910 (with verapamil, PH-38891).
*Time interval between perpetrator administration and finerenone dose
Day 1 represents 0 to 24 h, day 2 from 24 to 48 h, etc.
Abbreviations: BID = twice daily; TID = three times a day; SD = single dose.

Results: Population exposures (AUC, Cmax) of finerenone after single doses of 10, 20, and 40 mg without perpetrator and with concomitant weak, moderate and strong CYP3A4 inhibitors at their highest permissible doses were simulated.

For comparisons between finerenone doses and CYP3A4 inhibitors, 40 mg finerenone concomitant with 500 mg TID erythromycin or 240 mg OD verapamil were used as reference scenarios, as these combinations were considered to result in the highest representative finerenone exposures that were allowed in the FINEARTS-HF trial.

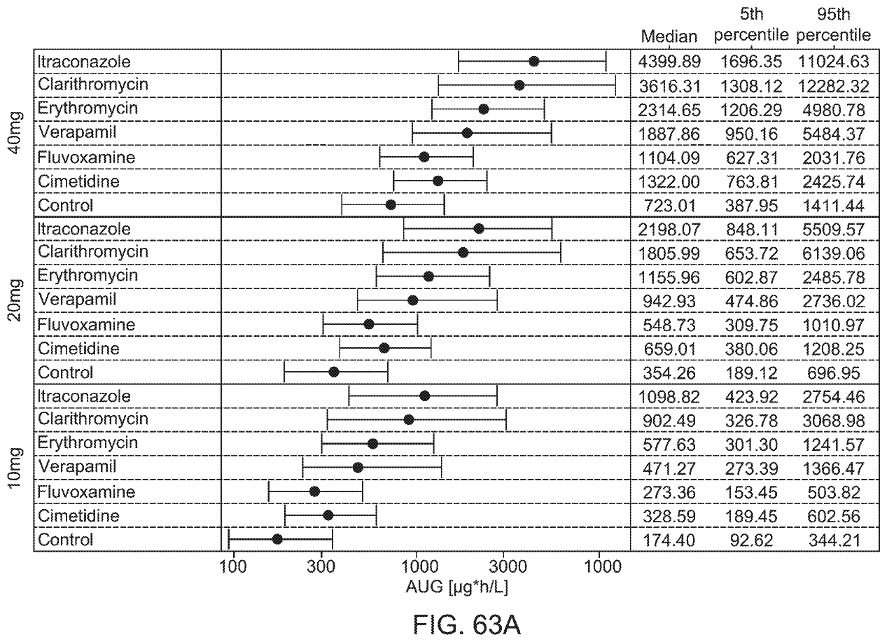

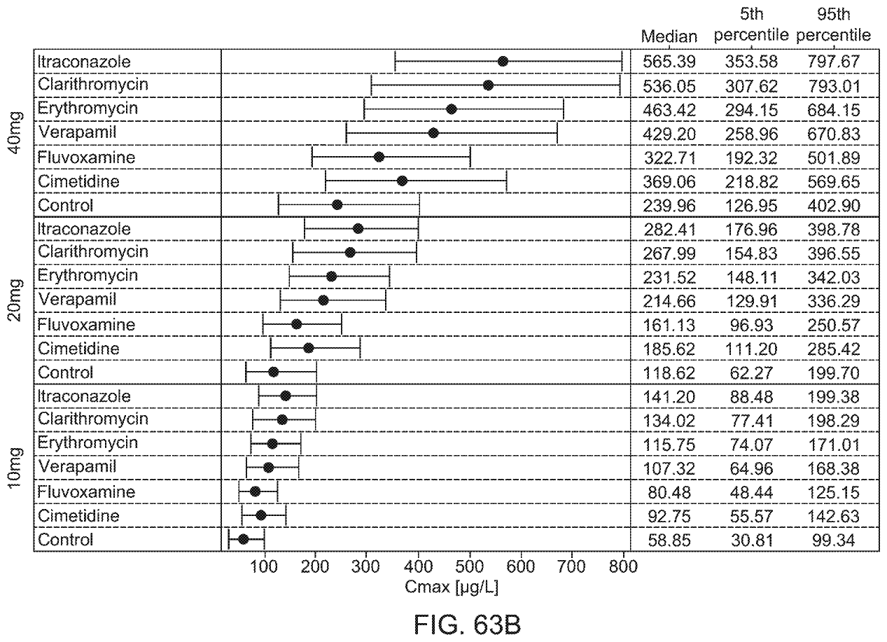

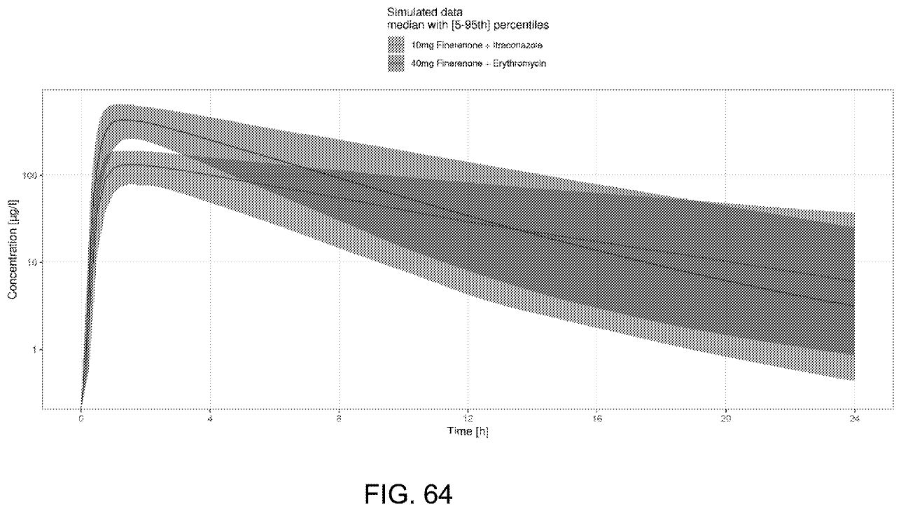

A single 10 mg dose of finerenone concomitant with strong CYP3A4 inhibitors (200 mg BID itraconazole or 500 mg BID clarithromycin) resulted in lower exposures (AUC, Cmax) than the reference scenario at the median as well as at the 5th and 95th percentiles. For the scenarios with 20 mg finerenone concomitant with strong inhibitors, the Cmax values are also below the reference scenario at corresponding percentiles. The AUC values of 20 mg finerenone concomitant with itraconazole remain below the AUC of the reference scenario at the 5th percentile and are comparable in the medians, however, the 95th percentiles exceed the 95th percentiles of the reference scenarios. See FIG. 63A.

FIG. 63A and FIG. 63B: Forest plot of simulated absolute AUC (top) and Cmax (bottom) values of finerenone in doses of 10, 20 and 40 mg under concomitant administration of strong (itraconazole and clarithromycin), moderate (erythromycin and verapamil) and weak (cimetidine and fluvoxamine) CYP3A4 inhibitors. Dots represent medians, whiskers extend from 5th to 95th percentiles.

Even with concomitant moderate inhibitors like erythromycin or strong inhibitors like itraconazole, the terminal half-life of finerenone remains relatively short, i.e., within 4 to 5 hours with erythromycin and 6 to 7 hours in the median with itraconazole co-administration compared to 2 to 3 hours in the case of finerenone alone. Consequently, a new steady state after multiple dosing or down-titration of the finerenone dose from 40 to 10 mg is reached within a short period of time and no significant delays are expected.

Conclusions: The PBPK model simulations demonstrated that the exposure to finerenone after administering 10 mg of finerenone concomitantly with strong CYP3A4 inhibitors results in comparable exposure to that of 40 mg of finerenone without any CYP3A4 inhibitors (with a slightly higher AUC and lower Cmax). This exposure is notably lower than that observed with 40 mg of finerenone taken concomitantly with moderate CYP3A4 inhibitors. See FIGS. 63A-63B.

### Conclusions:

PBPK simulations show that patients on 40 mg finerenone could be down-titrated to 10 mg finerenone (i.e., 4-fold dose reduction) when concomitantly starting the use of strong CYP3A4 inhibitors resulting in a finerenone exposure that can be considered safe. For patients not eligible for treatment with 40 mg finerenone or not currently treated on that dose level, such an option is not available.

### Example 29a—Concomitant Use of Strong CYP3A4 Inhibitors During Finerenone Therapy

Contraindication 1: Finerenone is contraindicated in patients taking concomitant medications that are strong CYP3A4 inhibitors (e.g., itraconazole, ketoconazole, ritonavir, nelfinavir, cobicistat, clarithromycin, telithromycin and nefazodone).

Contraindication 2: Finerenone is contraindicated in patients taking concomitant medications that are strong CYP3A4 inhibitors (e.g., itraconazole, ketoconazole, ritonavir, nelfinavir, cobicistat, clarithromycin, telithromycin and nefazodone) without dose adapting finerenone appropriately.

### Dosing:

Aa) Patients on 10 mg or 20 mg Finerenone cannot initiate concomitant strong CYP3A4 inhibitor treatment. Heart Failure patients or patients who are suffering from heart failure and are stable on 40 mg Finerenone should continue treatment only if the dose is reduced to 10 mg (4-fold dose reduction, 2 dose steps) during concomitant treatment with strong CYP3A4 inhibitors. Monitor serum potassium during drug initiation or dosage adjustment of either Finerenone (Kerendia) or the strong CYP3A4 inhibitor
Ab) Concomitant use of Finerenone target dose of 10 and 20 mg with strong CYP3A4 inhibitors is contraindicated. Patients on target dose of 40 mg finerenone must be down-titrated to 10 mg when using with strong CYP3A4 inhibitors.

### OR:

Finerenone is a CYP3A4 substrate. Concomitant use with a strong CYP3A4 inhibitor increases finerenone exposure, which may increase the risk of finerenone-induced adverse reactions. Concomitant use of finerenone in patients taking a target dose of 10 and 20 mg with strong CYP3A4 inhibitors is contraindicated. Patients taking a target dose of 40 mg finerenone must be down-titrated to 10 mg when using with strong CYP3A4 inhibitors.

Special warnings: Patients who suffering from heart failure and stable on 40 mg finerenone who require concomitant administration of a strong CYP3A4 inhibitor must down-titrate finerenone to 10 mg (4-fold, 2 dose steps) for the time of concomitant use. Consider additional serum potassium monitoring according to patient characteristics and withdraw finerenone treatment as needed. (For patients on 10 mg or 20 mg without concomitant CYP3A4 inhibitors, an appropriate dose reduction is not possible, and the concomitant use is contraindicated. Consider alternative drugs or withdraw finerenone treatment.)

Interaction: Strong CYP3A4 inhibitor is contraindicated unless the finerenone dose is reduced adequately. Simulations suggest that patients stable on 40 mg finerenone can continue treatment if the dose is reduced to 10 mg (4-fold, 2 dose steps) for the time of concomitant strong CYP3A4 inhibitors use. Serum potassium may increase, and therefore, monitoring of serum potassium is recommended. For patients on 10 mg or 20 mg without concomitant CYP3A4 inhibitors, an appropriate dose reduction is not possible. For patients taking strong CYP3A4 inhibitors, no safe initiation of finerenone treatment is possible.

### Example 29b—Concomitant Use of Strong CYP3A4 Inhibitors During Finerenone Therapy

In a less conservative scenario, it can still be considered a safe option to down-titration patients one dose step, i.e., from 40 mg to 20 mg or from 20 mg to 10 mg, when starting an indicated therapy of concomitant strong CYP3A4 inhibitors. For patients on 10 mg without concomitant CYP3A4 inhibitors, an appropriate dose reduction is not possible.

### Example 30—Concomitant Use of Moderate or Weak CYP3A4 Inhibitors During Finerenone Therapy

Finerenone is a CYP3A4 substrate. Concomitant use with a moderate or weak CYP3A4 inhibitor increases finerenone exposure, which may increase the risk of finerenone adverse reactions. Monitor serum potassium during drug initiation or dosage adjustment of either finerenone or the moderate or weak CYP3A4 inhibitor and adjust finerenone dosage as appropriate. Patients on 40 mg finerenone should be down-titrated to 20 mg in case of concomitant use with moderate CYP3A4 inhibitors.

### OR:

Finerenone is a CYP3A4 substrate. Concomitant use with a moderate or weak CYP3A4 inhibitor increases finerenone exposure, which may increase the risk of finerenone-induced adverse reactions. Monitor serum potassium during drug initiation or dosage adjustment of either finerenone or the moderate or weak CYP3A4 inhibitor and adjust finerenone dosage as appropriate. Patients taking 40 mg of finerenone should reduce their dose to 20 mg if used concurrently with moderate CYP3A4 inhibitors.

### OR:

Finerenone is a CYP3A4 substrate. Concomitant use with a moderate or weak CYP3A4 inhibitor increases finerenone exposure, which may increase the risk of finerenone adverse reactions. Monitor serum potassium during drug initiation or dosage adjustment of either finerenone or the moderate or weak CYP3A4 inhibitor and adjust finerenone dosage as appropriate. In cases where patients are on 40 mg finerenone, the dose should be adjusted to 20 mg if they are using moderate CYP3A4 inhibitors at the same time.

### Example 31—Population PBPK Simulated Concentration-Time Profiles and Derived PK-Parameters of Population Simulations

In the present study, a finerenone physiologically based pharmacokinetic (PBPK) model for adults was applied with finerenone doses of 2.5, 5, 10, 20 and 40 mg in combination with several other PBPK CYP3A4 perpetrator models, developed and verified independently of finerenone. The study aims to predict the impact of strong CYP3A4 inhibitors in certain dosing scenarios in comparison to the predicted effect of moderate CYP3A4 inhibitors on 10, 20 and 40 mg finerenone.

The objective of this analysis was to provide a comparative exposure assessment for finerenone at different doses co-administered with CYP3A4 perpetrators with the aim to create evidence for a possible concomitant medication of strong inhibitors of CYP3A4 in certain scenarios. For this purpose, population exposure (PK, AUC, Cmax) was simulated for finerenone at doses of 2.5, 5, 10, 20, and 40 mg without and with concomitant administration of weak (cimetidine and fluvoxamine), moderate (erythromycin and verapamil) and strong (itraconazole and clarithromycin) inhibitors of CYP3A4.

It was of particular interest to explore whether a lower dose of finerenone, when combined with a strong inhibitor, results in derived parameters such as the AUC and Cmax remaining below the corresponding values observed with a higher dose of finerenone without or alongside a moderate inhibitor.

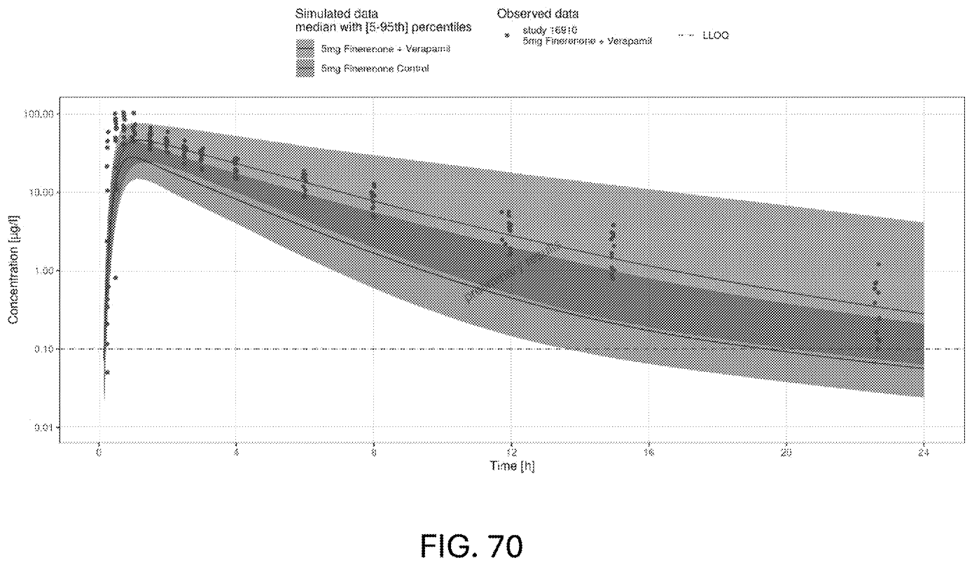

Erythromycin and verapamil are both moderate CYP3A4 inhibitors, but erythromycin has a slightly stronger inhibitory effect on finerenone such that 40 mg finerenone concomitant with 500 mg TID erythromycin is considered to generate the highest finerenone exposure. Hence, this scenario served as a reference scenario in FIG. 70 and FIG. 64.

FIG. 70 compares 10 mg finerenone concomitant with 200 mg BID itraconazole to this reference and FIG. 64 compares 20 mg finerenone concomitant with 200 mg BID itraconazole to it. Itraconazole is a strong CYP3A4 inhibitor.

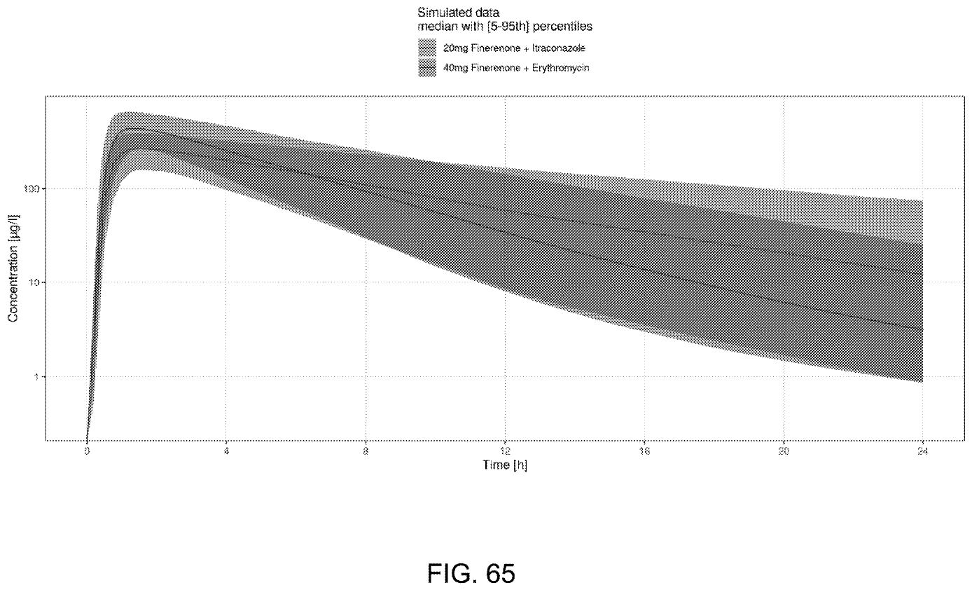

FIG. 70 shows that the concentrations of 10 mg finerenone concomitant with 200 mg BID itraconazole are below the concentrations of 40 mg finerenone concomitant with 500 mg TID erythromycin for more than 12 h after finerenone administration. FIG. 65 shows that the concentrations of 20 mg finerenone concomitant with 200 mg BID itraconazole are below the concentrations of 40 mg finerenone concomitant with 500 mg TID erythromycin for more than 6 h after finerenone administration.

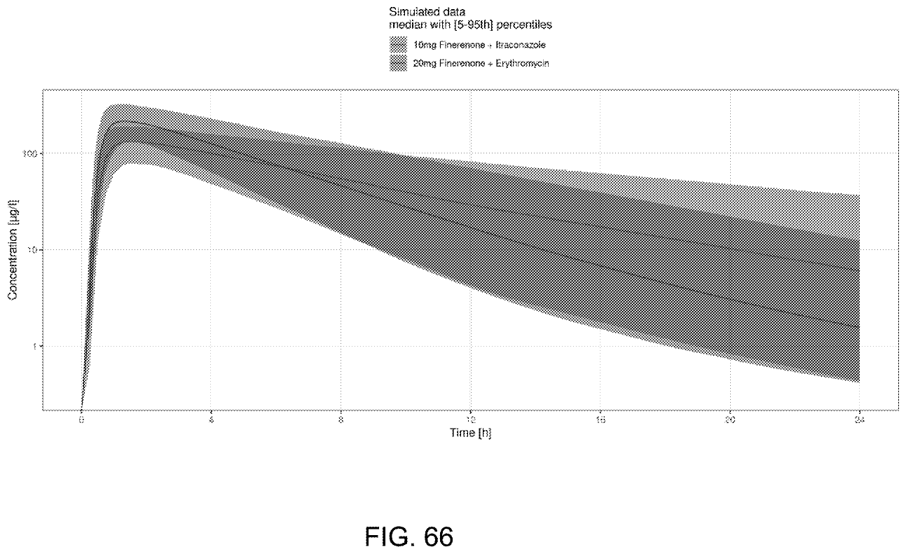

To evaluate whether subjects on 20 mg OD finerenone can be down-titrated to 10 mg when receiving concomitant treatment with a strong CYP3A4 inhibitor, FIG. 66 compares 10 mg finerenone concomitant to 200 mg BID itraconazole to 20 mg finerenone concomitant with 500 mg TID erythromycin. The results demonstrate that the concentrations of 10 mg finerenone concomitant with 200 mg BID itraconazole are below the concentrations of 20 mg finerenone concomitant with 500 mg TID erythromycin for more than 6 h after finerenone administration.

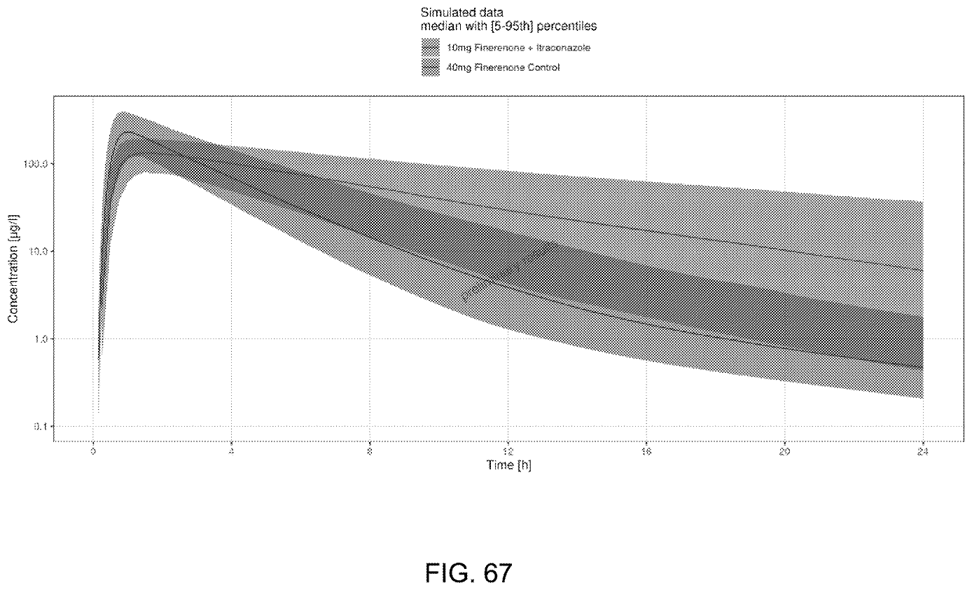

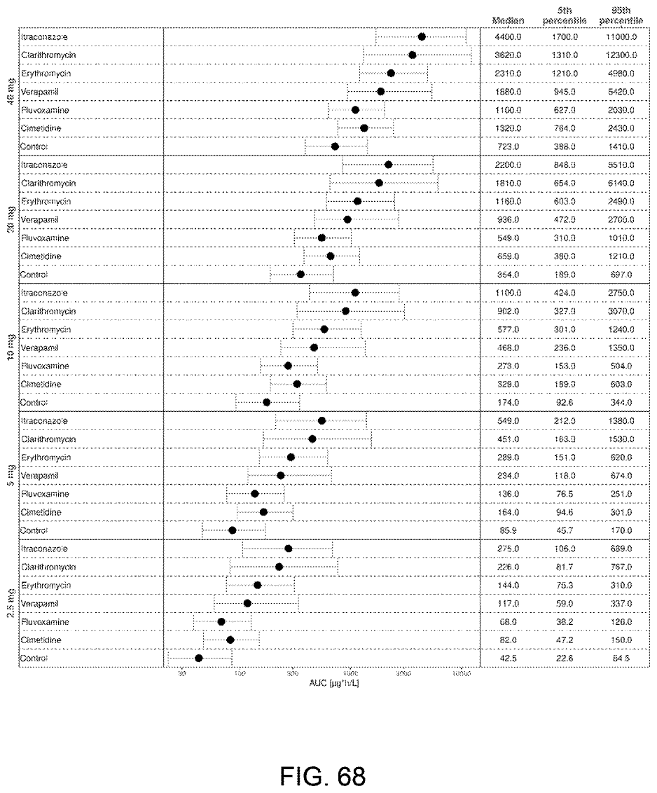

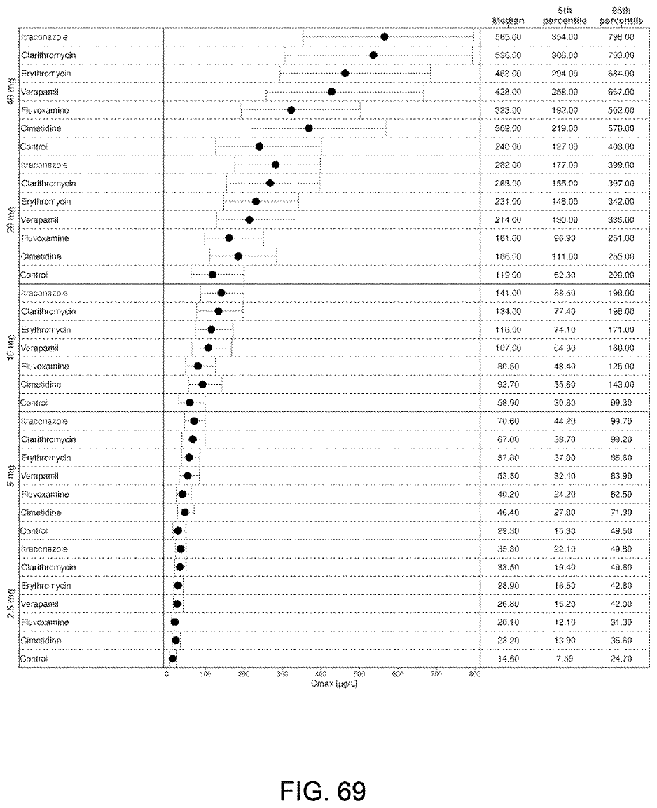

FIG. 67 shows a comparison of 10 mg finerenone concomitant with 200 mg BID itraconazole and 40 mg finerenone alone demonstrating that Cmax is lower in the first scenario, while finerenone terminal half-life is extended from 2-3 hours (without CYP3A4 inhibition) to 6-7 hours (with strong CYP3A4 inhibition).

FIGS. 64-67 also demonstrate that the terminal half-lives of finerenone in different doses remain relatively short under inhibitor co-administration. Finerenone under erythromycin co-administration shows a terminal half-life within 4 to 5 hours in the median and 6 to 7 hours in the median with itraconazole.

FIGS. 66-67 compare various scenarios for finerenone, examining different doses of finerenone in combination with varying strengths of CYP3A4 inhibitors. The figures depict median and 5^{th} to 95^{th} percentiles of AUC (FIG. 66) and Cmax (FIG. 67).

FIG. 66 shows that if the median AUC for a combination with a certain inhibitor is higher than with another inhibitor of the same category, the 5^{th} or 95^{th} percentiles are not necessarily higher. For example, the median AUC for 40 mg finerenone under erythromycin treatment is higher than under verapamil treatment, but the 95^{th} percentile under erythromycin treatment is lower than under verapamil treatment. The same applies to strong inhibitors, where the median finerenone AUC under clarithromycin co-administration is lower than under itraconazole co-administration, but the AUC in the 95^{th} percentile is still higher. Therefore, only inhibitor categories are compared below and the spectrum of the AUC distribution in an inhibitor category can be made up of the percentiles of different inhibitors.

As moderate, but not strong, inhibitors were allowed as comedication in the clinical trial, the combination of 40 mg finerenone and moderate inhibitors was considered as the combination with the highest clinically tested exposure. This combination is called reference scenario in the following.

The AUC following the administration of 10 mg finerenone in combination with strong inhibitors is clearly lower than that of the reference scenario and has an overlapping exposure compared to 40 mg of finerenone alone without any CYP3A4 inhibitor co-administration (slightly higher in AUC, lower in Cₘₐₓ). For the combination of 20 mg finerenone with strong inhibitors, only the median and the 5^{th} percentile are below the corresponding reference percentiles, but not the 95^{th} of the AUC values. Therefore, a small subset of the population might experience higher exposures in the latter combinations compared to the former. The same applies to 10 mg finerenone concomitant with strong inhibitors vs. 20 mg finerenone concomitant with moderate inhibitors, where only the median and the 5^{th} percentile are below the AUC of the corresponding reference percentiles, but not the 95^{th} percentile. In terms of Cₘₐₓ, the PBPK predicted population Cₘₐₓ values after 10 mg of finerenone concomitant with strong inhibitors largely overlap with the 20 mg finerenone control and partially overlap with the 40 mg finerenone control.

Finerenone is almost dose-proportional in the investigated dose range, so the statement above also applies to lower dosing, e.g. a 5 mg finerenone dose concomitant with strong CYP3A4 inhibitors resulted in lower exposure than 20 mg with moderate inhibitors and 2.5 mg with strong CYP3A4 inhibitors resulted in lower exposure than 10 mg with moderate inhibitors.

Furthermore, AUCR and CₘₐₓR between selected scenarios were calculated and are shown in Tables D-G. Table D shows AUCR and CₘₐₓR for 10 mg finerenone concomitant with 200 mg BID itraconazole vs. 40 mg finerenone control, Table E shows AUCR and CₘₐₓR for 10 mg finerenone concomitant with 200 mg BID itraconazole vs. 40 mg finerenone concomitant with 500 mg TID erythromycin, Table F shows AUCR and CₘₐₓR for 10 mg finerenone concomitant with 500 mg BID clarithromycin vs. 40 mg finerenone control and Table G shows AUCR and CₘₐₓR for 10 mg finerenone concomitant with 500 mg BID clarithromycin vs. 40 mg finerenone concomitant with 500 mg TID erythromycin.

As finerenone is almost dose linear in the investigated dose range, the statement above also applies to lower dosing, meaning that halving or quartering the doses leads to similar exposure proportions.

TABLE D
AUCR and CₘₐₓR of 10 mg finerenone concomitant with 200 mg
BID itraconazole vs. 40 mg finerenone control
10 mg finerenone + itraconazole vs. 40 mg finerenone control

| | P5 | P25 | P50 | P75 | P95 | arithMean | arithSD | geoMean | geoSD | geoCV |
|---|---|---|---|---|---|---|---|---|---|---|
| AUCR | 0.82 | 1.19 | 1.53 | 1.98 | 2.74 | 1.63 | 0.63 | 1.52 | 1.45 | 0.39 |
| CₘₐₓR | 0.44 | 0.50 | 0.58 | 0.66 | 0.80 | 0.59 | 0.12 | 0.58 | 1.21 | 0.19 |

Abbreviations: P5, P25, P50, P75, P95 = 5^{th}, 25^{th}, 50^{th}, 75^{th}, 95^{th} percentiles, arithMean = arithmetic mean, arithSD = arithmetic standard deviation, geoMean = geometric mean, geoSD = geometric standard deviation, geoCV = geometric coefficient of variation

TABLE E
AUCR and CₘₐₓR of 10 mg finerenone concomitant with 200 mg
BID itraconazole vs. 40 mg finerenone concomitant with 500 mg TID erythromycin
10 mg finerenone + itraconazole vs. 40 mg finerenone + erythromycin

| | P5 | P25 | P50 | P75 | P95 | arithMean | arithSD | geoMean | geoSD | geoCV |
|---|---|---|---|---|---|---|---|---|---|---|
| AUCR | 0.28 | 0.38 | 0.47 | 0.57 | 0.77 | 0.49 | 0.15 | 0.47 | 1.35 | 0.31 |
| CₘₐₓR | 0.26 | 0.28 | 0.30 | 0.32 | 0.35 | 0.30 | 0.03 | 0.30 | 1.09 | 0.09 |

Abbreviations: P5, P25, P50, P75, P95 = 5^{th}, 25^{th}, 50^{th}, 75^{th}, 95^{th} percentiles, arithMean = arithmetic mean, arithSD = arithmetic standard deviation, geoMean = geometric mean, geoSD = geometric standard deviation, geoCV = geometric coefficient of variation

TABLE F
AUCR and CₘₐₓR of 10 mg finerenone concomitant with 500 mg BID
clarithromycin vs. 40 mg finerenone control
10 mg finerenone + clarithromycin vs. 40 mg finerenone control

| | P5 | P25 | P50 | P75 | P95 | arithMean | arithSD | geoMean | geoSD | geoCV |
|---|---|---|---|---|---|---|---|---|---|---|
| AUCR | 0.70 | 0.98 | 1.22 | 1.63 | 2.52 | 1.37 | 0.57 | 1.27 | 1.47 | 0.40 |
| CₘₐₓR | 0.44 | 0.49 | 0.55 | 0.61 | 0.73 | 0.56 | 0.09 | 0.55 | 1.17 | 0.16 |

Abbreviations: P5, P25, P50, P75, P95 = 5^{th}, 25^{th}, 50^{th}, 75^{th}, 95^{th} percentiles, arithMean = arithmetic mean, arithSD = arithmetic standard deviation, geoMean = geometric mean, geoSD = geometric standard deviation, geoCV = geometric coefficient of variation

TABLE G
AUCR and CₘₐₓR of 10 mg finerenone concomitant with 500 mg
BID clarithromycin vs. 40 mg finerenone concomitant with 500 mg TID erythromycin
10 mg finerenone + clarithromycin vs. 40 mg finerenone + erythromycin

| | P5 | P25 | P50 | P75 | P95 | arithMean | arithSD | geoMean | geoSD | geoCV |
|---|---|---|---|---|---|---|---|---|---|---|
| AUCR | 0.25 | 0.31 | 0.38 | 0.48 | 0.66 | 0.41 | 0.13 | 0.39 | 1.34 | 0.30 |
| CₘₐₓR | 0.25 | 0.27 | 0.29 | 0.30 | 0.32 | 0.29 | 0.02 | 0.28 | 1.07 | 0.07 |

Abbreviations: P5, P25, P50, P75, P95 = 5^{th}, 25^{th}, 50^{th}, 75^{th}, 95^{th} percentiles, arithMean = arithmetic mean, arithSD = arithmetic standard deviation, geoMean = geometric mean, geoSD = geometric standard deviation, geoCV = geometric coefficient of variation

Conclusions: Population exposures were simulated for finerenone at doses of 2.5, 5, 10, 20, and 40 mg without perpetrator administration and with concomitant weak (cimetidine), weak to moderate (fluvoxamine), moderate (erythromycin and verapamil) and strong (itraconazole and clarithromycin) CYP3A4 inhibitors. In all tested scenarios, finerenone was given as a single dose as in Phase 1 DDI studies. Even with concomitant moderate inhibitors like erythromycin or strong inhibitors like itraconazole, the terminal half-life of finerenone remains relatively short, i.e. within 4 to 5 hours with erythromycin and 6 to 7 hours in the median with itraconazole co-administration compared to 2 to 3 hours in the case of finerenone alone. Consequently, a new steady state after multiple dosing or down-titration of the finerenone dose from 40 to 10 mg is expected to be reached within a short period of time and no significant delays are expected.

Erythromycin and itraconazole can be considered as strong representatives within their categories of moderate (erythromycin) and strong (itraconazole) inhibitors, and hence, are suited to compare maximal exposures between these categories. However, the upper end (95^{th} percentile) of the AUC after concomitant administration of clarithromycin exceeds the 95^{th} percentile of the AUC after concomitant administration of itraconazole. The same applies to verapamil and erythromycin co-administration. Taken together, the population simulations clearly demonstrate that the AUC and Cmax following the administration of 10 mg finerenone in combination with itraconazole or clarithromycin are lower than those of the reference scenario involving 40 mg finerenone concomitant with erythromycin or verapamil.

In terms of Cmax, the predicted population Cmax after 10 mg of finerenone concomitant with itraconazole or clarithromycin largely overlap with the 20 mg finerenone control and still partially overlap with the 40 mg finerenone control.

A single dose administration of 10 mg finerenone concomitant with itraconazole increases geometric mean (geoMean) finerenone AUC (geoMean AUCR=1.52) and decreases geoMean finerenone Cₘₐₓ (geoMean CₘₐₓR=0.58) compared to 40 mg finerenone alone. The same applies to finerenone concomitant with clarithromycin, which achieves a geoMean AUCR of 1.27 and a geoMean CₘₐₓR of 0.55.

If 10 mg finerenone concomitant with a strong inhibitor is compared to 40 mg concomitant with erythromycin, finerenone geoMean AUCR and CₘₐₓR is 0.47 and 0.30, respectively, for itraconazole and 0.39 and 0.28, respectively for clarithromycin.

As finerenone is almost dose linear in the investigated dose range, the statements above also apply to lower dosing, meaning that halving or quartering the doses leads to similar exposure proportions.

In the present study as in the previous finerenone PBPK DDI study, there is some uncertainty regarding the predicted strength of finerenone interaction with the named perpetrators. The geometric mean fold error (GMFE) on AUCR and CₘₐₓR derived from the simulated combinations of the established DDI network on OSP can serve as a measure of mean uncertainty. For all simulated combinations of the DDI network on OSP, it was calculated to be approximately 1.38 on AUC and 1.33 on Cₘₐₓ. As a result of that, deviations in this range for all predictions may be expected. This can be broken down to the different types of mechanisms. For all included competitive inhibition simulations, i.e. the combinations with itraconazole, fluvoxamine or cimetidine, the GMFE on AUCR was calculated to be 1.39, and 1.25 on CₘₐₓR. For all included MBI simulations, i.e. all combinations with clarithromycin, erythromycin or verapamil, the GMFE on AUCR was calculated to be 1.26, and 1.22 on CₘₐₓR.

In summary, the analysis provides a comprehensive comparative exposure assessment for finerenone at different doses without and with concomitant CYP3A4 inhibitors of various strength. Overall, due to finerenone's dose linearity, it can be stated that exposure levels of finerenone at certain dose concomitant with moderate CYP3A4 inhibitors are always higher than that of finerenone at a quarter of the original dose concomitant with a strong inhibitor. This supports the idea that patients on finerenone doses of 10, 20 and 40 mg can be down-titrated to 2.5, 5 and 10 mg, respectively, if the additional use of a strong CYP3A4 inhibitor is indicated.

### NUMBERED EMBODIMENTS OF THE DISCLOSURE

Notwithstanding the appended claims, the disclosure sets forth the following numbered embodiments:
1. A method of preventing or treating heart failure in a patient, comprising administering to the patient a therapeutically effective amount of a compound of Formula (I)

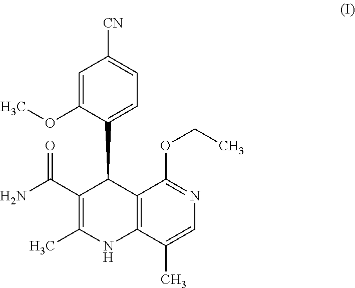 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof.
2. The method according to embodiment 1, wherein the heart failure is selected from the group consisting of symptomatic heart failure, heart failure with improved ejection fraction (HFimpEF), heart failure with mid-range ejection fraction (HFmrEF), heart failure preserved ejection fraction (HFpEF), heart failure with reduced ejection fraction (HFrEF), chronic heart failure (CHF), congestive heart failure, acute heart failure, chronic heart failure, worsening chronic heart failure (WCHF), and hospitalization for heart failure.
3. The method according to any one of embodiments 1 to 2, wherein the patient has a left ventricular ejection fraction (LVEF) of equal to or above 30%, 35%, 39%, 40%, 45%, 49%, 50%, 55%, 60%, 65% or 70%.
4. The method according to any one of embodiments 1 to 3, wherein the patient has an estimated glomerular filtration rate (eGFR) selected from the group consisting of equal to or above 15, 20, 25, 30, 35, 40, 41, 45, 50, 55, 60, 65, 70, 75, 80, 85, and 90 mL/min/1.73 m².
5. The method according to any one of embodiments 1 to 4, wherein the patient has a Urine Albumin to Creatinine Ratio (UACR) of equal to or above 5 to equal to or below 5000 mg/g.
6. The method according to any one of embodiments 1 to 5, wherein the patient has an eGFR of equal to or below 15 mL/min/1.73 m² and a UACR of equal to or above 300 mg/g.
7. The method according to any one of embodiments 1 to 6, wherein the patient has a potassium level of equal to or above 3, 3.5, 4, 4.5, 4.8, 5, 5.5, 6, 6.5, 7.0 or 7.5 mmol/L.
8. The method according to any one of embodiments 1 to 7, wherein the patient has a sodium level selected from the group consisting of equal to or above 100, 110, 115, 117, 120, 125, 130, 135, 138, 140, 141, 144, 145, 150, 154 and 155 mmol/L.
9. The method according to any one of embodiments 1 to 8, wherein the patient has an urea level of equal to or above 4 mg/dL.
10. The method according to any one of embodiments 1 to 9, wherein the patient has a N-terminal prohormone of brain natriuretic peptide (NT-proBNP) of equal to or above 300 μg/ml.
11. The method according to any one of embodiments 1 to 10, wherein the patient further receives a medicament selected from the group consisting of loop diuretics, beta-blockers, ACE inhibitors (ACEi), angiotensin-receptor-blocker (ARB), Angiotensin Receptor-Neprilysin Inhibitor (ARNI), calcium channel blockers, Sodium-glucose Cotransporter-2 Inhibitor (SGLT-2i), glucagon-like peptide-1 (GLP-1) agonists, CYP3A4 inducers, CYP3A4 inhibitors and combinations thereof.
12. The method according to any one of embodiments 1 to 11, wherein the patient further receives a Sodium-glucose Cotransporter-2 Inhibitor (SGLT-2i) and a glucagon-like peptide-1 (GLP-1) agonist.
13. The method according to any one of embodiments 1 to 12, wherein the patient further receives any one of the medicaments or comedications selected from the group consisting of CYP3A4 inducers, CYP3A4 inhibitors and combinations thereof.
14. The method according to any one of embodiments 1 to 13, wherein the amount of compound (I) is administered as a dose of 10, 20, 40 and 60 mg per day.
15. The method according to any one of embodiments 1 to 14, wherein the amount of compound (I) is administered as a dose of 10, 20 and 40 mg per day.
16. The method according to any one of embodiments 1 to 15, wherein the amount of compound (I) is administered as a dose of 10, 20 and 40 mg per day.
17. The method according to any one of embodiments 1 to 16, wherein the daily dose the patient receives is adjusted over the course of a treatment regimen.
18. The method according to any one of embodiments 1 to 17, wherein the daily dose is adjusted by down-titration, up-titration, interruption or combinations thereof.
19. The method according to any one of embodiments 1 to 18, wherein the daily dose the patient receives is adjusted based on the factors selected from the group consisting of
   a physiological measure selected from the group consisting of LVEF, NYAH, eGFR, change in eGFR, UACR, serum potassium level, sodium level, chloride level, urea level, blood urea nitrogen (BUN), high-sensitivity Troponin T level, natriuretic peptide (NT-proBNP), blood pressure (BP), systolic blood pressure, average heart rate (resting), previous diseases, prior mineralocorticoid receptor antagonist (MRA) use, heart rate (or pulse rate), KCCQ-TSS, body mass index (BMI);
   a comedication selected from the group consisting of loop diuretics, beta-blocker, ACE inhibitor (ACEi), angiotensin-receptor-blocker (ARB), Angiotensin Receptor-Neprilysin Inhibitor (ARNI), calcium channel blockers, Sodium-glucose Cotransporter-2 Inhibitor (SGLT-2i);
   coadministration of a CYP3A4 inducer;
   coadministration of a CYP3A4 inhibitor;
and combinations thereof.
20. The method according to any one of embodiments 1 to 19, wherein the CYP3A4 inhibitor is a strong inhibitor of CYP3A4.
21. The method according to any one of embodiments 1 to 20, wherein the CYP3A4 inhibitor is selected from the group consisting of itraconazole, ketoconazole, ritonavir, nelfinavir, cobicistat, clarithromycin, telithromycin and nefazodone.
22. The method according to any one of embodiments 1 to 19, wherein CYP3A4 inhibitor is a moderate inhibitor of CYP3A4.
23. The method according to any one of embodiments 1 to 19 and 22, wherein CYP3A4 inhibitor is selected from the group consisting of cyclosporin, ciprofloxacin, clotrimazole, diltiazem, dronedarone, erythromycin, fluconazole, and verapamil.
24. The method according to any one of embodiments 1 to 23, wherein the daily dose the patient receives is down-titrated if the potassium level is selected from the group consisting of equal or above 5.5, 5.6, 6, 6.5, 7. 7.5 and 8 mmol/L.
25. The method according to any one of embodiments 1 to 24, wherein the daily dose the patient receives is 40 mg or 30 mg, and wherein the dose is down-titrated to 30 mg, 20 mg or 10 mg.
26. The method according to any one of embodiments 1 to 25, wherein the daily dose the patient receives is up-titrated if the potassium level is equal or below 4.5, 4.8, 4.9, 5 or 5.5 mmol/L.
27. The method according to any one of embodiments 1 to 26, wherein the daily dose the patient receives is 30 mg, 20 mg or 10 mg, and wherein the daily dose is up-titrated to an up-titrated dose of 40 mg, 30 mg or 20 mg.
28. The method according to any one of embodiments 1 to 27, wherein the daily dose the patient receives is down-titrated, if the eGFR decrease is selected from the group consisting of equal to or above 25, 30, 35, 40, 45, 50, and 57%, based on a relative eGFR change from baseline or treatment initiation or prior measurement.
29. The method according to any one of embodiments 1 to 28, wherein the daily dose the patient receives is up-titrated, if the eGFR decrease is selected from equal to or below 5, 10, 15, 25, 30, 35 and 40%, based on the relative eGFR change from baseline or treatment initiation or prior measurement.
30. The method according to any one of embodiments 1 to 29, wherein if the daily dose is 10 or 20 mg, and the potassium level is below 5 mmol/L, then the daily dose is up-titrated to an up-titrated daily dose of 20 mg or 40 mg.
31. The method according to any one of embodiments 1 to 30, wherein if the daily dose is 40 or 20 mg, and the potassium level is equal to or above 5.5 mmol/L, but below 6.0 mmol/L, then the daily dose is down-titrated to a down-titrated daily dose of 20 mg or 10 mg.
32. The method according to any one of embodiments 1 to 31, wherein the daily dosing is interrupted, if the potassium level is equal to or above 6.0 mmol/L.
33. The method according to any one of embodiments 1 to 32, wherein the daily dose is 10 mg and the patient has an eGFR of equal or below 60 mL/min/1.73 m².
34. The method according to any one of embodiments 1 to 33, wherein the patient receives a CYP3A4 inhibitor.
35. The method according to any one of embodiments 1 to 34, wherein the patient receives a CYP3A4 inducer.
36. The method according to any one of embodiments 1 to 35, wherein the daily dose is 5 to 80 mg, and the daily dose is down-titrated to a down-titrated dose of between 5 mg to below 80 mg.
37. The method according to any one of embodiments 1 to 36, wherein the daily dose is between 5 to below 80 mg, and the daily dose is up-titrated to an up-titrated daily dose of between above 5 mg to 80 mg.
38. The method according to any one of embodiments 1 to 37, wherein the patient receives
   a CYP3A4 inducer and
   the compound according to formula (I) in a daily dose selected from the group consisting of 5 mg, 10 mg, 20 mg, 30 mg, 40 mg, and 60 mg wherein the daily dose is adjusted by up-titration to an up-titrated daily dose selected from 10 mg, 20 mg, 30 mg, 40 mg, 60 mg and 80 mg.
      39. The method according to any one of embodiments 1 to 38, wherein the patient receives
   a CYP3A4 inhibitor and
   the compound according to formula (I) in a daily dose selected from the group consisting of 10 mg, 20 mg, 30 mg, 40 mg, 60 mg and 80 mg, wherein the daily dose is adjusted by down-titration to a down-titrated daily dose selected from the group consisting of 2.5 mg, 5 mg, 10 mg, 20 mg, 30 mg, 40 mg, and 60 mg.
40. The method according to any one of embodiments 1 to 39, wherein the patient receives a target daily dose of the compound of Formula (I) of 40 mg, and wherein the target daily dose must be down-titrated to 10 mg per day when co-administered with a strong CYP3A4 inhibitor.
41. The method according to any one of embodiments 1 to 40, wherein the patient receives a target daily dose of the compound of Formula (I) of 40 mg, and wherein the target daily dose must be down-titrated to 20 mg when using with co-administered with a moderate CYP3A4 inhibitor.
42. A method for reducing the risk of cardiovascular death, hospitalization for heart failure, urgent heart failure visits, or combinations thereof in patients with heart failure, comprising administering to the patient a therapeutically effective amount of a compound of Formula (I)

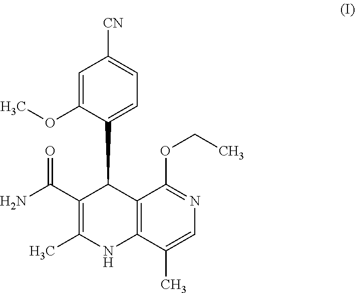 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof, wherein the patient optionally has left ventricular ejection fraction (LVEF) of equal to or above 40%, wherein the method comprises any one of embodiments 2 to 38.
43. A method for improving symptoms or preventing worsening of symptoms of heart failure in patients with heart failure, comprising administering to the patient a therapeutically effective amount of a compound according to Formula (I)

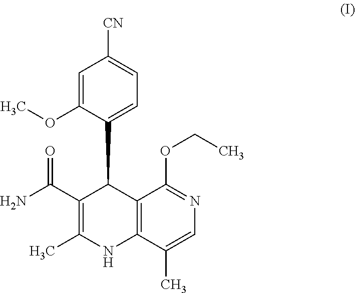 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof, wherein the patient optionally has left ventricular ejection fraction (LVEF) of equal to or above 40%, wherein the method comprises any one of 2 to 38.
44. The method according to any one of embodiments 1 to 43 wherein the compound of Formula (I) is a crystalline form of polymorph I.
45. The method according to any one of embodiments 1 to 44, wherein the compound of Formula (I) is a crystalline form of polymorph I

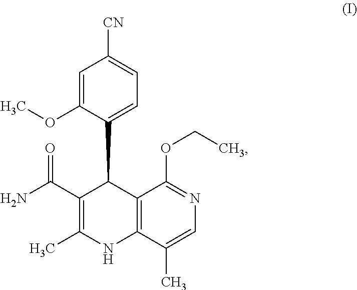 [SMILES: CCOc1ncc(C)c2c1[C@H](c1ccc(C#N)cc1OC)C(C(N)=O)=C(C)N2]

wherein the x-ray diffractogram of the compound exhibits peak maxima of the 2 theta angle at 8.5°, 14.1°, and 19.0°.
46. A method of preventing or treating heart failure in a patient, comprising administering to the patient a therapeutically effective amount of a compound of Formula (I)

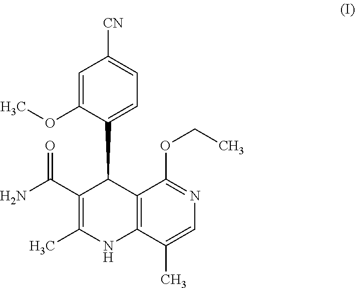 [SMILES: CCOc1ncc(C)c2c1[C@H](c1ccc(C#N)cc1OC)C(C(N)=O)=C(C)N2]

or a hydrate thereof, solvate thereof, pharmaceutically acceptable salt thereof, or a polymorph thereof at a daily dose; and
adjusting the daily dose based on physiological parameters.
47. The method of embodiment 46, wherein the adjusting is selected from the group consisting of reducing the daily dose, increasing the daily dose, and interrupting the daily dose.
48. The method of embodiment 47, wherein the physiological parameters are selected from the group consisting of estimated glomerular filtration rate (eGFR), serum potassium level, and combinations thereof.
49. The method of embodiment 48, wherein the adjusting is reducing the daily dose to a down-titrated daily dose if the serum potassium level is equal to or above 5.5 mmol/L, 5.0 mmol/L, or 4.5 mmol/L; and/or the eGFR is less than or equal to 60 mL/min/1.73 m².
50. The method of embodiment 49, wherein the daily dose is selected from the group consisting of 10 mg, 20 mg, 30 mg, 40 mg, 60 mg and 80 mg; and the down-titrated daily dose is selected from the group consisting of 2.5 mg, 5 mg, 10 mg, 20 mg, 30 mg, 40 mg, and 60 mg.
51. A pharmaceutical dose form for administration to a patient in need thereof, comprising: a compound of Formula (I)

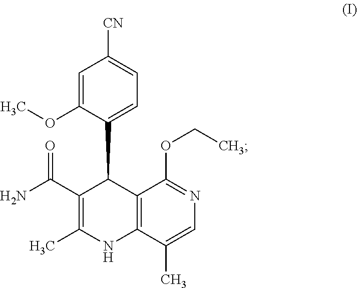 [SMILES: CCOc1ncc(C)c2c1[C@H](c1ccc(C#N)cc1OC)C(C(N)=O)=C(C)N2]

and
a pharmaceutically acceptable excipient.
52. The pharmaceutical dosage form of embodiment 51, wherein the pharmaceutically acceptable excipient is selected from the group consisting of hypromellose, sodium lauryl sulfate, microcrystalline cellulose, lactose monohydrate, croscarmellose, and magnesium stearate.
53. The pharmaceutical dosage form of embodiment 52, wherein the pharmaceutical dosage form is a tablet.
54. The pharmaceutical dosage form of embodiment 53, wherein the compound of Formula (I) is present as a granulate of polymorph A of the compound of Formula (I).
55. The pharmaceutical dosage form of embodiment 54, wherein the compound of Formula (I) is present at a dosage selected from the group consisting of 2.5 mg, 5 mg, 10 mg, 20 mg, 30 mg, 40 mg, 60 mg, and 80 mg.

### ADDITIONAL EMBODIMENTS OF THE DISCLOSURE

1. A method of coadministering finerenone with a strong CYP3A4 inhibitor to a patient in need thereof, comprising administering about 10-60% of a target daily dose of finerenone recommended for an otherwise similar patient who is not co-administered the strong CYP3A4 inhibitor.
2. The method of embodiment 1, comprising administering about 25% of the target daily dose of finerenone recommended for the otherwise similar patient who is not co-administered the strong CYP3A4 inhibitor.
3. The method of embodiment 1, comprising administering about 50% of the target daily dose of finerenone recommended for the otherwise similar patient who is not co-administered the strong CYP3A4 inhibitor.
4. The method of embodiment 1, comprising administering from about 2.5 mg to about 20 mg of finerenone (e.g., about 2.5 mg, about 5 mg, about 10 mg, or about 20 mg).
5. The method of embodiment 1, wherein the target daily dose of finerenone is about 20 mg.
6. The method of embodiment 5, comprising administering from about 2 mg to about 10 mg of finerenone.
7. The method of embodiment 5, comprising administering about 5 mg of finerenone.
8. The method of embodiment 1, wherein the target daily dose of finerenone is about 40 mg.
9. The method of embodiment 8, comprising administering from about 4 mg to about 20 mg of finerenone.
10. The method of embodiment 8, comprising administering 10 mg of finerenone.
11. The method of embodiment 7, wherein the patient in need thereof has chronic kidney disease.
12. The method of embodiment 11, wherein the chronic kidney disease is associated with type 2 diabetes mellitus, associated with type 1 diabetes mellitus, or is non-diabetic chronic kidney disease.
13. The method of any one of embodiments 1-12, wherein the patient in need thereof has heart failure.
14. The method of embodiment 13, wherein the patient with heart failure has an estimated glomerular filtration rate (eGFR) at initiation of finerenone of ≥25 to <60 mL/min/1.73 m², and the patient is administered about 5 mg of finerenone.
15. The method of embodiment 13, wherein the patient with heart failure has an eGFR at initiation of finerenone of ≥60 mL/min/1.73 m², and the method comprises administering about 10 mg of finerenone.
16. The method of any one of embodiments 13-15, wherein the patient in need thereof with heart failure has a left ventricular ejection fraction (LVEF) ≥40%.
17. A method of initiating finerenone treatment in a patient in need thereof that is currently treated with a strong CYP3A4 inhibitor, comprising administering about 10-60% of a starting daily dose of finerenone recommended for an otherwise similar patient who is not co-administered the strong CYP3A4 inhibitor.
18. The method of embodiment 17, wherein the patient has an eGFR at initiation of finerenone of ≥60 mL/min/1.73 m², and the method comprises administering about 2 mg to about 10 mg of finerenone.
19. The method of embodiment 17, wherein the patient has an eGFR at initiation of finerenone of ≥60 mL/min/1.73 m², and the method comprises administering about 5 mg of finerenone.
20. The method of embodiment 17, wherein the patient has an eGFR at initiation of finerenone of ≥25 to <60 mL/min/1.73 m², and the method comprises administering about 1 mg to about 5 mg of finerenone.
21. The method of embodiment 17, wherein the patient has an eGFR at initiation of finerenone of ≥25 to <60 mL/min/1.73 m², and the method comprises administering about 2.5 mg of finerenone.
22. The method of embodiment 17, wherein if the patient has a serum potassium of >4.8-5.5 mEq/L after 4 weeks of treatment, the method comprising administering about 1 mg to about 5 mg of finerenone.
23. The method of embodiment 22, wherein the method comprises administering about 2.5 mg finerenone.
24. The method of embodiment 21, wherein the patient has chronic kidney disease.
25. The method of embodiment 24, wherein the chronic kidney disease is associated with type 2 diabetes mellitus.
26. The method of embodiment 17, if the patient has a serum potassium of ≥5 to <5.5 mEq/L after 4 weeks of treatment and eGFR at initiation of finerenone of ≥25 to <60 mL/min/1.73 m², the method comprises administering about 5 mg of finerenone.
27. The method of embodiment 26, wherein the method comprises administering about 5 mg finerenone.
28. The method of embodiment 27, wherein the patient in need thereof has heart failure.
29. A method of reducing the risk of a comorbidity of heart failure in a patient treated with a strong CYP3A4 inhibitor and in need of treatment with finerenone, comprising administering about 10-60% of a target daily dose of finerenone recommended for an otherwise similar patient who is not co-administered the strong CYP3A4 inhibitor.
30. The method of embodiment 29, wherein the comorbidity comprises hypertension, diabetes mellitus, obesity, chronic kidney disease, coronary artery disease, atrial fibrillation, chronic obstructive pulmonary disease (COPD), anemia, stroke, anxiety, depression, sustained estimated glomerular filtration rate (eGFR) decline, end stage kidney disease, cardiovascular death, non-fatal myocardial infarction, hospitalization, cardiovascular death, urgent heart failure visit, or a combination thereof.
31. The method of embodiment 29, wherein the comorbidity is cardiovascular death, hospitalization or urgent heart failure visit.
32. The method of any one of embodiments 29-31, wherein the patient has LVEF ≥40%.
33. A method of reducing the risk of a comorbidity of chronic kidney disease in a patient treated with a strong CYP3A4 inhibitor and in need of treatment with finerenone, comprising administering about 10-30% of the target daily dose of finerenone.
34. The method of embodiment 33, wherein the comorbidity of chronic kidney disease comprises diabetes mellitus, hypertension, cardiovascular disease, obesity, anemia, a disruption in calcium, phosphorus, sustained estimated glomerular filtration rate (eGFR) decline, end stage kidney disease, cardiovascular death, non-fatal myocardial infarction, hospitalization, vitamin D metabolism, malnutrition, depression, anxiety, dyslipidemia, or a combination thereof.
35. The method of embodiment 33, wherein the comorbidity comprises sustained estimated glomerular filtration rate (eGFR) decline, end stage kidney disease, cardiovascular death, non-fatal myocardial infarction, or hospitalization.
36. Mmethod of any one of embodiments 33-35, wherein the chronic kidney disease is associated with type 2 diabetes.
37. The method of any one of embodiments 29-36, comprising administering about 25% of the target daily dose of finerenone.
38. The method of any one of embodiments 29-37, comprising administering from about 2.5 mg to about 20 mg of finerenone (e.g., about 2.5 mg, about 5 mg, about 10 mg, or about 20 mg). 39. The method of any one of embodiments 29-37, wherein the target daily dose of finerenone is about 20 mg.
40. The method of any one of embodiments 29-39, comprising administering from about 2 mg to about 10 mg of finerenone.
41. The method of any one of embodiments 29-40, comprising administering about 5 mg of finerenone.
42. The method of any one of embodiments 29-37, wherein the target daily dose of finerenone is about 40 mg.
43. The method of any one of embodiments 29-37 and 42, comprising administering from about 4 mg to about 20 mg of finerenone.
44. The method of embodiment 42 or 43, comprising administering 10 mg of finerenone.
45. The method of any one of embodiments 1-44, wherein the patient has IgA nephropathy.
46. The method of any one of embodiments 1-44, wherein the patient in need thereof has chronic kidney disease associated with type 2 diabetes, chronic kidney disease associated with type 1 diabetes, non-diabetic chronic kidney disease, pediatric chronic kidney disease, or a combination thereof.
47. The method of any one of embodiments 1-44, wherein the patient in need thereof has diabetic kidney disease (DKD), anemia, hyperkalemia, frailty, atherosclerotic cardiovascular disease, cardiovascular kidney metabolic syndrome, a cardiovascular disease, chronic kidney disease, a metabolic condition, a worsening heart failure event, or a combination thereof.
48. The method of any one of embodiments 1-47, comprising administering a diuretic, a RAS inhibitor, an SGLT2 inhibitor, a statin, a GLP1 receptor agonist, a beta-blocker, or a calcium channel blocker.
49. The method of any one of embodiments 1-15 and 17-48, wherein the patient in need thereof has an LVEF that is less than 50%.
50. The method of any one of embodiments 1-15 and 17-48, wherein the patient in need thereof has an LVEF that is ≥50 to <60%.
51. The method of any one of embodiments 1-15 and 17-48, wherein the patient in need thereof has an LVEF that is ≥60%.
52. The method of any one of embodiments 1-51, wherein the patient in need thereof is a child from 6 months old to 18 years old.
53. The method of embodiment 52, wherein the child is receiving an angiotensin-converting enzyme (ACE) inhibitor, an angiotensin II receptor blocker (ARB), or a combination thereof.
54. The method of any one of embodiments 52-53, wherein the child has heart failure.
55. The method of any one of embodiments 52-54, wherein the child has left ventricular systolic dysfunction.
56. The method of any one of embodiments 1-51, wherein the patient in need thereof is 18 years old or older.
57. The method of any one of embodiments 1-51 and 56, wherein the patient in need thereof is an adult with primary IgA nephropathy, proteinuria, or a combination thereof. 58. The method of any one of embodiments 1-57, wherein the patient recently had a heart failure event.
59. The method of any one of embodiments 1-58, wherein the patient has obesity.
60. The method of any one of embodiments 56-59, wherein the pateint has left ventricular systolic dysfunction.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the text of the specification, the specification is intended to supersede and/or take precedence over any such contradictory material.

What is claimed is:

1. A method of coadministering finerenone with a strong CYP3A4 inhibitor to a patient in need thereof, comprising administering about 10-60% of a target daily dose of finerenone recommended for an otherwise similar patient who is not co-administered the strong CYP3A4 inhibitor.

2. The method of claim 1, comprising administering about 25% of the target daily dose of finerenone recommended for the otherwise similar patient who is not co-administered the strong CYP3A4 inhibitor.

3. The method of claim 1, comprising administering about 50% of the target daily dose of finerenone recommended for the otherwise similar patient who is not co-administered the strong CYP3A4 inhibitor.

4. The method of claim 1, comprising administering from about 2.5 mg to about 20 mg of finerenone.

5. The method of claim 1, wherein the target daily dose of finerenone is about 20 mg.

6. The method of claim 5, comprising administering from about 2 mg to about 10 mg of finerenone.

7. The method of claim 5, comprising administering about 5 mg of finerenone.

8. The method of claim 1, wherein the target daily dose of finerenone is about 40 mg.

9. The method of claim 8, comprising administering from about 4 mg to about 20 mg of finerenone.

10. The method of claim 8, comprising administering 10 mg of finerenone.

11. The method of claim 7, wherein the patient in need thereof has chronic kidney disease.

12. The method of claim 11, wherein the chronic kidney disease is associated with type 2 diabetes mellitus, associated with type 1 diabetes mellitus, or is non-diabetic chronic kidney disease.

13. The method of claim 1, wherein the patient in need thereof has heart failure.

14. The method of claim 13, wherein the patient with heart failure has an estimated glomerular filtration rate (eGFR) at initiation of finerenone of >25 to <60 mL/min/1.73 m², and the patient is administered about 5 mg of finerenone.

15. The method of claim 13, wherein the patient with heart failure has an eGFR at initiation of finerenone of ≥60 mL/min/1.73 m², and the method comprises administering about 10 mg of finerenone.

16. The method of claim 13, wherein the patient in need thereof with heart failure has a left ventricular ejection fraction (LVEF) ≥40%.

17. A method of initiating finerenone treatment in a patient in need thereof that is currently treated with a strong CYP3A4 inhibitor, comprising administering about 10-60% of a starting daily dose of finerenone recommended for an otherwise similar patient who is not co-administered the strong CYP3A4 inhibitor.

18. The method of claim 17, wherein the patient has an eGFR at initiation of finerenone of ≥60 mL/min/1.73 m², and the method comprises administering about 2 mg to about 10 mg of finerenone.

19. The method of claim 17, wherein the patient has an eGFR at initiation of finerenone of ≥60 mL/min/1.73 m², and the method comprises administering about 5 mg of finerenone.

20. The method of claim 17, wherein the patient has an eGFR at initiation of finerenone of ≥25 to <60 mL/min/1.73 m², and the method comprises administering about 1 mg to about 5 mg of finerenone.

21. The method of claim 17, wherein the patient has an eGFR at initiation of finerenone of ≥25 to <60 mL/min/1.73 m², and the method comprises administering about 2.5 mg of finerenone.

22. The method of claim 17, wherein if the patient has a serum potassium of >4.8-5.5 mEq/L after 4 weeks of treatment, the method comprising administering about 1 mg to about 5 mg of finerenone.

23. The method of claim 22, wherein the method comprises administering about 2.5 mg finerenone.

24. The method of claim 21, wherein the patient has chronic kidney disease.

25. The method of claim 24, wherein the chronic kidney disease is associated with type 2 diabetes mellitus.

26. The method of claim 17, if the patient has a serum potassium of ≥5 to <5.5 mEq/L after 4 weeks of treatment and eGFR at initiation of finerenone of ≥25 to <60 mL/min/1.73 m², the method comprises administering about 5 mg of finerenone.

27. The method of claim 26, wherein the method comprises administering about 5 mg finerenone.

28. The method of claim 27, wherein the patient in need thereof has heart failure.

29. A method of reducing the risk of a comorbidity of heart failure in a patient treated with a strong CYP3A4 inhibitor and in need of treatment with finerenone, comprising administering about 10-60% of a target daily dose of finerenone recommended for an otherwise similar patient who is not co-administered the strong CYP3A4 inhibitor.

30. The method of claim 29, wherein the comorbidity comprises hypertension, diabetes mellitus, obesity, chronic kidney disease, coronary artery disease, atrial fibrillation, chronic obstructive pulmonary disease (COPD), anemia, stroke, anxiety, depression, sustained estimated glomerular filtration rate (eGFR) decline, end stage kidney disease, cardiovascular death, non-fatal myocardial infarction, hospitalization, cardiovascular death, urgent heart failure visit, or a combination thereof.
